(19) United States
(12) Patent Application Publication
(10) Pub. No.: US 2026/0284175 A1
(43) Pub. Date: Sep. 24, 2026

(54) HIV IMMUNOGENIC POLYPEPTIDES AND VACCINES AND USES THEREOF

(71) Applicant: Gilead Sciences, Inc., Foster City, CA (US)

(72) Inventors: Elena Bekerman, Castro Valley, CA (US); Jiani Li, San Francisco, CA (US); Stephen R. Martin, Tiburon, CA (US); Devi SenGupta, San Francisco, CA (US); Evguenia Svarovskaia, Redwood City, CA (US)

(21) Appl. No.: 18/993,457

(22) PCT Filed: Jul. 10, 2023
(86) PCT No.: PCT/US2023/069893

§ 371 Date: Jan. 10, 2025

Related U.S. Application Data
(60) Provisional application No. 63/388,599, filed on Jul. 12, 2022.
(60) Provisional application No. 63/384,020, filed on Nov. 16, 2022.
(60) Provisional application No. 63/485,122, filed on Feb. 15, 2023.

(51) Int. Cl. A61K 39/21; A61K 9/107; A61K 31/7088; A61K 39/00; A61K 39/395; A61K 45/06; A61P 37/04; C07K 14/005; C12N 15/86

(52) U.S. Cl. CPC A61K 39/21; A61K 9/1075; A61K 31/7088; A61K 39/3955; A61K 45/06; A61P 37/04; C07K 14/005; C12N 15/86; A61K 2039/5256; A61K 2039/545; A61K 2039/55522; A61K 2039/55527; A61K 2039/55533; A61K 2039/55538; C12N 2710/10343; C12N 2740/16022; C12N 2740/16034

(57) ABSTRACT

The present disclosure relates HIV immunogenic polypeptides, polynucleotides encoding such HIV immunogenic polypeptides, self-amplifying messenger RNA (SAM) vectors comprising such polynucleotides, and viral vectors comprising such polynucleotides.

### CROSS-REFERENCE TO RELATED APPLICATIONS

This application claims the benefit under 35 U.S.C. § 119 (e) of U.S. Provisional Application No. 63/388,599, filed on Jul. 12, 2022, U.S. Provisional Application No. 63/384,020, filed on Nov. 16, 2022, and U.S. Provisional Application No. 63/485,122, filed on Feb. 15, 2023, each of which is incorporated herein in its entirety for all purposes.

### SEQUENCE LISTING

The instant application contains a Sequence Listing which has been submitted electronically in .XML file format and is hereby incorporated by reference in its entirety. Said. XML copy, created on Jun. 27, 2023, is named 1444-WO-PCT.xml and is 691,866 bytes in size.

### FIELD

The present disclosure relates to novel HIV immunogens, polynucleotides encoding such HIV immunogens, and HIV vaccines comprising adenoviral vectors and/or self-amplifying messenger RNA (SAM) vectors. These HIV vaccines may comprise a chimpanzee adenoviral (ChAd) vector and/or a SAM vector.

### BACKGROUND

Human immunodeficiency virus type 1 (HIV-1) infection contributes to global morbidity and mortality with 38.0 million people globally and 1.1 million people in the United States (US) living with HIV (Joint United Nations Programme on HIV/AIDS (UNAIDS). UNAIDS Data. Geneva, Switzerland. 2020). Antiretroviral therapy (ART) is highly effective at suppressing viral replication in people with HIV (PWH). However, cessation of ART leads to rapid rebound of viral replication; therefore, PWH must remain on ART throughout their lifetimes. The rebound in viremia stems from a persistent reservoir of HIV-infected cells that harbor full-length, replication-competent proviral HIV DNA that is not eliminated by ART. The HIV reservoir is established very early in HIV infection, and a significant portion of the reservoir is transcriptionally silent (i.e., latent) at any given time. As such, the latent HIV reservoir cannot be recognized and eliminated by the immune system. Latent HIV proviruses can be found in all CD4+ T-cell subsets (including long-lived memory cells), monocytes, and macrophages in peripheral blood and tissues. Viral rebound most likely results from stochastic activation of latently infected cells in the absence of viral suppression (Sengupta and Siliciano, Targeting the Latent Reservoir for HIV-1. Immunity 2018; 48 (5): 872-95). Thus, there is a need for the development of novel therapies that can target and destroy cells latently infected with HIV, the main barrier to HIV cure, which could reduce and ultimately eliminate the HIV reservoir and lead to long-term viral remission.

### SUMMARY

Disclosed herein are polynucleotides comprising any one of the HIV immunogen nucleic acid sequences of SEQ ID NOs: 524-526.

Disclosed herein are polynucleotides comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the HIV immunogen nucleic acid sequences of SEQ ID NOs: 524-526, wherein percent identity is across the full length of the HIV immunogen nucleic acid sequence of SEQ ID NOs: 524-526.

Disclosed herein are polynucleotides comprising an HIV immunogen nucleic acid sequence that encodes any one of the HIV immunogen amino acid sequences of SEQ ID NOS: 527-528.

Disclosed herein are polynucleotides comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 527-528, wherein percent identity is across the full length of the nucleic acid sequence of the polypeptide encoding any one of the HIV immunogen amino acid sequence of SEQ ID NOs: 527-528.

In some embodiments, the polynucleotide comprises cDNA or mRNA.

Disclosed herein are self-amplifying RNA (SAM or saRNA) comprising any of the polynucleotides disclosed herein.

In some embodiments, the SAM comprises a polynucleotide comprising any one of the HIV immunogen nucleic acid sequences of SEQ ID NOs: 524-526.

In some embodiments, the SAM comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the HIV immunogen nucleic acid sequences of SEQ ID NOs: 524-526, wherein percent identity is across the full length of the HIV immunogen nucleic acid sequence of SEQ ID NOs: 524-526.

In some embodiments, the SAM comprises a polynucleotide comprising an HIV immunogen nucleic acid sequence that encodes any one of the HIV immunogen amino acid sequences of SEQ ID NOs: 527-528.

In some embodiments, the SAM comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 527-528, wherein percent identity is across the full length of the nucleic acid sequence of the polypeptide encoding any one of the HIV immunogen amino acid sequence of SEQ ID NOs: 527-528.

In some embodiments, the SAM is derived from a virus.

In some embodiments, the virus is an RNA virus.

In some embodiments, the RNA virus is positive-sense single-stranded RNA virus.

In some embodiments, the virus is selected from an alphavirus, flavivirus, nidovirus, nodamura virus, and picornavirus.

In some embodiments, the alphavirus is selected from an Old World (OW) alphavirus and New World (NW) alpha virus.

In some embodiments, the OW alphavirus is selected from Chikunguyna virus (CHIKV), Ross River virus (RRV), Semliki Forest virus (SFV), and Sindbis virus (SINV).

In some embodiments, the NW alphavirus is selected from Venezuelan equine encephalitis virus (VEEV), eastern equine encephalitis virus (EEEV), and western equine encephalitis virus (WEEV).

In some embodiments, the SAM is derived from Venezuelan equine encephalitis virus (VEEV).

In some embodiments, the virus comprises a modified viral genome.

In some embodiments, the modified viral genome comprises a deletion of one or more genes encoding one or more viral structural proteins.

In some embodiments, the modified viral genome is produced by replacing one or more viral structural proteins with any of the polynucleotides disclosed herein.

In some embodiments, the modified viral genome is produced by replacing one or more viral structural proteins with a polynucleotide comprising the nucleic acid sequence of SEQ ID NOs: 524-526.

In some embodiments, the modified viral genome is produced by replacing one or more viral structural proteins with a polynucleotide comprising a codon-optimized nucleic acid sequence of the nucleic acid sequence of SEQ ID NOs: 524-526.

In some embodiments, the modified viral genome is produced by replacing one or more viral structural proteins with a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 524-526, wherein percent identity is across the full length of the polypeptide of SEQ ID NOs: 524-526.

In some embodiments, the modified viral genome is produced by replacing one or more viral structural proteins with a polynucleotide comprising a codon-optimized nucleic acid sequence that encodes any of the polypeptides of SEQ ID NOs: 345-371, 373-377, 407-411, 422-423, 430-435, 527, and 528.

In some embodiments, the modified viral genome is produced by replacing one or more viral structural proteins with a polynucleotide that encodes any of the polypeptides of SEQ ID NOs: 345-371, 373-377, 407-411, 422-423, 430-435, 527, and 528.

In some embodiments, the modified viral genome is produced by replacing one or more viral structural proteins with a polynucleotide comprising a codon-optimized nucleic acid sequence that encodes any of the polypeptides of SEQ ID NOs: 345-371, 373-377, 407-411, 422-423, 430-435, 527, and 528.

In some embodiments, the modified viral genome is produced by replacing one or more viral structural proteins with a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 345-371, 373-377, 407-411, 422-423, 430-435, 527, and 528, wherein percent identity is across the full length of the polypeptide of SEQ ID NOs: 345-371, 373-377, 407-411, 422-423, 430-435, 527, and 528.

In some embodiments, the modified viral genome is produced by replacing one or more viral structural proteins with a polynucleotide encoding any of the polypeptides of SEQ ID NOS: 1-344.

In some embodiments, the modified viral genome is produced by replacing one or more viral structural proteins with a polynucleotide comprising a codon-optimized nucleic acid sequence that encodes any one of the polypeptides of SEQ ID NOs: 1-344.

In some embodiments, the modified viral genome is produced by replacing one or more viral structural proteins with a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 1-344, wherein percent identity is across the full length of the polypeptide of 1-344.

In some embodiments, the virus is replication deficient.

Further disclosed herein are self-amplifying RNAs (SAMs or saRNAs) comprising a polynucleotide, wherein the polynucleotide comprises any one of the nucleic acid sequences of SEQ ID NOs: 522-523.

Further disclosed herein are self-amplifying RNAs (SAMs or saRNAs) comprising a polynucleotide, wherein the polynucleotide comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523.

Further disclosed herein are self-amplifying RNAs (SAMs or saRNAs) comprising a polynucleotide, wherein the polynucleotide comprises any one of the nucleic acid sequences of SEQ ID NOs: 524-526.

Further disclosed herein are self-amplifying RNAs (SAMs or saRNAs) comprising a polynucleotide, wherein the polynucleotide comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526.

Further disclosed herein are self-amplifying RNAs (SAMs or saRNAs) comprising a polynucleotide, wherein the polynucleotide encodes any one of the amino acid sequences of SEQ ID NOs: 527-528.

Further disclosed herein are self-amplifying RNAs (SAMs or saRNAs) comprising a polynucleotide that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid sequences of SEQ ID NOs: 527-528, wherein percent identity is across the full length of the amino acid sequence of SEQ ID NOs: 527-528.

Further disclosed herein are self-amplifying RNAs (SAMs or saRNAs) comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (e) a fourth polynucleotide sequence comprising nucleotides 10667 to 10952 of SEQ ID NO: 522; and (f) a polyA region.

Further disclosed herein are self-amplifying RNAs (SAMs or saRNAs) comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 10667 to 10952 of SEQ ID NO: 522; and (f) a polyA region.

In some embodiments, the third polynucleotide sequence encodes two or more polypeptide segments.

In some embodiments, the third polynucleotide sequence encodes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344.

In some embodiments, one or more of the polypeptide segments is abutted or fused to an adjacent polypeptide segment.

In some embodiments, one or more of the polypeptide segments is joined to an adjacent polypeptide segment by one or more peptide linkers.

In some embodiments, the one or more peptide linkers is selected from one or more of a polyalanine linker, a polyglycine linker, a cleavable linker, a flexible linker, a rigid linker, a Nef linking sequence, and combinations thereof.

In some embodiments, the polyalanine linker comprises or consists of 2 or 3 contiguous alanine residues, e.g. AA, AAA, AAY or AAX, wherein X is any amino acid (e.g. A, C, D, E, F, G, H, I, K, L, M, N, P, Q, R, S, T, V, Y).

In some embodiments, the flexible linker or polyglycine linker comprises or consists of GG, GGS, GSG or GGGS (SEQ ID NO: 421).

In some embodiments, the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof.

In some embodiments, the cleavable linker comprises or consists of a furin recognition/cleavage site selected from the group consisting of RAKR (SEQ ID NO: 381), REKR (SEQ ID NO: 382) and RRKR (SEQ ID NO: 383).

In some embodiments, the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388).

In some embodiments, the cleavable linker comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388).

In some embodiments, the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), wherein percent identity is across the full length of SEQ ID NOs: 389-391.

In some embodiments, the Nef linking sequence comprises or consists of an amino acid sequence selected from VHAGPIA (SEQ ID NO: 389), VHAGPVA (SEQ ID NO: 390) and GALDI (SEQ ID NO: 391).

In some embodiments, the promoter sequence comprises a polynucleotide sequence of SEQ ID NO: 529.

Further disclosed herein are expression cassettes comprising any of the polypeptides or SAMs disclosed herein, operably linked to one or more regulatory sequences.

In some embodiments, the expression cassette comprises a polynucleotide comprising the nucleic acid sequence of SEQ ID NOs: 524-526, operably linked to one or more regulatory sequences.

In some embodiments, the expression cassette comprises a polynucleotide comprising a codon-optimized nucleic acid sequence of the nucleic acid sequence of SEQ ID NOs: 524-526, operably linked to one or more regulatory sequences.

In some embodiments, the expression cassette comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 524-526, wherein percent identity is across the full length of the polypeptide of SEQ ID NOs: 524-526, and wherein the polynucleotide is operably linked to one or more regulatory sequences.

In some embodiments, the expression cassette comprises a polynucleotide comprising a codon-optimized nucleic acid sequence that encodes any of the polypeptides of SEQ ID NOs: 345-371, 373-377, 407-411, 422-423, 430-435, 527, and 528, operably linked to one or more regulatory sequences.

In some embodiments, the expression cassette comprises a polynucleotide that encodes any of the polypeptides of SEQ ID NOs: 345-371, 373-377, 407-411, 422-423, 430-435, 527, and 528, operably linked to one or more regulatory sequences.

In some embodiments, the expression cassette comprises a polynucleotide comprising a codon-optimized nucleic acid sequence that encodes any of the polypeptides of SEQ ID NOs: 345-371, 373-377, 407-411, 422-423, 430-435, 527, and 528, operably linked to one or more regulatory sequences.

In some embodiments, the expression cassette comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 345-371, 373-377, 407-411, 422-423, 430-435, 527, and 528, wherein percent identity is across the full length of the polypeptide of SEQ ID NOs: 345-371, 373-377, 407-411, 422-423, 430-435, 527, and 528, wherein the polynucleotide is operably linked to one or more regulatory sequences.

In some embodiments, the expression cassette comprises a polynucleotide encoding any of the polypeptides of SEQ ID NOs: 1-344, operably linked to one or more regulatory sequences.

In some embodiments, the expression cassette comprises a polynucleotide comprising a codon-optimized nucleic acid sequence that encodes any one of the polypeptides of SEQ ID NOS: 1-344, operably linked to one or more regulatory sequences.

In some embodiments, the expression cassette comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 1-344, wherein percent identity is across the full length of the polypeptide of 1-344, wherein the polynucleotide is operably linked to one or more regulatory sequences.

In some embodiments, the polynucleotide is operably linked to and under the control of a constitutive promoter.

In some embodiments, the promoter is selected from a CMV promoter, a CAG promoter, an EF1a promoter, and a 26S sub-genomic promoter.

In some embodiments, the promoter comprises any of the promoter sequences of SEQ ID NOs: 529-530.

Further disclosed herein are lipid nanoparticles (LNPs) comprising any of the polynucleotides, SAMs, expression cassettes, or expression vectors disclosed herein.

In some embodiments, the LNP comprises a polynucleotide comprising any one of the nucleic acid sequences of SEQ ID NOs: 522-523, or a SAM, expression cassette, or expression vector comprising such polynucleotide.

In some embodiments, the LNP comprises a polynucleotide comprising a codon-optimized nucleic acid sequence of any one of the nucleic acid sequences of SEQ ID NOs: 522-523, or a SAM, expression cassette, or expression vector comprising such polynucleotide.

In some embodiments, the LNP comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523, or a SAM, expression cassette, or expression vector comprising such polynucleotide.

In some embodiments, the LNP comprises a polynucleotide comprising any one of the nucleic acid sequences of SEQ ID NOs: 524-526, or a SAM, expression cassette, or expression vector comprising such polynucleotide.

In some embodiments, the LNP comprises a polynucleotide comprising a codon-optimized nucleic acid sequence of any one of the nucleic acid sequences of SEQ ID NOs: 524-526, or a SAM, expression cassette, or expression vector comprising such polynucleotide.

In some embodiments, the LNP comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526, or a SAM, expression cassette, or expression vector comprising such polynucleotide.

In some embodiments, the LNP comprises a polynucleotide comprising a nucleic acid sequence that encodes any one of the polypeptides of SEQ ID NOs: 527-528, or a SAM, expression cassette, or expression vector comprising such polynucleotide.

In some embodiments, the LNP comprises a polynucleotide comprising a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid sequences of SEQ ID NOs: 527-528, wherein percent identity is across the full length of the amino acid sequence of SEQ ID NOs: 527-528, or a SAM, expression cassette, or expression vector comprising such polynucleotide.

In some embodiments, the LNP comprises a SAM comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (e) a fourth polynucleotide sequence comprising nucleotides 10667 to 10952 of SEQ ID NO: 522; and (f) a polyA region.

In some embodiments, the LNP comprises a SAM comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 10667 to 10952 of SEQ ID NO: 522; and (f) a polyA region.

In some embodiments, the LNP comprises or is synthesized from an ionizable lipid, phospholipid, cholesterol, PEGylated lipid, or any combination thereof.

In some embodiments, the LNP comprises or is synthesized from cationic lipid 1,2-dioleoyl-3-timethylammonium-propane (DOTAP).

Further disclosed herein are polymeric nanoparticles (PNPs) comprising any of the polynucleotides, SAMs, expression cassettes, or expression vectors disclosed herein.

In some embodiments, the PNP comprises a polynucleotide comprising any one of the nucleic acid sequences of SEQ ID NOs: 522-523, or a SAM, expression cassette, or expression vector comprising such polynucleotide.

In some embodiments, the PNP comprises a polynucleotide comprising a codon-optimized nucleic acid sequence of any one of the nucleic acid sequences of SEQ ID NOs: 522-523, or a SAM, expression cassette, or expression vector comprising such polynucleotide.

In some embodiments, the PNP comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523, or a SAM, expression cassette, or expression vector comprising such polynucleotide.

In some embodiments, the PNP comprises a polynucleotide comprising any one of the nucleic acid sequences of SEQ ID NOs: 524-526, or a SAM, expression cassette, or expression vector comprising such polynucleotide.

In some embodiments, the PNP comprises a polynucleotide comprising a codon-optimized nucleic acid sequence of any one of the nucleic acid sequences of SEQ ID NOs: 524-526, or a SAM, expression cassette, or expression vector comprising such polynucleotide.

In some embodiments, the PNP comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526, or a SAM, expression cassette, or expression vector comprising such polynucleotide.

In some embodiments, the PNP comprises a polynucleotide comprising a nucleic acid sequence that encodes any one of the polypeptides of SEQ ID NOs: 527-528, or a SAM, expression cassette, or expression vector comprising such polynucleotide.

In some embodiments, the PNP comprises a polynucleotide comprising a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid sequences of SEQ ID NOs: 527-528, wherein percent identity is across the full length of the amino acid sequence of SEQ ID NOs: 527-528, or a SAM, expression cassette, or expression vector comprising such polynucleotide.

In some embodiments, the PNP comprises a SAM comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (e) a fourth polynucleotide sequence comprising nucleotides 10667 to 10952 of SEQ ID NO: 522; and (f) a polyA region.

In some embodiments, the PNP comprises a SAM comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 10667 to 10952 of SEQ ID NO: 522; and (f) a polyA region.

In some embodiments, the PNP comprises or is synthesized from non-degradable polymers, degradable polymers, natural materials, synthetic materials, monomers, dendrimers, or any combination thereof.

In some embodiments, the PNP comprises or is synthesized from poly(ethylene glycol) (PEG), poly(dimethylsiloxane) (PDMS), polyethyleneimine (PEI), poly(amidoamine) (PAMAM), poly(dimethylaminoethyl) acrylate (pDMAEA), orthenine-derived dendrimers, or any combination thereof.

In some embodiments, the PNP comprises or is synthesized from mannosylated-PEI polyplexes.

In some embodiments, the PNP comprises or is synthesized from a bioreducible, linear, cationic polymer (pABOL).

Further disclosed herein are nanoemulsions comprising any of the polynucleotides, SAMs, expression cassettes, or expression vectors disclosed herein.

In some embodiments, the nanoemulsion comprises a polynucleotide comprising any one of the nucleic acid sequences of SEQ ID NOs: 522-523, or a SAM, expression cassette, or expression vector comprising such polynucleotide.

In some embodiments, the nanoemulsion comprises a polynucleotide comprising a codon-optimized nucleic acid sequence of any one of the nucleic acid sequences of SEQ ID NOs: 522-523, or a SAM, expression cassette, or expression vector comprising such polynucleotide.

In some embodiments, the nanoemulsion comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523, or a SAM, expression cassette, or expression vector comprising such polynucleotide.

In some embodiments, the nanoemulsion comprises a polynucleotide comprising any one of the nucleic acid sequences of SEQ ID NOs: 524-526, or a SAM, expression cassette, or expression vector comprising such polynucleotide.

In some embodiments, the nanoemulsion comprises a polynucleotide comprising a codon-optimized nucleic acid sequence of any one of the nucleic acid sequences of SEQ ID NOs: 524-526, or a SAM, expression cassette, or expression vector comprising such polynucleotide.

In some embodiments, the nanoemulsion comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526, or a SAM, expression cassette, or expression vector comprising such polynucleotide.

In some embodiments, the nanoemulsion comprises a polynucleotide comprising a nucleic acid sequence that encodes any one of the polypeptides of SEQ ID NOs: 527-528, or a SAM, expression cassette, or expression vector comprising such polynucleotide.

In some embodiments, the nanoemulsion comprises a polynucleotide comprising a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid sequences of SEQ ID NOs: 527-528, wherein percent identity is across the full length of the amino acid sequence of SEQ ID NOs: 527-528, or a SAM, expression cassette, or expression vector comprising such polynucleotide.

In some embodiments, the nanoemulsion comprises a SAM comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOS: 345-371, 373-377, 407-411, 422-423, 430-435, 527, and 528; (e) a fourth polynucleotide sequence comprising nucleotides 10667 to 10952 of SEQ ID NO: 522; and (f) a polyA region.

In some embodiments, the nanoemulsion comprises a SAM comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 10667 to 10952 of SEQ ID NO: 522; and (f) a polyA region.

In some embodiments, the nanoemulsion is a water-in-oil emulsion.

In some embodiments, the nanoemulsion comprises squalene, sorbitan trioleate, polysorbate 80, DOTAP, or any combination thereof.

Further disclosed herein are expression vectors comprising any of the polynucleotides, SAMs, or expression cassettes disclosed herein.

In some embodiments, the expression vector comprises a polynucleotide comprising any one of the nucleic acid sequences of SEQ ID NOs: 522-523, or a SAM or expression cassette comprising such polynucleotide.

In some embodiments, the expression vector comprises a polynucleotide comprising a codon-optimized nucleic acid sequence of any one of the nucleic acid sequences of SEQ ID NOS: 522-523, or a SAM or expression cassette comprising such polynucleotide.

In some embodiments, the expression vector comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523, or a SAM or expression cassette comprising such polynucleotide.

In some embodiments, the expression vector comprises a polynucleotide comprising any one of the nucleic acid sequences of SEQ ID NOs: 524-526, or a SAM or expression cassette comprising such polynucleotide.

In some embodiments, the expression vector comprises a polynucleotide comprising a codon-optimized nucleic acid sequence of any one of the nucleic acid sequences of SEQ ID NOS: 524-526, or a SAM or expression cassette comprising such polynucleotide.

In some embodiments, the expression vector comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526, or a SAM or expression cassette comprising such polynucleotide.

In some embodiments, the expression vector comprises a polynucleotide comprising a nucleic acid sequence that encodes any one of the polypeptides of SEQ ID NOs: 527-528, or a SAM or expression cassette comprising such polynucleotide.

In some embodiments, the expression vector comprises a polynucleotide comprising a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid sequences of SEQ ID NOs: 527-528, wherein percent identity is across the full length of the amino acid sequence of SEQ ID NOs: 527-528, or a SAM or expression cassette comprising such polynucleotide.

In some embodiments, the expression vector comprises a SAM comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOS: 345-371, 373-377, 407-411, 422-423, 430-435, 527, and 528; (e) a fourth polynucleotide sequence comprising nucleotides 10667 to 10952 of SEQ ID NO: 522; and (f) a polyA region.

In some embodiments, the expression vector comprises a SAM comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 10667 to 10952 of SEQ ID NO: 522; and (f) a polyA region.

In some embodiments, the expression vector is a plasmid vector, a bacterial vector or a viral expression vector.

In some embodiments, the expression vector is a viral expression vector.

In some embodiments, the viral expression vector is a DNA virus or an RNA virus.

In some embodiments, the viral expression vector is replication defective, replication deficient, replication attenuated or replication competent.

In some embodiments, the viral expression vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus.

In some embodiments, the viral expression vector is from a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus).

In some embodiments, the viral expression vector is an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV).

In some embodiments, the viral expression vector is an arenavirus vector selected from Lymphocytic choriomeningitis mammarenavirus (LCMV) or Cali mammarenavirus (a.k.a. Pichinde mammarenavirus or Pichinde arenavirus).

In some embodiments, the arenavirus vector comprises a bi-segmented genome.

In some embodiments, the arenavirus vector comprises a tri-segmented genome.

In some embodiments, the viral expression vector is a human adenovirus or a simian adenovirus.

In some embodiments, the simian adenovirus is selected from a chimpanzee adenovirus, a gorilla adenovirus, and a rhesus adenovirus.

In some embodiments, the viral expression vector is an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (ChAd), gorilla adenovirus, and rhesus adenovirus.

In some embodiments, the ChAd is selected from ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), and ChAd147 (AdC147).

In some embodiments, the ChAd is ChAd68.

In some embodiments, the gorilla adenovirus is selected from GC44, GC45, and GC46.

In some embodiments, the rhesus adenovirus (RhAd) is selected from RhAd51, RhAd52, RhAd53, RhAd54, RhAd55, RhAd56, RhAd57, RhAd58, RhAd59, RhAd60, RhAd61, RhAd62, RhAd63, RhAd64, RhAd65 and RhAd66.

In some embodiments, the human adenovirus or simian adenovirus comprises a modification of one or more adenoviral genes.

In some embodiments, the one or more adenoviral genes are selected from E1, E3, and E4ORF2-4.

In some embodiments, the modification comprises a deletion, substitution, or insertion of one or more nucleotides into the one or more adenoviral genes.

In some embodiments, the human adenovirus or simian adenovirus comprises a deletion of at least a portion of the E1 region.

In some embodiments, the polynucleotide is inserted into the deletion of at least a portion of the E1 region.

In some embodiments, the expression vector comprises any one of the nucleic acid sequences of SEQ ID NOs: 520-521.

In some embodiments, the expression vector comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521.

Further disclosed herein is an expression vector comprising any one of the nucleic acid sequences of SEQ ID NOs: 524-526.

Further disclosed herein is an expression vector comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOS: 524-526.

Further disclosed herein is an expression vector comprising a polynucleotide sequence that encodes for any one of the amino acid sequences of SEQ ID NOs: 527-528.

Further disclosed herein is an expression vector comprising a polynucleotide sequence that encodes for polynucleotide that is at least 80%, 85%, 86%, 87%, 88%, 89%, 90%, 91%, 92%, 93%, 94%, 95%, 96%, 97%, 98%, 99%, or 100% identical to any one of the amino acid sequences of SEQ ID NOs: 527-528, wherein percent identity is across the full length of the amino acid sequence of SEQ ID NOs: 527-528.

Further disclosed herein is an expression vector comprising: (a) a first polynucleotide sequence comprising nucleotides 1 to 593 of SEQ ID NO: 520; (b) a promoter sequence; (b) a second polynucleotide sequence comprising nucleotides 1196 to 1255 of SEQ ID NO: 520; (c) a third polynucleotide sequence encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (d) a fourth polynucleotide sequence comprising nucleotides 4352 to 4395 of SEQ ID NO: 520; (e) a polyA sequence; and (f) a fifth polynucleotide sequence comprising nucleotides 4620 to 32350 of SEQ ID NO: 520.

Further disclosed herein is an expression vector comprising: (a) a first polynucleotide sequence comprising nucleotides 1 to 593 of SEQ ID NO: 520; (b) a promoter sequence; (c) a second polynucleotide sequence comprising nucleotides 1196 to 1255 of SEQ ID NO: 520; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 4352 to 4395 of SEQ ID NO: 520; (f) a polyA sequence; and (g) a fifth polynucleotide sequence comprising nucleotides 4620 to 32350 of SEQ ID NO: 520.

In some embodiments, the third polynucleotide sequence encodes two or more polypeptide segments.

In some embodiments, the third polynucleotide sequence encodes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344.

In some embodiments, one or more of the polypeptide segments is abutted or fused to an adjacent polypeptide segment.

In some embodiments, one or more of the polypeptide segments is joined to an adjacent polypeptide segment by one or more peptide linkers.

In some embodiments, the one or more peptide linkers is selected from one or more of a polyalanine linker, a polyglycine linker, a cleavable linker, a flexible linker, a rigid linker, a Nef linking sequence, and combinations thereof.

In some embodiments, the polyalanine linker comprises or consists of 2 or 3 contiguous alanine residues, e.g. AA, AAA, AAY or AAX, wherein X is any amino acid (e.g. A, C, D, E, F, G, H, I, K, L, M, N, P, Q, R, S, T, V, Y).

In some embodiments, the flexible linker or polyglycine linker comprises or consists of GG, GGS, GSG or GGGS (SEQ ID NO: 421).

In some embodiments, the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof.

In some embodiments, the cleavable linker comprises or consists of a furin recognition/cleavage site selected from the group consisting of RAKR (SEQ ID NO: 381), REKR (SEQ ID NO: 382) and RRKR (SEQ ID NO: 383).

In some embodiments, the cleavable linker comprises or consists of the amino acid sequence that is at least 90%, 91%, 92%, 93%, 94%, 95%, 96%, 97%, 98% or at least 99% identical to ATNFSLLKQAGDVEENPGP ID NO: (SEQ 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388), wherein percent identity is across the full length of SEQ ID NOs: 384-388.

In some embodiments, the cleavable linker comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388).

In some embodiments, the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), wherein percent identity is across the full length of SEQ ID NOs: 389-391.

In some embodiments, the Nef linking sequence comprises or consists of an amino acid sequence selected from VHAGPIA (SEQ ID NO: 389), VHAGPVA (SEQ ID NO: 390) and GALDI (SEQ ID NO: 391).

In some embodiments, the promoter sequence comprises a polynucleotide sequence of SEQ ID NO: 529.

Further disclosed herein are viral vectors comprising any of the polynucleotides, SAMs, expression cassettes, or expression vectors disclosed herein.

In some embodiments, the viral vector comprises a polynucleotide comprising any one of the nucleic acid sequences of SEQ ID NOs: 522-523, or a SAM, expression cassette, or expression vector comprising such polynucleotide.

In some embodiments, the viral vector comprises a polynucleotide comprising a codon-optimized nucleic acid sequence of any one of the nucleic acid sequences of SEQ ID NOs: 522-523, or a SAM, expression cassette, or expression vector comprising such polynucleotide.

In some embodiments, the viral vector comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523, or a SAM, expression cassette, or expression vector comprising such polynucleotide.

In some embodiments, the viral vector comprises a polynucleotide comprising any one of the nucleic acid sequences of SEQ ID NOs: 524-526, or a SAM, expression cassette, or expression vector comprising such polynucleotide.

In some embodiments, the viral vector comprises a polynucleotide comprising a codon-optimized nucleic acid sequence of any one of the nucleic acid sequences of SEQ ID NOs: 524-526, or a SAM, expression cassette, or expression vector comprising such polynucleotide.

In some embodiments, the viral vector comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526, or a SAM, expression cassette, or expression vector comprising such polynucleotide.

In some embodiments, the viral vector comprises a polynucleotide comprising a nucleic acid sequence that encodes any one of the polypeptides of SEQ ID NOs: 527-528, or a SAM, expression cassette, or expression vector comprising such polynucleotide.

In some embodiments, the viral vector comprises a polynucleotide comprising a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid sequences of SEQ ID NOs: 527-528, wherein percent identity is across the full length of the amino acid sequence of SEQ ID NOs: 527-528, or a SAM, expression cassette, or expression vector comprising such polynucleotide.

In some embodiments, the viral vector comprises a SAM comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (e) a fourth polynucleotide sequence comprising nucleotides 10667 to 10952 of SEQ ID NO: 522; and (f) a polyA region.

In some embodiments, the viral vector comprises a SAM comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 10667 to 10952 of SEQ ID NO: 522; and (f) a polyA region.

In some embodiments, the viral vector is derived from or based on a DNA virus or an RNA virus.

In some embodiments, the viral vector is replication defective, replication deficient, replication attenuated or replication competent.

In some embodiments, the viral expression vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus.

In some embodiments, the viral vector is derived from or based on a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus).

In some embodiments, the viral vector is derived from or based on an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV).

In some embodiments, the viral vector is derived from or based on an arenavirus vector selected from Lymphocytic choriomeningitis mammarenavirus (LCMV) or Cali mammarenavirus (a.k.a. Pichinde mammarenavirus or Pichinde arenavirus).

In some embodiments, the arenavirus vector comprises a bi-segmented genome.

In some embodiments, the arenavirus vector comprises a tri-segmented genome.

In some embodiments, the viral vector is a human adenovirus or a simian adenovirus.

In some embodiments, the simian adenovirus is selected from a chimpanzee adenovirus, a gorilla adenovirus, and a rhesus adenovirus.

In some embodiments, the viral vector is derived from or based on an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (ChAd), gorilla adenovirus, and rhesus adenovirus.

In some embodiments, the ChAd is selected from ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), and ChAd147 (AdC147).

In some embodiments, the ChAd is ChAd68.

In some embodiments, the gorilla adenovirus is selected from GC44, GC45, and GC46.

In some embodiments, the rhesus adenovirus (RhAd) is selected from RhAd51, RhAd52, RhAd53, RhAd54, RhAd55, RhAd56, RhAd57, RhAd58, RhAd59, RhAd60, RhAd61, RhAd62, RhAd63, RhAd64, RhAd65 and RhAd66.

In some embodiments, the human adenovirus or simian adenovirus comprises a modification of one or more adenoviral genes.

In some embodiments, the one or more adenoviral genes are selected from E1, E3, and E4ORF2-4.

In some embodiments, the modification comprises a deletion, substitution, or insertion of one or more nucleotides into the one or more adenoviral genes.

In some embodiments, the human adenovirus or simian adenovirus comprises a deletion of at least a portion of the E1 region.

In some embodiments, the polynucleotide is inserted into the deletion of at least a portion of the E1 region.

In some embodiments, the viral vector comprises any one of the nucleic acid sequences of SEQ ID NOs: 520-521.

In some embodiments, the viral vector comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521.

In some embodiments, the viral vector comprises an expression vector comprising: (a) a first polynucleotide sequence comprising nucleotides 1 to 593 of SEQ ID NO: 520; (b) a promoter sequence; (b) a second polynucleotide sequence comprising nucleotides 1196 to 1255 of SEQ ID NO: 520; (c) a third polynucleotide sequence encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (d) a fourth polynucleotide sequence comprising nucleotides 4352 to 4395 of SEQ ID NO: 520; (e) a polyA sequence; and (f) a fifth polynucleotide sequence comprising nucleotides 4620 to 32350 of SEQ ID NO: 520.

In some embodiments, the viral vector comprises an expression vector comprising: (a) a first polynucleotide sequence comprising nucleotides 1 to 593 of SEQ ID NO: 520; (b) a promoter sequence; (c) a second polynucleotide sequence comprising nucleotides 1196 to 1255 of SEQ ID NO: 520; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 4352 to 4395 of SEQ ID NO: 520; (f) a polyA sequence; and (g) a fifth polynucleotide sequence comprising nucleotides 4620 to 32350 of SEQ ID NO: 520.

Further disclosed herein are host cells comprising any of the polynucleotides, SAMs, expression cassettes, expression vectors, viral vectors, LNPs, PNPs, or nanoemulsions disclosed herein.

In some embodiments, the one or more polynucleotides are not integrated into the host cell genome, e.g., are episomal.

In some embodiments, the one or more polynucleotides are integrated into the host cell genome.

In some embodiments, the host cell is a mammalian cell.

In some embodiments, the mammalian cell is a human cell.

In some embodiments, the mammalian cell is not a human cell.

In some embodiments, the host cell is in vitro.

In some embodiments, the host cell is in vivo.

Further disclosed herein are polypeptides comprising any of the HIV immunogen amino acid sequences of SEQ ID NOs: 527-528.

Further disclosed herein are polypeptides comprising an amino acid sequence that is at least 80%, 85%, 86%, 87%, 88%, 89%, 90%, 91%, 92%, 93%, 94%, 95%, 96%, 97%, 98%, 99%, or 100% identical any of the HIV immunogen amino acid sequences of SEQ ID NOs: 527-528, wherein percent identity is across the full length of the HIV immunogen amino acid sequence of SEQ ID NOs: 527-528.

Further disclosed herein are compositions comprising (a) one or more of any of the polynucleotides, SAMs, expression cassettes, expression vectors, viral vectors, LNPs, PNPs, or nanoemulsions disclosed herein; and (b) a pharmaceutically acceptable diluent, carrier or excipient.

In some embodiments, the composition comprises two or more any of the polynucleotides, SAMs, expression cassettes, expression vectors, viral vectors, LNPs, PNPs, or nanoemulsions disclosed herein.

Further disclosed herein is a composition comprising (a) one or more the SAMs, expression cassettes, LNPs, PNPs, or nanoemulsions disclosed herein; and (b) one or more expression cassettes or viral vectors disclosed herein.

Further disclosed herein is a composition comprising (a) one or more polypeptides disclosed herein; and (b) a pharmaceutically acceptable diluent, carrier or excipient.

In some embodiments, the composition comprises two or more polypeptides disclosed herein.

In some embodiments, any of the compositions disclosed herein further comprise a pharmaceutically acceptable diluent, carrier or excipient.

In some embodiments, any of the compositions disclosed herein further comprise one or more of an adjuvant, an immunostimulator, a detergent, a micelle-forming agent, and an oil.

In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein.

In some embodiments, the cytokine selected from IL-2, IL-7, IL-12, IL-15, IL-18, IL-21, IFN-α, IFN-γ, GM-CSF, FLT3LG, and combinations and functional variants thereof.

In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102).

In some embodiments, the composition is formulated for administration via a route selected from the group consisting of intravenous, intramuscular, intradermal, subcutaneous and mucosal (e.g. buccal, intranasal, intrarectal, intravaginal).

In some embodiments, the composition is formulated as a liquid.

In some embodiments, the composition is lyophilized.

Further disclosed herein are kits comprising one or more components selected from one or more of the polynucleotides, SAMs, expression cassettes, expression vectors, viral vectors, LNPs, PNPs, nanoemulsions, or polypeptides disclosed herein.

Further disclosed herein are kits comprising (a) a first component selected from any of the SAMs, LNPs, PNPs, or nanoemulsions disclosed herein; and (b) second component comprising any of the expression vectors or viral vectors disclosed herein.

Further disclosed herein are kits comprising one or more unitary doses of any of the polynucleotides, SAMs, expression cassettes, expression vectors, viral vectors, LNPs, PNPs, nanoemulsions, or polypeptides disclosed herein.

Further disclosed herein are kits comprising (a) one or more unitary doses of any of the SAMs, LNPs, PNPs, or nanoemulsions disclosed herein; and (b) one or more unitary doses of any of the expression vectors or viral vectors disclosed herein.

Further disclosed herein are kits comprising one or more unitary doses of any of the polypeptides disclosed herein.

In some embodiments, the one or more components are in a single container.

In some embodiments, the one or more components are in are in two or more separate containers.

In some embodiments, the first and second components are in a single container.

In some embodiments, the first and second components are in are in two or more separate containers.

In some embodiments, the one or more unitary doses are in a single container.

In some embodiments, the one or more unitary doses are in two or more separate containers.

In some embodiments, the one or more containers are selected from the group consisting of vials, ampules and pre-loaded syringes.

In some embodiments, the one or more containers comprise the one or more SAMs, one or more LNPs, one or more PNPs, one or more nanoemulsions, one or more vectors, or one or more polypeptides in an aqueous solution.

In some embodiments, the one or more unitary doses are the same.

In some embodiments, the one or more unitary doses are the different.

In some embodiments, the unitary doses of the one or more viral vectors are in the range of about 10³ to about 10¹⁵ viral focus forming units (FFU) or plaque forming units (PFU) or infectious units (IU) or viral particles (vp).

In some embodiments, the unitary doses of the one or more viral vectors is from about 10⁴ to about 10⁷ viral FFU or PFU or IU or vp.

In some embodiments, the unitary doses of the one or more viral vectors is from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp.

In some embodiments, the unitary doses of the one or more SAMs are in the range of about 1 μg to about 1000 μg.

In some embodiments, the unitary doses of the one or more SAMs is from about 15 μg to about 500 μg.

In some embodiments, the unitary doses of the one or more SAMs is from about 1 μg to about 100, 200, 300, 400, 500, 600, 700, 800, 900, or 1000 μg.

In some embodiments, any of the compositions or kits disclosed herein further comprise one or more unitary doses of one or more additional therapeutic agents.

In some embodiments, the one or more additional therapeutic agents is selected from one or more agents that activate latent HIV, one or more agonists or activators of one or more toll-like receptors (TLRs), one or more interleukin receptor agonists, one or more cytokines, one or more receptor agonists, one or more inhibitors of a T-cell inhibitory immune checkpoint protein or receptor, one or more agonists, activators or stimulator of a T-cell stimulatory immune checkpoint protein or receptor, one or more inhibitors of CD47, one or more immune-based therapies, one or more broadly neutralizing antibodies (bnAbs), and one or more antiviral agents.

In some embodiments, the one or more agents that activate latent HIV is one or more latency reversing agents (LRAs).

In some embodiments, the one or more LRAs is selected from the group consisting of agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics.

In some embodiments, the TLR agonist or activator is selected from the group consisting of a TLR2 agonist, a TLR3 agonist, a TLR4 agonist, a TLR5 agonist, a TLR7 agonist, a TLR8 agonist and a TLR9 agonist.

In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist).

In some embodiments, any of the compositions or kits disclosed herein further comprise GS 9620 (vesatolimod).

In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042.

In some embodiments, any of the compositions or kits disclosed herein further comprise lefitolimod (MGN-1703).

In some embodiments, the interleukin is selected from IL-2, IL-7, IL-12, IL-15, IL-18, IL-21, IFN-a, IFN-g, GM-CSF, FLT3LG.

In some embodiments, the one or more cytokines selected from the group consisting of IL-2, IL-7, IL-12, IL-15, IL-18, IL-21, IFN-a, IFN-g, GM-CSF, FLT3LG, and combinations and functional variants thereof.

In some embodiments, the receptor agonist is an agonist of one or more receptors selected from fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2).

In some embodiments, the one or more inhibitors of a T-cell inhibitory immune checkpoint protein or receptor inhibits a checkpoint protein or receptor selected from the group consisting of CD274 (CD274, PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1).

In some embodiments, the one or more agonists, activators or stimulators of a T-cell stimulatory immune checkpoint protein or receptor agonizes, activates or stimulates a T-cell stimulatory immune checkpoint protein or receptor selected from the group consisting of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155).

In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002.

In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 ( ) JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181.

In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors.

Further disclosed herein are methods for eliciting an immune response to human immunodeficiency virus (HIV) in a subject in need thereof. In some embodiments, the method comprises administering to the subject one or more any of the polynucleotides, SAMs, LNPs, PNPs, nanoemulsions, expression cassettes, expression vectors, viral vectors, or polypeptides disclosed herein, or any compositions thereof.

Further disclosed herein are methods of treating or preventing human immunodeficiency virus (HIV) in a subject in need thereof. In some embodiments, the method comprises administering to the subject one or more any of the polynucleotides, SAMs, LNPs, PNPs, nanoemulsions, expression cassettes, expression vectors, viral vectors, or polypeptides disclosed herein, or any compositions thereof.

In some embodiments, the subject is infected with HIV-1, is suspected of being infected with HIV-1, or is at risk of being infected with HIV-1.

In some embodiments, the subject is chronically infected with HIV-1.

In some embodiments, the subject is acutely infected with HIV-1.

In some embodiments, the subject has an HIV-1 infection of Fiebig stage IV or earlier, e.g. Fiebig stage III, Fiebig stage II or Fiebig stage I.

In some embodiments, administering comprises a route of administration selected from intravenous, intramuscular, intradermal, subcutaneous and mucosal (e.g. buccal, intranasal, intrarectal, intravaginal).

In some embodiments, the method comprises administering one or more viral vectors of at a dose range from about 10³ to about 10¹⁵ viral focus forming units (FFU) or plaque forming units (PFU) or infectious units (IU) or viral particles (vp), per administration.

In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration.

In some embodiments, the method comprises administering one or more the SAMs, LNPs, PNPs, or nanoemulsions disclosed herein at a dose range from about 1 μg to about 1000 ug.

In some embodiments, the one or more SAMs, LNPs, PNPs, or nanoemulsions is administered at a dose range from about 15 μg to about 500 μg.

In some embodiments, the one or more SAMs, LNPs, PNPs, or nanoemulsions is administered at a dose range from about 1 μg to about 100, 200, 300, 400, 500, 600, 700, 800, 900, or 1000 μg.

In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.).

In some embodiments, the administrations of the priming composition and the one or more boosting compositions are spaced at least 1 week, 2 weeks, 3 weeks or 1 month apart, e.g., at least 2, 3, 4, 5 or 6 months, apart.

In some embodiments, the priming composition and the boosting composition comprise a polynucleotide encoding the same immunogenic polypeptide.

In some embodiments, the priming composition and the boosting composition comprise polynucleotide encoding the different immunogenic polypeptides.

In some embodiments, the priming composition and the boosting composition comprise the same one or more polypeptides, polynucleotides, SAMs, LNPs, PNPs, nanoemulsions, or viral vectors.

In some embodiments, the priming composition and the boosting composition comprise different polypeptides, polynucleotides, SAMs, LNPs, PNPs, nanoemulsions, or viral vectors.

In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector.

In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM.

In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors.

In some embodiments, ART is discontinued after one or more administrations of the polynucleotides, polypeptides, SAMs, LNPs, PNPs, nanoemulsions, expression cassettes, or viral vectors.

In some embodiments, any of the methods disclosed herein further comprise administering to the subject one or more additional therapeutic agents, e.g. two, three, four, or more additional therapeutic agents.

In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs).

In some embodiments, the one or more agents that activate latent HIV is one or more latency reversing agents (LRAs).

In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics.

In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist.

In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist).

In some embodiments, any of the methods disclosed herein further comprise administering GS 9620 (vesatolimod) to the subject.

In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042.

In some embodiments, any of the methods disclosed herein further comprise administering lefitolimod (MGN-1703) to the subject.

In some embodiments, the one or more bnAbs is an HIV bnAb.

In some embodiments, any of the methods disclosed herein further comprise administering one or more bnAbs.

In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses.

In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120.

In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/I1-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13.

In some embodiments, the bnAb is an anti-CD4 bnAb.

In some embodiments, the anti-CD4 bnAb is selected from 3BNC117 and VRC01.

In some embodiments, the bnAb is an anti-V3 bnAb.

In some embodiments, the anti-V3 bnAb is 10-1074.

In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG.

In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof.

In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2).

In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1).

In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155).

In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002.

In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181.

In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors.

In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more.

In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein is the use of any of the polynucleotides, SAMs, expression cassettes, LNPs, PNPs, nanoemulsions, expression vectors, viral vectors, polypeptides, or compositions disclosed herein for eliciting an immune response to human immunodeficiency virus (HIV) in a subject in need thereof.

Further disclosed herein is the use of any of the polynucleotides, SAMs, expression cassettes, LNPs, PNPs, nanoemulsions, expression vectors, viral vectors, polypeptides, or compositions disclosed herein for treating or preventing human immunodeficiency virus (HIV) in a subject in need thereof.

Further disclosed herein is the use of any of the polynucleotides, SAMs, expression cassettes, LNPs, PNPs, nanoemulsions, expression vectors, viral vectors, polypeptides, or compositions disclosed herein as a medicament.

### BRIEF DESCRIPTION OF THE DRAWINGS

FIG. 1 illustrates an 8-step workflow for designing a fusion polypeptide to elicit an antiviral response.

FIG. 2 illustrates a representative methodology of a population-based vaccine construct approach.

FIG. 3 illustrates the steps of the conserved walking analysis (CWA) algorithm, as described herein.

FIGS. 4A-4B. FIG. 4A illustrates how “bivalent conservation” can be determined based on the prevalence of the two most common 9-mers among all considered viral sequences in a population. FIG. 4A discloses SEQ ID NOS 475-476, 476-477, 476, 476, 478-479 and 479-480 in the top panel, respectively, in order of appearance. FIG. 4A also discloses “QNLQGQMVH” as SEQ ID NO: 481, “QNIQGQMVH” as SEQ ID NO: 482 and “PNIQGQMVH” as SEQ ID NO: 483 in the bottom panel. FIG. 4B illustrates how the conserved regions are identified based on the “bivalent conservation” distribution across 9-mer positions. HIV-1 Gag p24 was used as the representative protein.

FIGS. 5A-5C. FIG. 5A illustrates unique 9-mers extracted from aligned natural sequences. FIG. 5B illustrates a directed acyclic graph built based on 9-mer pair nodes and their connection. FIG. 5C illustrates how 9-mers in connected 9-mer pairs are connected. When there are two options available for the connection, the ultimate connection is determined by the prevalence of each connection in naturally occurring sequences. FIGS. 5A-5C disclose “IIIIIIIIR” as SEQ ID NO: 467, “GIIIIIIIIH” as SEQ ID NO: 473, “AIIIIIIIIK” as SEQ ID NO: 474, “GIIIIIIIIR” as SEQ ID NO: 484, “GIIIIIIII” as SEQ ID NO: 485, “AIIIIIIII” as SEQ ID NO: 486, “IIIIIIIIK” as SEQ ID NO: 487 and “IIIIIIIIH” as SEQ ID NO: 488.

FIG. 6 illustrates the results of human proteome cross-recognition analysis. FIG. 6 discloses the “HIV Peptide” sequence as SEQ ID NO: 492 and the “Human Protein 9-mer” sequences as SEQ ID NOS 493-498 and 493, respectively, in order of appearance.

FIG. 7 illustrates how polypeptide segment arrangement analysis can reduce or eliminate possible presentation of deleterious or undesirable epitopes in junction regions.

FIG. 8 illustrates rational antiviral immunogen design approach that considers for interpatient and intrapatient viral sequence diversity and host MHC class I and class II molecules binding, and T cell recognition. The approach and resulting immunogens are exemplified herein by immunogens that elicit human immune responses against HIV-1.

FIG. 9 shows a schematic of antigen-specific T cell responses in mice vaccinated with ChAd-01, ChAd-02, SAM-01, SAM-02 (corresponding to SEQ ID NOs: 520-523, respectively) or PBS control.

FIG. 10A-10D shows a schematic of antigen-specific T cell responses in mice vaccinated with ChAd-01, ChAd-02, SAM-01, SAM-02, or PBS control. FIG. 10A illustrates T cell responses to Pol-1 in mice vaccinated with ChAd-01, ChAd-02, SAM-01, SAM-02, or PBS control. FIG. 10B illustrates T cell responses to Pol-2 in mice vaccinated with ChAd-01, ChAd-02, SAM-01, SAM-02, or PBS control. FIG. 10C illustrates T cell responses to Gag in mice vaccinated with ChAd-01, ChAd-02, SAM-01, SAM-02, or PBS control. FIG. 10D illustrates T cell responses to Nef in mice vaccinated with ChAd-01, ChAd-02, SAM-01, SAM-02, or PBS control.

FIG. 11 shows a schematic diagram of the design, procedures, and treatments for a clinical trial.

FIG. 12 shows a schematic of fusion polypeptides referred to as Immunogen 1A and Immunogen 1B, which correspond to the amino acid sequences of SEQ ID NOs: 527-528, respectively. The start and end sites of the peptide segments for Gag, Pol, and Nef are indicated in the arrangement details.

FIG. 13 shows SIV-specific T cell response in rhesus macaques following vaccination with ChAd/samRNA alone (circle) or in combination with anti-PD-1 antibody (square), anti-CTLA4 antibody (triangle) or FLT3R agonist (upside-down triangle). IFNγ ELISpot, sum of Env, Pol and Gag overlapping peptide pools, background subtracted. Mean±SEM. Black arrows represents ChAd immunizations and grey arrows represent samRNA immunizations.

FIG. 14A shows a linear schematic of a ChAd vector comprising a CMV promoter, immunogen, and polyA.

FIG. 14B shows a linear schematic of a samRNA vector comprising a 26S subgenomic promoter, immunogen, and polyA.

FIG. 15 shows the breadth of SIV Gag-specific IFNγ T cell responses in PBMCs from rhesus macaques following vaccination with ChAd/samRNA alone or in combination with anti-PD-1 antibody (aPD1), anti-CTLA4 antibody (aCTLA4) or FLT3R agonist (FLT3Ra).

FIG. 16 shows SIV-specific CD8+ and CD4+ T-cell populations in rhesus macaques following vaccination with ChAd/samRNA alone or in combination with anti-PD-1 antibody (aPD1), anti-CTLA4 antibody (aCTLA4) or FLT3R agonist (FLT3Ra) measured by intracellular cytokine staining of PBMCs at week 14 following overnight stimulation with 4 overlapping peptide pools spanning Gag, Pol, and Env background subtracted; sum of pools presented as median (IQR).

FIG. 17 shows functionality of SIV-specific T-cells in rhesus macaques following vaccination with ChAd/samRNA alone or in combination with anti-PD-1 antibody (aPD1), anti-CTLA4 antibody (aCTLA4) or FLT3R agonist (FLT3Ra) measured by the intracellular cytokine staining of PBMCs at week 14 following overnight stimulation with 4 overlapping peptide pools spanning Gag, Pol, and Env background subtracted.

FIG. 18 shows serum exposure levels of aPD-1, aCTLA4, and Flt3Ra in rhesus macaques.

FIG. 19 shows PD-1 receptor occupancy in rhesus macaques following vaccination with ChAd/samRNA in combination with anti-PD-1 antibody (aPD1) administered by SC or IV route compared to vaccination alone.

FIG. 20 shows cDC1 cell expansion over time from baseline in PBMCs of rhesus macaques dosed with FLT3R agonist (FLT3Ra) followed by vaccination with ChAd/samRNA.

### DETAILED DESCRIPTION

Provided herein are HIV immunogenic polypeptides. In some embodiments, the HIV immunogenic polypeptides comprise the amino acid sequence of SEQ ID NOs: 527-528.

Further provided herein are polynucleotides encoding HIV immunogenic polypeptides. In some embodiments, the polynucleotides comprise a nucleic acid sequence that is at least 80%, 85%, 86%, 87%, 88%, 89%, 90%, 91%, 92%, 93%, 94%, 95%, 96%, 97%, 98%, 99%, or 100% identical to any one of the HIV immunogen nucleic acid sequences of SEQ ID NOs: 524-526, wherein percent identity is across the full length of the HIV immunogen nucleic acid sequence of SEQ ID NOs: 524-526. In some embodiments, the polynucleotides comprise a nucleic acid sequence that encodes any one of the HIV immunogen amino acid sequences of SEQ ID NOS: 527-528. In some embodiments, the polynucleotides comprise a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 527-528, wherein percent identity is across the full length of the nucleic acid sequence of the polypeptide encoding any one of the HIV immunogen amino acid sequence of SEQ ID NOs: 527-528.

Further disclosed herein are expression cassettes comprising a polynucleotide that encodes for any of the HIV immunogenic polypeptides disclosed herein. In some embodiments, the expression cassette comprises a polynucleotide that encodes for any of the HIV immunogenic polypeptides of SEQ ID NOs: 1-371, 373-377, 407-411, 422-423, 430-435, 527, and 528. In some embodiments, the expression cassette comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 524-526.

Further disclosed herein are expression vectors comprising a polynucleotide that encodes for any of the HIV immunogenic polypeptides disclosed herein. In some embodiments, the expression vector comprises a polynucleotide that encodes for any of the HIV immunogenic polypeptides of SEQ ID NOs: 1-371, 373-377, 407-411, 422-423, 430-435, 527, and 528. In some embodiments, the expression vector comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 520-521. In some embodiments, the expression vector comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 522-523. In some embodiments, the expression vector comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 524-526.

Further disclosed herein are self-amplifying mRNAs (SAMs) comprising a polynucleotide that encodes for any of the HIV immunogenic polypeptides disclosed herein. In some embodiments, the SAM comprises a polynucleotide that encodes for any of the HIV immunogenic polypeptides of SEQ ID NOs: 1-371, 373-377, 407-411, 422-423, 430-435, 527, and 528. In some embodiments, the SAM comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 522-523. In some embodiments, the SAM comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 524-526.

Further disclosed herein are viral vectors comprising a polynucleotide that encodes for any of the HIV immunogenic polypeptides disclosed herein. In some embodiments, the viral vector comprises a polynucleotide that encodes for any of the HIV immunogenic polypeptides of SEQ ID NOs: 1-371, 373-377, 407-411, 422-423, 430-435, 527, and 528. In some embodiments, the viral vector comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 520-521. In some embodiments, the viral vector comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 524-526.

Further disclosed herein are lipid nanoparticles (LNPs), polymeric nanoparticles (PNPs), or nanoemulsions comprising any of the polynucleotides, expression cassettes, expression vectors, or viral vectors disclosed herein.

Further disclosed herein are cells comprising any of the polypeptides, polynucleotides, expression cassettes, expression vectors, viral vectors, LNPs, PNPs, or nanoemulsions disclosed herein.

Further disclosed herein are compositions comprising any of the polypeptides, polynucleotides, expression cassettes, expression vectors, viral vectors, LNPs, PNPs, or nanoemulsions disclosed herein.

Further disclosed herein are kits comprising any of the polypeptides, polynucleotides, expression cassettes, expression vectors, viral vectors, LNPs, PNPs, or nanoemulsions disclosed herein.

Further disclosed herein are methods of using any of the polypeptides, polynucleotides, expression cassettes, expression vectors, viral vectors, LNPs, PNPs, or nanoemulsions disclosed herein for eliciting an immune response to human immunodeficiency virus (HIV) in a subject in need thereof.

Further disclosed herein are methods of using any of the polypeptides, polynucleotides, expression cassettes, expression vectors, viral vectors, LNPs, PNPs, or nanoemulsions disclosed herein for treating or preventing human immunodeficiency virus (HIV) in a subject in need thereof.

Further disclosed herein are methods of using any of the polypeptides, polynucleotides, expression cassettes, expression vectors, viral vectors, LNPs, PNPs, or nanoemulsions disclosed herein for treating or preventing human immunodeficiency virus (HIV) in a subject in need thereof.

### HIV Immunogenic Polypeptides

Disclosed herein are HIV immunogenic polypeptides. As used herein, the phrase “HIV immunogenic polypeptide” refers to a polypeptide that can elicit an immunogenic response to a human immunodeficiency virus (HIV-1) in a subject in need thereof. As used herein, an “immunogen” is a substance, such as an antigen, that elicits an immune response or is capable of eliciting an immune response. The phrases “HIV immunogenic polypeptide” and “fusion polypeptide” may be used interchangeably.

### Polypeptide Segments

The HIV immunogenic polypeptides comprise a plurality of polypeptide or peptide segments encoded by one or more HIV-1 genes. A ‘segment’ of an HIV immunogenic polypeptide (e.g., fusion polypeptide) described herein is a contiguous sequence of at least 8 amino acids with respect to a reference sequence, for example HIV-1 HXB2 reference sequences for Env, Gag, Nef and Pol polypeptides, provided herein as SEQ ID NOs: 403-406, respectively. The HIV immunogenic polypeptides described herein may be described as ‘fusion’ polypeptides in the sense that they are assembled from connected or concatenated polypeptide or peptide segments of two or more HIV-1 proteins. With respect to the HIV-1 protein reference sequences, the polypeptide or peptide segments may correspond to discontinuous sequences of the same HIV-1 protein or different HIV-1 proteins. Generally, the HIV immunogenic polypeptide (e.g., fusion polypeptides) are non-naturally occurring, and can be synthetic or recombinantly produced.

With respect to the HIV-1 genes encoding the polypeptide segments used to assemble the herein described fusion polypeptides, in various embodiments, the fusion polypeptides comprise a plurality of polypeptide segments of one or more human immunodeficiency virus-1 (HIV-1) proteins encoded by one or more, e.g. two or more, three or more, four or more, HIV-1 genes selected from Gag, Nef, Env, Pol, Vpu, Vpr and Vif, e.g., two or more, three or more, four or more, HIV-1 genes selected from Gag, Nef, Env, Pol, and Vif. In some embodiments, the plurality of polypeptide segments is comprised of only polypeptide segments encoded by HIV-1 genes Env, Gag, Nef and Pol, e.g., does not comprise polypeptide segments encoded by HIV-1 Tat, Rev, Vif, Vpr and/or Vpu genes. In some embodiments, the plurality of polypeptide segments is comprised of only polypeptide segments encoded by HIV-1 genes Gag, Nef and Pol, e.g., does not comprise polypeptide segments encoded by HIV-1 Env, Tat, Rev, Vif, Vpr and/or Vpu genes. In some embodiments, the plurality of polypeptide segments is comprised of only polypeptide segments encoded by HIV-1 genes Gag and Nef, e.g., does not comprise polypeptide segments encoded by HIV-1 Env, Pol, Tat, Rev, Vif, Vpr and/or Vpu genes. In some embodiments, the plurality of polypeptide segments is comprised of only polypeptide segments encoded by HIV-1 genes Pol and Nef, e.g., does not comprise polypeptide segments encoded by HIV-1 Env, Gag, Tat, Rev, Vif, Vpr and/or Vpu genes. In some embodiments, the plurality of polypeptide segments is comprised of only polypeptide segments encoded by HIV-1 genes Pol and Env, e.g., does not comprise polypeptide segments encoded by HIV-1 Gag, Nef, Tat, Rev, Vif, Vpr and/or Vpu genes. In some embodiments, the plurality of polypeptide segments is comprised of only polypeptide segments encoded by HIV-1 Pol gene, e.g., does not comprise polypeptide segments encoded by HIV-1 Env, Gag, Nef, Tat, Rev, Vif, Vpr and/or Vpu genes. In various embodiments, the plurality of polypeptide segments does not contain a segment encoded by one, two, three or four of HIV Tat, Rev, Vif, Vpr and/or Vpu genes.

With respect to the number of polypeptide segments assembled, connected, linked or concatenated into a single fusion polypeptide, in various embodiments, the fusion polypeptides are comprised of at least 5 and up to 40 polypeptide segments, e.g., from 5 polypeptide segments and up to 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39 or 40 polypeptide segments. As appropriate, the polypeptide segments can be arranged in the same order or according to a different order than in the naturally occurring proteins.

With respect to the regions of the polypeptides encoded by an HIV-1 gene selected as polypeptide segments to include in the fusion polypeptides, in various embodiments, the polypeptide segments are derived from conserved regions in a population of viral proteome sequences. In some embodiments, the conserved regions are greater than 80%, e.g., greater than 81%, 82%, 83%, 84%, 85%, 86%, 87%, 88%, 89%, 90%, 91%, 92%, 93%, 94%, 95%, 96%, 97%, 98% or 99% conserved amongst HIV-1 species, e.g., as determined in interpatient and/or intrapatient populations. As used herein, conserved regions in a polypeptide encoded by an HIV-1 gene refers to the percentage of sequences in a population of sequences containing identical amino acid segments or subsequences e.g., (segments 9 amino acids in length or 9-mers) as the most prevalent one in a predetermined amino acid segment or subsequence position, where an amino acid segment or subsequence position is determined with respect to a reference sequence, e.g., HIV-1 HXB2 polypeptide sequences, e.g., SEQ ID NOs: 403-406. In various embodiments, the conserved regions are conserved amongst one or more of HIV-1 clades within Group M, e.g., one or more of HIV-1 clades A-K, e.g., one or more of clades A, B, C, D and G, e.g., amongst HIV-1 Group M, clade B, and recombinant forms thereof, e.g., CRF01_AE. In some embodiments, the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344, e.g., polypeptide segments identified in Table 2. In some embodiments, the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 2, 3, 8, 9, 13, 14, 17, 18, 23, 24, 25, 26, 28, 29, 31, 32, 33, 34, 35, 36, 37, 38, 39, 40, 43, 44, 45, 46, 47, 48, 49, 50, 51, 52, 53, 54, 55, 56, 58, 59, 62, 63, 64, 65, 66, 67, 68, 69, 72, 73, 74, 75, 76, 77, 80, 81, 82, 83, 84, 85, 86, 87, 88, 89, 92, 93, 101, 102, 103, 104, 109, 110, 115, 116, 125, 126, 127, 128, 129, 130, 131, 132, 133, 134, 135, 136, 139, 140, 141, 142, 143, 144, 145, 146, 147, 148, 149, 150, 155, 156, 157, 158, 159, 160, 166, 167, 168, 169, 170, 171, 174, 175, 178, 179, 180, 181, 182, 183, 184, 185, 193, 194, 195, 196, 197, 198, 199, 200, 203, 204, 205, 206, 207, 208, 213, 214, 221, 222, 236, 237, 245, 246, 247, 248, 249, 250, 251, 252, 253, 254, 255, 256, 257, 258, 263, 264, 266, 267, 268, 269, 270, 271, 272, 273, 278, 279, 280, 281, 282, 283, 284, 285, 286, 287, 288, 289, 290, 291, 292, 293, 294, 295, 305, 306, 307, 308, 309, 310, 313, 314, 315, 316, 317, 318, 321 and 322, e.g., polypeptide segments identified in Table 3. The start and end positions are with respect to HIV-1 HXB2 reference polypeptides, GenBank Accession No. K03455 (ncbi.nlm.nih.gov/nuccore/K03455), provided herein as SEQ ID NOs: 403-406 and identified in Table 1.

TABLE 1
HIV-1 HXB2 reference sequences

| SEQ | | |
|---|---|---|
| ID NO: | GENE | SEQUENCE |
| 403 | Env | MRVKEKYQHLWRWGWRWGTMLLGMLMICSATEKLWVTVYYGVPVWKE |
| | | ATTTLFCASDAKAYDTEVHNVWATHACVPTDPNPQEVVLVNVTENFNMW |
| | | KNDMVEQMHEDIISLWDQSLKPCVKLTPLCVSLKCTDLKNDTNTNSSSGRMIM |
| | | EKGEIKNCSFNISTSIRGKVQKEYAFFYKLDIIPIDNDTTSYKLTSCNTSVIT |
| | | QACPKVSFEPIPIHYCAPAGFAILKCNNKTFNGTGPCTNVSTVQCTHGIRPVV |
| | | STQLLLNGSLAEEEVVIRSVNFTDNAKTIIVQLNTSVEINCTRPNNNTRKRIRI |
| | | QRGPGRAFVTIGKIGNMRQAHCNISRAKWNNTLKQIASKLREQFGNNKTIIF |
| | | KQSSGGDPEIVTHSFNCGGEFFYCNSTQLFNSTWFNSTWSTEGSNNTEGSDTI |
| | | TLPCRIKQIINMWQKVGKAMYAPPISGQIRCSSNITGLLLTRDGGNSNNESEI |
| | | FRPGGGDMRDNWRSELYKYKVVKIEPLGVAPTKAKRRVVQREKRAVGIGAL |
| | | FLGFLGAAGSTMGAASMTLTVQARQLLSGIVQQQNNLLRAIEAQQHLLQLT |
| | | VWGIKQLQARILAVERYLKDQQLLGIWGCSGKLICTTAVPWNASWSNKSLE |
| | | QIWNHTTWMEWDREINNYTSLIHSLIEESQNQQEKNEQELLELDKWASLWN |
| | | WFNITNWLWYIKLFIMIVGGLVGLRIVFAVLSIVNRVRQGYSPLSFQTHLPTP |
| | | RGPDRPEGIEEEGGERDRDRSIRLVNGSLALIWDDLRSLCLFSYHRLRDLLLI |
| | | VTRIVELLGRRGWEALKYWWNLLQYWSQELKNSAVSLLNATAIAVAEGTD |
| | | RVIEVVQGACRAIRHIPRRIRQGLERILL |
| 404 | Gag | MGARASVLSGGELDRWEKIRLRPGGKKKYKLKHIVWASRELERFAVNPGLL |
| | | ETSEGCRQILGQLQPSLQTGSEELRSLYNTVATLYCVHQRIEIKDTKEALDK |
| | | IEEEQNKSKKKAQQAAADTGHSNQVSQNYPIVQNIQGQMVHQAISPRTLNA |
| | | WVKVVEEKAFSPEVIPMFSALSEGATPQDLNTMLNTVGGHQAAMQMLKETI |
| | | NEEAAEWDRVHPVHAGPIAPGQMREPRGSDIAGTTSTLQEQIGWMTNNPPIP |
| | | VGEIYKRWIILGLNKIVRMYSPTSILDIRQGPKEPFRDYVDRFYKTLRAEQA |
| | | SQEVKNWMTETLLVQNANPDCKTILKALGPAATLEEMMTACQGVGGPGHK |
| | | ARVLAEAMSQVTNSATIMMQRGNFRNQRKIVKCFNCGKEGHTARNCRAPR |
| | | KKGCWKCGKEGHQMKDCTERQANFLGKIWPSYKGRPGNFLQSRPEPTAPPE |
| | | ESFRSGVETTTPPQKQEPIDKELYPLTSLRSLFGNDPSSQ |
| 405 | Nef | MGGKWSKSSVIGWPTVRERMRRAEPAADRVGAASRDLEKHGAITSSNTAA |
| | | TNAACAWLEAQEEEEVGFPVTPQVPLRPMTYKAAVDLSHFLKEKGGLEGL |
| | | IHSQRRQDILDLWIYHTQGYFPDWQNYTPGPGVRYPLTFGWCYKLVPVEP |
| | | DKIEEANKGENTSLLHPVSLHGMDDPEREVLEWRFDSRLAFHHVARELHP |
| | | EYFKNC |
| 406 | Pol | FFREDLAFLQGKAREFSSEQTRANSPTRRELQVWGRDNNSPSEAGADRQGT |
| | | VSFNFPQVTLWQRPLVTIKIGGQLKEALLDTGADDTVLEEMSLPGRWKPKMIG |
| | | GIGGFIKVRQYDQILIEICGHKAIGTVLVGPTPVNIIGRNLLTQIGCTLNFPI |
| | | SPIETVPVKLKPGMDGPKVKQWPLTEEKIKALVEICTEMEKEGKISKIGPENP |
| | | YNTPVFAIKKKDSTKWRKLVDFRELNKRTQDFWEVQLGIPHPAGLKKKKSVTV |
| | | LDVGDAYFSVPLDEDFRKYTAFTIPSINNETPGIRYQYNVLPQGWKGSPAIFQ |
| | | SSMTKILEPFRKQNPDIVIYQYMDDLYVGSDLEIGQHRTKIEELRQHLLRWG |
| | | LTTPDKKHQKEPPFLWMGYELHPDKWTVQPIVLPEKDSWTVNDIQKLVGKL |
| | | NWASQIYPGIKVRQLCKLLRGTKALTEVIPLTEEAELELAENREILKEPVHGV |
| | | YYDPSKDLIAEIQKQGQGQWTYQIYQEPFKNLKTGKYARMRGAHTNDVKQ |
| | | LTEAVQKITTESIVIWGKTPKFKLPIQKETWETWWTEYWQATWIPEWEFVNT |
| | | PPLVKLWYQLEKEPIVGAETFYVDGAANRETKLGKAGYVTNRGRQKVVTLTD |
| | | TTNQKTELQAIYLALQDSGLEVNIVTDSQYALGIIQAQPDQSESELVNQIIE |
| | | QLIKKEKVYLAWVPAHKGIGGNEQVDKLVSAGIRKVLFLDGIDKAQDEHEK |
| | | YHSNWRAMASDFNLPPVVAKEIVASCDKCQLKGEAMHGQVDCSPGIWQLD |
| | | CTHLEGKVILVAVHVASGYIEAEVIPAETGQETAYFLLKLAGRWPVKTIHTD |
| | | NGSNFTGATVRAACWWAGIKQEFGIPYNPQSQGVVESMNKELKKIIGQVRD |
| | | QAEHLKTAVQMAVFIHNFKRKGGIGGYSAGERIVDIIATDIQTKELQKQITKI |
| | | QNFRVYYRDSRNPLWKGPAKLLWKGEGAVVIQDNSDIKVVPRRKAKIIRDY |
| | | GKQMAGDDCVASRQDED |

TABLE 2
all polypeptide segments

| SEQ ID | | | | |
|---|---|---|---|---|
| NO | Gene | Start | End | Sequence |
| 1 | Env | 28 | 52 | CSATEKLWVTVYYGVPVWKEATTTL |
| 2 | Env | 34 | 48 | LWVTVYYGVPVWKEA |
| 3 | Env | 34 | 48 | LWVTIYYGVPVWKDA |
| 4 | Env | 34 | 47 | LWVTVYYGVPVWKE |
| 5 | Env | 34 | 47 | LWVTIYYGVPVWKD |
| 6 | Env | 36 | 44 | VTVYYGVPV |
| 7 | Env | 36 | 44 | VTIYYGVPV |
| 8 | Env | 48 | 61 | ATTTLFCASDAKAY |
| 9 | Env | 48 | 61 | ANTTLFCASDAKGY |
| 10 | Env | 59 | 83 | KAYDTEVHNVWATHACVPTDPNPQE |
| 11 | Env | 64 | 83 | AHNVWATHACVPTDPNPQE |
| 12 | Env | 64 | 83 | VHNIWATHACVPTDPSPQE |
| 13 | Env | 65 | 83 | HNVWATHACVPTDPNPQE |
| 14 | Env | 65 | 83 | HNIWATHACVPTDPSPQE |
| 15 | Env | 67 | 75 | NVWATHACV |
| 16 | Env | 67 | 75 | NIWATHACV |
| 17 | Env | 107 | 129 | DIISLWDQSLKPCVKLTPLCVTL |
| 18 | Env | 107 | 129 | DIISLWDESLKPCVKLTPICVTL |
| 19 | Env | 113 | 137 | DQSLKPCVKLTPLCVTLNCTDLRNT |
| 20 | Env | 113 | 137 | DESLKPCVKLTPICVTLNCTDLRNT |
| 21 | Env | 121 | 129 | KLTPLCVTL |
| 22 | Env | 121 | 129 | KLTPICVTL |
| 23 | Env | 209 | 226 | SFEPIPIHYCAPAGFAIL |
| 24 | Env | 209 | 226 | TFEPIPIHYCTPAGFAIL |
| 25 | Env | 220 | 228 | PAGFAILKC |
| 26 | Env | 220 | 228 | PAGFALLKC |
| 27 | Env | 235 | 259 | GTGPCTNVSTVQCTHGIRPVVSTQL |
| 28 | Env | 241 | 268 | NVSTVQCTHGIRPVVSTQLLLNGSLAEE |
| 29 | Env | 241 | 268 | NISTVQCTHGIKPVVSTQLLLNGSLAEK |
| 30 | Env | 243 | 251 | STVQCTHGI |
| 31 | Env | 376 | 386 | FNCGGEFFYCN |
| 32 | Env | 376 | 386 | FNCRGEFFYCN |
| 33 | Env | 430 | 439 | VGKAMYAPPI |
| 34 | Env | 430 | 439 | VGRAMYAPPI |
| 35 | Env | 472 | 481 | GGDMRDNWRS |
| 36 | Env | 472 | 481 | GGNMKDNWRS |
| 37 | Env | 475 | 489 | MRDNWRSELYKYKVV |
| 38 | Env | 475 | 489 | MKDNWRSELYRYKVV |
| 39 | Env | 501 | 511 | AKRRVVQREKR |
| 40 | Env | 501 | 511 | ARRRVVQREKR |
| 41 | Env | 502 | 606 | KRRVVQREKRAVGIGAMFLGFLGAAGSTMG |
| | | | | AASITLTVQARQLLSGIVQQQNNLLRAIEAQ |
| | | | | QHLLQLTVWGIKQLQARVLAVERYLKDQQL |
| | | | | LGIWGCSGKLICTT |
| 42 | Env | 502 | 606 | RRRVVQREKRAIGLGAVFLGFLGTAGSTMG |
| | | | | AASMTLTVQARLLLSGIVQQQSNLLRAIEAQ |
| | | | | QHMLQLTVWGIKQLQARILAVERYLRDQQL |
| | | | | LGIWGCSGRLICTT |
| 43 | Env | 519 | 534 | FLGFLGAAGSTMGAAS |
| 44 | Env | 519 | 534 | FLGFLGTAGSTMGAAA |
| 45 | Env | 533 | 606 | ASITLTVQARQLLSGIVQQQNNLLRAIEAQQ |
| | | | | HLLQLTVWGIKQLQARVLAVERYLKDQQLL |
| | | | | GIWGCSGKLICTT |
| 46 | Env | 533 | 606 | ASMTLTVQARLLLSGIVQQQSNLLRAIEAQQ |
| | | | | HMLQLTVWGIKQLQARILAVERYLRDQQLL |
| | | | | GIWGCSGRLICTT |
| 47 | Env | 536 | 556 | TLTVQARQLLSGIVQQQNNLL |
| 48 | Env | 536 | 556 | TLTVQARLLLSGIVQQQSNLL |
| 49 | Env | 554 | 564 | NLLRAIEAQQH |
| 50 | Env | 554 | 564 | NLLKAIEAQQH |
| 51 | Env | 558 | 584 | AIEAQQHLLQLTVWGIKQLQARVLAVE |
| 52 | Env | 558 | 584 | AIEAQQHMLQLTVWGIKQLQARILAVE |
| 53 | Env | 584 | 592 | ERYLKDQQL |
| 54 | Env | 584 | 592 | ERYLRDQQL |
| 55 | Env | 586 | 594 | YLKDQQLLG |
| 56 | Env | 586 | 594 | YLRDQQLLG |
| 57 | Env | 586 | 610 | YLKDQQLLGIWGCSGKLICTTAVPW |
| 338 | Env | 586 | 610 | YLRDQQLLGLWGCSGKLICPTAVPW |
| 58 | Env | 589 | 606 | DQQLLGIWGCSGKLICTT |
| 59 | Env | 589 | 606 | DQQLLGLWGCSGKLICPT |
| 60 | Env | 594 | 602 | GIWGCSGKL |
| 61 | Env | 594 | 602 | GLWGCSGKL |
| 62 | Env | 678 | 688 | WLWYIKIFIMI |
| 63 | Env | 678 | 688 | WLWYIRIFIMI |
| 64 | Env | 684 | 697 | IFIMIVGGLIGLRI |
| 65 | Env | 684 | 697 | LFIMIVGGLVGLRI |
| 66 | Env | 705 | 719 | VNRVRQGYSPLSFQT |
| 67 | Env | 705 | 719 | VNRVRKGYSPLSFQI |
| 68 | Gag | 1 | 11 | MGARASVLSGG |
| 69 | Gag | 1 | 11 | MGARASILSGG |
| 70 | Gag | 1 | 53 | MGARASVLSGGELDRWEKIRLRPGGKKKYR |
| | | | | LKHIVWASRELERFAVNPGLLET |
| 71 | Gag | 1 | 53 | MGARASILSGGKLDKWEKIRLRPGGRKKYK |
| | | | | LKHIVWASRELERFAVNPGLLET |
| 72 | Gag | 13 | 25 | LDRWEKIRLRPGG |
| 73 | Gag | 13 | 25 | LDKWEKIRLRPMG |
| 74 | Gag | 19 | 27 | IRLRPGGKK |
| 75 | Gag | 19 | 27 | IRLRPGGRK |
| 76 | Gag | 31 | 53 | LKHIVWASRELERFAVNPGLLET |
| 77 | Gag | 31 | 53 | LKHLVWASRELERFALNPGLLET |
| 78 | Gag | 37 | 51 | ASRELERFAVNPGLL |
| 79 | Gag | 37 | 51 | ASRELERFALNPGLL |
| 80 | Gag | 70 | 78 | TGSEELKSL |
| 81 | Gag | 70 | 78 | TGSEELRSL |
| 82 | Gag | 96 | 104 | DTKEALDKI |
| 83 | Gag | 96 | 104 | DTKEALEKI |
| 84 | Gag | 99 | 107 | EALDKIEEE |
| 85 | Gag | 99 | 107 | EALEKIEEE |
| 86 | Gag | 128 | 137 | VSQNYPIVQN |
| 87 | Gag | 128 | 137 | VSQNFPIVQN |
| 88 | Gag | 133 | 363 | PIVQNLQGQMVHQAISPRTLNAWVKVVEEK |
| | | | | AFSPEVIPMFSALSEGATPQDLNTMLNTVGG |
| | | | | HQAAMQMLKETINEEAAEWDRLHPVHAGPI |
| | | | | APGQMREPRGSDIAGTTSTLQEQIGWMTNNP |
| | | | | PIPVGEIYKRWIILGLNKIVRMYSPTSILDIR |
| | | | | QGPKEPFRDYVDRFYKTLRAEQASQEVKNWM |
| | | | | TETLLVQNANPDCKTILKALGPAATLEEMMT |
| | | | | ACQGVGGPGHKARVL |
| 89 | Gag | 133 | 363 | PIVQNIQGQMVHQPISPRTLNAWVKVIEEKA |
| | | | | FSPEVIPMFTALSEGATPHDLNTMLNTIGGHQ |
| | | | | AAMQMLKDTINEEAAEWDRVHPVHAGPVA |
| | | | | PGQMRDPRGSDIAGTTSNLQEQIGWMTSNPP |
| | | | | IPVGDIYKRWIIMGLNKIVRMYSPVSILDIKQ |
| | | | | GPKEPFRDYVDRFYRTLRAEQASQDVKNWM |
| | | | | TETLLVQNSNPDCKTILKALGPGATLEEMMS |
| | | | | ACQGVGGPSHKARVL |
| 90 | Gag | 142 | 166 | MVHQAISPRTLNAWVKVVEEKAFSP |
| 91 | Gag | 142 | 166 | MVHQPISPRTLNAWVKVIEEKAFSP |
| 92 | Gag | 147 | 217 | ISPRTLNAWVKVVEEKAFSPEVIPMFSALSEG |
| | | | | ATPQDLNTMLNTVGGHQAAMQMLKETINEE |
| | | | | AAEWDRLHP |
| 93 | Gag | 147 | 217 | LSPRTLNAWVKVIEEKAFSPEVIPMFTALSEG |
| | | | | ATPHDLNTMLNTIGGHQAAMQMLKDTINEE |
| | | | | AAEWDRVHP |
| 94 | Gag | 147 | 369 | ISPRTLNAWVKVVEEKAFSPEVIPMFSALSE |
| | | | | GATPQDLNTMLNTVGGHQAAMQMLKETINEE |
| | | | | AAEWDRLHPVHAGPIAPGQMREPRGSDIAGT |
| | | | | TSTLQEQIGWMTNNPPIPVGEIYKRWIILGLN |
| | | | | KIVRMYSPTSILDIRQGPKEPFRDYVDRFYKT |
| | | | | LRAEQASQEVKNWMTETLLVQNANPDCKTI |
| | | | | LKALGPAATLEEMMTACQGVGGPGHKARV |
| | | | | LAEAMSQ |
| 95 | Gag | 147 | 369 | LSPRTLNAWVKVIEEKAFSPEVIPMFTALSE |
| | | | | GATPHDLNTMLNTIGGHQAAMQMLKDTINEE |
| | | | | AAEWDRVHPVHAGPVAPGQMRDPRGSDIAG |
| | | | | STSTLQEQIAWMTNNPPIPVGDIYKRWIIMGL |
| | | | | NKIVRMYSPVSILDIKQGPKEPFRDYVDRFYR |
| | | | | TLRAEQASQDVKNWMTETLLVQNSNPDCKT |
| | | | | ILKALGPGATLEEMMSACQGVGGPSHKARV |
| | | | | LAEAMCQ |
| 96 | Gag | 150 | 158 | RTLNAWVKV |
| 97 | Gag | 175 | 199 | LSEGATPQDLNTMLNTVGGHQAAMQ |
| 98 | Gag | 175 | 199 | LSEGATPHDLNTMLNTIGGHQAAMQ |
| 99 | Gag | 183 | 191 | DLNTMLNTV |
| 100 | Gag | 183 | 191 | DLNTMLNTI |
| 101 | Gag | 225 | 251 | PGQMREPRGSDIAGTTSTLQEQIGWMT |
| 102 | Gag | 225 | 251 | PGQMRDPRGSDIAGSTSTLQEQIAWMT |
| 103 | Gag | 253 | 285 | NPPIPVGEIYKRWIILGLNKIVRMYSPTSILDI |
| 104 | Gag | 253 | 285 | NPPIPVGDIYKRWIIMGLNKIVRMYSPVSILDI |
| 339 | Gag | 257 | 282 | PVGEIYKRWIILGLNKIVRMYSPTSI |
| 340 | Gag | 257 | 282 | PVGDIYKRWIIMGLNKIVRMYSPVSI |
| 105 | Gag | 257 | 290 | PVGEIYKRWIILGLNKIVRMYSPTSILDIRQGP |
| | | | | K |
| 106 | Gag | 257 | 290 | PVGDIYKRWIIMGLNKIVRMYSPVSILDIKQG |
| | | | | PK |
| 107 | Gag | 265 | 282 | WIILGLNKIVRMYSPTSI |
| 108 | Gag | 265 | 282 | WIIMGLNKIVRMYSPVSI |
| 109 | Gag | 281 | 314 | SILDIRQGPKEPFRDYVDRFYKTLRAEQASQE |
| | | | | VK |
| 110 | Gag | 281 | 314 | SILDIKQGPKEPFRDYVDRFYRTLRAEQASQD |
| | | | | VK |
| 341 | Gag | 288 | 313 | GPKEPFRDYVDRFYKTLRAEQASQEV |
| 342 | Gag | 288 | 313 | GPKEPFRDYVDRFYRTLRAEQASQDV |
| 111 | Gag | 288 | 321 | GPKEPFRDYVDRFYKTLRAEQASQEVKNWM |
| | | | | TETL |
| 112 | Gag | 288 | 321 | GPKEPFRDYVDRFYRTLRAEQASQDVKNWM |
| | | | | TETL |
| 113 | Gag | 296 | 313 | YVDRFYKTLRAEQASQEV |
| 114 | Gag | 296 | 313 | YVDRFYRTLRAEQASQDV |
| 115 | Gag | 311 | 369 | QEVKNWMTETLLVQNANPDCKTILKALGPA |
| | | | | ATLEEMMTACQGVGGPGHKARVLAEAMSQ |
| 116 | Gag | 311 | 369 | QDVKNWMTETLLVQNSNPDCKTILKALGPG |
| | | | | ATLEEMMSACQGVGGPSHKARVLAEAMCQ |
| 117 | Gag | 333 | 357 | ILKALGPAATLEEMMTACQGVGGPG |
| 118 | Gag | 333 | 357 | ILKALGPGATLEEMMSACQGVGGPS |
| 119 | Gag | 337 | 361 | LGPAATLEEMMTACQGVGGPGHKAR |
| 120 | Gag | 337 | 361 | LGPGATLEEMMSACQGVGGPSHKAR |
| 121 | Gag | 341 | 349 | ATLEEMMTA |
| 122 | Gag | 341 | 349 | ATLEEMMSA |
| 123 | Gag | 345 | 353 | EMMTACQGV |
| 124 | Gag | 345 | 353 | EMMSACQGV |
| 125 | Gag | 391 | 400 | KCFNCGKEGH |
| 126 | Gag | 391 | 400 | KCFNCGREGH |
| 127 | Gag | 402 | 410 | ARNCRAPRK |
| 128 | Gag | 402 | 410 | AKNCRAPRK |
| 129 | Gag | 402 | 440 | ARNCRAPRKKGCWKCGKEGHQMKDCTERQ |
| | | | | ANFLGKIWPS |
| 130 | Gag | 402 | 440 | AKNCRAPRKRGCWKCGREGHQMKDCNERQ |
| | | | | ANFLGKVWPS |
| 131 | Gag | 404 | 417 | NCRAPRKKGCWKCG |
| 132 | Gag | 404 | 417 | NCRAPRKRGCWKCG |
| 133 | Gag | 412 | 430 | GCWKCGKEGHQMKDCTERQ |
| 134 | Gag | 412 | 430 | GCWKCGREGHQMKDCNERQ |
| 135 | Gag | 424 | 440 | KDCTERQANFLGKIWPS |
| 136 | Gag | 424 | 440 | KDCNERQANFLGKVWPS |
| 137 | Gag | 429 | 444 | RQANFLGKIWPSHKGR |
| 138 | Gag | 429 | 444 | RQANFLGKVWPSHNGR |
| 139 | Gag | 442 | 453 | KGRPGNFLQSRP |
| 140 | Gag | 442 | 453 | NGRPGNFLQNRP |
| 141 | Gag | 488 | 497 | SLRSLFGNDP |
| 142 | Gag | 488 | 497 | SLKSLFGNDP |
| 143 | Gag | 491 | 499 | SLFGNDPSS |
| 144 | Gag | 491 | 499 | SLFGNDPLS |
| 145 | Gag | | | LKHIVWASRELERFAVNPGLLETVSQNYPIV |
| | | | | QN |
| 146 | Gag | | | LKHLVWASRELERFALNPGLLETVSQNFPIV |
| | | | | QN |
| 147 | Nef | 29 | 37 | GVGAVSRDL |
| 148 | Nef | 29 | 37 | GVGAASRDL |
| 149 | Nef | 64 | 82 | EEVGFPVRPQVPLRPMTYK |
| 150 | Nef | 64 | 82 | EEVGFPVKPQVPLRPMTFK |
| 151 | Nef | 64 | 99 | EEVGFPVKPQVPLRPMTFKGALDLSHFLREK |
| | | | | GGLEG |
| 152 | Nef | 64 | 99 | EEVGFPVRPQVPLRPMTYKGALDLSHFLKEK |
| | | | | GGLEG |
| 153 | Ne | 81 | 102 | YKAAVDLSHFLREKGGLEGAAY |
| 154 | Nef | 81 | 102 | YKGALDLSHFLKEKGGLEGAAY |
| 155 | Nef | 88 | 97 | SHFLKEKGGL |
| 156 | Nef | 88 | 97 | SHFLREKGGL |
| 157 | Nef | 91 | 99 | LKEKGGLEG |
| 158 | Nef | 91 | 99 | LREKGGLEG |
| 159 | Nef | 117 | 132 | TQGYFPDWQNYTPGPG |
| 160 | Nef | 117 | 132 | TQGFFPDWQNYTPEPG |
| 161 | Nef | 117 | 148 | TQGFFPDWQNYTPEPGIRFPLTFGWCFKLVP |
| | | | | L |
| 162 | Nef | 117 | 148 | TQGYFPDWQNYTPGPGTRYPLTFGWCFKLV |
| | | | | PV |
| 163 | Nef | 130 | 148 | EPGIRFPLTFGWCFKLVPL |
| 164 | Nef | 130 | 148 | GPGTRYPLTFGWCFKLVPV |
| 165 | Nef | 130 | 154 | GPGIRYPLLTFGWCFKLPVEPEKVE |
| 166 | Nef | 134 | 142 | RYPLTFGWC |
| 167 | Nef | 134 | 142 | RFPLTFGWC |
| 168 | Nef | 134 | 148 | RYPLTFGWCFKLVPV |
| 169 | Nef | 134 | 148 | RFPLTFGWCFKLVPL |
| 170 | Nef | 136 | 148 | PLTFGWCFKLVPV |
| 171 | Nef | 136 | 148 | PLCFGWCFKLVPL |
| 172 | Nef | 137 | 145 | LTFGWCFKL |
| 173 | Nef | 137 | 145 | LCFGWCFKL |
| 174 | Pol | 56 | 67 | FPQITLWQRPLV |
| 175 | Pol | 56 | 67 | LPQITLWQRPIV |
| 176 | Pol | 56 | 117 | FPQITLWQRPLVTIKIGGQLKEALLDTGADDT |
| | | | | VLEEMNLPGRWKPKMIGGIGGFIKVRQYDQ |
| 177 | Pol | 56 | 117 | LPQITLWQRPIVTIKIGGQIKEALLDTGADDT |
| | | | | VLEDMNLPGKWKPKMIGGIGGFIKVKQYDQ |
| 178 | Pol | 72 | 91 | GGQLKEALLDTGADDTVLEE |
| 179 | Pol | 72 | 91 | GGQIKEALLDTGADDTVLED |
| 180 | Pol | 94 | 117 | LPGRWKPKMIGGIGGFIKVRQYDQ |
| 181 | Pol | 94 | 117 | LPGKWKPKMIGGIGGFIKVKQYDQ |
| 182 | Pol | 129 | 260 | GTVLVGPTPVNIIGRNLLTQIGCTLNFPISPIET |
| | | | | VPVKLKPGMDGPKVKQWPLTEEKIKALVEIC |
| | | | | TEMEKEGKISKIGPENPYNTPVFAIKKKDSTK |
| | | | | WRKLVDFRELNKRTQDFWEVQLGIPHPAGL |
| | | | | KKKKS |
| 183 | Pol | 129 | 260 | GTVLIGPTPVNIIGRNLLTQLGCTLNFPISPIDT |
| | | | | VPVKLKPGMDGPRVKQWPLTEEKIKALIEIC |
| | | | | TEMEKEGKISRIGPENPYNTPIFAIKKKDGTK |
| | | | | WRKLVDFRELNKKTQDFWEVQLGIPHPSGL |
| | | | | KKKKS |
| 184 | Pol | 129 | 277 | GTVLVGPTPVNIIGRNLLTQIGCTLNFPISPIET |
| | | | | VPVKLKPGMDGPKVKQWPLTEEKIKALVEIC |
| | | | | TEMEKEGKISKIGPENPYNTPVFAIKKKDSTK |
| | | | | WRKLVDFRELNKRTQDFWEVQLGIPHPAGL |
| | | | | KKKKSVTVLDVGDAYFSVPLDK |
| 185 | Pol | 129 | 277 | GTVLIGPTPVNIIGRNLLTQLGCTLNFPISPIDT |
| | | | | VPVKLKPGMDGPRVKQWPLTEEKIKALIEIC |
| | | | | TEMEKEGKISRIGPENPYNTPIFAIKKKDGTK |
| | | | | WRKLVDFRELNKKTQDFWEVQLGIPHPSGL |
| | | | | KKKKSVTILDVGDAYFSIPLDK |
| 186 | Pol | 129 | 289 | GTVLVGPTPVNIIGRNLLTQIGCTLNFPISPIET |
| | | | | VPVKLKPGMDGPKVKQWPLTEEKIKALVEIC |
| | | | | TEMEKEGKISKIGPENPYNTPVFAIKKKDSTK |
| | | | | WRKLVDFRELNKRTQDFWEVOLGIPHPAGL |
| | | | | KKKKSVTVLDVGDAYFSVPLDKDFRKYTAF |
| | | | | TIPS |
| 187 | Pol | 129 | 289 | GTVLIGPTPVNIIGRNLLTQLGCTLNFPISPIDT |
| | | | | VPVKLKPGMDGPRVKQWPLTEEKIKALIEIC |
| | | | | TEMEKEGKISRIGPENPYNTPIFAIKKKDGTK |
| | | | | WRKLVDFRELNKKTQDFWEVQLGIPHPSGL |
| | | | | KKKKSVTVLDIGDAYFSVPLDKEFRKYTAFT |
| | | | | VPS |
| 188 | Pol | 129 | 320 | GTVLVGPTPVNIIGRNLLTQIGCTLNFPISPIET |
| | | | | VPVKLKPGMDGPKVKQWPLTEEKIKALVEIC |
| | | | | TEMEKEGKISKIGPENPYNTPVFAIKKKDSTK |
| | | | | WRKLVDFRELNKRTQDFWEVQLGIPHPAGL |
| | | | | KKKKSVTVLDVGDAYFSVPLDKDFRKYTAF |
| | | | | TIPSINNETPGIR YQYNVLPQGWKGSPAIFQSS |
| | | | | MT |
| 189 | Pol | 129 | 320 | GTVLIGPTPVNIIGRNLLTQLGCTLNFPISPIDT |
| | | | | VPVKLKPGMDGPRVKQWPLTEEKIKALIEIC |
| | | | | TEMEKEGKISRIGPENPYNTPIFAIKKKDGTK |
| | | | | WRKLVDFRELNKKTQDFWEVQLGIPHPSGL |
| | | | | KKKKSVTVLDIGDAYFSVPLDKEFRKYTAFT |
| | | | | VPSTNNETPGVRYQYNVLPMGWKGSPAIFQ |
| | | | | CSMT |
| 190 | Pol | 144 | 168 | NLLTQIGCTLNFPISPIETVPVKLK |
| 191 | Pol | 144 | 168 | NLLTQLGCTLNFPISPIDTVPVKLK |
| 192 | Pol | 152 | 160 | TLNFPISPI |
| 193 | Pol | 254 | 277 | GLKKKKSVTVLDVGDAYFSVPLDK |
| 194 | Pol | 254 | 277 | GLKKNKSVTVLDVGDAYFSIPLDK |
| 195 | Pol | 278 | 289 | DFRKYTAFTIPS |
| 196 | Pol | 278 | 289 | EFRKYTAFTVPS |
| 197 | Pol | 291 | 315 | NNETPGIRYQYNVLPQGWKGSPAIF |
| 198 | Pol | 291 | 315 | NNETPGVRYQYNVLPMGWKGSPAIF |
| 199 | Pol | 291 | 320 | NNETPGIRYQYNVLPQGWKGSPAIFQSSMT |
| 200 | Pol | 291 | 320 | NNETPGVRYQYNVLPMGWKGSPAIFQCSMT |
| 201 | Pol | 299 | 307 | YQYNVLPQG |
| 202 | Pol | 299 | 307 | YQYNVLPMG |
| 203 | Pol | 315 | 323 | FQSSMTKIL |
| 204 | Pol | 315 | 323 | FQCSMTKIL |
| 205 | Pol | 318 | 327 | SMTKILEPFR |
| 206 | Pol | 318 | 327 | SMTKILDPFR |
| 207 | Pol | 322 | 330 | ILEPFRKQN |
| 208 | Pol | 322 | 330 | ILDPFRKQN |
| 209 | Pol | 326 | 350 | FRKQNPDIVIYQYMDDLYVGSDLEI |
| 343 | Pol | 326 | 350 | FRKQNPDIVIYQYVDDLYVGSDLEI |
| 210 | Pol | 328 | 352 | KQNPDIVIYQYMDDLYVGSDLEIGQ |
| 344 | Pol | 328 | 352 | KQNPDIVIYQYVDDLYVGSDLEIEQ |
| 211 | Pol | 330 | 354 | NPDIVIYQYMDDLYVGSDLEIGQHR |
| 212 | Pol | 330 | 354 | NPDIVIYQYVDDLYVGSDLEIEQHR |
| 213 | Pol | 333 | 354 | IVIYQYMDDLYVGSDLEIGQHR |
| 214 | Pol | 333 | 354 | IVIYQYVDDLYVGSDLEIEQHR |
| 215 | Pol | 334 | 342 | VIYQYMDDL |
| 216 | Pol | 334 | 342 | VIYQYVDDL |
| 217 | Pol | 336 | 344 | YQYMDDLYV |
| 218 | Pol | 336 | 344 | YQYVDDLYV |
| 219 | Pol | 338 | 346 | YMDDLYVGS |
| 220 | Pol | 338 | 346 | YVDDLYVGS |
| 221 | Pol | 367 | 399 | WGFTTPDKKHQKEPPFLWMGYELHPDKWT |
| | | | | VQPI |
| 222 | Pol | 367 | 399 | WGLTTPDKKHQKDPPFLWMGYELHPDRWT |
| | | | | VQPI |
| 223 | Pol | 367 | 431 | WGFTTPDKKHQKEPPFLWMGYELHPDKWTVQ |
| | | | | PIVLPEKDSWTVNDIQKLVGKLNWASQIY |
| | | | | PGIKV |
| 224 | Pol | 367 | 431 | WGLTTPDKKHQKDPPFLWMGYELHPDRWTVQ |
| | | | | PIELPEKESWTVNDIQKLIGKLNWASQIYA |
| | | | | GIKV |
| 225 | Pol | 374 | 398 | KKHQKEPPFLWMGYELHPDKWTVQP |
| 226 | Pol | 374 | 398 | KKHQKDPPFLWMGYELHPDRWTVQP |
| 227 | Pol | 380 | 404 | PPFLWMGYELHPDKWTVQPIVLPEK |
| 228 | Pol | 380 | 404 | PPFLWMGYELHPDRWTVQPIELPEK |
| 229 | Pol | 382 | 390 | FLWMGYELH |
| 230 | Pol | 388 | 396 | ELHPDKWTV |
| 231 | Pol | 388 | 396 | ELHPDRWTV |
| 232 | Pol | 399 | 423 | IVLPEKDSWTVNDIQKLVGKLNWAS |
| 233 | Pol | 399 | 423 | IELPEKESWTVNDIQKLIGKLNWAS |
| 234 | Pol | 400 | 424 | VLPEKDSWTVNDIQKLVGKLNWASQ |
| 235 | Pol | 400 | 424 | ELPEKESWTVNDIQKLIGKLNWASQ |
| 236 | Pol | 401 | 431 | LPEKDSWTVNDIQKLVGKLNWASQIYPGIKV |
| 237 | Pol | 401 | 431 | LPEKESWTVNDIQKLIGKLNWASQIYAGIKV |
| 238 | Pol | 406 | 430 | SWTVNDIQKLVGKLNWASQIYPGIK |
| 239 | Pol | 406 | 430 | SWTVNDIQKLIGKLNWASQIYAGIK |
| 240 | Pol | 407 | 415 | WTVNDIQKL |
| 241 | Pol | 408 | 416 | TVNDIQKLV |
| 242 | Pol | 408 | 416 | TVNDIQKLI |
| 243 | Pol | 414 | 422 | KLVGKLNWA |
| 244 | Pol | 414 | 422 | KLIGKLNWA |
| 245 | Pol | 434 | 442 | LCKLLRGTK |
| 246 | Pol | 434 | 442 | LCKLLRGAK |
| 247 | Pol | 453 | 471 | EAELELAENREILKEPVHG |
| 248 | Pol | 453 | 471 | EAEIELAENREILREPVHG |
| 249 | Pol | 467 | 478 | EPVHGVYYDPSK |
| 250 | Pol | 467 | 478 | EPVHGAYYDPSK |
| 251 | Pol | 490 | 511 | GQWTYQIYQEPFKNLKTGKYAR |
| 252 | Pol | 490 | 511 | GQWSYQIYQEPYKNLKTGKYAK |
| 253 | Pol | 515 | 530 | AHTNDVKQLTEAVQKI |
| 254 | Pol | 515 | 530 | AHTNDVRQLTEAVQKV |
| 255 | Pol | 535 | 544 | IVIWGKTPKF |
| 256 | Pol | 535 | 544 | IVIWGKIPKF |
| 257 | Pol | 542 | 554 | PKFKLPIQKETWE |
| 258 | Pol | 542 | 554 | PKFRLPIQKETWD |
| 259 | Pol | 542 | 606 | PKFKLPIQKETWETWWTEYWQATWIPEWEF |
| | | | | VNTPPLVKLWYQLEKEPIVGAETFYVDGAA |
| | | | | NRETK |
| 260 | Pol | 542 | 606 | PKFRLPIQKETWDTWWTDYWQATWIPEWEF |
| | | | | TNTPPLVKLWYQLETEPIAGVETFYVDGASN |
| | | | | RETK |
| 261 | Pol | 553 | 577 | WETWWTEYWQATWIPEWEFVNTPPL |
| 262 | Pol | 553 | 577 | WDTWWTDYWQATWIPEWEFTNTPPL |
| 263 | Pol | 559 | 589 | EYWQATWIPEWEFVNTPPLVKLWYQLEKEPI |
| 264 | Pol | 559 | 589 | DYWQATWIPEWEFTNTPPLVKLWYQLETEPI |
| 265 | Pol | 561 | 569 | WQATWIPEW |
| 266 | Pol | 591 | 606 | GAETFYVDGAANRETK |
| 267 | Pol | 591 | 606 | GVETFYVDGASNRETK |
| 268 | Pol | 625 | 637 | TDTTNQKTELQAI |
| 269 | Pol | 625 | 637 | ADTTNQKTELHAI |
| 270 | Pol | 636 | 644 | AIHLALQDS |
| 271 | Pol | 636 | 644 | AIYLALQDS |
| 272 | Pol | 639 | 671 | LALQDSGLEVNIVTDSQYALGIIQAQPDKSES |
| | | | | E |
| 273 | Pol | 639 | 671 | LALQDSGSEVNIVTDSQYAIGIIQAQPDRSESE |
| 274 | Pol | 642 | 666 | QDSGLEVNIVTDSQYALGIIQAQPD |
| 275 | Pol | 642 | 666 | QDSGSEVNIVTDSQYAIGIIQAQPD |
| 276 | Pol | 650 | 658 | IVTDSQYAL |
| 277 | Pol | 650 | 658 | IVTDSQYAI |
| 278 | Pol | 673 | 681 | VSQIIEQLI |
| 279 | Pol | 673 | 681 | VNQIIEQLI |
| 280 | Pol | 675 | 684 | QIIEQLIKKE |
| 281 | Pol | 675 | 684 | QIIEQLINKE |
| 282 | Pol | 683 | 708 | KEKVYLAWVPAHKGIGGNEQVDKLVS |
| 283 | Pol | 683 | 708 | KEKIYLAWVPAHKGIGGNEQIDKLVS |
| 284 | Pol | 710 | 725 | GIRKVLFLDGIDKAQE |
| 285 | Pol | 710 | 725 | GIRRVLFLDGIEKAQD |
| 286 | Pol | 727 | 735 | HEKYHSNWR |
| 287 | Pol | 727 | 735 | HEKYHNNWR |
| 288 | Pol | 737 | 745 | MASDFNLPP |
| 289 | Pol | 737 | 745 | MASDFNIPP |
| 290 | Pol | 741 | 753 | FNLPPVVAKEIVA |
| 291 | Pol | 741 | 753 | FNLPPIVAKEIVA |
| 292 | Pol | 741 | 827 | FNLPPVVAKEIVASCDKCQLKGEAMHGQVD |
| | | | | CSPGIWQLDCTHLEGKIILVAVHVASGYIEAE |
| | | | | VIPAETGQETAYFLLKLAGRWPVKT |
| 293 | Pol | 741 | 827 | FNLPPIVAKEIVACCDKCQLKGEAIHGQVDC |
| | | | | SPGVWQLDCTHLEGKVILVAVHVASGYIEAE |
| | | | | IIPTETGQETAYFILKLAGRWPVTT |
| 294 | Pol | 747 | 827 | VAKEIVASCDKCQLKGEAMHGQVDCSPGIW |
| | | | | QLDCTHLEGKIILVAVHVASGYIEAEVIPAET |
| | | | | GQETAYFLLKLAGRWPVKT |
| 295 | Pol | 747 | 827 | VAKEIVACCDKCQLKGEAIHGQVDCSPGVW |
| | | | | QLDCTHLEGKVILVAVHVASGYMEAEVIPTE |
| | | | | TGQETAYFILKLAGRWPVTT |
| 296 | Pol | 759 | 783 | QLKGEAMHGQVDCSPGIWQLDCTHL |
| 297 | Pol | 759 | 783 | QLKGEAIHGQVDCSPGVWQLDCTHL |
| 298 | Pol | 767 | 775 | GQVDCSPGI |
| 299 | Pol | 767 | 775 | GQVDCSPGV |
| 300 | Pol | 768 | 792 | QVDCSPGIWQLDCTHLEGKIILVAV |
| 301 | Pol | 768 | 792 | QVDCSPGVWQLDCTHLEGKVILVAV |
| 302 | Pol | 776 | 784 | WQLDCTHLE |
| 303 | Pol | 834 | 858 | SNFTSTTVKAACWWAGIKQEFGIPY |
| 304 | Pol | 834 | 858 | SNFTSTAVKAACWWAGVKQEFGIPY |
| 305 | Pol | 840 | 919 | TVKAACWWAGIKQEFGIPYNPQSQGVVESM |
| | | | | NKELKKIIGQVRDQAEHLKTAVQMAVFIHNF |
| | | | | KRKGGIGGYSAGERIVDII |
| 306 | Pol | 840 | 919 | AVKAACWWAGVKQEFGIPYHPQSQGVVES |
| | | | | MNNELKKIIGQIRDQAEQLKTAVQMAVLIHN |
| | | | | FKRKGGIGEYSAGERIIDII |
| 307 | Pol | 840 | 920 | TVKAACWWAGIKQEFGIPYNPQSQGVVESM |
| | | | | NKELKKIIGQVRDQAEHLKTAVQMAVFIHNF |
| | | | | KRKGGIGGYSAGERIVDIIA |
| 308 | Pol | 840 | 920 | AVKAACWWAGVKQEFGIPYHPQSQGVVES |
| | | | | MNNELKKIIGQIRDQAEQLKTAVQMAVLIHN |
| | | | | FKRKGGIGEYSAGERIIDIIA |
| 309 | Pol | 840 | 1003 | TVKAACWWAGIKQEFGIPYNPQSQGVVESM |
| | | | | NKELKKIIGQVRDQAEHLKTAVQMAVFIHNF |
| | | | | KRKGGIGGYSAGERIVDIIATDIQTKELQKQIT |
| | | | | KIQNFRVYYRDSRDPLWKGPAKLLWKGEGA |
| | | | | VVIQDNSDIKVVPRRKAKIIRDYGKQMAGDD |
| | | | | CVASRQDED |
| 310 | Pol | 840 | 1003 | AVKAACWWAGVKQEFGIPYNTQSQGVVES |
| | | | | MNNELKKIIGQIRDQAEHLKTAVQMAVLIHN |
| | | | | FKRKGGIGEYSAGERIIDIIATDIQTRELQKQIT |
| | | | | KLQNFRVYYRDNRDPLWKGPARLLWKGEG |
| | | | | AVVIQDNSEIKVVPRRKVKIIRDYGKRMAGD |
| | | | | DCVAGRQDED |
| 311 | Pol | 842 | 850 | KAACWWAGI |
| 312 | Pol | 842 | 850 | KAACWWAGV |
| 313 | Pol | 917 | 925 | DIIATDIQT |
| 314 | Pol | 917 | 925 | DIIASDIQT |
| 315 | Pol | 922 | 930 | DIQTKELQK |
| 316 | Pol | 922 | 930 | DIQTRELQK |
| 317 | Pol | 924 | 932 | QTKELQKQI |
| 318 | Pol | 924 | 932 | QTRELQKQI |
| 319 | Pol | 931 | 1003 | AITKIQNFRVYYRDSRDPLWKGPAKLLWKG |
| | | | | EGAVVIQDNSDIKVVPRRKAKIIRDYGKQMA |
| | | | | GDDCVASRQDED |
| 320 | Pol | 931 | 1003 | AITKLQNFRVYYRDNRDPLWKGPARLLWKG |
| | | | | EGAVVIQDNSEIKVVPRRKVKIIRDYGKRMA |
| | | | | GDDCVAGRQDED |
| 321 | Pol | 932 | 1003 | ITKIQNFRVYYRDSRDPLWKGPAKLLWKGE |
| | | | | GAVVIQDNSDIKVVPRRKAKIIRDYGKQMAG |
| | | | | DDCVASRQDED |
| 322 | Pol | 932 | 1003 | ITKLQNFRVYYRDNRDPLWKGPARLLWKGE |
| | | | | GAVVIQDNSEIKVVPRRKVKIIRDYGKRMAG |
| | | | | DDCVAGRQDED |
| 323 | Pol | 940 | 964 | VYYRDSRDPLWKGPAKLLWKGEGAV |
| 324 | Pol | 940 | 964 | VYYRDNRDPLWKGPARLLWKGEGAV |
| 325 | Pol | 947 | 971 | DPLWKGPAKLLWKGEGAVVIQDNSD |
| 326 | Pol | 947 | 971 | DPLWKGPARLLWKGEGAVVIQDNSE |
| 327 | Pol | 948 | 956 | PLWKGPAKL |
| 328 | Pol | 948 | 956 | PLWKGPARL |
| 329 | Pol | 948 | 972 | PLWKGPAKLLWKGEGAVVIQDNSDI |
| 330 | Pol | 948 | 972 | PLWKGPARLLWKGEGAVVIQDNSEI |
| 331 | Pol | 955 | 963 | KLLWKGEGA |
| 332 | Pol | 955 | 963 | RLLWKGEGA |
| 333 | Pol | 956 | 964 | LLWKGEGAV |
| 334 | Pol | 980 | 1003 | AKIIRDYGKQMAGDDCVASRQDED |
| 335 | Pol | 980 | 1003 | VKIIRDYGKRMAGDDCVAGRQDED |
| 336 | Pol | 988 | 996 | KQMAGDDCV |
| 337 | Pol | 988 | 996 | KRMAGDDCV |

TABLE 3
polypeptide segments in conserved regions of HIV-1 proteins

| SEQ ID NO: | Gene | Start | End | Length | Sequence |
|---|---|---|---|---|---|
| 2 | Env | 34 | 48 | 15 | LWVTVYYGVPVWKEA |
| 3 | Env | 34 | 48 | 15 | LWVTIYYGVPVWKDA |
| 8 | Env | 48 | 61 | 14 | ATTTLFCASDAKAY |
| 9 | Env | 48 | 61 | 14 | ANTTLFCASDAKGY |
| 13 | Env | 66 | 83 | 18 | HNVWATHACVPTDPNPQE |
| 14 | Env | 66 | 83 | 18 | HNIWATHACVPTDPSPQE |
| 17 | Env | 107 | 129 | 23 | DIISLWDQSLKPCVKLTPLCVTL |
| 18 | Env | 107 | 129 | 23 | DIISLWDESLKPCVKLTPICVTL |
| 23 | Env | 209 | 226 | 18 | SFEPIPIHYCAPAGFAIL |
| 24 | Env | 209 | 226 | 18 | TFEPIPIHYCTPAGFAIL |
| 25 | Env | 220 | 228 | 9 | PAGFAILKC |
| 26 | Env | 220 | 228 | 9 | PAGFALLKC |
| 28 | Env | 241 | 268 | 28 | NVSTVQCTHGIRPVVSTQLLLNGSLAEE |
| 29 | Env | 241 | 268 | 28 | NISTVQCTHGIKPVVSTQLLLNGSLAEK |
| 31 | Env | 376 | 386 | 11 | FNCGGEFFYCN |
| 32 | Env | 376 | 386 | 11 | FNCRGEFFYCN |
| 33 | Env | 430 | 439 | 10 | VGKAMYAPPI |
| 34 | Env | 430 | 439 | 10 | VGRAMYAPPI |
| 35 | Env | 472 | 481 | 10 | GGDMRDNWRS |
| 36 | Env | 472 | 481 | 10 | GGNMKDNWRS |
| 37 | Env | 475 | 489 | 15 | MRDNWRSELYKYKVV |
| 38 | Env | 475 | 489 | 15 | MKDNWRSELYRYKVV |
| 39 | Env | 501 | 511 | 11 | AKRRVVQREKR |
| 40 | Env | 501 | 511 | 11 | ARRRVVQREKR |
| 43 | Env | 519 | 534 | 16 | FLGFLGAAGSTMGAAS |
| 44 | Env | 519 | 534 | 16 | FLGFLGTAGSTMGAAA |
| 45 | Env | 533 | 606 | 74 | ASITLTVQARQLLSGIVQQQNNLLRAIEA |
| | | | | | QQHLLQLTVWGIKQLQARVLAVERYLK |
| | | | | | DQQLLGIWGCSGKLICTT |
| 46 | Env | 533 | 606 | 74 | ASMTLTVQARLLLSGIVQQQSNLLRAIE |
| | | | | | AQQHMLQLTVWGIKQLQARILAVERYL |
| | | | | | RDQQLLGIWGCSGRLICTT |
| 47 | Env | 536 | 556 | 21 | TLTVQARQLLSGIVQQQNNLL |
| 48 | Env | 536 | 556 | 21 | TLTVQARLLLSGIVQQQSNLL |
| 49 | Env | 554 | 564 | 11 | NLLRAIEAQQH |
| 50 | Env | 554 | 564 | 11 | NLLKAIEAQQH |
| 51 | Env | 558 | 584 | 27 | AIEAQQHLLQLTVWGIKQLQARVLAVE |
| 52 | Env | 558 | 584 | 27 | AIEAQQHMLQLTVWGIKQLQARILAVE |
| 53 | Env | 584 | 592 | 9 | ERYLKDQQL |
| 54 | Env | 584 | 592 | 9 | ERYLRDQQL |
| 55 | Env | 586 | 594 | 9 | YLKDQQLLG |
| 56 | Env | 586 | 594 | 9 | YLRDQQLLG |
| 58 | Env | 589 | 606 | 18 | DQQLLGIWGCSGKLICTT |
| 59 | Env | 589 | 606 | 18 | DQQLLGLWGCSGKLICPT |
| 62 | Env | 678 | 688 | 11 | WLWYIKIFIMI |
| 63 | Env | 678 | 688 | 11 | WLWYIRIFIMI |
| 64 | Env | 684 | 697 | 14 | IFIMIVGGLIGLRI |
| 65 | Env | 684 | 697 | 14 | LFIMIVGGLVGLRI |
| 66 | Env | 705 | 719 | 15 | VNRVRQGYSPLSFQT |
| 67 | Env | 705 | 719 | 15 | VNRVRKGYSPLSFQI |
| 68 | Gag | 1 | 11 | 11 | MGARASVLSGG |
| 69 | Gag | 1 | 11 | 11 | MGARASILSGG |
| 72 | Gag | 13 | 25 | 13 | LDRWEKIRLRPGG |
| 73 | Gag | 13 | 25 | 13 | LDKWEKIRLRPMG |
| 74 | Gag | 19 | 27 | 9 | IRLRPGGKK |
| 75 | Gag | 19 | 27 | 9 | IRLRPGGRK |
| 76 | Gag | 31 | 53 | 23 | LKHIVWASRELERFA VNPGLLET |
| 77 | Gag | 31 | 53 | 23 | LKHLVWASRELERFALNPGLLET |
| 80 | Gag | 70 | 78 | 9 | TGSEELKSL |
| 81 | Gag | 70 | 78 | 9 | TGSEELRSL |
| 82 | Gag | 96 | 104 | 9 | DTKEALDKI |
| 83 | Gag | 96 | 104 | 9 | DTKEALEKI |
| 84 | Gag | 99 | 107 | 9 | EALDKIEEE |
| 85 | Gag | 99 | 107 | 9 | EALEKIEEE |
| 86 | Gag | 128 | 137 | 10 | VSQNYPIVQN |
| 87 | Gag | 128 | 137 | 10 | VSQNFPIVQN |
| 88 | Gag | 133 | 363 | 231 | PIVQNLQGQMVHQAISPRTLNAWVKVV |
| | | | | | EEKAFSPEVIPMFSALSEGATPQDLNTML |
| | | | | | NTVGGHQAAMQMLKETINEEAAEWDR |
| | | | | | LHPVHAGPIAPGQMREPRGSDIAGTTSTL |
| | | | | | QEQIGWMTNNPPIPVGEIYKRWIILGLNK |
| | | | | | IVRMYSPTSILDIRQGPKEPFRDYVDRFY |
| | | | | | KTLRAEQASQEVKNWMTETLLVQNANP |
| | | | | | DCKTILKALGPAATLEEMMTACQGVGG |
| | | | | | PGHKARVL |
| 89 | Gag | 133 | 363 | 231 | PIVQNIQGQMVHQPISPRTLNAWVKVIEE |
| | | | | | KAFSPEVIPMFTALSEGATPHDLNTMLN |
| | | | | | TIGGHQAAMQMLKDTINEEAAEWDRVH |
| | | | | | PVHAGPVAPGQMRDPRGSDIAGTTSNLQ |
| | | | | | EQIGWMTSNPPIPVGDIYKRWIIMGLNKI |
| | | | | | VRMYSPVSILDIKQGPKEPFRDYVDRFY |
| | | | | | RTLRAEQASQDVKNWMTETLLVQNSNP |
| | | | | | DCKTILKALGPGATLEEMMSACQGVGG |
| | | | | | PSHKARVL |
| 92 | Gag | 147 | 217 | 71 | ISPRTLNAWVKVVEEKAFSPEVIPMFSAL |
| | | | | | SEGATPQDLNTMLNTVGGHQAAMQML |
| | | | | | KETINEEAAEWDRLHP |
| 93 | Gag | 147 | 217 | 71 | LSPRTLNAWVKVIEEKAFSPEVIPMFTAL |
| | | | | | SEGATPHDLNTMLNTIGGHQAAMQMLK |
| | | | | | DTINEEAAEWDRVHP |
| 101 | Gag | 225 | 251 | 27 | PGQMREPRGSDIAGTTSTLQEQIGWMT |
| 102 | Gag | 225 | 251 | 27 | PGQMRDPRGSDIAGSTSTLQEQIAWMT |
| 103 | Gag | 253 | 285 | 33 | NPPIPVGEIYKRWIILGLNKIVRMYSPTSI |
| | | | | | LDI |
| 104 | Gag | 253 | 285 | 33 | NPPIPVGDIYKRWIIMGLNKIVRMYSPVS |
| | | | | | ILDI |
| 109 | Gag | 281 | 314 | 34 | SILDIRQGPKEPFRDYVDRFYKTLRAEQA |
| | | | | | SQEVK |
| 110 | Gag | 281 | 314 | 34 | SILDIKQGPKEPFRDYVDRFYRTLRAEQA |
| | | | | | SQDVK |
| 115 | Gag | 311 | 369 | 59 | QEVKNWMTETLLVQNANPDCKTILKAL |
| | | | | | GPAATLEEMMTACQGVGGPGHKARVL |
| | | | | | AEAMSQ |
| 116 | Gag | 311 | 369 | 59 | QDVKNWMTETLLVQNSNPDCKTILKAL |
| | | | | | GPGATLEEMMSACQGVGGPSHKARVLA |
| | | | | | EAMCQ |
| 125 | Gag | 391 | 400 | 10 | KCFNCGKEGH |
| 126 | Gag | 391 | 400 | 10 | KCFNCGREGH |
| 127 | Gag | 402 | 410 | 9 | ARNCRAPRK |
| 128 | Gag | 402 | 410 | 9 | AKNCRAPRK |
| 129 | Gag | 402 | 440 | 39 | ARNCRAPRKKGCWKCGKEGHQMKDCT |
| | | | | | ERQANFLGKIWPS |
| 130 | Gag | 402 | 440 | 39 | AKNCRAPRKRGCWKCGREGHQMKDCN |
| | | | | | ERQANFLGKVWPS |
| 131 | Gag | 404 | 417 | 14 | NCRAPRKKGCWKCG |
| 132 | Gag | 404 | 417 | 14 | NCRAPRKRGCWKCG |
| 133 | Gag | 412 | 430 | 19 | GCWKCGKEGHQMKDCTERQ |
| 134 | Gag | 412 | 430 | 19 | GCWKCGREGHQMKDCNERQ |
| 135 | Gag | 424 | 440 | 17 | KDCTERQANFLGKIWPS |
| 136 | Gag | 424 | 440 | 17 | KDCNERQANFLGKVWPS |
| 139 | Gag | 442 | 453 | 12 | KGRPGNFLQSRP |
| 140 | Gag | 442 | 453 | 12 | NGRPGNFLQNRP |
| 141 | Gag | 488 | 497 | 10 | SLRSLFGNDP |
| 142 | Gag | 488 | 497 | 10 | SLKSLFGNDP |
| 143 | Gag | 491 | 499 | 9 | SLFGNDPSS |
| 144 | Gag | 491 | 499 | 9 | SLFGNDPLS |
| 145 | Gag | | | | LKHIVWASRELERFAVNPGLLETVSQNY |
| | | | | | PIVQN |
| 146 | Gag | | | | LKHLVWASRELERFALNPGLLETVSQNF |
| | | | | | PIVQN |
| 147 | Nef | 29 | 37 | 9 | GVGAVSRDL |
| 148 | Nef | 29 | 37 | 9 | GVGAASRDL |
| 149 | Nef | 64 | 82 | 19 | EEVGFPVRPQVPLRPMTYK |
| 150 | Nef | 64 | 82 | 19 | EEVGFPVKPQVPLRPMTFK |
| 155 | Nef | 88 | 97 | 10 | SHFLKEKGGL |
| 156 | Nef | 88 | 97 | 10 | SHFLREKGGL |
| 157 | Nef | 91 | 99 | 9 | LKEKGGLEG |
| 158 | Nef | 91 | 99 | 9 | LREKGGLEG |
| 159 | Nef | 117 | 132 | 16 | TQGYFPDWQNYTPGPG |
| 160 | Nef | 117 | 132 | 16 | TQGFFPDWQNYTPEPG |
| 166 | Nef | 134 | 142 | 9 | RYPLTFGWC |
| 167 | Nef | 134 | 142 | 9 | RFPLTFGWC |
| 168 | Nef | 134 | 148 | 15 | RYPLTFGWCFKLVPV |
| 169 | Nef | 134 | 148 | 15 | RFPLTFGWCFKLVPL |
| 170 | Nef | 136 | 148 | 13 | PLTFGWCFKLVPV |
| 171 | Nef | 136 | 148 | 13 | PLCFGWCFKLVPL |
| 174 | Pol | 56 | 67 | 12 | FPQITLWQRPLV |
| 175 | Pol | 56 | 67 | 12 | LPQITLWQRPIV |
| 178 | Pol | 72 | 91 | 20 | GGQLKEALLDTGADDTVLEE |
| 179 | Pol | 72 | 91 | 20 | GGQIKEALLDTGADDTVLED |
| 180 | Pol | 94 | 117 | 24 | LPGRWKPKMIGGIGGFIKVRQYDQ |
| 181 | Pol | 94 | 117 | 24 | LPGKWKPKMIGGIGGFIKVKQYDQ |
| 182 | Pol | 129 | 260 | 132 | GTVLVGPTPVNIIGRNLLTQIGCTLNFPIS |
| | | | | | PIETVPVKLKPGMDGPKVKQWPLTEEKI |
| | | | | | KALVEICTEMEKEGKISKIGPENPYNTPV |
| | | | | | FAIKKKDSTKWRKLVDFRELNKRTQDF |
| | | | | | WEVQLGIPHPAGLKKKKS |
| 183 | Pol | 129 | 260 | 132 | GTVLIGPTPVNIIGRNLLTQLGCTLNFPIS |
| | | | | | PIDTVPVKLKPGMDGPRVKQWPLTEEKI |
| | | | | | KALIEICTEMEKEGKISRIGPENPYNTPIF |
| | | | | | AIKKKDGTKWRKLVDFRELNKKTQDFW |
| | | | | | EVQLGIPHPSGLKKKKS |
| 184 | Pol | 129 | 277 | 149 | GTVLVGPTPVNIIGRNLLTQIGCTLNFPIS |
| | | | | | PIETVPVKLKPGMDGPKVKQWPLTEEKI |
| | | | | | KALVEICTEMEKEGKISKIGPENPYNTPV |
| | | | | | FAIKKKDSTKWRKLVDFRELNKRTQDF |
| | | | | | WEVQLGIPHPAGLKKKKSVTVLDVGDA |
| | | | | | YFSVPLDK |
| 185 | Pol | 129 | 277 | 149 | GTVLIGPTPVNIIGRNLLTQLGCTLNFPIS |
| | | | | | PIDTVPVKLKPGMDGPRVKQWPLTEEKI |
| | | | | | KALIEICTEMEKEGKISRIGPENPYNTPIF |
| | | | | | AIKKKDGTKWRKLVDFRELNKKTQDFW |
| | | | | | EVQLGIPHPSGLKKKKSVTILDVGDAYFS |
| | | | | | IPLDK |
| 193 | Pol | 254 | 277 | 24 | GLKKKKSVTVLDVGDAYFSVPLDK |
| 194 | Pol | 254 | 277 | 24 | GLKKNKSVTVLDVGDAYFSIPLDK |
| 195 | Pol | 278 | 289 | 12 | DFRKYTAFTIPS |
| 196 | Pol | 278 | 289 | 12 | EFRKYTAFTVPS |
| 197 | Pol | 291 | 315 | 25 | NNETPGIRYQYNVLPQGWKGSPAIF |
| 198 | Pol | 291 | 315 | 25 | NNETPGVRYQYNVLPMGWKGSPAIF |
| 199 | Pol | 291 | 320 | 30 | NNETPGIRYQYNVLPQGWKGSPAIFQSSMT |
| 200 | Pol | 291 | 320 | 30 | NNETPGVRYQYNVLPMGWKGSPAIFQCSMT |
| 203 | Pol | 315 | 323 | 9 | FQSSMTKIL |
| 204 | Pol | 315 | 323 | 9 | FQCSMTKIL |
| 205 | Pol | 318 | 327 | 10 | SMTKILEPFR |
| 206 | Pol | 318 | 327 | 10 | SMTKILDPFR |
| 207 | Pol | 322 | 330 | 9 | ILEPFRKQN |
| 208 | Pol | 322 | 330 | 9 | ILDPFRKQN |
| 213 | Pol | 333 | 354 | 22 | IVIYQYMDDLYVGSDLEIGQHR |
| 214 | Pol | 333 | 354 | 22 | IVIYQYVDDLYVGSDLEIEQHR |
| 221 | Pol | 367 | 399 | 33 | WGFTTPDKKHQKEPPFLWMGYELHPDKWTVQPI |
| 222 | Pol | 367 | 399 | 33 | WGLTTPDKKHQKDPPFLWMGYELHPDRWTVQPI |
| 236 | Pol | 401 | 431 | 31 | LPEKDSWTVNDIQKLVGKLNWASQIYPGIKV |
| 237 | Pol | 401 | 431 | 31 | LPEKESWTVNDIQKLIGKLNWASQIYAGIKV |
| 245 | Pol | 434 | 442 | 9 | LCKLLRGTK |
| 246 | Pol | 434 | 442 | 9 | LCKLLRGAK |
| 247 | Pol | 453 | 471 | 19 | EAELELAENREILKEPVHG |
| 248 | Pol | 453 | 471 | 19 | EAEIELAENREILREPVHG |
| 249 | Pol | 467 | 478 | 12 | EPVHGVYYDPSK |
| 250 | Pol | 467 | 478 | 12 | EPVHGAYYDPSK |
| 251 | Pol | 490 | 511 | 22 | GQWTYQIYQEPFKNLKTGKYAR |
| 252 | Pol | 490 | 511 | 22 | GQWSYQIYQEPYKNLKTGKYAK |
| 253 | Pol | 515 | 530 | 16 | AHTNDVKQLTEAVQKI |
| 254 | Pol | 515 | 530 | 16 | AHTNDVRQLTEAVQKV |
| 255 | Pol | 535 | 544 | 10 | IVIWGKTPKF |
| 256 | Pol | 535 | 544 | 10 | IVIWGKIPKF |
| 257 | Pol | 542 | 554 | 13 | PKFKLPIQKETWE |
| 258 | Pol | 542 | 554 | 13 | PKFRLPIQKETWD |
| 263 | Pol | 559 | 589 | 31 | EYWQATWIPEWEFVNTPPLVKLWYQLEKEPI |
| 264 | Pol | 559 | 589 | 31 | DYWQATWIPEWEFTNTPPLVKLWYQLETEPI |
| 266 | Pol | 591 | 606 | 16 | GAETFYVDGAANRETK |
| 267 | Pol | 591 | 606 | 16 | GVETFYVDGASNRETK |
| 268 | Pol | 625 | 637 | 13 | TDTTNQKTELQAI |
| 269 | Pol | 625 | 637 | 13 | ADTTNQKTELHAI |
| 270 | Pol | 636 | 644 | 9 | AIHLALQDS |
| 271 | Pol | 636 | 644 | 9 | AIYLALQDS |
| 272 | Pol | 639 | 671 | 33 | LALQDSGLEVNIVTDSQYALGIIQAQPDKSESE |
| 273 | Pol | 639 | 671 | 33 | LALQDSGSEVNIVTDSQYAIGIIQAQPDRSESE |
| 278 | Pol | 673 | 681 | 9 | VSQIIEQLI |
| 279 | Pol | 673 | 681 | 9 | VNQIIEQLI |
| 280 | Pol | 675 | 684 | 10 | QIIEQLIKKE |
| 281 | Pol | 675 | 684 | 10 | QIIEQLINKE |
| 282 | Pol | 683 | 708 | 26 | KEKVYLAWVPAHKGIGGNEQVDKLVS |
| 283 | Pol | 683 | 708 | 26 | KEKIYLAWVPAHKGIGGNEQIDKLVS |
| 284 | Pol | 710 | 725 | 16 | GIRKVLFLDGIDKAQE |
| 285 | Pol | 710 | 725 | 16 | GIRRVLFLDGIEKAQD |
| 286 | Pol | 727 | 735 | 9 | HEKYHSNWR |
| 287 | Pol | 727 | 735 | 9 | HEKYHNNWR |
| 288 | Pol | 737 | 745 | 9 | MASDFNLPP |
| 289 | Pol | 737 | 745 | 9 | MASDFNIPP |
| 290 | Pol | 741 | 753 | 13 | FNLPPVVAKEIVA |
| 291 | Pol | 741 | 753 | 13 | FNLPPIVAKEIVA |
| 292 | Pol | 741 | 827 | 87 | FNLPPVVAKEIVASCDKCQLKGEAMHG |
| | | | | | QVDCSPGIWQLDCTHLEGKIILVAVHVA |
| | | | | | SGYIEAEVIPAETGQETAYFLLKLAGRW |
| | | | | | PVKT |
| 293 | Pol | 741 | 827 | 87 | FNLPPIVAKEIVACCDKCQLKGEAIHGQ |
| | | | | | VDCSPGVWQLDCTHLEGKVILVAVHVA |
| | | | | | SGYIEAEIIPTETGQETAYFILKLAGRWPV |
| | | | | | TT |
| 294 | Pol | 747 | 827 | 81 | VAKEIVASCDKCQLKGEAMHGQVDCSP |
| | | | | | GIWQLDCTHLEGKIILVAVHVASGYIEAE |
| | | | | | VIPAETGQETAYFLLKLAGRWPVKT |
| 295 | Pol | 747 | 827 | 81 | VAKEIVACCDKCQLKGEAIHGQVDCSPG |
| | | | | | VWQLDCTHLEGKVILVAVHVASGYMEA |
| | | | | | EVIPTETGQETAYFILKLAGRWPVTT |
| 305 | Pol | 840 | 919 | 80 | TVKAACWWAGIKQEFGIPYNPQSQGVV |
| | | | | | ESMNKELKKIIGQVRDQAEHLKTAVQM |
| | | | | | AVFIHNFKRKGGIGGYSAGERIVDII |
| 306 | Pol | 840 | 919 | 80 | AVKAACWWAGVKQEFGIPYHPQSQGV |
| | | | | | VESMNNELKKIIGQIRDQAEQLKTAVQM |
| | | | | | AVLIHNFKRKGGIGEYSAGERIIDII |
| 307 | Pol | 840 | 920 | 81 | TVKAACWWAGIKQEFGIPYNPQSQGVV |
| | | | | | ESMNKELKKIIGQVRDQAEHLKTAVQM |
| | | | | | AVFIHNFKRKGGIGGYSAGERIVDIIA |
| 308 | Pol | 840 | 920 | 81 | AVKAACWWAGVKQEFGIPYHPQSQGV |
| | | | | | VESMNNELKKIIGQIRDQAEQLKTAVQM |
| | | | | | AVLIHNFKRKGGIGEYSAGERIIDIIA |
| 309 | Pol | 840 | 1003 | 164 | TVKAACWWAGIKQEFGIPYNPQSQGVV |
| | | | | | ESMNKELKKIIGQVRDQAEHLKTAVQM |
| | | | | | AVFIHNFKRKGGIGGYSAGERIVDIIATDI |
| | | | | | QTKELQKQITKIQNFRVYYRDSRDPLWK |
| | | | | | GPAKLLWKGEGAVVIQDNSDIKVVPRR |
| | | | | | KAKIIRDYGKQMAGDDCVASRQDED |
| 310 | Pol | 840 | 1003 | 164 | AVKAACWWAGVKQEFGIPYNTQSQGV |
| | | | | | VESMNNELKKIIGQIRDQAEHLKTAVQM |
| | | | | | AVLIHNFKRKGGIGEYSAGERIIDIIATDI |
| | | | | | QTRELQKQITKLQNFRVYYRDNRDPLW |
| | | | | | KGPARLLWKGEGAVVIQDNSEIKVVPRR |
| | | | | | KVKIIRDYGKRMAGDDCVAGRQDED |
| 313 | Pol | 917 | 925 | 9 | DIIATDIQT |
| 314 | Pol | 917 | 925 | 9 | DIIASDIQT |
| 315 | Pol | 922 | 930 | 9 | DIQTKELQK |
| 316 | Pol | 922 | 930 | 9 | DIQTRELQK |
| 317 | Pol | 924 | 932 | 9 | QTKELQKQI |
| 318 | Pol | 924 | 932 | 9 | QTRELQKQI |
| 321 | Pol | 932 | 1003 | 72 | ITKIQNFRVYYRDSRDPLWKGPAKLLWK |
| | | | | | GEGAVVIQDNSDIKVVPRRKAKIIRDYG |
| | | | | | KQMAGDDCVASRQDED |
| 322 | Pol | 932 | 1003 | 72 | ITKLQNFRVYYRDNRDPLWKGPARLLW |
| | | | | | KGEGAVVIQDNSEIKVVPRRKVKIIRDY |
| | | | | | GKRMAGDDCVAGRQDED |

With respect to the range of lengths of the individual polypeptide or peptide segments, in various embodiments, each polypeptide segment is at least 8 amino acids in length, and up to about 250 amino acids in length, e.g., from at least 8 amino acids in length up to 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 45, 50, 55, 60, 65, 70, 75, 80, 85, 90, 95, 100, 110, 120, 130, 140, 150, 160, 170, 180, 190, 200, 210, 220, 230, 240 or 250 amino acids in length. In various embodiments, each polypeptide segment is at least 8 amino acids in length, and up to about 35 amino acids in length, e.g., from at least 8 amino acids in length up to 9, 10, 11, 12, 13, 14, 15, 16, 17, 18, 19, 20, 21, 22, 23, 24, 25, 26, 27, 28, 29, 30, 31, 32, 34 or 35 amino acids in length. In various embodiments, each polypeptide segment is at least 15 amino acids in length, and up to about 30 amino acids in length, e.g., from at least 15 amino acids in length up to 16, 17, 18, 19, 20, 21, 22, 23, 24, 25, 26, 27, 28, 29 or 30 amino acids in length.

With respect to the length of the full-length fusion polypeptide, in various embodiments, in some embodiments, the full-length of the fusion polypeptide comprises at least about 350 amino acids and up to about 1000 amino acids, e.g., at least about 350 amino acids and up to about 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 or 1000 amino acids. With respect to the length of the full-length fusion polypeptide, in various embodiments, in some embodiments, the full-length of the fusion polypeptide comprises at least about 350 amino acids and up to about 800 amino acids, e.g., at least about 350 amino acids and up to about 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 670, 680, 690, 700, 710, 720, 730, 740, 750, 760, 770, 780, 790, or 800 amino acids. In some embodiments, the full-length of the fusion polypeptide is no longer than 800 amino acids, e.g., no longer than 795, 790, 785, 780, 775, 770, 765, 760, 755, 750, 745, 740, 735, 730, 725, 720, 715, 710, 705 or 700 amino acids.

Generally, the fusion polypeptides are immunogenic, in that they are capable of eliciting an immune response in a human, e.g., against HIV-1. In some embodiments, the fusion polypeptides, optionally in combination with one or more additional therapeutic agents, e.g., as described herein, are capable of eliciting a protective or a therapeutically effective immune response in a human against HIV-1, e.g., capable of either preventing HIV-1 infection in an uninfected individual, or in therapeutic contexts, capable of eliciting an immune response sufficient to induce immune mediated control of HIV-1 or eradicate HIV-1 in an infected individual. The immunogenicity of the fusion polypeptides can be evaluated and demonstrated, in in vitro and in vivo assays, as described herein. For example, immunogenicity of the fusion polypeptides can be demonstrated by an in vitro assay, including CD4+ and/or CD8+ T-cell activation (e.g., including cytokine expression and target killing assays) or proliferation assays. The T-cells can be activated by exposure to antigen presenting cells (APCs) (such as dendritic cells, e.g., monocyte-derived dendritic cells) that have been transfected with a polynucleotide encoding the fusion polypeptide. Such assays are known in the art and described herein. The immunogenicity of the fusion polypeptides can also be demonstrated in in vivo animal models, for example, by administering to mice, e.g., transgenic for one or more human HLA molecules (available from Jackson Laboratories or Taconic), or non-human primates, and evaluating CD4+ and/or CD8+ T-cell activation (e.g., including serum cytokine levels) or proliferation. In various embodiments, one, two, three, or more, of each polypeptide segment comprises or consists of one or more predicted T cell epitopes, e.g., as computationally or experimentally determined. In some embodiments, the fusion polypeptide comprises one or more polypeptide segments that bind to or are presented by one or more human HLA class I and/or class II alleles (e.g. 1, 2, 3, 4, 5 or 6 alleles), e.g. within a single subject or amongst multiple subjects. In some embodiments, the fusion polypeptide comprises one or more polypeptide segments that bind to or are presented by at least by a human A*0201 HLA class I molecule. In some embodiments, the fusion polypeptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more, 8-mer, 9-mer and/or 10-mer polypeptide segments that bind to or are presented by one or more human HLA class I and/or class II alleles (e.g. 1, 2, 3, 4, 5 or 6 alleles), e.g. within a single subject. In some embodiments, the fusion polypeptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more polypeptide segments, each 15-30 amino acids in length, that are intracellularly processed and presented by one or more human HLA class I and/or class II alleles (e.g. 1, 2, 3, 4, 5 or 6 alleles), e.g. within a single subject.

### Concatenating Polypeptide Segments

As appropriate, the one or more of the polypeptide segments can be directly abutted or fused to an adjacent segment, or can be joined, connected or linked to an adjacent segment by one or more peptide linkers. In various embodiments, the one or more peptide linkers is selected from one or more of a polyalanine linker, a polyglycine linker, a cleavable linker, a flexible linker, a rigid linker, a Nef linking sequence, and combinations thereof, e.g., within a linker or within a full-length fusion polypeptide. Illustrative fusion protein linkers that can be used in the present fusion polypeptides to connect one or more polypeptide segments are described, e.g., in Chen, et al., Adv Drug Deliv Rev. (2013) 65 (10): 1357-1369. In some embodiments, the polyalanine linker comprises or consists of 2 or 3 contiguous alanine residues, e.g. AA, AAA, AAY or AAX, wherein X is any amino acid (e.g., A, C, D, E, F, G, H, I, K, L, M, N, P, Q, R, S, T, V, Y). In some embodiments, a polyglycine linker is used, e.g., GGS, GSG or GGGS (SEQ ID NO:421).

In some embodiments, the cleavable linker is selected from a 2A cleavable peptide. Illustrative 2A cleavable peptides that can be used in the present fusion polypeptides to connect one or more polypeptide segments are described, e.g., in Donnelly, et al., J. Gen. Virol (2001), 82, 1027-1041 and Chng, et al., mAbs (2015) 7:2, 403-412. Illustrative cleavable peptides that can be used to link one or more polypeptide segments include without limitation 2A cleavage sequences (e.g., foot-and-mouth disease virus (F2A), equine rhinitis A virus (E2A), porcine teschovirus-1 (P2A) and Thosea asigna virus (T2A)), and furin recognition/cleavage sequences (e.g. REKR (SEQ ID NO: 382), RRKR (SEQ ID NO: 383), RAKR (SEQ ID NO: 381)). In certain embodiments, a furin recognition/cleavage sequence (e.g., REKR (SEQ ID NO: 382), RRKR (SEQ ID NO: 383), RAKR (SEQ ID NO: 381)) is combined or fused with a 2A cleavable peptide (e.g., foot-and-mouth disease virus (F2A), equine rhinitis A virus (E2A), porcine teschovirus-1 (P2A) and Thosea asigna virus (T2A)) in a single linker. See, e.g., Chng, et al., mAbs (2015) 7:2, 403-412. In various embodiments, the 2A cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), NO: 385), APVKQTLNFDLLKLAGDVESNPGP (SEQ ID RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388), or comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388). As appropriate, in certain embodiments, a furin recognition/cleavage sequence can be positioned either at the N-terminus or the C-terminus of a 2A linker. In some embodiments, the cleavable linker comprises or consists of a furin recognition/cleavage site selected from the group consisting of RAKR (SEQ ID NO: 381), REKR (SEQ ID NO: 382) and RRKR (SEQ ID NO: 383). REKR (SEQ ID NO: 382) is a naturally occurring cleavable linker in HIV and SIV envelope glycoprotein precursor (Bahbouhi, et al., Biochem. J. (2002) 366, 863-872). In some embodiments, the fusion polypeptide comprises one or more Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), or comprises or consists of an amino acid sequence selected from VHAGPIA (SEQ ID NO: 389), VHAGPVA (SEQ ID NO: 390) and GALDI (SEQ ID NO: 391). Illustrative linkers that can be used to link or connect one or more polypeptide segments in a fusion polypeptide are provided in Table 4.

TABLE 4
illustrative linkers for connecting polypeptide segments

| SEQ ID NO: | NAME | SEQUENCE |
|---|---|---|
| | poly-alanine (2) | AA |
| | poly-alanine (3) | AAA |
| | poly-alanine-Tyr | AAY |
| | poly-alanine-XXX | AAX (X = any amino acid) |
| 381 | furin recognition site | RAKR |
| 382 | furin recognition site | REKR |
| 383 | furin recognition site | RRKR |
| 384 | P2A | ATNFSLLKQAGDVEENPGP |
| 385 | F2A | APVKQTLNFDLLKLAGDVESNPGP |
| 386 | F2A + furin recognition site | RAKRAPVKQTLNFDLLKLAGDVESNPGP |
| 387 | E2A | QCTNYALLKLAGDVESNPGP |
| 388 | T2A | EGRGSLLTCGDVEENPGP |
| 389 | Nef natural sequence link | VHAGPIA |
| 390 | Nef natural sequence link | VHAGPVA |
| 391 | Nef natural sequence link | GALDI |
| 392 | Nef natural sequence link | GALDL |
| | poly-glycine | GGS |
| | poly-glycine | GSG |
| 421 | Gly3Ser | GGGS |

### Polypeptide Segments Encoded by HIV-1 Gag Gene

In various embodiments, the fusion polypeptide comprises one or more segments of one or more viral proteins, or fragments or subsequences thereof, encoded by the HIV-1 Gag gene. In some embodiments, the one or more viral proteins encoded by the HIV-1 Gag gene is selected from p17 (N-terminal matrix), p24 (capsid), p7 (nucleocapsid) and p6 (C-terminus). In some embodiments, the one or more viral proteins encoded by the HIV-1 Gag gene does not comprise any p6 components. In some embodiments, the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 68-146 and 339-342; SEQ ID NOs: 68, 69, 72, 73, 74, 75, 76, 77, 80, 81, 82, 83, 84, 85, 86, 87, 88, 89, 92, 93, 101, 102, 103, 104, 109, 110, 115, 116, 125, 126, 127, 128, 129, 130, 131, 132, 133, 134, 135, 136, 139, 140, 141, 142, 143, 144, 145 and 146; SEQ ID NOs: 76, 77, 86, 87 and 92-124; SEQ ID NOs: 76, 77, 86, 87, 94 and 95; SEQ ID NOs: 76, 86 and 94; SEQ ID NOs: 77, 87 and 95; SEQ ID NOs: 68-79 and 92-124; SEQ ID NOs: 70-71, 76-77 and 94-95; SEQ ID NOs: 78, 79, 96, 99, 100, 107, 108, 113, 114, 121, 122, 123, 124, 137 and 138; SEQ ID NOs: 78, 99, 107, 113, 121, 123 and 137; SEQ ID NOs: 78, 79, 90, 91, 97, 98, 99, 100, 105, 106, 107, 108, 111, 112, 113, 114, 117, 118, 119, 120, 121, 122, 123, 124, 137 and 138; SEQ ID NOs: 78, 90, 97, 105, 111, 117, 119 and 137; and SEQ ID NOs: 78 and 137.

In some embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, or more, segments comprising or consisting of an HIV-1 Gag amino acid sequence corresponding to amino acid residue positions selected from 31-53, 37-51, 142-166, 175-199, 183-191, 257-282, 257-290, 265-282, 288-313, 288-321, 296-313, 333-357, 337-361, 341-349, 345-353 and 429-444, wherein the amino acid positions are with respect to SEQ ID NO:404. In certain embodiments, the fusion polypeptide does not comprise 1, 2, 3, 4, 5, or more, polypeptide segments comprising or consisting of an HIV-1 Gag amino acid sequence corresponding to amino acid residue positions selected from 1-30, 54-127, 138-146, 370-428 and 445-500, or subsequences thereof, wherein the amino acid positions are with respect to SEQ ID NO:404. In some embodiments, the plurality of polypeptide segments does not, or the herein described fusion proteins do not, comprise 1, 2, 3, 4, 5, or more, polypeptide segments comprising or consisting of an HIV-1 Gag amino acid sequence of any one of SEQ ID NOs: 444-448, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 444-448, or subsequences thereof. As used herein, numbering of a given amino acid polymer or nucleic acid polymer “corresponds to”, is “corresponding to” or is “relative to” the numbering of a selected or reference amino acid polymer or nucleic acid polymer when the position of any given polymer component (e.g., amino acid, nucleotide, also referred to generically as a “residue”) is designated by reference to the same or to an equivalent position (e.g., based on an optimal alignment or a consensus sequence) in the selected amino acid or nucleic acid polymer, rather than by the actual numerical position of the component in the given polymer.

### Polypeptide Segments Encoded by HIV-1 Nef Gene

In some embodiments, the fusion polypeptide comprises one or more segments of the viral protein encoded by the HIV-1 Nef gene. In some embodiments, the plurality of polypeptide segments comprises at least one polypeptide segment, e.g., at least 2, 3, 4, 5, 6, 7, 8, 9, 10, 11, 12, 13, 14, 15, 16, 17, 18, 19, 20, 21, 22, 23, 24, 25, or more, segments comprising or consisting of an amino acid sequence selected from: SEQ ID NOs: 147, 148, 149, 150, 151, 152, 153, 154, 155, 156, 157, 158, 159, 160, 161, 162, 163, 164, 165, 166, 167, 168, 169, 170, 171 and 172; SEQ ID NOs: 147, 148, 149, 150, 155, 156, 157, 158, 159, 160, 166, 167, 168, 169, 170 and 171; SEQ ID NOs: 149-152; SEQ ID NOs: 151-152; SEQ ID NOs: 149, 150, 151, 152, 159, 160, 161, 162, 163, 164, 166, 167, 168, 169, 170, 171, 172, 173 and 174; SEQ ID NOs: 151, 152, 161 and 162; SEQ ID NOs: 151 and 152; SEQ ID NOs: 153, 154, 172 and 173; SEQ ID NOs: 153 and 172; SEQ ID NOs: 153, 154, 155, 156, 157, 158, 163, 164, 165, 166, 167, 168, 169, 170, 171, 172 and 173; SEQ ID NOs: 153 and 165; and SEQ ID NO: 153.

In some embodiments, the fusion polypeptide comprises at least 2 polypeptide segments, e.g., at least 3, 4, 5, 6, 7, 8, 9, 10, 11, 12, 13, 14, 15, or more, segments comprising or consisting of an HIV-1 Nef amino acid sequence corresponding to amino acid residue positions selected from 64-102, 81-102, 88-97, 91-99, 130-148, 130-154, 134-142, 134-148, 136-148, 137-145, 137-145 and 117-154, wherein the amino acid positions are with respect to SEQ ID NO:405. In certain embodiments, the fusion polypeptide does not comprise 1, 2, 3, or more, polypeptide segments comprising or consisting of an HIV-1 Nef amino acid sequence corresponding to amino acid residue positions selected from 1-63, 103-116 and 155-206, or subsequences thereof, wherein the amino acid positions are with respect to SEQ ID NO:405. In some embodiments, the plurality of polypeptide segments does not, or the herein described fusion proteins do not, comprise 1, 2, 3, or more, polypeptide segments comprising or consisting of an HIV-1 Nef amino acid sequence of any one of SEQ ID NOs: 449-451, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 449-451, or subsequences thereof.

### Fusion Polypeptides Having Polypeptide Segments Encoded by HIV-1 Gag and Nef Genes

In some embodiments, the fusion polypeptide comprises or consists of one or more segments of viral proteins encoded by the HIV-1 Gag and Nef genes, e.g., does not comprise one or more polypeptide segments encoded by the HIV-1 Env, Pol, Tat, Rev, Vif, Vpr or Vpu genes. In some embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 68-79 and 92-124, 149, 150, 151, 152, 159, 160, 161, 162, 163, 164, 166, 167, 168, 169, 170, 171, 172, 173 and 174; SEQ ID NOs: 70, 71, 76, 77, 94, 95, 151, 152, 161 and 162; SEQ ID NOs: 70, 76, 94, 151 and 161; and SEQ ID NOs: 71, 77, 95, 152 and 162.

In some embodiments, the fusion polypeptide comprises or consists of the following polypeptide segments in sequential order, from N-terminus to C-terminus, optionally joined or connected by one or more linkers: SEQ ID NOs: 70, 76, 94, 151 and 161; or SEQ ID NOs: 71, 77, 95, 152 and 162.

In some embodiments, the fusion polypeptide comprises or consists of an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430.

Modifications may be made in the structure of the fusion polypeptides and polynucleotides encoding such fusion polypeptides, described herein, and still obtain a functional molecule that encodes a variant or derivative polypeptide with desirable (e.g., immunogenic) characteristics. When it is desired to alter the amino acid sequence of a polypeptide to create an equivalent, or even an improved, variant or portion of a fusion polypeptide described herein, one skilled in the art will typically change one or more of the codons of the encoding DNA sequence.

For example, certain amino acids may be substituted for other amino acids in a protein structure without appreciable loss of its ability to bind other polypeptides (e.g., antigens) or cells. Since it is the binding capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the polypeptide sequences of the disclosed fusion polypeptides, or corresponding DNA sequences that encode such fusion polypeptides without appreciable loss of their biological utility or activity.

In many instances, a polypeptide variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins-Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J. (1990) Unified Approach to Alignment and Phylogenes pp. 626-645 Methods in Enzymology vol. 183, Academic Press, Inc., San Diego, CA; Higgins, D. G. and Sharp, P. M. (1989) CABIOS 5:151-153; Myers, E.W. and Muller W. (1988) CABIOS 4:11-17; Robinson, E. D. (1971) Comb. Theor 77:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, CA; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides described herein. Software for performing BLAST analyses is publicly available through the National Center for Biotechnology Information (blast.ncbi.nlm.nih.gov/Blast.cgi).

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

A “polypeptide variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences described herein and evaluating one or more biological activities of the polypeptide as described herein and/or using any of a number of techniques well known in the art. The term “variant” may also refer to any naturally occurring or engineered molecule comprising one or more nucleotide or amino acid mutations.

### Polypeptide Segments Encoded by HIV-1 Env Gene

In some embodiments, the fusion polypeptides comprise one or more segments of one or more viral proteins encoded by the HIV-1 Env gene. In certain embodiments, the one or more viral proteins encoded by the HIV-1 Env gene is selected from gp120 and gp41.

In various embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 1-67 and 338; SEQ ID NOs: 2, 3, 8, 9, 13, 14, 17, 18, 23, 24, 25, 26, 28, 29, 31, 32, 33, 34, 35, 36, 37, 38, 39, 40, 43, 44, 45, 46, 47, 48, 49, 50, 51, 52, 53, 54, 55, 56, 58, 59, 62, 63, 64, 65, 66 and 67; SEQ ID NOs: 4, 5, 6, 7, 11, 12, 13, 14, 15, 16, 28, 29, 30, 37, 38, 41, 42, 43, 44, 45, 46, 47, 48, 49, 50, 51, 52, 53, 54, 55, 56, 57, 58, 59, 60, 61 and 338; SEQ ID NOs: 4, 5, 6, 7, 11, 12, 13, 14, 15, 16, 28, 29, 30, 37, 38, 41 and 42; SEQ ID NOs: 28, 29, 30 and 41-56; SEQ ID NOs: 28, 29, 41 and 42; SEQ ID NOs: 4, 5, 6, 7, 11, 12, 13, 14, 15, 16, 37 and 38; SEQ ID NOs: 4, 5, 11, 12, 37 and 38; SEQ ID NOs: 6, 7, 15, 16, 21, 22, 30, 60 and 61; SEQ ID NOs: 6, 15, 21, 30 and 60; SEQ ID NOs: 1, 2, 3, 4, 5, 6, 7, 10, 11, 12, 13, 14, 15, 16, 19, 20, 27, 55, 56, 57, 58, 59, 60, 61 and 338; SEQ ID NOs: 1, 10, 19, 27, 55, 56 and 57; and SEQ ID NOs: 6, 15 and 60.

In various embodiments, the fusion polypeptide comprises at least 2 polypeptide segments, e.g., at least 3, 4, 5, 6, 7, 8, 9, 10, 11, 12, 13, 14, or more, segments comprising or consisting of an HIV-1 Env amino acid sequence corresponding to amino acid residue positions selected from 28-52, 34-48, 34-47, 36-44, 59-83, 64-83, 66-83, 67-75, 113-137, 235-259, 586-594, 586-610, 589-606 and 594-602, wherein the amino acid positions are with respect to SEQ ID NO: 403. In certain embodiments, the fusion polypeptide does not comprise 1, 2, 3, 4, 5, 6, or more, polypeptide segments comprising or consisting of an HIV-1 Env amino acid sequence corresponding to amino acid residue positions selected from 1-27, 53-58, 84-112, 138-234, 269-474, 490-501, 611-856, or subsequences thereof, wherein the amino acid positions are with respect to SEQ ID NO:403. In some embodiments, the plurality of polypeptide segments does not, or the herein described fusion proteins do not, comprise 1, 2, 3, 4, 5, or more, polypeptide segments comprising or consisting of an HIV-1 Env amino acid sequence of any one of SEQ ID NOs: 437-443, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 437-443, or subsequences thereof.

### Polypeptide Segments Encoded by HIV-1 Pol Gene

In some embodiments, the fusion polypeptides comprise of one or more segments of one or more viral proteins encoded by the HIV-1 Pol gene. In various embodiments, the one or more viral proteins encoded by the HIV-1 Pol gene is selected from one or more of protease (PR), reverse transcriptase (RT), and integrase (INT).

In some embodiments, the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 174-337 and 343-344; SEQ ID NOs: 174, 175, 178, 179, 180, 181, 182, 183, 184, 185, 193, 194, 195, 196, 197, 198, 199, 200, 203, 204, 205, 206, 207, 208, 213, 214, 221, 222, 236, 237, 245, 246, 247, 248, 249, 250, 251, 252, 253, 254, 255, 256, 257, 258, 263, 264, 266, 267, 268, 269, 270, 271, 272, 273, 278, 279, 280, 281, 282, 283, 284, 285, 286, 287, 288, 289, 290, 291, 292, 293, 294, 295, 305, 306, 307, 308, 309, 310, 313, 314, 315, 316, 317, 318, 321, and 322; SEQ ID NOs: 180, 181, 182, 183, 184, 185, 186, 187, 190, 191, 192, 193, 194, 195, 196, 221, 222, 294, 295, 296, 297, 298, 299, 300, 301, 305, 306, 307, 308, 311, 312, 321, 322, 323, 324, 325, 326, 327, 328, 329, 330, 331, 332, 333, 334, 335, 336 and 337; SEQ ID NOs: 180, 181, 186, 187, 221, 222, 294, 295, 307, 308, 321 and 322; SEQ ID NOs: 176, 177, 178, 179, 180, 181, 182, 183, 184, 185, 186, 187, 188, 189, 190, 191, 192, 193, 194, 195, 196, 197, 198, 199, 200, 201, 202, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 257, 258, 259, 260, 261, 262, 263, 264, 265, 266, 267, 282, 283, 294, 295, 296, 297, 298, 299, 300, 301, 302, 305, 306, 319, 320, 321, 322, 323, 324, 325, 326, 327, 328, 329, 330, 331, 332, 333, 334, 335, 336 and 337; SEQ ID NOs: 176, 177, 188, 189, 213, 214, 223, 224, 259, 260, 282, 283, 294, 295, 305, 306, 319, and 320; SEQ ID NOs: 180, 181, 186, 187, 221, 222, 294, 295, 321 and 322; SEQ ID NOs: 182-202, 292-302, 305, 306; SEQ ID NOS: 188, 189, 294, 295, 305, 306; SEQ ID NOs: 176, 177, 178, 179, 180, 181, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 257, 258, 259, 260, 261, 262, 263, 264, 265, 266, 267, 282, 283, 319, 320, 321, 322, 323, 324, 325, 326, 327, 328, 329, 330, 331, 332, 333, 334, 335, 336 and 337; SEQ ID NOs: 176, 177, 213, 214, 223, 224, 259, 260, 282, 283, 319 and 320; SEQ ID NOs: 192, 201, 202, 215, 216, 217, 218, 219, 220, 229, 230, 231, 240, 241, 242, 243, 244, 265, 276, 277, 298, 299, 302, 311, 312, 327, 328, 331, 332, 333, 336 and 337; SEQ ID NOs: 192, 201, 215, 217, 219, 229, 230, 240, 241, 243, 265, 276, 298, 302, 311, 327, 331, 333 and 336; SEQ ID NOs: 190, 191, 192, 197, 198, 209, 210, 211, 212, 213, 214, 215, 216, 217, 218, 219, 220, 225, 226, 227, 228, 229, 230, 231, 232, 233, 234, 235, 238, 239, 261, 262, 274, 275, 276, 277, 296, 297, 298, 299, 300, 301, 302, 303, 304, 323, 324, 325, 326, 327, 328, 329, 330, 331, 332, 333, 334, 335, 336, 337, 343, 344, 375 and 376; SEQ ID NOs: 190, 197, 209, 210, 211, 225, 227, 234, 238, 261, 296, 300, 303, 323, 325, 329, 334; and SEQ ID NOs: 192, 215, 217, 219, 229, 230, 276, 298, 302, 327, 331, 333 and 336.

In some embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 39, 30, or more, segments comprising or consisting of an HIV-1 Pol amino acid sequence corresponding to amino acid residue positions selected from 144-168, 152-160, 291-315, 326-350, 328-352, 330-354, 333-354, 334-342, 336-344, 338-346, 374-398, 380-404, 382-390, 388-396, 399-423, 400-424, 406-430, 553-577, 642-666, 650-658, 759-783, 767-775, 768-792, 776-784, 834-858, 940-964, 947-971, 948-956, 948-972, 955-963, 956-964, 980-1003 and 988-996, wherein the amino acid positions are with respect to SEQ ID NO:406. In certain embodiments, the fusion polypeptide does not comprise 1, 2, 3, 4, 5, 6, 7, 8, 9, 10, or more, polypeptide segments comprising or consisting of an HIV-1 Pol amino acid sequence corresponding to amino acid residue positions selected from 1-55, 118-128, 321-325, 355-366, 432-541, 607-641, 667-682, 709-746, 828-833, 921-930, or subsequences thereof, wherein the amino acid positions are with respect to SEQ ID NO: 406. In some embodiments, the plurality of polypeptide segments does not, or the herein described fusion proteins do not, comprise 1, 2, 3, 4, 5, or more, polypeptide segments comprising or consisting of an HIV-1 Pol amino acid sequence of any one of SEQ ID NOs: 452-461, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 452-461, or subsequences thereof.

In some embodiments, a fusion polypeptide comprising polypeptide segments encoded by the HIV-1 Pol gene does not comprise the amino acid sequence or motif YMDD (SEQ ID NO: 462) or YVDD (SEQ ID NO: 463). In some embodiments, the fusion polypeptide does not comprise one or more amino acid sequences selected from SEQ ID NOs: 215, 216, 217, 218, 219 and 220. In some embodiments, the fusion polypeptide does not comprise one or more amino acid sequences selected from SEQ ID NOs: 209, 210, 211, 212, 213, 214, 343 and 344.

### Fusion Polypeptides Having Polypeptide Segments Encoded by HIV-1 Env and Pol Genes

In some embodiments, the fusion polypeptide comprises or consists of one or more segments of viral proteins encoded by the HIV-1 Env and Pol genes, e.g., does not comprise one or more polypeptide segments encoded by the HIV-1 Gag, Nef, Tat, Rev, Vif, Vpr or Vpu genes.

In some embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 4, 5, 6, 7, 11, 12, 13, 14, 15, 16, 28, 29, 30, 37, 38, 41, 42, 43, 44, 45, 46, 47, 48, 49, 50, 51, 52, 53, 54, 55, 56, 57, 58, 59, 60, 61, 176, 177, 178, 179, 180, 181, 182, 183, 184, 185, 186, 187, 188, 189, 190, 191, 192, 193, 194, 195, 196, 197, 198, 199, 200, 201, 202, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 257, 258, 259, 260, 261, 262, 263, 264, 265, 266, 267, 282, 283, 294, 295, 296, 297, 298, 299, 300, 301, 302, 305, 306, 319, 320, 321, 322, 323, 324, 325, 326, 327, 328, 329, 330, 331, 332, 333, 334, 335, 336, 337 and 338; SEQ ID NOs: 4, 5, 6, 7, 11, 12, 13, 14, 15, 16, 28, 29, 30, 37, 38, 41, 42, 176, 177, 188, 189, 213, 214, 223, 224, 259, 260, 282, 283, 294, 295, 305, 306, 319 and 320; SEQ ID NOs: 28, 29, 30, 41-56, 182-202, 292-302, 305 and 306; SEQ ID NOs: 28, 29, 41, 42, 188, 189, 294, 295, 305 and 306; SEQ ID NOs: 4, 5, 6, 7, 11, 12, 13, 14, 15, 16, 37, 38, 176, 177, 178, 179, 180, 181, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 257, 258, 259, 260, 261, 262, 263, 264, 265, 266, 267, 282, 283, 319, 320, 321, 322, 323, 324, 325, 326, 327, 328, 329, 330, 331, 332, 333, 334, 335, 336 and 337; and SEQ ID NOs: 4, 5, 11, 12, 37, 38, 176, 177, 213, 214, 223, 224, 259, 260, 282, 283, 319 and 320.

In some embodiments, the fusion polypeptide comprises the following polypeptide segments in sequential order, from N-terminus to C-terminus, optionally joined or connected by one or more linkers: SEQ ID NOs: 188, 305, 28, 41, 294, 4, 176, 11, 319, 259, 282, 223, 213 and 37; SEQ ID NOs: 188, 305, 28, 41 and 294; SEQ ID NOs: 4, 176, 11, 319, 259, 282, 223, 213 and 37; SEQ ID NOs: 189, 306, 29, 42, 295, 5, 177, 12, 320, 260, 283, 224, 214 and 38; SEQ ID NOs: 189, 306, 29, 42 and 295; SEQ ID NOs: 5, 177, 12, 320, 260, 283, 224, 214 and 38; SEQ ID NOs: 305, 319, 259, 282, 223, 213, 294, 176 and 188; SEQ ID NOs: 306, 320, 260, 283, 224, 214, 295, 177 and 189; SEQ ID NOs: 305, 294, 223, 213, 176, 259, 319, 188 and 282; SEQ ID NOs: 306, 295, 224, 214, 177, 260, 320, 189 and 283; SEQ ID NOs: 305, 294, 319, 259, 282, 223, 176, and 188; SEQ ID NOs: 306, 295, 320, 260, 283, 224, 177 and 189; SEQ ID NOs: 305, 223, 294, 176, 259, 319, 188 and 282; or SEQ ID NOs: 306, 224, 295, 177, 260, 320, 189 and 283.

In some embodiments, the fusion polypeptide comprises or consists of an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410, wherein percent identity is across the full length of SEQ ID NOs: 357-366 and 407-410.

### Fusion Polypeptides Having Polypeptide Segments Encoded by HIV-1 Gag, Nef and Pol Genes

In some embodiments, the fusion polypeptide comprises or consists of one or more segments of viral proteins encoded by the HIV-1 Gag, Nef and Pol genes, e.g., does not comprise one or more polypeptide segments encoded by the HIV-1 Env, Tat, Rev, Vif, Vpr or Vpu genes.

In some embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 76, 77, 86, 87, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 149, 150, 151, 152, 180, 181, 182, 183, 184, 185, 186, 187, 190, 191, 192, 193, 194, 195, 196, 221, 222, 294, 295, 296, 297, 298, 299, 300, 301, 305, 306, 307, 308, 311, 312, 321, 322, 323, 324, 325, 326, 327, 328, 329, 330, 331, 332, 333, 334, 335, 336, 337, 339, 340, 341 and 342; and SEQ ID NOs: 76, 77, 86, 87, 94, 95, 151, 152, 181, 182, 186, 187, 221, 222, 294, 195, 307, 308, 321, 322.

In some embodiments, the fusion polypeptide comprises the following polypeptide segments in sequential order, from N-terminus to C-terminus, optionally joined or connected by one or more linkers: SEQ ID NOs: 76, 86, 94, 180, 186, 221, 294, 307, 321 and 151; or SEQ ID NOs: 77, 87, 95, 181, 187, 222, 295, 308, 322 and 152.

In some embodiments, the fusion polypeptide comprises or consists of an amino acid sequence of any one of SEQ ID NOs: 345-350 and 422-424, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 345-350 and 422-424, wherein percent identity is across the full length of SEQ ID NOs: 345-350 and 422-424.

In some embodiments, the fusion polypeptide comprises or consists of an amino acid sequence of any one of SEQ ID NOs: 527-528. In some embodiments, the fusion polypeptide comprises or consists of an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 527-528, wherein percent identity is across the full length of SEQ ID NOs: 527-528.

### Fusion Polypeptides Having Polypeptide Segments Encoded by HIV-1 Env, Gag, Nef and Pol, and Predicted to Bind to or be Presented by Human HLA A*0201 Molecules

As described herein, we have designed fusion polypeptides having a plurality of polypeptide segments that have been computationally predicted to bind to or be presented by human HLA A*0201 molecules. Generally, the selected or included polypeptide segments in such fusion polypeptides comprise polypeptide segments encoded by HIV-1 Env, Gag, Nef and Pol genes (e.g., do not comprise one or more polypeptide segments encoded by the HIV-1 Tat, Rev, Vif, Vpr or Vpu genes), and which are predicted to bind to a human HLA A*0201 molecule with an IC50 value of less than about 1,000 nM. In certain embodiments, the selected or included polypeptide segments in such fusion polypeptides comprise polypeptide segments encoded by HIV-1 Env, Gag, Nef and Pol genes (e.g., do not comprise one or more polypeptide segments encoded by the HIV-1 Tat, Rev, Vif, Vpr or Vpu genes), and which are predicted to bind to a human HLA A*0201 molecule with a percentile rank within the top 5% in a population of polypeptide segments. In some embodiments, the fusion polypeptide comprises or consists of segments of viral proteins encoded by Gag, Pol, Env, and Nef genes, wherein each of the plurality of polypeptide segments can bind to or be presented by a human HLA allele A*0201.

In some embodiments, each of the plurality of polypeptide segments are from 8-35 amino acids in length, e.g. from 9-34 amino acids in length, e.g. from 9-25 amino acids in length.

In some embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 6, 7, 15, 16, 21, 22, 30, 60, 61, 78, 79, 96, 99, 100, 107, 108, 113, 114, 121, 122, 123, 124, 137, 138, 153, 154, 172, 173, 192, 201, 202, 215, 216, 217, 218, 219, 220, 229, 230, 231, 240, 241, 242, 243, 244, 265, 276, 277, 298, 299, 302, 311, 312, 327, 328, 331, 332, 333, 336, and 337; SEQ ID NOs: 6, 15, 21, 30, 60, 78, 99, 107, 113, 121, 123, 137, 153, 172, 192, 201, 215, 217, 219, 229, 230, 240, 241, 243, 265, 276, 298, 302, 311, 327, 331, 333 and 336; SEQ ID NOs: 1, 2, 3, 4, 5, 6, 7, 10, 11, 12, 13, 14, 15, 16, 19, 20, 27, 55, 56, 57, 58, 59, 60, 61, 78, 79, 90, 91, 97, 98, 99, 100, 105, 106, 107, 108, 111, 112, 113, 114, 117, 118, 119, 120, 121, 122, 123, 124, 137, 138, 153, 154, 155, 156, 157, 158, 163, 164, 165, 166, 167, 168, 169, 170, 171, 172, 173, 190, 191, 192, 197, 198, 209, 210, 211, 212, 213, 214, 215, 216, 217, 218, 219, 220, 225, 226, 227, 228, 229, 230, 231, 232, 233, 234, 235, 238, 239, 261, 262, 274, 275, 276, 277, 296, 297, 298, 299, 300, 301, 302, 303, 304, 323, 324, 325, 326, 327, 328, 329, 330, 331, 332, 333, 334, 335, 336, 337, 338, 343 and 344; SEQ ID NOs: 1, 10, 19, 27, 55, 56, 57, 78, 90, 97, 105, 111, 117, 119, 137, 153, 165, 190, 197, 209, 210, 211, 225, 227, 234, 238, 261, 296, 300, 303, 323, 325, 329 and 334.

In some embodiments, the fusion polypeptide comprises the following polypeptide segments in sequential order, from N-terminus to C-terminus, optionally joined or connected by one or more linkers: SEQ ID NOs: 201, 78, 107, 96, 229, 172, 327, 6, 333, 243, 331, 192, 265, 311, 137, 15, 123, 30, 336, 302, 153, 219, 298, 121, 230, 240, 60, 241, 276, 113, 99, 21, 217 and 215; SEQ ID NOs: 78, 296, 1, 339, 197, 329, 232, 323, 303, 234, 90, 261, 274, 238, 211, 325, 137, 227, 209, 190, 341, 57, 225, 27, 210, 119, 19, 165, 334, 117, 153, 10, 97 and 300; or SEQ ID NOs: 296, 1, 78, 197, 339, 227, 261, 274, 238, 325, 137, 329, 303, 234, 90, 232, 27, 57, 225, 323, 190, 341, 119, 19, 165, 334, 117, 153, 10, 97 and 300.

In some embodiments, the fusion polypeptide comprises an amino acid sequence of any one of SEQ ID NOs: 367-377 and 411, or a sequence that is 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 367-377 and 411, wherein percent identity is across the full length of SEQ ID NOs: 367-377 and 411.

### Signal or Leader Sequences

In various embodiments, the fusion polypeptides comprise a signal sequence or signal peptide, e.g., to direct intracellular trafficking of the fusion polypeptide to a proteasomal or lysosomal compartment. In various embodiments, fusion polypeptide comprises a signal sequence at the N-terminus and/or the C-terminus. In some embodiments, the fusion polypeptide comprises an N-terminal signal peptide or leader sequence. In various embodiments, the signal peptide or leader sequence is from a source protein selected from a serum protein, a cytokine, a chemokine, a chaperone protein, an invariant protein, and a protein that directs proteins to the lysosomal compartment. In some embodiments, the signal peptide or leader sequence is from a source protein selected from the group consisting of colony stimulating factor 2 (CSF2, GM-CSF), tissue type plasminogen activator (PLAT, t-PA), C—C motif chemokine ligand 7 (CCL7, MCP-3), C—X—C motif chemokine ligand 10 (CXCL10, IP-10), catenin beta 1 (CTNNB1), CD74 (p33; DHLAG; HLADG; Ia-GAMMA, invariant chain), serum albumin (ALB), polyubiquitin B/C (UBB/UBC), calreticulin (CALR), vesicular stomatitis virus G protein (VSV-G), lysosomal associated membrane protein 1 (LAMP-1) and lysosomal associated membrane protein 2 (LAMP-2). In certain embodiments, the fusion polypeptide comprises N-terminal and C-terminal signal sequences from LAMP-1, e.g, SEQ ID NOs: 399 and 412, respectively. In various embodiments, the signal peptide or leader sequence is selected from an amino acid sequence of any one of SEQ ID NOs: 393-402 and 412-413, or a sequence that is at least 95%, 96%, 97%, 98%, or 99% identical to any one of SEQ ID NOs: 393-402 and 412-413, wherein percent identity is across the full length of SEQ ID NOs: 393-402 and 412-413.

### Polynucleotides Encoding the Fusion Polypeptides

Provided are polynucleotides encoding the fusion polypeptides (e.g., HIV immunogenic polypeptides), described herein, expression cassettes, expression vectors, and viral vectors comprising such polynucleotides, and host cells (e.g., human cells, mammalian cells, yeast cells, plant cells, insect cells, bacterial cells, e.g., E. coli) comprising such polynucleotides, expression cassettes, expression vectors, or viral vectors. As used herein, the phrase “HIV immunogen nucleic acid sequence” refers to polynucleotides encoding HIV immunogenic polypeptides (e.g., fusion polypeptides).

Provided herein are polynucleotides comprising nucleotide sequence(s) encoding any of the fusion polypeptides provided herein, as well as expression cassettes, expression vector(s), and viral vector(s) comprising such polynucleotide sequences, e.g., expression vectors for their efficient expression in host cells, e.g., mammalian cells. In various embodiments, the polynucleotide is a DNA, a cDNA, an mRNA, a self-amplifying RNA (SAM), a self-replicating RNA, or a self-amplifying replicon RNA (RepRNA). In some embodiments, the polynucleotide comprises an alphavirus self-replicating or self-amplifying replicon RNA (RepRNA). Self-replicating RNA and self-amplifying replicon RNA as modes of vaccine delivery are described, e.g., by Tews, et al., Methods Mol Biol. (2017) 1499:15-35; Démoulins, et al., Methods Mol Biol. (2017) 1499:37-75; Englezou, et al., Mol Ther Nucleic Acids. (2018) 12:118-134; McCollough, et al., Vaccines (Basel). (2014) 2 (4): 735-54; and McCollough, et al., Mol Ther Nucleic Acids. (2014) 3: e173.

The terms “polynucleotide” and “nucleic acid molecule” interchangeably refer to a polymeric form of nucleotides and includes both sense and anti-sense strands of RNA, cDNA, genomic DNA, and synthetic forms and mixed polymers of the above. As used herein, the term nucleic acid molecule may be interchangeable with the term polynucleotide. In some embodiments, a nucleotide refers to a ribonucleotide, deoxynucleotide or a modified form of either type of nucleotide, and combinations thereof. The terms also include without limitation, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-biased polynucleotides for improved expression in a desired viral expression vector or host cell.

A “substitution,” as used herein, denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location. “Isolated nucleic acid encoding an polypeptide segment or encoding a fusion polypeptide” refers to one or more nucleic acid molecules encoding such polypeptide segments or fusion polypeptides, including such nucleic acid molecule(s) in a single vector or separate vectors, and such nucleic acid molecule(s) present at one or more locations in a host cell.

A “polynucleotide variant,” as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences described herein and evaluating one or more biological activities of the encoded polypeptide as described herein and/or using any of a number of techniques well known in the art.

In some embodiments, the nucleic acid molecule is codon-biased to enhance expression in a desired host cell, e.g., in human cells, mammalian cells, yeast cells, plant cells, insect cells, or bacterial cells, e.g., E. coli cells. Accordingly, provided are polynucleotides encoding a fusion polypeptide, described herein, wherein the polynucleotides are codon-biased, comprise replacement heterologous signal sequences, and/or have mRNA instability elements eliminated. Methods to generate codon-biased nucleic acids can be carried out by adapting the methods described in, e.g., U.S. Pat. Nos. 5,965,726; 6,174,666; 6,291,664; 6,414,132; and 6,794,498. Preferred codon usage for expression of the fusion polypeptides comprising HIV-1 polypeptide segments from desired viral expression vectors and/or in desired host cells is provided, e.g., at kazusa.or.jp/codon/; and genscript.com/tools/codon-frequency-table.

In some embodiments, the polynucleotide encoding a fusion polypeptide, as described herein, has at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence selected from the group consisting of SEQ ID NOs: 414-418, wherein percent identity is across the full length of SEQ ID NOs: 414-418.

In some embodiments, the polynucleotide encoding a fusion polypeptide, as described herein, has at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence selected from the group consisting of SEQ ID NOs: 524-526, wherein percent identity is across the full length of SEQ ID NOs: 524-526.

In various embodiments, one or more polynucleotides that encode one or more fusion proteins comprising an amino acid sequence of any one of any one of SEQ ID NOs: 345-377, 407-411, 422-424, and 430-435, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 345-377, 407-411, 422-424, and 430-435.

In various embodiments, two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

In various embodiments, the polynucleotide encodes a fusion protein that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of SEQ ID NO: 527. In some embodiments, the polynucleotide encodes a fusion protein that is at least 85% identical to the amino acid sequence of SEQ ID NO: 527. In some embodiments, the polynucleotide encodes a fusion protein that is at least 90% identical to the amino acid sequence of SEQ ID NO: 527. In some embodiments, the polynucleotide encodes a fusion protein that is at least 95% identical to the amino acid sequence of SEQ ID NO: 527. In some embodiments, the polynucleotide encodes a fusion protein that is at least 97% identical to the amino acid sequence of SEQ ID NO: 527. In some embodiments, the polynucleotide encodes a fusion protein that is at least 98% identical to the amino acid sequence of SEQ ID NO: 527. In some embodiments, the polynucleotide encodes a fusion protein that is at least 99% identical to the amino acid sequence of SEQ ID NO: 527. In some embodiments, the polynucleotide encodes a fusion protein that is at least 100% identical to the amino acid sequence of SEQ ID NO: 527. In some embodiments, the polynucleotide encodes a fusion protein that comprises 20, 19, 18, 17, 16, 15, 14, 13, 12, 11, 10, 9, 8, 7, 6, 5, 4, 3, 2, or 1 or fewer amino acid mismatches to the amino acid sequence of SEQ ID NO: 527. In some embodiments, the polynucleotide encodes a fusion protein that comprises 15 or fewer amino acid mismatches to the amino acid sequence of SEQ ID NO: 527. In some embodiments, the polynucleotide encodes a fusion protein that comprises 10 or fewer amino acid mismatches to the amino acid sequence of SEQ ID NO: 527. In some embodiments, the polynucleotide encodes a fusion protein that comprises 5 or fewer amino acid mismatches to the amino acid sequence of SEQ ID NO: 527. In some embodiments, the polynucleotide encodes a fusion protein that comprises 4 or fewer amino acid mismatches to the amino acid sequence of SEQ ID NO: 527. In some embodiments, the polynucleotide encodes a fusion protein that comprises 3 or fewer amino acid mismatches to the amino acid sequence of SEQ ID NO: 527. In some embodiments, the polynucleotide encodes a fusion protein that comprises 2 or fewer amino acid mismatches to the amino acid sequence of SEQ ID NO: 527. In some embodiments, the polynucleotide encodes a fusion protein that comprises 1 or fewer amino acid mismatches to the amino acid sequence of SEQ ID NO: 527.

In various embodiments, the polynucleotide encodes a fusion protein that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of SEQ ID NO: 528. In some embodiments, the polynucleotide encodes a fusion protein that is at least 85% identical to the amino acid sequence of SEQ ID NO: 528. In some embodiments, the polynucleotide encodes a fusion protein that is at least 90% identical to the amino acid sequence of SEQ ID NO: 528. In some embodiments, the polynucleotide encodes a fusion protein that is at least 95% identical to the amino acid sequence of SEQ ID NO: 528. In some embodiments, the polynucleotide encodes a fusion protein that is at least 97% identical to the amino acid sequence of SEQ ID NO: 528. In some embodiments, the polynucleotide encodes a fusion protein that is at least 98% identical to the amino acid sequence of SEQ ID NO: 528. In some embodiments, the polynucleotide encodes a fusion protein that is at least 99% identical to the amino acid sequence of SEQ ID NO: 528. In some embodiments, the polynucleotide encodes a fusion protein that is at least 100% identical to the amino acid sequence of SEQ ID NO: 528. In some embodiments, the polynucleotide encodes a fusion protein that comprises 20, 19, 18, 17, 16, 15, 14, 13, 12, 11, 10, 9, 8, 7, 6, 5, 4, 3, 2, or 1 or fewer amino acid mismatches to the amino acid sequence of SEQ ID NO: 528. In some embodiments, the polynucleotide encodes a fusion protein that comprises 15 or fewer amino acid mismatches to the amino acid sequence of SEQ ID NO: 528. In some embodiments, the polynucleotide encodes a fusion protein that comprises 10 or fewer amino acid mismatches to the amino acid sequence of SEQ ID NO: 528. In some embodiments, the polynucleotide encodes a fusion protein that comprises 5 or fewer amino acid mismatches to the amino acid sequence of SEQ ID NO: 528. In some embodiments, the polynucleotide encodes a fusion protein that comprises 4 or fewer amino acid mismatches to the amino acid sequence of SEQ ID NO: 528. In some embodiments, the polynucleotide encodes a fusion protein that comprises 3 or fewer amino acid mismatches to the amino acid sequence of SEQ ID NO: 528. In some embodiments, the polynucleotide encodes a fusion protein that comprises 2 or fewer amino acid mismatches to the amino acid sequence of SEQ ID NO: 528. In some embodiments, the polynucleotide encodes a fusion protein that comprises 1 or fewer amino acid mismatches to the amino acid sequence of SEQ ID NO: 528.

In various embodiments, the one or more polynucleotide encodes one or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of any one of SEQ ID NOs: 345-371, 373-377, 407-411, 422-423, and 430-435.

In various embodiments, the polynucleotide is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 524. In some embodiments, the polynucleotide is at least 85% identical to the nucleic acid sequence of SEQ ID NO: 524. In some embodiments, the polynucleotide is at least 90% identical to the nucleic acid sequence of SEQ ID NO: 524. In some embodiments, the polynucleotide is at least 95% identical to the nucleic acid sequence of SEQ ID NO: 524. In some embodiments, the polynucleotide is at least 97% identical to the nucleic acid sequence of SEQ ID NO: 524. In some embodiments, the polynucleotide is at least 98% identical to the nucleic acid sequence of SEQ ID NO: 524. In some embodiments, the polynucleotide is at least 99% identical to the nucleic acid sequence of SEQ ID NO: 524. In some embodiments, the polynucleotide is at least 100% identical to the nucleic acid sequence of SEQ ID NO: 524. In some embodiments, the polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 524. In some embodiments, the polynucleotide that comprises 15 or fewer nucleotide mismatches to the nucleic acid sequence of SEQ ID NO: 524. In some embodiments, the polynucleotide that comprises 10 or fewer nucleotide mismatches to the nucleic acid sequence of SEQ ID NO: 524. In some embodiments, the polynucleotide that comprises 5 or fewer nucleotide mismatches to the nucleic acid sequence of SEQ ID NO: 524. In some embodiments, the polynucleotide that comprises 4 or fewer nucleotide mismatches to the nucleic acid sequence of SEQ ID NO: 524. In some embodiments, the polynucleotide that comprises 3 or fewer nucleotide mismatches to the nucleic acid sequence of SEQ ID NO: 524. In some embodiments, the polynucleotide that comprises 2 or fewer nucleotide mismatches to the nucleic acid sequence of SEQ ID NO: 524. In some embodiments, the polynucleotide that comprises 1 or fewer nucleotide mismatches to the nucleic acid sequence of SEQ ID NO: 524. In some embodiments, the polynucleotide that comprises a codon-optimized nucleic acid sequence of SEQ ID NO: 524.

In various embodiments, the polynucleotide is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 525. In some embodiments, the polynucleotide is at least 85% identical to the nucleic acid sequence of SEQ ID NO: 525. In some embodiments, the polynucleotide is at least 90% identical to the nucleic acid sequence of SEQ ID NO: 525. In some embodiments, the polynucleotide is at least 95% identical to the nucleic acid sequence of SEQ ID NO: 525. In some embodiments, the polynucleotide is at least 97% identical to the nucleic acid sequence of SEQ ID NO: 525. In some embodiments, the polynucleotide is at least 98% identical to the nucleic acid sequence of SEQ ID NO: 525. In some embodiments, the polynucleotide is at least 99% identical to the nucleic acid sequence of SEQ ID NO: 525. In some embodiments, the polynucleotide is at least 100% identical to the nucleic acid sequence of SEQ ID NO: 525. In some embodiments, the polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 525. In some embodiments, the polynucleotide that comprises 15 or fewer nucleotide mismatches to the nucleic acid sequence of SEQ ID NO: 525. In some embodiments, the polynucleotide that comprises 10 or fewer nucleotide mismatches to the nucleic acid sequence of SEQ ID NO: 525. In some embodiments, the polynucleotide that comprises 5 or fewer nucleotide mismatches to the nucleic acid sequence of SEQ ID NO: 525. In some embodiments, the polynucleotide that comprises 4 or fewer nucleotide mismatches to the nucleic acid sequence of SEQ ID NO: 525. In some embodiments, the polynucleotide that comprises 3 or fewer nucleotide mismatches to the nucleic acid sequence of SEQ ID NO: 525. In some embodiments, the polynucleotide that comprises 2 or fewer nucleotide mismatches to the nucleic acid sequence of SEQ ID NO: 525. In some embodiments, the polynucleotide that comprises 1 or fewer nucleotide mismatches to the nucleic acid sequence of SEQ ID NO: 525. In some embodiments, the polynucleotide that comprises a codon-optimized nucleic acid sequence of SEQ ID NO: 525.

In various embodiments, the polynucleotide is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 526. In some embodiments, the polynucleotide is at least 85% identical to the nucleic acid sequence of SEQ ID NO: 526. In some embodiments, the polynucleotide is at least 90% identical to the nucleic acid sequence of SEQ ID NO: 526. In some embodiments, the polynucleotide is at least 95% identical to the nucleic acid sequence of SEQ ID NO: 526. In some embodiments, the polynucleotide is at least 97% identical to the nucleic acid sequence of SEQ ID NO: 526. In some embodiments, the polynucleotide is at least 98% identical to the nucleic acid sequence of SEQ ID NO: 526. In some embodiments, the polynucleotide is at least 99% identical to the nucleic acid sequence of SEQ ID NO: 526. In some embodiments, the polynucleotide is at least 100% identical to the nucleic acid sequence of SEQ ID NO: 526. In some embodiments, the polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 526. In some embodiments, the polynucleotide that comprises 15 or fewer nucleotide mismatches to the nucleic acid sequence of SEQ ID NO: 526. In some embodiments, the polynucleotide that comprises 10 or fewer nucleotide mismatches to the nucleic acid sequence of SEQ ID NO: 526. In some embodiments, the polynucleotide that comprises 5 or fewer nucleotide mismatches to the nucleic acid sequence of SEQ ID NO: 526. In some embodiments, the polynucleotide that comprises 4 or fewer nucleotide mismatches to the nucleic acid sequence of SEQ ID NO: 526. In some embodiments, the polynucleotide that comprises 3 or fewer nucleotide mismatches to the nucleic acid sequence of SEQ ID NO: 526. In some embodiments, the polynucleotide that comprises 2 or fewer nucleotide mismatches to the nucleic acid sequence of SEQ ID NO: 526. In some embodiments, the polynucleotide that comprises 1 or fewer nucleotide mismatches to the nucleic acid sequence of SEQ ID NO: 526. In some embodiments, the polynucleotide that comprises a codon-optimized nucleic acid sequence of SEQ ID NO: 526.

In various embodiments, the polynucleotide is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 522. In some embodiments, the polynucleotide is at least 85% identical to the nucleic acid sequence of SEQ ID NO: 522. In some embodiments, the polynucleotide is at least 90% identical to the nucleic acid sequence of SEQ ID NO: 522. In some embodiments, the polynucleotide is at least 95% identical to the nucleic acid sequence of SEQ ID NO: 522. In some embodiments, the polynucleotide is at least 97% identical to the nucleic acid sequence of SEQ ID NO: 522. In some embodiments, the polynucleotide is at least 98% identical to the nucleic acid sequence of SEQ ID NO: 522. In some embodiments, the polynucleotide is at least 99% identical to the nucleic acid sequence of SEQ ID NO: 522. In some embodiments, the polynucleotide is at least 100% identical to the nucleic acid sequence of SEQ ID NO: 522. In some embodiments, the polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 522. In some embodiments, the polynucleotide that comprises 15 or fewer nucleotide mismatches to the nucleic acid sequence of SEQ ID NO: 522. In some embodiments, the polynucleotide that comprises 10 or fewer nucleotide mismatches to the nucleic acid sequence of SEQ ID NO: 522. In some embodiments, the polynucleotide that comprises 5 or fewer nucleotide mismatches to the nucleic acid sequence of SEQ ID NO: 522. In some embodiments, the polynucleotide that comprises 4 or fewer nucleotide mismatches to the nucleic acid sequence of SEQ ID NO: 522. In some embodiments, the polynucleotide that comprises 3 or fewer nucleotide mismatches to the nucleic acid sequence of SEQ ID NO: 522. In some embodiments, the polynucleotide that comprises 2 or fewer nucleotide mismatches to the nucleic acid sequence of SEQ ID NO: 522. In some embodiments, the polynucleotide that comprises 1 or fewer nucleotide mismatches to the nucleic acid sequence of SEQ ID NO: 522. In some embodiments, the polynucleotide that comprises a codon-optimized nucleic acid sequence of SEQ ID NO: 522.

In various embodiments, the polynucleotide is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 523. In some embodiments, the polynucleotide is at least 85% identical to the nucleic acid sequence of SEQ ID NO: 523. In some embodiments, the polynucleotide is at least 90% identical to the nucleic acid sequence of SEQ ID NO: 523. In some embodiments, the polynucleotide is at least 95% identical to the nucleic acid sequence of SEQ ID NO: 523. In some embodiments, the polynucleotide is at least 97% identical to the nucleic acid sequence of SEQ ID NO: 523. In some embodiments, the polynucleotide is at least 98% identical to the nucleic acid sequence of SEQ ID NO: 523. In some embodiments, the polynucleotide is at least 99% identical to the nucleic acid sequence of SEQ ID NO: 523. In some embodiments, the polynucleotide is at least 100% identical to the nucleic acid sequence of SEQ ID NO: 523. In some embodiments, the polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 523. In some embodiments, the polynucleotide that comprises 15 or fewer nucleotide mismatches to the nucleic acid sequence of SEQ ID NO: 523. In some embodiments, the polynucleotide that comprises 10 or fewer nucleotide mismatches to the nucleic acid sequence of SEQ ID NO: 523. In some embodiments, the polynucleotide that comprises 5 or fewer nucleotide mismatches to the nucleic acid sequence of SEQ ID NO: 523. In some embodiments, the polynucleotide that comprises 4 or fewer nucleotide mismatches to the nucleic acid sequence of SEQ ID NO: 523. In some embodiments, the polynucleotide that comprises 3 or fewer nucleotide mismatches to the nucleic acid sequence of SEQ ID NO: 523. In some embodiments, the polynucleotide that comprises 2 or fewer nucleotide mismatches to the nucleic acid sequence of SEQ ID NO: 523. In some embodiments, the polynucleotide that comprises 1 or fewer nucleotide mismatches to the nucleic acid sequence of SEQ ID NO: 523. In some embodiments, the polynucleotide that comprises a codon-optimized nucleic acid sequence of SEQ ID NO: 523.

In various embodiments, the polynucleotide is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 520. In some embodiments, the polynucleotide is at least 85% identical to the nucleic acid sequence of SEQ ID NO: 520. In some embodiments, the polynucleotide is at least 90% identical to the nucleic acid sequence of SEQ ID NO: 520. In some embodiments, the polynucleotide is at least 95% identical to the nucleic acid sequence of SEQ ID NO: 520. In some embodiments, the polynucleotide is at least 97% identical to the nucleic acid sequence of SEQ ID NO: 520. In some embodiments, the polynucleotide is at least 98% identical to the nucleic acid sequence of SEQ ID NO: 520. In some embodiments, the polynucleotide is at least 99% identical to the nucleic acid sequence of SEQ ID NO: 520. In some embodiments, the polynucleotide is at least 100% identical to the nucleic acid sequence of SEQ ID NO: 520. In some embodiments, the polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 520. In some embodiments, the polynucleotide that comprises 15 or fewer nucleotide mismatches to the nucleic acid sequence of SEQ ID NO: 520. In some embodiments, the polynucleotide that comprises 10 or fewer nucleotide mismatches to the nucleic acid sequence of SEQ ID NO: 520. In some embodiments, the polynucleotide that comprises 5 or fewer nucleotide mismatches to the nucleic acid sequence of SEQ ID NO: 520. In some embodiments, the polynucleotide that comprises 4 or fewer nucleotide mismatches to the nucleic acid sequence of SEQ ID NO: 520. In some embodiments, the polynucleotide that comprises 3 or fewer nucleotide mismatches to the nucleic acid sequence of SEQ ID NO: 520. In some embodiments, the polynucleotide that comprises 2 or fewer nucleotide mismatches to the nucleic acid sequence of SEQ ID NO: 520. In some embodiments, the polynucleotide that comprises 1 or fewer nucleotide mismatches to the nucleic acid sequence of SEQ ID NO: 520. In some embodiments, the polynucleotide that comprises a codon-optimized nucleic acid sequence of SEQ ID NO: 520.

In various embodiments, the polynucleotide is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 521. In some embodiments, the polynucleotide is at least 85% identical to the nucleic acid sequence of SEQ ID NO: 521. In some embodiments, the polynucleotide is at least 90% identical to the nucleic acid sequence of SEQ ID NO: 521. In some embodiments, the polynucleotide is at least 95% identical to the nucleic acid sequence of SEQ ID NO: 521. In some embodiments, the polynucleotide is at least 97% identical to the nucleic acid sequence of SEQ ID NO: 521. In some embodiments, the polynucleotide is at least 98% identical to the nucleic acid sequence of SEQ ID NO: 521. In some embodiments, the polynucleotide is at least 99% identical to the nucleic acid sequence of SEQ ID NO: 521. In some embodiments, the polynucleotide is at least 100% identical to the nucleic acid sequence of SEQ ID NO: 521. In some embodiments, the polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 521. In some embodiments, the polynucleotide that comprises 15 or fewer nucleotide mismatches to the nucleic acid sequence of SEQ ID NO: 521. In some embodiments, the polynucleotide that comprises 10 or fewer nucleotide mismatches to the nucleic acid sequence of SEQ ID NO: 521. In some embodiments, the polynucleotide that comprises 5 or fewer nucleotide mismatches to the nucleic acid sequence of SEQ ID NO: 521. In some embodiments, the polynucleotide that comprises 4 or fewer nucleotide mismatches to the nucleic acid sequence of SEQ ID NO: 521. In some embodiments, the polynucleotide that comprises 3 or fewer nucleotide mismatches to the nucleic acid sequence of SEQ ID NO: 521. In some embodiments, the polynucleotide that comprises 2 or fewer nucleotide mismatches to the nucleic acid sequence of SEQ ID NO: 521. In some embodiments, the polynucleotide that comprises 1 or fewer nucleotide mismatches to the nucleic acid sequence of SEQ ID NO: 521. In some embodiments, the polynucleotide that comprises a codon-optimized nucleic acid sequence of SEQ ID NO: 521.

As appropriate, in certain embodiments, the 3′-end of the polynucleotide encoding the fusion polypeptides described herein comprises one or multiple tandem stop codons, e.g., two or more tandem TAG (“amber”), TAA (“ochre”) or TGA (“opal” or “umber”) stop codons. The multiple tandem stop codons can be the same or different.

Further provided are expression cassettes, comprising a polynucleotide encoding a fusion polypeptide, as described herein, operably linked to one or more regulatory sequences. In some embodiments, the polynucleotide is operably linked to and under the control of a constitutive promoter. In some embodiments, the promoter is selected from cytomegalovirus major immediate-early (CMV), the CMV enhancer fused to the chicken beta-actin promoter (CAG), human elongation factor-1a (HEF-1a), mouse cytomegalovirus (mouse CMV), Chinese hamster elongation factor-1a (CHEF-1a), and phosphoglycerate kinase (PGK).

### Expression Cassettes, Vectors, Viral Vectors, and Host Cells

Further provided are vectors comprising one or more polynucleotides encoding one or more of the fusion polypeptides, described herein, or an expression cassette comprising such polynucleotides. A vector can be of any type, for example, a recombinant vector such as an expression vector. Vectors include without limitation, plasmids, cosmids, bacterial artificial chromosomes (BAC) and yeast artificial chromosomes (YAC) and vectors derived from bacteriophages or plant or animal (including human) viruses. Vectors can comprise an origin of replication recognized by the proposed host cell and in the case of expression vectors, promoter and other regulatory regions recognized by the host cell. In additional embodiments, a vector comprises one or more polynucleotides encoding one or more fusion polypeptides of the disclosure operably linked to a promoter and optionally additional regulatory elements. Certain vectors are capable of autonomous replication in a host into which they are introduced (e.g., vectors having a bacterial origin of replication can replicate in bacteria). Other vectors can be integrated into the genome of a host upon introduction into the host, and thereby are replicated along with the host genome. Vectors include without limitation, those suitable for recombinant production of the fusion polypeptides disclosed herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule or polynucleotide of the present application. Certain vectors are capable of directing the expression of nucleic acids to which they are operatively linked. Such vectors are referred to herein as expression vectors.

The term “operably linked” refers to two or more nucleic acid sequence elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The choice of the vector is dependent on the recombinant procedures followed and the host used. Introduction of vectors into host cells can be effected by inter alia calcium phosphate transfection, DEAE-dextran-mediated transfection, lipofectamine transfection, electroporation, virus infection, or via administration to a subject, as described herein. Vectors may be autonomously replicating or may replicate together with the chromosome into which they have been integrated. In certain embodiments, the vectors contain one or more selection markers. The choice of the markers may depend on the host cells of choice. These include without limitation, kanamycin, neomycin, puromycin, hygromycin, zeocin, thymidine kinase gene from Herpes simplex virus (HSV-TK), and dihydrofolate reductase gene from mouse (dhfr). Vectors comprising one or more nucleic acid molecules encoding the fusion polypeptides described herein, operably linked to one or more nucleic acid molecules encoding proteins or peptides that can be used to isolate the fusion polypeptides (“purification tags”), are also covered by the disclosure. These proteins or peptides include without limitation, FLAG-tag (DYKDDDDKL; SEQ ID NO: 436), glutathione-S-transferase, maltose binding protein, metal-binding polyhistidine, green fluorescent protein, luciferase and beta-galactosidase.

In other embodiments, the vector that is used is pcDNA™3.1+ (ThermoFisher, MA).

In some embodiments, the vector is viral expression vector or viral vector. As used herein, a viral expression vector refers to a viral expression system or viral expression construct, whereas a viral vector refers to a viral particle. As appropriate, the viral expression vector or viral vector can be a DNA virus or a RNA virus, including a self-replicating RNA virus. Self-replicating RNA viruses include Alphaviruses, and are described, e.g., in Lundstrom, Molecules. (2018) 23 (12). pii: E3310 (PMID: 30551668); and Ljungberg, et al., Expert Rev Vaccines. (2015) 14 (2): 177-94). In various embodiments, the viral expression vector or viral vector is from a virus selected from the group consisting of adenovirus, adeno-associated virus, arenavirus, alphavirus, self-replicating alphavirus, poxvirus, cytomegalovirus, rhabdovirus, vesicular stomatitis virus, flavivirus, maraba virus and vaccinia virus. In some embodiments, the viral expression vector or viral vector is from a viral family selected from the group consisting of: Adenoviridae (e.g., Adenovirus, adeno-associated virus), Arenaviridae (e.g., lymphocytic choriomeningitis mammarenavirus, Cali mammarenavirus (a.k.a., Pichinde mammarenavirus), Herpesviridae (e.g., Cytomegalovirus, Herpesvirus, e.g., HSV-1), Parvoviridae (e.g., Parvovirus H1), Poxviridae (e.g. Vaccinia virus, e.g. modified vaccinia Ankara (MVA)), Paramyxoviridae (e.g. measles virus), Flaviviridae (e.g. Yellow fever virus), Reoviridae (e.g., Reovirus), Picornaviridae (e.g., Coxsackievirus, Seneca Valley Virus, Poliovirus), Paramyxoviridae (e.g., Measles virus, Newcastle disease virus (NDV)), Rhabdoviridae (e.g., Vesiculovirus, including Maraba vesiculovirus and Vesicular stomatitis virus (VSV)), Togaviridae (e.g., Alphavirus, e.g., self-replicating Alphavirus; Sindbis virus), Enteroviridae (e.g., Echovirus). Illustrative modified vaccinia viral expression vector or viral vectors of use for expressing the present fusion polypeptides are described, e.g., in WO 2019/134049.

In some embodiments, the viral expression vector or viral vector is an arenavirus vector selected from Lymphocytic choriomeningitis mammarenavirus (LCMV) (NCBI: txid11623), Cali mammarenavirus (a.k.a., Pichinde mammarenavirus or Pichinde arenavirus) (NCBI: txid2169993), Guanarito virus (GTOV) (NCBI: txid45219), Argentinian mammarenavirus (a.k.a., Junin virus (JUNV)) (NCBI: txid2169991), Lassa virus (LASV) (NCBI: txid11620), Lujo virus (LUJV) (NCBI: txid649188), Machupo virus (MACV) (NCBI: txid11628), Brazilian mammarenavirus (a.k.a., Sabia virus (SABV)) (NCBI: txid2169992), and Whitewater Arroyo virus (WWAV) (NCBI: txid46919). In some embodiments, the viral expression vector or viral vector is an arenavirus vector selected from Lymphocytic choriomeningitis mammarenavirus (LCMV) or Cali mammarenavirus (a.k.a., Pichinde mammarenavirus or Pichinde arenavirus). Illustrative arenavirus vectors that can be used as delivery and expression vehicles for the herein described fusion polypeptides are described, e.g., in WO 2009/083210; WO 2015/183895; WO 2016/075250; WO 2017/198726; and U.S. Pat. No. 9,943,585.

In some embodiments, the viral expression vector or viral vector is an adenovirus vector, e.g., from a human adenovirus or a simian adenovirus (e.g., a chimpanzee adenovirus, a gorilla adenovirus or a rhesus monkey adenovirus). In various embodiments, the adenovirus vector is selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (e.g. ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), ChAd147 (AdC147)), gorilla adenovirus (e.g. GC44, GC45, GC46) and rhesus adenovirus (e.g., RhAd51, RhAd52, RhAd53, RhAd54, RhAd55, RhAd56, RhAd57, RhAd58, RhAd59, RhAd60, RhAd61, RhAd62, RhAd63, RhAd64, RhAd65, RhAd66). Illustrative Chimpanzee, Gorilla and Rhesus monkey adenovirus vectors that can be used as delivery and expression vehicles for the herein described fusion polypeptides are described, e.g., in WO 2019/076880; WO 2019/076877; Andrabi et al., (2019) Cell Reports 27:2426-2441Guo, et al., Hum Vaccin Immunother. (2018) 14 (7): 1679-1685; Abbink, et al., J Virol. (2015) 89 (3): 1512-22; and Abbink, et al., J Virol. (2018) 92 (6). pii: e01924-17.

In various embodiments, the viral expression vector or viral vector is incapable of replication (i.e., replication defective or replication deficient), has reduced or diminished capacity for replication, e.g., in comparison to a wild-type viral vector (i.e., replication attenuated) or is replication competent.

In various embodiments, the expression cassette, expression vector, viral expression vector or viral vector is an adenoviral expression vector or viral vector comprising one or more polynucleotides that encode one or more fusion proteins comprising an amino acid sequence of any one of any one of SEQ ID NOs: 345-377, 407-411, 422-424, and 430-435, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 345-377, 407-411, 422-424, and 430-435.

In various embodiments, the expression cassette, expression vector, viral expression vector or viral vector comprises two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

In various embodiments, the expression cassette, expression vector, viral expression vector or viral vector comprises a polynucleotide encoding a fusion protein that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of SEQ ID NO: 527. In some embodiments, the expression cassette, expression vector, viral expression vector or viral vector comprises a polynucleotide encoding a fusion protein that is at least 85% identical to the amino acid sequence of SEQ ID NO: 527. In some embodiments, the expression cassette, expression vector, viral expression vector or viral vector comprises a polynucleotide encoding a fusion protein that is at least 90% identical to the amino acid sequence of SEQ ID NO: 527. In some embodiments, the expression cassette, expression vector, viral expression vector or viral vector comprises a polynucleotide encoding a fusion protein that is at least 95% identical to the amino acid sequence of SEQ ID NO: 527. In some embodiments, the expression cassette, expression vector, viral expression vector or viral vector comprises a polynucleotide encoding a fusion protein that is at least 97% identical to the amino acid sequence of SEQ ID NO: 527. In some embodiments, the expression cassette, expression vector, viral expression vector or viral vector comprises a polynucleotide encoding a fusion protein that is at least 98% identical to the amino acid sequence of SEQ ID NO: 527. In some embodiments, the expression cassette, expression vector, viral expression vector or viral vector comprises a polynucleotide encoding a fusion protein that is at least 99% identical to the amino acid sequence of SEQ ID NO: 527. In some embodiments, the expression cassette, expression vector, viral expression vector or viral vector comprises a polynucleotide encoding a fusion protein that is at least 100% identical to the amino acid sequence of SEQ ID NO: 527. In some embodiments, the expression cassette, expression vector, viral expression vector or viral vector comprises a polynucleotide encoding a fusion protein that comprises 20, 19, 18, 17, 16, 15, 14, 13, 12, 11, 10, 9, 8, 7, 6, 5, 4, 3, 2, or 1 or fewer amino acid mismatches to the amino acid sequence of SEQ ID NO: 527. In some embodiments, the expression cassette, expression vector, viral expression vector or viral vector comprises a polynucleotide encoding a fusion protein that comprises 15 or fewer amino acid mismatches to the amino acid sequence of SEQ ID NO: 527. In some embodiments, the expression cassette, expression vector, viral expression vector or viral vector comprises a polynucleotide encoding a fusion protein that comprises 10 or fewer amino acid mismatches to the amino acid sequence of SEQ ID NO: 527. In some embodiments, the expression cassette, expression vector, viral expression vector or viral vector comprises a polynucleotide encoding a fusion protein that comprises 5 or fewer amino acid mismatches to the amino acid sequence of SEQ ID NO: 527. In some embodiments, the expression cassette, expression vector, viral expression vector or viral vector comprises a polynucleotide encoding a fusion protein that comprises 4 or fewer amino acid mismatches to the amino acid sequence of SEQ ID NO: 527. In some embodiments, the expression cassette, expression vector, viral expression vector or viral vector comprises a polynucleotide encoding a fusion protein that comprises 3 or fewer amino acid mismatches to the amino acid sequence of SEQ ID NO: 527. In some embodiments, the expression cassette, expression vector, viral expression vector or viral vector comprises a polynucleotide encoding a fusion protein that comprises 2 or fewer amino acid mismatches to the amino acid sequence of SEQ ID NO: 527. In some embodiments, the expression cassette, expression vector, viral expression vector or viral vector comprises a polynucleotide encoding a fusion protein that comprises 1 or fewer amino acid mismatches to the amino acid sequence of SEQ ID NO: 527.

In various embodiments, the expression cassette, expression vector, viral expression vector or viral vector comprises a polynucleotide encoding a fusion protein that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of SEQ ID NO: 528. In some embodiments, the expression cassette, expression vector, viral expression vector or viral vector comprises a polynucleotide encoding a fusion protein that is at least 85% identical to the amino acid sequence of SEQ ID NO: 528. In some embodiments, the expression cassette, expression vector, viral expression vector or viral vector comprises a polynucleotide encoding a fusion protein that is at least 90% identical to the amino acid sequence of SEQ ID NO: 528. In some embodiments, the expression cassette, expression vector, viral expression vector or viral vector comprises a polynucleotide encoding a fusion protein that is at least 95% identical to the amino acid sequence of SEQ ID NO: 528. In some embodiments, the expression cassette, expression vector, viral expression vector or viral vector comprises a polynucleotide encoding a fusion protein that is at least 97% identical to the amino acid sequence of SEQ ID NO: 528. In some embodiments, the expression cassette, expression vector, viral expression vector or viral vector comprises a polynucleotide encoding a fusion protein that is at least 98% identical to the amino acid sequence of SEQ ID NO: 528. In some embodiments, the expression cassette, expression vector, viral expression vector or viral vector comprises a polynucleotide encoding a fusion protein that is at least 99% identical to the amino acid sequence of SEQ ID NO: 528. In some embodiments, the expression cassette, expression vector, viral expression vector or viral vector comprises a polynucleotide encoding a fusion protein that is at least 100% identical to the amino acid sequence of SEQ ID NO: 528. In some embodiments, the expression cassette, expression vector, viral expression vector or viral vector comprises a polynucleotide encoding a fusion protein that comprises 20, 19, 18, 17, 16, 15, 14, 13, 12, 11, 10, 9, 8, 7, 6, 5, 4, 3, 2, or 1 or fewer amino acid mismatches to the amino acid sequence of SEQ ID NO: 528. In some embodiments, the expression cassette, expression vector, viral expression vector or viral vector comprises a polynucleotide encoding a fusion protein that comprises 15 or fewer amino acid mismatches to the amino acid sequence of SEQ ID NO: 528. In some embodiments, the expression cassette, expression vector, viral expression vector or viral vector comprises a polynucleotide encoding a fusion protein that comprises 10 or fewer amino acid mismatches to the amino acid sequence of SEQ ID NO: 528. In some embodiments, the expression cassette, expression vector, viral expression vector or viral vector comprises a polynucleotide encoding a fusion protein that comprises 5 or fewer amino acid mismatches to the amino acid sequence of SEQ ID NO: 528. In some embodiments, the expression cassette, expression vector, viral expression vector or viral vector comprises a polynucleotide encoding a fusion protein that comprises 4 or fewer amino acid mismatches to the amino acid sequence of SEQ ID NO: 528. In some embodiments, the expression cassette, expression vector, viral expression vector or viral vector comprises a polynucleotide encoding a fusion protein that comprises 3 or fewer amino acid mismatches to the amino acid sequence of SEQ ID NO: 528. In some embodiments, the expression cassette, expression vector, viral expression vector or viral vector comprises a polynucleotide encoding a fusion protein that comprises 2 or fewer amino acid mismatches to the amino acid sequence of SEQ ID NO: 528. In some embodiments, the expression cassette, expression vector, viral expression vector or viral vector comprises a polynucleotide encoding a fusion protein that comprises 1 or fewer amino acid mismatches to the amino acid sequence of SEQ ID NO: 528.

In various embodiments, the expression cassette, expression vector, viral expression vector or viral vector comprises one or more polynucleotides encoding one or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of any one of SEQ ID NOs: 345-371, 373-377, 407-411, 422-423, and 430-435.

In various embodiments, the expression cassette, expression vector, viral expression vector or viral vector comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 524. In some embodiments, the expression cassette, expression vector, viral expression vector or viral vector comprises a polynucleotide that is at least 85% identical to the nucleic acid sequence of SEQ ID NO: 524. In some embodiments, the expression cassette, expression vector, viral expression vector or viral vector comprises a polynucleotide that is at least 90% identical to the nucleic acid sequence of SEQ ID NO: 524. In some embodiments, the expression cassette, expression vector, viral expression vector or viral vector comprises a polynucleotide that is at least 95% identical to the nucleic acid sequence of SEQ ID NO: 524. In some embodiments, the expression cassette, expression vector, viral expression vector or viral vector comprises a polynucleotide that is at least 97% identical to the nucleic acid sequence of SEQ ID NO: 524. In some embodiments, the expression cassette, expression vector, viral expression vector or viral vector comprises a polynucleotide that is at least 98% identical to the nucleic acid sequence of SEQ ID NO: 524. In some embodiments, the expression cassette, expression vector, viral expression vector or viral vector comprises a polynucleotide that is at least 99% identical to the nucleic acid sequence of SEQ ID NO: 524. In some embodiments, the expression cassette, expression vector, viral expression vector or viral vector comprises a polynucleotide that is at least 100% identical to the nucleic acid sequence of SEQ ID NO: 524. In some embodiments, the expression cassette, expression vector, viral expression vector or viral vector comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 524. In some embodiments, the expression cassette, expression vector, viral expression vector or viral vector comprises a polynucleotide that comprises 15 or fewer nucleotide mismatches to the nucleic acid sequence of SEQ ID NO: 524. In some embodiments, the expression cassette, expression vector, viral expression vector or viral vector comprises a polynucleotide that comprises 10 or fewer nucleotide mismatches to the nucleic acid sequence of SEQ ID NO: 524. In some embodiments, the expression cassette, expression vector, viral expression vector or viral vector comprises a polynucleotide that comprises 5 or fewer nucleotide mismatches to the nucleic acid sequence of SEQ ID NO: 524. In some embodiments, the expression cassette, expression vector, viral expression vector or viral vector comprises a polynucleotide that comprises 4 or fewer nucleotide mismatches to the nucleic acid sequence of SEQ ID NO: 524. In some embodiments, the expression cassette, expression vector, viral expression vector or viral vector comprises a polynucleotide that comprises 3 or fewer nucleotide mismatches to the nucleic acid sequence of SEQ ID NO: 524. In some embodiments, the expression cassette, expression vector, viral expression vector or viral vector comprises a polynucleotide that comprises 2 or fewer nucleotide mismatches to the nucleic acid sequence of SEQ ID NO: 524. In some embodiments, the expression cassette, expression vector, viral expression vector or viral vector comprises a polynucleotide that comprises 1 or fewer nucleotide mismatches to the nucleic acid sequence of SEQ ID NO: 524. In some embodiments, the expression cassette, expression vector, viral expression vector or viral vector comprises a polynucleotide that comprises a codon-optimized nucleic acid sequence of SEQ ID NO: 524.

In various embodiments, the expression cassette, expression vector, viral expression vector or viral vector comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 525. In some embodiments, the expression cassette, expression vector, viral expression vector or viral vector comprises a polynucleotide that is at least 85% identical to the nucleic acid sequence of SEQ ID NO: 525. In some embodiments, the expression cassette, expression vector, viral expression vector or viral vector comprises a polynucleotide that is at least 90% identical to the nucleic acid sequence of SEQ ID NO: 525. In some embodiments, the expression cassette, expression vector, viral expression vector or viral vector comprises a polynucleotide that is at least 95% identical to the nucleic acid sequence of SEQ ID NO: 525. In some embodiments, the expression cassette, expression vector, viral expression vector or viral vector comprises a polynucleotide that is at least 97% identical to the nucleic acid sequence of SEQ ID NO: 525. In some embodiments, the expression cassette, expression vector, viral expression vector or viral vector comprises a polynucleotide that is at least 98% identical to the nucleic acid sequence of SEQ ID NO: 525. In some embodiments, the expression cassette, expression vector, viral expression vector or viral vector comprises a polynucleotide that is at least 99% identical to the nucleic acid sequence of SEQ ID NO: 525. In some embodiments, the expression cassette, expression vector, viral expression vector or viral vector comprises a polynucleotide that is at least 100% identical to the nucleic acid sequence of SEQ ID NO: 525. In some embodiments, the expression cassette, expression vector, viral expression vector or viral vector comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 525. In some embodiments, the expression cassette, expression vector, viral expression vector or viral vector comprises a polynucleotide that comprises 15 or fewer nucleotide mismatches to the nucleic acid sequence of SEQ ID NO: 525. In some embodiments, the expression cassette, expression vector, viral expression vector or viral vector comprises a polynucleotide that comprises 10 or fewer nucleotide mismatches to the nucleic acid sequence of SEQ ID NO: 525. In some embodiments, the expression cassette, expression vector, viral expression vector or viral vector comprises a polynucleotide that comprises 5 or fewer nucleotide mismatches to the nucleic acid sequence of SEQ ID NO: 525. In some embodiments, the expression cassette, expression vector, viral expression vector or viral vector comprises a polynucleotide that comprises 4 or fewer nucleotide mismatches to the nucleic acid sequence of SEQ ID NO: 525. In some embodiments, the expression cassette, expression vector, viral expression vector or viral vector comprises a polynucleotide that comprises 3 or fewer nucleotide mismatches to the nucleic acid sequence of SEQ ID NO: 525. In some embodiments, the expression cassette, expression vector, viral expression vector or viral vector comprises a polynucleotide that comprises 2 or fewer nucleotide mismatches to the nucleic acid sequence of SEQ ID NO: 525. In some embodiments, the expression cassette, expression vector, viral expression vector or viral vector comprises a polynucleotide that comprises 1 or fewer nucleotide mismatches to the nucleic acid sequence of SEQ ID NO: 525. In some embodiments, the expression cassette, expression vector, viral expression vector or viral vector comprises a polynucleotide that comprises a codon-optimized nucleic acid sequence of SEQ ID NO: 525.

In various embodiments, the expression cassette, expression vector, viral expression vector or viral vector comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 526. In some embodiments, the expression cassette, expression vector, viral expression vector or viral vector comprises a polynucleotide that is at least 85% identical to the nucleic acid sequence of SEQ ID NO: 526. In some embodiments, the expression cassette, expression vector, viral expression vector or viral vector comprises a polynucleotide that is at least 90% identical to the nucleic acid sequence of SEQ ID NO: 526. In some embodiments, the expression cassette, expression vector, viral expression vector or viral vector comprises a polynucleotide that is at least 95% identical to the nucleic acid sequence of SEQ ID NO: 526. In some embodiments, the expression cassette, expression vector, viral expression vector or viral vector comprises a polynucleotide that is at least 97% identical to the nucleic acid sequence of SEQ ID NO: 526. In some embodiments, the expression cassette, expression vector, viral expression vector or viral vector comprises a polynucleotide that is at least 98% identical to the nucleic acid sequence of SEQ ID NO: 526. In some embodiments, the expression cassette, expression vector, viral expression vector or viral vector comprises a polynucleotide that is at least 99% identical to the nucleic acid sequence of SEQ ID NO: 526. In some embodiments, the expression cassette, expression vector, viral expression vector or viral vector comprises a polynucleotide that is at least 100% identical to the nucleic acid sequence of SEQ ID NO: 526. In some embodiments, the expression cassette, expression vector, viral expression vector or viral vector comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 526. In some embodiments, the expression cassette, expression vector, viral expression vector or viral vector comprises a polynucleotide that comprises 15 or fewer nucleotide mismatches to the nucleic acid sequence of SEQ ID NO: 526. In some embodiments, the expression cassette, expression vector, viral expression vector or viral vector comprises a polynucleotide that comprises 10 or fewer nucleotide mismatches to the nucleic acid sequence of SEQ ID NO: 526. In some embodiments, the expression cassette, expression vector, viral expression vector or viral vector comprises a polynucleotide that comprises 5 or fewer nucleotide mismatches to the nucleic acid sequence of SEQ ID NO: 526. In some embodiments, the expression cassette, expression vector, viral expression vector or viral vector comprises a polynucleotide that comprises 4 or fewer nucleotide mismatches to the nucleic acid sequence of SEQ ID NO: 526. In some embodiments, the expression cassette, expression vector, viral expression vector or viral vector comprises a polynucleotide that comprises 3 or fewer nucleotide mismatches to the nucleic acid sequence of SEQ ID NO: 526. In some embodiments, the expression cassette, expression vector, viral expression vector or viral vector comprises a polynucleotide that comprises 2 or fewer nucleotide mismatches to the nucleic acid sequence of SEQ ID NO: 526. In some embodiments, the expression cassette, expression vector, viral expression vector or viral vector comprises a polynucleotide that comprises 1 or fewer nucleotide mismatches to the nucleic acid sequence of SEQ ID NO: 526. In some embodiments, the expression cassette, expression vector, viral expression vector or viral vector comprises a polynucleotide that comprises a codon-optimized nucleic acid sequence of SEQ ID NO: 526.

In various embodiments, the expression cassette, expression vector, viral expression vector or viral vector comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 522. In some embodiments, the expression cassette, expression vector, viral expression vector or viral vector comprises a polynucleotide that is at least 85% identical to the nucleic acid sequence of SEQ ID NO: 522. In some embodiments, the expression cassette, expression vector, viral expression vector or viral vector comprises a polynucleotide that is at least 90% identical to the nucleic acid sequence of SEQ ID NO: 522. In some embodiments, the expression cassette, expression vector, viral expression vector or viral vector comprises a polynucleotide that is at least 95% identical to the nucleic acid sequence of SEQ ID NO: 522. In some embodiments, the expression cassette, expression vector, viral expression vector or viral vector comprises a polynucleotide that is at least 97% identical to the nucleic acid sequence of SEQ ID NO: 522. In some embodiments, the expression cassette, expression vector, viral expression vector or viral vector comprises a polynucleotide that is at least 98% identical to the nucleic acid sequence of SEQ ID NO: 522. In some embodiments, the expression cassette, expression vector, viral expression vector or viral vector comprises a polynucleotide that is at least 99% identical to the nucleic acid sequence of SEQ ID NO: 522. In some embodiments, the expression cassette, expression vector, viral expression vector or viral vector comprises a polynucleotide that is at least 100% identical to the nucleic acid sequence of SEQ ID NO: 522. In some embodiments, the expression cassette, expression vector, viral expression vector or viral vector comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 522. In some embodiments, the expression cassette, expression vector, viral expression vector or viral vector comprises a polynucleotide that comprises 15 or fewer nucleotide mismatches to the nucleic acid sequence of SEQ ID NO: 522. In some embodiments, the expression cassette, expression vector, viral expression vector or viral vector comprises a polynucleotide that comprises 10 or fewer nucleotide mismatches to the nucleic acid sequence of SEQ ID NO: 522. In some embodiments, the expression cassette, expression vector, viral expression vector or viral vector comprises a polynucleotide that comprises 5 or fewer nucleotide mismatches to the nucleic acid sequence of SEQ ID NO: 522. In some embodiments, the expression cassette, expression vector, viral expression vector or viral vector comprises a polynucleotide that comprises 4 or fewer nucleotide mismatches to the nucleic acid sequence of SEQ ID NO: 522. In some embodiments, the expression cassette, expression vector, viral expression vector or viral vector comprises a polynucleotide that comprises 3 or fewer nucleotide mismatches to the nucleic acid sequence of SEQ ID NO: 522. In some embodiments, the expression cassette, expression vector, viral expression vector or viral vector comprises a polynucleotide that comprises 2 or fewer nucleotide mismatches to the nucleic acid sequence of SEQ ID NO: 522. In some embodiments, the expression cassette, expression vector, viral expression vector or viral vector comprises a polynucleotide that comprises 1 or fewer nucleotide mismatches to the nucleic acid sequence of SEQ ID NO: 522. In some embodiments, the expression cassette, expression vector, viral expression vector or viral vector comprises a polynucleotide that comprises a codon-optimized nucleic acid sequence of SEQ ID NO: 522.

In various embodiments, the expression cassette, expression vector, viral expression vector or viral vector comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 523. In some embodiments, the expression cassette, expression vector, viral expression vector or viral vector comprises a polynucleotide that is at least 85% identical to the nucleic acid sequence of SEQ ID NO: 523. In some embodiments, the expression cassette, expression vector, viral expression vector or viral vector comprises a polynucleotide that is at least 90% identical to the nucleic acid sequence of SEQ ID NO: 523. In some embodiments, the expression cassette, expression vector, viral expression vector or viral vector comprises a polynucleotide that is at least 95% identical to the nucleic acid sequence of SEQ ID NO: 523. In some embodiments, the expression cassette, expression vector, viral expression vector or viral vector comprises a polynucleotide that is at least 97% identical to the nucleic acid sequence of SEQ ID NO: 523. In some embodiments, the expression cassette, expression vector, viral expression vector or viral vector comprises a polynucleotide that is at least 98% identical to the nucleic acid sequence of SEQ ID NO: 523. In some embodiments, the expression cassette, expression vector, viral expression vector or viral vector comprises a polynucleotide that is at least 99% identical to the nucleic acid sequence of SEQ ID NO: 523. In some embodiments, the expression cassette, expression vector, viral expression vector or viral vector comprises a polynucleotide that is at least 100% identical to the nucleic acid sequence of SEQ ID NO: 523. In some embodiments, the expression cassette, expression vector, viral expression vector or viral vector comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 523. In some embodiments, the expression cassette, expression vector, viral expression vector or viral vector comprises a polynucleotide that comprises 15 or fewer nucleotide mismatches to the nucleic acid sequence of SEQ ID NO: 523. In some embodiments, the expression cassette, expression vector, viral expression vector or viral vector comprises a polynucleotide that comprises 10 or fewer nucleotide mismatches to the nucleic acid sequence of SEQ ID NO: 523. In some embodiments, the expression cassette, expression vector, viral expression vector or viral vector comprises a polynucleotide that comprises 5 or fewer nucleotide mismatches to the nucleic acid sequence of SEQ ID NO: 523. In some embodiments, the expression cassette, expression vector, viral expression vector or viral vector comprises a polynucleotide that comprises 4 or fewer nucleotide mismatches to the nucleic acid sequence of SEQ ID NO: 523. In some embodiments, the expression cassette, expression vector, viral expression vector or viral vector comprises a polynucleotide that comprises 3 or fewer nucleotide mismatches to the nucleic acid sequence of SEQ ID NO: 523. In some embodiments, the expression cassette, expression vector, viral expression vector or viral vector comprises a polynucleotide that comprises 2 or fewer nucleotide mismatches to the nucleic acid sequence of SEQ ID NO: 523. In some embodiments, the expression cassette, expression vector, viral expression vector or viral vector comprises a polynucleotide that comprises 1 or fewer nucleotide mismatches to the nucleic acid sequence of SEQ ID NO: 523. In some embodiments, the expression cassette, expression vector, viral expression vector or viral vector comprises a polynucleotide that comprises a codon-optimized nucleic acid sequence of SEQ ID NO: 523.

In various embodiments, the expression cassette, expression vector, viral expression vector or viral vector comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 520. In some embodiments, the expression cassette, expression vector, viral expression vector or viral vector comprises a polynucleotide that is at least 85% identical to the nucleic acid sequence of SEQ ID NO: 520. In some embodiments, the expression cassette, expression vector, viral expression vector or viral vector comprises a polynucleotide that is at least 90% identical to the nucleic acid sequence of SEQ ID NO: 520. In some embodiments, the expression cassette, expression vector, viral expression vector or viral vector comprises a polynucleotide that is at least 95% identical to the nucleic acid sequence of SEQ ID NO: 520. In some embodiments, the expression cassette, expression vector, viral expression vector or viral vector comprises a polynucleotide that is at least 97% identical to the nucleic acid sequence of SEQ ID NO: 520. In some embodiments, the expression cassette, expression vector, viral expression vector or viral vector comprises a polynucleotide that is at least 98% identical to the nucleic acid sequence of SEQ ID NO: 520. In some embodiments, the expression cassette, expression vector, viral expression vector or viral vector comprises a polynucleotide that is at least 99% identical to the nucleic acid sequence of SEQ ID NO: 520. In some embodiments, the expression cassette, expression vector, viral expression vector or viral vector comprises a polynucleotide that is at least 100% identical to the nucleic acid sequence of SEQ ID NO: 520. In some embodiments, the expression cassette, expression vector, viral expression vector or viral vector comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 520. In some embodiments, the expression cassette, expression vector, viral expression vector or viral vector comprises a polynucleotide that comprises 15 or fewer nucleotide mismatches to the nucleic acid sequence of SEQ ID NO: 520. In some embodiments, the expression cassette, expression vector, viral expression vector or viral vector comprises a polynucleotide that comprises 10 or fewer nucleotide mismatches to the nucleic acid sequence of SEQ ID NO: 520. In some embodiments, the expression cassette, expression vector, viral expression vector or viral vector comprises a polynucleotide that comprises 5 or fewer nucleotide mismatches to the nucleic acid sequence of SEQ ID NO: 520. In some embodiments, the expression cassette, expression vector, viral expression vector or viral vector comprises a polynucleotide that comprises 4 or fewer nucleotide mismatches to the nucleic acid sequence of SEQ ID NO: 520. In some embodiments, the expression cassette, expression vector, viral expression vector or viral vector comprises a polynucleotide that comprises 3 or fewer nucleotide mismatches to the nucleic acid sequence of SEQ ID NO: 520. In some embodiments, the expression cassette, expression vector, viral expression vector or viral vector comprises a polynucleotide that comprises 2 or fewer nucleotide mismatches to the nucleic acid sequence of SEQ ID NO: 520. In some embodiments, the expression cassette, expression vector, viral expression vector or viral vector comprises a polynucleotide that comprises 1 or fewer nucleotide mismatches to the nucleic acid sequence of SEQ ID NO: 520. In some embodiments, the expression cassette, expression vector, viral expression vector or viral vector comprises a polynucleotide that comprises a codon-optimized nucleic acid sequence of SEQ ID NO: 520.

In various embodiments, the expression cassette, expression vector, viral expression vector or viral vector comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 521. In some embodiments, the expression cassette, expression vector, viral expression vector or viral vector comprises a polynucleotide that is at least 85% identical to the nucleic acid sequence of SEQ ID NO: 521. In some embodiments, the expression cassette, expression vector, viral expression vector or viral vector comprises a polynucleotide that is at least 90% identical to the nucleic acid sequence of SEQ ID NO: 521. In some embodiments, the expression cassette, expression vector, viral expression vector or viral vector comprises a polynucleotide that is at least 95% identical to the nucleic acid sequence of SEQ ID NO: 521. In some embodiments, the expression cassette, expression vector, viral expression vector or viral vector comprises a polynucleotide that is at least 97% identical to the nucleic acid sequence of SEQ ID NO: 521. In some embodiments, the expression cassette, expression vector, viral expression vector or viral vector comprises a polynucleotide that is at least 98% identical to the nucleic acid sequence of SEQ ID NO: 521. In some embodiments, the expression cassette, expression vector, viral expression vector or viral vector comprises a polynucleotide that is at least 99% identical to the nucleic acid sequence of SEQ ID NO: 521. In some embodiments, the expression cassette, expression vector, viral expression vector or viral vector comprises a polynucleotide that is at least 100% identical to the nucleic acid sequence of SEQ ID NO: 521. In some embodiments, the expression cassette, expression vector, viral expression vector or viral vector comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 521. In some embodiments, the expression cassette, expression vector, viral expression vector or viral vector comprises a polynucleotide that comprises 15 or fewer nucleotide mismatches to the nucleic acid sequence of SEQ ID NO: 521. In some embodiments, the expression cassette, expression vector, viral expression vector or viral vector comprises a polynucleotide that comprises 10 or fewer nucleotide mismatches to the nucleic acid sequence of SEQ ID NO: 521. In some embodiments, the expression cassette, expression vector, viral expression vector or viral vector comprises a polynucleotide that comprises 5 or fewer nucleotide mismatches to the nucleic acid sequence of SEQ ID NO: 521. In some embodiments, the expression cassette, expression vector, viral expression vector or viral vector comprises a polynucleotide that comprises 4 or fewer nucleotide mismatches to the nucleic acid sequence of SEQ ID NO: 521. In some embodiments, the expression cassette, expression vector, viral expression vector or viral vector comprises a polynucleotide that comprises 3 or fewer nucleotide mismatches to the nucleic acid sequence of SEQ ID NO: 521. In some embodiments, the expression cassette, expression vector, viral expression vector or viral vector comprises a polynucleotide that comprises 2 or fewer nucleotide mismatches to the nucleic acid sequence of SEQ ID NO: 521. In some embodiments, the expression cassette, expression vector, viral expression vector or viral vector comprises a polynucleotide that comprises 1 or fewer nucleotide mismatches to the nucleic acid sequence of SEQ ID NO: 521. In some embodiments, the expression cassette, expression vector, viral expression vector or viral vector comprises a polynucleotide that comprises a codon-optimized nucleic acid sequence of SEQ ID NO: 521.

FIG. 14A shows a linear schematic of a viral vector comprising a promoter sequence, an immunogen sequence (e.g., a sequence encoding an immunogenic polypeptide, such as an HIV immunogenic polypeptide or fusion polypeptide), and a polyA sequence. Exemplary viral vector sequences, such as SEQ ID NOs: 520, 521, 534, and 535, and the nucleotides corresponding to the promoter, immunogen, and polyA sequence are also shown in FIG. 14A. In some embodiments, the CMV promoter sequence, corresponding to nucleotides 594 to 1195 of SEQ ID NOs: 520, 521, 534, and 535, is replaced with another promoter sequence. In other words, in some embodiments, the CMV promoter of any of the viral vectors disclosed herein, such as SEQ ID NOs: 520, 521, 534, or 535, is replaced with any of the promoters disclosed herein, such as an SV40 promoter. In some embodiments, the immunogen sequence, corresponding to nucleotides 1256 to 4351 of SEQ ID NO: 520, or nucleotides 1256 to 4348 of SEQ ID NO: 521, or nucleotides 1256 to 2779 of SEQ ID NO: 534, or nucleotides 1256 to 3358 of SEQ ID NO: 535, is replaced with any of the immunogen sequences disclosed herein. In some embodiments, the immunogen sequence disclosed herein comprises any of the HIV immunogenic polypeptides or fusion proteins disclosed herein. In some embodiments, the polyA, corresponding to nucleotides 4596 to 4619 of SEQ ID NO: 520, or nucleotides 4393 to 4616 of SEQ ID NO: 521, or nucleotides 2824 to 3047 of SEQ ID NO: 534, or nucleotides 3406 to 3629 of SEQ ID NO: 535, is replaced with another polyA sequence.

In some embodiments, the viral vector of SEQ ID NO: 520 is modified such that the immunogen sequence, corresponding to nucleotides 1256 to 4351 of SEQ ID NO: 520, is replaced with a nucleotide sequence that is at least 90%, 91%, 92%, 93%, 94%, 95%, 96%, 97%, 98% or at least 99% identical to the nucleotide sequence of SEQ ID NO: 525 or 526, wherein percent identity is across the full length of SEQ ID NO: 525 or 526. In some embodiments, the viral vector of SEQ ID NO: 520 is modified such that the immunogen sequence, corresponding to nucleotides 1256 to 4351 of SEQ ID NO: 520, is replaced with a nucleotide sequence that encodes an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528.

In some embodiments, the viral vector of SEQ ID NO: 521 is modified such that the immunogen sequence, corresponding to nucleotides 1256 to 4348 of SEQ ID NO: 521, is replaced with a nucleotide sequence that is at least 90%, 91%, 92%, 93%, 94%, 95%, 96%, 97%, 98% or at least 99% identical to the nucleotide sequence of SEQ ID NO: 524, wherein percent identity is across the full length of SEQ ID NO: 524. In some embodiments, the viral vector of SEQ ID NO: 521 is modified such that the immunogen sequence, corresponding to nucleotides 1256 to 4348 of SEQ ID NO: 521, is replaced with a nucleotide sequence that encodes an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528.

In some embodiments, the viral vector of SEQ ID NO: 534 is modified such that the immunogen sequence, corresponding to nucleotides 1256 to 2779 of SEQ ID NO: 534, is replaced with a nucleotide sequence that is at least 90%, 91%, 92%, 93%, 94%, 95%, 96%, 97%, 98% or at least 99% identical to the nucleotide sequence of SEQ ID NO: 524, 525, or 526, wherein percent identity is across the full length of SEQ ID NO: 524, 525, or 526. In some embodiments, the viral vector of SEQ ID NO: 534 is modified such that the immunogen sequence, corresponding to nucleotides 1256 to 2779 of SEQ ID NO: 534, is replaced with a nucleotide sequence that encodes an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528.

In some embodiments, the viral vector of SEQ ID NO: 535 is modified such that the immunogen sequence, corresponding to nucleotides 1256 to 3358 of SEQ ID NO: 535, is replaced with a nucleotide sequence that is at least 90%, 91%, 92%, 93%, 94%, 95%, 96%, 97%, 98% or at least 99% identical to the nucleotide sequence of SEQ ID NO: 524, 525, or 526, wherein percent identity is across the full length of SEQ ID NO: 524, 525, or 526. In some embodiments, the viral vector of SEQ ID NO: 535 is modified such that the immunogen sequence, corresponding to nucleotides 1256 to 3358 of SEQ ID NO: 535, is replaced with a nucleotide sequence that encodes an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528.

FIG. 14B shows a linear schematic of a viral vector comprising a promoter sequence, an immunogen sequence (e.g., a sequence encoding an immunogenic polypeptide, such as an HIV immunogenic polypeptide or fusion polypeptide), and a polyA sequence. Exemplary viral vector sequences, such as SEQ ID NOs: 522, 523, and 537, and the nucleotides corresponding to the promoter, immunogen, and polyA sequence are also shown in FIG. 14B. In some embodiments, the 26S subgenomic promoter sequence, corresponding to nucleotides 7513 to 7536 of SEQ ID NOs: 522, 523, and 537, is replaced with another subgenomic promoter sequence. In other words, in some embodiments, the 26S subgenomic promoter of any of the viral vectors disclosed herein, such as SEQ ID NOs: 522, 523, and 537, is replaced with any of the promoters disclosed herein, such as a subgenomic promoter derived from alphavirus. In some embodiments, the immunogen sequence, corresponding to nucleotides 7571 to 10666 of SEQ ID NO: 522, or nucleotides 7571 to 10663 of SEQ ID NO: 523, or nucleotides 7571 to 9673 of SEQ ID NO: 538, is replaced with any of the immunogen sequences disclosed herein. In some embodiments, the immunogen sequence disclosed herein comprises any of the HIV immunogenic polypeptides or fusion proteins disclosed herein. In some embodiments, the polyA, corresponding to nucleotides 10953 to 11072 of SEQ ID NO: 522, or nucleotides 10950 to 11069 of SEQ ID NO: 523, or nucleotides 9960 to 10039 of SEQ ID NO: 537, is replaced with another polyA sequence.

In some embodiments, the viral vector of SEQ ID NO: 522 is modified such that the immunogen sequence, corresponding to nucleotides 7571 to 10666 of SEQ ID NO: 522, is replaced with a nucleotide sequence that is at least 90%, 91%, 92%, 93%, 94%, 95%, 96%, 97%, 98% or at least 99% identical to the nucleotide sequence of SEQ ID NO: 525 or 526, wherein percent identity is across the full length of SEQ ID NO: 525 or 526. In some embodiments, the viral vector of SEQ ID NO: 522 is modified such that the immunogen sequence, corresponding to nucleotides 7571 to 10666 of SEQ ID NO: 522, is replaced with a nucleotide sequence that encodes an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528.

In some embodiments, the viral vector of SEQ ID NO: 523 is modified such that the immunogen sequence, corresponding to nucleotides 7571 to 10663 of SEQ ID NO: 523, is replaced with a nucleotide sequence that is at least 90%, 91%, 92%, 93%, 94%, 95%, 96%, 97%, 98% or at least 99% identical to the nucleotide sequence of SEQ ID NO: 524, wherein percent identity is across the full length of SEQ ID NO: 524. In some embodiments, the viral vector of SEQ ID NO: 523 is modified such that the immunogen sequence, corresponding to nucleotides 7571 to 10663 of SEQ ID NO: 523, is replaced with a nucleotide sequence that encodes an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOS: 345-371, 373-377, 407-411, 422-423, 430-435, 527, and 528.

In some embodiments, the viral vector of SEQ ID NO: 537 is modified such that the immunogen sequence, corresponding to nucleotides 7571 to 9673 of SEQ ID NO: 537, is replaced with a nucleotide sequence that is at least 90%, 91%, 92%, 93%, 94%, 95%, 96%, 97%, 98% or at least 99% identical to the nucleotide sequence of SEQ ID NO: 524, 525, or 526, wherein percent identity is across the full length of SEQ ID NO: 524, 525, or 526. In some embodiments, the viral vector of SEQ ID NO: 537 is modified such that the immunogen sequence, corresponding to nucleotides 7571 to 9673 of SEQ ID NO: 537, is replaced with a nucleotide sequence that encodes an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528.

In some embodiments, the expression vector, viral expression vector, or viral vector further comprises a polynucleotide encoding a cytokine or functional variant thereof, or a non-coding immunostimulatory polynucleotide. In some embodiments, the expression vector, viral expression vector, or viral vector further comprises a polynucleotide encoding a cytokine selected from the group consisting of IL-2, IL-7, IL-12, IL-15, IL-18, IL-21, IFN-α, IFN-γ, colony stimulating factor 2 (CSF2; a.k.a., GM-CSF), fms related receptor tyrosine kinase 3 ligand (FLT3LG), and combinations and functional variants thereof. Co-expression and/or co-administration of a cytokine with a vaccine is described, e.g., by Elizaga, et al. (2018) PLoS One 13 (9): e0202753 (IL-12); Buchbinder, et al., (2017) PLoS One 12 (7): e0179597 (GM-CSF); Abaitua, et al., Virus Res (2006) 116 (1-2): 11-20 (IL12+IFN-γ); Oudard, et al., Cancer Immunol Immunother (2011) February; 60 (2): 261-71 (IL-2+IFN-α). In some embodiments, the vector (e.g., the expression vector, viral expression vector, or viral vector) further comprises a non-coding immunostimulatory polynucleotide selected from a pathogen-activated molecular pattern (PAMP), a cytosine-phosphate-guanosine (CpG) oligodeoxynucleotide, and an immunostimulatory RNA (isRNA). Illustrative isRNA include CV8102 (CureVac) and others, described in e.g., WO2016170176.

Further provided are host cells comprising one or more polynucleotides encoding one or more of the fusion polypeptides or one or more vectors expressing the fusion polypeptides, as described herein. Any of a variety of host cells can be used. In one embodiment, a host cell is a prokaryotic cell, for example, E. coli. In another embodiment, a host cell is a eukaryotic cell, for example, a yeast cell, a plant cell, an insect cell, a mammalian cell, such as a Chinese Hamster Ovary (CHO)-based or CHO-origin cell line (e.g., CHO-S, CHO DG44, ExpiCHO™, CHOZN® ZFN-modified GS−/− CHO cell line, CHO-K1, CHO-K1a), COS cells, BHK cells, NSO cells or Bowes melanoma cells. Examples of human host cells are, inter alia, HeLa, 911, AT1080, A549 and HEK293 (e.g., HEK293E, HEK293F, HEK293H, HEK293T, Expi293™). In addition, the fusion polypeptides can be expressed in a yeast cell such as Pichia (see, e.g., Powers et al., J Immunol Methods. 251:123-35 (2001)), Hanseula, or Saccharomyces.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with one or more polynucleotides encoding one or more fusion polypeptides, as described herein. As appropriate, the host cells can be infected with one or more vectors expressing one or more fusion polypeptides, as described herein. In some embodiments, the host cells are capable of being infected with and propagating one or more replication attenuated or replication competent vectors expressing one or more fusion polypeptides, as described herein. Illustrative cells useful for infecting with and/or propagating viral vectors include without limitation BHK-21, A549, Vero and HEK293 (e.g., HEK293E, HEK293F, HEK293H, HEK293T, Expi293™) cells. In certain embodiments, the host cells express the Coxsackievirus and adenovirus receptor (CAR), e.g., MDCK, Caco-2 or Calu-3 host cells. In certain embodiments, the polynucleotides integrate into the genome of the host cell.

### Lipid Nanoparticles, Polymeric Nanoparticles, and Nanoemulsions

In certain embodiments, any of the polynucleotides or vectors disclosed herein are formulated into lipid nanoparticles. For example, in some embodiments where the fusion polypeptides are expressed from self-replicating or self-amplifying RNA molecules, the self-replicating or self-amplifying RNA can be formulated into lipoplexes, such as lipid nanoparticles (LNPs), polymeric nanoparticles (PNPs), and nanoemulsions. As used herein, a “lipoplex” refers to cationic liposomes that are nonviral (synthetic) lipid carriers of DNA.

As used herein, the term “lipid nanoparticle” refers to one or more spherical nanoparticles with an average diameter of between about 10 to about 1000 nanometers, and which comprise a solid lipid core matrix that can solubilize lipophilic molecules. In certain embodiments, the lipid core is stabilized by surfactants (e.g., emulsifiers), and can comprise one or more of triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate), including combinations thereof. Lipid nanoparticles are described, for example, in Petrilli et al., Curr Pharm Biotechnol. 15:847-55, 2014; and U.S. Pat. Nos. 6,217,912; 6,881,421; 7,402,573; 7,404,969; 7,550,441; 7,727,969; 8,003,621; 8,691,750; 8,871,509; 9,017,726; 9,173,853; 9,220,779; 9,227,917; and 9,278,130, each of which is incorporated by reference in its entirety. In one embodiment, a self-replicating or self-amplifying RNA molecule encoding one or more of the fusion polypeptides described herein is formulated or condensed into polyethylenimine (PEI)-polyplex delivery vehicles, e.g., as described in Démoulins, et al., Nanomedicine. (2016) April; 12 (3): 711-722 and Démoulins, et al., J Control Release. (2017) November 28; 266:256-271, which can be nanoparticulate. In some embodiments, the LNP comprises or is synthesized from an ionizable lipid, phospholipid, cholesterol, PEGylated lipid, or any combination thereof. In some embodiments, the LNP comprises or is synthesized from cationic lipid 1,2-dioleoyl-3-timethylammonium-propane (DOTAP).

In some embodiments, the PNP comprises or is synthesized from non-degradable polymers, degradable polymers, natural materials, synthetic materials, monomers, dendrimers, or any combination thereof.

In some embodiments, the PNP comprises or is synthesized from poly(ethylene glycol) (PEG), poly(dimethylsiloxane) (PDMS), polyethyleneimine (PEI), poly(amidoamine) (PAMAM), poly(dimethylaminoethyl) acrylate (pDMAEA), orthenine-derived dendrimers, or any combination thereof. In some embodiments, the PNP comprises or is synthesized from mannosylated-PEI polyplexes. In some embodiments, the PNP comprises or is synthesized from a bioreducible, linear, cationic polymer (pABOL).

In some embodiments, the nanoemulsion is a water-in-oil emulsion. In some embodiments, the nanoemulsion comprises squalene, sorbitan trioleate, polysorbate 80, DOTAP, or any combination thereof.

In various embodiments, the SAM, LNP, PNP, or nanoemulsion comprises one or more polynucleotides that encode one or more fusion proteins comprising an amino acid sequence of any one of any one of SEQ ID NOs: 345-377, 407-411, 422-424, and 430-435, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 345-377, 407-411, 422-424, and 430-435.

In various embodiments, the SAM, LNP, PNP, or nanoemulsion comprises two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

In various embodiments, the SAM, LNP, PNP, or nanoemulsion comprises a polynucleotide encoding a fusion protein that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of SEQ ID NO: 527. In some embodiments, the SAM, LNP, PNP, or nanoemulsion comprises a polynucleotide encoding a fusion protein that is at least 85% identical to the amino acid sequence of SEQ ID NO: 527. In some embodiments, the SAM, LNP, PNP, or nanoemulsion comprises a polynucleotide encoding a fusion protein that is at least 90% identical to the amino acid sequence of SEQ ID NO: 527. In some embodiments, the SAM, LNP, PNP, or nanoemulsion comprises a polynucleotide encoding a fusion protein that is at least 95% identical to the amino acid sequence of SEQ ID NO: 527. In some embodiments, the SAM, LNP, PNP, or nanoemulsion comprises a polynucleotide encoding a fusion protein that is at least 97% identical to the amino acid sequence of SEQ ID NO: 527. In some embodiments, the SAM, LNP, PNP, or nanoemulsion comprises a polynucleotide encoding a fusion protein that is at least 98% identical to the amino acid sequence of SEQ ID NO: 527. In some embodiments, the SAM, LNP, PNP, or nanoemulsion comprises a polynucleotide encoding a fusion protein that is at least 99% identical to the amino acid sequence of SEQ ID NO: 527. In some embodiments, the SAM, LNP, PNP, or nanoemulsion comprises a polynucleotide encoding a fusion protein that is at least 100% identical to the amino acid sequence of SEQ ID NO: 527. In some embodiments, the SAM, LNP, PNP, or nanoemulsion comprises a polynucleotide encoding a fusion protein that comprises 20, 19, 18, 17, 16, 15, 14, 13, 12, 11, 10, 9, 8, 7, 6, 5, 4, 3, 2, or 1 or fewer amino acid mismatches to the amino acid sequence of SEQ ID NO: 527. In some embodiments, the SAM, LNP, PNP, or nanoemulsion comprises a polynucleotide encoding a fusion protein that comprises 15 or fewer amino acid mismatches to the amino acid sequence of SEQ ID NO: 527. In some embodiments, the SAM, LNP, PNP, or nanoemulsion comprises a polynucleotide encoding a fusion protein that comprises 10 or fewer amino acid mismatches to the amino acid sequence of SEQ ID NO: 527. In some embodiments, the SAM, LNP, PNP, or nanoemulsion comprises a polynucleotide encoding a fusion protein that comprises 5 or fewer amino acid mismatches to the amino acid sequence of SEQ ID NO: 527. In some embodiments, the SAM, LNP, PNP, or nanoemulsion comprises a polynucleotide encoding a fusion protein that comprises 4 or fewer amino acid mismatches to the amino acid sequence of SEQ ID NO: 527. In some embodiments, the SAM, LNP, PNP, or nanoemulsion comprises a polynucleotide encoding a fusion protein that comprises 3 or fewer amino acid mismatches to the amino acid sequence of SEQ ID NO: 527. In some embodiments, the SAM, LNP, PNP, or nanoemulsion comprises a polynucleotide encoding a fusion protein that comprises 2 or fewer amino acid mismatches to the amino acid sequence of SEQ ID NO: 527. In some embodiments, the SAM, LNP, PNP, or nanoemulsion comprises a polynucleotide encoding a fusion protein that comprises 1 or fewer amino acid mismatches to the amino acid sequence of SEQ ID NO: 527.

In various embodiments, the SAM, LNP, PNP, or nanoemulsion comprises a polynucleotide encoding a fusion protein that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of SEQ ID NO: 528. In some embodiments, the SAM, LNP, PNP, or nanoemulsion comprises a polynucleotide encoding a fusion protein that is at least 85% identical to the amino acid sequence of SEQ ID NO: 528. In some embodiments, the SAM, LNP, PNP, or nanoemulsion comprises a polynucleotide encoding a fusion protein that is at least 90% identical to the amino acid sequence of SEQ ID NO: 528. In some embodiments, the SAM, LNP, PNP, or nanoemulsion comprises a polynucleotide encoding a fusion protein that is at least 95% identical to the amino acid sequence of SEQ ID NO: 528. In some embodiments, the SAM, LNP, PNP, or nanoemulsion comprises a polynucleotide encoding a fusion protein that is at least 97% identical to the amino acid sequence of SEQ ID NO: 528. In some embodiments, the SAM, LNP, PNP, or nanoemulsion comprises a polynucleotide encoding a fusion protein that is at least 98% identical to the amino acid sequence of SEQ ID NO: 528. In some embodiments, the SAM, LNP, PNP, or nanoemulsion comprises a polynucleotide encoding a fusion protein that is at least 99% identical to the amino acid sequence of SEQ ID NO: 528. In some embodiments, the SAM, LNP, PNP, or nanoemulsion comprises a polynucleotide encoding a fusion protein that is at least 100% identical to the amino acid sequence of SEQ ID NO: 528. In some embodiments, the SAM, LNP, PNP, or nanoemulsion comprises a polynucleotide encoding a fusion protein that comprises 20, 19, 18, 17, 16, 15, 14, 13, 12, 11, 10, 9, 8, 7, 6, 5, 4, 3, 2, or 1 or fewer amino acid mismatches to the amino acid sequence of SEQ ID NO: 528. In some embodiments, the SAM, LNP, PNP, or nanoemulsion comprises a polynucleotide encoding a fusion protein that comprises 15 or fewer amino acid mismatches to the amino acid sequence of SEQ ID NO: 528. In some embodiments, the SAM, LNP, PNP, or nanoemulsion comprises a polynucleotide encoding a fusion protein that comprises 10 or fewer amino acid mismatches to the amino acid sequence of SEQ ID NO: 528. In some embodiments, the SAM, LNP, PNP, or nanoemulsion comprises a polynucleotide encoding a fusion protein that comprises 5 or fewer amino acid mismatches to the amino acid sequence of SEQ ID NO: 528. In some embodiments, the SAM, LNP, PNP, or nanoemulsion comprises a polynucleotide encoding a fusion protein that comprises 4 or fewer amino acid mismatches to the amino acid sequence of SEQ ID NO: 528. In some embodiments, the SAM, LNP, PNP, or nanoemulsion comprises a polynucleotide encoding a fusion protein that comprises 3 or fewer amino acid mismatches to the amino acid sequence of SEQ ID NO: 528. In some embodiments, the SAM, LNP, PNP, or nanoemulsion comprises a polynucleotide encoding a fusion protein that comprises 2 or fewer amino acid mismatches to the amino acid sequence of SEQ ID NO: 528. In some embodiments, the SAM, LNP, PNP, or nanoemulsion comprises a polynucleotide encoding a fusion protein that comprises 1 or fewer amino acid mismatches to the amino acid sequence of SEQ ID NO: 528.

In various embodiments, the SAM, LNP, PNP, or nanoemulsion comprises one or more polynucleotides encoding one or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of any one of SEQ ID NOs: 345-371, 373-377, 407-411, 422-423, and 430-435.

In various embodiments, the SAM, LNP, PNP, or nanoemulsion comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 524. In some embodiments, the SAM, LNP, PNP, or nanoemulsion comprises a polynucleotide that is at least 85% identical to the nucleic acid sequence of SEQ ID NO: 524. In some embodiments, the SAM, LNP, PNP, or nanoemulsion comprises a polynucleotide that is at least 90% identical to the nucleic acid sequence of SEQ ID NO: 524. In some embodiments, the SAM, LNP, PNP, or nanoemulsion comprises a polynucleotide that is at least 95% identical to the nucleic acid sequence of SEQ ID NO: 524. In some embodiments, the SAM, LNP, PNP, or nanoemulsion comprises a polynucleotide that is at least 97% identical to the nucleic acid sequence of SEQ ID NO: 524. In some embodiments, the SAM, LNP, PNP, or nanoemulsion comprises a polynucleotide that is at least 98% identical to the nucleic acid sequence of SEQ ID NO: 524. In some embodiments, the SAM, LNP, PNP, or nanoemulsion comprises a polynucleotide that is at least 99% identical to the nucleic acid sequence of SEQ ID NO: 524. In some embodiments, the SAM, LNP, PNP, or nanoemulsion comprises a polynucleotide that is at least 100% identical to the nucleic acid sequence of SEQ ID NO: 524. In some embodiments, the SAM, LNP, PNP, or nanoemulsion comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 524. In some embodiments, the SAM, LNP, PNP, or nanoemulsion comprises a polynucleotide that comprises 15 or fewer nucleotide mismatches to the nucleic acid sequence of SEQ ID NO: 524. In some embodiments, the SAM, LNP, PNP, or nanoemulsion comprises a polynucleotide that comprises 10 or fewer nucleotide mismatches to the nucleic acid sequence of SEQ ID NO: 524. In some embodiments, the SAM, LNP, PNP, or nanoemulsion comprises a polynucleotide that comprises 5 or fewer nucleotide mismatches to the nucleic acid sequence of SEQ ID NO: 524. In some embodiments, the SAM, LNP, PNP, or nanoemulsion comprises a polynucleotide that comprises 4 or fewer nucleotide mismatches to the nucleic acid sequence of SEQ ID NO: 524. In some embodiments, the SAM, LNP, PNP, or nanoemulsion comprises a polynucleotide that comprises 3 or fewer nucleotide mismatches to the nucleic acid sequence of SEQ ID NO: 524. In some embodiments, the SAM, LNP, PNP, or nanoemulsion comprises a polynucleotide that comprises 2 or fewer nucleotide mismatches to the nucleic acid sequence of SEQ ID NO: 524. In some embodiments, the SAM, LNP, PNP, or nanoemulsion comprises a polynucleotide that comprises 1 or fewer nucleotide mismatches to the nucleic acid sequence of SEQ ID NO: 524. In some embodiments, the SAM, LNP, PNP, or nanoemulsion comprises a polynucleotide that comprises a codon-optimized nucleic acid sequence of SEQ ID NO: 524.

In various embodiments, the SAM, LNP, PNP, or nanoemulsion comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 525. In some embodiments, the SAM, LNP, PNP, or nanoemulsion comprises a polynucleotide that is at least 85% identical to the nucleic acid sequence of SEQ ID NO: 525. In some embodiments, the SAM, LNP, PNP, or nanoemulsion comprises a polynucleotide that is at least 90% identical to the nucleic acid sequence of SEQ ID NO: 525. In some embodiments, the SAM, LNP, PNP, or nanoemulsion comprises a polynucleotide that is at least 95% identical to the nucleic acid sequence of SEQ ID NO: 525. In some embodiments, the SAM, LNP, PNP, or nanoemulsion comprises a polynucleotide that is at least 97% identical to the nucleic acid sequence of SEQ ID NO: 525. In some embodiments, the SAM, LNP, PNP, or nanoemulsion comprises a polynucleotide that is at least 98% identical to the nucleic acid sequence of SEQ ID NO: 525. In some embodiments, the SAM, LNP, PNP, or nanoemulsion comprises a polynucleotide that is at least 99% identical to the nucleic acid sequence of SEQ ID NO: 525. In some embodiments, the SAM, LNP, PNP, or nanoemulsion comprises a polynucleotide that is at least 100% identical to the nucleic acid sequence of SEQ ID NO: 525. In some embodiments, the SAM, LNP, PNP, or nanoemulsion comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 525. In some embodiments, the SAM, LNP, PNP, or nanoemulsion comprises a polynucleotide that comprises 15 or fewer nucleotide mismatches to the nucleic acid sequence of SEQ ID NO: 525. In some embodiments, the SAM, LNP, PNP, or nanoemulsion comprises a polynucleotide that comprises 10 or fewer nucleotide mismatches to the nucleic acid sequence of SEQ ID NO: 525. In some embodiments, the SAM, LNP, PNP, or nanoemulsion comprises a polynucleotide that comprises 5 or fewer nucleotide mismatches to the nucleic acid sequence of SEQ ID NO: 525. In some embodiments, the SAM, LNP, PNP, or nanoemulsion comprises a polynucleotide that comprises 4 or fewer nucleotide mismatches to the nucleic acid sequence of SEQ ID NO: 525. In some embodiments, the SAM, LNP, PNP, or nanoemulsion comprises a polynucleotide that comprises 3 or fewer nucleotide mismatches to the nucleic acid sequence of SEQ ID NO: 525. In some embodiments, the SAM, LNP, PNP, or nanoemulsion comprises a polynucleotide that comprises 2 or fewer nucleotide mismatches to the nucleic acid sequence of SEQ ID NO: 525. In some embodiments, the SAM, LNP, PNP, or nanoemulsion comprises a polynucleotide that comprises 1 or fewer nucleotide mismatches to the nucleic acid sequence of SEQ ID NO: 525. In some embodiments, the SAM, LNP, PNP, or nanoemulsion comprises a polynucleotide that comprises a codon-optimized nucleic acid sequence of SEQ ID NO: 525.

In various embodiments, the SAM, LNP, PNP, or nanoemulsion comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 526. In some embodiments, the SAM, LNP, PNP, or nanoemulsion comprises a polynucleotide that is at least 85% identical to the nucleic acid sequence of SEQ ID NO: 526. In some embodiments, the SAM, LNP, PNP, or nanoemulsion comprises a polynucleotide that is at least 90% identical to the nucleic acid sequence of SEQ ID NO: 526. In some embodiments, the SAM, LNP, PNP, or nanoemulsion comprises a polynucleotide that is at least 95% identical to the nucleic acid sequence of SEQ ID NO: 526. In some embodiments, the SAM, LNP, PNP, or nanoemulsion comprises a polynucleotide that is at least 97% identical to the nucleic acid sequence of SEQ ID NO: 526. In some embodiments, the SAM, LNP, PNP, or nanoemulsion comprises a polynucleotide that is at least 98% identical to the nucleic acid sequence of SEQ ID NO: 526. In some embodiments, the SAM, LNP, PNP, or nanoemulsion comprises a polynucleotide that is at least 99% identical to the nucleic acid sequence of SEQ ID NO: 526. In some embodiments, the SAM, LNP, PNP, or nanoemulsion comprises a polynucleotide that is at least 100% identical to the nucleic acid sequence of SEQ ID NO: 526. In some embodiments, the SAM, LNP, PNP, or nanoemulsion comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 526. In some embodiments, the SAM, LNP, PNP, or nanoemulsion comprises a polynucleotide that comprises 15 or fewer nucleotide mismatches to the nucleic acid sequence of SEQ ID NO: 526. In some embodiments, the SAM, LNP, PNP, or nanoemulsion comprises a polynucleotide that comprises 10 or fewer nucleotide mismatches to the nucleic acid sequence of SEQ ID NO: 526. In some embodiments, the SAM, LNP, PNP, or nanoemulsion comprises a polynucleotide that comprises 5 or fewer nucleotide mismatches to the nucleic acid sequence of SEQ ID NO: 526. In some embodiments, the SAM, LNP, PNP, or nanoemulsion comprises a polynucleotide that comprises 4 or fewer nucleotide mismatches to the nucleic acid sequence of SEQ ID NO: 526. In some embodiments, the SAM, LNP, PNP, or nanoemulsion comprises a polynucleotide that comprises 3 or fewer nucleotide mismatches to the nucleic acid sequence of SEQ ID NO: 526. In some embodiments, the SAM, LNP, PNP, or nanoemulsion comprises a polynucleotide that comprises 2 or fewer nucleotide mismatches to the nucleic acid sequence of SEQ ID NO: 526. In some embodiments, the SAM, LNP, PNP, or nanoemulsion comprises a polynucleotide that comprises 1 or fewer nucleotide mismatches to the nucleic acid sequence of SEQ ID NO: 526. In some embodiments, the SAM, LNP, PNP, or nanoemulsion comprises a polynucleotide that comprises a codon-optimized nucleic acid sequence of SEQ ID NO: 526.

In various embodiments, the SAM, LNP, PNP, or nanoemulsion comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 522. In some embodiments, the SAM, LNP, PNP, or nanoemulsion comprises a polynucleotide that is at least 85% identical to the nucleic acid sequence of SEQ ID NO: 522. In some embodiments, the SAM, LNP, PNP, or nanoemulsion comprises a polynucleotide that is at least 90% identical to the nucleic acid sequence of SEQ ID NO: 522. In some embodiments, the SAM, LNP, PNP, or nanoemulsion comprises a polynucleotide that is at least 95% identical to the nucleic acid sequence of SEQ ID NO: 522. In some embodiments, the SAM, LNP, PNP, or nanoemulsion comprises a polynucleotide that is at least 97% identical to the nucleic acid sequence of SEQ ID NO: 522. In some embodiments, the SAM, LNP, PNP, or nanoemulsion comprises a polynucleotide that is at least 98% identical to the nucleic acid sequence of SEQ ID NO: 522. In some embodiments, the SAM, LNP, PNP, or nanoemulsion comprises a polynucleotide that is at least 99% identical to the nucleic acid sequence of SEQ ID NO: 522. In some embodiments, the SAM, LNP, PNP, or nanoemulsion comprises a polynucleotide that is at least 100% identical to the nucleic acid sequence of SEQ ID NO: 522. In some embodiments, the SAM, LNP, PNP, or nanoemulsion comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 522. In some embodiments, the SAM, LNP, PNP, or nanoemulsion comprises a polynucleotide that comprises 15 or fewer nucleotide mismatches to the nucleic acid sequence of SEQ ID NO: 522. In some embodiments, the SAM, LNP, PNP, or nanoemulsion comprises a polynucleotide that comprises 10 or fewer nucleotide mismatches to the nucleic acid sequence of SEQ ID NO: 522. In some embodiments, the SAM, LNP, PNP, or nanoemulsion comprises a polynucleotide that comprises 5 or fewer nucleotide mismatches to the nucleic acid sequence of SEQ ID NO: 522. In some embodiments, the SAM, LNP, PNP, or nanoemulsion comprises a polynucleotide that comprises 4 or fewer nucleotide mismatches to the nucleic acid sequence of SEQ ID NO: 522. In some embodiments, the SAM, LNP, PNP, or nanoemulsion comprises a polynucleotide that comprises 3 or fewer nucleotide mismatches to the nucleic acid sequence of SEQ ID NO: 522. In some embodiments, the SAM, LNP, PNP, or nanoemulsion comprises a polynucleotide that comprises 2 or fewer nucleotide mismatches to the nucleic acid sequence of SEQ ID NO: 522. In some embodiments, the SAM, LNP, PNP, or nanoemulsion comprises a polynucleotide that comprises 1 or fewer nucleotide mismatches to the nucleic acid sequence of SEQ ID NO: 522. In some embodiments, the SAM, LNP, PNP, or nanoemulsion comprises a polynucleotide that comprises a codon-optimized nucleic acid sequence of SEQ ID NO: 522.

In various embodiments, the SAM, LNP, PNP, or nanoemulsion comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 523. In some embodiments, the SAM, LNP, PNP, or nanoemulsion comprises a polynucleotide that is at least 85% identical to the nucleic acid sequence of SEQ ID NO: 523. In some embodiments, the SAM, LNP, PNP, or nanoemulsion comprises a polynucleotide that is at least 90% identical to the nucleic acid sequence of SEQ ID NO: 523. In some embodiments, the SAM, LNP, PNP, or nanoemulsion comprises a polynucleotide that is at least 95% identical to the nucleic acid sequence of SEQ ID NO: 523. In some embodiments, the SAM, LNP, PNP, or nanoemulsion comprises a polynucleotide that is at least 97% identical to the nucleic acid sequence of SEQ ID NO: 523. In some embodiments, the SAM, LNP, PNP, or nanoemulsion comprises a polynucleotide that is at least 98% identical to the nucleic acid sequence of SEQ ID NO: 523. In some embodiments, the SAM, LNP, PNP, or nanoemulsion comprises a polynucleotide that is at least 99% identical to the nucleic acid sequence of SEQ ID NO: 523. In some embodiments, the SAM, LNP, PNP, or nanoemulsion comprises a polynucleotide that is at least 100% identical to the nucleic acid sequence of SEQ ID NO: 523. In some embodiments, the SAM, LNP, PNP, or nanoemulsion comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 523. In some embodiments, the SAM, LNP, PNP, or nanoemulsion comprises a polynucleotide that comprises 15 or fewer nucleotide mismatches to the nucleic acid sequence of SEQ ID NO: 523. In some embodiments, the SAM, LNP, PNP, or nanoemulsion comprises a polynucleotide that comprises 10 or fewer nucleotide mismatches to the nucleic acid sequence of SEQ ID NO: 523. In some embodiments, the SAM, LNP, PNP, or nanoemulsion comprises a polynucleotide that comprises 5 or fewer nucleotide mismatches to the nucleic acid sequence of SEQ ID NO: 523. In some embodiments, the SAM, LNP, PNP, or nanoemulsion comprises a polynucleotide that comprises 4 or fewer nucleotide mismatches to the nucleic acid sequence of SEQ ID NO: 523. In some embodiments, the SAM, LNP, PNP, or nanoemulsion comprises a polynucleotide that comprises 3 or fewer nucleotide mismatches to the nucleic acid sequence of SEQ ID NO: 523. In some embodiments, the SAM, LNP, PNP, or nanoemulsion comprises a polynucleotide that comprises 2 or fewer nucleotide mismatches to the nucleic acid sequence of SEQ ID NO: 523. In some embodiments, the SAM, LNP, PNP, or nanoemulsion comprises a polynucleotide that comprises 1 or fewer nucleotide mismatches to the nucleic acid sequence of SEQ ID NO: 523. In some embodiments, the SAM, LNP, PNP, or nanoemulsion comprises a polynucleotide that comprises a codon-optimized nucleic acid sequence of SEQ ID NO: 523.

In various embodiments, the LNP, PNP, or nanoemulsion comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 520. In some embodiments, the LNP, PNP, or nanoemulsion comprises a polynucleotide that is at least 85% identical to the nucleic acid sequence of SEQ ID NO: 520. In some embodiments, the LNP, PNP, or nanoemulsion comprises a polynucleotide that is at least 90% identical to the nucleic acid sequence of SEQ ID NO: 520. In some embodiments, the LNP, PNP, or nanoemulsion comprises a polynucleotide that is at least 95% identical to the nucleic acid sequence of SEQ ID NO: 520. In some embodiments, the LNP, PNP, or nanoemulsion comprises a polynucleotide that is at least 97% identical to the nucleic acid sequence of SEQ ID NO: 520. In some embodiments, the LNP, PNP, or nanoemulsion comprises a polynucleotide that is at least 98% identical to the nucleic acid sequence of SEQ ID NO: 520. In some embodiments, the LNP, PNP, or nanoemulsion comprises a polynucleotide that is at least 99% identical to the nucleic acid sequence of SEQ ID NO: 520. In some embodiments, the LNP, PNP, or nanoemulsion comprises a polynucleotide that is at least 100% identical to the nucleic acid sequence of SEQ ID NO: 520. In some embodiments, the LNP, PNP, or nanoemulsion comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 520. In some embodiments, the LNP, PNP, or nanoemulsion comprises a polynucleotide that comprises 15 or fewer nucleotide mismatches to the nucleic acid sequence of SEQ ID NO: 520. In some embodiments, the LNP, PNP, or nanoemulsion comprises a polynucleotide that comprises 10 or fewer nucleotide mismatches to the nucleic acid sequence of SEQ ID NO: 520. In some embodiments, the LNP, PNP, or nanoemulsion comprises a polynucleotide that comprises 5 or fewer nucleotide mismatches to the nucleic acid sequence of SEQ ID NO: 520. In some embodiments, the LNP, PNP, or nanoemulsion comprises a polynucleotide that comprises 4 or fewer nucleotide mismatches to the nucleic acid sequence of SEQ ID NO: 520. In some embodiments, the LNP, PNP, or nanoemulsion comprises a polynucleotide that comprises 3 or fewer nucleotide mismatches to the nucleic acid sequence of SEQ ID NO: 520. In some embodiments, the LNP, PNP, or nanoemulsion comprises a polynucleotide that comprises 2 or fewer nucleotide mismatches to the nucleic acid sequence of SEQ ID NO: 520. In some embodiments, the LNP, PNP, or nanoemulsion comprises a polynucleotide that comprises 1 or fewer nucleotide mismatches to the nucleic acid sequence of SEQ ID NO: 520. In some embodiments, the LNP, PNP, or nanoemulsion comprises a polynucleotide that comprises a codon-optimized nucleic acid sequence of SEQ ID NO: 520.

In various embodiments, the LNP, PNP, or nanoemulsion comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 521. In some embodiments, the LNP, PNP, or nanoemulsion comprises a polynucleotide that is at least 85% identical to the nucleic acid sequence of SEQ ID NO: 521. In some embodiments, the LNP, PNP, or nanoemulsion comprises a polynucleotide that is at least 90% identical to the nucleic acid sequence of SEQ ID NO: 521. In some embodiments, the LNP, PNP, or nanoemulsion comprises a polynucleotide that is at least 95% identical to the nucleic acid sequence of SEQ ID NO: 521. In some embodiments, the LNP, PNP, or nanoemulsion comprises a polynucleotide that is at least 97% identical to the nucleic acid sequence of SEQ ID NO: 521. In some embodiments, the LNP, PNP, or nanoemulsion comprises a polynucleotide that is at least 98% identical to the nucleic acid sequence of SEQ ID NO: 521. In some embodiments, the LNP, PNP, or nanoemulsion comprises a polynucleotide that is at least 99% identical to the nucleic acid sequence of SEQ ID NO: 521. In some embodiments, the LNP, PNP, or nanoemulsion comprises a polynucleotide that is at least 100% identical to the nucleic acid sequence of SEQ ID NO: 521. In some embodiments, the LNP, PNP, or nanoemulsion comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 521. In some embodiments, the LNP, PNP, or nanoemulsion comprises a polynucleotide that comprises 15 or fewer nucleotide mismatches to the nucleic acid sequence of SEQ ID NO: 521. In some embodiments, the LNP, PNP, or nanoemulsion comprises a polynucleotide that comprises 10 or fewer nucleotide mismatches to the nucleic acid sequence of SEQ ID NO: 521. In some embodiments, the LNP, PNP, or nanoemulsion comprises a polynucleotide that comprises 5 or fewer nucleotide mismatches to the nucleic acid sequence of SEQ ID NO: 521. In some embodiments, the LNP, PNP, or nanoemulsion comprises a polynucleotide that comprises 4 or fewer nucleotide mismatches to the nucleic acid sequence of SEQ ID NO: 521. In some embodiments, the LNP, PNP, or nanoemulsion comprises a polynucleotide that comprises 3 or fewer nucleotide mismatches to the nucleic acid sequence of SEQ ID NO: 521. In some embodiments, the LNP, PNP, or nanoemulsion comprises a polynucleotide that comprises 2 or fewer nucleotide mismatches to the nucleic acid sequence of SEQ ID NO: 521. In some embodiments, the LNP, PNP, or nanoemulsion comprises a polynucleotide that comprises 1 or fewer nucleotide mismatches to the nucleic acid sequence of SEQ ID NO: 521. In some embodiments, the LNP, PNP, or nanoemulsion comprises a polynucleotide that comprises a codon-optimized nucleic acid sequence of SEQ ID NO: 521.

### Compositions

Provided are compositions, such as pharmaceutical compositions or immunogenic compositions, comprising one or more of the fusion polypeptides, as described herein, or a polynucleotide encoding one or more of the fusion polypeptides, as described herein, or an expression vector, viral expression vector, or viral vector comprising one or more of such polynucleotides, or a LNP, PNP, or nanoemulsion comprising one or more of such polynucleotides. The composition may further comprise a pharmaceutically acceptable diluent, carrier or excipient. Generally, the compositions described herein are immunogenic. In certain embodiments, the composition comprises a therapeutically effective amount of the one or more of the fusion polypeptides, as described herein, or a polynucleotide encoding one or more of the fusion polypeptides, as described herein, or an expression vector, viral expression vector, or viral vector comprising one or more of such polynucleotides, or a LNP, PNP, or nanoemulsion comprising one or more of such polynucleotides.

Various pharmaceutically acceptable diluents, carriers, and excipients, and techniques for the preparation and use of pharmaceutical compositions will be known to those of skill in the art in light of the present disclosure. Illustrative pharmaceutical compositions and pharmaceutically acceptable diluents, carriers, and excipients are also described in, e.g., Loyd V. Allen Jr (Editor), “Remington: The Science and Practice of Pharmacy,” 22^{nd} Edition, 2012, Pharmaceutical Press; Brunton, Knollman and Hilal-Dandan, “Goodman and Gilman's The Pharmacological Basis of Therapeutics,” 13th Edition, 2017, McGraw-Hill Education/Medical; McNally and Hastedt (Editors), “Protein Formulation and Delivery, 2nd Edition, 2007, CRC Press; Banga, “Therapeutic Peptides and Proteins: Formulation, Processing, and Delivery Systems,” 3rd Edition, 2015, CRC Press; Lars Hovgaard, Frokjaer and van de Weert (Editors), “Pharmaceutical Formulation Development of Peptides and Proteins,” 2nd Edition, 2012, CRC Press; Carpenter and Manning (Editors), “Rational Design of Stable Protein Formulations: Theory and Practice,” 2002, Springer (Pharmaceutical Biotechnology (Book 13)); Meyer (Editor), “Therapeutic Protein Drug Products: Practical Approaches to Formulation in the Laboratory, Manufacturing, and the Clinic, 2012, Woodhead Publishing.

In embodiments where the fusion polypeptides are expressed from a viral expression vector, the viral expression vector can be formulated for the desired route of administration, e.g., as an isotonic pharmaceutically acceptable aqueous solution for intravenous, intramuscular, subcutaneous or intradermal administration. In some embodiments, the viral expression vector can be formulated for mucosal, e.g., buccal, intranasal or intrarectal delivery. Illustrative formulations for viral expression vectors that can be used in the herein described pharmaceutical compositions and methods are described, e.g., in Manfredsson and Benskey, editors, “Viral Vectors for Gene Therapy: Methods and Protocols (Methods in Molecular Biology),” 2019, Book 1937 in Methods in Molecular Biology Series, Humana Press; WO 2017/013169 (formulation of Adenoviral vectors in an aqueous mixture or freeze dried composition in the presence of amorphous sugar and low salt concentration); and Kumru, et al., J Pharm Sci. (2018) November; 107 (11): 2764-2774 (aqueous formulations buffered in Tris and containing proline, lactose, and mannitol as stabilizing additives). Formulation of arenavirus vectors is described, e.g., in WO 2009/083210; WO 2016/075250 and WO 2017/198726. In certain embodiments, the viral expression vectors are delivered via microneedle-mediated delivery, e.g., as described in Zaric, et al., Expert Opin Drug Deliv. (2017) October; 14 (10): 1177-1187. Intranasal viral vaccination by administration of viral particles to the nares is described, e.g., in Dorta-Estremera, et al., PLoS One. 2017 Dec. 8; 12 (12): e0188807. Intrarectal viral vaccination by administration of viral particles to the rectum is described, e.g., in Patterson, et al., Clin Vaccine Immunol. (2012) May; 19 (5): 629-37.

In some embodiments, each carrier, diluent or excipient is “acceptable” in the sense of being compatible with the other ingredients of the pharmaceutical composition and not injurious to the subject. Often, the pharmaceutically acceptable carrier is an aqueous pH-buffered solution. Some examples of materials which can serve as pharmaceutically-acceptable carriers, diluents or excipients include: water; buffers, e.g., a buffer having a pKa in the range of about 6.0 to about 8.0, e.g., a physiologically acceptable buffer, e.g., selected from phosphate, carbonate, bicarbonate, citrate, maleate, glycine-glycine, HEPES, HEPPSO, HEPPS, imidazole, BICINE, TRICINE, Tris, and BIS-Tris; sugars, such as lactose, trehalose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Hank's solution, Ringer's solution; ethyl alcohol; phosphate buffer solutions; amino acids (e.g., charged amino acids, including without limitation, aspartate, asparagine, glutamate, glutamine, histidine, arginine, lysine); and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Solid and semi-solid formulations that can be used for intravaginal or intrarectal (e.g., in the form of a troche, a pessary or a suppository) delivery of viral expression vectors, virosomes or virus-like particles (VLPs) is described, e.g., in Brown, et al., PLoS One. 2017 Aug. 17; 12 (8): e0183510; Brown, et al., PLoS One. 2016 Mar. 10; 11 (3): e0151184; and Amacker, et al., npj Vaccines 5, 41 (2020).

In one particular formulation, an adenovirus vector (e.g., a ChAd vector) described herein is formulated in an isotonic aqueous solution comprising a biologically compatible buffer having a pKa in the range of about 6.0 to about 8.0 (e.g., HEPES and NaCl), at a neutral or near-neutral pH and a non-ionic surfactant (e.g., PLURONIC® F68 (a.k.a., poloxamer 188)). In one particular formulation, an arenavirus vector (e.g., a LCMV or Pichinde mammarenavirus vector) described herein is formulated in an isotonic aqueous solution comprising HEPES buffer at pH 7.4, NaCl, and PLURONIC® F68 (a.k.a., poloxamer 188). Schleiss, et al. (Clin Vaccine Immunol. 2017 Jan. 5; 24 (1): e00300-16) describes an LCMV formulating LCMV vectors in a diluent of 25 mM HEPES, 150 mM NaCl, 0.01% PLURONIC® F68; pH 7.4), which can be used to formulate the herein described arenavirus vectors. A final concentration of 10% sorbitol was added before freezing below −60° C.

In another particular formulation, an adenovirus vector (e.g., a ChAd vector) described herein is formulated in an aqueous buffer containing 5 mM Tris, 5% sucrose, 75 mM sodium chloride, and 1 mM magnesium chloride, at pH 8.0. In some embodiments, the nominal concentration of the adenovirus vector is 5×10¹¹ vp/mL. In some embodiments, the adenovirus vector comprises the nucleotide sequence of SEQ ID NO: 520. In some embodiments, the adenovirus vector comprises the nucleotide sequence of SEQ ID NO: 521. In some embodiments, the adenovirus vector comprises an immunogenic polypeptide encoded by a nucleotide sequence of any one of SEQ ID NOs: 524 to 526. In some embodiments, the adenovirus vector comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the HIV immunogen nucleic acid sequences of SEQ ID NOs: 524 to 526, wherein percent identity is across the full length of the HIV immunogen nucleic acid sequence of SEQ ID NOs: 524 to 526. In some embodiments, the adenovirus vector comprises an immunogenic nucleotide sequence that encodes an immunogenic polypeptide of any one of SEQ ID NOs: 527 to 528. In some embodiments, the adenovirus vector comprises a polynucleotide encoding a polypeptide sequence that is at least 80%, 85%, 86%, 87%, 88%, 89%, 90%, 91%, 92%, 93%, 94%, 95%, 96%, 97%, 98%, 99%, or 100% identical to any one of the polypeptide sequences of SEQ ID NOS: 527 to 528, wherein percent identity is across the full length of the HIV immunogen nucleic acid sequence of SEQ ID NOs: 527 to 528.

In another particular formulation, a SAM described herein is formulated in a suspension of LNPs in an aqueous buffer containing 5 mM Tris, 10% sucrose, and 10% maltose, at pH 8.0. The product is formulated to deliver 0.2 mg of SAM per mL of suspension. In some embodiments, the SAM comprises the nucleotide sequence of SEQ ID NO: 522. In some embodiments, the SAM comprises the nucleotide sequence of SEQ ID NO: 523. In some embodiments, the SAM comprises an immunogenic polypeptide encoded by a nucleotide sequence of any one of SEQ ID NOs: 524 to 526. In some embodiments, the SAM comprises a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the HIV immunogen nucleic acid sequences of SEQ ID NOs: 524 to 526, wherein percent identity is across the full length of the HIV immunogen nucleic acid sequence of SEQ ID NOs: 524 to 526. In some embodiments, the SAM comprises an immunogenic nucleotide sequence that encodes an immunogenic polypeptide of any one of SEQ ID NOs: 527 to 528. In some embodiments, the SAM comprises a polynucleotide encoding a polypeptide sequence that is at least 80%, 85%, 86%, 87%, 88%, 89%, 90%, 91%, 92%, 93%, 94%, 95%, 96%, 97%, 98%, 99%, or 100% identical to any one of the polypeptide sequences of SEQ ID NOs: 527 to 528, wherein percent identity is across the full length of the HIV immunogen nucleic acid sequence of SEQ ID NOs: 527 to 528.

The formulation of and delivery methods of pharmaceutical compositions will generally be adapted according to the site and the disease to be treated. Exemplary formulations include without limitation, those suitable for parenteral administration, e.g., intravenous, intra-arterial, intramuscular, or subcutaneous administration, including formulations encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays. In some embodiments, the pharmaceutical compositions are formulated for parenteral, e.g., intravenous, subcutaneous, or oral administration. In some embodiments, the pharmaceutical compositions are formulated for mucosal, e.g., buccal, intranasal, intrarectal and/or intravaginal administration.

In certain embodiments, pharmaceutical compositions are sterile. In certain embodiments, the pharmaceutical composition has a pH in the range of 4.5 to 8.5, 4.5 to 6.5, 6.5 to 8.5, or a pH of about 5.0, about 5.5, about 6.0, about 6.5, about 7.0, about 7.5, about 8.0 or about 8.5. In one embodiment, the pharmaceutical composition has an osmolarity in the range of 240-260 or 250-330 mOsmol/L. In certain embodiments, the pharmaceutical composition is isotonic or near isotonic.

In some embodiments, the pharmaceutical compositions are liquids or solids. In some embodiments, the pharmaceutical composition comprises an aqueous solution. In some embodiments, the pharmaceutical composition is lyophilized or is a frozen liquid.

In some embodiments, the pharmaceutical composition further comprises one or more additional therapeutic agents, e.g., a second therapeutic agent, or second and third therapeutic agents, for use in combination therapies, as described herein.

In certain embodiments, the pharmaceutical composition further comprises an adjuvant. Illustrative adjuvants that can be co-formulated or co-administered with the herein described fusion polypeptides, polynucleotides encoding such fusion polypeptides and vectors expressing such fusion polypeptides include without limitation cytokines, chemokines, immune costimulatory molecules, toll-like receptor agonists or inhibitors of immune suppressive pathways, as described herein, and in Li, et al., Curr Issues Mol Biol. (2017) 22:17-40. Other adjuvants that can be co-formulated or co-administered with the herein described fusion polypeptides, polynucleotides encoding such fusion polypeptides and vectors expressing such fusion polypeptides include without limitation mineral salts (e.g., aluminum salts (e.g., alum), calcium phosphate, incomplete Freunds's adjuvant), lipid particles (e.g., MF59, cochleates, virus-like particles), microparticles (e.g., virosomes, polylactic acid (PLA), poly[lactide-coglycolide](PLG)), immune potentiators (e.g., dsRNA:Poly(I:C), Poly-IC:LC, Monophosphoryl lipid A (MPL), LPS, Flagellin, Imidazoquinolines: imiquimod (R837), resiquimod (848), CpG oligodeoxynucleotides (ODN), Muramyl dipeptide (MDP), Saponins (QS-21)), and mucosal adjuvants (e.g., Cholera toxin (CT), Heat-labile enterotoxin (LTK3 and LTR72), Chitosan). Adjuvants that can be co-formulated or co-administered with the herein described fusion polypeptides, polynucleotides encoding such fusion polypeptides and vectors expressing such fusion polypeptides are summarized in Apostólico, et al., J Immunol Res. (2016) 2016:1459394.

In certain embodiments, the pharmaceutical composition further comprises an immunomodulator. Illustrative immunomodulators that can be co-formulated or co-administered with the herein described fusion polypeptides, polynucleotides encoding such fusion polypeptides and vectors expressing such fusion polypeptides include without limitation toll-like receptor agonists and small molecule immune checkpoint inhibitors. Example TLR7 agonists that can be co-formulated or co-administered include without limitation AL-034, DSP-0509, GS-9620 (vesatolimod), LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854 and RG-7795. Illustrative TLR7/TLR8 agonists that can be co-formulated or co-administered include CV8102, NKTR-262, telratolimod and BDB-001. Example TLR8 agonists that can be co-formulated or co-administered include without limitation E-6887, IMO-4200, IMO-8400, IMO-9200, MCT-465, MEDI-9197, motolimod, resiquimod, GS-9688, VTX-1463, VTX-763, 3M-051, 3M-052. Example TLR9 agonists that can be co-formulated or co-administered include without limitation AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of small molecule inhibitors of CD274 or PDCD1 that can be co-formulated or co-administered include without limitation GS-4224, GS-4416, INCB086550 and MAX10181. An example small molecule inhibitor of CTLA4 that can be co-formulated or co-administered includes BPI-002.

In some embodiments, the pharmaceutical compositions or immunogenic compositions comprise mixtures of two or more fusion polypeptides, two or more polynucleotides encoding such fusion polypeptides, or two or more vectors expressing such fusion polypeptides. For example, in certain embodiments, the mixtures comprise bivalent pairs of fusion polypeptides, as described herein. In some embodiments, the pharmaceutical composition comprises two or more fusion polypeptides, two or more polynucleotides encoding such fusion polypeptides, or two or more vectors expressing such fusion polypeptides, the fusion polypeptides comprising or consisting of the following polypeptide segments in sequential order, from N-terminus to C-terminus, optionally joined or connected by one or more linkers: SEQ ID NOs: 70, 76, 94, 151 and 161; and SEQ ID NOs: 71, 77, 95, 152 and 162. In some embodiments, the pharmaceutical composition comprises two or more fusion polypeptides, two or more polynucleotides encoding such fusion polypeptides, or two or more vectors expressing such fusion polypeptides, the fusion polypeptides comprising or consisting of the following polypeptide segments in sequential order, from N-terminus to C-terminus, optionally joined or connected by one or more linkers: SEQ ID NOs: 188, 305, 28, 41, 294, 4, 176, 11, 319, 259, 282, 223, 213 and 37; SEQ ID NOs: 188, 305, 28, 41 and 294; SEQ ID NOs: 4, 176, 11, 319, 259, 282, 223, 213 and 37; SEQ ID NOs: 189, 306, 29, 42, 295, 5, 177, 12, 320, 260, 283, 224, 214 and 38; SEQ ID NOs: 189, 306, 29, 42 and 295; SEQ ID NOs: 5, 177, 12, 320, 260, 283, 224, 214 and 38; SEQ ID NOs: 305, 319, 259, 282, 223, 213, 294, 176 and 188; SEQ ID NOs: 306, 320, 260, 283, 224, 214, 295, 177 and 189; SEQ ID NOs: 305, 294, 223, 213, 176, 259, 319, 188 and 282; SEQ ID NOs: 306, 295, 224, 214, 177, 260, 320, 189 and 283; SEQ ID NOs: 305, 294, 319, 259, 282, 223, 176, and 188; SEQ ID NOs: 306, 295, 320, 260, 283, 224, 177 and 189; SEQ ID NOs: 305, 223, 294, 176, 259, 319, 188 and 282; and SEQ ID NOs: 306, 224, 295, 177, 260, 320, 189 and 283. In some embodiments, the pharmaceutical composition comprises two or more fusion polypeptides, two or more polynucleotides encoding such fusion polypeptides, or two or more vectors expressing such fusion polypeptides, the fusion polypeptides comprising or consisting of the following polypeptide segments in sequential order, from N-terminus to C-terminus, optionally joined or connected by one or more linkers: SEQ ID NOs: 76, 86, 94, 180, 186, 221, 294, 307, 321 and 151; and SEQ ID NOs: 77, 87, 95, 181, 187, 222, 295, 308, 322 and 152.

In some embodiments, the pharmaceutical composition or immunogenic composition comprises two or more fusion polypeptides, two or more polynucleotides encoding such fusion polypeptides, or two or more vectors expressing such fusion polypeptides, the fusion polypeptides comprising or consisting of an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430. In some embodiments, the pharmaceutical composition or immunogenic composition comprises two or more fusion polypeptides, two or more polynucleotides encoding such fusion polypeptides, or two or more vectors expressing such fusion polypeptides, the fusion polypeptides comprising or consisting of an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410. In some embodiments, the pharmaceutical composition or immunogenic composition comprises two or more fusion polypeptides, two or more polynucleotides encoding such fusion polypeptides, or two or more vectors expressing such fusion polypeptides, the fusion polypeptides comprising or consisting of an amino acid sequence of any one of SEQ ID NOS: 527-528, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 527-528.

In some embodiments, the pharmaceutical composition or immunogenic composition comprises two or more fusion polypeptides, two or more polynucleotides encoding such fusion polypeptides, or two or more vectors expressing such fusion polypeptides, the fusion polypeptides comprising or consisting of an amino acid sequence of any one of SEQ ID NOs: 345-350 and SEQ ID NOs: 422-424, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NO: 345-350 and SEQ ID NOs: 422-424.

In some embodiments, the pharmaceutical compositions or immunogenic compositions comprise a first fusion polypeptide or polynucleotide encoding such fusion polypeptide or a vector expressing such fusion polypeptide, the fusion polypeptide comprising one or more polypeptide segments encoded by HIV-1 Gag and Nef genes and a second fusion polypeptide or polynucleotide encoding such fusion polypeptide or viral expression vector expressing such fusion polypeptide, the fusion polypeptide comprising one or more polypeptide segments encoded by HIV-1 Pol or Pol and Env genes. In some embodiments, the pharmaceutical composition or immunogenic composition comprises (1) one or more fusion polypeptides or one or more polynucleotides encoding such fusion polypeptides or one or more vectors expressing such fusion polypeptides, the fusion polypeptide comprising or consisting of the following polypeptide segments in sequential order, from N-terminus to C-terminus, optionally joined or connected by one or more linkers: SEQ ID NOs: 70, 76, 94, 151 and 161; or SEQ ID NOs: 71, 77, 95, 152 and 162; and (2) one or more fusion polypeptides or one or more polynucleotides encoding such fusion polypeptides or one or more vectors expressing such fusion polypeptides, the fusion polypeptide comprising or consisting of the following polypeptide segments in sequential order, from N-terminus to C-terminus, optionally joined or connected by one or more linkers: SEQ ID NOs: 188, 305, 28, 41, 294, 4, 176, 11, 319, 259, 282, 223, 213 and 37; SEQ ID NOs: 188, 305, 28, 41 and 294; SEQ ID NOs: 4, 176, 11, 319, 259, 282, 223, 213 and 37; SEQ ID NOs: 189, 306, 29, 42, 295, 5, 177, 12, 320, 260, 283, 224, 214 and 38; SEQ ID NOs: 189, 306, 29, 42 and 295; SEQ ID NOs: 5, 177, 12, 320, 260, 283, 224, 214 and 38; SEQ ID NOs: 305, 319, 259, 282, 223, 213, 294, 176 and 188; SEQ ID NOs: 306, 320, 260, 283, 224, 214, 295, 177 and 189; SEQ ID NOs: 305, 294, 223, 213, 176, 259, 319, 188 and 282; SEQ ID NOs: 306, 295, 224, 214, 177, 260, 320, 189 and 283; SEQ ID NOs: 305, 294, 319, 259, 282, 223, 176, and 188; SEQ ID NOs: 306, 295, 320, 260, 283, 224, 177 and 189; SEQ ID NOs: 305, 223, 294, 176, 259, 319, 188 and 282; or SEQ ID NOs: 306, 224, 295, 177, 260, 320, 189 and 283. In some embodiments, the pharmaceutical composition or immunogenic composition comprises (1) one or more fusion polypeptides or one or more polynucleotides encoding such fusion polypeptides or one or more vectors expressing such fusion polypeptides, the fusion polypeptide comprising an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430; and (2) one or more fusion polypeptides or one or more polynucleotides encoding such fusion polypeptides or one or more vectors expressing such fusion polypeptides, the fusion polypeptide comprising an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410.

In some embodiments, the pharmaceutical composition or immunogenic composition comprises one or more viral vectors, each viral vector comprising one or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

In some embodiments, the pharmaceutical composition or immunogenic composition comprises a fusion polypeptide, a polynucleotide encoding such polypeptide or a vector expressing such fusion polypeptide, the fusion polypeptide comprising or consisting of the following polypeptide segments in sequential order, from N-terminus to C-terminus, optionally joined or connected by one or more linkers: SEQ ID NOs: 201, 78, 107, 96, 229, 172, 327, 6, 333, 243, 331, 192, 265, 311, 137, 15, 123, 30, 336, 302, 153, 219, 298, 121, 230, 240, 60, 241, 276, 113, 99, 21, 217 and 215; SEQ ID NOs: 78, 296, 1, 339, 197, 329, 232, 323, 303, 234, 90, 261, 274, 238, 211, 325, 137, 227, 209, 190, 341, 57, 225, 27, 210, 119, 19, 165, 334, 117, 153, 10, 97 and 300; or SEQ ID NOs: 296, 1, 78, 197, 339, 227, 261, 274, 238, 325, 137, 329, 303, 234, 90, 232, 27, 57, 225, 323, 190, 341, 119, 19, 165, 334, 117, 153, 10, 97 and 300. In some embodiments, the pharmaceutical composition or immunogenic composition comprises a fusion polypeptide, a polynucleotide encoding such polypeptide or a vector expressing such fusion polypeptide, the fusion polypeptide comprising or consisting of an amino acid sequence of any one of SEQ ID NOs: 367-377, 411, 422-424 and 431-435, or a sequence that is 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 367-377, 411, 422-424 and 431-435.

In various embodiments, the composition comprises one or more polynucleotides that encode one or more fusion proteins comprising an amino acid sequence of any one of any one of SEQ ID NOs: 345-377, 407-411, 422-424, and 430-435, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 345-377, 407-411, 422-424, and 430-435.

In various embodiments, the composition comprises two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

In various embodiments, the composition comprises a polynucleotide encoding a fusion protein that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of SEQ ID NO: 527. In some embodiments, the composition comprises a polynucleotide encoding a fusion protein that is at least 85% identical to the amino acid sequence of SEQ ID NO: 527. In some embodiments, the composition comprises a polynucleotide encoding a fusion protein that is at least 90% identical to the amino acid sequence of SEQ ID NO: 527. In some embodiments, the composition comprises a polynucleotide encoding a fusion protein that is at least 95% identical to the amino acid sequence of SEQ ID NO: 527. In some embodiments, the composition comprises a polynucleotide encoding a fusion protein that is at least 97% identical to the amino acid sequence of SEQ ID NO: 527. In some embodiments, the composition comprises a polynucleotide encoding a fusion protein that is at least 98% identical to the amino acid sequence of SEQ ID NO: 527. In some embodiments, the composition comprises a polynucleotide encoding a fusion protein that is at least 99% identical to the amino acid sequence of SEQ ID NO: 527. In some embodiments, the composition comprises a polynucleotide encoding a fusion protein that is at least 100% identical to the amino acid sequence of SEQ ID NO: 527. In some embodiments, the composition comprises a polynucleotide encoding a fusion protein that comprises 20, 19, 18, 17, 16, 15, 14, 13, 12, 11, 10, 9, 8, 7, 6, 5, 4, 3, 2, or 1 or fewer amino acid mismatches to the amino acid sequence of SEQ ID NO: 527. In some embodiments, the composition comprises a polynucleotide encoding a fusion protein that comprises 15 or fewer amino acid mismatches to the amino acid sequence of SEQ ID NO: 527. In some embodiments, the composition comprises a polynucleotide encoding a fusion protein that comprises 10 or fewer amino acid mismatches to the amino acid sequence of SEQ ID NO: 527. In some embodiments, the composition comprises a polynucleotide encoding a fusion protein that comprises 5 or fewer amino acid mismatches to the amino acid sequence of SEQ ID NO: 527. In some embodiments, the composition comprises a polynucleotide encoding a fusion protein that comprises 4 or fewer amino acid mismatches to the amino acid sequence of SEQ ID NO: 527. In some embodiments, the composition comprises a polynucleotide encoding a fusion protein that comprises 3 or fewer amino acid mismatches to the amino acid sequence of SEQ ID NO: 527. In some embodiments, the composition comprises a polynucleotide encoding a fusion protein that comprises 2 or fewer amino acid mismatches to the amino acid sequence of SEQ ID NO: 527. In some embodiments, the composition comprises a polynucleotide encoding a fusion protein that comprises 1 or fewer amino acid mismatches to the amino acid sequence of SEQ ID NO: 527.

In various embodiments, the composition comprises a polynucleotide encoding a fusion protein that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of SEQ ID NO: 528. In some embodiments, the composition comprises a polynucleotide encoding a fusion protein that is at least 85% identical to the amino acid sequence of SEQ ID NO: 528. In some embodiments, the composition comprises a polynucleotide encoding a fusion protein that is at least 90% identical to the amino acid sequence of SEQ ID NO: 528. In some embodiments, the composition comprises a polynucleotide encoding a fusion protein that is at least 95% identical to the amino acid sequence of SEQ ID NO: 528. In some embodiments, the composition comprises a polynucleotide encoding a fusion protein that is at least 97% identical to the amino acid sequence of SEQ ID NO: 528. In some embodiments, the composition comprises a polynucleotide encoding a fusion protein that is at least 98% identical to the amino acid sequence of SEQ ID NO: 528. In some embodiments, the composition comprises a polynucleotide encoding a fusion protein that is at least 99% identical to the amino acid sequence of SEQ ID NO: 528. In some embodiments, the composition comprises a polynucleotide encoding a fusion protein that is at least 100% identical to the amino acid sequence of SEQ ID NO: 528. In some embodiments, the composition comprises a polynucleotide encoding a fusion protein that comprises 20, 19, 18, 17, 16, 15, 14, 13, 12, 11, 10, 9, 8, 7, 6, 5, 4, 3, 2, or 1 or fewer amino acid mismatches to the amino acid sequence of SEQ ID NO: 528. In some embodiments, the composition comprises a polynucleotide encoding a fusion protein that comprises 15 or fewer amino acid mismatches to the amino acid sequence of SEQ ID NO: 528. In some embodiments, the composition comprises a polynucleotide encoding a fusion protein that comprises 10 or fewer amino acid mismatches to the amino acid sequence of SEQ ID NO: 528. In some embodiments, the composition comprises a polynucleotide encoding a fusion protein that comprises 5 or fewer amino acid mismatches to the amino acid sequence of SEQ ID NO: 528. In some embodiments, the composition comprises a polynucleotide encoding a fusion protein that comprises 4 or fewer amino acid mismatches to the amino acid sequence of SEQ ID NO: 528. In some embodiments, the composition comprises a polynucleotide encoding a fusion protein that comprises 3 or fewer amino acid mismatches to the amino acid sequence of SEQ ID NO: 528. In some embodiments, the composition comprises a polynucleotide encoding a fusion protein that comprises 2 or fewer amino acid mismatches to the amino acid sequence of SEQ ID NO: 528. In some embodiments, the composition comprises a polynucleotide encoding a fusion protein that comprises 1 or fewer amino acid mismatches to the amino acid sequence of SEQ ID NO: 528.

In various embodiments, the composition comprises one or more polynucleotides encoding one or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of any one of SEQ ID NOs: 345-371, 373-377, 407-411, 422-423, and 430-435.

In various embodiments, the composition comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 524. In some embodiments, the composition comprises a polynucleotide that is at least 85% identical to the nucleic acid sequence of SEQ ID NO: 524. In some embodiments, the composition comprises a polynucleotide that is at least 90% identical to the nucleic acid sequence of SEQ ID NO: 524. In some embodiments, the composition comprises a polynucleotide that is at least 95% identical to the nucleic acid sequence of SEQ ID NO: 524. In some embodiments, the composition comprises a polynucleotide that is at least 97% identical to the nucleic acid sequence of SEQ ID NO: 524. In some embodiments, the composition comprises a polynucleotide that is at least 98% identical to the nucleic acid sequence of SEQ ID NO: 524. In some embodiments, the composition comprises a polynucleotide that is at least 99% identical to the nucleic acid sequence of SEQ ID NO: 524. In some embodiments, the composition comprises a polynucleotide that is at least 100% identical to the nucleic acid sequence of SEQ ID NO: 524. In some embodiments, the composition comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 524. In some embodiments, the composition comprises a polynucleotide that comprises 15 or fewer nucleotide mismatches to the nucleic acid sequence of SEQ ID NO: 524. In some embodiments, the composition comprises a polynucleotide that comprises 10 or fewer nucleotide mismatches to the nucleic acid sequence of SEQ ID NO: 524. In some embodiments, the composition comprises a polynucleotide that comprises 5 or fewer nucleotide mismatches to the nucleic acid sequence of SEQ ID NO: 524. In some embodiments, the composition comprises a polynucleotide that comprises 4 or fewer nucleotide mismatches to the nucleic acid sequence of SEQ ID NO: 524. In some embodiments, the composition comprises a polynucleotide that comprises 3 or fewer nucleotide mismatches to the nucleic acid sequence of SEQ ID NO: 524. In some embodiments, the composition comprises a polynucleotide that comprises 2 or fewer nucleotide mismatches to the nucleic acid sequence of SEQ ID NO: 524. In some embodiments, the composition comprises a polynucleotide that comprises 1 or fewer nucleotide mismatches to the nucleic acid sequence of SEQ ID NO: 524. In some embodiments, the composition comprises a polynucleotide that comprises a codon-optimized nucleic acid sequence of SEQ ID NO: 524.

In various embodiments, the composition comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 525. In some embodiments, the composition comprises a polynucleotide that is at least 85% identical to the nucleic acid sequence of SEQ ID NO: 525. In some embodiments, the composition comprises a polynucleotide that is at least 90% identical to the nucleic acid sequence of SEQ ID NO: 525. In some embodiments, the composition comprises a polynucleotide that is at least 95% identical to the nucleic acid sequence of SEQ ID NO: 525. In some embodiments, the composition comprises a polynucleotide that is at least 97% identical to the nucleic acid sequence of SEQ ID NO: 525. In some embodiments, the composition comprises a polynucleotide that is at least 98% identical to the nucleic acid sequence of SEQ ID NO: 525. In some embodiments, the composition comprises a polynucleotide that is at least 99% identical to the nucleic acid sequence of SEQ ID NO: 525. In some embodiments, the composition comprises a polynucleotide that is at least 100% identical to the nucleic acid sequence of SEQ ID NO: 525. In some embodiments, the composition comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 525. In some embodiments, the composition comprises a polynucleotide that comprises 15 or fewer nucleotide mismatches to the nucleic acid sequence of SEQ ID NO: 525. In some embodiments, the composition comprises a polynucleotide that comprises 10 or fewer nucleotide mismatches to the nucleic acid sequence of SEQ ID NO: 525. In some embodiments, the composition comprises a polynucleotide that comprises 5 or fewer nucleotide mismatches to the nucleic acid sequence of SEQ ID NO: 525. In some embodiments, the composition comprises a polynucleotide that comprises 4 or fewer nucleotide mismatches to the nucleic acid sequence of SEQ ID NO: 525. In some embodiments, the composition comprises a polynucleotide that comprises 3 or fewer nucleotide mismatches to the nucleic acid sequence of SEQ ID NO: 525. In some embodiments, the composition comprises a polynucleotide that comprises 2 or fewer nucleotide mismatches to the nucleic acid sequence of SEQ ID NO: 525. In some embodiments, the composition comprises a polynucleotide that comprises 1 or fewer nucleotide mismatches to the nucleic acid sequence of SEQ ID NO: 525. In some embodiments, the composition comprises a polynucleotide that comprises a codon-optimized nucleic acid sequence of SEQ ID NO: 525.

In various embodiments, the composition comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 526. In some embodiments, the composition comprises a polynucleotide that is at least 85% identical to the nucleic acid sequence of SEQ ID NO: 526. In some embodiments, the composition comprises a polynucleotide that is at least 90% identical to the nucleic acid sequence of SEQ ID NO: 526. In some embodiments, the composition comprises a polynucleotide that is at least 95% identical to the nucleic acid sequence of SEQ ID NO: 526. In some embodiments, the composition comprises a polynucleotide that is at least 97% identical to the nucleic acid sequence of SEQ ID NO: 526. In some embodiments, the composition comprises a polynucleotide that is at least 98% identical to the nucleic acid sequence of SEQ ID NO: 526. In some embodiments, the composition comprises a polynucleotide that is at least 99% identical to the nucleic acid sequence of SEQ ID NO: 526. In some embodiments, the composition comprises a polynucleotide that is at least 100% identical to the nucleic acid sequence of SEQ ID NO: 526. In some embodiments, the composition comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 526. In some embodiments, the composition comprises a polynucleotide that comprises 15 or fewer nucleotide mismatches to the nucleic acid sequence of SEQ ID NO: 526. In some embodiments, the composition comprises a polynucleotide that comprises 10 or fewer nucleotide mismatches to the nucleic acid sequence of SEQ ID NO: 526. In some embodiments, the composition comprises a polynucleotide that comprises 5 or fewer nucleotide mismatches to the nucleic acid sequence of SEQ ID NO: 526. In some embodiments, the composition comprises a polynucleotide that comprises 4 or fewer nucleotide mismatches to the nucleic acid sequence of SEQ ID NO: 526. In some embodiments, the composition comprises a polynucleotide that comprises 3 or fewer nucleotide mismatches to the nucleic acid sequence of SEQ ID NO: 526. In some embodiments, the composition comprises a polynucleotide that comprises 2 or fewer nucleotide mismatches to the nucleic acid sequence of SEQ ID NO: 526. In some embodiments, the composition comprises a polynucleotide that comprises 1 or fewer nucleotide mismatches to the nucleic acid sequence of SEQ ID NO: 526. In some embodiments, the composition comprises a polynucleotide that comprises a codon-optimized nucleic acid sequence of SEQ ID NO: 526.

In various embodiments, the composition comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 520. In some embodiments, the composition comprises a polynucleotide that is at least 85% identical to the nucleic acid sequence of SEQ ID NO: 520. In some embodiments, the composition comprises a polynucleotide that is at least 90% identical to the nucleic acid sequence of SEQ ID NO: 520. In some embodiments, the composition comprises a polynucleotide that is at least 95% identical to the nucleic acid sequence of SEQ ID NO: 520. In some embodiments, the composition comprises a polynucleotide that is at least 97% identical to the nucleic acid sequence of SEQ ID NO: 520. In some embodiments, the composition comprises a polynucleotide that is at least 98% identical to the nucleic acid sequence of SEQ ID NO: 520. In some embodiments, the composition comprises a polynucleotide that is at least 99% identical to the nucleic acid sequence of SEQ ID NO: 520. In some embodiments, the composition comprises a polynucleotide that is at least 100% identical to the nucleic acid sequence of SEQ ID NO: 520. In some embodiments, the composition comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 520. In some embodiments, the composition comprises a polynucleotide that comprises 15 or fewer nucleotide mismatches to the nucleic acid sequence of SEQ ID NO: 520. In some embodiments, the composition comprises a polynucleotide that comprises 10 or fewer nucleotide mismatches to the nucleic acid sequence of SEQ ID NO: 520. In some embodiments, the composition comprises a polynucleotide that comprises 5 or fewer nucleotide mismatches to the nucleic acid sequence of SEQ ID NO: 520. In some embodiments, the composition comprises a polynucleotide that comprises 4 or fewer nucleotide mismatches to the nucleic acid sequence of SEQ ID NO: 520. In some embodiments, the composition comprises a polynucleotide that comprises 3 or fewer nucleotide mismatches to the nucleic acid sequence of SEQ ID NO: 520. In some embodiments, the composition comprises a polynucleotide that comprises 2 or fewer nucleotide mismatches to the nucleic acid sequence of SEQ ID NO: 520. In some embodiments, the composition comprises a polynucleotide that comprises 1 or fewer nucleotide mismatches to the nucleic acid sequence of SEQ ID NO: 520. In some embodiments, the composition comprises a polynucleotide that comprises a codon-optimized nucleic acid sequence of SEQ ID NO: 520.

In various embodiments, the composition comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 521. In some embodiments, the composition comprises a polynucleotide that is at least 85% identical to the nucleic acid sequence of SEQ ID NO: 521. In some embodiments, the composition comprises a polynucleotide that is at least 90% identical to the nucleic acid sequence of SEQ ID NO: 521. In some embodiments, the composition comprises a polynucleotide that is at least 95% identical to the nucleic acid sequence of SEQ ID NO: 521. In some embodiments, the composition comprises a polynucleotide that is at least 97% identical to the nucleic acid sequence of SEQ ID NO: 521. In some embodiments, the composition comprises a polynucleotide that is at least 98% identical to the nucleic acid sequence of SEQ ID NO: 521. In some embodiments, the composition comprises a polynucleotide that is at least 99% identical to the nucleic acid sequence of SEQ ID NO: 521. In some embodiments, the composition comprises a polynucleotide that is at least 100% identical to the nucleic acid sequence of SEQ ID NO: 521. In some embodiments, the composition comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 521. In some embodiments, the composition comprises a polynucleotide that comprises 15 or fewer nucleotide mismatches to the nucleic acid sequence of SEQ ID NO: 521. In some embodiments, the composition comprises a polynucleotide that comprises 10 or fewer nucleotide mismatches to the nucleic acid sequence of SEQ ID NO: 521. In some embodiments, the composition comprises a polynucleotide that comprises 5 or fewer nucleotide mismatches to the nucleic acid sequence of SEQ ID NO: 521. In some embodiments, the composition comprises a polynucleotide that comprises 4 or fewer nucleotide mismatches to the nucleic acid sequence of SEQ ID NO: 521. In some embodiments, the composition comprises a polynucleotide that comprises 3 or fewer nucleotide mismatches to the nucleic acid sequence of SEQ ID NO: 521. In some embodiments, the composition comprises a polynucleotide that comprises 2 or fewer nucleotide mismatches to the nucleic acid sequence of SEQ ID NO: 521. In some embodiments, the composition comprises a polynucleotide that comprises 1 or fewer nucleotide mismatches to the nucleic acid sequence of SEQ ID NO: 521. In some embodiments, the composition comprises a polynucleotide that comprises a codon-optimized nucleic acid sequence of SEQ ID NO: 521.

In various embodiments, the composition comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 522. In some embodiments, the composition comprises a polynucleotide that is at least 85% identical to the nucleic acid sequence of SEQ ID NO: 522. In some embodiments, the composition comprises a polynucleotide that is at least 90% identical to the nucleic acid sequence of SEQ ID NO: 522. In some embodiments, the composition comprises a polynucleotide that is at least 95% identical to the nucleic acid sequence of SEQ ID NO: 522. In some embodiments, the composition comprises a polynucleotide that is at least 97% identical to the nucleic acid sequence of SEQ ID NO: 522. In some embodiments, the composition comprises a polynucleotide that is at least 98% identical to the nucleic acid sequence of SEQ ID NO: 522. In some embodiments, the composition comprises a polynucleotide that is at least 99% identical to the nucleic acid sequence of SEQ ID NO: 522. In some embodiments, the composition comprises a polynucleotide that is at least 100% identical to the nucleic acid sequence of SEQ ID NO: 522. In some embodiments, the composition comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 522. In some embodiments, the composition comprises a polynucleotide that comprises 15 or fewer nucleotide mismatches to the nucleic acid sequence of SEQ ID NO: 522. In some embodiments, the composition comprises a polynucleotide that comprises 10 or fewer nucleotide mismatches to the nucleic acid sequence of SEQ ID NO: 522. In some embodiments, the composition comprises a polynucleotide that comprises 5 or fewer nucleotide mismatches to the nucleic acid sequence of SEQ ID NO: 522. In some embodiments, the composition comprises a polynucleotide that comprises 4 or fewer nucleotide mismatches to the nucleic acid sequence of SEQ ID NO: 522. In some embodiments, the composition comprises a polynucleotide that comprises 3 or fewer nucleotide mismatches to the nucleic acid sequence of SEQ ID NO: 522. In some embodiments, the composition comprises a polynucleotide that comprises 2 or fewer nucleotide mismatches to the nucleic acid sequence of SEQ ID NO: 522. In some embodiments, the composition comprises a polynucleotide that comprises 1 or fewer nucleotide mismatches to the nucleic acid sequence of SEQ ID NO: 522. In some embodiments, the composition comprises a polynucleotide that comprises a codon-optimized nucleic acid sequence of SEQ ID NO: 522.

In various embodiments, the composition comprises a polynucleotide that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NO: 523. In some embodiments, the composition comprises a polynucleotide that is at least 85% identical to the nucleic acid sequence of SEQ ID NO: 523. In some embodiments, the composition comprises a polynucleotide that is at least 90% identical to the nucleic acid sequence of SEQ ID NO: 523. In some embodiments, the composition comprises a polynucleotide that is at least 95% identical to the nucleic acid sequence of SEQ ID NO: 523. In some embodiments, the composition comprises a polynucleotide that is at least 97% identical to the nucleic acid sequence of SEQ ID NO: 523. In some embodiments, the composition comprises a polynucleotide that is at least 98% identical to the nucleic acid sequence of SEQ ID NO: 523. In some embodiments, the composition comprises a polynucleotide that is at least 99% identical to the nucleic acid sequence of SEQ ID NO: 523. In some embodiments, the composition comprises a polynucleotide that is at least 100% identical to the nucleic acid sequence of SEQ ID NO: 523. In some embodiments, the composition comprises a polynucleotide that comprises 20, 19, 18, 17, 16, 15, 14, 13, 12, 11, 10, 9, 8, 7, 6, 5, 4, 3, 2, or 1 or fewer nucleotide mismatches to the nucleic acid sequence of SEQ ID NO: 523. In some embodiments, the composition comprises a polynucleotide that comprises 15 or fewer nucleotide mismatches to the nucleic acid sequence of SEQ ID NO: 523. In some embodiments, the composition comprises a polynucleotide that comprises 10 or fewer nucleotide mismatches to the nucleic acid sequence of SEQ ID NO: 523. In some embodiments, the composition comprises a polynucleotide that comprises 5 or fewer nucleotide mismatches to the nucleic acid sequence of SEQ ID NO: 523. In some embodiments, the composition comprises a polynucleotide that comprises 4 or fewer nucleotide mismatches to the nucleic acid sequence of SEQ ID NO: 523. In some embodiments, the composition comprises a polynucleotide that comprises 3 or fewer nucleotide mismatches to the nucleic acid sequence of SEQ ID NO: 523. In some embodiments, the composition comprises a polynucleotide that comprises 2 or fewer nucleotide mismatches to the nucleic acid sequence of SEQ ID NO: 523. In some embodiments, the composition comprises a polynucleotide that comprises 1 or fewer nucleotide mismatches to the nucleic acid sequence of SEQ ID NO: 523. In some embodiments, the composition comprises a polynucleotide that comprises a codon-optimized nucleic acid sequence of SEQ ID NO: 523.

### Methods of Treatment

Further provided are methods for treating or preventing an HIV infection or a related disease or disorder in a subject in need thereof (e.g., a human subject), comprising providing to a subject in need thereof an effective amount of one or more fusion polypeptides, as described herein, or one or more polynucleotides encoding one or more fusion polypeptides, as described herein, or an expression vector, viral expression vector, viral vector, SAM, LNP, PNP or nanoemulsion comprising such polynucleotides, as described herein. As used herein, the term “subject” refers to a mammal. The mammal can be any mammal, for example, a human, a non-human primate (e.g., a macaque), a rodent (e.g., mouse, rat, guinea pig), a dog, a cat, or a domesticated animal such as a cow, a horse, a goat, a camel, a sheep or a pig. The term “patient” refers to a human subject. As used herein, the term “effective amount” in the context of the administration of a therapy to a subject refers to the amount of a therapy that achieves a desired prophylactic or therapeutic effect. The polynucleotide may be present in a vector, e.g., a viral vector, as described herein. In some embodiments, the related disease or disorder is caused by infection with HIV. In other embodiments, it is acquired immune deficiency syndrome (AIDS). In certain embodiments, the subject is a virologically suppressed HIV-infected mammal, while in other embodiments, the subject is a treatment-naïve HIV-infected mammal or a treatment experienced HIV-infected subject that is not virologically suppressed. In certain embodiments, a treatment-naïve subject has a viral load between <50 copies/mL and 10⁸ copies/ml. In certain embodiments, a virologically suppressed subject has a viral load <50 copies/ml. In another embodiment, the subject is a mammal, e.g., a human. In certain embodiments, the subject has been diagnosed with an HIV, e.g., HIV-1 or HIV-2, infection or a related disease or disorder, e.g., AIDS, or is considered at risk for developing an HIV, e.g., HIV-1 or HIV-2, infection or a related disease or disorder, e.g., AIDS. Subjects at risk for HIV-related diseases or disorders include patients who have come into contact with an infected person or who have been exposed to HIV in some other way. Administration of a prophylactic agent can occur prior to the manifestation of symptoms characteristic of HIV-related disease or disorder, such that a disease or disorder is prevented or, alternatively, delayed in its progression.

In some embodiments, the subject is chronically infected with HIV-1. In some embodiments, the subject is acutely infected with HIV-1, e.g., has an HIV-1 infection of Fiebig stage IV or earlier, e.g. Fiebig stage III, Fiebig stage II or Fiebig stage I. In some embodiments, the subject is not receiving antiretroviral therapy (ART) or ART is discontinued prior to administration of the one or more compositions. In some embodiments, ART is discontinued after one or more administrations of the compositions. In some embodiments, ART is administered concurrently with administration of one or more fusion polypeptides, as described herein, or one or more polynucleotides encoding one or more fusion polypeptides, as described herein, or one or more vectors expressing one or more fusion polypeptides, as described herein.

Also provided are methods for preventing or inhibiting an increase in HIV virus titer, virus replication, virus proliferation or an amount of an HIV viral DNA, HIV proviral DNA, or HIV viral protein in a subject (e.g., a human subject). In one embodiment, the method comprises providing to the subject in need thereof an amount of an one or more fusion polypeptides, as described herein, or one or more polynucleotides encoding one or more fusion polypeptides, as described herein, or an expression vector, viral expression vector, viral vector, SAM, LNP, PNP or nanoemulsion comprising such polynucleotides, as described herein, effective to prevent an increase in HIV titer, virus replication, or an amount of an HIV protein of one or more HIV strains or isolates in the subject. In certain embodiments, the method further comprises measuring an amount of HIV viral or proviral DNA or protein at one or more time points, e.g., before and after the subject in provided with one or more fusion polypeptides, as described herein, or one or more polynucleotides encoding one or more fusion polypeptides, as described herein, or one or more vectors expressing one or more fusion polypeptides, as described herein. Methods and biomarkers for determining an amount of HIV viral or proviral DNA or protein in a subject are known and available in the art, and described for example, in Siliciano, J. D. et al., Curr Opin. HIV AIDS, 5 (6): 491-7 (2010), and Rouzioux, C. et al., Curr Opin HIV AIDS, 8 (3): 170-5 (2013).

In some embodiments, one or more fusion polypeptides, as described herein, or one or more polynucleotides encoding one or more fusion polypeptides, as described herein, or an expression vector, viral expression vector, viral vector, SAM, LNP, PNP or nanoemulsion comprising such polynucleotides, as described herein, may be used in, for example, methods of inhibiting certain viruses such as HIV isolates described herein, prophylactic inhibiting or preventing infections of certain viruses such as HIV isolates described herein, detection of certain viruses such as HIV isolates described herein in a sample, inhibiting certain viruses such as HIV isolates described herein, or diagnosis of certain viruses such as HIV isolates described herein.

For in vivo treatment of mammalian subject, e.g., humans, the subject may be administered or provided a pharmaceutical composition comprising one or more fusion polypeptides, as described herein, or one or more polynucleotides encoding one or more fusion polypeptides, as described herein, or one or more vectors expressing one or more fusion polypeptides, as described herein. When used for in vivo therapy, the one or more fusion polypeptides, as described herein, or one or more polynucleotides encoding one or more fusion polypeptides, as described herein, or one or more vectors expressing one or more fusion polypeptides, as described herein, are typically administered or provided to the patient in therapeutically effective amounts (i.e., amounts that eliminate or reduce the patient's viral burden and/or viral reservoir). The one or more fusion polypeptides, as described herein, or one or more polynucleotides encoding one or more fusion polypeptides, as described herein, or one or more vectors expressing one or more fusion polypeptides, as described herein, are administered or provided to a mammalian subject, e.g., a human, in accord with known methods, such as, but not limited to, intravenous administration, e.g., as a bolus or by continuous infusion over a period of time, by intramuscular, intraperitoneal, intracerebrospinal, subcutaneous, intraarticular, intrasynovial, intrathecal, oral, topical, or inhalation routes. The one or more fusion polypeptides, as described herein, or one or more polynucleotides encoding one or more fusion polypeptides, as described herein, or one or more vectors expressing one or more fusion polypeptides, as described herein, may be administered parenterally, when possible, at the target cell site, or intravenously. In one embodiment, administration of the one or more fusion polypeptides, as described herein, or one or more polynucleotides encoding one or more fusion polypeptides, as described herein, or one or more vectors expressing one or more fusion polypeptides, as described herein, to the subject is via an intravenous route. In another embodiment, administration of the one or more fusion polypeptides, as described herein, or one or more polynucleotides encoding one or more fusion polypeptides, as described herein, or one or more vectors expressing one or more fusion polypeptides, as described herein, to the subject is via a subcutaneous route. In additional embodiments, pharmaceutical compositions of the disclosure are administered to a subject systemically, parenterally, or locally (e.g., mucosally, including buccal, intrarectal and/or intravaginal routes).

In certain embodiments, the present disclosure provides a method for treating an HIV infection, comprising administering to a human subject in need thereof a therapeutically effective amount of one or more fusion polypeptides, as described herein, or one or more polynucleotides encoding one or more fusion polypeptides, as described herein, or one or more vectors expressing one or more fusion polypeptides, as described herein. In some embodiments, the present disclosure provides a method for preventing an HIV infection, comprising administering to a human subject in need thereof a therapeutically effective amount of one or more fusion polypeptides, as described herein, or one or more polynucleotides encoding one or more fusion polypeptides, as described herein, or an expression vector, viral expression vector, viral vector, SAM, LNP, PNP or nanoemulsion comprising such polynucleotides, as described herein.

In various embodiments, the methods comprise administering a single fusion polypeptide, or a polynucleotide encoding the fusion polypeptide, or an expression vector, viral expression vector, viral vector, SAM, LNP, PNP or nanoemulsion comprising such polynucleotides, wherein the fusion polypeptide comprises two or more multivalent polypeptide segments, e.g., bivalent polypeptide segments. In some embodiments, two or more fusion polypeptides, or two or more viral expression vectors encoding the fusion polypeptides, are administered to the subject simultaneously or concurrently. In some embodiments, the two or more fusion polypeptides, or two or more polynucleotides or two or more viral expression vectors encoding the fusion polypeptides, are in the form of a bivalent antigen composition.

In some embodiments, the methods entail administering to the subject: (1) one or more fusion polypeptides, or polynucleotides encoding, or viral expression vectors expressing the fusion polypeptides, the fusion polypeptides comprising or consisting of the following polypeptide segments in sequential order, from N-terminus to C-terminus, optionally joined or connected by one or more linkers: SEQ ID NOs: 70, 76, 94, 151 and 161; or SEQ ID NOs: 71, 77, 95, 152 and 162; and (2) one or more fusion polypeptides, or polynucleotides encoding, or viral expression vectors expressing the fusion polypeptides, the fusion polypeptides comprising or consisting of the following polypeptide segments in sequential order, from N-terminus to C-terminus, optionally joined or connected by one or more linkers: SEQ ID NOs: 188, 305, 28, 41, 294, 4, 176, 11, 319, 259, 282, 223, 213 and 37; SEQ ID NOs: 188, 305, 28, 41 and 294; SEQ ID NOS: 4, 176, 11, 319, 259, 282, 223, 213 and 37; SEQ ID NOs: 189, 306, 29, 42, 295, 5, 177, 12, 320, 260, 283, 224, 214 and 38; SEQ ID NOs: 189, 306, 29, 42 and 295; SEQ ID NOs: 5, 177, 12, 320, 260, 283, 224, 214 and 38; SEQ ID NOs: 305, 319, 259, 282, 223, 213, 294, 176 and 188; SEQ ID NOs: 306, 320, 260, 283, 224, 214, 295, 177 and 189; SEQ ID NOs: 305, 294, 223, 213, 176, 259, 319, 188 and 282; SEQ ID NOs: 306, 295, 224, 214, 177, 260, 320, 189 and 283; SEQ ID NOs: 305, 294, 319, 259, 282, 223, 176, and 188; SEQ ID NOs: 306, 295, 320, 260, 283, 224, 177 and 189; SEQ ID NOs: 305, 223, 294, 176, 259, 319, 188 and 282; or SEQ ID NOs: 306, 224, 295, 177, 260, 320, 189 and 283.

In some embodiments, the methods entail administering to the subject: (1) one or more fusion polypeptides, or polynucleotides encoding, or viral expression vectors expressing the fusion polypeptides, the fusion polypeptides comprising or consisting of an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430; and (2) one or more fusion polypeptides, or polynucleotides encoding, or viral expression vectors expressing the fusion polypeptides, the fusion polypeptides comprising or consisting of an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410.

In some embodiments, the method comprises administering to the subject one or more viral vectors, wherein each viral vector comprises two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

In some embodiments, the methods entail administering to the subject one or more fusion polypeptides, or polynucleotides encoding the fusion polypeptides, the fusion polypeptides comprising or consisting of the following polypeptide segments in sequential order, from N-terminus to C-terminus, optionally joined or connected by one or more linkers: SEQ ID NOs: 201, 78, 107, 96, 229, 172, 327, 6, 333, 243, 331, 192, 265, 311, 137, 15, 123, 30, 336, 302, 153, 219, 298, 121, 230, 240, 60, 241, 276, 113, 99, 21, 217 and 215; SEQ ID NOs: 78, 296, 1, 339, 197, 329, 232, 323, 303, 234, 90, 261, 274, 238, 211, 325, 137, 227, 209, 190, 341, 57, 225, 27, 210, 119, 19, 165, 334, 117, 153, 10, 97 and 300; or SEQ ID NOs: 296, 1, 78, 197, 339, 227, 261, 274, 238, 325, 137, 329, 303, 234, 90, 232, 27, 57, 225, 323, 190, 341, 119, 19, 165, 334, 117, 153, 10, 97 and 300, or an expression vector, viral expression vector, viral vector, SAM, LNP, PNP or nanoemulsion comprising such polynucleotides.

In some embodiments, the methods entail administering to the subject one or more fusion polypeptides, or polynucleotides encoding the fusion polypeptides, the fusion polypeptides comprising or consisting of an amino acid sequence of any one of SEQ ID NOs: 367-377, 411, 422-424 and 431-435, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 367-377, 411, 422-424 and 431-435, or an expression vector, viral expression vector, viral vector, SAM, LNP, PNP or nanoemulsion comprising such polynucleotides.

In some embodiments, the methods entail administering to the subject one or more polynucleotides comprising or consisting of a nucleic acid sequence of any one of SEQ ID NOS: 520-521, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 520-521, or an expression vector, viral expression vector, viral vector, SAM, LNP, PNP or nanoemulsion comprising such polynucleotides.

In some embodiments, the methods entail administering to the one or more polynucleotides comprising or consisting of a nucleic acid sequence of any one of SEQ ID NOS: 522-523, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOS: 522-523, or an expression vector, viral expression vector, viral vector, SAM, LNP, PNP or nanoemulsion comprising such polynucleotides.

In some embodiments, the methods entail administering to the subject one or more polynucleotides comprising or consisting of a nucleic acid sequence of any one of SEQ ID NOS: 524-526, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 524-526, or an expression vector, viral expression vector, viral vector, SAM, LNP, PNP or nanoemulsion comprising such polynucleotides.

In some embodiments, the methods entail administering to the subject one or more fusion polypeptides, or polynucleotides encoding such polypeptides, the fusion polypeptides comprising or consisting of an amino acid sequence of any one of SEQ ID NOs: 527-528, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 527-528, or an expression vector, viral expression vector, viral vector, SAM, LNP, PNP or nanoemulsion comprising such polynucleotides.

In some embodiments, the methods entail administering to the subject one or more viral expression vectors or viral vectors that express one or more of the fusion polypeptides or comprise the polynucleotides described herein. In various embodiments, the methods entail administering from about 10³ to about 10¹² viral focus forming units (FFU) or plaque forming units (PFU) or infectious units (IU) or viral particles (vp), e.g. from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration.

In some embodiments, the methods entail administering to the subject one or more SAMs that express one or more of the fusion polypeptides or comprise or consists of the polynucleotides described herein, or LNPs, PNPs, or nanoemulsions comprising such SAMS. In various embodiments, the methods entail administering from about 1 μg to about 100, 200, 300, 400, 500, 600, 700, 800, 900, or 1000 μg, per administration.

In various embodiments, the methods implement a prime-boost regimen. In various embodiments, the prime-boost regimen comprises administering a priming composition at a first time point and administering one or more boosting compositions at one or more subsequent time points (e.g., prime-boost-boost-boost, etc.). In various embodiments, the prime-boost regimen comprises one or more iterations of administering a priming composition at a first time point and administering a boosting composition at a second time point (e.g., prime-boost-prime-boost, etc.). Implementing a prime-boost regimen comprises one or more iterations of administering a priming composition at a first time point and administering a boosting composition at a second time point (e.g., prime-boost-prime-boost, etc.) can facilitate an immune response predominantly focused or trained on the fusion polypeptides, and reduce or avoid inducing an immune response focused or trained on the vector backbone and/or vector specific proteins. In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise the same immunogenic composition. In some embodiments, the priming composition and the boosting composition comprise different immunogenic compositions. In some embodiments, the priming composition and the boosting composition comprise the same one or more fusion polypeptides and same polynucleotide or viral expression vector. In some embodiments, the priming composition and the boosting composition comprise different fusion polypeptides and the same polynucleotide or viral expression vectors. In some embodiments, the priming composition and the boosting composition comprise the same fusion polypeptides and different polynucleotide or viral expression vectors. In some embodiments, the methods entail priming with a first polynucleotide or viral expression vector, and boosting with a second polynucleotide or viral expression vector.

In various embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM.

In some embodiments, after one or more fusion polypeptides, or polynucleotides encoding or SAMs or vectors expressing such fusion polypeptides, or viral vectors, LNPs, PNPs, or nanoemulsions comprising such polynucleotides, as described herein, optionally with one or more additional therapeutic agents, described herein, the subject does not exhibit symptoms of HIV or AIDS in the absence of anti-retroviral treatment (ART) for at least 6 months, at least 1 year, at least 2 years, at least 3 years, or more. In some embodiments, after one or more administrations of the binding molecule, the subject has a viral load of copies/ml blood of less than 500, e.g., less than 400, less than 300, less than 200, less than 100, less than 50, in the absence of anti-retroviral treatment (ART) for at least 6 months, at least 1 year, at least 2 years, at least 3 years, or more.

### Combination Therapies

In certain embodiments, a method for treating or preventing an HIV infection in a human having or at risk of having the infection is provided, comprising administering to the human a therapeutically effective amount of one or more fusion polypeptides, or polynucleotides encoding or SAMs or vectors expressing such fusion polypeptides, or viral vectors, LNPs, PNPs, or nanoemulsions comprising such polynucleotides, as disclosed herein, in combination with a therapeutically effective amount of one or more (e.g., one, two, three, one or two, or one to three) additional therapeutic agents. In one embodiment, a method for treating an HIV infection in a human having or at risk of having the infection is provided, comprising administering to the human a therapeutically effective amount of a compound disclosed herein, or a pharmaceutically acceptable salt thereof, in combination with a therapeutically effective amount of one or more (e.g., one, two, three, one or two, or one to three) additional therapeutic agents.

In various embodiments, one or more fusion polypeptides, or polynucleotides encoding or SAMs or vectors expressing such fusion polypeptides, or viral vectors, LNPs, PNPs, or nanoemulsions comprising such polynucleotides, as disclosed herein, are administered in combination with one or more (e.g., one, two, three, one or two, or one to three) additional therapeutic agents.

In certain embodiments, the provided are methods for treating an HIV infection, comprising administering to a patient in need thereof a therapeutically effective amount of a compound disclosed herein, or a pharmaceutically acceptable salt thereof, in combination with a therapeutically effective amount of one or more (e.g., one, two, three, one or two, or one to three) additional therapeutic agents which are suitable for treating an HIV infection.

In certain embodiments, one or more fusion polypeptides, or polynucleotides encoding or vectors expressing such fusion polypeptides, as disclosed herein, is co-formulated with one, two, three, four, or more additional therapeutic agents, and a pharmaceutically acceptable carrier. In certain embodiments, one or more fusion polypeptides, or polynucleotides encoding or vectors expressing such fusion polypeptides, as disclosed herein, or a pharmaceutically acceptable salt thereof, is combined with two additional therapeutic agents. As appropriate, the one, two, three, four, or more additional therapeutic agents can be different therapeutic agents selected from the same class of therapeutic agents, and/or they can be selected from different classes of therapeutic agents.

### Administration of HIV Combination Therapy

In certain embodiments, a one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are administered with one or more additional therapeutic agents. Co-administration of a compound disclosed herein with one or more additional therapeutic agents generally refers to simultaneous or concurrent, or sequential, administration of a compound disclosed herein and one or more additional therapeutic agents, such that therapeutically effective amounts of the compound disclosed herein and the one or more additional therapeutic agents are both present in the body of the patient. When administered sequentially, the combination may be administered in two or more administrations.

Co-administration includes administration of unit dosages of the compounds disclosed herein before or after administration of unit dosages of one or more additional therapeutic agents. For example, the one or more fusion polypeptides, or polynucleotides encoding or vectors expressing such fusion polypeptides, as disclosed herein, may be administered within seconds, minutes, or hours of the administration of the one or more additional therapeutic agents. In some embodiments, a unit dose of a one or more fusion polypeptides, or polynucleotides encoding or vectors expressing such fusion polypeptides, as disclosed herein, is administered first, followed within seconds or minutes by administration of a unit dose of one or more additional therapeutic agents. Alternatively, a unit dose of one or more additional therapeutic agents is administered first, followed by administration of a unit dose of a one or more fusion polypeptides, or polynucleotides encoding or vectors expressing such fusion polypeptides, as disclosed herein, within seconds or minutes. In other embodiments, a unit dose of one or more fusion polypeptides, or polynucleotides encoding or vectors expressing such fusion polypeptides, as disclosed herein, is administered first, followed, after a period of hours (e.g., 1-12 hours), by administration of a unit dose of one or more additional therapeutic agents. In yet other embodiments, a unit dose of one or more additional therapeutic agents is administered first, followed, after a period of hours (e.g., 1-12 hours), by administration of a unit dose of one or more fusion polypeptides, or polynucleotides encoding or vectors expressing such fusion polypeptides, as disclosed herein.

In certain embodiments, one or more fusion polypeptides, or polynucleotides encoding such fusion polypeptides, or an expression vector, viral expression vector, viral vector, SAM, LNP, PNP or nanoemulsion comprising such polynucleotides, as disclosed herein, is combined with one or more additional therapeutic agents in a unitary dosage form for simultaneous or concurrent administration to a patient, for example as an aqueous formulation for intravenous, intramuscular, intradermal or subcutaneous administration. In certain embodiments, one or more fusion polypeptides, or polynucleotides encoding or vectors expressing such fusion polypeptides, as disclosed herein, is combined with one or more additional therapeutic agents in a unitary dosage form for simultaneous or concurrent administration to a patient, for example as an intrarectal suppository.

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, can be co-formulated or co-administered with one or more other compounds useful for treating HIV. In certain embodiments, the co-formulation or co-administration can comprise another active agent for treating HIV, such as an anti-HIV antibody, a toll-like receptor (TLR) agonist, an immune checkpoint inhibitor, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments, the one or more active agents are suitable for once daily dosing, weekly dosing, monthly dosing, every 3 months dosing, every four months dosing, bi-annual dosing, or annual dosing, as appropriate.

In some embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, and the one or more additional therapeutic agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, HIV Tat or Rev inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, HIV-1 Nef modulators, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

### Combination Drugs

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with an HIV combination drug. Examples of combination drugs that can be employed with an agent of this disclosure include ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY (bictegravir+emtricitabine+tenofovir alafenamide), DOVATO, TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine; cabotegravir+rilpivirine; elpida (elsulfavirine; VM-1500; VM-1500A).

Examples of other drugs for treating HIV that can be combined with the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, include acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, ABX-464, AG-1105, APH-0812, BIT-225, CYT-107, HGTV-43, HPH-116, HS-10234, IMO-3100, IND-02, MK-1376, MK-2048, MK-4250, MK-8507, MK-8591 NOV-205, PA-1050040 (PA-040), PGN-007, SCY-635, SB-9200, SCB-719, TR-452, TEV-90110, TEV-90112, TEV-90111, TEV-90113, RN-18, Immuglo, and VIR-576.

### HIV Protease Inhibitors

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with an HIV protease inhibitor. Examples of HIV protease inhibitors that can be combined with an agent of this disclosure include amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, MK-8122, TMB-607, and TMC-310911.

### HIV Reverse Transcriptase Inhibitors

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a non-nucleoside or non-nucleotide inhibitor. Examples of HIV non-nucleoside or non-nucleotide inhibitors of reverse transcriptase that can be combined with an agent of this disclosure include dapivirine, delavirdine, delavirdine mesylate, doravirine, efavirenz, etravirine, lentinan, nevirapine, rilpivirine, ACC-007, AIC-292, KM-023, PC-1005, and elsulfavirine (VM-1500).

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with an HIV nucleoside or nucleotide inhibitor. Examples of HIV nucleoside or nucleotide inhibitors of reverse transcriptase that can be combined with an agent of this disclosure include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, MK-8591, MK-858, VM-2500 and KP-1461.

### HIV Integrase Inhibitors

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with an HIV integrase inhibitor. Examples of HIV integrase inhibitors that can be combined with an agent of this disclosure include elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, dolutegravir, JTK-351, bictegravir, AVX-15567, cabotegravir (long-acting injectable), diketo quinolin-4-1 derivatives, integrase-LEDGF inhibitor, ledgins, M-522, M-532, NSC-310217, NSC-371056, NSC-48240, NSC-642710, NSC-699171, NSC-699172, NSC-699173, NSC-699174, stilbenedisulfonic acid, T 169, VM-3500 and cabotegravir.

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a HIV non-catalytic site, or allosteric, integrase inhibitor (NCINI). Examples of HIV non-catalytic site, or allosteric, integrase inhibitors (NCINI) that can be combined with an agent of this disclosure include CX-05045, CX-05168, and CX-14442.

### HIV Entry Inhibitors

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with an HIV entry inhibitor. Examples of HIV entry (fusion) inhibitors that can be combined with an agent of this disclosure include cenicriviroc, CCR5 inhibitors, gp41 inhibitors, CD4 attachment inhibitors, gp120 inhibitors, and CXCR4 inhibitors.

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a CCR5 inhibitor. Examples of CCR5 inhibitors that can be combined with an agent of this disclosure include aplaviroc, vicriviroc, maraviroc, cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, and vMIP (Haimipu).

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a gp41 inhibitor. Examples of gp41 inhibitors that can be combined with an agent of this disclosure include albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a CD4 attachment inhibitor. Examples of CD4 attachment inhibitors that can be combined with an agent of this disclosure include ibalizumab and CADA analogs.

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a gp120 inhibitor. Examples of gp120 inhibitors that can be combined with an agent of this disclosure include Radha-108 (receptol) 3B3-PE38, BanLec, bentonite-based nanomedicine, fostemsavir tromethamine, IQP-0831, and BMS-663068.

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a CXCR4 inhibitor. Examples of CXCR4 inhibitors that can be combined with an agent of this disclosure include plerixafor, ALT-1188, N15 peptide, and vMIP (Haimipu).

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a HIV maturation inhibitor. Examples of HIV maturation inhibitors that can be combined with an agent of this disclosure include BMS-955176, GSK-3640254 and GSK-2838232.

### Latency Reversing Agents

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a latency reversing agent (LRA). Examples of latency reversing agents that can be combined with an agent of this disclosure include toll-like receptor (TLR) agonists (including TLR7 agonists, e.g., GS-9620), histone deacetylase (HDAC) inhibitors, proteasome inhibitors such as velcade, protein kinase C (PKC) activators, Smyd2 inhibitors, BET-bromodomain 4 (BRD4) inhibitors, ionomycin, IAP antagonists (inhibitor of apoptosis proteins, such as APG-1387, LBW-242), Second mitochondria-derived activator of caspases (SMAC; NCBI Gene ID: 56616) mimetics (including ciapavir, BI-891065, TL32711, LCL161, GDC-0917, HGS1029, AT-406), PMA, SAHA (suberanilohydroxamic acid, or suberoyl, anilide, and hydroxamic acid), NIZ-985, IL-15 modulating antibodies (including IL-15, IL-15 fusion proteins and IL-15 receptor agonists), JQ1, disulfiram, amphotericin B, and ubiquitin inhibitors such as largazole analogs, APH-0812, and GSK-343. Examples of PKC activators include indolactam, prostratin, ingenol B, and DAG-lactones.

### Histone Deacetylase (HDAC) Inhibitors

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CUDC-907 (fimepinostat), entinostat, givinostat, mocetinostat, panobinostat, pracinostat, quisinostat (JNJ-26481585), resminostat, ricolinostat, romidepsin, SHP-141, valproic acid (VAL-001), vorinostat, tinostamustine, remetinostat, entinostat.

### Capsid Inhibitor

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a capsid inhibitor. Examples of capsid inhibitors that can be combined with an agent of this disclosure include capsid polymerization inhibitors or capsid disrupting compounds, HIV nucleocapsid p7 (NCp7) inhibitors such as azodicarbonamide, HIV p24 capsid protein inhibitors, GS-6207 (lenacapavir), GS-CA1, AVI-621, AVI-101, AVI-201, AVI-301, and AVI-CAN1-15 series, and compounds described in this patent (GSK WO2019/087016).

### Immune Checkpoint Modulators

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with one or more blockers, antagonists or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu, et al., J Exp Clin Cancer Res. (2018) 37:110). In various embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18 (11): 671-688).

Examples of immune checkpoint proteins or receptors include without limitation CD27 (NCBI Gene ID: 939), CD70 (NCBI Gene ID: 970), CD40 (NCBI Gene ID: 958), CD40LG (NCBI Gene ID: 959), CD47 (NCBI Gene ID: 961), CD48 (SLAMF2; NCBI Gene ID: 962), transmembrane and immunoglobulin domain containing 2 (TMIGD2, CD28H; NCBI Gene ID: 126259), CD84 (LY9B, SLAMF5; NCBI Gene ID: 8832), CD96 (NCBI Gene ID: 10225), CD160 (NCBI Gene ID: 11126), MS4A1 (CD20; NCBI Gene ID: 931), CD244 (SLAMF4; NCBI Gene ID: 51744); CD276 (B7H3; NCBI Gene ID: 80381); V-set domain containing T cell activation inhibitor 1 (VTCN1, B7H4; NCBI Gene ID: 79679); V-set immunoregulatory receptor (VSIR, B7H5, VISTA; NCBI Gene ID: 64115); immunoglobulin superfamily member 11 (IGSF11, VSIG3; NCBI Gene ID: 152404); natural killer cell cytotoxicity receptor 3 ligand 1 (NCR3LG1, B7H6; NCBI Gene ID: 374383); HERV-H LTR-associating 2 (HHLA2, B7H7; NCBI Gene ID: 11148); inducible T cell co-stimulator (ICOS, CD278; NCBI Gene ID: 29851); inducible T cell co-stimulator ligand (ICOSLG, B7H2; NCBI Gene ID: 23308); TNF receptor superfamily member 4 (TNFRSF4, OX40; NCBI Gene ID: 7293); TNF superfamily member 4 (TNFSF4, OX40L; NCBI Gene ID: 7292); TNFRSF8 (CD30; NCBI Gene ID: 943), TNFSF8 (CD30L; NCBI Gene ID: 944); TNFRSF10A (CD261, DR4, TRAILR1; NCBI Gene ID: 8797), TNFRSF9 (CD137; NCBI Gene ID: 3604), TNFSF9 (CD137L; NCBI Gene ID: 8744); TNFRSF10B (CD262, DR5, TRAILR2; NCBI Gene ID: 8795), TNFRSF10 (TRAIL; NCBI Gene ID: 8743); TNFRSF14 (HVEM, CD270; NCBI Gene ID: 8764), TNFSF14 (HVEML; NCBI Gene ID: 8740); CD272 (B and T lymphocyte associated (BTLA); NCBI Gene ID: 151888); TNFRSF17 (BCMA, CD269; NCBI Gene ID: 608), TNFSF13B (BAFF; NCBI Gene ID: 10673); TNFRSF18 (GITR; NCBI Gene ID: 8784), TNFSF18 (GITRL; NCBI Gene ID: 8995); MHC class I polypeptide-related sequence A (MICA; NCBI Gene ID: 100507436); MHC class I polypeptide-related sequence B (MICB; NCBI Gene ID: 4277); CD274 (CD274, PDL1, PD-L1; NCBI Gene ID: 29126); programmed cell death 1 (PDCD1, PD1, PD-1; CD279; NCBI Gene ID: 5133); cytotoxic T-lymphocyte associated protein 4 (CTLA4, CD152; NCBI Gene ID: 1493); CD80 (B7-1; NCBI Gene ID: 941), CD28 (NCBI Gene ID: 940); nectin cell adhesion molecule 2 (NECTIN2, CD112; NCBI Gene ID: 5819); CD226 (DNAM-1; NCBI Gene ID: 10666); Poliovirus receptor (PVR) cell adhesion molecule (PVR, CD155; NCBI Gene ID: 5817); PVR related immunoglobulin domain containing (PVRIG, CD112R; NCBI Gene ID: 79037); T cell immunoreceptor with Ig and ITIM domains (TIGIT; NCBI Gene ID: 201633); T cell immunoglobulin and mucin domain containing 4 (TIMD4; TIM4; NCBI Gene ID: 91937); hepatitis A virus cellular receptor 2 (HAVCR2, TIMD3, TIM3; NCBI Gene ID: 84868); galectin 9 (LGALS9; NCBI Gene ID: 3965); lymphocyte activating 3 (LAG3, CD223; NCBI Gene ID: 3902); signaling lymphocytic activation molecule family member 1 (SLAMF1, SLAM, CD150; NCBI Gene ID: 6504); lymphocyte antigen 9 (LY9, CD229, SLAMF3; NCBI Gene ID: 4063); SLAM family member 6 (SLAMF6, CD352; NCBI Gene ID: 114836); SLAM family member 7 (SLAMF7, CD319; NCBI Gene ID: 57823); UL16 binding protein 1 (ULBP1; NCBI Gene ID: 80329); UL16 binding protein 2 (ULBP2; NCBI Gene ID: 80328); UL16 binding protein 3 (ULBP3; NCBI Gene ID: 79465); retinoic acid early transcript 1E (RAET1E; ULBP4; NCBI Gene ID: 135250); retinoic acid early transcript 1G (RAETIG; ULBP5; NCBI Gene ID: 353091); retinoic acid early transcript 1L (RAETIL; ULBP6; NCBI Gene ID: 154064); killer cell lectin like receptor C1 (KLRC1, NKG2A, CD159A; NCBI Gene ID: 3821); killer cell lectin like receptor K1 (KLRK1, NKG2D, CD314; NCBI Gene ID: 22914); killer cell lectin like receptor C2 (KLRC2, CD159c, NKG2C; NCBI Gene ID: 3822); killer cell lectin like receptor C3 (KLRC3, NKG2E; NCBI Gene ID: 3823); killer cell lectin like receptor C4 (KLRC4, NKG2F; NCBI Gene ID: 8302); killer cell immunoglobulin like receptor, two Ig domains and long cytoplasmic tail 1 (KIR2DL1; NCBI Gene ID: 3802); killer cell immunoglobulin like receptor, two Ig domains and long cytoplasmic tail 2 (KIR2DL2; NCBI Gene ID: 3803); killer cell immunoglobulin like receptor, two Ig domains and long cytoplasmic tail 3 (KIR2DL3; NCBI Gene ID: 3804); killer cell immunoglobulin like receptor, three Ig domains and long cytoplasmic tail 1 (KIR3DL1, KIR, CD158E1; NCBI Gene ID: 3811) (e.g., Lirilumab (IPH2102/BMS-986015), IPH-4102); and killer cell lectin like receptor D1 (KLRD1; NCBI Gene ID: 3824).

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with one or more blockers, antagonists or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). Lirilumab is an illustrative antibody that binds to and blocks KIR2DL1/2L3 receptors. In various embodiments, the fusion polypeptides, polynucleotides, vectors, LNPs, immunogenic compositions and/or pharmaceutical compositions, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with one or more blockers, antagonists or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A), e.g., monalizumab (IPH2201); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18 (11): 671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with anti-TIGIT antibodies, such as etigilimab, BMS-986207, tiragolumab (a.k.a., MTIG-7192A; RG-6058; RO 7092284), AGEN1307, AGEN1327, AGEN1777, COM-902, IBI-939, AB154, MG1131 and EOS884448 (EOS-448).

### TNF Receptor Superfamily (TNFRSF) Member Agonists or Activators

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with one or more agonists of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Example anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Example anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Example anti-TNFRSF9 (4-1BB, CD137) antibodies that can be co-administered include without limitation urelumab, utomilumab (PF-05082566), AGEN2373 and ADG-106.

Example anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi-and Tri-Specific Natural Killer (NK)-Cell Engagers

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BBAs appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices, et al., Methods Mol Biol. (2016) 1441:333-346; Fang, et al., Semin Immunol. (2017) 31:37-54. Examples of a trispecific NK cell engager (TRIKE) include OXS-3550, and CD16-IL-15-B7H3 TriKe.

Indoleamine-pyrrole-2,3-dioxygenase (IDO1) Inhibitors

In various embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, and shIDO-ST, EOS-200271, KHK-2455, LY-3381916.

### Toll-Like Receptor (TLR) Agonists

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Illustrative dual TLR7/TLR8 agonists that can be co-administered include CV8102, NKTR-262, telratolimod and BDB-001. Example TLR8 agonists that can be co-administered include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include without limitation AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. Examples of TLR4 agonist include G-100, and GSK-1795091. In some embodiments, the TLR agonist is a non-coding immunostimulatory polynucleotide selected from a pathogen-activated molecular pattern (PAMP), a cytosine-phosphate-guanosine (CpG) oligodeoxynucleotide, and an immunostimulatory RNA (isRNA, e.g., CV8102).

### STING Agonists, RIG-I and NOD2 Modulators

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a stimulator of interferon genes (STING) receptor (a.k.a, stimulator of interferon response cGAMP interactor 1 (STING1); transmembrane protein 173 (TMEM173); NCBI Gene ID: 340061) agonist. In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP) and cyclic-di-AMP.

In some embodiments, the additional therapeutic agent is an agonist of DExD/H-box helicase 58 (DDX58; a.k.a., RIG-I, RIG1, RIGI, RLR-1, SGMRT2; NCBI Gene ID: 23586). Illustrative RIG-I agonists include inarigivir soproxil (SB-9200; GS-9992); SB-40, SB-44, CV8102, ORI-7246, ORI-9350, ORI-7537, ORI-9020, ORI-9198, ORI-7170, RGT-100 and KIN1148, described by Hemann, et al., J Immunol May 1, 2016, 196 (1 Supplement) 76.1. Additional RIG-I agonists are described, e.g., in Elion, et al., Cancer Res. (2018) 78 (21): 6183-6195; and Liu, et al., J Virol. (2016) 90 (20): 9406-19. RIG-I agonists are commercially available, e.g., from Invivogen (invivogen.com). In some embodiments, the agents described herein are combined with a nucleotide binding oligomerization domain containing 2 (NOD2; NCBI Gene ID: 64127) agonist, such as inarigivir soproxil (SB-9200; GS-9992) and IR-103.

### LAG-3 and TIM-3 Inhibitors

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with an anti-TIM-3 (hepatitis A virus cellular receptor 2; HAVCR2; CD366, HAVcr-2, KIM-3, SPTCL, TIM3, TIMD-3, TIMD3, Tim-3; NCBI Gene ID: 84868) antibody, such as TSR-022, LY-3321367, MBG-453, INCAGN-2390.

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with anti-LAG-3 (lymphocyte-activating 3; LAG3; CD223; NCBI Gene ID: 3902) antibody, such as relatlimab (ONO-4482), LAG-525, MK-4280, REGN-3767, INCAGN2385.

### Interleukin or Cytokine Receptor Agonists

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a cytokine (e.g., interleukin) receptor agonist, such as IL-2, IL-7, IL-15, IL-10, IL-12, IL-18, IL-21, IFN-α, IFN-γ, GM-CSF, fms related receptor tyrosine kinase 3 (FLT3) receptor agonists, and combinations thereof. Examples of IL-2 receptor agonists that can be co-administered include proleukin (aldesleukin, IL-2); pegylated IL-2 (e.g., NKTR-214); modified variants of IL-2 (e.g., THOR-707), bempegaldesleukin, AIC-284, ALKS-4230, CUI-101, Neo-2/15. Examples of IL-15 receptor agonists that can be co-administered include ALT-803 (nogapendekin alfa), NKTR-255, and hetIL-15, interleukin-15/Fc fusion protein, AM-0015, NIZ-985, SO-C101, IL-15 Synthorin (pegylated Il-15), P-22339, and an IL-15-PD-1 fusion protein N-809. Examples of IL-7 receptor agonist that can be co-administered include CYT-107.

Examples of additional immune-based therapies that can be combined with an agent of this disclosure include interferon alfa; interferon alfa-2b; interferon alfa-n3; pegylated interferon alfa; interferon gamma; fms related tyrosine kinase 3 (FLT3) agonists (e.g., GS-3583, CDX-301); gepon; normferon, peginterferon alfa-2a, peginterferon alfa-2b, RPI-MN.

### Phosphatidylinositol 3-Kinase (PI3K) Inhibitors

In some embodiments, the immunogenic polypeptides, polynucleotides encoding such polypeptides, vectors, LNPs, PNPs, nanoemulsions, and immunogenic compositions comprising such polypeptides or polynucleotides, as described herein, are combined or co-administered with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p1 10-alpha; NCBI Gene ID: 5290); phosphatidylinositol-4,5-bisphosphate 3-kinase catalytic subunit beta (PIK3CB, P110BETA, PI3K, PI3KBETA, PIK3C1; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PI3K; Gene ID: 5494); and/or phosphatidylinositol-4,5-bisphosphate 3-kinase catalytic subunit delta (PIK3CD, APDS, IMD14, P110DELTA, PI3K, p110D, NCBI Gene ID: 5293). In some embodiments, the PI3K inhibitor is a pan-PI3K inhibitor. Examples of PI3K inhibitors include without limitation, ACP-319, AEZA-129, AMG-319, AS252424, AZD8186, BAY 1082439, BEZ235, bimiralisib (PQR309), buparlisib (BKM120), BYL719 (alpelisib), carboxyamidotriazole orotate (CTO), CH5132799, CLR-457, CLR-1401, copanlisib (BAY 80-6946), DS-7423, duvelisib (IPI-145), fimepinostat (CUDC-907), gedatolisib (PF-05212384), GDC-0032, GDC-0084 (RG7666), GDC-0077, pictilisib (GDC-0941), GDC-0980, GSK2636771, GSK2269577, idelalisib (Zydelig®), INCB040093, INCB50465, IPI-443, IPI-549, KAR4141, LY294002, LY3023414, NERLYNX® (neratinib), nemiralisib (GSK2269557), omipalisib (GSK2126458, GSK458), OXY111A, panulisib (P7170, AK151761), PA799, perifosine (KRX-0401), Pilaralisib (SAR245408; XL147), puquitinib mesylate (XC-302), SAR260301, seletalisib (UCB-5857), serabelisib (INK-1117,MLN-1117,TAK-117), SF1126, sonolisib (PX-866), RG7604, rigosertib sodium (ON-01910 sodium), RP5090, tenalisib (RP6530), RV-1729, SRX3177, taselisib, TG100115, umbralisib (TGR-1202), TGX221, voxtalisib (SAR245409), VS-5584, WX-037, X-339, X-414, XL499, XL756, wortmannin, ZSTK474, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).

alpha-4/beta-7 antagonists

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with an alpha-4/beta-7 antagonist. Examples of Integrin alpha-4/beta-7 antagonists that can be combined with an agent of this disclosure include PTG-100, TRK-170, abrilumab, etrolizumab, carotegrast methyl, and vedolizumab.

### Inhibitor of CD47

In various embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with an inhibitor of CD47 (IAP, MER6, OA3; NCBI Gene ID: 961; UniProt Q08722) or an agent that disrupts the binding of CD47 to SIRPa. Examples of CD47 inhibitors include without limitation to anti-CD47 mAbs (Vx-1004), anti-human CD47 mAbs (CNTO-7108), CC-90002, CC-90002-ST-001, humanized anti-CD47 antibody (Hu5F9-G4; magrolimab), NI-1701, NI-1801, RCT-1938, ALX-148, TTI-621, RRx-001, DSP-107, VT-1021, TTI-621, TTI-622, IMM-02 and SGN-CD47M, as well as CD47 targeting agents described in Intl. Patent Publ. Nos. WO199727873, WO199940940, WO2002092784, WO2005044857, WO2009046541, WO2010070047, WO2011143624, WO2012170250, WO2013109752, WO2013119714, WO2014087248, WO2015191861, WO2016022971, WO2016023040, WO2016024021, WO2016081423, WO2016109415, WO2016141328, WO2016188449, WO2017027422, WO2017049251, WO2017053423, WO2017121771, WO2017194634, WO2017196793, WO2017215585, WO2018075857, WO2018075960, WO2018089508, WO2018095428, WO2018137705, WO2018233575, WO2019027903, WO2019034895, WO2019042119, WO2019042285, WO2019042470, WO2019086573, WO2019108733, WO2019138367, WO2019144895, WO2019157843, WO2019179366, WO2019184912, WO2019185717, WO2019201236, WO2019238012, WO2019241732, WO2020019135, WO2020036977, WO2020043188 and WO2020009725.

Examples bi-specific antibodies targeting CD47 that can be combined or co-administered include without limitation IBI-322 (CD47/PD-L1), IMM-0306 (CD47/CD20), TJ-LIC4 (CD47/PD-L1), HX-009 (CD47/PD-1), PMC-122 (CD47/PD-L1), PT-217, (CD47/DLL3), IMM-26011 (CD47/FLT3), IMM-0207 (CD47/VEGF), IMM-2902 (CD47/HER2), BH29xx (CD47/PD-L1), IMM-03 (CD47/CD20), IMM-2502 (CD47/PD-L1), HMBD-004B (CD47/BCMA), HMBD-004A (CD47/CD33). Examples of anti-CD47antibodies, such as IBI-188, TJC-4, SHR-1603, HLX-24, LQ-001, IMC-002, ZL-1201, IMM-01, B6H12, GenSci-059, TAY-018, PT-240, 1F8-GMCSF, SY-102, KD-015.

### HIV Targeting Antibodies

In various embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with HIV antibodies, bispecific antibodies, and/or “antibody-like” therapeutic proteins. Examples of HIV antibodies, bispecific antibodies, and “antibody-like” therapeutic proteins that can be combined with an agent of this disclosure include DARTs®, DUOBODIES®, BITES®, XmAbs®, TandAbs®, Fab derivatives, bNAbs (broadly neutralizing HIV-1 antibodies), TMB-360, and those targeting HIV gp120 or gp41, antibody-Recruiting Molecules targeting HIV, anti-CD63 monoclonal antibodies, anti-GB virus C antibodies, anti-GP120/CD4, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), ibalizumab, Immuglo, MB-66.

In certain embodiments, the co-administered antibody or antigen-binding fragment thereof, or an antigen-binding molecule, is or is derived from human neutralizing antibodies (e.g., monoclonal) that target HIV-1. A “neutralizing antibody” is one that can neutralize the ability of HIV to initiate and/or perpetuate an infection in a host and/or in target cells in vitro. The disclosure provides neutralizing monoclonal human antibodies, wherein the antibody recognizes an antigen from HIV, e.g., a gp120 polypeptide. In certain embodiments, a “neutralizing antibody” may inhibit the entry of HIV-1 virus, e.g., SF162 and/or JR-CSF, with a neutralization index>1.5 or >2.0 (Kostrikis L G et al., J. Virol.,70 (1): 445-458 (1996)).

In some embodiments, the co-administered antibody or antigen-binding fragment thereof, or an antigen-binding molecule, is or is derived from human broadly neutralizing antibodies (e.g., monoclonal) that target HIV-1. By “broadly neutralizing antibodies” are meant antibodies that neutralize more than one HIV-1 virus species (from diverse clades and different strains within a clade) in a neutralization assay. A broad neutralizing antibody may neutralize at least 2, 3, 4, 5, 6, 7, 8, 9 or more different strains of HIV-1, the strains belonging to the same or different clades. Illustrative broadly neutralizing antibodies (bNAbs) which can be co-administered as an additional therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Illustrative bNAbs that can be co-administered include without limitation 12A12, 12A21, NIH45-46, bANC131, 8ANC134, IB2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Sajadi, et al., Cell. (2018) 173 (7): 1783-1795; Sajadi, et al., J Infect Dis. (2016) 213 (1): 156-64; Klein et al., Nature, 492 (7427): 118-22 (2012), Horwitz et al., Proc Natl Acad Sci USA, 110 (41): 16538-43 (2013), Scheid, et al., Science, 333:1633-1637 (2011), Scheid, et al., Nature, 458:636-640 (2009), Eroshkin et al, Nucleic Acids Res., 42 (Database issue): DI 133-9 (2014), Mascola et al., Immunol Rev., 254 (1): 225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site), which are hereby incorporated herein by reference in their entireties for all purposes.

In some embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a broadly neutralizing antibody (bNAb)) that binds to an epitope or region of gp120 selected from the group consisting of: (i) the third variable loop (V3) and/or high mannose patch comprising a N332 oligomannose glycan; (ii) second variable loop (V2) and/or Env trimer apex; (iii) CD4 binding site (CD4bs); (iv) gp120/gp41 interface; or (v) silent face of gp120. The foregoing epitopes or regions of gp120 bound by broadly neutralizing antibodies are described, e.g., in McCoy, Retrovirology (2018) 15:70; Sok and Burton, Nat Immunol. 2018 19 (11): 1179-1188; Possas, et al., Expert Opin Ther Pat. 2018 July; 28 (7): 551-560; and Stephenson and Barouch, Curr HIV/AIDS Rep (2016) 13:31-37, which are hereby incorporated herein by reference in their entirety for all purposes.

In some embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a broadly neutralizing antibody (bNAb) that binds to an epitope or region of gp120 in the third variable loop (V3) and/or high mannose patch comprising a N332 oligomannose glycan and competes with or comprises VH and VL regions from an antibody selected from the group consisting of GS-9722, PGT-121.60, PGT-121.66, PGT-121, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. Additional broadly neutralizing antibodies that bind to gp120 in the third variable loop (V3) and/or high mannose patch comprising a N332 oligomannose glycan and which can be used as the second antibody or antigen-binding fragment thereof are described, e.g., in WO 2012/030904; WO 2014/063059; WO 2016/149698; WO 2017/106346; WO 2018/075564, WO 2018/125813 and WO 2018/237148, which are hereby incorporated herein by reference in their entireties for all purposes.

In some embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a broadly neutralizing antibody (bNAb)) that binds to an epitope or region of gp120 in the CD4 binding site (CD4bs) and competes with or comprises CDRs and/or VH and VL regions from an antibody selected from the group consisting of b12, F105, VRC01, VRC07, VRC07-523, VRC03, VRC06, VRC06b01 VRC08, VRC0801, NIH45-46, GS-9723, 3BNC117, 3BNC60, VRC-PG04, PGV04; CH103, 44-VRC13.01, 1NC9, 12A12, N6, N49-P7, NC-Cow1, IOMA, CH235 and CH235.12, N49P6, N49P7, N49P11, N49P9 and N60P25.

In some embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a broadly neutralizing antibody (bNAb)) that binds to an epitope or region of gp120 in the second variable loop (V2) and/or Env trimer apex and competes with or comprises VH and VL regions from an antibody selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01. Additional broadly neutralizing antibodies that bind to gp120 in the second variable loop (V2) and/or Env trimer apex and which can be used as the second antibody or antigen-binding fragment thereof are described, e.g., in WO 2010/107939; WO 2012/030904; WO 2018/075564 and WO 2018/125813, which are hereby incorporated herein by reference in their entireties for all purposes.

In some embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a broadly neutralizing antibody (bNAb)) that binds to an epitope or region of gp120 in the gp120/gp41 interface and competes with or comprises VH and VL regions from an antibody selected from the group consisting of PGT-151, CAP248-2B, 35022, 8ANC195, ACS202, VRC34 and VRC34.01. Additional broadly neutralizing antibodies that bind to gp120 in the gp120/gp41 interface and which can be used as the second antibody or antigen-binding fragment thereof are described, e.g., in WO 2011/038290; WO 2012/030904 and WO2017/079479, which are hereby incorporated herein by reference in their entireties for all purposes.

In some embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, combined or co-administered with a broadly neutralizing antibody (bNAb)) that binds to an epitope or region of the gp120 silent face and competes with or comprises VH and VL regions from an antibody selected from the group consisting of VRC-PG05 and SF12. See, e.g., Schoofs, et al., “Broad and Potent Neutralizing Antibodies Recognize the Silent Face of the HIV Envelope,” Immunity (2019) May 14. pii: S1074-7613 (19) 30194-3 (PMID 31126879).

In some embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a broadly neutralizing antibody (bNAb)) that binds to an epitope or region of gp41 in the membrane proximal region (MPER). Additional broadly neutralizing antibodies that bind to gp41 in the MPER and which can be used as the second antibody or antigen-binding fragment thereof are described, e.g., in WO 2011/034582; WO 2011/038290; WO 2011/046623 and WO 2013/070776, which are hereby incorporated herein by reference in their entireties for all purposes.

In some embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a broadly neutralizing antibody (bNAb)) that binds to an epitope or region of gp41 in the membrane proximal region (MPER) and competes with or comprises VH and VL regions from an antibody selected from the group consisting of 10E8, 10E8v4, 10E8-5R-100cF, 4E10, DH511.11P, 2F5, 7b2, and LN01.

In some embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined or co-administered with a broadly neutralizing antibody (bNAb)) that binds to an epitope or region of the gp41 fusion peptide and competes with or comprises VH and VL regions from an antibody selected from the group consisting of VRC34 and ACS202.

Examples of additional antibodies that can be co-administered include bavituximab, UB-421, BF520.1, CH01, CH59, C2F5, C4E10, C2F5+C2G12+C4E10, 3BNC117, 3BNC117-LS, 3BNC60, DH270.1, DH270.6, DID2, 10-1074-LS, GS-9722, DH411-2, BG18, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, VRC-eCD4-Ig, IOMA, CAP256-VRC26.25, DRVIA7, VRC-HIVMAB080-00-AB, HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Example of HIV bispecific and trispecific antibodies include MGD014, B12BiTe, TMB-bispecific, SAR-441236, VRC-01/PGDM-1400/10E8v4, 10E8.4/iMab, 10E8v4/PGT121-VRC01.

In some embodiments, the bNAbs can be expressed in vivo in the patient. Examples of in vivo delivered bNAbs include AAV8-VRC07; mRNA encoding anti-HIV antibody VRC01; and engineered B-cells encoding 3BNC117 (Hartweger et al, J. Exp. Med. 2019, 1301).

### Pharmacokinetic Enhancers

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with a pharmacokinetic enhancer. Examples of pharmacokinetic enhancers that can be combined with an agent of this disclosure include cobicistat and ritonavir.

### Additional Therapeutic Agents

Examples of additional therapeutic agents that can be combined with the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, include the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US20140221356 (Gilead Sciences), US20100143301 (Gilead Sciences) and WO 2013/091096 (Boehringer Ingelheim).

### HIV Vaccines

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with an HIV vaccine. Examples of HIV vaccines that can be combined with an agent of this disclosure include peptide vaccines, recombinant subunit protein vaccines, live vector vaccines, DNA vaccines, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of HIV vaccines that can be co-administered include: rgp120 (AIDSVAX), ALVAC HIV (vCP1521)/AIDSVAX B/E (gp120) (RV144), monomeric gp120 HIV-1 subtype C vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), gp140 [delta] V2.TV1+MF-59, rVSVIN HIV-1 gag vaccine, SeV-Gag vaccine, AT-20, DNK-4, ad35-Grin/ENV, TBC-M4, HIVAX, HIVAX-2, NYVAC—HIV-PT1, NYVAC—HIV-PT4, DNA-HIV-PT123, rAAV1-PG9DP, GOVX-B11, GOVX-B21, TVI-HIV-1, Ad-4 (Ad4-env Clade C+Ad4-mGag), Paxvax, EN41-UGR7C, EN41-FPA2, PreVaxTat, AE-H, MYM-V101, CombiHIVvac, ADVAX, MYM-V201, MVA-CMDR, DNA-Ad5 gag/pol/nef/nev (HVTN505), MVATG-17401, ETV-01, CDX-1401, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, arenavirus vector-based vaccines (e.g., described in WO 2009/083210; WO 2015/183895; WO 2016/075250; WO 2017/198726; and U.S. Pat. No. 9,943,585), MVA-BN HIV-1 vaccine regimen, UBI HIV gp120, mRNA based prophylactic vaccines, and TBL-1203HI.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In one embodiment, an agent disclosed herein, or a pharmaceutically acceptable salt thereof, is combined with one, two, three, four or more additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY (bictegravir+emtricitabine+tenofovir alafenamide), adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In a certain embodiment, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with abacavir sulfate, tenofovir, tenofovir disoproxil, tenofovir disoproxil fumarate, tenofovir disoproxil hemifumarate, tenofovir alafenamide, or tenofovir alafenamide hemifumarate.

In another embodiment, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with tenofovir, tenofovir disoproxil, tenofovir disoproxil fumarate, tenofovir alafenamide, or tenofovir alafenamide hemifumarate.

In yet another embodiment, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with a first additional therapeutic agent selected from the group consisting of abacavir sulfate, tenofovir, tenofovir disoproxil, tenofovir disoproxil fumarate, tenofovir alafenamide, and tenofovir alafenamide hemifumarate, and a second additional therapeutic agent selected from the group consisting of emtricitabine and lamivudine.

In another embodiment, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with a first additional therapeutic agent selected from the group consisting of tenofovir, tenofovir disoproxil, tenofovir disoproxil fumarate, tenofovir alafenamide, and tenofovir alafenamide hemifumarate, and a second additional therapeutic agent, wherein the second additional therapeutic agent is emtricitabine.

In some embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of dendritic cell therapy include AGS-004. CCR5 gene editing agents include SB-728T. CCR5 gene inhibitors include Cal-1. In some embodiments, C34-CCR5/C34-CXCR4 expressing CD4-positive T-cells are co-administered with the one or more fusion polypeptides. In some embodiments, the agents described herein are co-administered with AGT-103-transduced autologous T-cell therapy or AAV-eCD4-Ig gene therapy.

### Gene Editors

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T-cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include VC-CAR-T, CMV-N6-CART, anti-CD4 CART-cell therapy, CD4 CAR+C34-CXCR4+CCR5 ZFN T-cells, autologous hematopoietic stem cells genetically engineered to express a CD4 CAR and the C46 peptide.

### TCR-T-cell Therapy

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with a population of TCR-T-cells. TCR-T-cells are engineered to target HIV derived peptides present on the surface of virus-infected cells, for example ImmTAV.

### B-cell Therapy

In certain embodiments, the one or more fusion polypeptides, or polynucleotides encoding such fusion polypeptides, or an expression vector, viral expression vector, viral vector, SAM, LNP, PNP or nanoemulsion comprising such polynucleotides, as disclosed herein, are combined with a population of B cells genetically modified to express broadly neutralizing antibodies, such as 3BNC117 (Hartweger et al, J. Exp. Med. 2019, 1301, Moffett et al., Sci. Immunol. 4, eaax0644 (2019) 17 May 2019).

### Kits

Further provided are kits comprising one or more unitary doses of one or more of the fusion polypeptides, as described herein, or polynucleotides encoding such fusion polypeptides, as described herein, or an expression vector, viral expression vector, viral vector, SAM, LNP, PNP or nanoemulsion comprising such polynucleotides, as described herein. In some embodiments, the kit comprises two or more unitary doses of one or more of the fusion polypeptides, as described herein, or two or more polynucleotides encoding such fusion polypeptides, as described herein, or two or more expression vectors, viral expression vectors, viral vectors, SAMs, LNPs, PNPs or nanoemulsions comprising such polynucleotides, as described herein. In some embodiments, the one or more unitary doses are in a single container. In some embodiments, the one or more unitary doses are in two or more separate containers. In certain embodiments, the unitary doses can be the same or different, e.g., can comprise the same or different unitary doses, e.g., can comprise polypeptides, polynucleotides, vectors or combinations thereof.

In various embodiments, the kit comprises one or more pharmaceutical packs or one or more containers (e.g., vials, ampules, pre-loaded syringes) containing one or more of the ingredients of the pharmaceutical compositions described herein, such as one or more of the fusion polypeptides, as described herein, or one or more polynucleotides encoding such fusion polypeptides, as described herein, or one or more expression vectors, viral expression vectors, viral vectors, SAMs, LNPs, PNPs or nanoemulsions comprising such polynucleotides, as described herein. In various embodiments, the kit comprises one or more containers comprising the one or more of the fusion polypeptides, as described herein, or one or more polynucleotides encoding such fusion polypeptides, as described herein, or one or more expression vectors, viral expression vectors, viral vectors, SAMs, LNPs, PNPs or nanoemulsions comprising such polynucleotides, in an aqueous solution. In various embodiments, the kit comprises one or more containers comprising the one or more of the fusion polypeptides, as described herein, or one or more polynucleotides encoding such fusion polypeptides, as described herein, or one or more expression vectors, viral expression vectors, viral vectors, SAMs, LNPs, PNPs or nanoemulsions comprising such polynucleotides, as described herein, in lyophilized form.

In some embodiments, the kit comprises one or more unitary doses of one or more viral vectors capable of expressing the fusion polypeptides. In some embodiments, the unitary doses of the one or more viral vectors are in the range of about 10³ to about 10¹² viral focus forming units (FFU) or plaque forming units (PFU) or infectious units (IU) or viral particles (vp), e.g. from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration.

In some embodiments, the kit comprises two or more polynucleotides encoding or two or more viral vectors or SAMs expressing the fusion polypeptides, the fusion polypeptides comprising: (1) one or more fusion polypeptides comprising or consisting of the following polypeptide segments in sequential order, from N-terminus to C-terminus, optionally joined or connected by one or more linkers: SEQ ID NOs: 70, 76, 94, 151 and 161; or SEQ ID NOs: 71, 77, 95, 152 and 162; and (2) one or more fusion polypeptides comprising or consisting of the following polypeptide segments in sequential order, from N-terminus to C-terminus, optionally joined or connected by one or more linkers: SEQ ID NOs: 188, 305, 28, 41, 294, 4, 176, 11, 319, 259, 282, 223, 213 and 37; SEQ ID NOs: 188, 305, 28, 41 and 294; SEQ ID NOs: 4, 176, 11, 319, 259, 282, 223, 213 and 37; SEQ ID NOs: 189, 306, 29, 42, 295, 5, 177, 12, 320, 260, 283, 224, 214 and 38; SEQ ID NOs: 189, 306, 29, 42 and 295; SEQ ID NOs: 5, 177, 12, 320, 260, 283, 224, 214 and 38; SEQ ID NOs: 305, 319, 259, 282, 223, 213, 294, 176 and 188; SEQ ID NOs: 306, 320, 260, 283, 224, 214, 295, 177 and 189; SEQ ID NOs: 305, 294, 223, 213, 176, 259, 319, 188 and 282; SEQ ID NOs: 306, 295, 224, 214, 177, 260, 320, 189 and 283; SEQ ID NOs: 305, 294, 319, 259, 282, 223, 176, and 188; SEQ ID NOs: 306, 295, 320, 260, 283, 224, 177 and 189; SEQ ID NOs: 305, 223, 294, 176, 259, 319, 188 and 282; or SEQ ID NOs: 306, 224, 295, 177, 260, 320, 189 and 283.

In some embodiments, the kit comprises two or more polynucleotides encoding or two or more viral vectors or SAMs expressing the fusion polypeptides, the fusion polypeptides comprising: (1) One or more fusion polypeptides comprising an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430; and (2) one or more fusion polypeptides comprising an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410.

In some embodiments, the kit comprises one or more viral vectors or SAMs, wherein each viral vector or SAM comprises two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

In some embodiments, the kit comprises one or more viral vectors or SAMs, wherein each viral vector or SAM comprises two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the amino acid sequence of SEQ ID NOs: 527-528.

In some embodiments, the kit comprises one or more viral vectors or SAMs, wherein each viral vector comprises a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NOs: 520-521.

In some embodiments, the kit comprises one or more viral vectors or SAMs, wherein each viral vector or SAM a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NOs: 522-523.

In some embodiments, the kit comprises one or more viral vectors or SAMs, wherein each viral vector or SAM comprises a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the nucleic acid sequence of SEQ ID NOs: 524-526.

In some embodiments, the kit comprises one or more polynucleotides encoding the fusion polypeptides, or one or more expression vectors, viral expression vectors, viral vectors, SAMs, LNPs, PNPs, or nanoemulsions comprising such polynucleotides, the fusion polypeptides comprising or consisting of the following polypeptide segments in sequential order, from N-terminus to C-terminus, optionally joined or connected by one or more linkers: SEQ ID NOs: 201, 78, 107, 96, 229, 172, 327, 6, 333, 243, 331, 192, 265, 311, 137, 15, 123, 30, 336, 302, 153, 219, 298, 121, 230, 240, 60, 241, 276, 113, 99, 21, 217 and 215; SEQ ID NOs: 78, 296, 1, 339, 197, 329, 232, 323, 303, 234, 90, 261, 274, 238, 211, 325, 137, 227, 209, 190, 341, 57, 225, 27, 210, 119, 19, 165, 334, 117, 153, 10, 97 and 300; or SEQ ID NOs: 296, 1, 78, 197, 339, 227, 261, 274, 238, 325, 137, 329, 303, 234, 90, 232, 27, 57, 225, 323, 190, 341, 119, 19, 165, 334, 117, 153, 10, 97 and 300.

In some embodiments, the kit comprises one or more polynucleotides encoding the fusion polypeptides, or one or more expression vectors, viral expression vectors, viral vectors, SAMs, LNPs, PNPs, or nanoemulsions comprising such polynucleotides, the fusion polypeptides comprising or consisting of an amino acid sequence of any one of SEQ ID NOs: 367-377, 411, 422-424 and 431-435, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 367-377, 411, 422-424 and 431-435.

In some embodiments, the kit comprises one or more polynucleotides encoding the fusion polypeptides, or one or more expression vectors, viral expression vectors, viral vectors, SAMs, LNPs, PNPs, or nanoemulsions comprising such polynucleotides, the fusion polypeptides comprising or consisting of an amino acid sequence of any one of SEQ ID NOs: 367-377, 411, 422-424 and 431-435, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 367-377, 411, 422-424 and 431-435.

In some embodiments, the kit comprises one or more polynucleotides encoding the fusion polypeptides, or one or more expression vectors, viral expression vectors, viral vectors, SAMs, LNPs, PNPs, or nanoemulsions comprising such polynucleotides, the fusion polypeptides comprising or consisting of an amino acid sequence of any one of SEQ ID NOs: 527-528, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs. 527-528.

In some embodiments, the kit comprises one or more polynucleotides, or one or more expression vectors, viral expression vectors, viral vectors, SAMs, LNPs, PNPs, or nanoemulsions comprising such polynucleotides, the polynucleotides comprising or consisting of a nucleic acid sequence of any one of SEQ ID NOs: 520-521, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 520-521.

In some embodiments, the kit comprises one or more polynucleotides, or one or more expression vectors, viral expression vectors, viral vectors, SAMs, LNPs, PNPs, or nanoemulsions comprising such polynucleotides, the polynucleotides comprising or consisting of a nucleic acid sequence of any one of SEQ ID NOs: 522-521, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 522-523.

In some embodiments, the kit comprises one or more polynucleotides, or one or more expression vectors, viral expression vectors, viral vectors, SAMs, LNPs, PNPs, or nanoemulsions comprising such polynucleotides, the polynucleotides comprising or consisting of a nucleic acid sequence of any one of SEQ ID NOs: 524-526, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 524-526.

In some embodiments, the kit further comprises one or more unitary doses of one or more additional therapeutic agents. For example, in some embodiments, the kit comprises one or more agonists or activators of one or more toll-like receptors (TLRs). In some embodiments, the TLR agonist or activator is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, the TLR agonist is a non-coding immunostimulatory polynucleotide selected from a pathogen-activated molecular pattern (PAMP), a cytosine-phosphate-guanosine (CpG) oligodeoxynucleotide, and an immunostimulatory RNA (isRNA, e.g., CV8102).

In some embodiments, the kit comprises one or more interleukin receptor agonists of an interleukin selected from IL-2, IL-7, IL-12, IL-15, IL-18, IL-21, IFN-α, IFN γ, colony stimulating factor 2 (CSF2; a.k.a., GM-CSF) and FLT3LG, e.g., one or more cytokines selected from the group consisting of IL-2, IL-7, IL-12, IL-15, IL-18, IL-21, IFN-α, IFN-γ, GM-CSF, FLT3LG, and combinations and functional variants thereof.

In some embodiments, the kit comprises one or more antagonists or inhibitors of an inhibitory immune checkpoint protein or receptor and/or one or more activators or agonists of a stimulatory immune checkpoint protein or receptor. In some embodiments, the one or more immune checkpoint proteins or receptors are selected from the group consisting of: CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); and SLAM family member 7 (SLAMF7). In some embodiments, the kit comprises one or more blockers, antagonists or inhibitors of one or more T-cell inhibitory immune checkpoint proteins or receptors. In some embodiments, the T-cell inhibitory immune checkpoint proteins or receptors are selected from the group consisting of CD274 (CD274, PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). Lirilumab is an illustrative antibody that binds to and blocks KIR2DL1/2L3 receptors. In some embodiments, the kit comprises one or more agonists or activators of one or more T-cell stimulatory immune checkpoint proteins or receptors. In some embodiments, the T-cell stimulatory immune checkpoint proteins or receptors are selected from the group consisting of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the kit comprises one or more blockers, antagonists or inhibitors of one or more NK-cell inhibitory immune checkpoint proteins or receptors. In some embodiments, the NK-cell inhibitory immune checkpoint proteins or receptors are selected from the group consisting of killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A), e.g., monalizumab (IPH2201); and killer cell lectin like receptor D1 (KLRD1, CD94). In some embodiments, the kit comprises one or more agonists or activators of one or more NK-cell stimulatory immune checkpoint proteins or receptors. In some embodiments, the NK-cell stimulatory immune checkpoint proteins or receptors are selected from CD16, CD226 (DNAM-1); killer cell lectin like receptor K1 (KLRK1, NKG2D, CD314); and SLAM family member 7 (SLAMF7). In some embodiments, the one or more immune checkpoint inhibitors comprises a proteinaceous inhibitor of PD-L1 (CD274), PD-1 (PDCD1) or CTLA4. In some embodiments, the proteinaceous inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4) and AK-104 (CTLA4/PD-1). In some embodiments, the proteinaceous inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFB-EC domain), CA-170 (PD-L1/VISTA), CDX-527 (CD27/PD-L1), LY-3415244 (TIM3/PDL1), and INBRX-105 (4-1BB/PDL1). In some embodiments, the one or more immune checkpoint inhibitors comprises a small molecule inhibitor of CD274 (PDL1, PD-L1), programmed cell death 1 (PDCD1, PD1, PD-1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

In some embodiments, the kit comprises one or more anti-viral agents. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, and capsid inhibitors.

Optionally associated with such kit(s) can be a notice in the form prescribed by a governmental agency regulating the manufacture, use or sale of pharmaceuticals or biological products, which notice reflects approval by the agency of manufacture, use or sale for human administration.

### EXEMPLARY EMBODIMENTS

Disclosed herein are polynucleotides comprising any one of the HIV immunogen nucleic acid sequences of SEQ ID NOs: 524-526. Disclosed herein are polynucleotides comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the HIV immunogen nucleic acid sequences of SEQ ID NOs: 524-526, wherein percent identity is across the full length of the HIV immunogen nucleic acid sequence of SEQ ID NOs: 524-526. Disclosed herein are polynucleotides comprising a codon-optimized nucleic acid sequence of any one of the HIV immunogen nucleic acid sequences of SEQ ID NOs: 524-526. In some embodiments, the polynucleotide comprises DNA. In some embodiments, the polynucleotide comprises cDNA. In some embodiments, the polynucleotide comprises mRNA.

Disclosed herein are polynucleotides comprising an HIV immunogen nucleic acid sequence that encodes any one of the HIV immunogen amino acid sequences of SEQ ID NOS: 527-528. Disclosed herein are polynucleotides comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 527-528, wherein percent identity is across the full length of the nucleic acid sequence of the polypeptide encoding any one of the HIV immunogen amino acid sequence of SEQ ID NOs: 527-528. Disclosed herein are polynucleotides comprising a codon-optimized nucleic acid sequence of an HIV immunogen nucleic acid sequence that encodes any one of the HIV immunogen amino acid sequences of SEQ ID NOs: 527-528. In some embodiments, the polynucleotide comprises DNA. In some embodiments, the polynucleotide comprises cDNA. In some embodiments, the polynucleotide comprises mRNA.

Further disclosed herein is self-amplifying RNA (SAM) comprising a polynucleotide, wherein the polynucleotide comprises any one of the nucleic acid sequences of SEQ ID NOs: 522-523. Further disclosed herein is a self-amplifying RNA (SAM) comprising a polynucleotide, wherein the polynucleotide comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523. Further disclosed herein is self-amplifying RNA (SAM) comprising a polynucleotide, wherein the polynucleotide comprises a codon-optimized nucleic acid sequence of any one of the nucleic acid sequences of SEQ ID NOs: 522-523.

Further disclosed herein is a self-amplifying mRNA (SAM) comprising a polynucleotide comprising any one of the HIV immunogen nucleic acid sequences of SEQ ID NOs: 524-526. Further disclosed herein is a self-amplifying mRNA (SAM) comprising a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the HIV immunogen nucleic acid sequences of SEQ ID NOs: 524-526, wherein percent identity is across the full length of the HIV immunogen nucleic acid sequence of SEQ ID NOs: 524-526. Further disclosed herein is a self-amplifying mRNA (SAM) comprising a polynucleotide comprising a codon-optimized nucleic acid sequence of any one of the HIV immunogen nucleic acid sequences of SEQ ID NOs: 524-526. In some embodiments, the SAM is derived from a virus. In some embodiments, the virus is replication deficient. In some embodiments, the virus is an RNA virus. In some embodiments, the RNA virus is positive-sense single-stranded RNA virus. In some embodiments, the virus is selected from an alphavirus, flavivirus, nidovirus, nodamura virus, and picornavirus. In some embodiments, the alphavirus is selected from an Old World (OW) alphavirus and New World (NW) alpha virus. In some embodiments, the OW alphavirus is selected from Chikunguyna virus (CHIKV), Ross River virus (RRV), Semliki Forest virus (SFV), and Sindbis virus (SINV). In some embodiments, the NW alphavirus is selected from Venezuelan equine encephalitis virus (VEEV), eastern equine encephalitis virus (EEEV), and western equine encephalitis virus (WEEV). In some embodiments, the SAM is derived from Venezuelan equine encephalitis virus (VEEV). In some embodiments, the virus comprises a modified viral genome. In some embodiments, the modified viral genome comprises a deletion of one or more genes encoding one or more viral structural proteins. In some embodiments, the modified viral genome is produced by replacing one or more viral structural proteins with any of the polynucleotides disclosed herein. In some embodiments, the modified viral genome is produced by replacing one or more viral structural proteins with a polynucleotide comprising the nucleic acid sequence of SEQ ID NOs: 524-526. In some embodiments, the modified viral genome is produced by replacing one or more viral structural proteins with a polynucleotide comprising a codon-optimized nucleic acid sequence of the nucleic acid sequence of SEQ ID NOs: 524-526. In some embodiments, the modified viral genome is produced by replacing one or more viral structural proteins with a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 524-526, wherein percent identity is across the full length of SEQ ID NOs: 524-526. In some embodiments, the SAM further comprises a promoter sequence. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529.

Further disclosed herein is a self-amplifying mRNA (SAM) comprising a polynucleotide comprising an HIV immunogen nucleic acid sequence that encodes any one of the HIV immunogen amino acid sequences of SEQ ID NOs: 527-528. Further disclosed herein is a self-amplifying mRNA (SAM) comprising a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 527-528, wherein percent identity is across the full length of the nucleic acid sequence of the polypeptide encoding any one of the HIV immunogen amino acid sequence of SEQ ID NOs: 527-528. Further disclosed herein is a self-amplifying mRNA (SAM) comprising a polynucleotide comprising a codon-optimized nucleic acid sequence of an HIV immunogen nucleic acid sequence that encodes any one of the HIV immunogen amino acid sequences of SEQ ID NOs: 527-528. In some embodiments, the SAM is derived from a virus. In some embodiments, the virus is replication deficient. In some embodiments, the virus is an RNA virus. In some embodiments, the RNA virus is positive-sense single-stranded RNA virus. In some embodiments, the virus is selected from an alphavirus, flavivirus, nidovirus, nodamura virus, and picornavirus. In some embodiments, the alphavirus is selected from an Old World (OW) alphavirus and New World (NW) alpha virus. In some embodiments, the OW alphavirus is selected from Chikunguyna virus (CHIKV), Ross River virus (RRV), Semliki Forest virus (SFV), and Sindbis virus (SINV). In some embodiments, the NW alphavirus is selected from Venezuelan equine encephalitis virus (VEEV), eastern equine encephalitis virus (EEEV), and western equine encephalitis virus (WEEV). In some embodiments, the SAM is derived from Venezuelan equine encephalitis virus (VEEV). In some embodiments, the virus comprises a modified viral genome. In some embodiments, the modified viral genome comprises a deletion of one or more genes encoding one or more viral structural proteins. In some embodiments, the modified viral genome is produced by replacing one or more viral structural proteins with any of the polynucleotides disclosed herein. In some embodiments, the modified viral genome is produced by replacing one or more viral structural proteins with a polynucleotide comprising a nucleic acid sequence that encodes the fusion polypeptide of any one of SEQ ID NOs: 527-528. In some embodiments, the modified viral genome is produced by replacing one or more viral structural proteins with a polynucleotide comprising a codon-optimized nucleic acid sequence that encodes the fusion polypeptide of any one of SEQ ID NOs: 527-528. In some embodiments, the modified viral genome is produced by replacing one or more viral structural proteins with a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 527-528, wherein percent identity is across the full length of the polypeptide of SEQ ID NOs: 527-528. In some embodiments, the SAM further comprises a promoter sequence. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529.

Further disclosed herein is a self-amplifying mRNA (SAM) comprising a modified viral genome. In some embodiments, the modified viral genome comprises a deletion of one or more genes encoding one or more viral structural proteins. In some embodiments, the modified viral genome is produced by replacing one or more viral structural proteins with any of the polynucleotides disclosed herein. In some embodiments, the modified viral genome is produced by replacing one or more viral structural proteins with a polynucleotide that encodes any of the polypeptides of SEQ ID NOs: 345-371, 373-377, 407-411, 422-423, 430-435, 527, and 528. In some embodiments, the modified viral genome is produced by replacing one or more viral structural proteins with a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 345-371, 373-377, 407-411, 422-423, 430-435, 527, and 528, wherein percent identity is across the full length of the polypeptide of SEQ ID NOs: 345-371, 373-377, 407-411, 422-423, 430-435, 527, and 528. In some embodiments, the modified viral genome is produced by replacing one or more viral structural proteins with a polynucleotide comprising a codon-optimized nucleic acid sequence that encodes any of the polypeptides of SEQ ID NOs: 345-371, 373-377, 407-411, 422-423, 430-435, 527, and 528. In some embodiments, the modified viral genome is produced by replacing one or more viral structural proteins with a polynucleotide encoding any of the polypeptides of SEQ ID NOs: 1-344. In some embodiments, the modified viral genome is produced by replacing one or more viral structural proteins with a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 1-344, wherein percent identity is across the full length of the polypeptide of 1-344. In some embodiments, the modified viral genome is produced by replacing one or more viral structural proteins with a polynucleotide comprising a codon-optimized nucleic acid sequence that encodes any one of the polypeptides of SEQ ID NOs: 1-344.

In some embodiments, the SAM is derived from a virus. In some embodiments, the virus is replication deficient. In some embodiments, the virus is an RNA virus. In some embodiments, the RNA virus is positive-sense single-stranded RNA virus. In some embodiments, the virus is selected from an alphavirus, flavivirus, nidovirus, nodamura virus, and picornavirus. In some embodiments, the alphavirus is selected from an Old World (OW) alphavirus and New World (NW) alpha virus. In some embodiments, the OW alphavirus is selected from Chikunguyna virus (CHIKV), Ross River virus (RRV), Semliki Forest virus (SFV), and Sindbis virus (SINV). In some embodiments, the NW alphavirus is selected from Venezuelan equine encephalitis virus (VEEV), eastern equine encephalitis virus (EEEV), and western equine encephalitis virus (WEEV). In some embodiments, the SAM is derived from Venezuelan equine encephalitis virus (VEEV). In some embodiments, the virus comprises a modified viral genome. In some embodiments, the modified viral genome comprises a deletion of one or more genes encoding one or more viral structural proteins. In some embodiments, the modified viral genome is produced by replacing one or more viral structural proteins with any of the polynucleotides disclosed herein. In some embodiments, the SAM further comprises a promoter sequence. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529.

Further disclosed herein are self-amplifying RNAs (SAMs or saRNAs) comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (e) a fourth polynucleotide sequence comprising nucleotides 10667 to 10952 of SEQ ID NO: 522; and (f) a polyA region. In some embodiments, the third polynucleotide is operably linked to and under the control of the promoter sequence. In some embodiments, the promoter sequence is a constitutive promoter. In some embodiments, the promoter is selected from a CMV promoter, a CAG promoter, an EF1a promoter, and a 26S sub-genomic promoter. In some embodiments, the promoter comprises or consists of any of the promoter sequences of SEQ ID NOs: 529-530. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529.

Further disclosed herein are self-amplifying RNAs (SAMs or saRNAs) comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 10667 to 10952 of SEQ ID NO: 522; and (f) a polyA region. In some embodiments, the third polynucleotide sequence encodes two or more polypeptide segments. In some embodiments, the third polynucleotide sequence encodes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344. In some embodiments, one or more of the polypeptide segments is abutted or fused to an adjacent polypeptide segment. In some embodiments, one or more of the polypeptide segments is joined to an adjacent polypeptide segment by one or more peptide linkers. In some embodiments, the one or more peptide linkers is selected from one or more of a polyalanine linker, a polyglycine linker, a cleavable linker, a flexible linker, a rigid linker, a Nef linking sequence, and combinations thereof. In some embodiments, the polyalanine linker comprises or consists of 2 or 3 contiguous alanine residues, e.g. AA, AAA, AAY or AAX, wherein X is any amino acid (e.g. A, C, D, E, F, G, H, I, K, L, M, N, P, Q, R, S, T, V, Y). In some embodiments, the flexible linker or polyglycine linker comprises or consists of GG, GGS, GSG or GGGS (SEQ ID NO: 421). In some embodiments, the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof. In some embodiments, the cleavable linker comprises or consists of a furin recognition/cleavage site selected from the group consisting of RAKR (SEQ ID NO: 381), REKR (SEQ ID NO: 382) and RRKR (SEQ ID NO: 383). In some embodiments, the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388). In some embodiments, the cleavable linker comprises or consists of the amino acid sequence of ATNFSLLKQAGDVEENPGP (SEQ ID NO: 384), ID NO: 385), APVKQTLNFDLLKLAGDVESNPGP (SEQ RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388). In some embodiments, the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), wherein percent identity is across the full length of SEQ ID NOs: 389-391. In some embodiments, the Nef linking sequence comprises or consists of an amino acid sequence selected from VHAGPIA (SEQ ID NO: 389), VHAGPVA (SEQ ID NO: 390) and GALDI (SEQ ID NO: 391). In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529.

Further disclosed herein are expression cassettes, expression vectors, or viral expression vectors comprising a polynucleotide comprising any one of the HIV immunogen nucleic acid sequences of SEQ ID NOs: 524-526, wherein the polynucleotide is operably linked to one or more regulatory sequences. Further disclosed herein are expression cassettes, expression vectors, or viral expression vectors comprising a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the HIV immunogen nucleic acid sequences of SEQ ID NOs: 524-526, wherein percent identity is across the full length of the HIV immunogen nucleic acid sequence of SEQ ID NOs: 524-526, and wherein the polynucleotide is operably linked to one or more regulatory sequences. Further disclosed are expression cassettes, expression vectors, or viral expression vectors comprising a polynucleotide comprising a codon-optimized nucleic acid sequence of any one of the HIV immunogen nucleic acid sequences of SEQ ID NOs: 524-526, wherein the polynucleotide is operably linked to one or more regulatory sequences. In some embodiments, the one or more regulatory sequences comprises a promoter sequence. In some embodiments, the promoter sequence is a constitutive promoter. In some embodiments, the promoter is selected from a CMV promoter, a CAG promoter, an EF1a promoter, and a 26S sub-genomic promoter. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529. In some embodiments, the promoter sequence comprises or consists of a polynucleotide sequence of SEQ ID NO: 530. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 530. In some embodiments, the promoter sequence comprises or consists of a codon-optimized nucleic acid sequence of the nucleic acid sequence of SEQ ID NO: 530.

Further disclosed herein are expression cassettes, expression vectors, or viral expression vectors comprising a polynucleotide comprising an HIV immunogen nucleic acid sequence that encodes any one of the HIV immunogen amino acid sequences of SEQ ID NOs: 527-528, wherein the polynucleotide is operably linked to one or more regulatory sequences. Further disclosed herein are expression cassettes, expression vectors, or viral expression vectors comprising a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 527-528, wherein percent identity is across the full length of the nucleic acid sequence of the polypeptide encoding any one of the HIV immunogen amino acid sequence of SEQ ID NOs: 527-528, and wherein the polynucleotide is operably linked to one or more regulatory sequences. Further disclosed herein are expression cassettes, expression vectors, or viral expression vectors comprising a polynucleotide comprising a codon-optimized nucleic acid sequence of an HIV immunogen nucleic acid sequence that encodes any one of the HIV immunogen amino acid sequences of SEQ ID NOs: 527-528, wherein the polynucleotide is operably linked to one or more regulatory sequences.

Further disclosed herein are expression cassettes, expression vectors, or viral expression vectors comprising a polynucleotide comprising an HIV immunogen nucleic acid sequence that encodes any one of the HIV immunogen amino acid sequences of SEQ ID NOs: 1-344, wherein the polynucleotide is operably linked to one or more regulatory sequences. Further disclosed herein are expression cassettes, expression vectors, or viral expression vectors comprising a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 1-344, wherein percent identity is across the full length of the nucleic acid sequence of the polypeptide encoding any one of the HIV immunogen amino acid sequence of SEQ ID NOs: 1-344, and wherein the polynucleotide is operably linked to one or more regulatory sequences. Further disclosed herein are expression cassettes, expression vectors, or viral expression vectors comprising a polynucleotide comprising a codon-optimized nucleic acid sequence of an HIV immunogen nucleic acid sequence that encodes any one of the HIV immunogen amino acid sequences of SEQ ID NOs: 1-344, wherein the polynucleotide is operably linked to one or more regulatory sequences.

Further disclosed herein are expression cassettes, expression vectors, or viral expression vectors comprising a polynucleotide comprising an HIV immunogen nucleic acid sequence that encodes any one of the HIV immunogen amino acid sequences of SEQ ID NOs: 345-371, 373-377, 407-411, 422-423, and 430-435, wherein the polynucleotide is operably linked to one or more regulatory sequences. Further disclosed herein are expression cassettes, expression vectors, or viral expression vectors comprising a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435, and wherein the polynucleotide is operably linked to one or more regulatory sequences. Further disclosed herein are expression cassettes, expression vectors, or viral expression vectors comprising a polynucleotide comprising a codon-optimized nucleic acid sequence of an HIV immunogen nucleic acid sequence that encodes any one of the HIV immunogen amino acid sequences of SEQ ID NOs: 345-371, 373-377, 407-411, 422-423, and 430-435, wherein the polynucleotide is operably linked to one or more regulatory sequences.

Further disclosed herein is an expression vector comprising: (a) a first polynucleotide sequence comprising nucleotides 1 to 593 of SEQ ID NO: 520; (b) a promoter sequence; (b) a second polynucleotide sequence comprising nucleotides 1196 to 1255 of SEQ ID NO: 520; (c) a third polynucleotide sequence encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (d) a fourth polynucleotide sequence comprising nucleotides 4352 to 4395 of SEQ ID NO: 520; (e) a polyA sequence; and (f) a fifth polynucleotide sequence comprising nucleotides 4620 to 32350 of SEQ ID NO: 520.

Further disclosed herein is an expression vector comprising: (a) a first polynucleotide sequence comprising nucleotides 1 to 593 of SEQ ID NO: 520; (b) a promoter sequence; (c) a second polynucleotide sequence comprising nucleotides 1196 to 1255 of SEQ ID NO: 520; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 4352 to 4395 of SEQ ID NO: 520; (f) a polyA sequence; and (g) a fifth polynucleotide sequence comprising nucleotides 4620 to 32350 of SEQ ID NO: 520. In some embodiments, the third polynucleotide sequence encodes two or more polypeptide segments. In some embodiments, the third polynucleotide sequence encodes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344. In some embodiments, one or more of the polypeptide segments is abutted or fused to an adjacent polypeptide segment. In some embodiments, one or more of the polypeptide segments is joined to an adjacent polypeptide segment by one or more peptide linkers. In some embodiments, the one or more peptide linkers is selected from one or more of a polyalanine linker, a polyglycine linker, a cleavable linker, a flexible linker, a rigid linker, a Nef linking sequence, and combinations thereof. In some embodiments, the polyalanine linker comprises or consists of 2 or 3 contiguous alanine residues, e.g. AA, AAA, AAY or AAX, wherein X is any amino acid (e.g. A, C, D, E, F, G, H, I, K, L, M, N, P, Q, R, S, T, V, Y). In some embodiments, the flexible linker or polyglycine linker comprises or consists of GG, GGS, GSG or GGGS (SEQ ID NO: 421). In some embodiments, the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof. In some embodiments, the cleavable linker comprises or consists of a furin recognition/cleavage site selected from the group consisting of RAKR (SEQ ID NO: 381), REKR (SEQ ID NO: 382) and RRKR (SEQ ID NO: 383). In some embodiments, the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388), wherein percent identity is across the full length of SEQ ID NOs: 384-388. In some embodiments, the cleavable linker comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388). In some embodiments, the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), wherein percent identity is across the full length of SEQ ID NOs: 389-391. In some embodiments, the Nef linking sequence comprises or consists of an amino acid sequence selected from VHAGPIA (SEQ ID NO: 389), VHAGPVA (SEQ ID NO: 390) and GALDI (SEQ ID NO: 391). In some embodiments, the promoter sequence is a constitutive promoter. In some embodiments, the promoter is selected from a CMV promoter, a CAG promoter, an EF1a promoter, and a 26S sub-genomic promoter. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529. In some embodiments, the promoter sequence comprises or consists of a polynucleotide sequence of SEQ ID NO: 530. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 530. In some embodiments, the promoter sequence comprises or consists of a codon-optimized nucleic acid sequence of the nucleic acid sequence of SEQ ID NO: 530.

Further disclosed herein are viral expression vectors comprising the nucleic acid sequence of any one of SEQ ID NOs: 520-521. Further disclosed herein are viral expression vectors comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 520-521. Further disclosed herein are viral expression vectors comprising a codon-optimized nucleic acid sequence of the nucleic acid sequence of any one of SEQ ID NOs: 520-521.

Further disclosed herein are lipid nanoparticles (LNPs), polymeric nanoparticles (PNPs), nanoemulsions, or viral vectors comprising (a) a polynucleotide comprising any one of the nucleic acid sequences of SEQ ID NOs: 522-523; or (b) a SAM, expression cassette, expression vector, or viral vector comprising such polynucleotide. Further disclosed herein are lipid nanoparticles (LNPs), polymeric nanoparticles (PNPs), nanoemulsions, or viral vectors comprising (a) a polynucleotide that comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523; or (b) a SAM, expression cassette, expression vector, or viral vector comprising such polynucleotide. Further disclosed herein are lipid nanoparticles (LNPs), polymeric nanoparticles (PNPs), nanoemulsions, or viral vectors comprising (a) a polynucleotide comprising a codon-optimized nucleic acid sequence of any one of the nucleic acid sequences of SEQ ID NOs: 522-523; or (b) a SAM, expression cassette, expression vector, or viral vector comprising such polynucleotide. In some embodiments, the LNP comprises or is synthesized from an ionizable lipid, phospholipid, cholesterol, PEGylated lipid, or any combination thereof. In some embodiments, the LNP comprises or is synthesized from cationic lipid 1,2-dioleoyl-3-timethylammonium-propane (DOTAP). In some embodiments, the PNP comprises or is synthesized from non-degradable polymers, degradable polymers, natural materials, synthetic materials, monomers, dendrimers, or any combination thereof. In some embodiments, the PNP comprises or is synthesized from poly(ethylene glycol) (PEG), poly(dimethylsiloxane) (PDMS), polyethyleneimine (PEI), poly(amidoamine) (PAMAM), poly(dimethylaminoethyl) acrylate (pDMAEA), orthenine-derived dendrimers, or any combination thereof. In some embodiments, the PNP comprises or is synthesized from mannosylated-PEI polyplexes. In some embodiments, the PNP comprises or is synthesized from a bioreducible, linear, cationic polymer (pABOL). In some embodiments, the nanoemulsion is a water-in-oil emulsion. In some embodiments, the nanoemulsion comprises squalene, sorbitan trioleate, polysorbate 80, DOTAP, or any combination thereof.

Further disclosed herein are lipid nanoparticles (LNPs), polymeric nanoparticles (PNPs), nanoemulsions, or viral vectors comprising (a) a polynucleotide comprising any one of the HIV immunogen nucleic acid sequences of SEQ ID NOs: 524-526; or (b) a SAM, expression cassette, expression vector, or viral vector comprising such polynucleotide. Further disclosed herein are lipid nanoparticles (LNPs), polymeric nanoparticles (PNPs), nanoemulsions, or viral vectors comprising (a) a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the HIV immunogen nucleic acid sequences of SEQ ID NOs: 524-526, wherein percent identity is across the full length of the HIV immunogen nucleic acid sequence of SEQ ID NOs: 524-526; or (b) a SAM, expression cassette, expression vector, or viral vector comprising such polynucleotide. Further disclosed herein are lipid nanoparticles (LNPs), polymeric nanoparticles (PNPs), nanoemulsions, or viral vectors comprising (a) a polynucleotide comprising a codon-optimized nucleic acid sequence of any one of the HIV immunogen nucleic acid sequences of SEQ ID NOs: 524-526; or (b) a SAM, expression cassette, expression vector, or viral vector comprising such polynucleotide. In some embodiments, the LNP comprises or is synthesized from an ionizable lipid, phospholipid, cholesterol, PEGylated lipid, or any combination thereof. In some embodiments, the LNP comprises or is synthesized from cationic lipid 1,2-dioleoyl-3-timethylammonium-propane (DOTAP). In some embodiments, the PNP comprises or is synthesized from non-degradable polymers, degradable polymers, natural materials, synthetic materials, monomers, dendrimers, or any combination thereof. In some embodiments, the PNP comprises or is synthesized from poly(ethylene glycol) (PEG), poly(dimethylsiloxane) (PDMS), polyethyleneimine (PEI), poly(amidoamine) (PAMAM), poly(dimethylaminoethyl) acrylate (pDMAEA), orthenine-derived dendrimers, or any combination thereof. In some embodiments, the PNP comprises or is synthesized from mannosylated-PEI polyplexes. In some embodiments, the PNP comprises or is synthesized from a bioreducible, linear, cationic polymer (pABOL). In some embodiments, the nanoemulsion is a water-in-oil emulsion. In some embodiments, the nanoemulsion comprises squalene, sorbitan trioleate, polysorbate 80, DOTAP, or any combination thereof. In some embodiments, the expression vector is a plasmid vector, a bacterial vector or a viral expression vector. In some embodiments, the expression vector is a viral expression vector. In some embodiments, the viral expression vector is a DNA virus or an RNA virus. In some embodiments, the viral expression vector is replication defective, replication deficient, replication attenuated or replication competent. In some embodiments, the viral expression vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus. In some embodiments, the viral expression vector is from a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus). In some embodiments, the viral expression vector is an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV). In some embodiments, the viral expression vector is an arenavirus vector selected from Lymphocytic choriomeningitis mammarenavirus (LCMV) or Cali mammarenavirus (a.k.a. Pichinde mammarenavirus or Pichinde arenavirus). In some embodiments, the arenavirus vector comprises a bi-segmented genome. In some embodiments, the arenavirus vector comprises a tri-segmented genome. In some embodiments, the viral expression vector is a human adenovirus or a simian adenovirus. In some embodiments, the simian adenovirus is selected from a chimpanzee adenovirus, a gorilla adenovirus, and a rhesus adenovirus. In some embodiments, the viral expression vector is an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (ChAd), gorilla adenovirus, and rhesus adenovirus. In some embodiments, the ChAd is selected from ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), and ChAd147 (AdC147). In some embodiments, the ChAd is ChAd68. In some embodiments, the gorilla adenovirus is selected from GC44, GC45, and GC46. In some embodiments, the rhesus adenovirus (RhAd) is selected from RhAd51, RhAd52, RhAd53, RhAd54, RhAd55, RhAd56, RhAd57, RhAd58, RhAd59, RhAd60, RhAd61, RhAd62, RhAd63, RhAd64, RhAd65 and RhAd66. In some embodiments, the human adenovirus or simian adenovirus comprises a modification of one or more adenoviral genes. In some embodiments, the one or more adenoviral genes are selected from E1, E3, and E4ORF2-4. In some embodiments, the modification comprises a deletion, substitution, or insertion of one or more nucleotides into the one or more adenoviral genes. In some embodiments, the human adenovirus or simian adenovirus comprises a deletion of at least a portion of the E1 region. In some embodiments, the polynucleotide is inserted into the deletion of at least a portion of the E1 region. In some embodiments, the polynucleotide is operably linked to and under the control of a constitutive promoter. In some embodiments, the promoter is selected from a CMV promoter, a CAG promoter, an EF1a promoter, and a 26S sub-genomic promoter. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529. In some embodiments, the promoter sequence comprises or consists of a polynucleotide sequence of SEQ ID NO: 530. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 530. In some embodiments, the promoter sequence comprises or consists of a codon-optimized nucleic acid sequence of the nucleic acid sequence of SEQ ID NO: 530.

Further disclosed herein are lipid nanoparticles (LNPs), polymeric nanoparticles (PNPs), nanoemulsions, or viral vectors comprising (a) a polynucleotide comprising an HIV immunogen nucleic acid sequence that encodes any one of the HIV immunogen amino acid sequences of SEQ ID NOs: 527-528; or (b) a SAM, expression cassette, expression vector, or viral vector comprising such polynucleotide. Further disclosed herein are lipid nanoparticles (LNPs), polymeric nanoparticles (PNPs), nanoemulsions, or viral vectors comprising (a) a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 527-528, wherein percent identity is across the full length of the nucleic acid sequence of the polypeptide encoding any one of the HIV immunogen amino acid sequence of SEQ ID NOs: 527-528; or (b) a SAM, expression cassette, expression vector, or viral vector comprising such polynucleotide. Further disclosed herein are lipid nanoparticles (LNPs), polymeric nanoparticles (PNPs), nanoemulsions, or viral vectors comprising (a) a polynucleotide comprising a codon-optimized nucleic acid sequence of an HIV immunogen nucleic acid sequence that encodes any one of the HIV immunogen amino acid sequences of SEQ ID NOs: 527-528; or (b) a SAM, expression cassette, expression vector, or viral vector comprising such polynucleotide. In some embodiments, the LNP comprises or is synthesized from an ionizable lipid, phospholipid, cholesterol, PEGylated lipid, or any combination thereof. In some embodiments, the LNP comprises or is synthesized from cationic lipid 1,2-dioleoyl-3-timethylammonium-propane (DOTAP). In some embodiments, the PNP comprises or is synthesized from non-degradable polymers, degradable polymers, natural materials, synthetic materials, monomers, dendrimers, or any combination thereof. In some embodiments, the PNP comprises or is synthesized from poly(ethylene glycol) (PEG), poly(dimethylsiloxane) (PDMS), polyethyleneimine (PEI), poly(amidoamine) (PAMAM), poly(dimethylaminoethyl) acrylate (pDMAEA), orthenine-derived dendrimers, or any combination thereof. In some embodiments, the PNP comprises or is synthesized from mannosylated-PEI polyplexes. In some embodiments, the PNP comprises or is synthesized from a bioreducible, linear, cationic polymer (pABOL). In some embodiments, the nanoemulsion is a water-in-oil emulsion. In some embodiments, the nanoemulsion comprises squalene, sorbitan trioleate, polysorbate 80, DOTAP, or any combination thereof. In some embodiments, the expression vector is a plasmid vector, a bacterial vector or a viral expression vector. In some embodiments, the expression vector is a viral expression vector. In some embodiments, the viral expression vector is a DNA virus or an RNA virus. In some embodiments, the viral expression vector is replication defective, replication deficient, replication attenuated or replication competent. In some embodiments, the viral expression vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus. In some embodiments, the viral expression vector is from a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus). In some embodiments, the viral expression vector is an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV). In some embodiments, the viral expression vector is an arenavirus vector selected from Lymphocytic choriomeningitis mammarenavirus (LCMV) or Cali mammarenavirus (a.k.a. Pichinde mammarenavirus or Pichinde arenavirus). In some embodiments, the arenavirus vector comprises a bi-segmented genome. In some embodiments, the arenavirus vector comprises a tri-segmented genome. In some embodiments, the viral expression vector is a human adenovirus or a simian adenovirus. In some embodiments, the simian adenovirus is selected from a chimpanzee adenovirus, a gorilla adenovirus, and a rhesus adenovirus. In some embodiments, the viral expression vector is an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (ChAd), gorilla adenovirus, and rhesus adenovirus. In some embodiments, the ChAd is selected from ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), and ChAd147 (AdC147). In some embodiments, the ChAd is ChAd68. In some embodiments, the gorilla adenovirus is selected from GC44, GC45, and GC46. In some embodiments, the rhesus adenovirus (RhAd) is selected from RhAd51, RhAd52, RhAd53, RhAd54, RhAd55, RhAd56, RhAd57, RhAd58, RhAd59, RhAd60, RhAd61, RhAd62, RhAd63, RhAd64, RhAd65 and RhAd66. In some embodiments, the human adenovirus or simian adenovirus comprises a modification of one or more adenoviral genes. In some embodiments, the one or more adenoviral genes are selected from E1, E3, and E4ORF2-4. In some embodiments, the modification comprises a deletion, substitution, or insertion of one or more nucleotides into the one or more adenoviral genes. In some embodiments, the human adenovirus or simian adenovirus comprises a deletion of at least a portion of the E1 region. In some embodiments, the polynucleotide is inserted into the deletion of at least a portion of the E1 region. In some embodiments, the polynucleotide is operably linked to and under the control of a constitutive promoter. In some embodiments, the promoter is selected from a CMV promoter, a CAG promoter, an EF1a promoter, and a 26S sub-genomic promoter. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529. In some embodiments, the promoter sequence comprises or consists of a polynucleotide sequence of SEQ ID NO: 530. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 530. In some embodiments, the promoter sequence comprises or consists of a codon-optimized nucleic acid sequence of the nucleic acid sequence of SEQ ID NO: 530.

Further disclosed herein are lipid nanoparticles (LNPs), polymeric nanoparticles (PNPs), nanoemulsions, or viral vectors comprising a modified viral genome. In some embodiments, the modified viral genome comprises a deletion of one or more genes encoding one or more viral structural proteins. In some embodiments, the modified viral genome is produced by replacing one or more viral structural proteins with any of the polynucleotides disclosed herein. In some embodiments, the modified viral genome is produced by replacing one or more viral structural proteins with a polynucleotide that encodes any of the polypeptides of SEQ ID NOs: 345-371, 373-377, 407-411, 422-423, 430-435, 527, and 528. In some embodiments, the modified viral genome is produced by replacing one or more viral structural proteins with a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 345-371, 373-377, 407-411, 422-423, 430-435, 527, and 528, wherein percent identity is across the full length of the polypeptide of SEQ ID NOs: 345-371, 373-377, 407-411, 422-423, 430-435, 527, and 528. In some embodiments, the modified viral genome is produced by replacing one or more viral structural proteins with a polynucleotide comprising a codon-optimized nucleic acid sequence that encodes any of the polypeptides of SEQ ID NOs: 345-371, 373-377, 407-411, 422-423, 430-435, 527, and 528. In some embodiments, the modified viral genome is produced by replacing one or more viral structural proteins with a polynucleotide encoding any of the polypeptides of SEQ ID NOs: 1-344. In some embodiments, the modified viral genome is produced by replacing one or more viral structural proteins with a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 1-344, wherein percent identity is across the full length of the polypeptide of 1-344. In some embodiments, the modified viral genome is produced by replacing one or more viral structural proteins with a polynucleotide comprising a codon-optimized nucleic acid sequence that encodes any one of the polypeptides of SEQ ID NOs: 1-344. In some embodiments, the LNP comprises or is synthesized from an ionizable lipid, phospholipid, cholesterol, PEGylated lipid, or any combination thereof. In some embodiments, the LNP comprises or is synthesized from cationic lipid 1,2-dioleoyl-3-timethylammonium-propane (DOTAP). In some embodiments, the PNP comprises or is synthesized from non-degradable polymers, degradable polymers, natural materials, synthetic materials, monomers, dendrimers, or any combination thereof. In some embodiments, the PNP comprises or is synthesized from poly(ethylene glycol) (PEG), poly(dimethylsiloxane) (PDMS), polyethyleneimine (PEI), poly(amidoamine) (PAMAM), poly(dimethylaminoethyl) acrylate (pDMAEA), orthenine-derived dendrimers, or any combination thereof. In some embodiments, the PNP comprises or is synthesized from mannosylated-PEI polyplexes. In some embodiments, the PNP comprises or is synthesized from a bioreducible, linear, cationic polymer (pABOL). In some embodiments, the nanoemulsion is a water-in-oil emulsion. In some embodiments, the nanoemulsion comprises squalene, sorbitan trioleate, polysorbate 80, DOTAP, or any combination thereof. In some embodiments, the expression vector is a plasmid vector, a bacterial vector or a viral expression vector. In some embodiments, the expression vector is a viral expression vector. In some embodiments, the viral expression vector is a DNA virus or an RNA virus. In some embodiments, the viral expression vector is replication defective, replication deficient, replication attenuated or replication competent. In some embodiments, the viral expression vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus. In some embodiments, the viral expression vector is from a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus). In some embodiments, the viral expression vector is an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV). In some embodiments, the viral expression vector is an arenavirus vector selected from Lymphocytic choriomeningitis mammarenavirus (LCMV) or Cali mammarenavirus (a.k.a. Pichinde mammarenavirus or Pichinde arenavirus). In some embodiments, the arenavirus vector comprises a bi-segmented genome. In some embodiments, the arenavirus vector comprises a tri-segmented genome. In some embodiments, the viral expression vector is a human adenovirus or a simian adenovirus. In some embodiments, the simian adenovirus is selected from a chimpanzee adenovirus, a gorilla adenovirus, and a rhesus adenovirus. In some embodiments, the viral expression vector is an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (ChAd), gorilla adenovirus, and rhesus adenovirus. In some embodiments, the ChAd is selected from ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), and ChAd147 (AdC147). In some embodiments, the ChAd is ChAd68. In some embodiments, the gorilla adenovirus is selected from GC44, GC45, and GC46. In some embodiments, the rhesus adenovirus (RhAd) is selected from RhAd51, RhAd52, RhAd53, RhAd54, RhAd55, RhAd56, RhAd57, RhAd58, RhAd59, RhAd60, RhAd61, RhAd62, RhAd63, RhAd64, RhAd65 and RhAd66. In some embodiments, the human adenovirus or simian adenovirus comprises a modification of one or more adenoviral genes. In some embodiments, the one or more adenoviral genes are selected from E1, E3, and E4ORF2-4. In some embodiments, the modification comprises a deletion, substitution, or insertion of one or more nucleotides into the one or more adenoviral genes. In some embodiments, the human adenovirus or simian adenovirus comprises a deletion of at least a portion of the E1 region. In some embodiments, the polynucleotide is inserted into the deletion of at least a portion of the E1 region. In some embodiments, the polynucleotide is operably linked to and under the control of a constitutive promoter. In some embodiments, the promoter is selected from a CMV promoter, a CAG promoter, an EF1a promoter, and a 26S sub-genomic promoter. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529. In some embodiments, the promoter sequence comprises or consists of a polynucleotide sequence of SEQ ID NO: 530. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 530. In some embodiments, the promoter sequence comprises or consists of a codon-optimized nucleic acid sequence of the nucleic acid sequence of SEQ ID NO: 530.

Further disclosed herein are lipid nanoparticles (LNPs), polymeric nanoparticles (PNPs), nanoemulsions, or viral vectors comprising (a) a polynucleotide comprising an HIV immunogen nucleic acid sequence that encodes any one of the HIV immunogen amino acid sequences of SEQ ID NOs: 1-344, wherein the polynucleotide is operably linked to one or more regulatory sequences; or (b) an expression cassette, expression vector, or viral expression vector comprising such polynucleotide. Further disclosed herein are lipid nanoparticles (LNPs), polymeric nanoparticles (PNPs), nanoemulsions, or viral vectors comprising (a) a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 1-344, wherein percent identity is across the full length of the nucleic acid sequence of the polypeptide encoding any one of the HIV immunogen amino acid sequence of SEQ ID NOs: 1-344, and wherein the polynucleotide is operably linked to one or more regulatory sequences; or (b) an expression cassette, expression vector, or viral expression vector comprising such polynucleotide. Further disclosed herein are lipid nanoparticles (LNPs), polymeric nanoparticles (PNPs), nanoemulsions, or viral vectors comprising (a) a polynucleotide comprising a codon-optimized nucleic acid sequence of an HIV immunogen nucleic acid sequence that encodes any one of the HIV immunogen amino acid sequences of SEQ ID NOs: 1-344, wherein the polynucleotide is operably linked to one or more regulatory sequences; or (b) an expression cassette, expression vector, or viral expression vector comprising such polynucleotide. In some embodiments, the LNP comprises or is synthesized from an ionizable lipid, phospholipid, cholesterol, PEGylated lipid, or any combination thereof. In some embodiments, the LNP comprises or is synthesized from cationic lipid 1,2-dioleoyl-3-timethylammonium-propane (DOTAP). In some embodiments, the PNP comprises or is synthesized from non-degradable polymers, degradable polymers, natural materials, synthetic materials, monomers, dendrimers, or any combination thereof. In some embodiments, the PNP comprises or is synthesized from poly(ethylene glycol) (PEG), poly(dimethylsiloxane) (PDMS), polyethyleneimine (PEI), poly(amidoamine) (PAMAM), poly(dimethylaminoethyl) acrylate (pDMAEA), orthenine-derived dendrimers, or any combination thereof. In some embodiments, the PNP comprises or is synthesized from mannosylated-PEI polyplexes. In some embodiments, the PNP comprises or is synthesized from a bioreducible, linear, cationic polymer (pABOL). In some embodiments, the nanoemulsion is a water-in-oil emulsion. In some embodiments, the nanoemulsion comprises squalene, sorbitan trioleate, polysorbate 80, DOTAP, or any combination thereof. In some embodiments, the expression vector is a plasmid vector, a bacterial vector or a viral expression vector. In some embodiments, the expression vector is a viral expression vector. In some embodiments, the viral expression vector is a DNA virus or an RNA virus. In some embodiments, the viral expression vector is replication defective, replication deficient, replication attenuated or replication competent. In some embodiments, the viral expression vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus. In some embodiments, the viral expression vector is from a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus). In some embodiments, the viral expression vector is an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV). In some embodiments, the viral expression vector is an arenavirus vector selected from Lymphocytic choriomeningitis mammarenavirus (LCMV) or Cali mammarenavirus (a.k.a. Pichinde mammarenavirus or Pichinde arenavirus). In some embodiments, the arenavirus vector comprises a bi-segmented genome. In some embodiments, the arenavirus vector comprises a tri-segmented genome. In some embodiments, the viral expression vector is a human adenovirus or a simian adenovirus. In some embodiments, the simian adenovirus is selected from a chimpanzee adenovirus, a gorilla adenovirus, and a rhesus adenovirus. In some embodiments, the viral expression vector is an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (ChAd), gorilla adenovirus, and rhesus adenovirus. In some embodiments, the ChAd is selected from ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), and ChAd147 (AdC147). In some embodiments, the ChAd is ChAd68. In some embodiments, the gorilla adenovirus is selected from GC44, GC45, and GC46. In some embodiments, the rhesus adenovirus (RhAd) is selected from RhAd51, RhAd52, RhAd53, RhAd54, RhAd55, RhAd56, RhAd57, RhAd58, RhAd59, RhAd60, RhAd61, RhAd62, RhAd63, RhAd64, RhAd65 and RhAd66. In some embodiments, the human adenovirus or simian adenovirus comprises a modification of one or more adenoviral genes. In some embodiments, the one or more adenoviral genes are selected from E1, E3, and E4ORF2-4. In some embodiments, the modification comprises a deletion, substitution, or insertion of one or more nucleotides into the one or more adenoviral genes. In some embodiments, the human adenovirus or simian adenovirus comprises a deletion of at least a portion of the E1 region. In some embodiments, the polynucleotide is inserted into the deletion of at least a portion of the E1 region. In some embodiments, the polynucleotide is operably linked to and under the control of a constitutive promoter. In some embodiments, the promoter is selected from a CMV promoter, a CAG promoter, an EF1a promoter, and a 26S sub-genomic promoter. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529. In some embodiments, the promoter sequence comprises or consists of a polynucleotide sequence of SEQ ID NO: 530. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 530. In some embodiments, the promoter sequence comprises or consists of a codon-optimized nucleic acid sequence of the nucleic acid sequence of SEQ ID NO: 530.

Further disclosed herein are lipid nanoparticles (LNPs), polymeric nanoparticles (PNPs), nanoemulsions, or viral vectors comprising (a) a polynucleotide comprising an HIV immunogen nucleic acid sequence that encodes any one of the HIV immunogen amino acid sequences of SEQ ID NOs: 345-371, 373-377, 407-411, 422-423, and 430-435, wherein the polynucleotide is operably linked to one or more regulatory sequences; or (b) an expression cassette, expression vector, or viral expression vector comprising such polynucleotide. Further disclosed herein are lipid nanoparticles (LNPs), polymeric nanoparticles (PNPs), nanoemulsions, or viral vectors comprising (a) a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435, and wherein the polynucleotide is operably linked to one or more regulatory sequences; or (b) an expression cassette, expression vector, or viral expression vector comprising such polynucleotide. Further disclosed herein are lipid nanoparticles (LNPs), polymeric nanoparticles (PNPs), nanoemulsions, or viral vectors comprising (a) a polynucleotide comprising a codon-optimized nucleic acid sequence of an HIV immunogen nucleic acid sequence that encodes any one of the HIV immunogen amino acid sequences of SEQ ID NOs: 345-371, 373-377, 407-411, 422-423, and 430-435, wherein the polynucleotide is operably linked to one or more regulatory sequences; or (b) an expression cassette, expression vector, or viral expression vector comprising such polynucleotide. In some embodiments, the LNP comprises or is synthesized from an ionizable lipid, phospholipid, cholesterol, PEGylated lipid, or any combination thereof. In some embodiments, the LNP comprises or is synthesized from cationic lipid 1,2-dioleoyl-3-timethylammonium-propane (DOTAP). In some embodiments, the PNP comprises or is synthesized from non-degradable polymers, degradable polymers, natural materials, synthetic materials, monomers, dendrimers, or any combination thereof. In some embodiments, the PNP comprises or is synthesized from poly(ethylene glycol) (PEG), poly(dimethylsiloxane) (PDMS), polyethyleneimine (PEI), poly(amidoamine) (PAMAM), poly(dimethylaminoethyl) acrylate (pDMAEA), orthenine-derived dendrimers, or any combination thereof. In some embodiments, the PNP comprises or is synthesized from mannosylated-PEI polyplexes. In some embodiments, the PNP comprises or is synthesized from a bioreducible, linear, cationic polymer (pABOL). In some embodiments, the nanoemulsion is a water-in-oil emulsion. In some embodiments, the nanoemulsion comprises squalene, sorbitan trioleate, polysorbate 80, DOTAP, or any combination thereof. In some embodiments, the expression vector is a plasmid vector, a bacterial vector or a viral expression vector. In some embodiments, the expression vector is a viral expression vector. In some embodiments, the viral expression vector is a DNA virus or an RNA virus. In some embodiments, the viral expression vector is replication defective, replication deficient, replication attenuated or replication competent. In some embodiments, the viral expression vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus. In some embodiments, the viral expression vector is from a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus). In some embodiments, the viral expression vector is an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV). In some embodiments, the viral expression vector is an arenavirus vector selected from Lymphocytic choriomeningitis mammarenavirus (LCMV) or Cali mammarenavirus (a.k.a. Pichinde mammarenavirus or Pichinde arenavirus). In some embodiments, the arenavirus vector comprises a bi-segmented genome. In some embodiments, the arenavirus vector comprises a tri-segmented genome. In some embodiments, the viral expression vector is a human adenovirus or a simian adenovirus. In some embodiments, the simian adenovirus is selected from a chimpanzee adenovirus, a gorilla adenovirus, and a rhesus adenovirus. In some embodiments, the viral expression vector is an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (ChAd), gorilla adenovirus, and rhesus adenovirus. In some embodiments, the ChAd is selected from ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), and ChAd147 (AdC147). In some embodiments, the ChAd is ChAd68. In some embodiments, the gorilla adenovirus is selected from GC44, GC45, and GC46. In some embodiments, the rhesus adenovirus (RhAd) is selected from RhAd51, RhAd52, RhAd53, RhAd54, RhAd55, RhAd56, RhAd57, RhAd58, RhAd59, RhAd60, RhAd61, RhAd62, RhAd63, RhAd64, RhAd65 and RhAd66. In some embodiments, the human adenovirus or simian adenovirus comprises a modification of one or more adenoviral genes. In some embodiments, the one or more adenoviral genes are selected from E1, E3, and E4ORF2-4. In some embodiments, the modification comprises a deletion, substitution, or insertion of one or more nucleotides into the one or more adenoviral genes. In some embodiments, the human adenovirus or simian adenovirus comprises a deletion of at least a portion of the E1 region. In some embodiments, the polynucleotide is inserted into the deletion of at least a portion of the E1 region. In some embodiments, the polynucleotide is operably linked to and under the control of a constitutive promoter. In some embodiments, the promoter is selected from a CMV promoter, a CAG promoter, an EF1a promoter, and a 26S sub-genomic promoter. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529. In some embodiments, the promoter sequence comprises or consists of a polynucleotide sequence of SEQ ID NO: 530. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 530. In some embodiments, the promoter sequence comprises or consists of a codon-optimized nucleic acid sequence of the nucleic acid sequence of SEQ ID NO: 530.

Further disclosed herein are lipid nanoparticles (LNPs), polymeric nanoparticles (PNPs), nanoemulsions, or viral vectors comprising a self-amplifying RNA (SAM) comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOS: 345-371, 373-377, 407-411, 422-423, 430-435, 527, and 528; (e) a fourth polynucleotide sequence comprising nucleotides 10667 to 10952 of SEQ ID NO: 522; and (f) a polyA region. In some embodiments, the third polynucleotide is operably linked to and under the control of the promoter sequence. In some embodiments, the promoter sequence is a constitutive promoter. In some embodiments, the promoter is selected from a CMV promoter, a CAG promoter, an EFla promoter, and a 26S sub-genomic promoter. In some embodiments, the promoter comprises or consists of any of the promoter sequences of SEQ ID NOs: 529-530. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529.

Further disclosed herein are lipid nanoparticles (LNPs), polymeric nanoparticles (PNPs), nanoemulsions, or viral vectors comprising a self-amplifying RNA (SAM) comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 10667 to 10952 of SEQ ID NO: 522; and (f) a polyA region. In some embodiments, the third polynucleotide sequence encodes two or more polypeptide segments. In some embodiments, the third polynucleotide sequence encodes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344. In some embodiments, one or more of the polypeptide segments is abutted or fused to an adjacent polypeptide segment. In some embodiments, one or more of the polypeptide segments is joined to an adjacent polypeptide segment by one or more peptide linkers. In some embodiments, the one or more peptide linkers is selected from one or more of a polyalanine linker, a polyglycine linker, a cleavable linker, a flexible linker, a rigid linker, a Nef linking sequence, and combinations thereof. In some embodiments, the polyalanine linker comprises or consists of 2 or 3 contiguous alanine residues, e.g. AA, AAA, AAY or AAX, wherein X is any amino acid (e.g. A, C, D, E, F, G, H, I, K, L, M, N, P, Q, R, S, T, V, Y). In some embodiments, the flexible linker or polyglycine linker comprises or consists of GG, GGS, GSG or GGGS (SEQ ID NO: 421). In some embodiments, the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof. In some embodiments, the cleavable linker comprises or consists of a furin recognition/cleavage site selected from the group consisting of RAKR (SEQ ID NO: 381), REKR (SEQ ID NO: 382) and RRKR (SEQ ID NO: 383). In some embodiments, the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388). In some embodiments, the cleavable linker comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388). In some embodiments, the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), wherein percent identity is across the full length of SEQ ID NOs: 389-391. In some embodiments, the Nef linking sequence comprises or consists of an amino acid sequence selected from VHAGPIA (SEQ ID NO: 389), VHAGPVA (SEQ ID NO: 390) and GALDI (SEQ ID NO: 391). In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529.

Further disclosed herein are viral vectors comprising an expression vector comprising: (a) a first polynucleotide sequence comprising nucleotides 1 to 593 of SEQ ID NO: 520; (b) a promoter sequence; (b) a second polynucleotide sequence comprising nucleotides 1196 to 1255 of SEQ ID NO: 520; (c) a third polynucleotide sequence encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (d) a fourth polynucleotide sequence comprising nucleotides 4352 to 4395 of SEQ ID NO: 520; (e) a polyA sequence; and (f) a fifth polynucleotide sequence comprising nucleotides 4620 to 32350 of SEQ ID NO: 520. In some embodiments, the promoter sequence comprises or consists of a polynucleotide sequence of SEQ ID NO: 530. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 530. In some embodiments, the promoter sequence comprises or consists of a codon-optimized nucleic acid sequence of the nucleic acid sequence of SEQ ID NO: 530.

Further disclosed herein are viral vectors comprising an expression vector comprising: (a) a first polynucleotide sequence comprising nucleotides 1 to 593 of SEQ ID NO: 520; (b) a promoter sequence; (c) a second polynucleotide sequence comprising nucleotides 1196 to 1255 of SEQ ID NO: 520; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 4352 to 4395 of SEQ ID NO: 520; (f) a polyA sequence; and (g) a fifth polynucleotide sequence comprising nucleotides 4620 to 32350 of SEQ ID NO: 520. In some embodiments, the third polynucleotide sequence encodes two or more polypeptide segments. In some embodiments, the third polynucleotide sequence encodes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344. In some embodiments, one or more of the polypeptide segments is abutted or fused to an adjacent polypeptide segment. In some embodiments, one or more of the polypeptide segments is joined to an adjacent polypeptide segment by one or more peptide linkers. In some embodiments, the one or more peptide linkers is selected from one or more of a polyalanine linker, a polyglycine linker, a cleavable linker, a flexible linker, a rigid linker, a Nef linking sequence, and combinations thereof. In some embodiments, the polyalanine linker comprises or consists of 2 or 3 contiguous alanine residues, e.g. AA, AAA, AAY or AAX, wherein X is any amino acid (e.g. A, C, D, E, F, G, H, I, K, L, M, N, P, Q, R, S, T, V, Y). In some embodiments, the flexible linker or polyglycine linker comprises or consists of GG, GGS, GSG or GGGS (SEQ ID NO: 421). In some embodiments, the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof. In some embodiments, the cleavable linker comprises or consists of a furin recognition/cleavage site selected from the group consisting of RAKR (SEQ ID NO: 381), REKR (SEQ ID NO: 382) and RRKR (SEQ ID NO: 383). In some embodiments, the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388), wherein percent identity is across the full length of SEQ ID NOs: 384-388. In some embodiments, the cleavable linker comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388). In some embodiments, the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), wherein percent identity is across the full length of SEQ ID NOs: 389-391. In some embodiments, the Nef linking sequence comprises or consists of an amino acid sequence selected from VHAGPIA (SEQ ID NO: 389), VHAGPVA (SEQ ID NO: 390) and GALDI (SEQ ID NO: 391). In some embodiments, the promoter sequence is a constitutive promoter. In some embodiments, the promoter is selected from a CMV promoter, a CAG promoter, an EF1a promoter, and a 26S sub-genomic promoter. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529. In some embodiments, the promoter sequence comprises or consists of a polynucleotide sequence of SEQ ID NO: 530. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 530. In some embodiments, the promoter sequence comprises or consists of a codon-optimized nucleic acid sequence of the nucleic acid sequence of SEQ ID NO: 530.

Further disclosed herein are viral vectors comprising a viral expression vector comprising the nucleic acid sequence of any one of SEQ ID NOs: 520-521. Further disclosed herein are viral vectors comprising a viral expression vector comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 520-521. Further disclosed herein are viral vectors comprising a viral expression vector comprising a codon-optimized nucleic acid sequence of the nucleic acid sequence of any one of SEQ ID NOS: 520-521.

Further disclosed herein are polypeptides comprising any of the HIV immunogen amino acid sequences of SEQ ID NOs: 527-528. Further disclosed herein are polypeptides comprising an amino acid sequence that is at least 80%, 85%, 86%, 87%, 88%, 89%, 90%, 91%, 92%, 93%, 94%, 95%, 96%, 97%, 98%, 99%, or 100% identical any of the HIV immunogen amino acid sequences of SEQ ID NOs: 527-528, wherein percent identity is across the full length of the HIV immunogen amino acid sequence of SEQ ID NOs: 527-528.

Further disclosed herein are compositions or kits comprising (a) a polynucleotide comprising any one of the nucleic acid sequences of SEQ ID NOs: 522-523; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. Further disclosed herein are compositions or kits comprising (a) a polynucleotide that comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. Further disclosed herein are compositions or kits comprising (a) a polynucleotide comprising a codon-optimized nucleic acid sequence of any one of the nucleic acid sequences of SEQ ID NOs: 522-523; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. In some embodiments, the LNP comprises or is synthesized from an ionizable lipid, phospholipid, cholesterol, PEGylated lipid, or any combination thereof. In some embodiments, the LNP comprises or is synthesized from cationic lipid 1,2-dioleoyl-3-timethylammonium-propane (DOTAP). In some embodiments, the PNP comprises or is synthesized from non-degradable polymers, degradable polymers, natural materials, synthetic materials, monomers, dendrimers, or any combination thereof. In some embodiments, the PNP comprises or is synthesized from poly(ethylene glycol) (PEG), poly(dimethylsiloxane) (PDMS), polyethyleneimine (PEI), poly(amidoamine) (PAMAM), poly(dimethylaminoethyl) acrylate (pDMAEA), orthenine-derived dendrimers, or any combination thereof. In some embodiments, the PNP comprises or is synthesized from mannosylated-PEI polyplexes. In some embodiments, the PNP comprises or is synthesized from a bioreducible, linear, cationic polymer (pABOL). In some embodiments, the nanoemulsion is a water-in-oil emulsion. In some embodiments, the nanoemulsion comprises squalene, sorbitan trioleate, polysorbate 80, DOTAP, or any combination thereof. In some embodiments, any of the compositions disclosed herein further comprise a pharmaceutically acceptable diluent, carrier or excipient. In some embodiments, any of the compositions or kits disclosed herein further comprise one or more of an adjuvant, an immunostimulator, a detergent, a micelle-forming agent, and an oil. In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein. In some embodiments, the cytokine selected from IL-2, IL-7, IL-12, IL-15, IL-18, IL-21, IFN-α, IFN-γ, GM-CSF, FLT3LG, and combinations and functional variants thereof. In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102). In some embodiments, the composition is formulated for administration via a route selected from the group consisting of intravenous, intramuscular, intradermal, subcutaneous and mucosal (e.g. buccal, intranasal, intrarectal, intravaginal). In some embodiments, the composition is formulated as a liquid. In some embodiments, the composition is lyophilized. In some embodiments, any of the compositions or kits disclosed herein further comprise one or more unitary doses of one or more additional therapeutic agents. In some embodiments, any of the compositions or kits disclosed herein further comprise GS 9620 (vesatolimod). In some embodiments, any of the compositions or kits disclosed herein further comprise lefitolimod (MGN-1703). In some embodiments, any of the compositions or kits disclosed herein further comprise a bnAb. In some embodiments, the bnAb is an HIV bnAb. In some embodiments, any of the compositions or kits disclosed herein further comprise (a) any of the SAMs or viral expression vectors disclosed herein; or (b) any of the LNPs, PNPs, nanoemulsions, or viral vectors comprising such SAMs or viral expression vectors disclosed herein. In some embodiments, the viral expression vector comprises a polynucleotide comprising (a) the nucleic sequence of any one of SEQ ID NOs: 520-521; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 520-521; or (c) a codon-optimized nucleic acid sequence of the nucleic sequence of any one of SEQ ID NOs: 520-521. In some embodiments, the SAM comprises a polynucleotide comprising (a) the nucleic sequence of any one of SEQ ID NOs: 522-523; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 522-523; or (c) a codon-optimized nucleic acid sequence of the nucleic sequence of any one of SEQ ID NOs: 522-523. In some embodiments, SAM or viral expression vector comprises a polynucleotide comprising (a) the nucleic sequence of any one of SEQ ID NOs: 524-526; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 524-526; or (c) a codon-optimized nucleic acid sequence of the nucleic sequence of any one of SEQ ID NOs: 524-526. In some embodiments, SAM or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 527-528, wherein percent identity is across the full length of the nucleic acid sequence of the polypeptide encoding any one of the HIV immunogen amino acid sequence of SEQ ID NOs: 527-528; or (c) a codon-optimized nucleic acid sequence of the nucleic sequence encoding the polypeptide of any one of SEQ ID NOs: 1-344. In some embodiments, SAM or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 1-344, wherein percent identity is across the full length of the nucleic acid sequence of the polypeptide encoding any one of the HIV immunogen amino acid sequence of SEQ ID NOs: 1-344; or (c) a codon-optimized nucleic acid sequence of the nucleic sequence encoding the polypeptide of any one of SEQ ID NOs: 1-344. In some embodiments, SAM or viral expression vector comprises a polynucleotide comprising (a) a nucleic sequence encoding the polypeptide of any one of SEQ ID NOs: 345-371, 373-377, 407-411, 422-423, and 430-435; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435; or (c) a codon-optimized nucleic acid sequence of the nucleic sequence encoding the polypeptide of any one of SEQ ID NOs: 345-371, 373-377, 407-411, 422-423, and 430-435.

Further disclosed herein are compositions or kits comprising (a) a polynucleotide comprising any one of the HIV immunogen nucleic acid sequences of SEQ ID NOs: 524-526; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. Further disclosed herein are compositions or kits (a) a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the HIV immunogen nucleic acid sequences of SEQ ID NOs: 524-526, wherein percent identity is across the full length of the HIV immunogen nucleic acid sequence of SEQ ID NOs: 524-526; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. Further disclosed herein are compositions or kits comprising (a) a polynucleotide comprising a codon-optimized nucleic acid sequence of any one of the HIV immunogen nucleic acid sequences of SEQ ID NOs: 524-526; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. In some embodiments, the LNP comprises or is synthesized from an ionizable lipid, phospholipid, cholesterol, PEGylated lipid, or any combination thereof. In some embodiments, the LNP comprises or is synthesized from cationic lipid 1,2-dioleoyl-3-timethylammonium-propane (DOTAP). In some embodiments, the PNP comprises or is synthesized from non-degradable polymers, degradable polymers, natural materials, synthetic materials, monomers, dendrimers, or any combination thereof. In some embodiments, the PNP comprises or is synthesized from poly(ethylene glycol) (PEG), poly(dimethylsiloxane) (PDMS), polyethyleneimine (PEI), poly(amidoamine) (PAMAM), poly(dimethylaminoethyl) acrylate (pDMAEA), orthenine-derived dendrimers, or any combination thereof. In some embodiments, the PNP comprises or is synthesized from mannosylated-PEI polyplexes. In some embodiments, the PNP comprises or is synthesized from a bioreducible, linear, cationic polymer (pABOL). In some embodiments, the nanoemulsion is a water-in-oil emulsion. In some embodiments, the nanoemulsion comprises squalene, sorbitan trioleate, polysorbate 80, DOTAP, or any combination thereof. In some embodiments, the expression vector is a plasmid vector, a bacterial vector or a viral expression vector. In some embodiments, the expression vector is a viral expression vector. In some embodiments, the viral expression vector is a DNA virus or an RNA virus. In some embodiments, the viral expression vector is replication defective, replication deficient, replication attenuated or replication competent. In some embodiments, the viral expression vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus. In some embodiments, the viral expression vector is from a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus). In some embodiments, the viral expression vector is an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV). In some embodiments, the viral expression vector is an arenavirus vector selected from Lymphocytic choriomeningitis mammarenavirus (LCMV) or Cali mammarenavirus (a.k.a. Pichinde mammarenavirus or Pichinde arenavirus). In some embodiments, the arenavirus vector comprises a bi-segmented genome. In some embodiments, the arenavirus vector comprises a tri-segmented genome. In some embodiments, the viral expression vector is a human adenovirus or a simian adenovirus. In some embodiments, the simian adenovirus is selected from a chimpanzee adenovirus, a gorilla adenovirus, and a rhesus adenovirus. In some embodiments, the viral expression vector is an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (ChAd), gorilla adenovirus, and rhesus adenovirus. In some embodiments, the ChAd is selected from ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), and ChAd147 (AdC147). In some embodiments, the ChAd is ChAd68. In some embodiments, the gorilla adenovirus is selected from GC44, GC45, and GC46. In some embodiments, the rhesus adenovirus (RhAd) is selected from RhAd51, RhAd52, RhAd53, RhAd54, RhAd55, RhAd56, RhAd57, RhAd58, RhAd59, RhAd60, RhAd61, RhAd62, RhAd63, RhAd64, RhAd65 and RhAd66. In some embodiments, the human adenovirus or simian adenovirus comprises a modification of one or more adenoviral genes. In some embodiments, the one or more adenoviral genes are selected from E1, E3, and E4ORF2-4. In some embodiments, the modification comprises a deletion, substitution, or insertion of one or more nucleotides into the one or more adenoviral genes. In some embodiments, the human adenovirus or simian adenovirus comprises a deletion of at least a portion of the E1 region. In some embodiments, the polynucleotide is inserted into the deletion of at least a portion of the E1 region. In some embodiments, the polynucleotide is operably linked to and under the control of a constitutive promoter. In some embodiments, the promoter is selected from a CMV promoter, a CAG promoter, an EF1a promoter, and a 26S sub-genomic promoter. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529. In some embodiments, the promoter sequence comprises or consists of a polynucleotide sequence of SEQ ID NO: 530. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 530. In some embodiments, the promoter sequence comprises or consists of a codon-optimized nucleic acid sequence of the nucleic acid sequence of SEQ ID NO: 530. In some embodiments, any of the compositions disclosed herein further comprise a pharmaceutically acceptable diluent, carrier or excipient. In some embodiments, any of the compositions or kits disclosed herein further comprise one or more of an adjuvant, an immunostimulator, a detergent, a micelle-forming agent, and an oil. In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein. In some embodiments, the cytokine selected from IL-2, IL-7, IL-12, IL-15, IL-18, IL-21, IFN-α, IFN-γ, GM-CSF, FLT3LG, and combinations and functional variants thereof. In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102). In some embodiments, the composition is formulated for administration via a route selected from the group consisting of intravenous, intramuscular, intradermal, subcutaneous and mucosal (e.g. buccal, intranasal, intrarectal, intravaginal). In some embodiments, the composition is formulated as a liquid. In some embodiments, the composition is lyophilized. In some embodiments, any of the compositions or kits disclosed herein further comprise one or more unitary doses of one or more additional therapeutic agents. In some embodiments, any of the compositions or kits disclosed herein further comprise GS 9620 (vesatolimod). In some embodiments, any of the compositions or kits disclosed herein further comprise lefitolimod (MGN-1703). In some embodiments, any of the compositions or kits disclosed herein further comprise a bnAb. In some embodiments, the bnAb is an HIV bnAb. In some embodiments, any of the compositions or kits disclosed herein further comprise (a) any of the SAMs or viral expression vectors disclosed herein; or (b) any of the LNPs, PNPs, nanoemulsions, or viral vectors comprising such SAMs or viral expression vectors disclosed herein. In some embodiments, the viral expression vector comprises a polynucleotide comprising (a) the nucleic sequence of any one of SEQ ID NOs: 520-521; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 520-521; or (c) a codon-optimized nucleic acid sequence of the nucleic sequence of any one of SEQ ID NOs: 520-521. In some embodiments, the SAM comprises a polynucleotide comprising (a) the nucleic sequence of any one of SEQ ID NOs: 522-523; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 522-523; or (c) a codon-optimized nucleic acid sequence of the nucleic sequence of any one of SEQ ID NOs: 522-523. In some embodiments, SAM or viral expression vector comprises a polynucleotide comprising (a) the nucleic sequence of any one of SEQ ID NOs: 524-526; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 524-526; or (c) a codon-optimized nucleic acid sequence of the nucleic sequence of any one of SEQ ID NOs: 524-526. In some embodiments, SAM or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 527-528, wherein percent identity is across the full length of the nucleic acid sequence of the polypeptide encoding any one of the HIV immunogen amino acid sequence of SEQ ID NOs: 527-528; or (c) a codon-optimized nucleic acid sequence of the nucleic sequence encoding the polypeptide of any one of SEQ ID NOs: 1-344. In some embodiments, SAM or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 1-344, wherein percent identity is across the full length of the nucleic acid sequence of the polypeptide encoding any one of the HIV immunogen amino acid sequence of SEQ ID NOs: 1-344; or (c) a codon-optimized nucleic acid sequence of the nucleic sequence encoding the polypeptide of any one of SEQ ID NOs: 1-344. In some embodiments, SAM or viral expression vector comprises a polynucleotide comprising (a) a nucleic sequence encoding the polypeptide of any one of SEQ ID NOs: 345-371, 373-377, 407-411, 422-423, and 430-435; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435; or (c) a codon-optimized nucleic acid sequence of the nucleic sequence encoding the polypeptide of any one of SEQ ID NOs: 345-371, 373-377, 407-411, 422-423, and 430-435.

Further disclosed herein are compositions or kits comprising (a) a polynucleotide comprising an HIV immunogen nucleic acid sequence that encodes any one of the HIV immunogen amino acid sequences of SEQ ID NOs: 527-528; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. Further disclosed herein are compositions or kits comprising (a) a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOS: 527-528, wherein percent identity is across the full length of the nucleic acid sequence of the polypeptide encoding any one of the HIV immunogen amino acid sequence of SEQ ID NOs: 527-528; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. Further disclosed herein are compositions or kits comprising (a) a polynucleotide comprising a codon-optimized nucleic acid sequence of an HIV immunogen nucleic acid sequence that encodes any one of the HIV immunogen amino acid sequences of SEQ ID NOs: 527-528; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. In some embodiments, any of the compositions disclosed herein further comprise a pharmaceutically acceptable diluent, carrier or excipient. In some embodiments, any of the compositions or kits disclosed herein further comprise one or more of an adjuvant, an immunostimulator, a detergent, a micelle-forming agent, and an oil. In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein. In some embodiments, the cytokine selected from IL-2, IL-7, IL-12, IL-15, IL-18, IL-21, IFN-α, IFN-γ, GM-CSF, FLT3LG, and combinations and functional variants thereof. In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102). In some embodiments, the composition is formulated for administration via a route selected from the group consisting of intravenous, intramuscular, intradermal, subcutaneous and mucosal (e.g. buccal, intranasal, intrarectal, intravaginal). In some embodiments, the composition is formulated as a liquid. In some embodiments, the composition is lyophilized. In some embodiments, any of the compositions or kits disclosed herein further comprise one or more unitary doses of one or more additional therapeutic agents. In some embodiments, any of the compositions or kits disclosed herein further comprise GS 9620 (vesatolimod). In some embodiments, any of the compositions or kits disclosed herein further comprise lefitolimod (MGN-1703). In some embodiments, any of the compositions or kits disclosed herein further comprise a bnAb. In some embodiments, the bnAb is an HIV bnAb. In some embodiments, any of the compositions or kits disclosed herein further comprise (a) any of the SAMs or viral expression vectors disclosed herein; or (b) any of the LNPs, PNPs, nanoemulsions, or viral vectors comprising such SAMs or viral expression vectors disclosed herein. In some embodiments, the viral expression vector comprises a polynucleotide comprising (a) the nucleic sequence of any one of SEQ ID NOs: 520-521; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 520-521; or (c) a codon-optimized nucleic acid sequence of the nucleic sequence of any one of SEQ ID NOs: 520-521. In some embodiments, the SAM comprises a polynucleotide comprising (a) the nucleic sequence of any one of SEQ ID NOs: 522-523; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 522-523; or (c) a codon-optimized nucleic acid sequence of the nucleic sequence of any one of SEQ ID NOs: 522-523. In some embodiments, SAM or viral expression vector comprises a polynucleotide comprising (a) the nucleic sequence of any one of SEQ ID NOs: 524-526; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 524-526; or (c) a codon-optimized nucleic acid sequence of the nucleic sequence of any one of SEQ ID NOs: 524-526. In some embodiments, SAM or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 527-528, wherein percent identity is across the full length of the nucleic acid sequence of the polypeptide encoding any one of the HIV immunogen amino acid sequence of SEQ ID NOs: 527-528; or (c) a codon-optimized nucleic acid sequence of the nucleic sequence encoding the polypeptide of any one of SEQ ID NOs: 1-344. In some embodiments, SAM or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 1-344, wherein percent identity is across the full length of the nucleic acid sequence of the polypeptide encoding any one of the HIV immunogen amino acid sequence of SEQ ID NOs: 1-344; or (c) a codon-optimized nucleic acid sequence of the nucleic sequence encoding the polypeptide of any one of SEQ ID NOs: 1-344. In some embodiments, SAM or viral expression vector comprises a polynucleotide comprising (a) a nucleic sequence encoding the polypeptide of any one of SEQ ID NOs: 345-371, 373-377, 407-411, 422-423, and 430-435; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435; or (c) a codon-optimized nucleic acid sequence of the nucleic sequence encoding the polypeptide of any one of SEQ ID NOs: 345-371, 373-377, 407-411, 422-423, and 430-435.

Further disclosed herein are compositions or kits comprising a modified viral genome. In some embodiments, the modified viral genome comprises a deletion of one or more genes encoding one or more viral structural proteins. In some embodiments, the modified viral genome is produced by replacing one or more viral structural proteins with any of the polynucleotides disclosed herein. In some embodiments, the modified viral genome is produced by replacing one or more viral structural proteins with a polynucleotide that encodes any of the polypeptides of SEQ ID NOs: 345-371, 373-377, 407-411, 422-423, 430-435, 527, and 528. In some embodiments, the modified viral genome is produced by replacing one or more viral structural proteins with a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 345-371, 373-377, 407-411, 422-423, 430-435, 527, and 528, wherein percent identity is across the full length of the polypeptide of SEQ ID NOs: 345-371, 373-377, 407-411, 422-423, 430-435, 527, and 528. In some embodiments, the modified viral genome is produced by replacing one or more viral structural proteins with a polynucleotide comprising a codon-optimized nucleic acid sequence that encodes any of the polypeptides of SEQ ID NOs: 345-371, 373-377, 407-411, 422-423, 430-435, 527, and 528. In some embodiments, the modified viral genome is produced by replacing one or more viral structural proteins with a polynucleotide encoding any of the polypeptides of SEQ ID NOs: 1-344. In some embodiments, the modified viral genome is produced by replacing one or more viral structural proteins with a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to a nucleic acid sequence that encodes any of the polypeptides of SEQ ID NOs: 1-344, wherein percent identity is across the full length of the polypeptide of 1-344. In some embodiments, the modified viral genome is produced by replacing one or more viral structural proteins with a polynucleotide comprising a codon-optimized nucleic acid sequence that encodes any one of the polypeptides of SEQ ID NOs: 1-344. In some embodiments, any of the compositions disclosed herein further comprise a pharmaceutically acceptable diluent, carrier or excipient. In some embodiments, any of the compositions or kits disclosed herein further comprise one or more of an adjuvant, an immunostimulator, a detergent, a micelle-forming agent, and an oil. In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein. In some embodiments, the cytokine selected from IL-2, IL-7, IL-12, IL-15, IL-18, IL-21, IFN-α, IFN-γ, GM-CSF, FLT3LG, and combinations and functional variants thereof. In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102). In some embodiments, the composition is formulated for administration via a route selected from the group consisting of intravenous, intramuscular, intradermal, subcutaneous and mucosal (e.g. buccal, intranasal, intrarectal, intravaginal). In some embodiments, the composition is formulated as a liquid. In some embodiments, the composition is lyophilized. In some embodiments, any of the compositions or kits disclosed herein further comprise one or more unitary doses of one or more additional therapeutic agents. In some embodiments, any of the compositions or kits disclosed herein further comprise GS 9620 (vesatolimod). In some embodiments, any of the compositions or kits disclosed herein further comprise lefitolimod (MGN-1703). In some embodiments, any of the compositions or kits disclosed herein further comprise a bnAb. In some embodiments, the bnAb is an HIV bnAb. In some embodiments, any of the compositions or kits disclosed herein further comprise (a) any of the SAMs or viral expression vectors disclosed herein; or (b) any of the LNPs, PNPs, nanoemulsions, or viral vectors comprising such SAMs or viral expression vectors disclosed herein. In some embodiments, the viral expression vector comprises a polynucleotide comprising (a) the nucleic sequence of any one of SEQ ID NOs: 520-521; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 520-521; or (c) a codon-optimized nucleic acid sequence of the nucleic sequence of any one of SEQ ID NOs: 520-521. In some embodiments, the SAM comprises a polynucleotide comprising (a) the nucleic sequence of any one of SEQ ID NOs: 522-523; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 522-523; or (c) a codon-optimized nucleic acid sequence of the nucleic sequence of any one of SEQ ID NOs: 522-523. In some embodiments, SAM or viral expression vector comprises a polynucleotide comprising (a) the nucleic sequence of any one of SEQ ID NOs: 524-526; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 524-526; or (c) a codon-optimized nucleic acid sequence of the nucleic sequence of any one of SEQ ID NOs: 524-526. In some embodiments, SAM or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 527-528, wherein percent identity is across the full length of the nucleic acid sequence of the polypeptide encoding any one of the HIV immunogen amino acid sequence of SEQ ID NOs: 527-528; or (c) a codon-optimized nucleic acid sequence of the nucleic sequence encoding the polypeptide of any one of SEQ ID NOs: 1-344. In some embodiments, SAM or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 1-344, wherein percent identity is across the full length of the nucleic acid sequence of the polypeptide encoding any one of the HIV immunogen amino acid sequence of SEQ ID NOs: 1-344; or (c) a codon-optimized nucleic acid sequence of the nucleic sequence encoding the polypeptide of any one of SEQ ID NOs: 1-344. In some embodiments, SAM or viral expression vector comprises a polynucleotide comprising (a) a nucleic sequence encoding the polypeptide of any one of SEQ ID NOs: 345-371, 373-377, 407-411, 422-423, and 430-435; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435; or (c) a codon-optimized nucleic acid sequence of the nucleic sequence encoding the polypeptide of any one of SEQ ID NOs: 345-371, 373-377, 407-411, 422-423, and 430-435.

Further disclosed herein are compositions or kits comprising (a) a polynucleotide comprising an HIV immunogen nucleic acid sequence that encodes any one of the HIV immunogen amino acid sequences of SEQ ID NOs: 1-344, wherein the polynucleotide is operably linked to one or more regulatory sequences; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. Further disclosed herein are compositions or kits comprising (a) a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 1-344, wherein percent identity is across the full length of the nucleic acid sequence of the polypeptide encoding any one of the HIV immunogen amino acid sequence of SEQ ID NOs: 1-344, and wherein the polynucleotide is operably linked to one or more regulatory sequences; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. Further disclosed herein are compositions or kits comprising (a) a polynucleotide comprising a codon-optimized nucleic acid sequence of an HIV immunogen nucleic acid sequence that encodes any one of the HIV immunogen amino acid sequences of SEQ ID NOs: 1-344, wherein the polynucleotide is operably linked to one or more regulatory sequences; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. In some embodiments, any of the compositions disclosed herein further comprise a pharmaceutically acceptable diluent, carrier or excipient. In some embodiments, any of the compositions or kits disclosed herein further comprise one or more of an adjuvant, an immunostimulator, a detergent, a micelle-forming agent, and an oil. In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein. In some embodiments, the cytokine selected from IL-2, IL-7, IL-12, IL-15, IL-18, IL-21, IFN-α, IFN-γ, GM-CSF, FLT3LG, and combinations and functional variants thereof. In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102). In some embodiments, the composition is formulated for administration via a route selected from the group consisting of intravenous, intramuscular, intradermal, subcutaneous and mucosal (e.g. buccal, intranasal, intrarectal, intravaginal). In some embodiments, the composition is formulated as a liquid. In some embodiments, the composition is lyophilized. In some embodiments, any of the compositions or kits disclosed herein further comprise one or more unitary doses of one or more additional therapeutic agents. In some embodiments, any of the compositions or kits disclosed herein further comprise GS 9620 (vesatolimod). In some embodiments, any of the compositions or kits disclosed herein further comprise lefitolimod (MGN-1703). In some embodiments, any of the compositions or kits disclosed herein further comprise a bnAb. In some embodiments, the bnAb is an HIV bnAb. In some embodiments, any of the compositions or kits disclosed herein further comprise (a) any of the SAMs or viral expression vectors disclosed herein; or (b) any of the LNPs, PNPs, nanoemulsions, or viral vectors comprising such SAMs or viral expression vectors disclosed herein. In some embodiments, the viral expression vector comprises a polynucleotide comprising (a) the nucleic sequence of any one of SEQ ID NOs: 520-521; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 520-521; or (c) a codon-optimized nucleic acid sequence of the nucleic sequence of any one of SEQ ID NOs: 520-521. In some embodiments, the SAM comprises a polynucleotide comprising (a) the nucleic sequence of any one of SEQ ID NOs: 522-523; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 522-523; or (c) a codon-optimized nucleic acid sequence of the nucleic sequence of any one of SEQ ID NOs: 522-523. In some embodiments, SAM or viral expression vector comprises a polynucleotide comprising (a) the nucleic sequence of any one of SEQ ID NOs: 524-526; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 524-526; or (c) a codon-optimized nucleic acid sequence of the nucleic sequence of any one of SEQ ID NOs: 524-526. In some embodiments, SAM or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 527-528, wherein percent identity is across the full length of the nucleic acid sequence of the polypeptide encoding any one of the HIV immunogen amino acid sequence of SEQ ID NOs: 527-528; or (c) a codon-optimized nucleic acid sequence of the nucleic sequence encoding the polypeptide of any one of SEQ ID NOs: 1-344. In some embodiments, SAM or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 1-344, wherein percent identity is across the full length of the nucleic acid sequence of the polypeptide encoding any one of the HIV immunogen amino acid sequence of SEQ ID NOs: 1-344; or (c) a codon-optimized nucleic acid sequence of the nucleic sequence encoding the polypeptide of any one of SEQ ID NOs: 1-344. In some embodiments, SAM or viral expression vector comprises a polynucleotide comprising (a) a nucleic sequence encoding the polypeptide of any one of SEQ ID NOs: 345-371, 373-377, 407-411, 422-423, and 430-435; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435; or (c) a codon-optimized nucleic acid sequence of the nucleic sequence encoding the polypeptide of any one of SEQ ID NOs: 345-371, 373-377, 407-411, 422-423, and 430-435.

Further disclosed herein are compositions or kits comprising (a) a polynucleotide comprising an HIV immunogen nucleic acid sequence that encodes any one of the HIV immunogen amino acid sequences of SEQ ID NOs: 345-371, 373-377, 407-411, 422-423, and 430-435, wherein the polynucleotide is operably linked to one or more regulatory sequences; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. Further disclosed herein are compositions or kits comprising (a) a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435, and wherein the polynucleotide is operably linked to one or more regulatory sequences; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. Further disclosed herein are compositions or kits comprising (a) a polynucleotide comprising a codon-optimized nucleic acid sequence of an HIV immunogen nucleic acid sequence that encodes any one of the HIV immunogen amino acid sequences of SEQ ID NOs: 345-371, 373-377, 407-411, 422-423, and 430-435, wherein the polynucleotide is operably linked to one or more regulatory sequences; (b) a SAM, expression cassette, expression vector, or viral expression vector comprising such polynucleotide; or (c) a lipid nanoparticle (LNP), polymeric nanoparticles (PNP), nanoemulsions, or viral vector comprising such polynucleotide, SAM, expression cassette, expression vector, or viral expression vector. In some embodiments, any of the compositions disclosed herein further comprise a pharmaceutically acceptable diluent, carrier or excipient. In some embodiments, any of the compositions or kits disclosed herein further comprise one or more of an adjuvant, an immunostimulator, a detergent, a micelle-forming agent, and an oil. In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein. In some embodiments, the cytokine selected from IL-2, IL-7, IL-12, IL-15, IL-18, IL-21, IFN-α, IFN-γ, GM-CSF, FLT3LG, and combinations and functional variants thereof. In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102). In some embodiments, the composition is formulated for administration via a route selected from the group consisting of intravenous, intramuscular, intradermal, subcutaneous and mucosal (e.g. buccal, intranasal, intrarectal, intravaginal). In some embodiments, the composition is formulated as a liquid. In some embodiments, the composition is lyophilized. In some embodiments, any of the compositions or kits disclosed herein further comprise one or more unitary doses of one or more additional therapeutic agents. In some embodiments, any of the compositions or kits disclosed herein further comprise GS 9620 (vesatolimod). In some embodiments, any of the compositions or kits disclosed herein further comprise lefitolimod (MGN-1703). In some embodiments, any of the compositions or kits disclosed herein further comprise a bnAb. In some embodiments, the bnAb is an HIV bnAb. In some embodiments, any of the compositions or kits disclosed herein further comprise (a) any of the SAMs or viral expression vectors disclosed herein; or (b) any of the LNPs, PNPs, nanoemulsions, or viral vectors comprising such SAMs or viral expression vectors disclosed herein. In some embodiments, the viral expression vector comprises a polynucleotide comprising (a) the nucleic sequence of any one of SEQ ID NOs: 520-521; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 520-521; or (c) a codon-optimized nucleic acid sequence of the nucleic sequence of any one of SEQ ID NOs: 520-521. In some embodiments, the SAM comprises a polynucleotide comprising (a) the nucleic sequence of any one of SEQ ID NOs: 522-523; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 522-523; or (c) a codon-optimized nucleic acid sequence of the nucleic sequence of any one of SEQ ID NOs: 522-523. In some embodiments, SAM or viral expression vector comprises a polynucleotide comprising (a) the nucleic sequence of any one of SEQ ID NOs: 524-526; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 524-526; or (c) a codon-optimized nucleic acid sequence of the nucleic sequence of any one of SEQ ID NOs: 524-526. In some embodiments, SAM or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 527-528, wherein percent identity is across the full length of the nucleic acid sequence of the polypeptide encoding any one of the HIV immunogen amino acid sequence of SEQ ID NOs: 527-528; or (c) a codon-optimized nucleic acid sequence of the nucleic sequence encoding the polypeptide of any one of SEQ ID NOs: 1-344. In some embodiments, SAM or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 1-344, wherein percent identity is across the full length of the nucleic acid sequence of the polypeptide encoding any one of the HIV immunogen amino acid sequence of SEQ ID NOs: 1-344; or (c) a codon-optimized nucleic acid sequence of the nucleic sequence encoding the polypeptide of any one of SEQ ID NOs: 1-344. In some embodiments, SAM or viral expression vector comprises a polynucleotide comprising (a) a nucleic sequence encoding the polypeptide of any one of SEQ ID NOs: 345-371, 373-377, 407-411, 422-423, and 430-435; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435; or (c) a codon-optimized nucleic acid sequence of the nucleic sequence encoding the polypeptide of any one of SEQ ID NOs: 345-371, 373-377, 407-411, 422-423, and 430-435.

Further disclosed herein are compositions or kits comprising a self-amplifying RNA (SAM) comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOS: 345-371, 373-377, 407-411, 422-423, 430-435, 527, and 528, wherein percent identity is across the full length of SEQ ID NOs: 345-371, 373-377, 407-411, 422-423, 430-435, 527, and 528; (e) a fourth polynucleotide sequence comprising nucleotides 10667 to 10952 of SEQ ID NO: 522; and (f) a polyA region. In some embodiments, the third polynucleotide is operably linked to and under the control of the promoter sequence. In some embodiments, the promoter sequence is a constitutive promoter. In some embodiments, the promoter is selected from a CMV promoter, a CAG promoter, an EF1a promoter, and a 26S sub-genomic promoter. In some embodiments, the promoter comprises or consists of any of the promoter sequences of SEQ ID NOs: 529-530. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529. In some embodiments, any of the compositions disclosed herein further comprise a pharmaceutically acceptable diluent, carrier or excipient. In some embodiments, any of the compositions or kits disclosed herein further comprise one or more of an adjuvant, an immunostimulator, a detergent, a micelle-forming agent, and an oil. In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein. In some embodiments, the cytokine selected from IL-2, IL-7, IL-12, IL-15, IL-18, IL-21, IFN-α, IFN-γ, GM-CSF, FLT3LG, and combinations and functional variants thereof. In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102). In some embodiments, the composition is formulated for administration via a route selected from the group consisting of intravenous, intramuscular, intradermal, subcutaneous and mucosal (e.g. buccal, intranasal, intrarectal, intravaginal). In some embodiments, the composition is formulated as a liquid. In some embodiments, the composition is lyophilized. In some embodiments, any of the compositions or kits disclosed herein further comprise one or more unitary doses of one or more additional therapeutic agents. In some embodiments, any of the compositions or kits disclosed herein further comprise GS 9620 (vesatolimod). In some embodiments, any of the compositions or kits disclosed herein further comprise lefitolimod (MGN-1703). In some embodiments, any of the compositions or kits disclosed herein further comprise a bnAb. In some embodiments, the bnAb is an HIV bnAb. In some embodiments, any of the compositions or kits disclosed herein further comprise any of the viral vector or viral expression vectors disclosed herein. In some embodiments, the viral vector or viral expression vector comprises a polynucleotide comprising (a) the nucleic sequence of any one of SEQ ID NOs: 520-521; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 520-521; or (c) a codon-optimized nucleic acid sequence of the nucleic sequence of any one of SEQ ID NOs: 520-521. In some embodiments, the viral vector or viral expression vector comprises a polynucleotide comprising (a) the nucleic sequence of any one of SEQ ID NOs: 524-526; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 524-526; or (c) a codon-optimized nucleic acid sequence of the nucleic sequence of any one of SEQ ID NOs: 524-526. In some embodiments, the viral vector or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 527-528, wherein percent identity is across the full length of the nucleic acid sequence of the polypeptide encoding any one of the HIV immunogen amino acid sequence of SEQ ID NOs: 527-528; or (c) a codon-optimized nucleic acid sequence of the nucleic sequence encoding the polypeptide of any one of SEQ ID NOs: 1-344. In some embodiments, the viral vector or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 1-344, wherein percent identity is across the full length of the nucleic acid sequence of the polypeptide encoding any one of the HIV immunogen amino acid sequence of SEQ ID NOs: 1-344; or (c) a codon-optimized nucleic acid sequence of the nucleic sequence encoding the polypeptide of any one of SEQ ID NOs: 1-344. In some embodiments, the viral vector or viral expression vector comprises a polynucleotide comprising (a) a nucleic sequence encoding the polypeptide of any one of SEQ ID NOs: 345-371, 373-377, 407-411, 422-423, and 430-435; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435; or (c) a codon-optimized nucleic acid sequence of the nucleic sequence encoding the polypeptide of any one of SEQ ID NOs: 345-371, 373-377, 407-411, 422-423, and 430-435.

Further disclosed herein are compositions or kits comprising a self-amplifying RNA (SAM) comprising: (a) a first polynucleotide sequence comprising nucleotides 1 to 7512 of SEQ ID NO: 522; (b) a promoter sequence; (c) a second polynucleotide sequence comprising nucleotides 7537 to 7570 of SEQ ID NO: 522; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 10667 to 10952 of SEQ ID NO: 522; and (f) a polyA region. In some embodiments, the third polynucleotide sequence encodes two or more polypeptide segments. In some embodiments, the third polynucleotide sequence encodes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344. In some embodiments, one or more of the polypeptide segments is abutted or fused to an adjacent polypeptide segment. In some embodiments, one or more of the polypeptide segments is joined to an adjacent polypeptide segment by one or more peptide linkers. In some embodiments, the one or more peptide linkers is selected from one or more of a polyalanine linker, a polyglycine linker, a cleavable linker, a flexible linker, a rigid linker, a Nef linking sequence, and combinations thereof. In some embodiments, the polyalanine linker comprises or consists of 2 or 3 contiguous alanine residues, e.g. AA, AAA, AAY or AAX, wherein X is any amino acid (e.g. A, C, D, E, F, G, H, I, K, L, M, N, P, Q, R, S, T, V, Y). In some embodiments, the flexible linker or polyglycine linker comprises or consists of GG, GGS, GSG or GGGS (SEQ ID NO: 421). In some embodiments, the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof. In some embodiments, the cleavable linker comprises or consists of a furin recognition/cleavage site selected from the group consisting of RAKR (SEQ ID NO: 381), REKR (SEQ ID NO: 382) and RRKR (SEQ ID NO: 383). In some embodiments, the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388). In some embodiments, the cleavable linker comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388). In some embodiments, the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), wherein percent identity is across the full length of SEQ ID NOs: 389-391. In some embodiments, the Nef linking sequence comprises or consists of an amino acid sequence selected from VHAGPIA (SEQ ID NO: 389), VHAGPVA (SEQ ID NO: 390) and GALDI (SEQ ID NO: 391). In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529. In some embodiments, any of the compositions disclosed herein further comprise a pharmaceutically acceptable diluent, carrier or excipient. In some embodiments, any of the compositions or kits disclosed herein further comprise one or more of an adjuvant, an immunostimulator, a detergent, a micelle-forming agent, and an oil. In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein. In some embodiments, the cytokine selected from IL-2, IL-7, IL-12, IL-15, IL-18, IL-21, IFN-α, IFN-γ, GM-CSF, FLT3LG, and combinations and functional variants thereof. In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102). In some embodiments, the composition is formulated for administration via a route selected from the group consisting of intravenous, intramuscular, intradermal, subcutaneous and mucosal (e.g. buccal, intranasal, intrarectal, intravaginal). In some embodiments, the composition is formulated as a liquid. In some embodiments, the composition is lyophilized. In some embodiments, any of the compositions or kits disclosed herein further comprise one or more unitary doses of one or more additional therapeutic agents. In some embodiments, any of the compositions or kits disclosed herein further comprise GS 9620 (vesatolimod). In some embodiments, any of the compositions or kits disclosed herein further comprise lefitolimod (MGN-1703). In some embodiments, any of the compositions or kits disclosed herein further comprise a bnAb. In some embodiments, the bnAb is an HIV bnAb. In some embodiments, any of the compositions or kits disclosed herein further comprise any of the viral vector or viral expression vectors disclosed herein. In some embodiments, the viral vector or viral expression vector comprises a polynucleotide comprising (a) the nucleic sequence of any one of SEQ ID NOs: 520-521; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 520-521; or (c) a codon-optimized nucleic acid sequence of the nucleic sequence of any one of SEQ ID NOs: 520-521. In some embodiments, the viral vector or viral expression vector comprises a polynucleotide comprising (a) the nucleic sequence of any one of SEQ ID NOs: 524-526; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 524-526; or (c) a codon-optimized nucleic acid sequence of the nucleic sequence of any one of SEQ ID NOs: 524-526. In some embodiments, the viral vector or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 527-528, wherein percent identity is across the full length of the nucleic acid sequence of the polypeptide encoding any one of the HIV immunogen amino acid sequence of SEQ ID NOs: 527-528; or (c) a codon-optimized nucleic acid sequence of the nucleic sequence encoding the polypeptide of any one of SEQ ID NOs: 1-344. In some embodiments, the viral vector or viral expression vector comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 1-344, wherein percent identity is across the full length of the nucleic acid sequence of the polypeptide encoding any one of the HIV immunogen amino acid sequence of SEQ ID NOs: 1-344; or (c) a codon-optimized nucleic acid sequence of the nucleic sequence encoding the polypeptide of any one of SEQ ID NOs: 1-344. In some embodiments, the viral vector or viral expression vector comprises a polynucleotide comprising (a) a nucleic sequence encoding the polypeptide of any one of SEQ ID NOs: 345-371, 373-377, 407-411, 422-423, and 430-435; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435; or (c) a codon-optimized nucleic acid sequence of the nucleic sequence encoding the polypeptide of any one of SEQ ID NOs: 345-371, 373-377, 407-411, 422-423, and 430-435.

Further disclosed herein are compositions or kits comprising (I) an expression vector comprising: (a) a first polynucleotide sequence comprising nucleotides 1 to 593 of SEQ ID NO: 520; (b) a promoter sequence; (b) a second polynucleotide sequence comprising nucleotides 1196 to 1255 of SEQ ID NO: 520; (c) a third polynucleotide sequence encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (d) a fourth polynucleotide sequence comprising nucleotides 4352 to 4395 of SEQ ID NO: 520; (e) a polyA sequence; and (f) a fifth polynucleotide sequence comprising nucleotides 4620 to 32350 of SEQ ID NO: 520; or (II) a viral vector comprising such expression vector. In some embodiments, any of the compositions disclosed herein further comprise a pharmaceutically acceptable diluent, carrier or excipient. In some embodiments, any of the compositions or kits disclosed herein further comprise one or more of an adjuvant, an immunostimulator, a detergent, a micelle-forming agent, and an oil. In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein. In some embodiments, the cytokine selected from IL-2, IL-7, IL-12, IL-15, IL-18, IL-21, IFN-α, IFN-γ, GM-CSF, FLT3LG, and combinations and functional variants thereof. In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102). In some embodiments, the composition is formulated for administration via a route selected from the group consisting of intravenous, intramuscular, intradermal, subcutaneous and mucosal (e.g. buccal, intranasal, intrarectal, intravaginal). In some embodiments, the composition is formulated as a liquid. In some embodiments, the composition is lyophilized. In some embodiments, any of the compositions or kits disclosed herein further comprise one or more unitary doses of one or more additional therapeutic agents. In some embodiments, any of the compositions or kits disclosed herein further comprise GS 9620 (vesatolimod). In some embodiments, any of the compositions or kits disclosed herein further comprise lefitolimod (MGN-1703). In some embodiments, any of the compositions or kits disclosed herein further comprise a bnAb. In some embodiments, the bnAb is an HIV bnAb. In some embodiments, any of the compositions or kits disclosed herein further comprise any of the SAMs disclosed herein, or a LNP, PNP, microemulsion or viral vector comprising such SAMs. In some embodiments, the SAM comprises a polynucleotide comprising (a) the nucleic sequence of any one of SEQ ID NOs: 522-523; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 522-523; or (c) a codon-optimized nucleic acid sequence of the nucleic sequence of any one of SEQ ID NOs: 522-523. In some embodiments, the SAM comprises a polynucleotide comprising (a) the nucleic sequence of any one of SEQ ID NOS: 524-526; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 524-526; or (c) a codon-optimized nucleic acid sequence of the nucleic sequence of any one of SEQ ID NOs: 524-526. In some embodiments, the SAM comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 527-528, wherein percent identity is across the full length of the nucleic acid sequence of the polypeptide encoding any one of the HIV immunogen amino acid sequence of SEQ ID NOs: 527-528; or (c) a codon-optimized nucleic acid sequence of the nucleic sequence encoding the polypeptide of any one of SEQ ID NOs: 1-344. In some embodiments, the SAM comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 1-344, wherein percent identity is across the full length of the nucleic acid sequence of the polypeptide encoding any one of the HIV immunogen amino acid sequence of SEQ ID NOs: 1-344; or (c) a codon-optimized nucleic acid sequence of the nucleic sequence encoding the polypeptide of any one of SEQ ID NOs: 1-344. In some embodiments, the SAM comprises a polynucleotide comprising (a) a nucleic sequence encoding the polypeptide of any one of SEQ ID NOs: 345-371, 373-377, 407-411, 422-423, and 430-435; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435; or (c) a codon-optimized nucleic acid sequence of the nucleic sequence encoding the polypeptide of any one of SEQ ID NOs: 345-371, 373-377, 407-411, 422-423, and 430-435.

Further disclosed herein are compositions or kits comprising (I) an expression vector comprising: (a) a first polynucleotide sequence comprising nucleotides 1 to 593 of SEQ ID NO: 520; (b) a promoter sequence; (c) a second polynucleotide sequence comprising nucleotides 1196 to 1255 of SEQ ID NO: 520; (d) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (e) a fourth polynucleotide sequence comprising nucleotides 4352 to 4395 of SEQ ID NO: 520; (f) a polyA sequence; and (g) a fifth polynucleotide sequence comprising nucleotides 4620 to 32350 of SEQ ID NO: 520; or (II) a viral vector comprising such expression vector. In some embodiments, the third polynucleotide sequence encodes two or more polypeptide segments. In some embodiments, the third polynucleotide sequence encodes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344. In some embodiments, one or more of the polypeptide segments is abutted or fused to an adjacent polypeptide segment. In some embodiments, one or more of the polypeptide segments is joined to an adjacent polypeptide segment by one or more peptide linkers. In some embodiments, the one or more peptide linkers is selected from one or more of a polyalanine linker, a polyglycine linker, a cleavable linker, a flexible linker, a rigid linker, a Nef linking sequence, and combinations thereof. In some embodiments, the polyalanine linker comprises or consists of 2 or 3 contiguous alanine residues, e.g. AA, AAA, AAY or AAX, wherein X is any amino acid (e.g. A, C, D, E, F, G, H, I, K, L, M, N, P, Q, R, S, T, V, Y). In some embodiments, the flexible linker or polyglycine linker comprises or consists of GG, GGS, GSG or GGGS (SEQ ID NO: 421). In some embodiments, the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof. In some embodiments, the cleavable linker comprises or consists of a furin recognition/cleavage site selected from the group consisting of RAKR (SEQ ID NO: 381), REKR (SEQ ID NO: 382) and RRKR (SEQ ID NO: 383). In some embodiments, the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388), wherein percent identity is across the full length of SEQ ID NOs: 384-388. In some embodiments, the cleavable linker comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388). In some embodiments, the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), wherein percent identity is across the full length of SEQ ID NOs: 389-391. In some embodiments, the Nef linking sequence comprises or consists of an amino acid sequence selected from VHAGPIA (SEQ ID NO: 389), VHAGPVA (SEQ ID NO: 390) and GALDI (SEQ ID NO: 391). In some embodiments, the promoter sequence is a constitutive promoter. In some embodiments, the promoter is selected from a CMV promoter, a CAG promoter, an EF1a promoter, and a 26S sub-genomic promoter. In some embodiments, the promoter sequence comprises or consists of a polynucleotide sequence of SEQ ID NO: 529. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 529. In some embodiments, the promoter sequence comprises or consists of a codon-optimized nucleic acid sequence of the nucleic acid sequence of SEQ ID NO: 529. In some embodiments, the promoter sequence comprises or consists of a polynucleotide sequence of SEQ ID NO: 530. In some embodiments, the promoter sequence comprises or consists of a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 530. In some embodiments, the promoter sequence comprises or consists of a codon-optimized nucleic acid sequence of the nucleic acid sequence of SEQ ID NO: 530. In some embodiments, any of the compositions disclosed herein further comprise a pharmaceutically acceptable diluent, carrier or excipient. In some embodiments, any of the compositions or kits disclosed herein further comprise one or more of an adjuvant, an immunostimulator, a detergent, a micelle-forming agent, and an oil. In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein. In some embodiments, the cytokine selected from IL-2, IL-7, IL-12, IL-15, IL-18, IL-21, IFN-α, IFN-γ, GM-CSF, FLT3LG, and combinations and functional variants thereof. In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102). In some embodiments, the composition is formulated for administration via a route selected from the group consisting of intravenous, intramuscular, intradermal, subcutaneous and mucosal (e.g. buccal, intranasal, intrarectal, intravaginal). In some embodiments, the composition is formulated as a liquid. In some embodiments, the composition is lyophilized. In some embodiments, any of the compositions or kits disclosed herein further comprise one or more unitary doses of one or more additional therapeutic agents. In some embodiments, any of the compositions or kits disclosed herein further comprise GS 9620 (vesatolimod). In some embodiments, any of the compositions or kits disclosed herein further comprise lefitolimod (MGN-1703). In some embodiments, any of the compositions or kits disclosed herein further comprise a bnAb. In some embodiments, the bnAb is an HIV bnAb. In some embodiments, any of the compositions or kits disclosed herein further comprise any of the SAMs disclosed herein, or a LNP, PNP, microemulsion or viral vector comprising such SAMs. In some embodiments, the SAM comprises a polynucleotide comprising (a) the nucleic sequence of any one of SEQ ID NOs: 522-523; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 522-523; or (c) a codon-optimized nucleic acid sequence of the nucleic sequence of any one of SEQ ID NOs: 522-523. In some embodiments, the SAM comprises a polynucleotide comprising (a) the nucleic sequence of any one of SEQ ID NOs: 524-526; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 524-526; or (c) a codon-optimized nucleic acid sequence of the nucleic sequence of any one of SEQ ID NOs: 524-526. In some embodiments, the SAM comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 527-528, wherein percent identity is across the full length of the nucleic acid sequence of the polypeptide encoding any one of the HIV immunogen amino acid sequence of SEQ ID NOs: 527-528; or (c) a codon-optimized nucleic acid sequence of the nucleic sequence encoding the polypeptide of any one of SEQ ID NOs: 1-344. In some embodiments, the SAM comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 1-344, wherein percent identity is across the full length of the nucleic acid sequence of the polypeptide encoding any one of the HIV immunogen amino acid sequence of SEQ ID NOs: 1-344; or (c) a codon-optimized nucleic acid sequence of the nucleic sequence encoding the polypeptide of any one of SEQ ID NOs: 1-344. In some embodiments, the SAM comprises a polynucleotide comprising (a) a nucleic sequence encoding the polypeptide of any one of SEQ ID NOs: 345-371, 373-377, 407-411, 422-423, and 430-435; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435; or (c) a codon-optimized nucleic acid sequence of the nucleic sequence encoding the polypeptide of any one of SEQ ID NOs: 345-371, 373-377, 407-411, 422-423, and 430-435.

Further disclosed herein are compositions or kits comprising (I) a viral expression vector comprising the nucleic acid sequence of any one of SEQ ID NOs: 520-521. Further disclosed herein are viral vectors comprising a viral expression vector comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of any one of SEQ ID NOs: 520-521. Further disclosed herein are viral vectors comprising a viral expression vector comprising a codon-optimized nucleic acid sequence of the nucleic acid sequence of any one of SEQ ID NOS: 520-521; or (II) a viral vector comprising such viral expression vector. In some embodiments, any of the compositions disclosed herein further comprise a pharmaceutically acceptable diluent, carrier or excipient. In some embodiments, any of the compositions or kits disclosed herein further comprise one or more of an adjuvant, an immunostimulator, a detergent, a micelle-forming agent, and an oil. In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein. In some embodiments, the cytokine selected from IL-2, IL-7, IL-12, IL-15, IL-18, IL-21, IFN-α, IFN-γ, GM-CSF, FLT3LG, and combinations and functional variants thereof. In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102). In some embodiments, the composition is formulated for administration via a route selected from the group consisting of intravenous, intramuscular, intradermal, subcutaneous and mucosal (e.g. buccal, intranasal, intrarectal, intravaginal). In some embodiments, the composition is formulated as a liquid. In some embodiments, the composition is lyophilized. In some embodiments, any of the compositions or kits disclosed herein further comprise one or more unitary doses of one or more additional therapeutic agents. In some embodiments, any of the compositions or kits disclosed herein further comprise GS 9620 (vesatolimod). In some embodiments, any of the compositions or kits disclosed herein further comprise lefitolimod (MGN-1703). In some embodiments, any of the compositions or kits disclosed herein further comprise a bnAb. In some embodiments, the bnAb is an HIV bnAb. In some embodiments, any of the compositions or kits disclosed herein further comprise any of the SAMs disclosed herein, or a LNP, PNP, microemulsion or viral vector comprising such SAMs. In some embodiments, the SAM comprises a polynucleotide comprising (a) the nucleic sequence of any one of SEQ ID NOs: 522-523; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 522-523; or (c) a codon-optimized nucleic acid sequence of the nucleic sequence of any one of SEQ ID NOs: 522-523. In some embodiments, the SAM comprises a polynucleotide comprising (a) the nucleic sequence of any one of SEQ ID NOs: 524-526; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence of any one of SEQ ID NOs: 524-526; or (c) a codon-optimized nucleic acid sequence of the nucleic sequence of any one of SEQ ID NOs: 524-526. In some embodiments, the SAM comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 527-528, wherein percent identity is across the full length of the nucleic acid sequence of the polypeptide encoding any one of the HIV immunogen amino acid sequence of SEQ ID NOs: 527-528; or (c) a codon-optimized nucleic acid sequence of the nucleic sequence encoding the polypeptide of any one of SEQ ID NOs: 1-344. In some embodiments, the SAM comprises a polynucleotide comprising (a) a nucleic sequence encoding the polypeptide of any one of SEQ ID NOs: 527-528; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 1-344, wherein percent identity is across the full length of the nucleic acid sequence of the polypeptide encoding any one of the HIV immunogen amino acid sequence of SEQ ID NOs: 1-344; or (c) a codon-optimized nucleic acid sequence of the nucleic sequence encoding the polypeptide of any one of SEQ ID NOs: 1-344. In some embodiments, the SAM comprises a polynucleotide comprising (a) a nucleic sequence encoding the polypeptide of any one of SEQ ID NOs: 345-371, 373-377, 407-411, 422-423, and 430-435; (b) a nucleic sequence is at least 80%, 85%, 86%, 87%, 88%, 89%, 90%, 91%, 92%, 93%, 94%, 95%, 96%, 97%, 98%, 99%, or 100% identical to the nucleic sequence encoding the polypeptide of any one of SEQ ID NOs: 345-371, 373-377, 407-411, 422-423, and 430-435, wherein percent identity is across the full length of the nucleic acid sequence of the polypeptide encoding any one of the HIV immunogen amino acid sequence of SEQ ID NOs: 345-371, 373-377, 407-411, 422-423, and 430-435; or (c) a codon-optimized nucleic acid sequence of the nucleic sequence encoding the polypeptide of any one of SEQ ID NOs: 345-371, 373-377, 407-411, 422-423, and 430-435.

Further disclosed herein are compositions or kits comprising polypeptides comprising any of the HIV immunogen amino acid sequences of SEQ ID NOs: 527-528. Further disclosed herein are polypeptides comprising an amino acid sequence that is at least 80%, 85%, 86%, 87%, 88%, 89%, 90%, 91%, 92%, 93%, 94%, 95%, 96%, 97%, 98%, 99%, or 100% identical any of the HIV immunogen amino acid sequences of SEQ ID NOs: 527-528, wherein percent identity is across the full length of the HIV immunogen amino acid sequence of SEQ ID NOs: 527-528. In some embodiments, the composition comprises two or more polypeptides disclosed herein. In some embodiments, any of the compositions disclosed herein further comprise a pharmaceutically acceptable diluent, carrier or excipient. In some embodiments, any of the compositions or kits disclosed herein further comprise one or more of an adjuvant, an immunostimulator, a detergent, a micelle-forming agent, and an oil. In some embodiments, the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein. In some embodiments, the cytokine selected from IL-2, IL-7, IL-12, IL-15, IL-18, IL-21, IFN-α, IFN-γ, GM-CSF, FLT3LG, and combinations and functional variants thereof. In some embodiments, the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102). In some embodiments, the composition is formulated for administration via a route selected from the group consisting of intravenous, intramuscular, intradermal, subcutaneous and mucosal (e.g. buccal, intranasal, intrarectal, intravaginal). In some embodiments, the composition is formulated as a liquid. In some embodiments, the composition is lyophilized. In some embodiments, any of the compositions or kits disclosed herein further comprise one or more unitary doses of one or more additional therapeutic agents. In some embodiments, any of the compositions or kits disclosed herein further comprise GS 9620 (vesatolimod). In some embodiments, any of the compositions or kits disclosed herein further comprise lefitolimod (MGN-1703). In some embodiments, any of the compositions or kits disclosed herein further comprise a bnAb. In some embodiments, the bnAb is an HIV bnAb.

Further disclosed herein are methods for eliciting an immune response to human immunodeficiency virus (HIV) in a subject in need thereof. In some embodiments, the method comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method for eliciting an immune response to human immunodeficiency virus (HIV) in a subject in need thereof comprises administering to the subject (a) one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOs: 524-526; (b) a SAM, expression cassette, expression vector, or viral expression vector comprising such polynucleotide; or (c) a LNP, PNP, nanoemulsion, or viral vector comprising such polynucleotide, SAM, expression cassette, expression vector, or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, RNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/I1-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods of treating or preventing human immunodeficiency virus (HIV) in a subject in need thereof. In some embodiments, the method of treating or preventing human immunodeficiency virus (HIV) in a subject in need thereof comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method comprises administering to the subject (a) one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOs: 524-526; (b) a SAM, expression cassette, expression vector, or viral expression vector comprising such polynucleotide; or (c) a LNP, PNP, nanoemulsion, or viral vector comprising such polynucleotide, SAM, expression cassette, expression vector, or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods for eliciting an immune response to human immunodeficiency virus (HIV) in a subject in need thereof. In some embodiments, the method comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method for eliciting an immune response to human immunodeficiency virus (HIV) in a subject in need thereof comprises administering to the subject (a) one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; (b) a SAM, expression cassette, expression vector, or viral expression vector comprising such polynucleotide; or (c) a LNP, PNP, nanoemulsion, or viral vector comprising such polynucleotide, SAM, expression cassette, expression vector, or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods of treating or preventing human immunodeficiency virus (HIV) in a subject in need thereof. In some embodiments, the method of treating or preventing human immunodeficiency virus (HIV) in a subject in need thereof comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method comprises administering to the subject (a) one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; (b) a SAM, expression cassette, expression vector, or viral expression vector comprising such polynucleotide; or (c) a LNP, PNP, nanoemulsion, or viral vector comprising such polynucleotide, SAM, expression cassette, expression vector, or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/I1-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods for eliciting an immune response to human immunodeficiency virus (HIV) in a subject in need thereof. In some embodiments, the method comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method for eliciting an immune response to human immunodeficiency virus (HIV) in a subject in need thereof comprises administering to the subject (a) one or more polynucleotides comprising (i) a nucleic acid sequence encoding the polypeptide of any one of SEQ ID NOs: 1-344; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 1-344, wherein percent identity is across the full length of the amino acid sequence of SEQ ID NOs: 1-344; or (iii) a codon-optimized nucleic acid sequence of the nucleic acid sequence encoding the polypeptide of any one of SEQ ID NOs: 1-344; (b) a SAM, expression cassette, expression vector, or viral expression vector comprising such polynucleotide; or (c) a LNP, PNP, nanoemulsion, or viral vector comprising such polynucleotide, SAM, expression cassette, expression vector, or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, RNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp 120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods of treating or preventing human immunodeficiency virus (HIV) in a subject in need thereof. In some embodiments, the method of treating or preventing human immunodeficiency virus (HIV) in a subject in need thereof comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method comprises administering to the subject (a) one or more polynucleotides comprising (i) a nucleic acid sequence encoding the polypeptide of any one of SEQ ID NOs: 1-344; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 1-344, wherein percent identity is across the full length of the amino acid sequence of SEQ ID NOs: 1-344; or (iii) a codon-optimized nucleic acid sequence of the nucleic acid sequence encoding the polypeptide of any one of SEQ ID NOs: 1-344; (b) a SAM, expression cassette, expression vector, or viral expression vector comprising such polynucleotide; or (c) a LNP, PNP, nanoemulsion, or viral vector comprising such polynucleotide, SAM, expression cassette, expression vector, or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods for eliciting an immune response to human immunodeficiency virus (HIV) in a subject in need thereof. In some embodiments, the method comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method for eliciting an immune response to human immunodeficiency virus (HIV) in a subject in need thereof comprises administering to the subject (a) one or more polynucleotides comprising (i) a nucleic acid sequence encoding the polypeptide of any one of SEQ ID NOs: 345-371, 373-377, 407-411, 422-423, and 430-435; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 345-371, 373-377, 407-411, 422-423, and 430-435, wherein percent identity is across the full length of the amino acid sequence of SEQ ID NOs: 345-371, 373-377, 407-411, 422-423, and 430-435; or (iii) a codon-optimized nucleic acid sequence of the nucleic acid sequence encoding the polypeptide of any one of SEQ ID NOs: 345-371, 373-377, 407-411, 422-423, and 430-435; (b) a SAM, expression cassette, expression vector, or viral expression vector comprising such polynucleotide; or (c) a LNP, PNP, nanoemulsion, or viral vector comprising such polynucleotide, SAM, expression cassette, expression vector, or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/I1-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods of treating or preventing human immunodeficiency virus (HIV) in a subject in need thereof. In some embodiments, the method of treating or preventing human immunodeficiency virus (HIV) in a subject in need thereof comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method comprises administering to the subject (a) one or more polynucleotides comprising (i) a nucleic acid sequence encoding the polypeptide of any one of SEQ ID NOs: 345-371, 373-377, 407-411, 422-423, and 430-435; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 345-371, 373-377, 407-411, 422-423, and 430-435, wherein percent identity is across the full length of the amino acid sequence of SEQ ID NOs: 345-371, 373-377, 407-411, 422-423, and 430-435; or (iii) a codon-optimized nucleic acid sequence of the nucleic acid sequence encoding the polypeptide of any one of SEQ ID NOs: 345-371, 373-377, 407-411, 422-423, and 430-435; (b) a SAM, expression cassette, expression vector, or viral expression vector comprising such polynucleotide; or (c) a LNP, PNP, nanoemulsion, or viral vector comprising such polynucleotide, SAM, expression cassette, expression vector, or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods for eliciting an immune response to human immunodeficiency virus (HIV) in a subject in need thereof. In some embodiments, the method comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method for eliciting an immune response to human immunodeficiency virus (HIV) in a subject in need thereof comprises administering to the subject (a) one or more expression vectors or viral vectors comprising (i) the nucleic acid sequence of any one of SEQ ID NOs: 520-521; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521; or (iii) a codon-optimized nucleic acid sequence of the nucleic acid sequence of any one of SEQ ID NOs: 520-521; or (b) a LNP, PNP, nanoemulsion, or viral vector comprising such expression vector or viral expression vector. In some embodiments, the method further comprises administering to the subject any of the SAMs disclosed herein, or a LNP, PNP, nanoemulsion, or viral vector comprising such SAM disclosed herein. In some embodiments, the method further comprises administering to the subject (a) one or more SAMs comprising (i) the nucleic acid sequence of any one of SEQ ID NOs: 522-523; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523; or (iii) a codon-optimized nucleic acid sequence of the nucleic acid sequence of any one of SEQ ID NOS: 522-523; or (b) a LNP, PNP, nanoemulsion, or viral vector comprising such SAM. In some embodiments, the method further comprises administering to the subject (a) one or more SAMs comprising one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOs: 524-526; or (b) a LNP, PNP, nanoemulsion, or viral vector comprising such SAM. In some embodiments, the method further comprises administering to the subject (a) one or more SAMs comprising one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; or (b) a LNP, PNP, nanoemulsion, or viral vector comprising such SAM. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods of treating or preventing human immunodeficiency virus (HIV) in a subject in need thereof. In some embodiments, the method of treating or preventing human immunodeficiency virus (HIV) in a subject in need thereof comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method comprises administering to the subject (a) one or more expression vectors or viral vectors comprising (i) the nucleic acid sequence of any one of SEQ ID NOs: 520-521; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521; or (iii) a codon-optimized nucleic acid sequence of the nucleic acid sequence of any one of SEQ ID NOS: 520-521; or (b) a LNP, PNP, nanoemulsion, or viral vector comprising such expression vector or viral expression vector. In some embodiments, the method further comprises administering to the subject any of the SAMs disclosed herein, or a LNP, PNP, nanoemulsion, or viral vector comprising such SAM disclosed herein. In some embodiments, the method further comprises administering to the subject (a) one or more SAMs comprising (i) the nucleic acid sequence of any one of SEQ ID NOs: 522-523; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523; or (iii) a codon-optimized nucleic acid sequence of the nucleic acid sequence of any one of SEQ ID NOs: 522-523; or (b) a LNP, PNP, nanoemulsion, or viral vector comprising such SAM. In some embodiments, the method further comprises administering to the subject (a) one or more SAMs comprising one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOs: 524-526; or (b) a LNP, PNP, nanoemulsion, or viral vector comprising such SAM. In some embodiments, the method further comprises administering to the subject (a) one or more SAMs comprising one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; or (b) a LNP, PNP, nanoemulsion, or viral vector comprising such SAM. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, RNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/I1-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods for eliciting an immune response to human immunodeficiency virus (HIV) in a subject in need thereof. In some embodiments, the method comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method for eliciting an immune response to human immunodeficiency virus (HIV) in a subject in need thereof comprises administering to the subject (a) one or more expression vectors or viral vectors comprising (i) a first polynucleotide sequence comprising nucleotides 1 to 593 of SEQ ID NO: 520; (ii) a promoter sequence; (iii) a second polynucleotide sequence comprising nucleotides 1196 to 1255 of SEQ ID NO: 520; (iv) a third polynucleotide sequence encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOS: 345-371, 373-377, 407-411, 422-423, 430-435, 527, and 528; (v) a fourth polynucleotide sequence comprising nucleotides 4352 to 4395 of SEQ ID NO: 520; (vi) a polyA sequence; and (vii) a fifth polynucleotide sequence comprising nucleotides 4620 to 32350 of SEQ ID NO: 520; or (b) a LNP, PNP, nanoemulsion, or viral vector comprising such expression vector or viral expression vector. In some embodiments, the method further comprises administering to the subject any of the SAMs disclosed herein, or a LNP, PNP, nanoemulsion, or viral vector comprising such SAM disclosed herein. In some embodiments, the method further comprises administering to the subject (a) one or more SAMs comprising (i) the nucleic acid sequence of any one of SEQ ID NOs: 522-523; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523; or (iii) a codon-optimized nucleic acid sequence of the nucleic acid sequence of any one of SEQ ID NOs: 522-523; or (b) a LNP, PNP, nanoemulsion, or viral vector comprising such SAM. In some embodiments, the method further comprises administering to the subject (a) one or more SAMs comprising one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOs: 524-526; or (b) a LNP, PNP, nanoemulsion, or viral vector comprising such SAM. In some embodiments, the method further comprises administering to the subject (a) one or more SAMs comprising one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; or (b) a LNP, PNP, nanoemulsion, or viral vector comprising such SAM. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/I1-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods of treating or preventing human immunodeficiency virus (HIV) in a subject in need thereof. In some embodiments, the method of treating or preventing human immunodeficiency virus (HIV) in a subject in need thereof comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method comprises administering to the subject (a) one or more expression vectors or viral vectors comprising (i) a first polynucleotide sequence comprising nucleotides 1 to 593 of SEQ ID NO: 520; (ii) a promoter sequence; (iii) a second polynucleotide sequence comprising nucleotides 1196 to 1255 of SEQ ID NO: 520; (iv) a third polynucleotide sequence encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (v) a fourth polynucleotide sequence comprising nucleotides 4352 to 4395 of SEQ ID NO: 520; (vi) a polyA sequence; and (vii) a fifth polynucleotide sequence comprising nucleotides 4620 to 32350 of SEQ ID NO: 520; or (b) a LNP, PNP, nanoemulsion, or viral vector comprising such expression vector or viral expression vector. In some embodiments, the method further comprises administering to the subject any of the SAMs disclosed herein, or a LNP, PNP, nanoemulsion, or viral vector comprising such SAM disclosed herein. In some embodiments, the method further comprises administering to the subject (a) one or more SAMs comprising (i) the nucleic acid sequence of any one of SEQ ID NOs: 522-523; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523; or (iii) a codon-optimized nucleic acid sequence of the nucleic acid sequence of any one of SEQ ID NOs: 522-523; or (b) a LNP, PNP, nanoemulsion, or viral vector comprising such SAM. In some embodiments, the method further comprises administering to the subject (a) one or more SAMs comprising one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOs: 524-526; or (b) a LNP, PNP, nanoemulsion, or viral vector comprising such SAM. In some embodiments, the method further comprises administering to the subject (a) one or more SAMs comprising one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; or (b) a LNP, PNP, nanoemulsion, or viral vector comprising such SAM. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp 120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods for eliciting an immune response to human immunodeficiency virus (HIV) in a subject in need thereof. In some embodiments, the method comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method for eliciting an immune response to human immunodeficiency virus (HIV) in a subject in need thereof comprises administering to the subject (a) one or more expression vectors or viral vectors comprising (i) a first polynucleotide sequence comprising nucleotides 1 to 593 of SEQ ID NO: 520; (ii) a promoter sequence; (iii) a second polynucleotide sequence comprising nucleotides 1196 to 1255 of SEQ ID NO: 520; (iv) a third polynucleotide sequence encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (v) a fourth polynucleotide sequence comprising nucleotides 4352 to 4395 of SEQ ID NO: 520; (vi) a polyA sequence; and (vii) a fifth polynucleotide sequence comprising nucleotides 4620 to 32350 of SEQ ID NO: 520; or (b) a LNP, PNP, nanoemulsion, or viral vector comprising such expression vector or viral expression vector. In some embodiments, the method further comprises administering to the subject any of the SAMs disclosed herein, or a LNP, PNP, nanoemulsion, or viral vector comprising such SAM disclosed herein. In some embodiments, the method further comprises administering to the subject (a) one or more SAMs comprising (i) the nucleic acid sequence of any one of SEQ ID NOs: 522-523; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523; or (iii) a codon-optimized nucleic acid sequence of the nucleic acid sequence of any one of SEQ ID NOs: 522-523; or (b) a LNP, PNP, nanoemulsion, or viral vector comprising such SAM. In some embodiments, the method further comprises administering to the subject (a) one or more SAMs comprising one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOs: 524-526; or (b) a LNP, PNP, nanoemulsion, or viral vector comprising such SAM. In some embodiments, the method further comprises administering to the subject (a) one or more SAMs comprising one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; or (b) a LNP, PNP, nanoemulsion, or viral vector comprising such SAM. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, RNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, RNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods of treating or preventing human immunodeficiency virus (HIV) in a subject in need thereof. In some embodiments, the method of treating or preventing human immunodeficiency virus (HIV) in a subject in need thereof comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method comprises administering to the subject (a) one or more expression vectors or viral vectors comprising (i) a first polynucleotide sequence comprising nucleotides 1 to 593 of SEQ ID NO: 520; (ii) a promoter sequence; (iii) a second polynucleotide sequence comprising nucleotides 1196 to 1255 of SEQ ID NO: 520; (iv) a third polynucleotide sequence encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (v) a fourth polynucleotide sequence comprising nucleotides 4352 to 4395 of SEQ ID NO: 520; (vi) a polyA sequence; and (vii) a fifth polynucleotide sequence comprising nucleotides 4620 to 32350 of SEQ ID NO: 520; or (b) a LNP, PNP, nanoemulsion, or viral vector comprising such expression vector or viral expression vector. In some embodiments, the method further comprises administering to the subject any of the SAMs disclosed herein, or a LNP, PNP, nanoemulsion, or viral vector comprising such SAM disclosed herein. In some embodiments, the method further comprises administering to the subject (a) one or more SAMs comprising (i) the nucleic acid sequence of any one of SEQ ID NOs: 522-523; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523; or (iii) a codon-optimized nucleic acid sequence of the nucleic acid sequence of any one of SEQ ID NOs: 522-523; or (b) a LNP, PNP, nanoemulsion, or viral vector comprising such SAM. In some embodiments, the method further comprises administering to the subject (a) one or more SAMs comprising one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOs: 524-526; or (b) a LNP, PNP, nanoemulsion, or viral vector comprising such SAM. In some embodiments, the method further comprises administering to the subject (a) one or more SAMs comprising one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; or (b) a LNP, PNP, nanoemulsion, or viral vector comprising such SAM. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods for eliciting an immune response to human immunodeficiency virus (HIV) in a subject in need thereof. In some embodiments, the method comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method for eliciting an immune response to human immunodeficiency virus (HIV) in a subject in need thereof comprises administering to the subject (a) one or more SAMs comprising (i) the nucleic acid sequence of any one of SEQ ID NOs: 522-523; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523; or (iii) a codon-optimized nucleic acid sequence of the nucleic acid sequence of any one of SEQ ID NOs: 522-523; or (b) a LNP, PNP, nanoemulsion, or viral vector comprising such SAM. In some embodiments, the method further comprises administering to the subject any of the expression vectors or viral expression vectors disclosed herein, or a LNP, PNP, nanoemulsion, or viral vector comprising such expression vector or viral expression vector disclosed herein. In some embodiments, the method further comprises administering to the subject (a) one or more expression vectors or viral expression vectors comprising (i) the nucleic acid sequence of any one of SEQ ID NOs: 520-521; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521; or (iii) a codon-optimized nucleic acid sequence of the nucleic acid sequence of any one of SEQ ID NOs: 520-521; or (b) a LNP, PNP, nanoemulsion, or viral vector comprising such expression vector or viral expression vector. In some embodiments, the method further comprises administering to the subject (a) one or more expression vectors or viral expression vectors comprising one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOS: 524-526; or (b) a LNP, PNP, nanoemulsion, or viral vector comprising such expression vector or viral expression vector. In some embodiments, the method further comprises administering to the subject (a) one or more expression vectors or viral expression vectors comprising one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; or (b) a LNP, PNP, nanoemulsion, or viral vector comprising such expression vector or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods of treating or preventing human immunodeficiency virus (HIV) in a subject in need thereof. In some embodiments, the method of treating or preventing human immunodeficiency virus (HIV) in a subject in need thereof comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method comprises administering to the subject (a) one or more SAMs comprising (i) the nucleic acid sequence of any one of SEQ ID NOs: 522-523; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523; or (iii) a codon-optimized nucleic acid sequence of the nucleic acid sequence of any one of SEQ ID NOs: 522-523; or (b) a LNP, PNP, nanoemulsion, or viral vector comprising such SAM. In some embodiments, the method further comprises administering to the subject any of the expression vectors or viral expression vectors disclosed herein, or a LNP, PNP, nanoemulsion, or viral vector comprising such expression vector or viral expression vector disclosed herein. In some embodiments, the method further comprises administering to the subject (a) one or more expression vectors or viral expression vectors comprising (i) the nucleic acid sequence of any one of SEQ ID NOs: 520-521; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521; or (iii) a codon-optimized nucleic acid sequence of the nucleic acid sequence of any one of SEQ ID NOs: 520-521; or (b) a LNP, PNP, nanoemulsion, or viral vector comprising such expression vector or viral expression vector. In some embodiments, the method further comprises administering to the subject (a) one or more expression vectors or viral expression vectors comprising one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOS: 524-526; or (b) a LNP, PNP, nanoemulsion, or viral vector comprising such expression vector or viral expression vector. In some embodiments, the method further comprises administering to the subject (a) one or more expression vectors or viral expression vectors comprising one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; or (b) a LNP, PNP, nanoemulsion, or viral vector comprising such expression vector or viral expression vector. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/I1-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods for eliciting an immune response to human immunodeficiency virus (HIV) in a subject in need thereof. In some embodiments, the method comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method for eliciting an immune response to human immunodeficiency virus (HIV) in a subject in need thereof comprises administering to the subject (a) one or more SAMs comprising (i) a first polynucleotide sequence comprising nucleotides 1 to 7512 of SEQ ID NO: 522; (ii) a promoter sequence; (iii) a second polynucleotide sequence comprising nucleotides 7537 to 7570 of SEQ ID NO: 522; (iv) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (v) a fourth polynucleotide sequence comprising nucleotides 10667 to 10952 of SEQ ID NO: 522; and (vi) a polyA region; or (b) a LNP, PNP, nanoemulsion, or viral vector comprising such SAM. In some embodiments, the method further comprises administering to the subject any of the expression vectors or viral expression vectors disclosed herein, or a LNP, PNP, nanoemulsion, or viral vector comprising such expression vector or viral expression vector disclosed herein. In some embodiments, the method further comprises administering to the subject (a) one or more expression vectors or viral expression vectors comprising (i) the nucleic acid sequence of any one of SEQ ID NOs: 520-521; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521; or (iii) a codon-optimized nucleic acid sequence of the nucleic acid sequence of any one of SEQ ID NOs: 520-521; or (b) a LNP, PNP, nanoemulsion, or viral vector comprising such expression vector or viral expression vector. In some embodiments, the method further comprises administering to the subject (a) one or more expression vectors or viral expression vectors comprising one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOS: 524-526; or (b) a LNP, PNP, nanoemulsion, or viral vector comprising such expression vector or viral expression vector. In some embodiments, the method further comprises administering to the subject (a) one or more expression vectors or viral expression vectors comprising one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; or (b) a LNP, PNP, nanoemulsion, or viral vector comprising such expression vector or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, RNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods of treating or preventing human immunodeficiency virus (HIV) in a subject in need thereof. In some embodiments, the method of treating or preventing human immunodeficiency virus (HIV) in a subject in need thereof comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method of treating or preventing human immunodeficiency virus (HIV) in a subject in need thereof comprises administering to the subject (a) one or more SAMs comprising (i) a first polynucleotide sequence comprising nucleotides 1 to 7512 of SEQ ID NO: 522; (ii) a promoter sequence; (iii) a second polynucleotide sequence comprising nucleotides 7537 to 7570 of SEQ ID NO: 522; (iv) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (v) a fourth polynucleotide sequence comprising nucleotides 10667 to 10952 of SEQ ID NO: 522; and (vi) a polyA region; or (b) a LNP, PNP, nanoemulsion, or viral vector comprising such SAM. In some embodiments, the method further comprises administering to the subject any of the expression vectors or viral expression vectors disclosed herein, or a LNP, PNP, nanoemulsion, or viral vector comprising such expression vector or viral expression vector disclosed herein. In some embodiments, the method further comprises administering to the subject (a) one or more expression vectors or viral expression vectors comprising (i) the nucleic acid sequence of any one of SEQ ID NOs: 520-521; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521; or (iii) a codon-optimized nucleic acid sequence of the nucleic acid sequence of any one of SEQ ID NOs: 520-521; or (b) a LNP, PNP, nanoemulsion, or viral vector comprising such expression vector or viral expression vector. In some embodiments, the method further comprises administering to the subject (a) one or more expression vectors or viral expression vectors comprising one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOs: 524-526; or (b) a LNP, PNP, nanoemulsion, or viral vector comprising such expression vector or viral expression vector. In some embodiments, the method further comprises administering to the subject (a) one or more expression vectors or viral expression vectors comprising one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; or (b) a LNP, PNP, nanoemulsion, or viral vector comprising such expression vector or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods for eliciting an immune response to human immunodeficiency virus (HIV) in a subject in need thereof. In some embodiments, the method comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method for eliciting an immune response to human immunodeficiency virus (HIV) in a subject in need thereof comprises administering to the subject (a) one or more SAMs comprising (i) a first polynucleotide sequence comprising nucleotides 1 to 7512 of SEQ ID NO: 522; (ii) a promoter sequence; (iii) a second polynucleotide sequence comprising nucleotides 7537 to 7570 of SEQ ID NO: 522; (iv) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (v) a fourth polynucleotide sequence comprising nucleotides 10667 to 10952 of SEQ ID NO: 522; and (vi) a polyA region; or (b) a LNP, PNP, nanoemulsion, or viral vector comprising such SAM. In some embodiments, the method further comprises administering to the subject any of the expression vectors or viral expression vectors disclosed herein, or a LNP, PNP, nanoemulsion, or viral vector comprising such expression vector or viral expression vector disclosed herein. In some embodiments, the method further comprises administering to the subject (a) one or more expression vectors or viral expression vectors comprising (i) the nucleic acid sequence of any one of SEQ ID NOs: 520-521; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521; or (iii) a codon-optimized nucleic acid sequence of the nucleic acid sequence of any one of SEQ ID NOs: 520-521; or (b) a LNP, PNP, nanoemulsion, or viral vector comprising such expression vector or viral expression vector. In some embodiments, the method further comprises administering to the subject (a) one or more expression vectors or viral expression vectors comprising one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOs: 524-526; or (b) a LNP, PNP, nanoemulsion, or viral vector comprising such expression vector or viral expression vector. In some embodiments, the method further comprises administering to the subject (a) one or more expression vectors or viral expression vectors comprising one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; or (b) a LNP, PNP, nanoemulsion, or viral vector comprising such expression vector or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g. buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, RNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

Further disclosed herein are methods of treating or preventing human immunodeficiency virus (HIV) in a subject in need thereof. In some embodiments, the method of treating or preventing human immunodeficiency virus (HIV) in a subject in need thereof comprises administering to the subject one or more any of the polynucleotides, SAMs, LNPs, PNPs, nanoemulsions, expression cassettes, expression vectors, viral vectors, or polypeptides disclosed herein, or any compositions thereof. In some embodiments, the method of treating or preventing human immunodeficiency virus (HIV) in a subject in need thereof comprises administering to the subject (a) one or more SAMs comprising (i) a first polynucleotide sequence comprising nucleotides 1 to 7512 of SEQ ID NO: 522; (ii) a promoter sequence; (iii) a second polynucleotide sequence comprising nucleotides 7537 to 7570 of SEQ ID NO: 522; (iv) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOS: 1-344; (v) a fourth polynucleotide sequence comprising nucleotides 10667 to 10952 of SEQ ID NO: 522; and (vi) a polyA region; or (b) a LNP, PNP, nanoemulsion, or viral vector comprising such SAM. In some embodiments, the method further comprises administering to the subject any of the expression vectors or viral expression vectors disclosed herein, or a LNP, PNP, nanoemulsion, or viral vector comprising such expression vector or viral expression vector disclosed herein. In some embodiments, the method further comprises administering to the subject (a) one or more expression vectors or viral expression vectors comprising (i) the nucleic acid sequence of any one of SEQ ID NOs: 520-521; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521; or (iii) a codon-optimized nucleic acid sequence of the nucleic acid sequence of any one of SEQ ID NOs: 520-521; or (b) a LNP, PNP, nanoemulsion, or viral vector comprising such expression vector or viral expression vector. In some embodiments, the method further comprises administering to the subject (a) one or more expression vectors or viral expression vectors comprising one or more polynucleotides comprising (i) the nucleic acid sequence of any one of SEQ ID NOs: 524-526; (ii) a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526; or (iii) a codon-optimized nucleic acid sequence of the nucleic acid sequence of any one of SEQ ID NOs: 524-526; or (b) a LNP, PNP, nanoemulsion, or viral vector comprising such expression vector or viral expression vector. In some embodiments, the method further comprises administering to the subject (a) one or more expression vectors or viral expression vectors comprising one or more polynucleotides comprising (i) a nucleic acid sequence encoding the polypeptide of any one of SEQ ID NOs: 527-528; (ii) a nucleic acid sequence that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid of SEQ ID NOs: 527-528, wherein percent identity is across the full length of the amino acid sequence of SEQ ID NOs: 527-528; or (iii) a codon-optimized nucleic acid sequence of the nucleic acid sequence encoding the polypeptide of any one of SEQ ID NOs: 527-528; or (b) a LNP, PNP, nanoemulsion, or viral vector comprising such expression vector or viral expression vector. In some embodiments, the subject is infected with HIV-1, is suspected of being infected with HIV-1, or is at risk of being infected with HIV-1. In some embodiments, the subject is chronically infected with HIV-1. In some embodiments, the subject is acutely infected with HIV-1. In some embodiments, the subject has an HIV-1 infection of Fiebig stage IV or earlier, e.g. Fiebig stage III, Fiebig stage II or Fiebig stage I. In some embodiments, administering comprises a route of administration selected from intravenous, intramuscular, intradermal, subcutaneous and mucosal (e.g., buccal, intranasal, intrarectal, intravaginal). In some embodiments, the method comprises administering one or more viral vectors of at a dose range from about 10³ to about 10¹⁵ viral focus forming units (FFU) or plaque forming units (PFU) or infectious units (IU) or viral particles (vp), per administration. In some embodiments, the one or more viral vectors is administered at a dose range from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration. In some embodiments, the one or more the SAMs, LNPs, PNPs, or nanoemulsions disclosed herein is administered at a dose range from about 1 μg to about 1000 μg. In some embodiments, the one or more SAMs, LNPs, PNPs, or nanoemulsions is administered at a dose range from about 15 μg to about 500 μg. In some embodiments, the one or more SAMs, LNPs, PNPs, or nanoemulsions is administered at a dose range from about 1 μg to about 100, 200, 300, 400, 500, 600, 700, 800, 900, or 1000 μg. In some embodiments, any of the methods disclosed herein comprise a prime-boost regimen comprising: (a) administering a priming composition at a first time point and administering one or more boosting compositions at one or more subsequent time points (e.g., prime-boost-boost-boost, etc.); (b) one or more iterations of administering a priming composition at a first time point and administering a boosting composition at a second time point (e.g., prime-boost-prime-boost, etc.); or (c) one more iterations of administering a priming composition at a first time point, administering a boosting composition at one or more subsequent time points, administering the priming composition at a subsequent time point after administration of the boosting composition (e.g., prime-boost-boost-prime, etc.). In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise a polynucleotide encoding the same immunogenic polypeptide. In some embodiments, the priming composition and the boosting composition comprise polynucleotide encoding the different immunogenic polypeptides. In some embodiments, the priming composition and the boosting composition comprise the same one or more polypeptides, polynucleotides, SAMs, LNPs, PNPs, nanoemulsions, or viral vectors. In some embodiments, the priming composition and the boosting composition comprise different polypeptides, polynucleotides, SAMs, LNPs, PNPs, nanoemulsions, or viral vectors. In some embodiments, any of the methods disclosed herein comprise priming with a first polynucleotide, SAM, LNP, PNP, nanoemulsion, or viral vector, and boosting with a second polynucleotide, SAM, LNP, PNP, nanoemulsion, or viral vector. In some embodiments, the prime-boost regimen comprises: (i) priming with a viral expression vector and boosting with a polynucleotide, wherein the polynucleotide is DNA, cDNA, mRNA or self-replicating RNA; (ii) priming with a polynucleotide, wherein the polynucleotide is DNA, cDNA, mRNA or self-replicating RNA, and boosting with a viral expression vector; (iii) priming with a first viral expression vector and boosting with a second viral expression vector, wherein the first and second viral expression vectors are from identical, related or unrelated taxonomical families; (iv) priming with a first replication deficient viral expression vector and boosting with a second replication deficient viral expression vector, wherein the first and second replication deficient viral expression vectors are from identical, related or unrelated taxonomical families; (v) priming with a first attenuated deficient viral expression vector and boosting with a second replication attenuated viral expression vector, wherein the first and second replication attenuated viral expression vectors are from identical, related or unrelated taxonomical families; (vi) priming with a replication deficient viral expression vector and boosting with a replication attenuated viral expression vector; (vii) priming with a replication attenuated viral expression vector and boosting with a replication deficient viral expression vector; (viii) priming with a viral vector and boosting with a composition comprising a SAM; (ix) priming with an adenovirus viral expression vector and boosting with a polynucleotide, wherein the polynucleotide is DNA, cDNA, mRNA or self-replicating RNA; (x) priming with an adenoviral vector and boosting with a composition comprising a SAM; (xi) priming with a ChAd expression vector and boosting with a polynucleotide, wherein the polynucleotide is DNA, cDNA, mRNA or self-replicating RNA; or (xii) priming with a ChAd vector and boosting with a composition comprising a SAM. In some embodiments, the subject is not receiving antiretroviral therapy (ART) or ART is discontinued prior to administration of the one or more polynucleotides, polypeptides, SAMs, LNPs, PNPs, nanoemulsions, expression cassettes, or viral vectors. In some embodiments, ART is discontinued after one or more administrations of the polynucleotides, polypeptides, SAMs, LNPs, PNPs, nanoemulsions, expression cassettes, or viral vectors. In some embodiments, any of the methods disclosed herein further comprise administering to the subject one or more additional therapeutic agents, e.g. two, three, four, or more additional therapeutic agents. In some embodiments, the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs). In some embodiments, the one or more agents that activate latent HIV is one or more latency reversing agents (LRAs). In some embodiments, the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics. In some embodiments, the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, any of the methods disclosed herein further comprise administering GS 9620 (vesatolimod) to the subject. In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, any of the methods disclosed herein further comprise administering lefitolimod (MGN-1703) to the subject. In some embodiments, the one or more bnAbs is an HIV bnAb. In some embodiments, any of the methods disclosed herein further comprise administering one or more bnAbs. In some embodiments, the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses. In some embodiments, the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120. In some embodiments, the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13. In some embodiments, the bnAb is an anti-CD4 bnAb. In some embodiments, the anti-CD4 bnAb is selected from 3BNC117 and VRC01. In some embodiments, the bnAb is an anti-V3 bnAb. In some embodiments, the anti-V3 bnAb is 10-1074. In some embodiments, the one or more interleukin receptor agonists is an agonist of an interleukin selected from IL-2, IL-7, IL-12, IL-15, IL-18, IL-21, IFN-a, IFN-g, GM-CSF and FLT3LG. In some embodiments, the one or more cytokines is selected from the group consisting of IL-2, IL-7, IL-12, IL-15, IL-18, IL-21, IFN-a, IFN-g, GM-CSF, FLT3LG, and combinations and functional variants thereof. In some embodiments, the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2). In some embodiments, one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002. In some embodiments, the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g., less than 400, less than 300, less than 200, less than 100, less than 50, in the absence of anti-retroviral treatment (ART) for at least 6 months, at least 1 year, at least 2 years, at least 3 years, or more.

Additional exemplary embodiments are disclosed below:

1. A polynucleotide comprising any one of the HIV immunogen nucleic acid sequences of SEQ ID NOs: 524-526.

2. A polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the HIV immunogen nucleic acid sequences of SEQ ID NOs: 524-526, wherein percent identity is across the full length of the HIV immunogen nucleic acid sequence of SEQ ID NOs: 524-526.

3. A polynucleotide comprising an HIV immunogen nucleic acid sequence that encodes any one of the HIV immunogen amino acid sequences of SEQ ID NOs: 527-528.

4. A polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to the nucleic acid sequence of a polynucleotide encoding any one of the HIV immunogen amino acid sequences of SEQ ID NOs: 527-528, wherein percent identity is across the full length of the nucleic acid sequence of the polypeptide encoding any one of the HIV immunogen amino acid sequence of SEQ ID NOs: 527-528.

5. The polynucleotide of any one of embodiments 1 to 4, wherein the polynucleotide comprises cDNA or mRNA.

6. A self-amplifying RNA (SAM or saRNA) comprising the polynucleotide of any one of embodiments 1 to 5.

7. The SAM of embodiment 6, wherein the SAM is derived from a virus.

8. The SAM of embodiment 7, wherein the virus is an RNA virus.

9. The SAM of embodiment 8, wherein the RNA virus is positive-sense single-stranded RNA virus.

10. The SAM of any one of embodiments 7 to 9, wherein the virus is selected from a alphavirus, flavivirus, nidovirus, nodamura virus, and picornavirus.

11. The SAM of embodiment 10, wherein the alphavirus is selected from an Old World (OW) alphavirus and New World (NW) alpha virus.

12. The SAM of embodiment 11, wherein the OW alphavirus is selected from Chikunguyna virus (CHIKV), Ross River virus (RRV), Semliki Forest virus (SFV), and Sindbis virus (SINV).

13. The SAM of embodiment 12, wherein the NW alphavirus is selected from Venezuelan equine encephalitis virus (VEEV), eastern equine encephalitis virus (EEEV), and western equine encephalitis virus (WEEV).

14. The SAM of any one of embodiments 7 to 11 and 13, wherein the SAM is derived from Venezuelan equine encephalitis virus (VEEV).

15. The SAM of any one of embodiments 7 to 14, wherein the virus comprises a modified viral genome.

16. The SAM of embodiment 15, wherein the modified viral genome comprises a deletion of one or more genes encoding one or more viral structural proteins.

17. The SAM of embodiment 15 or 16, wherein the modified viral genome is produced by replacing one or more viral structural proteins with the polynucleotide of any one of embodiments 1 to 5.

18. The SAM of any one of embodiments 7 to 17, wherein the virus is replication deficient.

19. A self-amplifying RNA (SAM or saRNA) comprising a polynucleotide, wherein the polynucleotide comprises any one of the nucleic acid sequences of SEQ ID NOs: 522-523.

20. A self-amplifying RNA (SAM or saRNA) comprising a polynucleotide, wherein the polynucleotide comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523.

21. A self-amplifying RNA (SAM or saRNA) comprising a polynucleotide, wherein the polynucleotide comprises any one of the nucleic acid sequences of SEQ ID NOs: 524-526.

22. A self-amplifying RNA (SAM or saRNA) comprising a polynucleotide, wherein the polynucleotide comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526.

23. A self-amplifying RNA (SAM or saRNA) comprising a polynucleotide, wherein the polynucleotide encodes any one of the amino acid sequences of SEQ ID NOs: 527-528.

24. A self-amplifying RNA (SAM or saRNA) comprising a polynucleotide that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid sequences of SEQ ID NOs: 527-528, wherein percent identity is across the full length of the amino acid sequence of SEQ ID NOs: 527-528.

25. A self-amplifying RNA (SAM or saRNA) comprising:
a. a first polynucleotide sequence comprising nucleotides 1 to 7512 of SEQ ID NO: 522;
b. a promoter sequence;
c. a second polynucleotide sequence comprising nucleotides 7537 to 7570 of SEQ ID NO: 522;
d. a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528;
e. a fourth polynucleotide sequence comprising nucleotides 10667 to 10952 of SEQ ID NO: 522; and
f. a polyA region.

26. A self-amplifying RNA (SAM or saRNA) comprising:
a. a first polynucleotide sequence comprising nucleotides 1 to 7512 of SEQ ID NO: 522;
b. a promoter sequence;
c. a second polynucleotide sequence comprising nucleotides 7537 to 7570 of SEQ ID NO: 522;
d. a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344;
e. a fourth polynucleotide sequence comprising nucleotides 10667 to 10952 of SEQ ID NO: 522; and
f. a polyA region.

27. The SAM of embodiment 26, wherein the third polynucleotide sequence encodes two or more polypeptide segments.

28. The SAM of embodiment 26, wherein the third polynucleotide sequence encodes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344.

29. The SAM of embodiment 27 or 28, wherein one or more of the polypeptide segments is abutted or fused to an adjacent polypeptide segment.

30. The SAM of embodiment 27 or 28, wherein one or more of the polypeptide segments is joined to an adjacent polypeptide segment by one or more peptide linkers.

31. The SAM of embodiment 30, wherein the one or more peptide linkers is selected from one or more of a polyalanine linker, a polyglycine linker, a cleavable linker, a flexible linker, a rigid linker, a Nef linking sequence, and combinations thereof.

32. The SAM of embodiment 31, wherein the polyalanine linker comprises or consists of 2 or 3 contiguous alanine residues, e.g. AA, AAA, AAY or AAX, wherein X is any amino acid (e.g. A, C, D, E, F, G, H, I, K, L, M, N, P, Q, R, S, T, V, Y).

33. The SAM of embodiment 31, wherein the flexible linker or polyglycine linker comprises or consists of GG, GGS, GSG or GGGS (SEQ ID NO: 421).

34. The SAM of embodiment 30, wherein the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof.

35. The SAM of embodiment 30, wherein the cleavable linker comprises or consists of a furin recognition/cleavage site selected from the group consisting of RAKR (SEQ ID NO: 381), REKR (SEQ ID NO: 382) and RRKR (SEQ ID NO: 383).

36. The SAM of embodiment 30, wherein the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388).

37. The SAM of embodiment 30, wherein the cleavable linker comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388).

38. The SAM of embodiment 31, wherein the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO: 391), wherein percent identity is across the full length of SEQ ID NOs: 389-391.

39. The SAM of embodiment 31, wherein the Nef linking sequence comprises or consists of an amino acid sequence selected from VHAGPIA (SEQ ID NO: 389), VHAGPVA (SEQ ID NO: 390) and GALDI (SEQ ID NO: 391).

40. The SAM of any one of embodiments 25 to 38, wherein the promoter sequence comprises a polynucleotide sequence of SEQ ID NO: 529.

41. An expression cassette, comprising a polynucleotide of any one of embodiments 1 to 5 or the SAM of any one of embodiments 6 to 40 operably linked to one or more regulatory sequences.

42. The expression cassette of embodiment 41, wherein the polynucleotide is operably linked to and under the control of a constitutive promoter.

43. The expression cassette of embodiment 41, wherein the promoter is selected from a CMV promoter, a CAG promoter, an EF1a promoter, and a 26S sub-genomic promoter.

44. The expression cassette of any one of embodiments 41 to 43, wherein the promoter comprises any of the promoter sequences of SEQ ID NOs: 529-530.

45. A lipid nanoparticle (LNP) comprising the polynucleotide of any one of embodiments 1 to 5, the SAM of any one of embodiments 6 to 40, or the expression cassette of any one of embodiments 41 to 44.

46. The LNP of embodiment 45, wherein the LNP comprises or is synthesized from an ionizable lipid, phospholipid, cholesterol, PEGylated lipid, or any combination thereof.

47. The LNP of embodiment 45 or 46, wherein the LNP comprises or is synthesized from cationic lipid 1,2-dioleoyl-3-timethylammonium-propane (DOTAP).

48. A polymeric nanoparticle (PNP) comprising the polynucleotide of any one of embodiments 1 to 5, the SAM of any one of embodiments 6 to 40, or the expression cassette of any one of embodiments 41 to 44.

49. The PNP of embodiment 48, wherein the PNP comprises or is synthesized from non-degradable polymers, degradable polymers, natural materials, synthetic materials, monomers, dendrimers, or any combination thereof.

50. The PNP of embodiment 48 or 49, wherein the PNP comprises or is synthesized from poly(ethylene glycol) (PEG), poly(dimethylsiloxane) (PDMS), polyethyleneimine (PEI), poly(amidoamine) (PAMAM), poly(dimethylaminoethyl) acrylate (pDMAEA), orthenine-derived dendrimers, or any combination thereof.

51. The PNP of any one of embodiments 48 to 50, wherein the PNP comprises or is synthesized from mannosylated-PEI polyplexes.

52. The PNP of any one of embodiments 48 to 51, wherein the PNP comprises or is synthesized from a bioreducible, linear, cationic polymer (pABOL).

53. A nanoemulsion comprising the polynucleotide of any one of embodiments 1 to 5, the SAM of any one of embodiments 6 to 40, or the expression cassette of any one of embodiments 41 to 44.

54. The nanoemulsion of embodiment 53, wherein the nanoemulsion is a water-in-oil emulsion.

55. The nanoemulsion of embodiment 53, wherein the nanoemulsion comprises squalene, sorbitan trioleate, polysorbate 80, DOTAP, or any combination thereof.

56. An expression vector comprising one or more polynucleotides of any one of embodiments 1 to 5, the SAM of any one of embodiments 6 to 40, or the expression cassette of any one of embodiments 41 to 43.

57. The expression vector of embodiment 56, wherein the vector is a plasmid vector, a bacterial vector or a viral expression vector.

58. The expression vector of embodiment 56 or 57, wherein the vector is a viral expression vector.

59. The expression vector of embodiment 58, wherein the viral expression vector is a DNA virus or an RNA virus.

60. The expression vector of any one of embodiments 58 to 59, wherein the viral expression vector is replication defective, replication deficient, replication attenuated or replication competent.

61. The expression vector of any one of embodiments 58 to 60, wherein the viral expression vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus.

62. The expression vector of any one of embodiments 58 to 61, wherein the viral expression vector is from a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus).

63. The expression vector of any one of embodiments 58 to 62, wherein the viral expression vector is an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV).

64. The expression vector of embodiment 63, wherein the viral expression vector is an arenavirus vector selected from Lymphocytic choriomeningitis mammarenavirus (LCMV) or Cali mammarenavirus (a.k.a. Pichinde mammarenavirus or Pichinde arenavirus).

65. The expression vector of embodiment 63 or 64, wherein the arenavirus vector comprises a bi-segmented genome.

66. The expression vector of embodiment 63 or 64, wherein the arenavirus vector comprises a tri-segmented genome.

67. The expression vector of any one of embodiments 58 to 62, wherein the viral expression vector is a human adenovirus or a simian adenovirus.

68. The expression vector of embodiment 67, wherein the simian adenovirus is selected from a chimpanzee adenovirus, a gorilla adenovirus, and a rhesus adenovirus.

69. The expression vector of any one of embodiments 58 to 62 and 67, wherein the viral expression vector is an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (ChAd), gorilla adenovirus, and rhesus adenovirus.

70. The expression vector of embodiment 69, wherein the ChAd is selected from ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), and ChAd147 (AdC147).

71. The expression vector of embodiment 70, wherein the ChAd is ChAd68.

72. The expression vector of embodiment 69, wherein the gorilla adenovirus is selected from GC44, GC45, and GC46.

73. The expression vector of embodiment 69, wherein the rhesus adenovirus (RhAd) is selected from RhAd51, RhAd52, RhAd53, RhAd54, RhAd55, RhAd56, RhAd57, RhAd58, RhAd59, RhAd60, RhAd61, RhAd62, RhAd63, RhAd64, RhAd65 and RhAd66.

74. The expression vector of any one of embodiments 67 to 73, wherein the human adenovirus or simian adenovirus comprises a modification of one or more adenoviral genes.

75. The expression vector of embodiment 74, wherein the one or more adenoviral genes are selected from E1, E3, and E4ORF2-4.

76. The expression vector of embodiment 74 or 75, wherein the modification comprises a deletion, substitution, or insertion of one or more nucleotides into the one or more adenoviral genes.

77. The expression vector of any one of embodiments 74 to 76, wherein the human adenovirus or simian adenovirus comprises a deletion of at least a portion of the E1 region.

78. The expression vector of embodiment 77, wherein the polynucleotide is inserted into the deletion of at least a portion of the E1 region.

79. The expression vector of any one of embodiments 56 to 78, wherein the expression vector comprises any one of the nucleic acid sequences of SEQ ID NOs: 520-521.

80. The expression vector of any one of embodiments 56 to 78, wherein the expression vector comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521.

81. An expression vector comprising any one of the nucleic acid sequences of SEQ ID NOs: 524-526.

82. An expression vector comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526.

83. An expression vector comprising a polynucleotide sequence that encodes for any one of the amino acid sequences of SEQ ID NOs: 527-528.

84. An expression vector comprising a polynucleotide sequence that encodes for polynucleotide that is at least 80%, 85%, 86%, 87%, 88%, 89%, 90%, 91%, 92%, 93%, 94%, 95%, 96%, 97%, 98%, 99%, or 100% identical to any one of the amino acid sequences of SEQ ID NOs: 527-528, wherein percent identity is across the full length of the amino acid sequence of SEQ ID NOs: 527-528.

85. An expression vector comprising:
a. a first polynucleotide sequence comprising nucleotides 1 to 593 of SEQ ID NO: 520;
b. a promoter sequence;
c. a second polynucleotide sequence comprising nucleotides 1196 to 1255 of SEQ ID NO: 520;
d. a third polynucleotide sequence encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528;
e. a fourth polynucleotide sequence comprising nucleotides 4352 to 4395 of SEQ ID NO: 520;
f. a polyA sequence; and
g. a fifth polynucleotide sequence comprising nucleotides 4620 to 32350 of SEQ ID NO: 520.

86. An expression vector comprising:
a. a first polynucleotide sequence comprising nucleotides 1 to 593 of SEQ ID NO: 520;
b. a promoter sequence;
c. a second polynucleotide sequence comprising nucleotides 1196 to 1255 of SEQ ID NO: 520;
d. a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344;
e. a fourth polynucleotide sequence comprising nucleotides 4352 to 4395 of SEQ ID NO: 520;
f. a polyA sequence; and
g. a fifth polynucleotide sequence comprising nucleotides 4620 to 32350 of SEQ ID NO: 520.

87. The expression vector of embodiment 86, wherein the third polynucleotide sequence encodes two or more polypeptide segments.

88. The expression vector of embodiment 86, wherein the third polynucleotide sequence encodes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344 89. The expression vector of embodiment 87 or 88, wherein one or more of the polypeptide segments is abutted or fused to an adjacent polypeptide segment.

90. The expression vector of embodiment 87 or 88, wherein one or more of the polypeptide segments is joined to an adjacent polypeptide segment by one or more peptide linkers.

91. The expression vector of embodiment 90, wherein the one or more peptide linkers is selected from one or more of a polyalanine linker, a polyglycine linker, a cleavable linker, a flexible linker, a rigid linker, a Nef linking sequence, and combinations thereof.

92. The expression vector of embodiment 91, wherein the polyalanine linker comprises or consists of 2 or 3 contiguous alanine residues, e.g. AA, AAA, AAY or AAX, wherein X is any amino acid (e.g. A, C, D, E, F, G, H, I, K, L, M, N, P, Q, R, S, T, V, Y).

93. The expression vector of embodiment 91, wherein the flexible linker or polyglycine linker comprises or consists of GG, GGS, GSG or GGGS (SEQ ID NO: 421).

94. The expression vector of embodiment 90, wherein the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof.

95. The expression vector of embodiment 90, wherein the cleavable linker comprises or consists of a furin recognition/cleavage site selected from the group consisting of RAKR (SEQ ID NO: 381), REKR (SEQ ID NO: 382) and RRKR (SEQ ID NO: 383).

96. The expression vector of embodiment 90, wherein the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388), wherein percent identity is across the full length of SEQ ID NOs: 384-388.

97. The expression vector of embodiment 90, wherein the cleavable linker comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388).

98. The expression vector of embodiment 91, wherein the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO: 391), wherein percent identity is across the full length of SEQ ID NOs: 389-391.

99. The expression vector of embodiment 91, wherein the Nef linking sequence comprises or consists of an amino acid sequence selected from VHAGPIA (SEQ ID NO: 389), VHAGPVA (SEQ ID NO: 390) and GALDI (SEQ ID NO: 391).

100. The expression vector of any one of embodiments 85 to 98, wherein the promoter sequence comprises a polynucleotide sequence of SEQ ID NO: 529.

101. A viral vector comprising one or more polynucleotides of any one of embodiments 1 to 5 the SAM of any one of embodiments 6 to 40, the expression cassette of any one of embodiments 41 to 44, or the expression vector of any one of embodiments 58 to 100.

102. The viral vector of embodiment 101, wherein the viral vector is derived from or based on a DNA virus or an RNA virus.

103. The viral vector of embodiment 101 or 102, wherein the viral vector is replication defective, replication deficient, replication attenuated or replication competent.

104. The viral vector of any one of embodiments 101 to 103, wherein the viral expression vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus.

105. The viral vector of any one of embodiments 101 to 104, wherein the viral vector is derived from or based on a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus).

106. The viral vector of any one of embodiments 101 to 105, wherein the viral vector is derived from or based on an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV).

107. The viral vector of embodiment 106, wherein the viral vector is derived from or based on an arenavirus vector selected from Lymphocytic choriomeningitis mammarenavirus (LCMV) or Cali mammarenavirus (a.k.a. Pichinde mammarenavirus or Pichinde arenavirus).

108. The viral vector of embodiment 106 or 107, wherein the arenavirus vector comprises a bi-segmented genome.

109. The viral vector of embodiment 106 or 107, wherein the arenavirus vector comprises a tri-segmented genome.

110. The viral vector of any one of embodiments 101 to 105, wherein the viral vector is a human adenovirus or a simian adenovirus.

111. The viral vector of embodiment 110, wherein the simian adenovirus is selected from a chimpanzee adenovirus, a gorilla adenovirus, and a rhesus adenovirus.

112. The viral vector of any one of embodiments 101 to 105 and 110, wherein the viral vector is derived from or based on an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (ChAd), gorilla adenovirus, and rhesus adenovirus.

113. The viral vector of embodiment 112, wherein the ChAd is selected from ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), and ChAd147 (AdC147).

114. The viral vector of embodiment 113, wherein the ChAd is ChAd68.

115. The viral vector of embodiment 114, wherein the gorilla adenovirus is selected from GC44, GC45, and GC46.

116. The viral vector of embodiment 112, wherein the rhesus adenovirus (RhAd) is selected from RhAd51, RhAd52, RhAd53, RhAd54, RhAd55, RhAd56, RhAd57, RhAd58, RhAd59, RhAd60, RhAd61, RhAd62, RhAd63, RhAd64, RhAd65 and RhAd66.

117. The viral vector of any one of embodiments 110 to 116, wherein the human adenovirus or simian adenovirus comprises a modification of one or more adenoviral genes.

118. The viral vector of embodiment 117, wherein the one or more adenoviral genes are selected from E1, E3, and E4ORF2-4.

119. The viral vector of embodiment 117 or 118, wherein the modification comprises a deletion, substitution, or insertion of one or more nucleotides into the one or more adenoviral genes.

120. The viral vector of any one of embodiments 117 to 119, wherein the human adenovirus or simian adenovirus comprises a deletion of at least a portion of the E1 region.

121. The viral vector of embodiment 120, wherein the polynucleotide is inserted into the deletion of at least a portion of the E1 region.

122. The viral vector of any one of embodiments 101 to 121, wherein the viral vector comprises any one of the nucleic acid sequences of SEQ ID NOs: 520-521.

123. The viral vector of any one of embodiments 101 to 121, wherein the vector comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521.

124. A host cell comprising one or more polynucleotides of any one of embodiments 1 to 5, one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, one or more nanoemulsions of any one of embodiments 53 to 55, one or more expression vectors of any one of embodiments 56 to 100, or one or more viral vectors of any one of embodiments 101 to 123.

125. The host cell of embodiment 124, wherein the one or more polynucleotides are not integrated into the host cell genome, e.g., are episomal.

126. The host cell of embodiment 124, wherein the one or more polynucleotides are integrated into the host cell genome.

127. The host cell of any one of embodiments 124 to 126, wherein the host cell is a mammalian cell.

128. The host cell of embodiment 127, wherein the mammalian cell is a human cell.

129. The host cell of embodiment 127, wherein the mammalian cell is not a human cell.

130. The host cell of any one of embodiments 124 to 129, wherein the host cell is in vitro.

131. The host cell of any one of embodiments 124 to 129, wherein the host cell is in vivo.

132. A polypeptide comprising any of the HIV immunogen amino acid sequences of SEQ ID NOs: 527-528.

133. A polypeptide comprising an amino nucleic acid sequence that is at least 80%, 85%, 86%, 87%, 88%, 89%, 90%, 91%, 92%, 93%, 94%, 95%, 96%, 97%, 98%, 99%, or 100% identical any of the HIV immunogen amino acid sequences of SEQ ID NOs: 527-528, wherein percent identity is across the full length of the HIV immunogen amino acid sequence of SEQ ID NOs: 527-528.

134. A composition comprising (a) one or more polynucleotides of any one of embodiments 1 to 5, one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, one or more nanoemulsions of any one of embodiments 53 to 55, one or more expression vectors of any one of embodiments 56 to 100, one or more viral vectors of any one of embodiments 101 to 123, or one or more polypeptides of embodiment 132 or 133; and (b) a pharmaceutically acceptable diluent, carrier or excipient.

135. The composition of embodiment 134, comprising two or more polynucleotides of any one of embodiments 1 to 5, two or more SAMs of any one of embodiments 6 to 40, two or more expression cassettes of any one of embodiments 41 to 44, two or more LNPs of any one of embodiments 45 to 47, two or more PNPs of any one of embodiments 48 to 52, two or more nanoemulsions of any one of embodiments 53 to 55, or two or more expression vectors of any one of embodiments 56 to 100, two or more viral vectors of any one of embodiments 101 to 123, or two or more polypeptides of embodiment 132 or 133.

136. A composition comprising (a) one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, or one or more nanoemulsions of any one of embodiments 53 to 55; and (b) one or more expression vectors of any one of embodiments 56 to 100 or one or more viral vectors of any one of embodiments 101 to 123.

137. A composition comprising (a) one or more polypeptides of embodiment 132 or 133; and (b) a pharmaceutically acceptable diluent, carrier or excipient.

138. The composition of embodiment 137, comprising two or more polypeptides of embodiment 132 or 133.

139. The composition of embodiment 136, further comprising a pharmaceutically acceptable diluent, carrier or excipient.

140. The composition of any one of embodiments 134 to 139, further comprising one or more of an adjuvant, an immunostimulator, a detergent, a micelle-forming agent, and an oil.

141. The composition of embodiment 140, wherein the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein.

142. The composition of embodiment 141, wherein the cytokine selected from IL-2, IL-7, IL-12, IL-15, IL-18, IL-21, IFN-α, IFN-γ, GM-CSF, FLT3LG, and combinations and functional variants thereof.

143. The composition of embodiment 141, comprising the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102).

144. The composition of any one of embodiments 134 to 143, wherein the composition is formulated for administration via a route selected from the group consisting of intravenous, intramuscular, intradermal, subcutaneous and mucosal (e.g. buccal, intranasal, intrarectal, intravaginal).

145. The composition of any one of embodiments 134 to 143, wherein the composition is formulated as a liquid.

146. The composition of any one of embodiments 134 to 143, wherein the composition is lyophilized.

147. A kit comprising one or more components selected from one or more polynucleotides of any one of embodiments 1 to 5, one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, one or more nanoemulsions of any one of embodiments 53 to 55, one or more expression vectors of any one of embodiments 56 to 100, or one or more viral vectors of any one of embodiments 101 to 123, or one or more polypeptides of embodiment 132 or 133.

148. A kit comprising (a) a first component selected from one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, or one or more nanoemulsions of any one of embodiments 53 to 55; and (b) second component comprising one or more expression vectors of any one of embodiments 56 to 100 or one or more viral vectors of any one of embodiments 101 to 123.

149. A kit comprising one or more unitary doses of one or more polynucleotides of any one of embodiments 1 to 5, one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, one or more nanoemulsions of any one of embodiments 53 to 55, one or more expression vectors of any one of embodiments 56 to 100, or one or more viral vectors of any one of embodiments 101 to 123, or one or more polypeptides of embodiment 132 or 133.

150. A kit comprising (a) one or more unitary doses of one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, or one or more nanoemulsions of any one of embodiments 53 to 55; and (b) one or more unitary doses of one or more expression vectors of any one of embodiments 56 to 100 or one or more viral vectors of any one of embodiments 101 to 123.

151. A kit comprising one or more unitary doses of one or more polypeptides of embodiment 132 or 133.

152. The kit of embodiment 147, wherein the one or more components are in a single container.

153. The kit of embodiment 147, wherein the one or more components are in two or more separate containers.

154. The kit of embodiment 148, wherein the first and second components are in a single container.

155. The kit of embodiment 148, wherein the first and second components are in two or more separate containers.

156. The kit of any one of embodiments 149 to 151, wherein the one or more unitary doses are in a single container.

157. The kit of any one of embodiments 149 to 151, wherein the one or more unitary doses are in two or more separate containers.

158. The kit of any one of embodiments 152 to 157, comprising one or more containers selected from the group consisting of vials, ampules and pre-loaded syringes.

159. The kit of any one of embodiments 152 to 158, comprising one or more containers comprising the one or more SAMs, one or more LNPs, one or more PNPs, one or more nanoemulsions, one or more vectors, or one or more polypeptides in an aqueous solution.

160. The kit of any one of embodiments 149, 150, and 156 to 159, wherein the one or more unitary doses are the same.

161. The kit of any one of embodiments 149, 150, and 156 to 159, wherein the one or more unitary doses are the different.

162. The kit of any one of embodiments 149, 150, and 156 to 161, comprising one or more unitary doses of one or more viral vectors of any one of embodiments 56 to 80, wherein the unitary doses are in the range of about 10³ to about 10¹⁵ viral focus forming units (FFU) or plaque forming units (PFU) or infectious units (IU) or viral particles (vp).

163. The kit of embodiment 162, wherein the unitary doses of the one or more viral vectors is from about 10⁴ to about 10⁷ viral FFU or PFU or IU or vp.

164. The kit of embodiment 162, wherein the unitary doses of the one or more viral vectors is from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp.

165. The kit of any one of embodiments 149, 150, and 156 to 161, comprising one or more unitary doses of one or more SAMs of any one of embodiments 6 to 40, wherein the unitary doses are in the range of about 1 μg to about 1000 μg.

166. The kit of embodiment 165, wherein the unitary doses of the one or more SAMs is from about 15 μg to about 500 μg.

167. The kit of embodiment 165, wherein the unitary doses of the one or more SAMs is from about 1 μg to about 100, 200, 300, 400, 500, 600, 700, 800, 900, or 1000 μg.

168. The composition of any one of embodiments 134 to 146 or the kit of any one of embodiments 147 to 167, further comprising one or more unitary doses of one or more additional therapeutic agents.

169. The composition or kit of embodiment 168, wherein the one or more additional therapeutic agents is selected from one or more agents that activate latent HIV, one or more agonists or activators of one or more toll-like receptors (TLRs), one or more interleukin receptor agonists, one or more cytokines, one or more receptor agonists, one or more inhibitors of a T-cell inhibitory immune checkpoint protein or receptor, one or more agonists, activators or stimulator of a T-cell stimulatory immune checkpoint protein or receptor, one or more inhibitors of CD47, one or more immune-based therapies, one or more broadly neutralizing antibodies (bnAbs), and one or more antiviral agents.

170. The composition or kit of embodiment 169, wherein the one or more agents that activate latent HIV is one or more latency reversing agents (LRAs).

171. The composition or kit of embodiment 170, wherein the one or more LRAs is selected from the group consisting of agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics.

172. The composition or kit of embodiment 169, wherein the TLR agonist or activator is selected from the group consisting of a TLR2 agonist, a TLR3 agonist, a TLR4 agonist, a TLR5 agonist, a TLR7 agonist, a TLR8 agonist and a TLR9 agonist.

173. The composition or kit of embodiment 172, wherein the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist).

174. The composition or kit of embodiment 173, further comprising GS 9620 (vesatolimod).

175. The composition or kit of embodiment 172 wherein the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042.

176. The composition or kit of embodiment 175, further comprising lefitolimod (MGN-1703).

177. The composition or kit of embodiment 169, wherein the interleukin is selected from IL-2, IL-7, IL-12, IL-15, IL-18, IL-21, IFN-a, IFN-g, GM-CSF, FLT3LG.

178. The composition or kit of embodiment 169, wherein the one or more cytokines selected from the group consisting of IL-2, IL-7, IL-12, IL-15, IL-18, IL-21, IFN-a, IFN-g, GM-CSF, FLT3LG, and combinations and functional variants thereof.

179. The composition or kit of embodiment 169, wherein the receptor agonist is an agonist of one or more receptors selected from fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2).

180. The composition or kit of embodiment 169, wherein the one or more inhibitors of a T-cell inhibitory immune checkpoint protein or receptor inhibits a checkpoint protein or receptor selected from the group consisting of CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1).

181. The composition or kit of embodiment 169, wherein the one or more agonists, activators or stimulators of a T-cell stimulatory immune checkpoint protein or receptor agonizes, activates or stimulates a T-cell stimulatory immune checkpoint protein or receptor selected from the group consisting of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155).

182. The composition or kit of embodiment 180, wherein the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002.

183. The composition or kit of embodiment 169, 180 or 182, further comprising ipilimumab.

184. The composition or kit of embodiment 180, wherein the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 ( ) JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181.

185. The composition or kit of embodiment 169, 180 or 184, further comprising pembrolizumab.

186. The composition or kit of embodiment 169, 180 or 184, further comprising nivolumab.

187. The composition or kit of embodiment 169, 180 or 184, further comprising AB122 (zimberelimab).

188. The composition or kit of embodiment 169, wherein the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors.

189. The composition or kit of embodiment 169, wherein the one or more immune-based therapies is selected from the group consisting of interferon alfa, interferon alfa-2b, interferon alfa-n3, pegylated interferon alfa, interferon gamma, fms related tyrosine kinase 3 (FLT3) agonists, gepon, normferon, peginterferon alfa-2a, peginterferon alfa-2b, and RPI-MN.

190. The composition or kit of embodiment 189, wherein the FLT3 agonist is selected from GS-3583 and CDX-301.

191. The composition or kit of embodiment 169, 189 or 190, further comprising GS-3583.

192. The composition or kit of embodiment 169, 189 or 190, further comprising CDX-301.

193. The composition or kit of embodiment 169, further comprising one or more bnAbs.

194. The composition or kit of embodiment 169, wherein the one or more bnAbs is an HIV bnAb.

195. The composition or kit of embodiment 194, wherein the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses.

196. The composition or kit of any one of embodiments 169 and 193 to 195, wherein the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120.

197. The composition or kit of any one of embodiments 169 and 193 to 196, wherein the bnAb is selected from 10-1074, 10E8, 12A12, 12A21, 2F5, 2G12, 3BC176, 3BNC117, 3BNC55, 3BNC60, 3BNC62, 447-52D, 4E10, 5H/I1-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13.

198. The composition or kit of any one of embodiments 169 and 193 to 197, wherein the bnAb is an anti-CD4 bnAb.

199. The composition or kit of embodiment 198, wherein the anti-CD4 bnAb is selected from 3BNC117 and VRC01.

200. The composition or kit of any one of embodiments 169 and 193 to 197, wherein the bnAb is an anti-V3 bnAb.

201. The composition or kit of embodiment 200, wherein the anti-V3 bnAb is 10-1074.

202. A method for eliciting an immune response to human immunodeficiency virus (HIV) in a subject in need thereof, comprising administering to the subject one or more polynucleotides of any one of embodiments 1 to 5, one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, one or more nanoemulsions of any one of embodiments 53 to 55, one or more expression vectors of any one of embodiments 56 to 100, one or more viral vectors of any one of embodiments 101 to 123, one or more polypeptides of embodiment 134 to 146, or one or more compositions of any one of embodiments 134 to 146.

203. A method of treating or preventing human immunodeficiency virus (HIV) in a subject in need thereof, comprising administering to the subject one or more polynucleotides of any one of embodiments 1 to 5, one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, one or more nanoemulsions of any one of embodiments 53 to 55, one or more expression vectors of any one of embodiments 56 to 100, one or more viral vectors of any one of embodiments 101 to 123, one or more polypeptides of embodiment 134 to 146, or one or more compositions of any one of embodiments 134 to 146.

204. A method for eliciting an immune response to human immunodeficiency virus (HIV) in a subject in need thereof, comprising administering to the subject (a) one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, one or more nanoemulsions of any one of embodiments 53 to 55; and (b) one or more expression vectors of any one of embodiments 56 to 100 or one or more viral vectors of any one of embodiments 101 to 123.

205. A method of treating or preventing human immunodeficiency virus (HIV) in a subject in need thereof, comprising administering to the subject (a) one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, one or more nanoemulsions of any one of embodiments 53 to 55; and (b) one or more expression vectors of any one of embodiments 56 to 100 or one or more viral vectors of any one of embodiments 101 to 123.

206. The method of any one of embodiments 202 to 205, wherein the subject is infected with HIV-1, is suspected of being infected with HIV-1, or is at risk of being infected with HIV-1.

207. The method of any one of embodiments 202 to 206, wherein the subject is chronically infected with HIV-1.

208. The method of any one of embodiments 202 to 207, wherein the subject is acutely infected with HIV-1.

209. The method of any one of embodiments 202 to 208, wherein the subject has an HIV-1 infection of Fiebig stage IV or earlier, e.g., Fiebig stage III, Fiebig stage II or Fiebig stage I.

210. The method of any one of embodiments 202 to 209, wherein administering comprises a route of administration selected from intravenous, intramuscular, intradermal, subcutaneous and mucosal (e.g., buccal, intranasal, intrarectal, intravaginal).

211. The method of any one of embodiments 202 to 210, wherein the method comprises administering one or more viral vectors of any one of embodiments 56 to 80 at a dose range from about 10³ to about 10¹⁵ viral focus forming units (FFU) or plaque forming units (PFU) or infectious units (IU) or viral particles (vp), per administration.

212. The method of embodiment 211, wherein the one or more viral vectors is administered at a dose range from about 10⁴ to about 10⁷ viral FFU or PFU or IU or vp, e.g., from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration.

213. The method of any one of embodiments 202 to 212, wherein the method comprises administering one or more SAMs of any one of embodiments 6 to 40, one or more LNPs of any one of embodiments 45 to 47, one or more PNPs of any one of embodiments 48 to 52, one or more nanoemulsions of any one of embodiments 53 to 55 at a dose range from about 1 μg to about 1000 μg.

214. The method of any one of embodiments 202 to 213, wherein the one or more SAMs, LNPs, PNPs, or nanoemulsions is administered at a dose range from about 15 μg to about 500 ug.

215. The method of any one of embodiments 202 to 214, wherein the one or more SAMs, LNPs, PNPs, or nanoemulsions is administered at a dose range from about 1 μg to about 100, 200, 300, 400, 500, 600, 700, 800, 900, or 1000 μg.

216. The method of any one of embodiments 202 to 215, comprising a prime-boost regimen comprising:
(i) administering a priming composition at a first time point and administering one or more boosting compositions at one or more subsequent time points (e.g., prime-boost-boost-boost, etc.);
(ii) one or more iterations of administering a priming composition at a first time point and administering a boosting composition at a second time point (e.g., prime-boost-prime-boost, etc.); or
(iii) one more iterations of administering a priming composition at a first time point, administering a boosting composition at one or more subsequent time points, administering the priming composition at a subsequent time point after administration of the boosting composition (e.g., prime-boost-boost-prime, etc.).

217. The method of embodiment 216, wherein the administrations of the priming composition and the one or more boosting compositions are spaced at least 1 week, 2 weeks, 3 weeks or 1 month apart, e.g., at least 2, 3, 4, 5 or 6 months, apart.

218. The method of any one of embodiments 202 to 217, wherein the priming composition and the boosting composition comprise a polynucleotide encoding the same immunogenic polypeptide.

219. The method of any one of embodiments 202 to 217, wherein the priming composition and the boosting composition comprise polynucleotide encoding the different immunogenic polypeptides.

220. The method of any one of embodiments 202 to 217, wherein the priming composition and the boosting composition comprise the same one or more polypeptides, polynucleotides, SAMs, LNPs, PNPs, nanoemulsions, or viral vectors.

221. The method of any one of embodiments 202 to 217, wherein the priming composition and the boosting composition comprise different polypeptides, polynucleotides, SAMs, LNPs, PNPs, nanoemulsions, or viral vectors.

222. The method of embodiment 219 or 221, comprising priming with a first polynucleotide, SAM, LNP, PNP, nanoemulsion, or viral vector, and boosting with a second polynucleotide, SAM, LNP, PNP, nanoemulsion, or viral vector.

223. The method of any one of embodiments 202 to 222, wherein the prime-boost regimen comprises:
a. Priming with a viral expression vector and boosting with a polynucleotide, wherein the polynucleotide is DNA, cDNA, mRNA or self-replicating RNA;
b. Priming with a polynucleotide, wherein the polynucleotide is DNA, cDNA, mRNA or self-replicating RNA, and boosting with a viral expression vector;
c. Priming with a first viral expression vector and boosting with a second viral expression vector, wherein the first and second viral expression vectors are from identical, related or unrelated taxonomical families;
d. Priming with a first replication deficient viral expression vector and boosting with a second replication deficient viral expression vector, wherein the first and second replication deficient viral expression vectors are from identical, related or unrelated taxonomical families;
e. Priming with a first attenuated deficient viral expression vector and boosting with a second replication attenuated viral expression vector, wherein the first and second replication attenuated viral expression vectors are from identical, related or unrelated taxonomical families;
f. Priming with a replication deficient viral expression vector and boosting with a replication attenuated viral expression vector;
g. Priming with a replication attenuated viral expression vector and boosting with a replication deficient viral expression vector;
h. Priming with a viral vector and boosting with a composition comprising a SAM;
i. Priming with an adenovirus viral expression vector and boosting with a polynucleotide, wherein the polynucleotide is DNA, cDNA, mRNA or self-replicating RNA;
j. Priming with an adenoviral vector and boosting with a composition comprising a SAM;
k. Priming with a ChAd expression vector and boosting with a polynucleotide, wherein the polynucleotide is DNA, cDNA, mRNA or self-replicating RNA; or
l. Priming with a ChAd vector and boosting with a composition comprising a SAM.

224. The method of any one of embodiments 202 to 223, wherein the subject is not receiving antiretroviral therapy (ART) or ART is discontinued prior to administration of the one or more polynucleotides, polypeptides, SAMs, LNPs, PNPs, nanoemulsions, expression cassettes, or viral vectors.

225. The method of any one of embodiments 202 to 224, wherein ART is discontinued after one or more administrations of the polynucleotides, polypeptides, SAMs, LNPs, PNPs, nanoemulsions, expression cassettes, or viral vectors.

226. The method of any one of embodiments 202 to 225, further comprising administering to the subject one or more additional therapeutic agents, e.g., two, three, four, or more additional therapeutic agents.

227. The method of embodiment 226, wherein the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs).

228. The method of embodiment 227, wherein the one or more agents that activate latent HIV is one or more latency reversing agents (LRAs).

229. The method of embodiment 228, wherein the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics.

230. The method of embodiment 227, wherein the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist.

231. The method of embodiment 230, wherein the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist).

232. The method of embodiment 231, further comprising administering GS 9620 (vesatolimod) to the subject.

233. The method of embodiment 230, wherein the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042.

234. The method of embodiment 233, further comprising administering lefitolimod (MGN-1703) to the subject.

235. The method of any one of embodiments 227 to 234, further comprising administering one or more bnAbs to the subject.

236. The method of embodiment 227 or 235, wherein the one or more bnAbs is an HIV bnAb.

237. The method of embodiment 236, wherein the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses.

238. The method of any one of embodiments 227 and 235 to 237, wherein the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120.

239. The method of any one of embodiments 227 and 235 to 238, wherein the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03,

240. The method of any one of embodiments 227 and 235 to 239, wherein the bnAb is an anti-CD4 bnAb.

241. The method of embodiment 240, wherein the anti-CD4 bnAb is selected from 3BNC117 and VRC01.

242. The method of any one of embodiments 227 and 235 to 239, wherein the bnAb is an anti-V3 bnAb.

243. The method of embodiment 242, wherein the anti-V3 bnAb is 10-1074.

244. The method of embodiment 227, wherein the one or more interleukin receptor agonists is an agonist of an interleukin selected from IL-2, IL-7, IL-12, IL-15, IL-18, IL-21, IFN-a, IFN-g, GM-CSF and FLT3LG.

245. The method of embodiment 227, wherein the one or more cytokines is selected from the group consisting of IL-2, IL-7, IL-12, IL-15, IL-18, IL-21, IFN-a, IFN-g, GM-CSF, FLT3LG, and combinations and functional variants thereof.

246. The method of embodiment 227, wherein the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2).

247. The method of embodiment 227, wherein one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1).

248. The method of embodiment 227, wherein the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155).

249. The method of embodiment 247, wherein the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002.

250. The method of embodiment 247 or 249, further comprising administering ipilimumab to the subject.

251. The method of embodiment 247, wherein the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181.

252. The method of embodiment 247 or 251, further comprising administering pembrolizumab to the subject.

253. The method of embodiment 247 or 251, further comprising administering nivolumab, to the subject.

254. The method of embodiment 247 or 251, further comprising administering AB122 (zimberelimab) to the subject.

255. The method of embodiment 227, wherein the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors.

256. The method of embodiment 227, wherein the one or more immune-based therapies is selected from the group consisting of interferon alfa, interferon alfa-2b, interferon alfa-n3, pegylated interferon alfa, interferon gamma; fms related tyrosine kinase 3 (FLT3) agonists, gepon, normferon, peginterferon alfa-2a, peginterferon alfa-2b, and RPI-MN.

257. The method of embodiment 256, wherein the FLT3 agonist is selected from GS-3583 and CDX-301.

258. The method of embodiment 227, 256, or 257, wherein the method further comprises administering GS-3583 to the subject.

259. The method of embodiment 227, 256, or 257, wherein the method further comprises administering CDX-301 to the subject.

260. The method of any one of embodiments 202 to 259, wherein after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more.

261. The method of any one of embodiments 202 to 260, wherein, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g., less than 400, less than 300, less than 200, less than 100, less than 50, in the absence of anti-retroviral treatment (ART) for at least 6 months, at least 1 year, at least 2 years, at least 3 years, or more.

262. One or more polynucleotides of any one of embodiments 1 to 5, one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, one or more nanoemulsions of any one of embodiments 53 to 55, one or more expression vectors of any one of embodiments 56 to 100, one or more viral vectors of any one of embodiments 101 to 123, one or more polypeptides of embodiment 132 or 133, or one or more compositions of any one of embodiments 134 to 146, for use in eliciting an immune response to human immunodeficiency virus (HIV) in a subject in need thereof.

263. One or one or more polynucleotides of any one of embodiments 1 to 5, one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, one or more nanoemulsions of any one of embodiments 53 to 55, one or more expression vectors of any one of embodiments 56 to 100, one or more viral vectors of any one of embodiments 101 to 123, one or more polypeptides of embodiment 132 or 133, or one or more compositions of any one of embodiments 134 to 146, for use in treating or preventing human immunodeficiency virus (HIV) in a subject in need thereof.

264. One or more polynucleotides of any one of embodiments 1 to 5, one or more SAMs of any one of embodiments 6 to 40, one or more expression cassettes of any one of embodiments 41 to 44, one or more LNPs of any one of embodiments 45 to 47, one or more PNPs of any one of embodiments 48 to 52, one or more nanoemulsions of any one of embodiments 53 to 55, one or more expression vectors of any one of embodiments 56 to 100, one or more viral vectors of any one of embodiments 101 to 123, one or more polypeptides of embodiment 132 or 133, or one or more compositions of any one of embodiments 134 to 146, for use as a medicament.

### EXAMPLES

The following examples are offered to illustrate, but not to limit the claimed invention.

### Example 1

### Illustrated Implementation of the Conservation Analysis and Conserved Walking Analysis (CWA) to Generate a Bivalent Vaccine Construct

This Example describes the design of population-based bivalent polypeptide constructs by a specific implementation of the Conservation Analysis and CWA to generate a bivalent vaccine construct based on conserved protein regions encoded by the HIV-1 Env, Gag, Nef and/or Pol genes.

First, the method identifies a set of all conserved regions in a viral proteome for a selected set of viral genes. In this example, the set of viral genes consisted of HIV-1 Gag, Pol, and Nef.

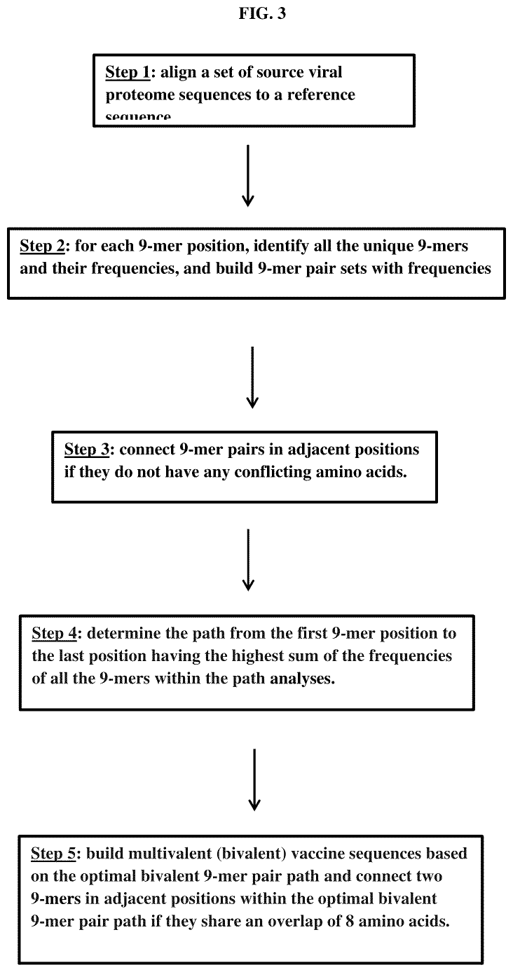

Computationally, the combination of the Conservation Algorithm and the CWA can be described as a positional De Brujin graph based bivalent vaccine sequence design algorithm comprising the following 5 basic steps, illustrated in FIG. 3:

### Step 1: Align a Set of Source Viral Proteome Sequences to a Reference Sequence.

In Step 1, a source population of viral proteome sequences is aligned to a reference sequence. In this example, the reference sequence used was the HIV-1 HXB2, identified by GenBank No. Accession K03455. The amino acid sequences of HXB2 reference polypeptides Env, Gag, Nef and Pol are provided herein as SEQ ID NOs: 403, 404, 405 and 406, respectively. The source population of viral proteome sequences consists of sequences obtained from naturally occurring viruses. Such sequences are publicly available, for example, from the HIV Databases maintained by the Los Alamos National Laboratory, the U.S. Dept. of Health and Human Services, and the National Institutes of Health (hiv.lanl.gov), which was the database used for the source population of sequences in this example. For the purposes of illustration, we focused our analysis on a subset of the viral sequences, here, sequences of Group M Clade B. The alignment was performed using a multiple alignment algorithm, specifically a fast Fourier transform algorithm, MAFFT. Katoh, et al. (2002) Nucleic Acids Res. 30 (14): 3059-66. The base MAFFT software is publicly available and distributed, e.g., under the Berkeley Software Distribution (BSD) license.

Step 2: For Each 9-Mer Position, Pull Out all the Unique 9-Mers and their Frequencies, and Build 9-Mer Pair Sets with Frequencies.

In Step 2, we apply the Conservation Algorithm to the set of aligned sequences. For each sequence in the alignment, starting from the first amino acid of the N-terminus, the algorithm shifts one amino acid position at a time to create a set of all possible amino acid segments that are 9 amino acids in length, referred to as “9-mers.” The algorithm thus creates, for each sequence in the alignment, a set of 9-amino acid subsequences (“9-mers”) starting with the N-terminal amino acid, each subsequence overlapping the preceding subsequence by eight amino acids such that each sequence of length/in the alignment contains (1-8) 9-mers.

Next, for each 9-mer position, the method identifies the two most common unique 9-mers and their prevalence in the aligned set of source viral proteome sequences. Stated another way, starting at position i the two most common unique 9-mers at each position are identified based on their frequency, calculated as the number of times the unique 9-mer occurs at position i in the alignment divided by the total number of sequences in the alignment.

Computationally, each sequence of length l, contains 1-8 9-mers. We define all the 9-mers starting at position i as sᵢⱼ and frequency as fᵢⱼ, j=1,2,3, . . . m. In total there are m unique 9-mers at position i. Each two unique 9-mers (sᵢᵤ, sᵢᵥ) can constitute a 9-mer pair and its frequency is fᵢᵤ+fᵢᵥ. And each 9-mer itself can constitute a 9-mer pair as (sᵢᵤ, sᵢᵤ) and its frequency is fᵢᵤ. Thus, in total, there are m*(m+1)/2 9-mer pairs at each position.

The method then calculates the bivalent conservation for each 9-mer position by summing up the proportions of aligned set of source viral proteome sequences containing either of the two most common 9-mers. To do this, a “bivalent conservation” is calculated for each position by summing the proportion of sequences in the alignment containing either of the two most common unique 9-mers.

Next, a new alignment of conserved regions is created by extracting the sequences in the alignment having a desired bivalent conservation. In this example, we used a bivalent conservation of greater than 80% or greater than 90%, meaning that the two most common 9-mers at position i account for more than 80% or more than 90% of the 9-mers at that position in the new alignment of conserved regions. Stated another way, the method identifies the conserved regions in the new alignment as those in which the sum of the frequencies of the two most common 9-mers at each position is greater than a certain cutoff, e.g., greater than 80% or greater than 90%. Thus, the method also calculates the frequency of each pair of unique 9-mers at each position in the new alignment of conserved regions.

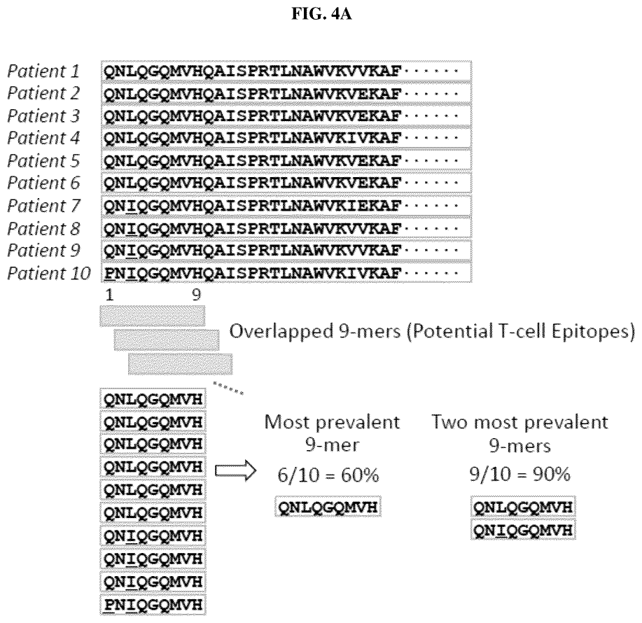

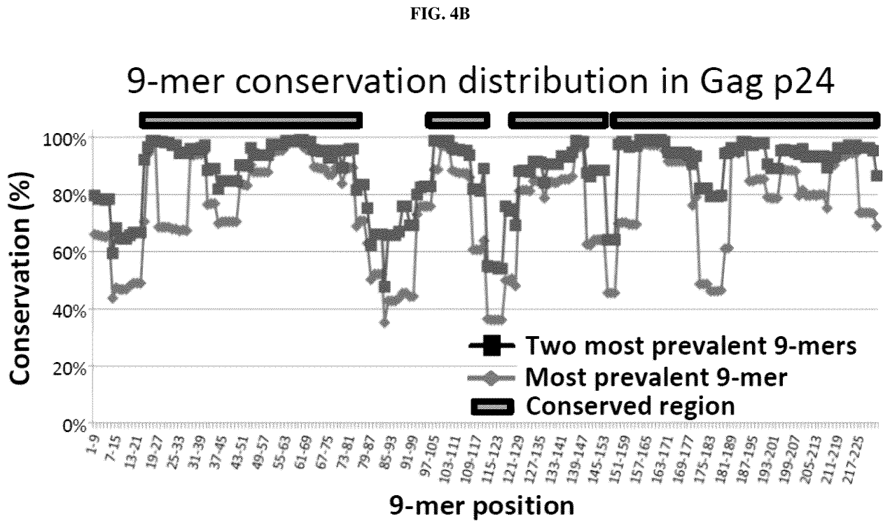

This is illustrated graphically in FIG. 4A. FIG. 4A shows a hypothetical set of 10 input natural sequences, each having a single amino acid variation within the first 9-mer. Across the set of 10 sequences, the 9-mer having an “L” at the third amino acid position occurs 6 times, the 9-mer having an “I” at that location occurs 3 times, and the 9-mer having an “I” at that location but a different amino acid in the first position occurs once. Thus, the Conservation Algorithm selects the two most prevalent 9-mers which together account for 90% of the possible 9-mers at that position in the population of aligned sequences.

Using this analysis, the distribution of highly conserved 9-mers at each position across all of the protein sequences in the population can be determined. This is illustrated graphically in FIG. 4B. The plot shows the conservation distribution for proteins encoded by the Gag gene p24 protein in 9,846 Group M Clade B input sequences obtained from the Los Alamos HIV Sequence database. The y-axis shows bivalent conservation and the x-axis shows the location of the 9-mer relative to the reference sequence, Gag p24 from HXB2. Across the top of the graph the horizontal bars depict conserved regions as those having at least 80% bivalent conservation using the two most prevalent 9-mers at each position. The dark gray line with the squares plots the bivalent conservation at each position using the two most prevalent 9-mers while the light gray line with the diamonds shows conservation using only the most prevalent 9-mer at each position. This analysis demonstrates that the use of the two most prevalent 9-mers improves the identification of structurally conserved sequences with an input population.

We next applied further selection criteria to define the conserved regions, including restricting to regions having greater than 90% bivalent conservation and removing short segments of less than 35 amino acids, e.g., segments 9-35 amino acids in length.

We also included some additional segments from certain regions having at least 80% bivalent conservation and known to be highly immunogenic, in particular, the region of Nef corresponding to amino acids 64-99 of the reference sequence HXB2_K03455 (see, e.g., epitope maps at hiv.lanl.gov/content/immunology/maps/maps.html; Fischer, et al., Nat Med. (2007) 13 (1): 100-6; and Addo, et al., J Virol, (2003) 77 (3): 2081-92).

Step 3: Connect 9-mer Pairs in Adjacent Positions if They Do Not Have Any Conflicting Amino Acids.

Using this modified set of conserved regions, we applied the CWA to build bivalent sequence constructs. The CWA connects 9-mer pairs in adjacent positions of the alignment of conserved regions that share an overlap of eight amino acids.

Computationally, each 9-mer s contains 9 amino acids, we write s[x:y] to represent the amino acid subsequence from position x to y, y−x+1 amino acids in total:

$${s}_{iu} [ 2 : 9 ] == {s}_{i + Ip} [ 1 : 8 ] and {s}_{iv} [ 2 : 9 ] == {s}_{i + Iq} [ 1 : 8 ] or$$

$${s}_{iu} [ 2 : 9 ] == {s}_{i + Iq} [ 1 : 8 ] and {s}_{iv} [ 2 : 9 ] == {s}_{i + Ip} [ 1 : 8 ] .$$

Step 4: Find the Optimal Path from the First 9-Mer Position to the Last Position in Terms of the Sum of the Frequencies of all the 9-Mers within the Path.

In Step 4, the algorithm builds a directed acyclic graph in which each 9-mer pair is a node and the edges between adjacent nodes are formed from the connected 9-mer pairs in the adjacent positions with the weight of each edge equal to the frequency of the downstream 9-mer pair. This directed acyclic graph is a positional De Brujin graph. Such graphs have been described in connection with assemblies of next generation sequencing data, for example as described in Ronen et al., Bioinformatics (2012) 28:188-196.

In the present example, we add a source node and connect it with all of the nodes in the first position; and we add a sink node and connect it with all of the nodes in the last position. In a directed graph, a source node is a node that only has out flow and a sink node is a node that only has in flow. Here, the source node is a dummy node that connects to all the 9-mer pair nodes in the first position, and the sink node is a dummy node that connects to all the 9-mer pair nodes in the last position.

We then negate all of the weights and find the optimal path from the source node to the sink node, where the optimal path is defined in terms of the sum of the frequencies of all 9-mer pairs. The task of finding the optimal path is performed, for example, using the Bellman-Ford algorithm. Generally, the Bellman-Ford algorithm computes the shortest paths from a single source vertex to all of the other vertices in a weighted directed graph. A directed graph is one made up of a set of vertices connected by edges, where the edges have a direction associated with them.

Computationally, the basic idea is to model the maximum coverage bivalent vaccine construction problem as a classic graph theory problem where the solution is finding the minimum path in a directed acyclic graph. The computational steps can be summarized as follows:
(4-1) Treat each 9-mer pair as a node, and build edges between adjacent nodes in Step 3;
(4-2) Adding a source node and connect it with all the nodes at the 1st position;
(4-3) Adding a sink node and connect it with all the nodes at the last position;
(4-4) Weight of each edge equals to the frequency of downstream 9-mer pair; and
(4-5) Negating all the weights and finding the optimal path using the Bellman-Ford algorithm.
   Step 5: Build Bivalent Vaccine Sequences Based on the Optimal Bivalent 9-Mer Pair Path and Connect Two 9-Mers in Adjacent Positions within the Optimal Bivalent 9-Mer Pair Path if they Share an Overlap of 8 Amino Acids.

In Step 5, a bivalent construct is built by connecting two 9-mers in adjacent positions within the optimal bivalent 9-mer path if they share an overlap of eight amino acids, thereby creating two sequences of connected 9-mers which together form the bivalent construct. The connected adjacent 9-mer pairs all have an 8 amino acid overlap, so they will be assembled into two sequences. For example, one 9-mer pair (AIIIIIIIS (SEQ ID NO: 464), MIIIIIIII (SEQ ID NO: 465)) can be connected with another 9-mer pair (IIIIIIISK (SEQ ID NO: 466), IIIIIIIIR (SEQ ID NO: 467)) and make two sequences (bivalent sequences): AIIIIIIISK (SEQ ID NO: 468) and MIIIIIIIIR (SEQ ID NO: 469).

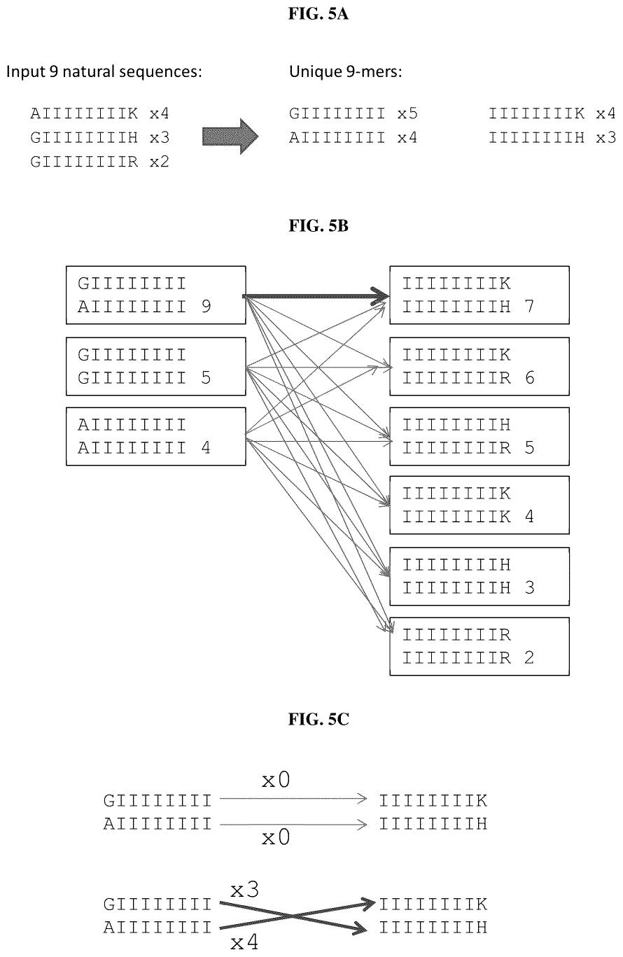

This method is illustrated graphically in FIGS. 5A-5C shows a hypothetical set of 9 source viral sequences having, at the first position, 2 unique 9-mers and at the second adjacent position 3 unique 9-mers. The frequency of each sequence is indicated to the right of the sequence as ‘times’, e.g., “x5” means that sequence occurs 5 times in the source set. FIG. 5B depicts the building of the positional De Brujin graph in which each node is one bivalent 9-mer pair. Where two bivalent 9-mer pairs in adjacent positions share an overlap of eight amino acids they are connected to build an edge. In this manner the directed acyclic graph is created. FIG. 5C illustrates the finding of the optimal path. As noted above, the optimal path is defined in terms of the sum of the frequencies of all 9-mer pairs. This is accomplished by finding the connection between adjacent 9-mers that provides the highest conservation with reference to the input sequences. Thus, in FIG. 5C, connecting the two 9-mer pairs as shown in the top set of four pairs provides the following bivalent sequences,

(SEQ ID NO: 471)
GIIIIIIIIK x0
(SEQ ID NO: 472)
AIIIIIIIIH x0.

Neither of these sequences is present in the source sequences shown in FIG. 5A.

In contrast, connecting the two 9-mer pairs as shown in the bottom set of four pairs in FIG. 5C provides the following bivalent sequences,

(SEQ ID NO: 473)
GIIIIIIIIH x3
(SEQ ID NO: 474)
AIIIIIIIIK x4.

Each of these is present, 3 or 4 times, respectively, in the source sequences shown in FIG. 5A. Accordingly, it is these second pair of bivalent sequences that is selected by the algorithm because it maximizes conservation relative to the source sequences.

Computationally, this can be illustrated by the following exemplary cases:
Case 1: if siu[2:9]=si+1p[1:8] and siv[2:9]=si+1q[1:8], connect siu with si+1p and siv with si+1q;
Case 2: if siu[2:9]=si+1q[1:8] and siv[2:9]=si+1p[1:8], connect siu with si+1q and siv with si+1p;
Case 3: if siu[2:9]=si+1p[1:8] and siv[2:9]=si+1q[1:8] and siu[2:9]=si+1q[1:8] and siv[2:9]=si+1p [1:8], the selection of connection is based on the prevalence of the two connections in natural sequences:

Denote the prevalence of the co-existence of six and si+1y in input sequences as Cixy;
If Ciup+Civq>Ciuq+Civp, connect siu with si+1p and siv with si+1q;
If Ciuq+Civp>Ciup+Civq, connect siu with si+1q and siv with si+1p;
If Ciup+Civq=Ciuq+Civp, backtrack and combine the prevalence of the co-existence of 9-mer pairs in positions i-1 and i until the first position. If there is no difference between two different connections, randomly pick one.

This backtrack and co-existence prevalence approach considers prevalence of peptides longer than 9 amino acids and further differentiates the present algorithm from other graph-based methods.

Next, constructed sequences from regions not adjacent to one another in the natural sequence, that is, regions which could not be joined according to the CWA as described above due to their lacking an 8 amino acid overlap, were combined using one of three different linker strategies: 1. direct fusion without any linker; 2. insert ‘AAA’ linker between each two conserved regions; 3. direct fusion without any linker for segments within the same protein and insertion of an F2A linker between segments from different proteins.

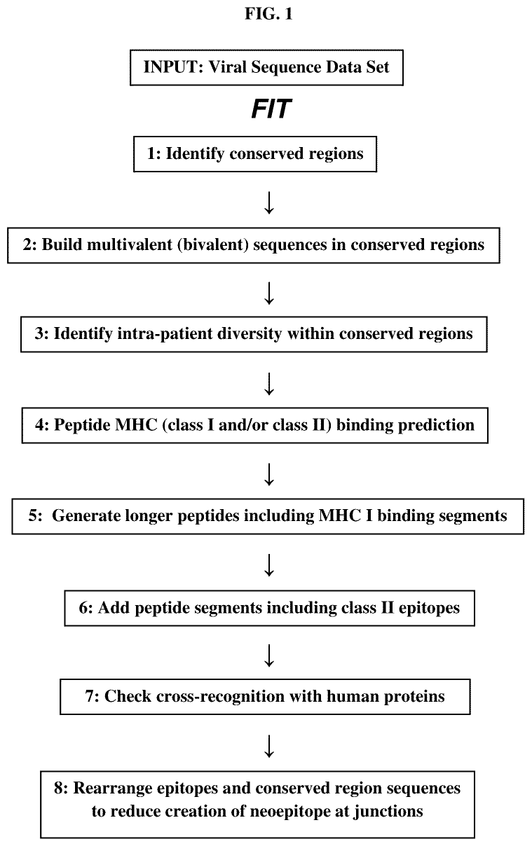

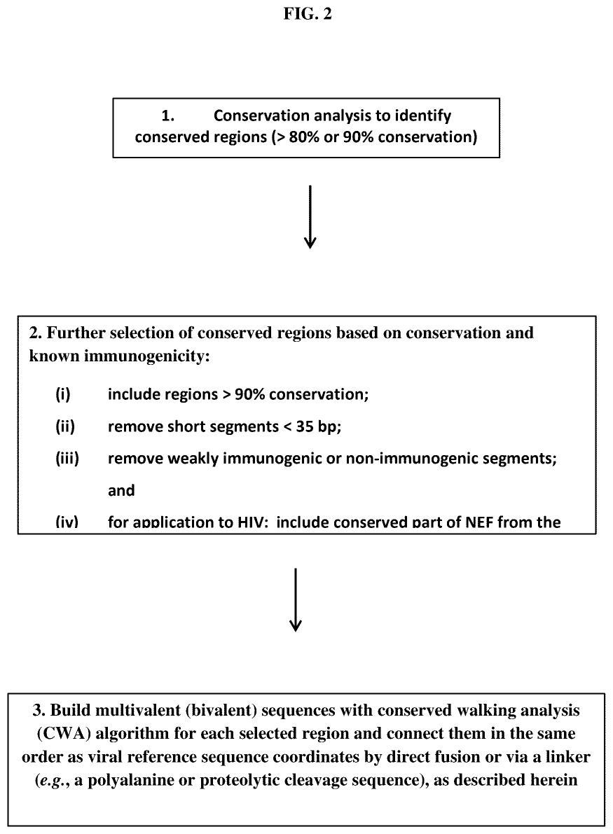

An overview of the Conserved Walking Analysis (CWA) method is shown in FIGS. 1-2. The fusion polypeptides of SEQ ID NOs: 345-350 and the sequences in Table 2, which have polypeptide segments encoding by the HIV-1 Gag, Nef and Pol genes, are exemplary immunogenic fusion polypeptide sequences designed according to this method.

### Example 2

### Illustrated Implementation of the Conservation Analysis and Conserved Walking Analysis (CWA) Applied to Proteins Encoded by HIV-1 Genes

This example describes a similar implementation based on conserved HIV-1 regions of (i) Gag and Nef (“GagNef”), (ii) Pol, or (iii) Pol and Env (“PolEnv”).

In Example 1 above, the Conservation algorithm was applied to identify a set of all candidate conserved regions in the protein coding regions of the target genes Gag, Nef, Env and Pol. In this example, we utilized the protein coding regions of (1) Gag and Nef, (2) Pol or (3) Pol and Env to generate three different bivalent constructs, “GagNef,” “Pol” and “PolEnv,” respectively. As in Steps 1-2 of Example 1 above, we first aligned the source sequences and then applied the Conservation Algorithm to identify a set of all candidate conserved regions in the protein coding regions of the target genes, which were either Gag and Nef, Pol, or Pol and Env. As above, we then we applied further selection criteria based on conservation and known immunogenicity (see, e.g., epitope maps at hiv.lanl.gov/content/immunology/maps/maps.html and Fischer, et al., Nat Med. (2007) 13 (1): 100-6). In certain sequences including polypeptide segments encoding by the Pol gene, we excluded sequence segments including one or both of the “YMDD” motif (SEQ ID NO: 462) in reverse transcriptase and the “DTG” motif in protease, because they may affect expression the maintenance of enzymatic activity.

Using this modified set of conserved regions, we applied the CWA to build bivalent sequence constructs, as in Steps 3-5 in Example 1.

Some polypeptide segments were connected by a polyalanine linker (e.g., AA, AAA or AAY), chosen for demonstration purposes because it is a small flexible linker that is unlikely to have a significant influence on protein structure. If we determined that it was possible to fuse polypeptide segments without creating a deleterious or undesirable junctional epitope, e.g., such as one that may stimulate T cells that may cross react to self-antigens, a fusion approach was used. If we determined that a deleterious or undesirable junctional epitope may be created, a flexible linker was inserted between polypeptide segments.

For this Example, we applied a further analysis of the junctional regions for possible presentation of deleterious epitopes and arranged the segments to reduce or avoid the creation of such junctional epitopes.

Different arrangements of peptide segments generate different junction 9-mers that can induce different junction responses. We developed a polypeptide segment arrangement tool to examine MHC binding affinities and cross-recognition with human peptides for all the junction 9-mers in each arrangement. Our internally developed polypeptide segment arrangement tool searches different arrangements of peptides and determines the best arrangement with minimal junction response based on in silico prediction results of applying the two analyses described below ((1) in-silico HLA binding analysis and (2) human proteome analysis to identify epitopes that may prime T cells that may recognize self-antigens) on the junctions of 9-mers. The junctional response score between each two adjacent segments is determined by the sum of the number of junction 9-mers that are predicted to have high binding affinities to target HLA alleles and the number of human proteins predicted to have peptides or T cell recognition motifs with any junction 9-mers. The score of each segment arrangement is determined by the sum of the junctional response scores for all the junctional regions in each segment arrangement.
1) When there are less than 15 peptide segments, our internally developed polypeptide segment arrangement tool searches all the possible arrangements and determines the best one with minimal junction response (the lowest segment arrangement score)
2) When there are at least 15 peptide segments, our internally developed polypeptide segment arrangement tool uses a ‘greedy’ strategy. It first creates all the junctions and then starts from the best junction in terms of predicted junctional response. Next, it searches for the next compatible best junction iteratively and assembles all the peptide segments.

In-silico MHC class I (human HLA) binding analysis: Antigen processing, presentation, and T cell receptor recognition are complex processes that remain incompletely understood. Intracellular and extracellular antigens are processed within endosomal compartments, and the cytoplasm by the proteasome and trafficked to endosomal compartments such as the ER where they peptide fragments interact with MHC molecules. Stable peptide-MHC complexes are trafficked to the cell surface where they can be recognized by a T cell expressing a TCR with the appropriate specificity. One of the most selective steps in antigen processing and presentation is HLA binding. HLA binding affinities can be predicted using various tools such as NetMHC or MHCflurry, or large internal datasets derived from immunopeptidome analyses and confirmed by experimental binding data as well as epitopes defined from patient samples. These tools are publicly available and are described, for example, in Lundegarrd et al., Nucleic Acids Res. 2008 Jul. 1; 36 (Web Server issue): W509-12 and O'Donnell, et al., Cell Systems 2018 7:129-132. In this example we used NetMHC. The default settings were used for all the parameters in NetMHC, along with inputting information for peptide sequences and HLA alleles. Predicted binding affinities with an IC50 value less than 1,000 nM are considered as low binding affinities.

Human proteome cross-recognition analysis: Epitopes similar to human peptides may induce tolerogenic responses or responses that may cross-react with self-antigens. We searched all the 9-mers in our vaccine against public human protein databases (e.g., Uniprot, NCBI). If an HIV peptide 9-mer has at least a 5-residue amino acid sequence identity with a human peptide 9-mer, and both are predicted to have high binding affinities to the same alleles, they are considered as cross-conserved 9-mers. We downloaded all the human protein sequences from the UniProt database and built a tool to support efficient search of a given 9-mer against all the human protein 9-mers with up to 4 mismatches (at least 5 matches).

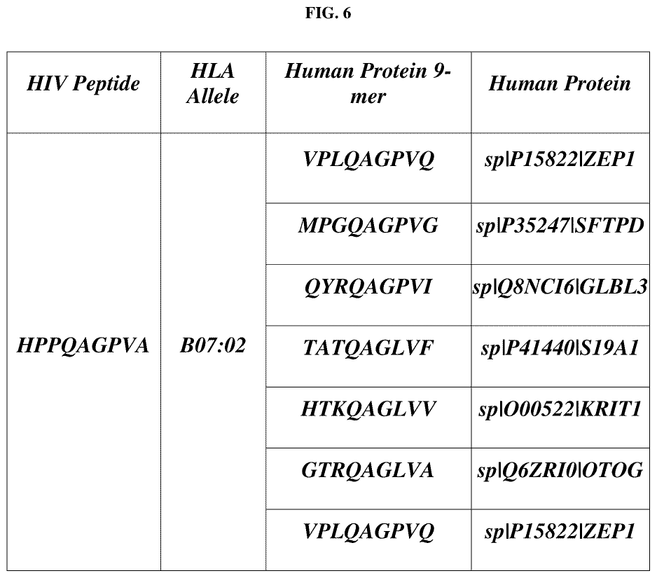

FIG. 6 illustrates the results of human proteome cross-recognition analysis. In this example, we searched HIV-1 peptide 9-mers over human protein databases and identified all the human protein 9-mers sharing a certain number of amino acids (at least 5 tentatively) and are predicted to have high binding affinities (e.g., IC50 of less than about 1000 nM or having a percentile rank within the top 5% in a population of polypeptide segments) to the same alleles based on the in silico MHC class I analysis described herein. Such HIV 9-mers having both high sequence identity (e.g., having have at least 55% (5 of 9 amino acid residues), e.g., at least 65% (6 of 9 amino acid residues), e.g., at least 75% (7 of 9 amino acid residues), e.g., at least 85% (8 of 9 amino acid residues)) to a peptide segment of a human protein and high predicted MHC class I binding affinity are excluded because they may induce tolerogenic responses or responses that may cross-react with human self-antigens (defined herein as “deleterious epitopes.”).

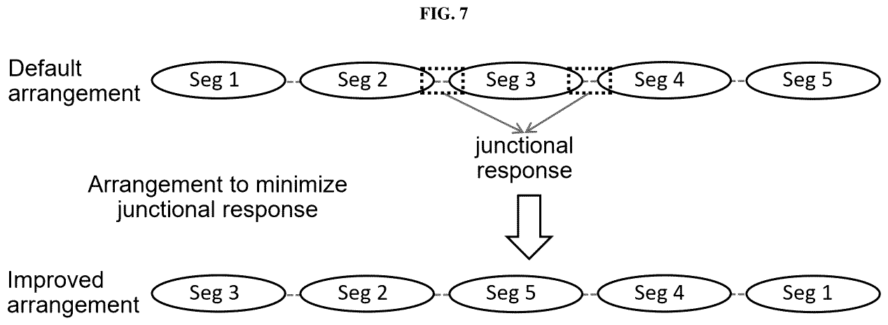

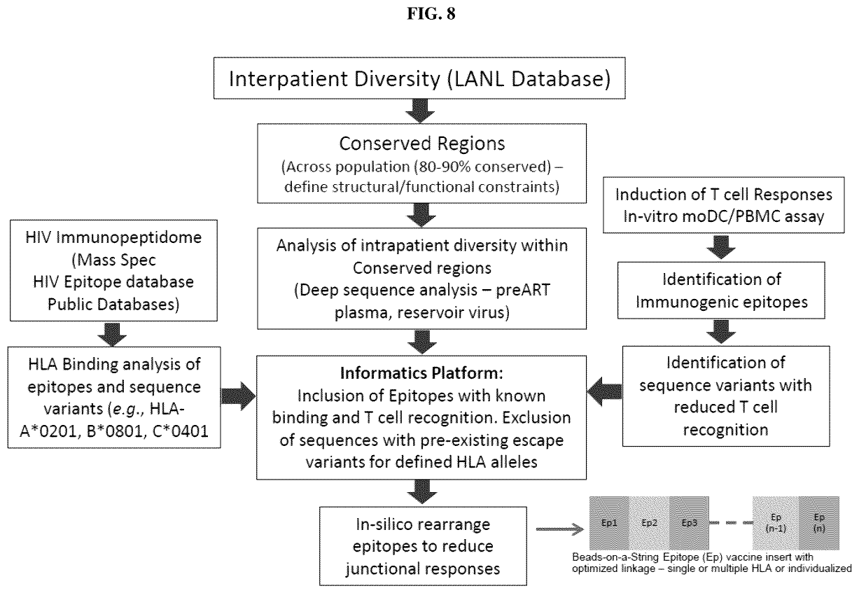

FIG. 7 illustrates how polypeptide segment arrangement analysis can reduce or eliminate possible presentation of deleterious or undesirable epitopes in junction regions. In the illustrated default arrangement, the junction 9-mers between Seg 2 and Seg3, and between Seg 3 and Seg 4 are predicted to produce junctional sequences that may induce tolerogenic or self-reactive responses in a human (e.g., having either high MHC binding affinity based on in silico HLA binding analysis or cross-recognition with human proteins based on human proteome cross-recognition analysis). We applied an algorithm that searches different arrangements and determines an arrangement that results in reduced or eliminated predicted junctional sequences that may induce tolerogenic or self-reactive responses in a human.

The fusion polypeptides of SEQ ID NOs: 351-366 and 407-410 are exemplary immunogenic fusion polypeptide sequences designed according to this method.

### Example 3

### Immunogenicity of ChAd and SAM Vaccines Encoding HIV Immunogens in Mice

This study investigated heterologous HIV-1 prime/boost vaccine system to induce robust T cell responses against HIV antigens. The prime/boost vaccine comprises a chimpanzee adenovirus (ChAd) prime (ChAd-01 or ChAd-02) and a self-amplifying RNA (SAM) boost (SAM-01 or SAM-02); these vectors are known to elicit significant T-cell responses, particularly CD8+ cytotoxic T cells, against disease-specific targets engineered into the vaccine.

This study evaluated the immunogenicity of ChAd and SAM vaccines encoding HIV immunogens 1A and 1B in Balb/c mice. Four vaccines were evaluated, ChAd-01, ChAd-02, SAM-02, and SAM-02 (corresponding to SEQ ID NOs: 520-523, respectively). All vaccines induced potent T cell responses that were specific to the encoded antigens in mice.

Each HIV Immunogen covers 12 conserved regions across HIV-1 Clade B Group-specific antigen (Gag), Polymerase (Pol), and accessory protein Negative Regulatory Factor (Nef). The conserved regions are polypeptide regions (typically >15 amino acids) within these viral proteins and are concatenated to make a single immunogen.

Two different immunogen sequences were designed (Immunogens 1A and 1B, corresponding to SEQ ID NOs: 524-525 (nucleotide sequence) or SEQ ID NOs: 527-528 (amino acid sequence), respectively) for use in a bivalent vaccine. Immunogens 1A and 1B are identical in structure (i.e., containing the same conserved regions), with different amino acids at certain positions introduced to cover more than 80% of potential human leukocyte antigen (HLA) class I epitopes among circulating clade B viruses.

The objective of this study was to evaluate the immunogenicity of these vaccines in vivo in Balb/c mice.

### Materials and Methods

### Vaccines

The vaccines used in this study encode HIV immunogen 1A and 1B, corresponding to SEQ ID NOs: 527 and 528, respectively. The ChAd68 vector is an E1, E3, and E4Orf2-4 deleted replication incompetent chimpanzee adenovirus vector. The self-amplifying RNA vector (SAM) is based on the Venezuelan Equine Encephalitis virus (VEE) in which the structural genes have been replaced with a cassette expressing Immunogens 1A or 1B from the subgenomic (26S) promoter. The SAM RNA is then packaged in lipid nanoparticles (LNP) for delivery. Vaccines were evaluated in two separate studies, see Table 2 for details.

TABLE 2
Vaccines

| Vaccine | | | | | |
|---|---|---|---|---|---|
| Name | SEQ ID NO: | Vector | Immunogen | Dose | |
| ChAd-01 | SEQ ID NO: 520 | ChAd | 1A | 5 × 10¹⁰ | VP |
| SAM-01 | SEQ ID NO: 522 | SAM | 1A | 10 | μg |
| ChAd-02 | SEQ ID NO: 521 | ChAd | 1B | 5 × 10¹⁰ | VP |
| SAM-02 | SEQ ID NO: 523 | SAM | 1B | 10 | μg |

### Mouse Immunizations

All mouse studies were conducted at Murigenics under IACUC approved protocols. Female Balb/c mice (Envigo), aged 6-8 weeks old were used. ChAd vaccines were stored at-80° C. and thawed at 37° C. on the day of immunization. SAM vaccines were stored at −80° C. Prior to immunization, SAM vaccines were thawed at room temperature, diluted to 0.1 mg/mL with PBS and filtered with an 0.2 micron PES filter. Vaccines were delivered intramuscularly to the tibialis anterior, as two bilateral injections of 50 μL per leg.

### Splenocyte Isolation

Mouse spleens were extracted 13-14 days following immunization. Spleens were resuspended in RPMI complete (RPMI+10% FBS) and dissociated using the gentleMACS Dissociator (Milltenyi Biotec). Dissociated cells were filtered using a 40 μm strainer and red blood cells were lysed with ACK lysing buffer (150 mM NH₄Cl, 10 mM KHCO₃, 0.1 mM EDTA). Following lysis, cells were filtered with a 30 μm strainer and resuspended in RPMI complete.

### IFNγ ELISpot

IFNγ ELISpot assays were performed using pre-coated 96-well plates (Mabtech, Mouse IFNγ ELISPOT PLUS, ALP) following manufacturer's protocol. Splenocytes were plated at 1×10⁵ cells/well in triplicate, stimulated with four overlapping peptide pools spanning the immunogen sequence (provided by Gilead) at final concentration of 2 μg/mL per peptide and incubated overnight at 37° C. in RPMI+10% FBS. The peptide pools spanned the Pol (pool 1, 316 amino acids, 146 peptides; pool 2, 320 amino acids, 142 peptides), Gag (274 amino acids, 129 peptides) and Nef (66 amino acids, 26 peptides) antigens. DMSO was used as a negative control for each sample. Plates were washed with PBS and then incubated with anti-mouse IFNγ mAb biotin (Mabtech) for two hours, followed by an additional wash and incubation with Streptavidin-ALP (Mabtech) for one hour. After final wash, plates were incubated for ten minutes with BCIP/NBT (Mabtech) to develop the immunospots. Wells were imaged and spots enumerated using AID reader (Autoimmun Diagnostika). Spot values were adjusted based on the well saturation according to the formula: AdjustedSpots=RawSpots+2*(RawSpots*Saturation/(100-Saturation)).

Samples with well saturation too high to be counted accurately were considered too numerous to count (TNTC) and set to the maximum adjusted value. Each sample was background corrected by subtracting the average value of the negative control wells. Data was normalized to spot forming units (SFU) per 1×10⁶ splenocytes by multiplying the corrected spot number by 1×10⁶/cell number plated. Data processing was performed using the R programming language.

### Methods and Results

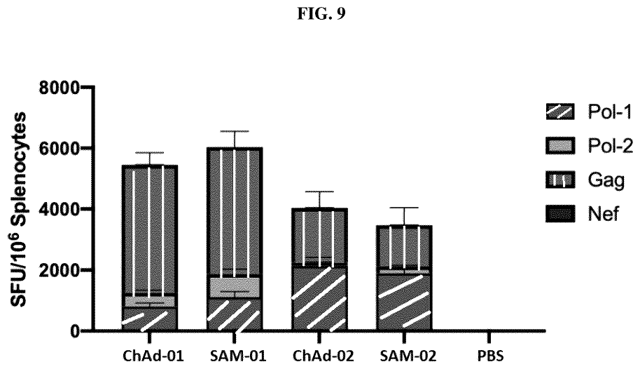

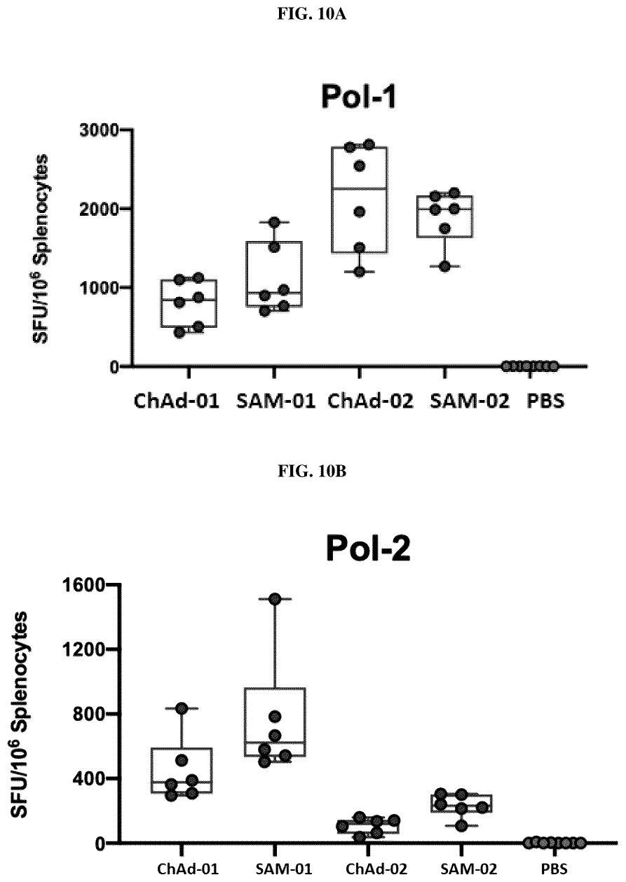

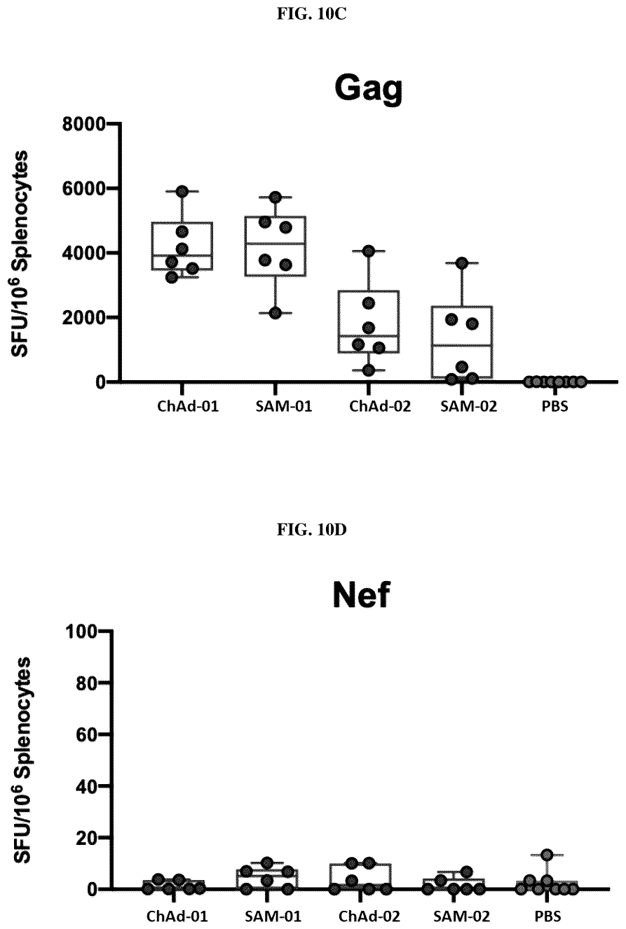

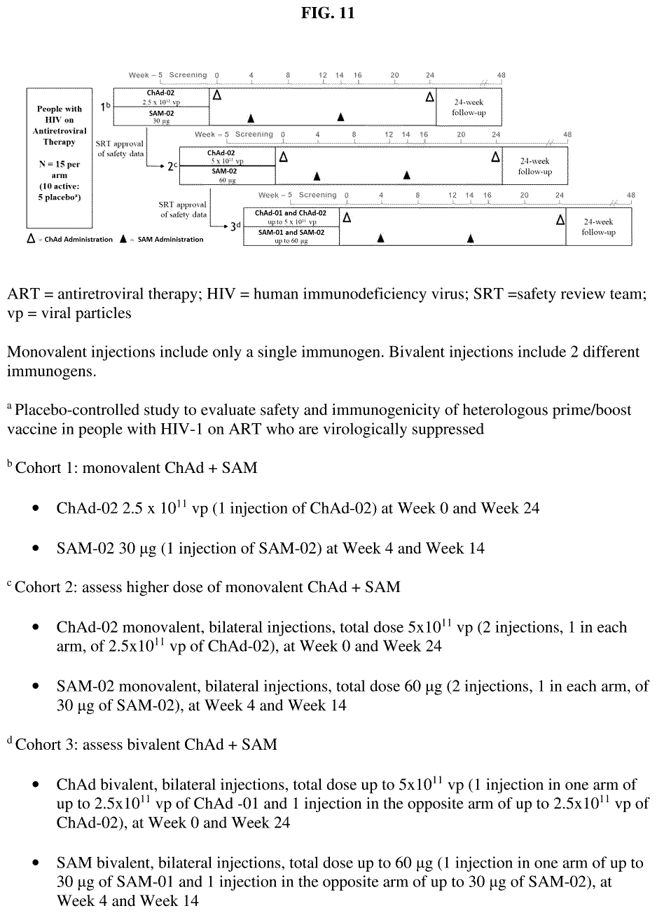

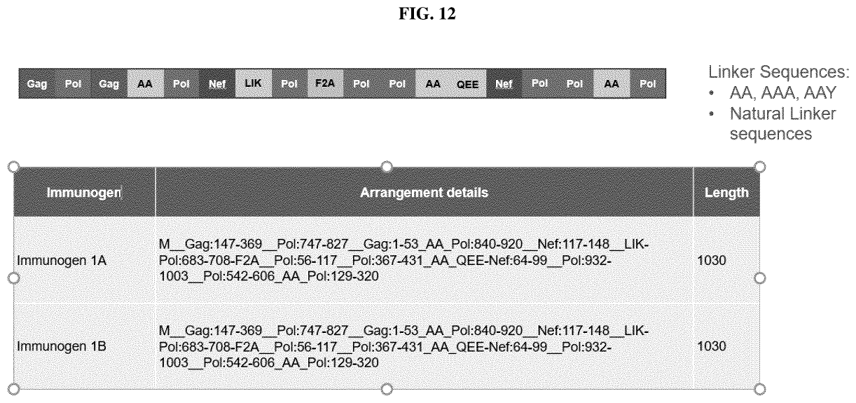

Balb/c mice were immunized with 5×10¹⁰ VP of SEQ ID NO: 520 and SEQ ID NO: 521, 10 μg of SEQ ID NO: 522 and SEQ ID NO: 523 (n=6 per group), or PBS as a negative control (n=4). Antigen-specific T cell responses were assessed at 13-14 days post immunization by IFNγ ELISpot. Strong T cell responses were detected in all vaccinated mice (FIG. 9, FIGS. 10A-D, Table 6, Table 7). No antigen-specific response was detected in control mice. Positive responses were detected in all vaccinated mice to both Pol and Gag peptide pools, compared to PBS injected control mice, while no response to the Nef pool was detected in any mice (FIGS. 10A-D, Table 7). Similar total antigen-specific T cell responses were observed with both ChAd and SAM vaccine vectors. Overall T cell responses to the Pol-2 and Gag peptide pools were slightly higher with Immunogen 1A vaccines compared to 1B, while responses to Pol-1 peptide pool were higher with Immunogen 1B vaccines compared to 1A. This may be a result of differential epitope expression as a result of sequence differences between the immunogens or experimental variability, as the 1A and 1B expressing vaccines were evaluated in two separate studies.

TABLE 6
Antigen-specific T cell response (SFU/10⁶ splenocytes) 2-weeks post vaccination

| | Pol-1 | | Pol-2 | | Gag | | Nef | | Total | |
|---|---|---|---|---|---|---|---|---|---|---|
| Vaccine | Mean | SE | Mean | SE | Mean | SE | Mean | SE | Mean | SE |
| ChAd-01 | 808 | 119 | 451 | 83 | 4194 | 396 | 1 | 1 | 5453 | 435 |
| SAM-01 | 1114 | 185 | 764 | 155 | 4162 | 517 | 5 | 2 | 6044 | 634 |
| ChAd-02 | 2134 | 279 | 107 | 20 | 1794 | 534 | 4 | 2 | 4039 | 617 |
| SAM-02 | 1893 | 141 | 232 | 29 | 1346 | 575 | 2 | 1 | 3473 | 556 |
| PBS | 1 | 1 | 1 | 1 | 0 | 0 | 1 | 1 | 3 | 1 |

TABLE 7
Antigen-specific T cell response (SFU/10⁶ splenocytes) for
each animal 2-weeks post vaccination

| Vaccine | Animal | Pol-1 | Pol-2 | Gag | Nef | Sum |
|---|---|---|---|---|---|---|
| ChAd-01 (SEQ ID | 1 | 1123 | 834 | 4658 | 0 | 6614 |
| NO: 520) | 2 | 507 | 513 | 5900 | 0 | 6920 |
| | 3 | 1100 | 389 | 3246 | 4 | 4738 |
| | 4 | 432 | 364 | 3519 | 0 | 4315 |
| | 5 | 876 | 296 | 3718 | 4 | 4893 |
| | 6 | 811 | 309 | 4121 | 0 | 5242 |
| SAM-01 (SEQ ID | 1 | 768 | 1512 | 5719 | 0 | 7999 |
| NO: 522) | 2 | 703 | 503 | 3769 | 7 | 4982 |
| | 3 | 1514 | 665 | 4950 | 10 | 7140 |
| | 4 | 970 | 542 | 2130 | 7 | 3650 |
| | 5 | 1827 | 579 | 3621 | 3 | 6030 |
| | 6 | 899 | 783 | 4782 | 0 | 6464 |
| ChAd-02 (SEQ ID | 1 | 1960 | 159 | 1057 | 10 | 3186 |
| NO: 521) | 2 | 2544 | 135 | 363 | 0 | 3042 |
| | 3 | 1200 | 37 | 1676 | 3 | 2916 |
| | 4 | 2780 | 104 | 4058 | 0 | 6941 |
| | 5 | 2814 | 64 | 1166 | 0 | 4043 |
| | 6 | 1507 | 141 | 2445 | 10 | 4102 |
| SAM-02 (SEQ ID | 1 | 1989 | 108 | 3685 | 7 | 5788 |
| NO: 523) | 2 | 2199 | 214 | 85 | 0 | 2498 |
| | 3 | 1748 | 221 | 109 | 0 | 2078 |
| | 4 | 2157 | 240 | 1802 | 0 | 4200 |
| | 5 | 1999 | 301 | 463 | 3 | 2766 |
| | 6 | 1268 | 305 | 1934 | 0 | 3507 |
| PBS | 1 | 0 | 0 | 0 | 0 | 0 |
| | 2 | 3 | 0 | 0 | 0 | 3 |
| | 3 | 0 | 0 | 0 | 0 | 0 |
| | 4 | 0 | 3 | 0 | 3 | 7 |
| | 5 | 3 | 7 | 3 | 0 | 13 |
| | 6 | 3 | 0 | 10 | 13 | 27 |
| | 7 | 0 | 0 | 3 | 0 | 3 |
| | 8 | 0 | 0 | 0 | 3 | 3 |

### Conclusions

This study evaluated the immunogenicity of ChAd and SAM vaccines encoding HIV immunogens 1A and 1B in Balb/c mice. Potent T cell responses to the encoded antigens were observed in all vaccinated mice. T cell responses to Pol and Gag immunogens were detected in all vaccinated mice, while no responses to the Nef antigen were detected. The Nef immunogen sequences encoded by the vaccine were selected for maximum coverage of human HLA epitopes, which are not predicted to be immunogenic in mice. In addition, the region of Nef encoded by the vaccines represents only 66 amino acids, and constitutes a very small portion of the protein, both of which are likely reasons for the lack of response to the Nef antigen.

In summary, ChAd-01, ChAd-02, SAM-01, and SAM-02 induced potent T cell responses to encoded HIV immunogens in all vaccinated mice.

### Example 4

A Phase 1b Study to Evaluate the Safety and Tolerability of HIV Vaccine Regimens in People with HIV-1 on Antiretroviral Therapy who are Virologically Suppressed

Study design: This is a Phase 1b, randomized, multicenter, single-blind, placebo-controlled study to evaluate the safety, tolerability, and immunogenicity of HIV vaccine regimens comprising an adenovirus vector and a self-amplifying mRNA (SAM) (e.g., ChAd-01 and SAM-01 (corresponding to SEQ ID NOs: 520 and 522, respectively) or ChAd-02 and SAM-02 (corresponding to SEQ ID NOs: 521 and 523, respectively) in people with HIV (−1) (PWH) on antiretroviral therapy (ART) who are virologically suppressed. The study includes 3 cohorts randomized in a 2:1 ratio to receive (a) ChAd-01 and SAM-01, (b) ChAd-02 and SAM-02, or (c) placebo. Participants maintain ART treatment for the duration of the study.

For each participant, the study comprises a screening period (up to 5 weeks), treatment period (24 weeks), and follow-up period (24 weeks).

Sentinel Dosing: Each cohort is conducted with a sentinel group consisting of 3 participants (randomized 2:1 active to placebo). Dosing beyond the sentinel group is performed following completion of safety assessments through at least Day 8. Progression between cohorts is governed by review of safety assessments through at least Day 36 from prior cohorts and application of stopping rules.

Participants are monitored onsite for 30 minutes after inoculation. If participants experience new or worsening symptoms, postdose safety monitoring may be extended at the investigator's discretion in consultation with the medical monitor.

This study includes 3 cohorts of approximately 15 participants per cohort who are randomized in a 2:1 ratio to receive either ChAd+SAM or placebo. Participants maintain ART treatment for the duration of the study. A schematic diagram of the design, procedures, and treatments is provided in FIG. 11 and Table 9. Details on study drugs and dose levels by cohort are presented in Table 8.

TABLE 8
Study Drugs and Dose Levels

| | | Week 0 | Week 4 | Week 14 | Week 24 |
|---|---|---|---|---|---|
| Participants | | Day 1 | Day 29 | Day 99 | Day 169 |
| Cohort 1 | n = 10 | ChAd-02 prime | SAM-02 boost | SAM-02 boost | ChAd-02 |
| | | (1 IM | (1 IM injection), | (1 IM injection), | prime |
| | | injection), | 30 μg | 30 μg | (1 IM injection), |
| | | 2.5 × 10¹¹ vp | | | 2.5 × 10¹¹ vp |
| | n = 5 | Placebo | Placebo | Placebo | Placebo |
| | | (IM injection) | (IM injection) | (IM injection) | (IM injection) |
| Cohort 2 | n = 10 | ChAd-02 prime | SAM-02 boost | SAM-02 boost | ChAd-02 prime |
| | | (2 IM injections), | (2 IM injections), | (2 IM injections), | (2 IM injections), |
| | | 5 × 10¹¹ vp total | 60 μg total | 60 μg total | 5 × 10¹¹ vp total |
| | n = 5 | Placebo | Placebo | Placebo | Placebo |
| | | (IM injection) | (IM injection) | (IM injection) | (IM injection) |
| Cohort 3 | n = 10 | ChAd prime | SAM boost | SAM boost | ChAd prime |
| | | (1 IM injection | (1 IM injection of | (1 IM injection of | (1 IM injection |
| | | ChAd-01 and 1 | SAM-01 and 1 IM | SAM-01 and 1 | ChAd-01 and 1 |
| | | IM injection of | injection of SAM- | IM injection of | IM injection of |
| | | ChAd-02), | 02), up to 60 μg | SAM-02), up to | ChAd-02), |
| | | up to 5 × 10¹¹ vp | total | 60 μg total | up to 5 × 10¹¹ vp |
| | | total | | | total |
| | n = 5 | Placebo | Placebo | Placebo | Placebo |
| | | (IM injection) | (IM injection) | (IM injection) | (IM injection) |

For each participant, the study comprises:
Screening period (up to 5 weeks)
Eligible participants should return to the study center for baseline/Day 1 assessments within 5 weeks of the screening visit.
Treatment period (24 weeks)
   Participants have approximately up to 15 visits during the treatment period (Table 9). All study visits are to be completed within ±2 days, unless otherwise specified.
Follow-up period (24 weeks)
   Participants have approximately 5 visits during the follow-up period (Table 9). All study visits are to be completed within ±3 days, unless otherwise specified. An end of study visit is performed at Day 337 (Week 48).

Number of Participants Planned: Approximately 45 participants (approximately 15 participants per cohort).

Target Population: The study is conducted in adults with HIV-1 who have been virologically suppressed (HIV-1 RNA <50 copies/mL) on ART for at least 12 consecutive months prior to screening.

Duration of Treatment: Participants receive 4 doses of study drugs over a maximum of 24 (±1) weeks.

TABLE 9
Study Procedures Table

| | | Treatment Period | | | | | | | | | | | | | | | Follow-up Period | | | | | | |
|---|---|---|---|---|---|---|---|---|---|---|---|---|---|---|---|---|---|---|---|---|---|---|---|
| | | Week | | | | | | | | | | | | | | | | | | | | | |
| | | 0 | | | 2 | 4 | | | 6 | 8 | 14 | | 16 | 18 | 24 | | 26 | 28 | 36 | 44 | 48 | | |
| | | Day | | | | | | | | | | | | | | | | | | | | | |
| | | 1 | 2 | 3 | 15 | 29 | 30 | 31 | 43 | 57 | 99 | 100 | 113 | 127 | 169 | 170 | 183 | 197 | 253 | 309 | 337 | | |
| | SCR | Visit Window (days) | | | | | | | | | | | | | | | | | | | | | |
| | −35 to −1 | ±2 | ±2 | ±2 | ±2 | ±2 | ±2 | ±2 | ±2 | ±2 | ±2 | ±2 | ±2 | ±2 | ±2 | ±2 | ±3 | ±3 | ±3 | ±3 | ±3 | ESDD^{a} | ET |
| Written informed | X | | | | | | | | | | | | | | | | | | | | | | |
| consent | | | | | | | | | | | | | | | | | | | | | | | |
| Study contraceptives | X | X | | | | X | | | | | X | | | | X | | | | | | | | |
| requirements^{b,c} | | | | | | | | | | | | | | | | | | | | | | | |
| Inclusion/exclusion | X | X | | | | | | | | | | | | | | | | | | | | | |
| criteria | | | | | | | | | | | | | | | | | | | | | | | |
| Medical history | X | X | | | | | | | | | | | | | | | | | | | | | |
| (including HIV | | | | | | | | | | | | | | | | | | | | | | | |
| history and risk | | | | | | | | | | | | | | | | | | | | | | | |
| factors for clotting | | | | | | | | | | | | | | | | | | | | | | | |
| events and | | | | | | | | | | | | | | | | | | | | | | | |
| thrombocytopenia^{d}) | | | | | | | | | | | | | | | | | | | | | | | |
| Demographics (sex, | X | | | | | | | | | | | | | | | | | | | | | | |
| race/ethnicity, age) | | | | | | | | | | | | | | | | | | | | | | | |
| Height | X | | | | | | | | | | | | | | | | | | | | | | |
| Weight | X | X | | | X | X | | | X | X | X | | X | X | X | | X | X | X | X | X | X | X |
| Follicle-stimulating | X | | | | | | | | | | | | | | | | | | | | | | |
| hormone^{c} | | | | | | | | | | | | | | | | | | | | | | | |
| Serum pregnancy | X | | | | | | | | | | | | | | | | | | | | | | |
| testing^{c} | | | | | | | | | | | | | | | | | | | | | | | |
| Urine pregnancy | | X | | | | X | | | | X | X | | | X | X | | | X | X | X | X | X | X |
| testing^{c} | | | | | | | | | | | | | | | | | | | | | | | |
| Plasma HIV-1 RNA | X | X | | | X | X | | | X | X | X | | X | X | X | | X | X | X | X | X | X | X |
| Serology: HBV, | X | | | | | | | | | | | | | | | | | | | | | | |
| HCV^{e} | | | | | | | | | | | | | | | | | | | | | | | |
| Complete physical | X | | | | | | | | | | | | | | | | | | | | | X | X |
| examination | | | | | | | | | | | | | | | | | | | | | | | |
| Symptom-driven | | X | X | X | X | X | X | X | X | X | X | X | X | X | X | X | X | X | X | X | X | | |
| physical | | | | | | | | | | | | | | | | | | | | | | | |
| examination | | | | | | | | | | | | | | | | | | | | | | | |
| Standard 12-lead | X | | | | | | | | | | | | | | | | | | | | | | |
| ECG | | | | | | | | | | | | | | | | | | | | | | | |
| Vital signs^{f} | X | X | X | X | X | X | X | X | X | X | X | X | X | X | X | X | X | X | X | X | X | X | X |
| Hematology^{g} | X | X | X | | X | X | X | | X | X | X | | X | | X | X | X | | X | X | X | X | X |
| Coagulation panel, | X | X | X | | X | X | X | | X | X | X | X | X | | X | X | X | | X | X | X | X | X |
| including aPTT | | | | | | | | | | | | | | | | | | | | | | | |
| Clinical chemistry^{h} | X | X | | | X | | X | | X | X | X | X | X | | X | X | X | | X | | X | X | X |
| Urinalysis (dipstick)ⁱ | X | X | | | | | | | | X | X | | | X | X | | | X | X | X | X | X | X |
| CD4+/CD8+ T-cell | X | X | | | | | | | | | X | | | | X | | | | X | | X | X | X |
| counts^{j} | | | | | | | | | | | | | | | | | | | | | | | |
| Creatinine | X | X | X | | X | X | X | | X | X | X | X | X | | X | X | X | | X | X | X | X | X |
| clearance^{k} | | | | | | | | | | | | | | | | | | | | | | | |
| Adverse events¹ | X | X | X | X | X | X | X | X | X | X | X | X | X | X | X | X | X | X | X | X | X | X | X |
| Concomitant | X | X | X | X | X | X | X | X | X | X | X | X | X | X | X | X | X | X | X | X | X | X | X |
| medication review | | | | | | | | | | | | | | | | | | | | | | | |
| Dispense participant | | X | | | | X | | | | | X | | | | X | | | | | | | | |
| diary^{m} | | | | | | | | | | | | | | | | | | | | | | | |
| Review participant | | | | | X | | | | X | | | | X | | | | X | | | | | | |
| diary^{m} | | | | | | | | | | | | | | | | | | | | | | | |
| Randomization | | X | | | | | | | | | | | | | | | | | | | | | |
| ChAd or placebo | | X | | | | | | | | | | | | | X | | | | | | | | |
| administrationⁿ | | | | | | | | | | | | | | | | | | | | | | | |
| SAM or placebo | | X | | | | X | | | | | X | | | | | | | | | | | | |
| administration^{o} | | | | | | | | | | | | | | | | | | | | | | | |
| Blood samples for | | X | | | X | X | | | X | X | X | | X | X | X | | X | X | X | | X | | |
| immunogenicity | | | | | | | | | | | | | | | | | | | | | | | |
| assessment (IFNγ | | | | | | | | | | | | | | | | | | | | | | | |
| ELISpot assay) | | | | | | | | | | | | | | | | | | | | | | | |
| Whole blood | | X | | | | | | | | | | | | | | | X | | | | X | | |
| derived PBMC | | | | | | | | | | | | | | | | | | | | | | | |
| samples for latent | | | | | | | | | | | | | | | | | | | | | | | |
| HIV-1 reservoir | | | | | | | | | | | | | | | | | | | | | | | |
| assessment | | | | | | | | | | | | | | | | | | | | | | | |
| Whole blood | | X | | | | | | | | | | | | | | | X | | | | X | | |
| derived plasma and | | | | | | | | | | | | | | | | | | | | | | | |
| PBMC samples for | | | | | | | | | | | | | | | | | | | | | | | |
| active HIV-1 | | | | | | | | | | | | | | | | | | | | | | | |
| reservoir assessment | | | | | | | | | | | | | | | | | | | | | | | |
| Blood samples for | | X | | | | X | | | X | | | | X | | | | X | | | | X | | |
| HIV-1-specific T- | | | | | | | | | | | | | | | | | | | | | | | |
| cell phenotype and | | | | | | | | | | | | | | | | | | | | | | | |
| function | | | | | | | | | | | | | | | | | | | | | | | |
| Blood samples for | | X | X | X | X | X | X | X | X | | X | | X | | X | X | X | | | | | | |
| peripheral soluble | | | | | | | | | | | | | | | | | | | | | | | |
| proteins | | | | | | | | | | | | | | | | | | | | | | | |
| Blood samples for | | X | | | | X | | | | X | X | | | X | X | | X | | | | X | | |
| antivector immune | | | | | | | | | | | | | | | | | | | | | | | |
| responses^{p} | | | | | | | | | | | | | | | | | | | | | | | |
| Blood sample | As needed | | | | | | | | | | | | | | | | | | | | | | |
| collected at time of | | | | | | | | | | | | | | | | | | | | | | | |
| any clotting event^{q} | | | | | | | | | | | | | | | | | | | | | | | |
| HIV-1 | As needed | | | | | | | | | | | | | | | | | | | | | | |
| genotype/phenotype^{r} | | | | | | | | | | | | | | | | | | | | | | | |
| Blood sample for | | X | | | | | | | | | | | | | | | | | | | | | |
| HLA-genotype | | | | | | | | | | | | | | | | | | | | | | | |
| assessment^{s} | | | | | | | | | | | | | | | | | | | | | | | |

ALP = alkaline phosphatase; ALT = alanine aminotransferase; aPTT = activated partial thromboplastin time; AST = aspartate aminotransferase; BUN = blood urea nitrogen; CBC = complete blood count; CK = creatine kinase; CLcr = creatinine clearance; CPK = creatine phosphokinase; ECG = electrocardiogram; ELISA = enzyme-linked immunosorbent assay; ESDD = early study drug discontinuation; ET = early termination; FSH = follicle-stimulating hormone; HBV = hepatitis B virus; HCV = hepatitis C virus; HIV = human immunodeficiency virus; HLA = human leukocyte antigen; IFNγ ELISpot = interferon gamma enzyme-linked immunospot; INR = international normalized ratio; PBMC = peripheral blood mononuclear cell; PF4 = platelet factor 4; PT = prothrombin time; SCR = screening; TTS = thrombosis with thrombocytopenia syndrome
^{a}Participants that discontinue study treatment early are required to complete days.
^{b}Participants of childbearing potential must agree to follow study contraceptive requirements at screening. During the treatment period, contraceptive requirements are reviewed on dosing days.
^{c}Required for female participants of childbearing potential.
^{d}The master list of questions for evaluation of TTS.
^{e}Serology: HBV surface antigen and HCV antibody and reflex to HCV RNA.
^{f}Vital signs include pulse, blood pressure, and body temperature.
^{g}Hematology: complete blood count with differential and platelets.
^{h}Clinical chemistry (serum or plasma): ALP, AST, ALT, total bilirubin, direct and indirect bilirubin, total protein, albumin, bicarbonate, BUN, calcium, chloride, creatinine, glucose, phosphorus, magnesium, potassium, sodium, CPK/CK, and uric acid.
ⁱUrinalysis: color and clarity, specific gravity, pH, glucose, protein, blood and microscopic (reflex if positive), leukocyte esterase and microscopic (reflex if positive)
^{j}CD4+/CD8+ T-cell counts: CD4+ and CD8+ T-cell absolute count and percentage, and CD4+/CD8+ ratio.
^{k}Creatinine clearance to be calculated according to the Cockcroft-Gault formula for creatinine clearance: male: [(140 - age in years) × (weight in kg)]/[72 × (serum creatinine in mg/dL)] = CLcr (mL/min); female: [(140 − age in years) × (weight in kg) × 0.85]/[72 × (serum creatinine in mg/dL)] = CLcr (mL/min).
¹Participants are reminded to report all symptoms, including flu-like symptoms (e.g., fatigue, pyrexia, chills, myalgia, joint pain, headache, nausea, arthralgia), local events (e.g., injection-site pain, swelling, and erythema), or other unexpected symptoms. Adverse events of special interest, including TTS, should be reported).
^{m}Participants are asked to record potential vaccine reactions based on solicited events via diary for 7 days following each administration of study drug. Review of participant reactogenicity data occurs at the next visit that is greater than 7 days postdose.
ⁿParticipants in the active group receive a ChAd prime at Day 1 (Week 0) and a ChAd boost at Day 169 (Week 24).
^{o}Participants in the active group receive SAM boosts at Day 29 (Week 4) and Day 99 (Week 14).
^{p}Excess sample from baseline collection for antivector immune responses may be used for comparison of PF4 antibody levels with sample collected at the time of a clotting event.
^{q}Sample is collected at the time of any clotting event and compared to serum collected at baseline for antivector immune responses. Laboratory analyses to be performed should include but not limited to CBC with manual count as indicated, PF4 antibody ELISA, PT/INR, aPTT, fibrinogen, and D-dimer.
^{r}HIV-1 genotype and phenotype testing for confirmation of virologic resistance in case of virologic failure.
^{s}This sample should be collected on Day 1, before administration of the first dose of study drug, but may be collected at any time during the study, if necessary.

### Objectives and End Points

A primary objective of this study is to evaluate the safety and tolerability of HIV vaccine regimens comprising an adenovirus vector and a self-amplifying mRNA (SAM) (e.g., ChAd-01 and SAM-01 or ChAd-02 and SAM-02) in people with HIV-1 ([PWH]; Clade B) on antiretroviral therapy (ART) who are virologically suppressed.

A secondary objective of this study is to evaluate the immunogenicity of HIV vaccine regimens an adenovirus vector and a self-amplifying mRNA (SAM) (e.g., ChAd-01 and SAM-01 or ChAd-02 and SAM-02) in PWH (Clade B) on ART who are virologically suppressed.

A primary endpoint of this study is the incidence of adverse events and graded clinical laboratory abnormalities.

A secondary endpoint of this study is to determine the proportion of participants with vaccine-induced HIV-1-specific T-cell response, as determined by interferon gamma (IFNγ) enzyme-linked immunospot (ELISpot) assay and magnitude of the total vaccine-induced HIV-1-specific T-cell response, as determined by IFNγ ELISpot assay.

### Diagnosis and Main Eligibility Criteria:

People with HIV-1 (Clade B) who meet the following criteria are eligible for the study:
Age ≥18 and ≤60 years
Plasma HIV-1 RNA levels <50 copies/mL at the screening visit
Documented plasma HIV-1 RNA <50 copies/mL for ≥12 consecutive months prior to the screening visit:
   Using all available viral load determinations ≥12 consecutive months prior to screening of viral suppression on ART, HIV-1 RNA viral load must be below the limit of detection (<50 copies/mL) with ≤2 unconfirmed detectable (≤1000 copies/mL) HIV-1 RNA measurements
Participants who meet any of the following criteria are not eligible for the study:
Participants with coinfection and/or immunosuppression as described below:
   Autoimmune disease requiring ongoing immunosuppression (except in the case of mild autoimmune disease which requires topical treatments only)
   Evidence of chronic hepatitis B virus (HBV) infection (defined as positive hepatitis B surface antigen OR positive hepatitis B core antibody and negative hepatitis B surface antibody)
   Evidence of active hepatitis C virus (HCV) infection (defined as positive hepatitis C antibody and HCV RNA above lower limit of quantitation). Note: positive anti-HCV antibody and negative HCV polymerase chain reaction results are acceptable.
   Documented history of pre-ART CD4+ T-cell count nadir <100 cells/μL (unknown pre-ART CD4+ T-cell count nadir is acceptable)
   History of opportunistic illness indicative of Stage 3 HIV
   Acute febrile illness within 4 weeks prior to the first dose
Have received any vaccine (including messenger RNA or lipid nanoparticle based severe acute respiratory syndrome coronavirus 2 [SARS-CoV-2] vaccines) or immunomodulatory medication within 4 weeks prior to screening
Have previously received an HIV vaccine
Have received any ChAd-01 or ChAd-02 previously or other adenovirus-vectored vaccine (including adenovirus-vectored SARS-CoV-2 vaccines) within the last 6 months prior to screening
Have history of an antidrug antibody response to a therapeutic agent

### Exclusion Criteria

Participants who meet any of the following exclusion criteria are not eligible to be enrolled in this study:
1) Participants with coinfection and/or immunosuppression as described below:
   Autoimmune disease requiring ongoing immunosuppression (except in the case of mild autoimmune disease which requires topical treatments only)
   Evidence of chronic hepatitis B virus (HBV) infection (defined as positive hepatitis B surface antigen OR positive hepatitis B core antibody and negative hepatitis B surface antibody)
   Evidence of active hepatitis C virus (HCV) infection (defined as positive hepatitis C antibody and HCV RNA above lower limit of quantitation [LLOQ]). Note: positive anti-HCV antibody and negative HCV polymerase chain reaction results are acceptable.
   Documented history of pre-ART CD4+ T-cell count nadir <100 cells/μL (unknown pre-ART CD4+ T-cell count nadir is acceptable)
   History of opportunistic illness indicative of Stage 3 HIV
   Acute febrile illness within 4 weeks prior to the first dose
2) Have received any vaccine (including mRNA or LNP based SARS-CoV-2 vaccines) or immunomodulatory medication within 4 weeks prior to screening
3) Have previously received an HIV vaccine
4) Have received any GS-1966 previously or other adenovirus-vectored vaccine (including adenovirus-vectored SARS-CoV-2 vaccines) within the last 6 months prior to screening
5) Have history of an antidrug antibody response to a therapeutic agent
6) Have current alcohol or substance abuse judged by the investigator to potentially interfere with participant's compliance or participant's safety
7) Have poor venous access that limits phlebotomy
8) Have been treated with systemic steroids, immunosuppressant therapies, or chemotherapeutic agents within 3 months prior to screening or are expected to receive these agents during the study (e.g., corticosteroids, immunoglobulins, and other immune- or cytokine-based therapies)
9) Have previous or current receipt of humanized or human monoclonal antibodies, or polyclonal immunoglobulin
10) Have a history of any of the following:
   a) Significant serious skin disease, such as but not limited to rash, food allergy, eczema, psoriasis, or urticaria
   b) Significant drug sensitivity or drug allergy (such as anaphylaxis or hepatoxicity). Any history of anaphylaxis and related symptoms such as hives, respiratory difficulty, or angioedema
   c) Known hypersensitivity to the study drugs, metabolites, or formulation excipients
   d) Prior or current bleeding or platelet disorder, including, but not limited to, unexplained acute or chronic thrombocytopenia, family or personal history of bleeding or thrombosis, on medications associated with increased risk of bleeding or thrombosis, have conditions known to increase risk of thrombosis (e.g., post-partum, extreme obesity, active malignancy or post-transplantation, recent surgery, immobility, recent infection, recent head trauma, autoimmune disease, inflammatory disease), history of heparin-related thrombotic events, and/or receiving heparin treatments
   e) Uncontrolled/untreated autoimmune diseases including type 1 diabetes mellitus
11) Positive serum pregnancy test
12) Breastfeeding female
13) Requirement for ongoing therapy with, or prior use of, any prohibited medications
14) Have any serious or active medical or psychiatric illness (including depression) that, in the opinion of the investigator, would interfere with participant's treatment, assessment, or compliance with the protocol, including the following: renal, cardiac, hematological, hepatic, pulmonary (including chronic asthma), endocrine (including diabetes), central nervous, gastrointestinal (including an ulcer), vascular, metabolic (thyroid disorders, adrenal disease), and immunodeficiency disorders other than HIV-1 infection, active infection, or malignancy that are clinically significant or require treatment
15) Participation in any other clinical study (including observation studies) without prior approval from the sponsor is prohibited while participating in this study

### Prior and Concomitant Medications

Participants are required to continue taking their ART during this study.

Contraception is required.

Given the frequent observation of fever, participants are recommended to receive prophylactic antipyretic treatment with acetaminophen or ibuprofen prior to or following completion of study drug dosing to potentially limit the occurrence of this treatment-related AE.

Medications that are prohibited or are to be used with caution are listed in Table 10. Should participants have a need to initiate treatment with any prohibited concomitant medication, the study medical monitor must be consulted, and approval granted before initiation of the new medication. In instances where a prohibited medication is initiated before discussion with the study medical monitor, the investigator must notify the Study Sponsor as soon as he/she is aware of the use of the prohibited medication.

TABLE 10
Prior and Concomitant Medications That Are Prohibited or That Are to Be Used With Caution
Because of Impact on Participant Safety or Markers of Potential Efficacy Assessments

| | Medications to Be Used With | |
|---|---|---|
| Medication Class | Caution | Prohibited Medications |
| Vaccine | Elective vaccination (e.g., | Any vaccine within 4 weeks prior to |
| | influenza, SARS-CoV-2, hepatitis | screening |
| | A or B vaccine) during the course | Previously received HIV vaccine |
| | of the study requires prior approval | Any ChAd previously received or other |
| | from the sponsor | adenovirus-vectored vaccine within 6 |
| | | months prior to screening |
| Immunosuppression | Topical treatments for autoimmune | Systemic steroids, immunosuppressant |
| | disease during the course of the | therapies, or chemotherapeutic agents |
| | study | within 3 months prior to screening or |
| | | participant is expected to receive these |
| | | agents during the study (e.g., |
| | | corticosteroids, immunoglobulins, and |
| | | other immune- or cytokine-based |
| | | therapies) |
| Immunoglobulins | | Previous or current receipt of |
| | | humanized or human mAbs, or |
| | | polyclonal immunoglobulin |

HIV = human immunodeficiency virus;
mAbs = monoclonal antibodies;
SARS-CoV-2 = severe acute respiratory syndrome coronavirus 2

Study Procedures/Frequency: The study procedures to be conducted for each participant screened or enrolled in the study are presented in tabular form in Table 9.

Study Drug, Dose, and Mode of Administration:

Participants in each cohort receive either study drugs or placebo administered by intramuscular (IM) injection, according to randomized treatment assignment.

The study drugs are:
ChAd-01 or ChAd-02 up to 5×10¹¹ viral particles (vp) (Cohort 1:2.5×10¹¹ vp, monovalent; Cohort 2: 5×10¹¹ vp, monovalent; Cohort 3: up to 5×10¹¹ vp, bivalent)
SAM-01 or SAM-02 up to 60 μg (Cohort 1:30 μg, monovalent; Cohort 2:60 μg, monovalent; Cohort 3: up to 60 μg, bivalent)

Reference Therapy, Dose, and Mode of Administration: Placebo is sterile saline administered by IM injection in equal volume to corresponding study drug.

### ChAd and SAM

The HIV-1 immunogen covers 12 conserved regions across HIV-1 Clade B Group-specific antigen (Gag), Polymerase, and accessory protein Negative Regulatory Factor. The conserved regions are polypeptide regions (typically >15 amino acids) within these viral proteins and are concatenated to make a single immunogen. Two different immunogen sequences were designed (immunogens 1A and 1B) for use in a bivalent vaccine. Immunogens 1A and 1B are identical in structure (i.e., containing the same conserved regions), with different amino acids at certain positions introduced to cover more than 80% of potential human leukocyte antigen (HLA) class I epitopes among circulating Clade B viruses. Where applicable, immunogens 1A and 1B are expressed from 2 separate ChAd or SAM vectors such that ChAd-01 and SAM-01 encode HIV-1 immunogen 1A (corresponding to SEQ ID NO: 524) and ChAd-02 and SAM-02 encode HIV-1 immunogen 1B (corresponding to SEQ ID NO: 525).

ChAd is a replication-defective, E1, E3, and E4ORF2-4 deleted adenoviral vector based on ChAd68 (originally designated as Pan 9) that expresses HIV-1 immunogen sequences that were inserted into the deleted viral E1 region.

The SAM vector is based on Venezuelan equine encephalitis virus (VEEV), an alphavirus, with HIV-1 immunogen sequences replacing the structural proteins of VEEV. Without the structural proteins, no infectious viral particle is formed. The SAM is then formulated using lipid nanoparticles (LNP) to encapsulate and generate the SAM.

ChAD and SAM vectors are utilized as a heterologous prime/boost regimen to induce strong T-cell responses against HIV-1 antigens, resulting in the elimination of HIV-1-infected cells. The use of ChAd-01 or ChAd-02 overcomes the limitation of pre-existing immunity to human-derived adenoviral vectors, such as human adenovirus 5, which may diminish the immunogenicity of adenoviral-based vaccines (Roberts, et al., Hexon-chimaeric adenovirus serotype 5 vectors circumvent pre-existing anti-vector immunity. Nature 2006; 441 (7090): 239-43). Although using ChAd as a prime vaccination is anticipated to induce robust T-cell responses within 2 to 3 weeks, this also induces neutralizing antibodies against the ChAd structural coat proteins, as described for other adenovirus-based vectors (Ahi, et al., Adenoviral vector immunity: its implications and circumvention strategies. Curr Gene Ther 2011; 11 (4): 307-20), which is anticipated to blunt the potential of subsequent ChAd administrations to boost the vaccine-induced immune response if administered utilizing a short interval of 5 months or less from the first dose (Priddy, et al., Safety and Immunogenicity of a Replication-Incompetent Adenovirus Type 5 HIV-1 Clade B_gag/pol/nef Vaccine in Healthy Adults. Clin Infect Dis 2008; 46:1769-81). Thus, a heterologous prime/boost strategy is employed using ChAd-01 or ChAd-02, which is distinct from ChAd68 and can boost and sustain T-cell responses after multiple administrations. Preclinical study using simian immunodeficiency virus (SIV) antigens demonstrated that a ChAd/SAM prime/boost elicits strong T-cell responses in rhesus macaques. The safety and immunogenicity of this platform has also been demonstrated in the setting of oncology neoantigen vaccine clinical trials, which makes it an attractive candidate for testing HIV remission/cure combination strategies.

### Rationale for This Study

This is a Phase 1b, randomized, multicenter, single-blind, placebo-controlled study to evaluate the safety, tolerability, and immunogenicity of the ChAd+SAM prime/boost HIV-1 vaccine regimen in virologically suppressed PWH who are on ART to enable the further development of this product as part of a combination treatment strategy for HIV-1 long-term remission/cure. Participants maintain ART treatment for the duration of the study.

Preclinical studies of the ChAd/SAM platform with SIV immunogens have demonstrated the induction of robust, broad, and durable SIV-specific CD8+ T-cell responses in the nonhuman primate (NHP) model. These T-cell responses were primarily from CD8+ T cells and the induced CD8+ T cells were polyfunctional (ie, secrete multiple cytokines). Since functional CD8+ T-cell responses correlate with control of HIV and SIV (Jones and Walker, HIV-specific CD8 (+) T cells and HIV eradication. J Clin Invest 2016; 126 (2): 455-63), ChAd and SAM with HIV immunogens may contribute to establishing immune control of HIV-1 infection and would, therefore, represent a key component of an HIV-1 remission/cure regimen.

### Rationale for Dose Selection of ChAd and SAM

This first in human study evaluates 2 escalating dose levels of ChAd and SAM as a monovalent vaccine (ChAd-02+SAM-02), then 1 dose level of ChAd and SAM as a bivalent vaccine (ChAd-01 and ChAd-02+SAM-01 and SAM-02). The 2 ChAd dose levels are 2.5×10¹¹ viral particles (vp) and up to 5×10¹¹ vp and the 2 SAM dose levels are 30 μg and up to 60 μg. Similar or higher ChAd and SAM doses of cancer vaccines (ChAd/SAM: GRT-C901/GRT-R902 and GRT-C903/GRT-R904) are being evaluated in early-phase oncology studies. Furthermore, ChAd doses of 1×10¹² vp and SAM doses of 300 μg were well tolerated in naive Indian rhesus macaques, where both the ChAd and SAM vectors encoded the full-length Gag antigen of SIV strain smE543.

### Formulation

ChAd-01 and ChAd-02 are sterile, HIV-1 therapeutic vaccine primes for IM administration. Each is formulated as a suspension in an aqueous buffer containing 5 mM Tris, 5% sucrose, 75 mM sodium chloride, and 1 mM magnesium chloride, at pH 8.0. The product is formulated to achieve a nominal concentration of 5×10¹¹ vp/mL and is provided in a volume of 1.2 mL per vial.

SAM-01 and SAM-02 are sterile, HIV-1 therapeutic vaccine boosts for IM administration. Each is formulated as SAM in a suspension of LNPs in an aqueous buffer containing 5 mM Tris, 10% sucrose, and 10% maltose, at pH 8.0. The product is formulated to deliver 0.2 mg of SAM per mL of suspension and is provided in a volume of 0.7 mL per vial.

### Storage and Handling

ChAd should be thawed at ambient temperature (20° C. to 25° C.) and mixed by gently inverting the vial and should not be filtered prior to administration. Once thawed, the product should appear as a clear, colorless to slightly blue-grey solution, essentially free from particles. Do not vortex.

Adenovirus vectors such as ChAd-01 and ChAd-02 are classified as biosafety level (BSL)-2 agents and therefore should only be manipulated in BSL-2 facilities by trained staff wearing appropriate personal protective equipment.

SAM should be thawed at ambient temperature (20° C. to 25° C.) and mixed by gently inverting the vial. Once thawed, the product should appear as a white to off-white, homogenous, opalescent liquid. Do not vortex.

During dose-finding studies, SAM-01 and SAM-02 may be diluted using 0.9% saline for injection (preservative-free). Dose strength refers to the dose of SAM delivered in the LNPs (i.e., a dose of 30 μg is 30 μg of SAM formulated in LNPs).

Since the SAM vector does not form a virus particle (no structural genes are encoded), it can be handled under BSL-1 conditions by trained staff wearing appropriate personal protective equipment.

### Example 5

### Heterologous ChAd/samRNA SIV Vaccine Induces Robust T Cell Responses in Macaques

Background: Since the latent HIV reservoir persists on antiretroviral therapy (ART), additional interventions are needed to achieve remission. Therapeutic vaccination to enhance HIV-specific T cell immunity is a focal strategy in the pursuit of an HIV cure. Here, we evaluated safety and immunogenicity of a heterologous SIV vaccine in combination with immune modulators in macaques.

Methods: Naive rhesus macaques (n=6/group) were immunized with a chimpanzee adenovirus (ChAd) and self-amplifying mRNA (samRNA)-based vaccine alone or in combination with either an anti-PD-1 antibody (aPD-1, which is the simianized version of nivolumab), anti-CTLA4 antibody (aCTLA4, which is ipilimumab) or FLT3R agonist (FLT3Ra, which is a simianized version of GS-3583). ChAd and samRNA vectors encoding full length SIV Gag, Pol and Env antigens were administered intramuscularly on weeks 0, 24 (ChAd) and 4, 12, 42 (samRNA). aPD-1 and aCTLA4 were dosed subcutaneously and/or intravenously concomitant with immunization. FLT3Ra was dosed intravenously 1 week prior to immunization. Immunogenicity was characterized by IFNγ ELISpot and multi-parameter flow cytometry.

Materials: The ChAd-based vaccine comprised three ChAd vector constructs, the first comprising a nucleotide sequence encoding the SIV Gag insert, the second comprising a nucleotide sequence encoding the SIV Env insert, and the third comprising a nucleotide sequence encoding the SIV Pol insert. The chimpanzee adenovirus (ChAd) construct comprising the SIV Gag insert is shown in SEQ ID NO: 534 (ChAd68-CMV-SIVGag-SV40 PolyA). The chimpanzee adenovirus (ChAd) construct comprising the SIV Env insert is shown in SEQ ID NO: 535 (ChAd68-CMV-SIVEnv-SV40 PolyA). The ChAd construct encoding SIV Pol was constructed by replacing nucleotides 1256 to 3358 (e.g., SIV Env insert) of SEQ ID NO: 535 with SEQ ID NO: 536 (e.g., SIV Pol insert). The samRNA-based vaccine comprised three samRNA vectors, the first comprising a nucleotide sequence encoding the SIV Gag insert, the second comprising a nucleotide sequence encoding the SIV Env insert, and the third comprising a nucleotide sequence encoding the SIV Pol insert. The self-amplifying mRNA (samRNA) construct comprising the SIV Env insert is shown in SEQ ID NO: 537. The samRNA construct comprising the SIV Pol insert was constructed by replacing nucleotides 7571 to 9673 (e.g., SIV Env insert) of SEQ ID NO: 537 with SEQ ID NO: 538 (e.g., SIV Gag insert). The samRNA construct comprising the SIV Pol insert was constructed by replacing nucleotides 7571 to 9673 (e.g., SIV Env insert) of SEQ ID NO: 537 with SEQI D NO: 536 (e.g., SIV Pol insert). The anti-PD-1 antibody (aPD-1) comprises the heavy chain sequence of SEQ ID NO: 531 and the light chain sequence of SEQ ID NO: 532. The anti-CTLA4 antibody comprises the amino acid sequence of ipilimumab. The FLT3R agonist is a homodimer, wherein each monomer comprises the amino acid sequence of SEQ ID NO: 533.

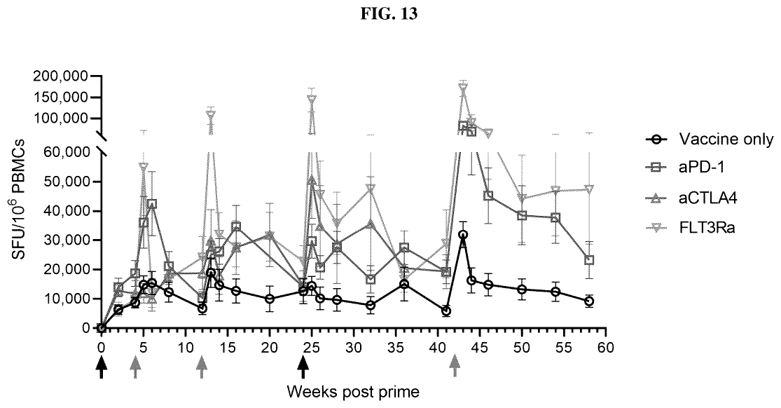

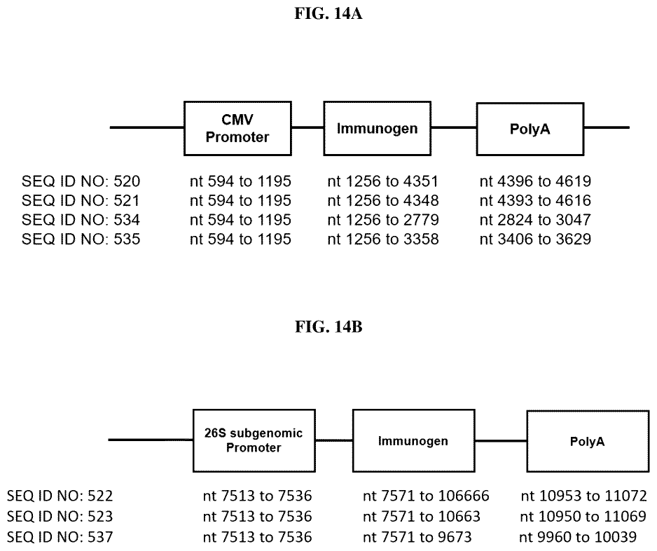

Results: All regimens were well tolerated with transient small increases in inflammatory markers and body temperature. As shown in FIG. 13, the vaccine elicited strong IFNγ ELISpot responses 4 weeks post-ChAd prime (8,724±1,845 SFU/106 PBMCs, mean±SEM), which were further augmented by boosts with samRNA (mean 1.8, 3.7, and 11.5-fold increase post each boost, respectively) and ChAd (1.7-fold increase). The responses were durable through at least 16 weeks post last immunization (range 4,277-27,514 SFU/10⁶ PBMCs). Combination with either aPD-1, aCTLA4 or FLT3Ra augmented T cell response magnitude (from peak of 32,234±4,433 to 89,228±9,850, 51,767±12,738, and 184,197±22,054 SFU/10⁶ PBMCs, mean±SEM, respectively). Responses were measured with overlapping peptides against all 3 SIV antigens and the breadth was greater in combination with aCTLA4 or FLT3Ra than with vaccine alone (p<0.05, week 13). Vaccine alone elicited predominantly CD8⁺ T-cell responses (1.9% IFNγ⁺/CD8⁺ and 0.6% IFNγ⁺/CD4⁺, week 13), while FLT3Ra also robustly enhanced CD4⁺ T cell responses (2.0% IFNγ+/CD8+ and 3.5% IFNγ+/CD4+, week 13). T cell responses were polyfunctional (predominantly IFNγ⁺ TNFα⁺) in all groups.

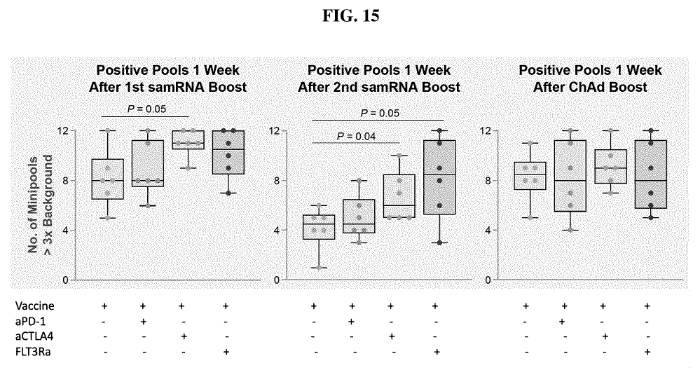

FIG. 15 shows the results of gag-specific T-responses in PBMCs as assessed by overnight IFNγ ELISpot using 12 overlapping peptide pools spanning Gag (~10 peptides/pool) at study weeks 5, 13, and 25. The number of minipools with response >3× background are shown for each animal. The data is presented as median (interquartile range [IQR]); Mann-Whitney test: only P values≤0.05 are shown. As shown in FIG. 15, the combination with aCTLA4 or FLT3Ra increased the breadth of T cell response.

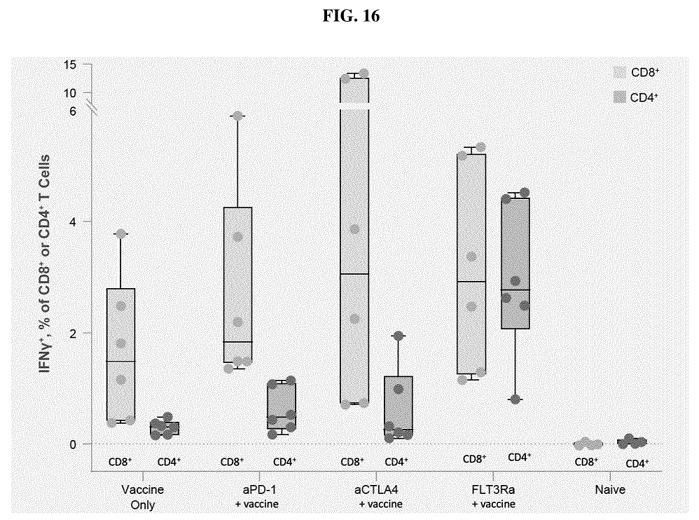

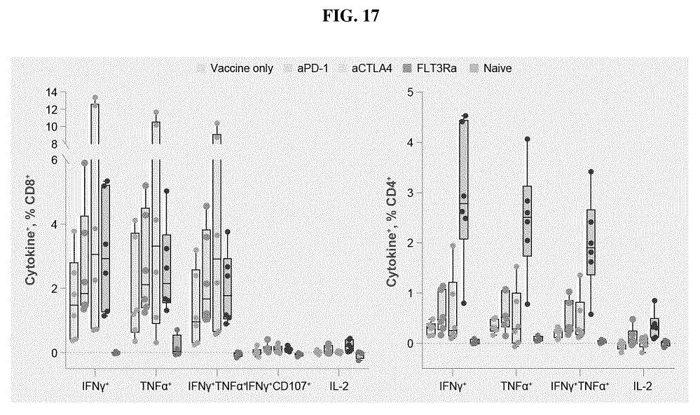

FIG. 16 and FIG. 17 show the results of intracellular cytokine staining of PBMCs at week 14 following overnight stimulation with 4 overlapping peptide pools spanning Gag, Pol, and Env, with background subtracted. The sum of pools are presented as median (IQR). As shown in FIG. 16, the vaccine drove predominantly CD8⁺ T-cell response and the combination with FLT3LRa also expanded CD4⁺ T-cell population. As shown in FIG. 17, the vaccine induced predominantly IFNγ+ TNFα+ polyfunctional T cells, which were increased by combination with aPD-1, aCTLA-4, or FLT3Ra.

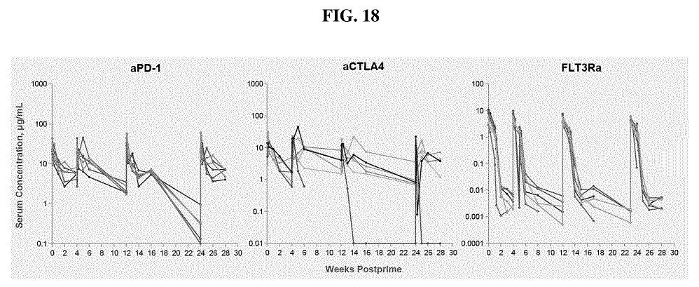

FIG. 18 shows the serum exposures levels in animals dosed with aPD-1, aCTLA4, and FLTRa as shown in Table 17. As shown in FIG. 18 serum exposures of aPD-1, aCTLA4, and FLT3Ra were consistent across animals and repeat doses. AR=accumulation ratio; AUC0-28d=area under serum concentration vs time curve from predose to 28 d postdose; Cmax=observed peak serum concentration.

TABLE 17

| | | Dose 1 | | Dose 2 | | |
|---|---|---|---|---|---|---|
| | Dose, | | AUC_{0-28 d}, | | AUC_{0-28 d}, | |
| Treatment | mg/kg | Cₘₐₓ, ug/mL | d * μg/mL | Cₘₐₓ, ug/mL | d * μg/mL | AR |
| aPD-1 | 3.0 | 31.8 ± 9.43 | 246 ± 58.2 | 33.5 ± 14.4 | 301 ± 81.2 | 1.2 |
| aCTLA4 | 9.1 | 13.9 ± 8.93 | 128 ± 36.6 | 11.9 ± 6.57 | 124 ± 64.9 | 1 |
| FLT3Ra | 0.3 | 6.53 ± 3.19 | 26.3 ± 13.6 | 5.23 ± 1.10 | 22.2 ± 6.53 | 0.8 |

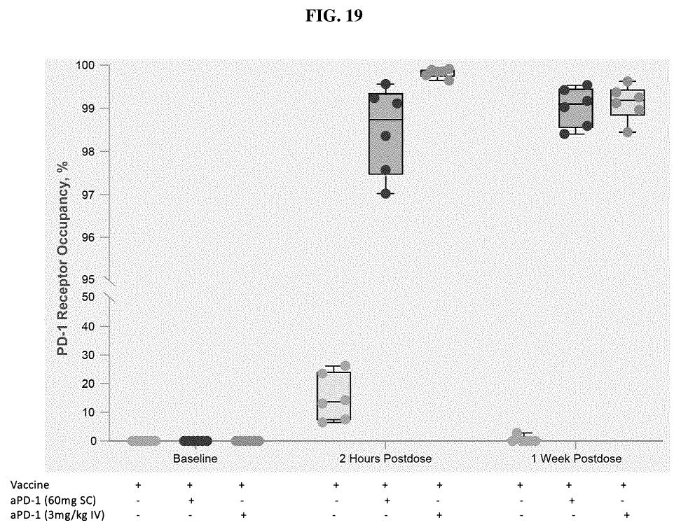

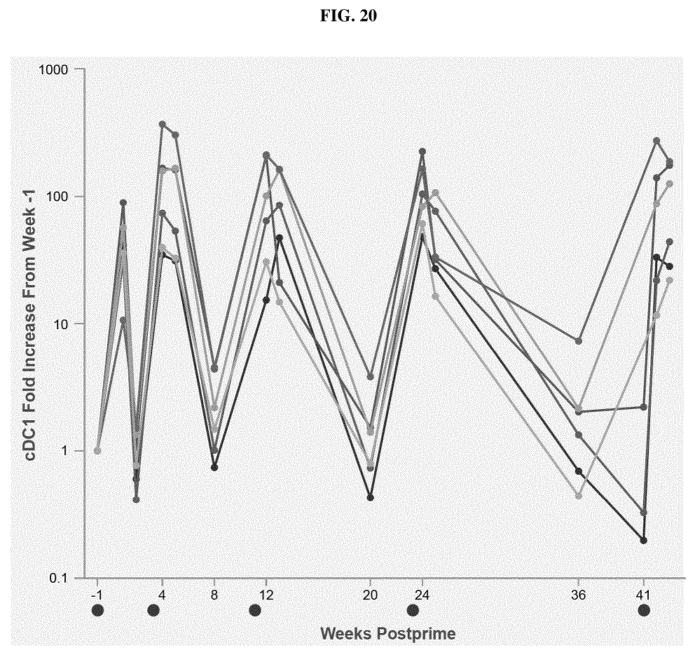

FIG. 19 shows the results of PD-1 receptor occupancy assay. PD-1 receptor occupancy was assessed by flow cytometry of PBMCs at specified time points following treatment with vaccine alone or in combination with aPD-1, wherein the aPD-1 was administered subcutaneously (SC, 60 mg) or intravenously (IV, 3 mg/kg). Results shown were normalized to baseline values and the data presented as median (IQR). As shown in FIG. 19, complete blockade of PD-1 receptor was observed following aPD-1 treatment via subcutaneous or intravenous route.

FIG. 20 shows cDC1 expansion in PBMCs from animals treated with vaccine in combination with FLT3Ra. Conventional type 1 dendritic cell (cDC1) population (defined as CD14−CD123−/CD11c+HLA-DR+/Clec9a+CD1c− of viable singlets) assessed in PBMCs for each animal by flow cytometry at specified time points; normalized to baseline value for each animal; purple circles indicate FLT3Ra dosing. As shown in FIG. 20, FLT3Ra in combination with vaccine led to expansion of cDC1 cells in PBMCs.

Conclusions: ChAd/samRNA heterologous SIV vaccine was well tolerated and stimulated robust and broad antigen-specific T cell responses. The immune responses were augmented by anti-PD-1 antibody, anti-CTLA4 antibody or FLT3R agonist warranting their further exploration as part of a combination therapeutic approach for HIV cure. T-cell responses were predominantly CD8+ and polyfunctional. Magnitude and/or breadth of SIV-specific T-cell responses were augmented by aPD-1, aCTLA4, or FLT3Ra.

SEQUENCE LISTING
(SEQ ID NO: 1)
Cys Ser Ala Thr Glu Lys Leu Trp Val Thr Val Tyr Tyr Gly Val Pro Val Trp Lys Glu Ala
Thr Thr Thr Leu
(SEQ ID NO: 2)
Leu Trp Val Thr Val Tyr Tyr Gly Val Pro Val Trp Lys Glu Ala
(SEQ ID NO: 3)
Leu Trp Val Thr Ile Tyr Tyr Gly Val Pro Val Trp Lys Asp Ala
(SEQ ID NO: 4)
Leu Trp Val Thr Val Tyr Tyr Gly Val Pro Val Trp Lys Glu
(SEQ ID NO: 5)
Leu Trp Val Thr Ile Tyr Tyr Gly Val Pro Val Trp Lys Asp
(SEQ ID NO: 6)
Val Thr Val Tyr Tyr Gly Val Pro Val
(SEQ ID NO: 7)
Val Thr Ile Tyr Tyr Gly Val Pro Val
(SEQ ID NO: 8)
Ala Thr Thr Thr Leu Phe Cys Ala Ser Asp Ala Lys Ala Tyr
(SEQ ID NO: 9)
Ala Asn Thr Thr Leu Phe Cys Ala Ser Asp Ala Lys Gly Tyr
(SEQ ID NO: 10)
Lys Ala Tyr Asp Thr Glu Val His Asn Val Trp Ala Thr His Ala Cys Val Pro Thr Asp
Pro Asn Pro Gln Glu
(SEQ ID NO: 11)
Ala His Asn Val Trp Ala Thr His Ala Cys Val Pro Thr Asp Pro Asn Pro Gln Glu
(SEQ ID NO: 12)
Val His Asn Ile Trp Ala Thr His Ala Cys Val Pro Thr Asp Pro Ser Pro Gln Glu
(SEQ ID NO: 13)
His Asn Val Trp Ala Thr His Ala Cys Val Pro Thr Asp Pro Asn Pro Gln Glu
(SEQ ID NO: 14)
His Asn Ile Trp Ala Thr His Ala Cys Val Pro Thr Asp Pro Ser Pro Gln Glu
(SEQ ID NO: 15)
Asn Val Trp Ala Thr His Ala Cys Val
(SEQ ID NO: 16)
Asn Ile Trp Ala Thr His Ala Cys Val
(SEQ ID NO: 17)
Asp Ile Ile Ser Leu Trp Asp Gln Ser Leu Lys Pro Cys Val Lys Leu Thr Pro Leu Cys Val
Thr Leu
(SEQ ID NO: 18)
Asp Ile Ile Ser Leu Trp Asp Glu Ser Leu Lys Pro Cys Val Lys Leu Thr Pro Ile Cys Val
Thr Leu
(SEQ ID NO: 19)
Asp Gln Ser Leu Lys Pro Cys Val Lys Leu Thr Pro Leu Cys Val Thr Leu Asn Cys Thr
Asp Leu Arg Asn Thr
(SEQ ID NO: 20)
Asp Glu Ser Leu Lys Pro Cys Val Lys Leu Thr Pro Ile Cys Val Thr Leu Asn Cys Thr
Asp Leu Arg Asn Thr
(SEQ ID NO: 21)
Lys Leu Thr Pro Leu Cys Val Thr Leu
(SEQ ID NO: 22)
Lys Leu Thr Pro Ile Cys Val Thr Leu
(SEQ ID NO: 23)
Ser Phe Glu Pro Ile Pro Ile His Tyr Cys Ala Pro Ala Gly Phe Ala Ile Leu
(SEQ ID NO: 24)
Thr Phe Glu Pro Ile Pro Ile His Tyr Cys Thr Pro Ala Gly Phe Ala Ile Leu
(SEQ ID NO: 25)
Pro Ala Gly Phe Ala Ile Leu Lys Cys
(SEQ ID NO: 26)
Pro Ala Gly Phe Ala Leu Leu Lys Cys
(SEQ ID NO: 27)
Gly Thr Gly Pro Cys Thr Asn Val Ser Thr Val Gln Cys Thr His Gly Ile Arg Pro Val Val
Ser Thr Gln Leu
(SEQ ID NO: 28)
Asn Val Ser Thr Val Gln Cys Thr His Gly Ile Arg Pro Val Val Ser Thr Gln Leu Leu Leu
Asn Gly Ser Leu Ala Glu Glu
(SEQ ID NO: 29)
Asn Ile Ser Thr Val Gln Cys Thr His Gly Ile Lys Pro Val Val Ser Thr Gln Leu Leu Leu
Asn Gly Ser Leu Ala Glu Lys
(SEQ ID NO: 30)
Ser Thr Val Gln Cys Thr His Gly Ile
(SEQ ID NO: 31)
Phe Asn Cys Gly Gly Glu Phe Phe Tyr Cys Asn
(SEQ ID NO: 32)
Phe Asn Cys Arg Gly Glu Phe Phe Tyr Cys Asn
(SEQ ID NO: 33)
Val Gly Lys Ala Met Tyr Ala Pro Pro Ile
(SEQ ID NO: 34)
Val Gly Arg Ala Met Tyr Ala Pro Pro Ile
(SEQ ID NO: 35)
Gly Gly Asp Met Arg Asp Asn Trp Arg Ser
(SEQ ID NO: 36)
Gly Gly Asn Met Lys Asp Asn Trp Arg Ser
(SEQ ID NO: 37)
Met Arg Asp Asn Trp Arg Ser Glu Leu Tyr Lys Tyr Lys Val Val
(SEQ ID NO: 38)
Met Lys Asp Asn Trp Arg Ser Glu Leu Tyr Arg Tyr Lys Val Val
(SEQ ID NO: 39)
Ala Lys Arg Arg Val Val Gln Arg Glu Lys Arg
(SEQ ID NO: 40)
Ala Arg Arg Arg Val Val Gln Arg Glu Lys Arg
(SEQ ID NO: 41)
Lys Arg Arg Val Val Gln Arg Glu Lys Arg Ala Val Gly Ile Gly Ala Met Phe Leu Gly
Phe Leu Gly Ala Ala Gly Ser Thr Met Gly Ala Ala Ser Ile Thr Leu Thr Val Gln Ala Arg Gln Leu
Leu Ser Gly Ile Val Gln Gln Gln Asn Asn Leu Leu Arg Ala Ile Glu Ala Gln Gln His Leu Leu Gln
Leu Thr Val Trp Gly Ile Lys Gln Leu Gln Ala Arg Val Leu Ala Val Glu Arg Tyr Leu Lys Asp
Gln Gln Leu Leu Gly Ile Trp Gly Cys Ser Gly Lys Leu Ile Cys Thr Thr
(SEQ ID NO: 42)
Arg Arg Arg Val Val Gln Arg Glu Lys Arg Ala Ile Gly Leu Gly Ala Val Phe Leu Gly
Phe Leu Gly Thr Ala Gly Ser Thr Met Gly Ala Ala Ser Met Thr Leu Thr Val Gln Ala Arg Leu
Leu Leu Ser Gly Ile Val Gln Gln Gln Ser Asn Leu Leu Arg Ala Ile Glu Ala Gln Gln His Met Leu
Gln Leu Thr Val Trp Gly Ile Lys Gln Leu Gln Ala Arg Ile Leu Ala Val Glu Arg Tyr Leu Arg Asp
Gln Gln Leu Leu Gly Ile Trp Gly Cys Ser Gly Arg Leu Ile Cys Thr Thr
(SEQ ID NO: 43)
Phe Leu Gly Phe Leu Gly Ala Ala Gly Ser Thr Met Gly Ala Ala Ser
(SEQ ID NO: 44)
Phe Leu Gly Phe Leu Gly Thr Ala Gly Ser Thr Met Gly Ala Ala Ala
(SEQ ID NO: 45)
Ala Ser Ile Thr Leu Thr Val Gln Ala Arg Gln Leu Leu Ser Gly Ile Val Gln Gln Gln Asn
Asn Leu Leu Arg Ala Ile Glu Ala Gln Gln His Leu Leu Gln Leu Thr Val Trp Gly Ile Lys Gln Leu
Gln Ala Arg Val Leu Ala Val Glu Arg Tyr Leu Lys Asp Gln Gln Leu Leu Gly Ile Trp Gly Cys
Ser Gly Lys Leu Ile Cys Thr Thr
(SEQ ID NO: 46)
Ala Ser Met Thr Leu Thr Val Gln Ala Arg Leu Leu Leu Ser Gly Ile Val Gln Gln Gln Ser
Asn Leu Leu Arg Ala Ile Glu Ala Gln Gln His Met Leu Gln Leu Thr Val Trp Gly Ile Lys Gln Leu
Gln Ala Arg Ile Leu Ala Val Glu Arg Tyr Leu Arg Asp Gln Gln Leu Leu Gly Ile Trp Gly Cys Ser
Gly Arg Leu Ile Cys Thr Thr
(SEQ ID NO: 47)
Thr Leu Thr Val Gln Ala Arg Gln Leu Leu Ser Gly Ile Val Gln Gln Gln Asn Asn Leu
Leu
(SEQ ID NO: 48)
Thr Leu Thr Val Gln Ala Arg Leu Leu Leu Ser Gly Ile Val Gln Gln Gln Ser Asn Leu
Leu
(SEQ ID NO: 49)
Asn Leu Leu Arg Ala Ile Glu Ala Gln Gln His
(SEQ ID NO: 50)
Asn Leu Leu Lys Ala Ile Glu Ala Gln Gln His
(SEQ ID NO: 51)
Ala Ile Glu Ala Gln Gln His Leu Leu Gln Leu Thr Val Trp Gly Ile Lys Gln Leu Gln Ala
Arg Val Leu Ala Val Glu
(SEQ ID NO: 52)
Ala Ile Glu Ala Gln Gln His Met Leu Gln Leu Thr Val Trp Gly Ile Lys Gln Leu Gln Ala
Arg Ile Leu Ala Val Glu
(SEQ ID NO: 53)
Glu Arg Tyr Leu Lys Asp Gln Gln Leu
(SEQ ID NO: 54)
Glu Arg Tyr Leu Arg Asp Gln Gln Leu
(SEQ ID NO: 55)
Tyr Leu Lys Asp Gln Gln Leu Leu Gly
(SEQ ID NO: 56)
Tyr Leu Arg Asp Gln Gln Leu Leu Gly
(SEQ ID NO: 57)
Tyr Leu Lys Asp Gln Gln Leu Leu Gly Ile Trp Gly Cys Ser Gly Lys Leu Ile Cys Thr Thr
Ala Val Pro Trp
(SEQ ID NO: 58)
Asp Gln Gln Leu Leu Gly Ile Trp Gly Cys Ser Gly Lys Leu Ile Cys Thr Thr
(SEQ ID NO: 59)
Asp Gln Gln Leu Leu Gly Leu Trp Gly Cys Ser Gly Lys Leu Ile Cys Pro Thr
(SEQ ID NO: 60)
Gly Ile Trp Gly Cys Ser Gly Lys Leu
(SEQ ID NO: 61)
Gly Leu Trp Gly Cys Ser Gly Lys Leu
(SEQ ID NO: 62)
Trp Leu Trp Tyr Ile Lys Ile Phe Ile Met Ile
(SEQ ID NO: 63)
Trp Leu Trp Tyr Ile Arg Ile Phe Ile Met Ile
(SEQ ID NO: 64)
Ile Phe Ile Met Ile Val Gly Gly Leu Ile Gly Leu Arg Ile
(SEQ ID NO: 65)
Leu Phe Ile Met Ile Val Gly Gly Leu Val Gly Leu Arg Ile
(SEQ ID NO: 66)
Val Asn Arg Val Arg Gln Gly Tyr Ser Pro Leu Ser Phe Gln Thr
(SEQ ID NO: 67)
Val Asn Arg Val Arg Lys Gly Tyr Ser Pro Leu Ser Phe Gln Ile
(SEQ ID NO: 68)
Met Gly Ala Arg Ala Ser Val Leu Ser Gly Gly
(SEQ ID NO: 69)
Met Gly Ala Arg Ala Ser Ile Leu Ser Gly Gly
(SEQ ID NO: 70)
Met Gly Ala Arg Ala Ser Val Leu Ser Gly Gly Glu Leu Asp Arg Trp
Glu Lys Ile Arg Leu Arg Pro Gly Gly Lys Lys Lys Tyr Arg Leu Lys His Ile Val Trp Ala Ser Arg
Glu Leu Glu Arg Phe Ala Val Asn Pro Gly Leu Leu Glu Thr
(SEQ ID NO: 71)
Met Gly Ala Arg Ala Ser Ile Leu Ser Gly Gly Lys Leu Asp Lys Trp
Glu Lys Ile Arg Leu Arg Pro Gly Gly Arg Lys Lys Tyr Lys Leu Lys His Ile Val Trp Ala Ser Arg
Glu Leu Glu Arg Phe Ala Val Asn Pro Gly Leu Leu Glu Thr
(SEQ ID NO: 72)
Leu Asp Arg Trp Glu Lys Ile Arg Leu Arg Pro Gly Gly
(SEQ ID NO: 73)
Leu Asp Lys Trp Glu Lys Ile Arg Leu Arg Pro Met Gly
(SEQ ID NO: 74)
Ile Arg Leu Arg Pro Gly Gly Lys Lys
(SEQ ID NO: 75)
Ile Arg Leu Arg Pro Gly Gly Arg Lys
(SEQ ID NO: 76)
Leu Lys His Ile Val Trp Ala Ser Arg Glu Leu Glu Arg Phe Ala Val
Asn Pro Gly Leu Leu Glu Thr
(SEQ ID NO: 77)
Leu Lys His Leu Val Trp Ala Ser Arg Glu Leu Glu Arg Phe Ala Leu
Asn Pro Gly Leu Leu Glu Thr
(SEQ ID NO: 78)
Ala Ser Arg Glu Leu Glu Arg Phe Ala Val Asn Pro Gly Leu Leu
(SEQ ID NO: 79)
Ala Ser Arg Glu Leu Glu Arg Phe Ala Leu Asn Pro Gly Leu Leu
(SEQ ID NO: 80)
Thr Gly Ser Glu Glu Leu Lys Ser Leu
(SEQ ID NO: 81)
Thr Gly Ser Glu Glu Leu Arg Ser Leu
(SEQ ID NO: 82)
Asp Thr Lys Glu Ala Leu Asp Lys Ile
(SEQ ID NO: 83)
Asp Thr Lys Glu Ala Leu Glu Lys Ile
(SEQ ID NO: 84)
Glu Ala Leu Asp Lys Ile Glu Glu Glu
(SEQ ID NO: 85)
Glu Ala Leu Glu Lys Ile Glu Glu Glu
(SEQ ID NO: 86)
Val Ser Gln Asn Tyr Pro Ile Val Gln Asn
(SEQ ID NO: 87)
Val Ser Gln Asn Phe Pro Ile Val Gln Asn
(SEQ ID NO: 88)
Pro Ile Val Gln Asn Leu Gln Gly Gln Met Val His Gln Ala Ile Ser Pro
Arg Thr Leu Asn Ala Trp Val Lys Val Val Glu Glu Lys Ala Phe Ser Pro Glu Val Ile Pro Met Phe
Ser Ala Leu Ser Glu Gly Ala Thr Pro Gln Asp Leu Asn Thr Met Leu Asn Thr Val Gly Gly His
Gln Ala Ala Met Gln Met Leu Lys Glu Thr Ile Asn Glu Glu Ala Ala Glu Trp Asp Arg Leu His
Pro Val His Ala Gly Pro Ile Ala Pro Gly Gln Met Arg Glu Pro Arg Gly Ser Asp Ile Ala Gly Thr
Thr Ser Thr Leu Gln Glu Gln Ile Gly Trp Met Thr Asn Asn Pro Pro Ile Pro Val Gly Glu Ile Tyr
Lys Arg Trp Ile Ile Leu Gly Leu Asn Lys Ile Val Arg Met Tyr Ser Pro Thr Ser Ile Leu Asp Ile
Arg Gln Gly Pro Lys Glu Pro Phe Arg Asp Tyr Val Asp Arg Phe Tyr Lys Thr Leu Arg Ala Glu
Gln Ala Ser Gln Glu Val Lys Asn Trp Met Thr Glu Thr Leu Leu Val Gln Asn Ala Asn Pro Asp
Cys Lys Thr Ile Leu Lys Ala Leu Gly Pro Ala Ala Thr Leu Glu Glu Met Met Thr Ala Cys Gln
Gly Val Gly Gly Pro Gly His Lys Ala Arg Val Leu
(SEQ ID NO: 89)
Pro Ile Val Gln Asn Ile Gln Gly Gln Met Val His Gln Pro Ile Ser Pro
Arg Thr Leu Asn Ala Trp Val Lys Val Ile Glu Glu Lys Ala Phe Ser Pro Glu Val Ile Pro Met Phe
Thr Ala Leu Ser Glu Gly Ala Thr Pro His Asp Leu Asn Thr Met Leu Asn Thr Ile Gly Gly His Gln
Ala Ala Met Gln Met Leu Lys Asp Thr Ile Asn Glu Glu Ala Ala Glu Trp Asp Arg Val His Pro
Val His Ala Gly Pro Val Ala Pro Gly Gln Met Arg Asp Pro Arg Gly Ser Asp Ile Ala Gly Thr Thr
Ser Asn Leu Gln Glu Gln Ile Gly Trp Met Thr Ser Asn Pro Pro Ile Pro Val Gly Asp Ile Tyr Lys
Arg Trp Ile Ile Met Gly Leu Asn Lys Ile Val Arg Met Tyr Ser Pro Val Ser Ile Leu Asp Ile Lys
Gln Gly Pro Lys Glu Pro Phe Arg Asp Tyr Val Asp Arg Phe Tyr Arg Thr Leu Arg Ala Glu Gln
Ala Ser Gln Asp Val Lys Asn Trp Met Thr Glu Thr Leu Leu Val Gln Asn Ser Asn Pro Asp Cys
Lys Thr Ile Leu Lys Ala Leu Gly Pro Gly Ala Thr Leu Glu Glu Met Met Ser Ala Cys Gln Gly Val
Gly Gly Pro Ser His Lys Ala Arg Val Leu
(SEQ ID NO: 90)
Met Val His Gln Ala Ile Ser Pro Arg Thr Leu Asn Ala Trp Val Lys
Val Val Glu Glu Lys Ala Phe Ser Pro
(SEQ ID NO: 91)
Met Val His Gln Pro Ile Ser Pro Arg Thr Leu Asn Ala Trp Val Lys
Val Ile Glu Glu Lys Ala Phe Ser Pro
(SEQ ID NO: 92)
Ile Ser Pro Arg Thr Leu Asn Ala Trp Val Lys Val Val Glu Glu Lys
Ala Phe Ser Pro Glu Val Ile Pro Met Phe Ser Ala Leu Ser Glu Gly Ala Thr Pro Gln Asp Leu Asn
Thr Met Leu Asn Thr Val Gly Gly His Gln Ala Ala Met Gln Met Leu Lys Glu Thr Ile Asn Glu
Glu Ala Ala Glu Trp Asp Arg Leu His Pro
(SEQ ID NO: 93)
Leu Ser Pro Arg Thr Leu Asn Ala Trp Val Lys Val Ile Glu Glu Lys
Ala Phe Ser Pro Glu Val Ile Pro Met Phe Thr Ala Leu Ser Glu Gly Ala Thr Pro His Asp Leu Asn
Thr Met Leu Asn Thr Ile Gly Gly His Gln Ala Ala Met Gln Met Leu Lys Asp Thr Ile Asn Glu
Glu Ala Ala Glu Trp Asp Arg Val His Pro
(SEQ ID NO: 94)
Ile Ser Pro Arg Thr Leu Asn Ala Trp Val Lys Val Val Glu Glu Lys
Ala Phe Ser Pro Glu Val Ile Pro Met Phe Ser Ala Leu Ser Glu Gly Ala Thr Pro Gln Asp Leu Asn
Thr Met Leu Asn Thr Val Gly Gly His Gln Ala Ala Met Gln Met Leu Lys Glu Thr Ile Asn Glu
Glu Ala Ala Glu Trp Asp Arg Leu His Pro Val His Ala Gly Pro Ile Ala Pro Gly Gln Met Arg Glu
Pro Arg Gly Ser Asp Ile Ala Gly Thr Thr Ser Thr Leu Gln Glu Gln Ile Gly Trp Met Thr Asn Asn
Pro Pro Ile Pro Val Gly Glu Ile Tyr Lys Arg Trp Ile Ile Leu Gly Leu Asn Lys Ile Val Arg Met Tyr
Ser Pro Thr Ser Ile Leu Asp Ile Arg Gln Gly Pro Lys Glu Pro Phe Arg Asp Tyr Val Asp Arg Phe
Tyr Lys Thr Leu Arg Ala Glu Gln Ala Ser Gln Glu Val Lys Asn Trp Met Thr Glu Thr Leu Leu
Val Gln Asn Ala Asn Pro Asp Cys Lys Thr Ile Leu Lys Ala Leu Gly Pro Ala Ala Thr Leu Glu
Glu Met Met Thr Ala Cys Gln Gly Val Gly Gly Pro Gly His Lys Ala Arg Val Leu Ala Glu Ala
Met Ser Gln
(SEQ ID NO: 95)
Leu Ser Pro Arg Thr Leu Asn Ala Trp Val Lys Val Ile Glu Glu Lys
Ala Phe Ser Pro Glu Val Ile Pro Met Phe Thr Ala Leu Ser Glu Gly Ala Thr Pro His Asp Leu Asn
Thr Met Leu Asn Thr Ile Gly Gly His Gln Ala Ala Met Gln Met Leu Lys Asp Thr Ile Asn Glu
Glu Ala Ala Glu Trp Asp Arg Val His Pro Val His Ala Gly Pro Val Ala Pro Gly Gln Met Arg Asp
Pro Arg Gly Ser Asp Ile Ala Gly Ser Thr Ser Thr Leu Gln Glu Gln Ile Ala Trp Met Thr Asn Asn
Pro Pro Ile Pro Val Gly Asp Ile Tyr Lys Arg Trp Ile Ile Met Gly Leu Asn Lys Ile Val Arg Met
Tyr Ser Pro Val Ser Ile Leu Asp Ile Lys Gln Gly Pro Lys Glu Pro Phe Arg Asp Tyr Val Asp Arg
Phe Tyr Arg Thr Leu Arg Ala Glu Gln Ala Ser Gln Asp Val Lys Asn Trp Met Thr Glu Thr Leu
Leu Val Gln Asn Ser Asn Pro Asp Cys Lys Thr Ile Leu Lys Ala Leu Gly Pro Gly Ala Thr Leu
Glu Glu Met Met Ser Ala Cys Gln Gly Val Gly Gly Pro Ser His Lys Ala Arg Val Leu Ala Glu
Ala Met Cys Gln
(SEQ ID NO: 96)
Arg Thr Leu Asn Ala Trp Val Lys Val
(SEQ ID NO: 97)
Leu Ser Glu Gly Ala Thr Pro Gln Asp Leu Asn Thr Met Leu Asn Thr
Val Gly Gly His Gln Ala Ala Met Gln
(SEQ ID NO: 98)
Leu Ser Glu Gly Ala Thr Pro His Asp Leu Asn Thr Met Leu Asn Thr
Ile Gly Gly His Gln Ala Ala Met Gln
(SEQ ID NO: 99)
Asp Leu Asn Thr Met Leu Asn Thr Val
(SEQ ID NO: 100)
Asp Leu Asn Thr Met Leu Asn Thr Ile
(SEQ ID NO: 101)
Pro Gly Gln Met Arg Glu Pro Arg Gly Ser Asp Ile Ala Gly Thr Thr
Ser Thr Leu Gln Glu Gln Ile Gly Trp Met Thr
(SEQ ID NO: 102)
Pro Gly Gln Met Arg Asp Pro Arg Gly Ser Asp Ile Ala Gly Ser Thr
Ser Thr Leu Gln Glu Gln Ile Ala Trp Met Thr
(SEQ ID NO: 103)
Asn Pro Pro Ile Pro Val Gly Glu Ile Tyr Lys Arg Trp Ile Ile Leu Gly
Leu Asn Lys Ile Val Arg Met Tyr Ser Pro Thr Ser Ile Leu Asp Ile
(SEQ ID NO: 104)
Asn Pro Pro Ile Pro Val Gly Asp Ile Tyr Lys Arg Trp Ile Ile Met Gly
Leu Asn Lys Ile Val Arg Met Tyr Ser Pro Val Ser Ile Leu Asp Ile
(SEQ ID NO: 105)
Pro Val Gly Glu Ile Tyr Lys Arg Trp Ile Ile Leu Gly Leu Asn Lys Ile
Val Arg Met Tyr Ser Pro Thr Ser Ile Leu Asp Ile Arg Gln Gly Pro Lys
(SEQ ID NO: 106)
Pro Val Gly Asp Ile Tyr Lys Arg Trp Ile Ile Met Gly Leu Asn Lys
Ile Val Arg Met Tyr Ser Pro Val Ser Ile Leu Asp Ile Lys Gln Gly Pro Lys
(SEQ ID NO: 107)
Trp Ile Ile Leu Gly Leu Asn Lys Ile Val Arg Met Tyr Ser Pro Thr Ser
Ile
(SEQ ID NO: 108)
Trp Ile Ile Met Gly Leu Asn Lys Ile Val Arg Met Tyr Ser Pro Val
Ser Ile
(SEQ ID NO: 109)
Ser Ile Leu Asp Ile Arg Gln Gly Pro Lys Glu Pro Phe Arg Asp Tyr
Val Asp Arg Phe Tyr Lys Thr Leu Arg Ala Glu Gln Ala Ser Gln Glu Val Lys
(SEQ ID NO: 110)
Ser Ile Leu Asp Ile Lys Gln Gly Pro Lys Glu Pro Phe Arg Asp Tyr
Val Asp Arg Phe Tyr Arg Thr Leu Arg Ala Glu Gln Ala Ser Gln Asp Val Lys
(SEQ ID NO: 111)
Gly Pro Lys Glu Pro Phe Arg Asp Tyr Val Asp Arg Phe Tyr Lys Thr
Leu Arg Ala Glu Gln Ala Ser Gln Glu Val Lys Asn Trp Met Thr Glu Thr Leu
(SEQ ID NO: 112)
Gly Pro Lys Glu Pro Phe Arg Asp Tyr Val Asp Arg Phe Tyr Arg Thr
Leu Arg Ala Glu Gln Ala Ser Gln Asp Val Lys Asn Trp Met Thr Glu Thr Leu
(SEQ ID NO: 113)
Tyr Val Asp Arg Phe Tyr Lys Thr Leu Arg Ala Glu Gln Ala Ser Gln
Glu Val
(SEQ ID NO: 114)
Tyr Val Asp Arg Phe Tyr Arg Thr Leu Arg Ala Glu Gln Ala Ser Gln
Asp Val
(SEQ ID NO: 115)
Gln Glu Val Lys Asn Trp Met Thr Glu Thr Leu Leu Val Gln Asn Ala
Asn Pro Asp Cys Lys Thr Ile Leu Lys Ala Leu Gly Pro Ala Ala Thr Leu Glu Glu Met Met Thr
Ala Cys Gln Gly Val Gly Gly Pro Gly His Lys Ala Arg Val Leu Ala Glu Ala Met Ser Gln
(SEQ ID NO: 116)
Gln Asp Val Lys Asn Trp Met Thr Glu Thr Leu Leu Val Gln Asn Ser
Asn Pro Asp Cys Lys Thr Ile Leu Lys Ala Leu Gly Pro Gly Ala Thr Leu Glu Glu Met Met Ser
Ala Cys Gln Gly Val Gly Gly Pro Ser His Lys Ala Arg Val Leu Ala Glu Ala Met Cys Gln
(SEQ ID NO: 117)
Ile Leu Lys Ala Leu Gly Pro Ala Ala Thr Leu Glu Glu Met Met Thr
Ala Cys Gln Gly Val Gly Gly Pro Gly
(SEQ ID NO: 118)
Ile Leu Lys Ala Leu Gly Pro Gly Ala Thr Leu Glu Glu Met Met Ser
Ala Cys Gln Gly Val Gly Gly Pro Ser
(SEQ ID NO: 119)
Leu Gly Pro Ala Ala Thr Leu Glu Glu Met Met Thr Ala Cys Gln Gly
Val Gly Gly Pro Gly His Lys Ala Arg
(SEQ ID NO: 120)
Leu Gly Pro Gly Ala Thr Leu Glu Glu Met Met Ser Ala Cys Gln Gly
Val Gly Gly Pro Ser His Lys Ala Arg
(SEQ ID NO: 121)
Ala Thr Leu Glu Glu Met Met Thr Ala
(SEQ ID NO: 122)
Ala Thr Leu Glu Glu Met Met Ser Ala
(SEQ ID NO: 123)
Glu Met Met Thr Ala Cys Gln Gly Val
(SEQ ID NO: 124)
Glu Met Met Ser Ala Cys Gln Gly Val
(SEQ ID NO: 125)
Lys Cys Phe Asn Cys Gly Lys Glu Gly His
(SEQ ID NO: 126)
Lys Cys Phe Asn Cys Gly Arg Glu Gly His
(SEQ ID NO: 127)
Ala Arg Asn Cys Arg Ala Pro Arg Lys
(SEQ ID NO: 128)
Ala Lys Asn Cys Arg Ala Pro Arg Lys
(SEQ ID NO: 129)
Ala Arg Asn Cys Arg Ala Pro Arg Lys Lys Gly Cys Trp Lys Cys Gly
Lys Glu Gly His Gln Met Lys Asp Cys Thr Glu Arg Gln Ala Asn Phe Leu Gly Lys Ile Trp Pro
Ser
(SEQ ID NO: 130)
Ala Lys Asn Cys Arg Ala Pro Arg Lys Arg Gly Cys Trp Lys Cys Gly
Arg Glu Gly His Gln Met Lys Asp Cys Asn Glu Arg Gln Ala Asn Phe Leu Gly Lys Val Trp Pro
Ser
(SEQ ID NO: 131)
Asn Cys Arg Ala Pro Arg Lys Lys Gly Cys Trp Lys Cys Gly
(SEQ ID NO: 132)
Asn Cys Arg Ala Pro Arg Lys Arg Gly Cys Trp Lys Cys Gly
(SEQ ID NO: 133)
Gly Cys Trp Lys Cys Gly Lys Glu Gly His Gln Met Lys Asp Cys Thr
Glu Arg Gln
(SEQ ID NO: 134)
Gly Cys Trp Lys Cys Gly Arg Glu Gly His Gln Met Lys Asp Cys
Asn Glu Arg Gln
(SEQ ID NO: 135)
Lys Asp Cys Thr Glu Arg Gln Ala Asn Phe Leu Gly Lys Ile Trp Pro
Ser
(SEQ ID NO: 136)
Lys Asp Cys Asn Glu Arg Gln Ala Asn Phe Leu Gly Lys Val Trp
Pro Ser
(SEQ ID NO: 137)
Arg Gln Ala Asn Phe Leu Gly Lys Ile Trp Pro Ser His Lys Gly Arg
(SEQ ID NO: 138)
Arg Gln Ala Asn Phe Leu Gly Lys Val Trp Pro Ser His Asn Gly Arg
(SEQ ID NO: 139)
Lys Gly Arg Pro Gly Asn Phe Leu Gln Ser Arg Pro
(SEQ ID NO: 140)
Asn Gly Arg Pro Gly Asn Phe Leu Gln Asn Arg Pro
(SEQ ID NO: 141)
Ser Leu Arg Ser Leu Phe Gly Asn Asp Pro
(SEQ ID NO: 142)
Ser Leu Lys Ser Leu Phe Gly Asn Asp Pro
(SEQ ID NO: 143)
Ser Leu Phe Gly Asn Asp Pro Ser Ser
(SEQ ID NO: 144)
Ser Leu Phe Gly Asn Asp Pro Leu Ser
(SEQ ID NO: 145)
Leu Lys His Ile Val Trp Ala Ser Arg Glu Leu Glu Arg Phe Ala Val
Asn Pro Gly Leu Leu Glu Thr Val Ser Gln Asn Tyr Pro Ile Val Gln Asn
(SEQ ID NO: 146)
Leu Lys His Leu Val Trp Ala Ser Arg Glu Leu Glu Arg Phe Ala Leu
Asn Pro Gly Leu Leu Glu Thr Val Ser Gln Asn Phe Pro Ile Val Gln Asn
(SEQ ID NO: 147)
Gly Val Gly Ala Val Ser Arg Asp Leu
(SEQ ID NO: 148)
Gly Val Gly Ala Ala Ser Arg Asp Leu
(SEQ ID NO: 149)
Glu Glu Val Gly Phe Pro Val Arg Pro Gln Val Pro Leu Arg Pro Met
Thr Tyr Lys
(SEQ ID NO: 150)
Glu Glu Val Gly Phe Pro Val Lys Pro Gln Val Pro Leu Arg Pro Met
Thr Phe Lys
(SEQ ID NO: 151)
Glu Glu Val Gly Phe Pro Val Lys Pro Gln Val Pro Leu Arg Pro Met
Thr Phe Lys Gly Ala Leu Asp Leu Ser His Phe Leu Arg Glu Lys Gly Gly Leu Glu Gly
(SEQ ID NO: 152)
Glu Glu Val Gly Phe Pro Val Arg Pro Gln Val Pro Leu Arg Pro Met
Thr Tyr Lys Gly Ala Leu Asp Leu Ser His Phe Leu Lys Glu Lys Gly Gly Leu Glu Gly
(SEQ ID NO: 153)
Tyr Lys Ala Ala Val Asp Leu Ser His Phe Leu Arg Glu Lys Gly Gly
Leu Glu Gly Ala Ala Tyr
(SEQ ID NO: 154)
Tyr Lys Gly Ala Leu Asp Leu Ser His Phe Leu Lys Glu Lys Gly Gly
Leu Glu Gly Ala Ala Tyr
(SEQ ID NO: 155)
Ser His Phe Leu Lys Glu Lys Gly Gly Leu
(SEQ ID NO: 156)
Ser His Phe Leu Arg Glu Lys Gly Gly Leu
(SEQ ID NO: 157)
Leu Lys Glu Lys Gly Gly Leu Glu Gly
(SEQ ID NO: 158)
Leu Arg Glu Lys Gly Gly Leu Glu Gly
(SEQ ID NO: 159)
Thr Gln Gly Tyr Phe Pro Asp Trp Gln Asn Tyr Thr Pro Gly Pro Gly
(SEQ ID NO: 160)
Thr Gln Gly Phe Phe Pro Asp Trp Gln Asn Tyr Thr Pro Glu Pro Gly
(SEQ ID NO: 161)
Thr Gln Gly Phe Phe Pro Asp Trp Gln Asn Tyr Thr Pro Glu Pro Gly
Ile Arg Phe Pro Leu Thr Phe Gly Trp Cys Phe Lys Leu Val Pro Leu
(SEQ ID NO: 162)
Thr Gln Gly Tyr Phe Pro Asp Trp Gln Asn Tyr Thr Pro Gly Pro Gly
Thr Arg Tyr Pro Leu Thr Phe Gly Trp Cys Phe Lys Leu Val Pro Val
(SEQ ID NO: 163)
Glu Pro Gly Ile Arg Phe Pro Leu Thr Phe Gly Trp Cys Phe Lys Leu
Val Pro Leu
(SEQ ID NO: 164)
Gly Pro Gly Thr Arg Tyr Pro Leu Thr Phe Gly Trp Cys Phe Lys Leu
Val Pro Val
(SEQ ID NO: 165)
Gly Pro Gly Ile Arg Tyr Pro Leu Leu Thr Phe Gly Trp Cys Phe Lys
Leu Pro Val Glu Pro Glu Lys Val Glu
(SEQ ID NO: 166)
Arg Tyr Pro Leu Thr Phe Gly Trp Cys
(SEQ ID NO: 167)
Arg Phe Pro Leu Thr Phe Gly Trp Cys
(SEQ ID NO: 168)
Arg Tyr Pro Leu Thr Phe Gly Trp Cys Phe Lys Leu Val Pro Val
(SEQ ID NO: 169)
Arg Phe Pro Leu Thr Phe Gly Trp Cys Phe Lys Leu Val Pro Leu
(SEQ ID NO: 170)
Pro Leu Thr Phe Gly Trp Cys Phe Lys Leu Val Pro Val
(SEQ ID NO: 171)
Pro Leu Cys Phe Gly Trp Cys Phe Lys Leu Val Pro Leu
(SEQ ID NO: 172)
Leu Thr Phe Gly Trp Cys Phe Lys Leu
(SEQ ID NO: 173)
Leu Cys Phe Gly Trp Cys Phe Lys Leu
(SEQ ID NO: 174)
Phe Pro Gln Ile Thr Leu Trp Gln Arg Pro Leu Val
(SEQ ID NO: 175)
Leu Pro Gln Ile Thr Leu Trp Gln Arg Pro Ile Val
(SEQ ID NO: 176)
Phe Pro Gln Ile Thr Leu Trp Gln Arg Pro Leu Val Thr Ile Lys Ile Gly
Gly Gln Leu Lys Glu Ala Leu Leu Asp Thr Gly Ala Asp Asp Thr Val Leu Glu Glu Met Asn Leu
Pro Gly Arg Trp Lys Pro Lys Met Ile Gly Gly Ile Gly Gly Phe Ile Lys Val Arg Gln Tyr Asp Gln
(SEQ ID NO: 177)
Leu Pro Gln Ile Thr Leu Trp Gln Arg Pro Ile Val Thr Ile Lys Ile Gly
Gly Gln Ile Lys Glu Ala Leu Leu Asp Thr Gly Ala Asp Asp Thr Val Leu Glu Asp Met Asn Leu
Pro Gly Lys Trp Lys Pro Lys Met Ile Gly Gly Ile Gly Gly Phe Ile Lys Val Lys Gln Tyr Asp Gln
(SEQ ID NO: 178)
Gly Gly Gln Leu Lys Glu Ala Leu Leu Asp Thr Gly Ala Asp Asp
Thr Val Leu Glu Glu
(SEQ ID NO: 179)
Gly Gly Gln Ile Lys Glu Ala Leu Leu Asp Thr Gly Ala Asp Asp Thr
Val Leu Glu Asp
(SEQ ID NO: 180)
Leu Pro Gly Arg Trp Lys Pro Lys Met Ile Gly Gly Ile Gly Gly Phe
Ile Lys Val Arg Gln Tyr Asp Gln
(SEQ ID NO: 181)
Leu Pro Gly Lys Trp Lys Pro Lys Met Ile Gly Gly Ile Gly Gly Phe
Ile Lys Val Lys Gln Tyr Asp Gln
(SEQ ID NO: 182)
Gly Thr Val Leu Val Gly Pro Thr Pro Val Asn Ile Ile Gly Arg Asn
Leu Leu Thr Gln Ile Gly Cys Thr Leu Asn Phe Pro Ile Ser Pro Ile Glu Thr Val Pro Val Lys Leu
Lys Pro Gly Met Asp Gly Pro Lys Val Lys Gln Trp Pro Leu Thr Glu Glu Lys Ile Lys Ala Leu Val
Glu Ile Cys Thr Glu Met Glu Lys Glu Gly Lys Ile Ser Lys Ile Gly Pro Glu Asn Pro Tyr Asn Thr
Pro Val Phe Ala Ile Lys Lys Lys Asp Ser Thr Lys Trp Arg Lys Leu Val Asp Phe Arg Glu Leu
Asn Lys Arg Thr Gln Asp Phe Trp Glu Val Gln Leu Gly Ile Pro His Pro Ala Gly Leu Lys Lys Lys
Lys Ser
(SEQ ID NO: 183)
Gly Thr Val Leu Ile Gly Pro Thr Pro Val Asn Ile Ile Gly Arg Asn
Leu Leu Thr Gln Leu Gly Cys Thr Leu Asn Phe Pro Ile Ser Pro Ile Asp Thr Val Pro Val Lys Leu
Lys Pro Gly Met Asp Gly Pro Arg Val Lys Gln Trp Pro Leu Thr Glu Glu Lys Ile Lys Ala Leu Ile
Glu Ile Cys Thr Glu Met Glu Lys Glu Gly Lys Ile Ser Arg Ile Gly Pro Glu Asn Pro Tyr Asn Thr
Pro Ile Phe Ala Ile Lys Lys Lys Asp Gly Thr Lys Trp Arg Lys Leu Val Asp Phe Arg Glu Leu Asn
Lys Lys Thr Gln Asp Phe Trp Glu Val Gln Leu Gly Ile Pro His Pro Ser Gly Leu Lys Lys Lys Lys
Ser
(SEQ ID NO: 184)
Gly Thr Val Leu Val Gly Pro Thr Pro Val Asn Ile Ile Gly Arg Asn
Leu Leu Thr Gln Ile Gly Cys Thr Leu Asn Phe Pro Ile Ser Pro Ile Glu Thr Val Pro Val Lys Leu
Lys Pro Gly Met Asp Gly Pro Lys Val Lys Gln Trp Pro Leu Thr Glu Glu Lys Ile Lys Ala Leu Val
Glu Ile Cys Thr Glu Met Glu Lys Glu Gly Lys Ile Ser Lys Ile Gly Pro Glu Asn Pro Tyr Asn Thr
Pro Val Phe Ala Ile Lys Lys Lys Asp Ser Thr Lys Trp Arg Lys Leu Val Asp Phe Arg Glu Leu
Asn Lys Arg Thr Gln Asp Phe Trp Glu Val Gln Leu Gly Ile Pro His Pro Ala Gly Leu Lys Lys Lys
Lys Ser Val Thr Val Leu Asp Val Gly Asp Ala Tyr Phe Ser Val Pro Leu Asp Lys
(SEQ ID NO: 185)
Gly Thr Val Leu Ile Gly Pro Thr Pro Val Asn Ile Ile Gly Arg Asn
Leu Leu Thr Gln Leu Gly Cys Thr Leu Asn Phe Pro Ile Ser Pro Ile Asp Thr Val Pro Val Lys Leu
Lys Pro Gly Met Asp Gly Pro Arg Val Lys Gln Trp Pro Leu Thr Glu Glu Lys Ile Lys Ala Leu Ile
Glu Ile Cys Thr Glu Met Glu Lys Glu Gly Lys Ile Ser Arg Ile Gly Pro Glu Asn Pro Tyr Asn Thr
Pro Ile Phe Ala Ile Lys Lys Lys Asp Gly Thr Lys Trp Arg Lys Leu Val Asp Phe Arg Glu Leu Asn
Lys Lys Thr Gln Asp Phe Trp Glu Val Gln Leu Gly Ile Pro His Pro Ser Gly Leu Lys Lys Lys Lys
Ser Val Thr Ile Leu Asp Val Gly Asp Ala Tyr Phe Ser Ile Pro Leu Asp Lys
(SEQ ID NO: 186)
Gly Thr Val Leu Val Gly Pro Thr Pro Val Asn Ile Ile Gly Arg Asn
Leu Leu Thr Gln Ile Gly Cys Thr Leu Asn Phe Pro Ile Ser Pro Ile Glu Thr Val Pro Val Lys Leu
Lys Pro Gly Met Asp Gly Pro Lys Val Lys Gln Trp Pro Leu Thr Glu Glu Lys Ile Lys Ala Leu Val
Glu Ile Cys Thr Glu Met Glu Lys Glu Gly Lys Ile Ser Lys Ile Gly Pro Glu Asn Pro Tyr Asn Thr
Pro Val Phe Ala Ile Lys Lys Lys Asp Ser Thr Lys Trp Arg Lys Leu Val Asp Phe Arg Glu Leu
Asn Lys Arg Thr Gln Asp Phe Trp Glu Val Gln Leu Gly Ile Pro His Pro Ala Gly Leu Lys Lys Lys
Lys Ser Val Thr Val Leu Asp Val Gly Asp Ala Tyr Phe Ser Val Pro Leu Asp Lys Asp Phe Arg
Lys Tyr Thr Ala Phe Thr Ile Pro Ser
(SEQ ID NO: 187)
Gly Thr Val Leu Ile Gly Pro Thr Pro Val Asn Ile Ile Gly Arg Asn
Leu Leu Thr Gln Leu Gly Cys Thr Leu Asn Phe Pro Ile Ser Pro Ile Asp Thr Val Pro Val Lys Leu
Lys Pro Gly Met Asp Gly Pro Arg Val Lys Gln Trp Pro Leu Thr Glu Glu Lys Ile Lys Ala Leu Ile
Glu Ile Cys Thr Glu Met Glu Lys Glu Gly Lys Ile Ser Arg Ile Gly Pro Glu Asn Pro Tyr Asn Thr
Pro Ile Phe Ala Ile Lys Lys Lys Asp Gly Thr Lys Trp Arg Lys Leu Val Asp Phe Arg Glu Leu Asn
Lys Lys Thr Gln Asp Phe Trp Glu Val Gln Leu Gly Ile Pro His Pro Ser Gly Leu Lys Lys Lys Lys
Ser Val Thr Val Leu Asp Ile Gly Asp Ala Tyr Phe Ser Val Pro Leu Asp Lys Glu Phe Arg Lys Tyr
Thr Ala Phe Thr Val Pro Ser
(SEQ ID NO: 188)
Gly Thr Val Leu Val Gly Pro Thr Pro Val Asn Ile Ile Gly Arg Asn
Leu Leu Thr Gln Ile Gly Cys Thr Leu Asn Phe Pro Ile Ser Pro Ile Glu Thr Val Pro Val Lys Leu
Lys Pro Gly Met Asp Gly Pro Lys Val Lys Gln Trp Pro Leu Thr Glu Glu Lys Ile Lys Ala Leu Val
Glu Ile Cys Thr Glu Met Glu Lys Glu Gly Lys Ile Ser Lys Ile Gly Pro Glu Asn Pro Tyr Asn Thr
Pro Val Phe Ala Ile Lys Lys Lys Asp Ser Thr Lys Trp Arg Lys Leu Val Asp Phe Arg Glu Leu
Asn Lys Arg Thr Gln Asp Phe Trp Glu Val Gln Leu Gly Ile Pro His Pro Ala Gly Leu Lys Lys Lys
Lys Ser Val Thr Val Leu Asp Val Gly Asp Ala Tyr Phe Ser Val Pro Leu Asp Lys Asp Phe Arg
Lys Tyr Thr Ala Phe Thr Ile Pro Ser Ile Asn Asn Glu Thr Pro Gly Ile Arg Tyr Gln Tyr Asn Val
Leu Pro Gln Gly Trp Lys Gly Ser Pro Ala Ile Phe Gln Ser Ser Met Thr
(SEQ ID NO: 189)
Gly Thr Val Leu Ile Gly Pro Thr Pro Val Asn Ile Ile Gly Arg Asn
Leu Leu Thr Gln Leu Gly Cys Thr Leu Asn Phe Pro Ile Ser Pro Ile Asp Thr Val Pro Val Lys Leu
Lys Pro Gly Met Asp Gly Pro Arg Val Lys Gln Trp Pro Leu Thr Glu Glu Lys Ile Lys Ala Leu Ile
Glu Ile Cys Thr Glu Met Glu Lys Glu Gly Lys Ile Ser Arg Ile Gly Pro Glu Asn Pro Tyr Asn Thr
Pro Ile Phe Ala Ile Lys Lys Lys Asp Gly Thr Lys Trp Arg Lys Leu Val Asp Phe Arg Glu Leu Asn
Lys Lys Thr Gln Asp Phe Trp Glu Val Gln Leu Gly Ile Pro His Pro Ser Gly Leu Lys Lys Lys Lys
Ser Val Thr Val Leu Asp Ile Gly Asp Ala Tyr Phe Ser Val Pro Leu Asp Lys Glu Phe Arg Lys Tyr
Thr Ala Phe Thr Val Pro Ser Thr Asn Asn Glu Thr Pro Gly Val Arg Tyr Gln Tyr Asn Val Leu
Pro Met Gly Trp Lys Gly Ser Pro Ala Ile Phe Gln Cys Ser Met Thr
(SEQ ID NO: 190)
Asn Leu Leu Thr Gln Ile Gly Cys Thr Leu Asn Phe Pro Ile Ser Pro
Ile Glu Thr Val Pro Val Lys Leu Lys
(SEQ ID NO: 191)
Asn Leu Leu Thr Gln Leu Gly Cys Thr Leu Asn Phe Pro Ile Ser Pro
Ile Asp Thr Val Pro Val Lys Leu Lys
(SEQ ID NO: 192)
Thr Leu Asn Phe Pro Ile Ser Pro Ile
(SEQ ID NO: 193)
Gly Leu Lys Lys Lys Lys Ser Val Thr Val Leu Asp Val Gly Asp Ala
Tyr Phe Ser Val Pro Leu Asp Lys
(SEQ ID NO: 194)
Gly Leu Lys Lys Asn Lys Ser Val Thr Val Leu Asp Val Gly Asp Ala
Tyr Phe Ser Ile Pro Leu Asp Lys
(SEQ ID NO: 195)
Asp Phe Arg Lys Tyr Thr Ala Phe Thr Ile Pro Ser
(SEQ ID NO: 196)
Glu Phe Arg Lys Tyr Thr Ala Phe Thr Val Pro Ser
(SEQ ID NO: 197)
Asn Asn Glu Thr Pro Gly Ile Arg Tyr Gln Tyr Asn Val Leu Pro Gln
Gly Trp Lys Gly Ser Pro Ala Ile Phe
(SEQ ID NO: 198)
Asn Asn Glu Thr Pro Gly Val Arg Tyr Gln Tyr Asn Val Leu Pro Met
Gly Trp Lys Gly Ser Pro Ala Ile Phe
(SEQ ID NO: 199)
Asn Asn Glu Thr Pro Gly Ile Arg Tyr Gln Tyr Asn Val Leu Pro Gln
Gly Trp Lys Gly Ser Pro Ala Ile Phe Gln Ser Ser Met Thr
(SEQ ID NO: 200)
Asn Asn Glu Thr Pro Gly Val Arg Tyr Gln Tyr Asn Val Leu Pro Met
Gly Trp Lys Gly Ser Pro Ala Ile Phe Gln Cys Ser Met Thr
(SEQ ID NO: 201)
Tyr Gln Tyr Asn Val Leu Pro Gln Gly
(SEQ ID NO: 202)
Tyr Gln Tyr Asn Val Leu Pro Met Gly
(SEQ ID NO: 203)
Phe Gln Ser Ser Met Thr Lys Ile Leu
(SEQ ID NO: 204)
Phe Gln Cys Ser Met Thr Lys Ile Leu
(SEQ ID NO: 205)
Ser Met Thr Lys Ile Leu Glu Pro Phe Arg
(SEQ ID NO: 206)
Ser Met Thr Lys Ile Leu Asp Pro Phe Arg
(SEQ ID NO: 207)
Ile Leu Glu Pro Phe Arg Lys Gln Asn
(SEQ ID NO: 208)
Ile Leu Asp Pro Phe Arg Lys Gln Asn
(SEQ ID NO: 209)
Phe Arg Lys Gln Asn Pro Asp Ile Val Ile Tyr Gln Tyr Met Asp Asp
Leu Tyr Val Gly Ser Asp Leu Glu Ile
(SEQ ID NO: 210)
Lys Gln Asn Pro Asp Ile Val Ile Tyr Gln Tyr Met Asp Asp Leu Tyr
Val Gly Ser Asp Leu Glu Ile Gly Gln
(SEQ ID NO: 211)
Asn Pro Asp Ile Val Ile Tyr Gln Tyr Met Asp Asp Leu Tyr Val Gly
Ser Asp Leu Glu Ile Gly Gln His Arg
(SEQ ID NO: 212)
Asn Pro Asp Ile Val Ile Tyr Gln Tyr Val Asp Asp Leu Tyr Val Gly
Ser Asp Leu Glu Ile Glu Gln His Arg
(SEQ ID NO: 213)
Ile Val Ile Tyr Gln Tyr Met Asp Asp Leu Tyr Val Gly Ser Asp Leu
Glu Ile Gly Gln His Arg
(SEQ ID NO: 214)
Ile Val Ile Tyr Gln Tyr Val Asp Asp Leu Tyr Val Gly Ser Asp Leu
Glu Ile Glu Gln His Arg
(SEQ ID NO: 215)
Val Ile Tyr Gln Tyr Met Asp Asp Leu
(SEQ ID NO: 216)
Val Ile Tyr Gln Tyr Val Asp Asp Leu
(SEQ ID NO: 217)
Tyr Gln Tyr Met Asp Asp Leu Tyr Val
(SEQ ID NO: 218)
Tyr Gln Tyr Val Asp Asp Leu Tyr Val
(SEQ ID NO: 219)
Tyr Met Asp Asp Leu Tyr Val Gly Ser
(SEQ ID NO: 220)
Tyr Val Asp Asp Leu Tyr Val Gly Ser
(SEQ ID NO: 221)
Trp Gly Phe Thr Thr Pro Asp Lys Lys His Gln Lys Glu Pro Pro Phe
Leu Trp Met Gly Tyr Glu Leu His Pro Asp Lys Trp Thr Val Gln Pro Ile
(SEQ ID NO: 222)
Trp Gly Leu Thr Thr Pro Asp Lys Lys His Gln Lys Asp Pro Pro Phe
Leu Trp Met Gly Tyr Glu Leu His Pro Asp Arg Trp Thr Val Gln Pro Ile
(SEQ ID NO: 223)
Trp Gly Phe Thr Thr Pro Asp Lys Lys His Gln Lys Glu Pro Pro Phe
Leu Trp Met Gly Tyr Glu Leu His Pro Asp Lys Trp Thr Val Gln Pro Ile Val Leu Pro Glu Lys Asp
Ser Trp Thr Val Asn Asp Ile Gln Lys Leu Val Gly Lys Leu Asn Trp Ala Ser Gln Ile Tyr Pro Gly
Ile Lys Val
(SEQ ID NO: 224)
Trp Gly Leu Thr Thr Pro Asp Lys Lys His Gln Lys Asp Pro Pro Phe
Leu Trp Met Gly Tyr Glu Leu His Pro Asp Arg Trp Thr Val Gln Pro Ile Glu Leu Pro Glu Lys Glu
Ser Trp Thr Val Asn Asp Ile Gln Lys Leu Ile Gly Lys Leu Asn Trp Ala Ser Gln Ile Tyr Ala Gly
Ile Lys Val
(SEQ ID NO: 225)
Lys Lys His Gln Lys Glu Pro Pro Phe Leu Trp Met Gly Tyr Glu Leu
His Pro Asp Lys Trp Thr Val Gln Pro
(SEQ ID NO: 226)
Lys Lys His Gln Lys Asp Pro Pro Phe Leu Trp Met Gly Tyr Glu Leu
His Pro Asp Arg Trp Thr Val Gln Pro
(SEQ ID NO: 227)
Pro Pro Phe Leu Trp Met Gly Tyr Glu Leu His Pro Asp Lys Trp Thr
Val Gln Pro Ile Val Leu Pro Glu Lys
(SEQ ID NO: 228)
Pro Pro Phe Leu Trp Met Gly Tyr Glu Leu His Pro Asp Arg Trp Thr
Val Gln Pro Ile Glu Leu Pro Glu Lys
(SEQ ID NO: 229)
Phe Leu Trp Met Gly Tyr Glu Leu His
(SEQ ID NO: 230)
Glu Leu His Pro Asp Lys Trp Thr Val
(SEQ ID NO: 231)
Glu Leu His Pro Asp Arg Trp Thr Val
(SEQ ID NO: 232)
Ile Val Leu Pro Glu Lys Asp Ser Trp Thr Val Asn Asp Ile Gln Lys
Leu Val Gly Lys Leu Asn Trp Ala Ser
(SEQ ID NO: 233)
Ile Glu Leu Pro Glu Lys Glu Ser Trp Thr Val Asn Asp Ile Gln Lys
Leu Ile Gly Lys Leu Asn Trp Ala Ser
(SEQ ID NO: 234)
Val Leu Pro Glu Lys Asp Ser Trp Thr Val Asn Asp Ile Gln Lys Leu
Val Gly Lys Leu Asn Trp Ala Ser Gln
(SEQ ID NO: 235)
Glu Leu Pro Glu Lys Glu Ser Trp Thr Val Asn Asp Ile Gln Lys Leu
Ile Gly Lys Leu Asn Trp Ala Ser Gln
(SEQ ID NO: 236)
Leu Pro Glu Lys Asp Ser Trp Thr Val Asn Asp Ile Gln Lys Leu Val
Gly Lys Leu Asn Trp Ala Ser Gln Ile Tyr Pro Gly Ile Lys Val
(SEQ ID NO: 237)
Leu Pro Glu Lys Glu Ser Trp Thr Val Asn Asp Ile Gln Lys Leu Ile
Gly Lys Leu Asn Trp Ala Ser Gln Ile Tyr Ala Gly Ile Lys Val
(SEQ ID NO: 238)
Ser Trp Thr Val Asn Asp Ile Gln Lys Leu Val Gly Lys Leu Asn Trp
Ala Ser Gln Ile Tyr Pro Gly Ile Lys
(SEQ ID NO: 239)
Ser Trp Thr Val Asn Asp Ile Gln Lys Leu Ile Gly Lys Leu Asn Trp
Ala Ser Gln Ile Tyr Ala Gly Ile Lys
(SEQ ID NO: 240)
Trp Thr Val Asn Asp Ile Gln Lys Leu
(SEQ ID NO: 241)
Thr Val Asn Asp Ile Gln Lys Leu Val
(SEQ ID NO: 242)
Thr Val Asn Asp Ile Gln Lys Leu Ile
(SEQ ID NO: 243)
Lys Leu Val Gly Lys Leu Asn Trp Ala
(SEQ ID NO: 244)
Lys Leu Ile Gly Lys Leu Asn Trp Ala
(SEQ ID NO: 245)
Leu Cys Lys Leu Leu Arg Gly Thr Lys
(SEQ ID NO: 246)
Leu Cys Lys Leu Leu Arg Gly Ala Lys
(SEQ ID NO: 247)
Glu Ala Glu Leu Glu Leu Ala Glu Asn Arg Glu Ile Leu Lys Glu Pro
Val His Gly
(SEQ ID NO: 248)
Glu Ala Glu Ile Glu Leu Ala Glu Asn Arg Glu Ile Leu Arg Glu Pro
Val His Gly
(SEQ ID NO: 249)
Glu Pro Val His Gly Val Tyr Tyr Asp Pro Ser Lys
(SEQ ID NO: 250)
Glu Pro Val His Gly Ala Tyr Tyr Asp Pro Ser Lys
(SEQ ID NO: 251)
Gly Gln Trp Thr Tyr Gln Ile Tyr Gln Glu Pro Phe Lys Asn Leu Lys
Thr Gly Lys Tyr Ala Arg
(SEQ ID NO: 252)
Gly Gln Trp Ser Tyr Gln Ile Tyr Gln Glu Pro Tyr Lys Asn Leu Lys
Thr Gly Lys Tyr Ala Lys
(SEQ ID NO: 253)
Ala His Thr Asn Asp Val Lys Gln Leu Thr Glu Ala Val Gln Lys Ile
(SEQ ID NO: 254)
Ala His Thr Asn Asp Val Arg Gln Leu Thr Glu Ala Val Gln Lys Val
(SEQ ID NO: 255)
Ile Val Ile Trp Gly Lys Thr Pro Lys Phe
(SEQ ID NO: 256)
Ile Val Ile Trp Gly Lys Ile Pro Lys Phe
(SEQ ID NO: 257)
Pro Lys Phe Lys Leu Pro Ile Gln Lys Glu Thr Trp Glu
(SEQ ID NO: 258)
Pro Lys Phe Arg Leu Pro Ile Gln Lys Glu Thr Trp Asp
(SEQ ID NO: 259)
Pro Lys Phe Lys Leu Pro Ile Gln Lys Glu Thr Trp Glu Thr Trp Trp
Thr Glu Tyr Trp Gln Ala Thr Trp Ile Pro Glu Trp Glu Phe Val Asn Thr Pro Pro Leu Val Lys Leu
Trp Tyr Gln Leu Glu Lys Glu Pro Ile Val Gly Ala Glu Thr Phe Tyr Val Asp Gly Ala Ala Asn Arg
Glu Thr Lys
(SEQ ID NO: 260)
Pro Lys Phe Arg Leu Pro Ile Gln Lys Glu Thr Trp Asp Thr Trp Trp
Thr Asp Tyr Trp Gln Ala Thr Trp Ile Pro Glu Trp Glu Phe Thr Asn Thr Pro Pro Leu Val Lys Leu
Trp Tyr Gln Leu Glu Thr Glu Pro Ile Ala Gly Val Glu Thr Phe Tyr Val Asp Gly Ala Ser Asn Arg
Glu Thr Lys
(SEQ ID NO: 261)
Trp Glu Thr Trp Trp Thr Glu Tyr Trp Gln Ala Thr Trp Ile Pro Glu
Trp Glu Phe Val Asn Thr Pro Pro Leu
(SEQ ID NO: 262)
Trp Asp Thr Trp Trp Thr Asp Tyr Trp Gln Ala Thr Trp Ile Pro Glu
Trp Glu Phe Thr Asn Thr Pro Pro Leu
(SEQ ID NO: 263)
Glu Tyr Trp Gln Ala Thr Trp Ile Pro Glu Trp Glu Phe Val Asn Thr
Pro Pro Leu Val Lys Leu Trp Tyr Gln Leu Glu Lys Glu Pro Ile
(SEQ ID NO: 264)
Asp Tyr Trp Gln Ala Thr Trp Ile Pro Glu Trp Glu Phe Thr Asn Thr
Pro Pro Leu Val Lys Leu Trp Tyr Gln Leu Glu Thr Glu Pro Ile
(SEQ ID NO: 265)
Trp Gln Ala Thr Trp Ile Pro Glu Trp
(SEQ ID NO: 266)
Gly Ala Glu Thr Phe Tyr Val Asp Gly Ala Ala Asn Arg Glu Thr Lys
(SEQ ID NO: 267)
Gly Val Glu Thr Phe Tyr Val Asp Gly Ala Ser Asn Arg Glu Thr Lys
(SEQ ID NO: 268)
Thr Asp Thr Thr Asn Gln Lys Thr Glu Leu Gln Ala Ile
(SEQ ID NO: 269)
Ala Asp Thr Thr Asn Gln Lys Thr Glu Leu His Ala Ile
(SEQ ID NO: 270)
Ala Ile His Leu Ala Leu Gln Asp Ser
(SEQ ID NO: 271)
Ala Ile Tyr Leu Ala Leu Gln Asp Ser
(SEQ ID NO: 272)
Leu Ala Leu Gln Asp Ser Gly Leu Glu Val Asn Ile Val Thr Asp Ser
Gln Tyr Ala Leu Gly Ile Ile Gln Ala Gln Pro Asp Lys Ser Glu Ser Glu
(SEQ ID NO: 273)
Leu Ala Leu Gln Asp Ser Gly Ser Glu Val Asn Ile Val Thr Asp Ser
Gln Tyr Ala Ile Gly Ile Ile Gln Ala Gln Pro Asp Arg Ser Glu Ser Glu
(SEQ ID NO: 274)
Gln Asp Ser Gly Leu Glu Val Asn Ile Val Thr Asp Ser Gln Tyr Ala
Leu Gly Ile Ile Gln Ala Gln Pro Asp
(SEQ ID NO: 275)
Gln Asp Ser Gly Ser Glu Val Asn Ile Val Thr Asp Ser Gln Tyr Ala
Ile Gly Ile Ile Gln Ala Gln Pro Asp
(SEQ ID NO: 276)
Ile Val Thr Asp Ser Gln Tyr Ala Leu
(SEQ ID NO: 277)
Ile Val Thr Asp Ser Gln Tyr Ala Ile
(SEQ ID NO: 278)
Val Ser Gln Ile Ile Glu Gln Leu Ile
(SEQ ID NO: 279)
Val Asn Gln Ile Ile Glu Gln Leu Ile
(SEQ ID NO: 280)
Gln Ile Ile Glu Gln Leu Ile Lys Lys Glu
(SEQ ID NO: 281)
Gln Ile Ile Glu Gln Leu Ile Asn Lys Glu
(SEQ ID NO: 282)
Lys Glu Lys Val Tyr Leu Ala Trp Val Pro Ala His Lys Gly Ile Gly
Gly Asn Glu Gln Val Asp Lys Leu Val Ser
(SEQ ID NO: 283)
Lys Glu Lys Ile Tyr Leu Ala Trp Val Pro Ala His Lys Gly Ile Gly
Gly Asn Glu Gln Ile Asp Lys Leu Val Ser
(SEQ ID NO: 284)
Gly Ile Arg Lys Val Leu Phe Leu Asp Gly Ile Asp Lys Ala Gln Glu
(SEQ ID NO: 285)
Gly Ile Arg Arg Val Leu Phe Leu Asp Gly Ile Glu Lys Ala Gln Asp
(SEQ ID NO: 286)
His Glu Lys Tyr His Ser Asn Trp Arg
(SEQ ID NO: 287)
His Glu Lys Tyr His Asn Asn Trp Arg
(SEQ ID NO: 288)
Met Ala Ser Asp Phe Asn Leu Pro Pro
(SEQ ID NO: 289)
Met Ala Ser Asp Phe Asn Ile Pro Pro
(SEQ ID NO: 290)
Phe Asn Leu Pro Pro Val Val Ala Lys Glu Ile Val Ala
(SEQ ID NO: 291)
Phe Asn Leu Pro Pro Ile Val Ala Lys Glu Ile Val Ala
(SEQ ID NO: 292)
Phe Asn Leu Pro Pro Val Val Ala Lys Glu Ile Val Ala Ser Cys Asp
Lys Cys Gln Leu Lys Gly Glu Ala Met His Gly Gln Val Asp Cys Ser Pro Gly Ile Trp Gln Leu
Asp Cys Thr His Leu Glu Gly Lys Ile Ile Leu Val Ala Val His Val Ala Ser Gly Tyr Ile Glu Ala
Glu Val Ile Pro Ala Glu Thr Gly Gln Glu Thr Ala Tyr Phe Leu Leu Lys Leu Ala Gly Arg Trp Pro
Val Lys Thr
(SEQ ID NO: 293)
Phe Asn Leu Pro Pro Ile Val Ala Lys Glu Ile Val Ala Cys Cys Asp
Lys Cys Gln Leu Lys Gly Glu Ala Ile His Gly Gln Val Asp Cys Ser Pro Gly Val Trp Gln Leu Asp
Cys Thr His Leu Glu Gly Lys Val Ile Leu Val Ala Val His Val Ala Ser Gly Tyr Ile Glu Ala Glu
Ile Ile Pro Thr Glu Thr Gly Gln Glu Thr Ala Tyr Phe Ile Leu Lys Leu Ala Gly Arg Trp Pro Val
Thr Thr
(SEQ ID NO: 294)
Val Ala Lys Glu Ile Val Ala Ser Cys Asp Lys Cys Gln Leu Lys Gly
Glu Ala Met His Gly Gln Val Asp Cys Ser Pro Gly Ile Trp Gln Leu Asp Cys Thr His Leu Glu Gly
Lys Ile Ile Leu Val Ala Val His Val Ala Ser Gly Tyr Ile Glu Ala Glu Val Ile Pro Ala Glu Thr Gly
Gln Glu Thr Ala Tyr Phe Leu Leu Lys Leu Ala Gly Arg Trp Pro Val Lys Thr
(SEQ ID NO: 295)
Val Ala Lys Glu Ile Val Ala Cys Cys Asp Lys Cys Gln Leu Lys Gly
Glu Ala Ile His Gly Gln Val Asp Cys Ser Pro Gly Val Trp Gln Leu Asp Cys Thr His Leu Glu Gly
Lys Val Ile Leu Val Ala Val His Val Ala Ser Gly Tyr Met Glu Ala Glu Val Ile Pro Thr Glu Thr
Gly Gln Glu Thr Ala Tyr Phe Ile Leu Lys Leu Ala Gly Arg Trp Pro Val Thr Thr
(SEQ ID NO: 296)
Gln Leu Lys Gly Glu Ala Met His Gly Gln Val Asp Cys Ser Pro Gly
Ile Trp Gln Leu Asp Cys Thr His Leu
(SEQ ID NO: 297)
Gln Leu Lys Gly Glu Ala Ile His Gly Gln Val Asp Cys Ser Pro Gly
Val Trp Gln Leu Asp Cys Thr His Leu
(SEQ ID NO: 298)
Gly Gln Val Asp Cys Ser Pro Gly Ile
(SEQ ID NO: 299)
Gly Gln Val Asp Cys Ser Pro Gly Val
(SEQ ID NO: 300)
Gln Val Asp Cys Ser Pro Gly Ile Trp Gln Leu Asp Cys Thr His Leu
Glu Gly Lys Ile Ile Leu Val Ala Val
(SEQ ID NO: 301)
Gln Val Asp Cys Ser Pro Gly Val Trp Gln Leu Asp Cys Thr His Leu
Glu Gly Lys Val Ile Leu Val Ala Val
(SEQ ID NO: 302)
Trp Gln Leu Asp Cys Thr His Leu Glu
(SEQ ID NO: 303)
Ser Asn Phe Thr Ser Thr Thr Val Lys Ala Ala Cys Trp Trp Ala Gly
Ile Lys Gln Glu Phe Gly Ile Pro Tyr
(SEQ ID NO: 304)
Ser Asn Phe Thr Ser Thr Ala Val Lys Ala Ala Cys Trp Trp Ala Gly
Val Lys Gln Glu Phe Gly Ile Pro Tyr
(SEQ ID NO: 305)
Thr Val Lys Ala Ala Cys Trp Trp Ala Gly Ile Lys Gln Glu Phe Gly
Ile Pro Tyr Asn Pro Gln Ser Gln Gly Val Val Glu Ser Met Asn Lys Glu Leu Lys Lys Ile Ile Gly
Gln Val Arg Asp Gln Ala Glu His Leu Lys Thr Ala Val Gln Met Ala Val Phe Ile His Asn Phe Lys
Arg Lys Gly Gly Ile Gly Gly Tyr Ser Ala Gly Glu Arg Ile Val Asp Ile Ile
(SEQ ID NO: 306)
Ala Val Lys Ala Ala Cys Trp Trp Ala Gly Val Lys Gln Glu Phe Gly
Ile Pro Tyr His Pro Gln Ser Gln Gly Val Val Glu Ser Met Asn Asn Glu Leu Lys Lys Ile Ile Gly
Gln Ile Arg Asp Gln Ala Glu Gln Leu Lys Thr Ala Val Gln Met Ala Val Leu Ile His Asn Phe Lys
Arg Lys Gly Gly Ile Gly Glu Tyr Ser Ala Gly Glu Arg Ile Ile Asp Ile Ile
(SEQ ID NO: 307)
Thr Val Lys Ala Ala Cys Trp Trp Ala Gly Ile Lys Gln Glu Phe Gly
Ile Pro Tyr Asn Pro Gln Ser Gln Gly Val Val Glu Ser Met Asn Lys Glu Leu Lys Lys Ile Ile Gly
Gln Val Arg Asp Gln Ala Glu His Leu Lys Thr Ala Val Gln Met Ala Val Phe Ile His Asn Phe Lys
Arg Lys Gly Gly Ile Gly Gly Tyr Ser Ala Gly Glu Arg Ile Val Asp Ile Ile Ala
(SEQ ID NO: 308)
Ala Val Lys Ala Ala Cys Trp Trp Ala Gly Val Lys Gln Glu Phe Gly
Ile Pro Tyr His Pro Gln Ser Gln Gly Val Val Glu Ser Met Asn Asn Glu Leu Lys Lys Ile Ile Gly
Gln Ile Arg Asp Gln Ala Glu Gln Leu Lys Thr Ala Val Gln Met Ala Val Leu Ile His Asn Phe Lys
Arg Lys Gly Gly Ile Gly Glu Tyr Ser Ala Gly Glu Arg Ile Ile Asp Ile Ile Ala
(SEQ ID NO: 309)
Thr Val Lys Ala Ala Cys Trp Trp Ala Gly Ile Lys Gln Glu Phe Gly
Ile Pro Tyr Asn Pro Gln Ser Gln Gly Val Val Glu Ser Met Asn Lys Glu Leu Lys Lys Ile Ile Gly
Gln Val Arg Asp Gln Ala Glu His Leu Lys Thr Ala Val Gln Met Ala Val Phe Ile His Asn Phe Lys
Arg Lys Gly Gly Ile Gly Gly Tyr Ser Ala Gly Glu Arg Ile Val Asp Ile Ile Ala Thr Asp Ile Gln Thr
Lys Glu Leu Gln Lys Gln Ile Thr Lys Ile Gln Asn Phe Arg Val Tyr Tyr Arg Asp Ser Arg Asp Pro
Leu Trp Lys Gly Pro Ala Lys Leu Leu Trp Lys Gly Glu Gly Ala Val Val Ile Gln Asp Asn Ser Asp
Ile Lys Val Val Pro Arg Arg Lys Ala Lys Ile Ile Arg Asp Tyr Gly Lys Gln Met Ala Gly Asp Asp
Cys Val Ala Ser Arg Gln Asp Glu Asp
(SEQ ID NO: 310)
Ala Val Lys Ala Ala Cys Trp Trp Ala Gly Val Lys Gln Glu Phe Gly
Ile Pro Tyr Asn Thr Gln Ser Gln Gly Val Val Glu Ser Met Asn Asn Glu Leu Lys Lys Ile Ile Gly
Gln Ile Arg Asp Gln Ala Glu His Leu Lys Thr Ala Val Gln Met Ala Val Leu Ile His Asn Phe Lys
Arg Lys Gly Gly Ile Gly Glu Tyr Ser Ala Gly Glu Arg Ile Ile Asp Ile Ile Ala Thr Asp Ile Gln Thr
Arg Glu Leu Gln Lys Gln Ile Thr Lys Leu Gln Asn Phe Arg Val Tyr Tyr Arg Asp Asn Arg Asp
Pro Leu Trp Lys Gly Pro Ala Arg Leu Leu Trp Lys Gly Glu Gly Ala Val Val Ile Gln Asp Asn Ser
Glu Ile Lys Val Val Pro Arg Arg Lys Val Lys Ile Ile Arg Asp Tyr Gly Lys Arg Met Ala Gly Asp
Asp Cys Val Ala Gly Arg Gln Asp Glu Asp
(SEQ ID NO: 311)
Lys Ala Ala Cys Trp Trp Ala Gly Ile
(SEQ ID NO: 312)
Lys Ala Ala Cys Trp Trp Ala Gly Val
(SEQ ID NO: 313)
Asp Ile Ile Ala Thr Asp Ile Gln Thr
(SEQ ID NO: 314)
Asp Ile Ile Ala Ser Asp Ile Gln Thr
(SEQ ID NO: 315)
Asp Ile Gln Thr Lys Glu Leu Gln Lys
(SEQ ID NO: 316)
Asp Ile Gln Thr Arg Glu Leu Gln Lys
(SEQ ID NO: 317)
Gln Thr Lys Glu Leu Gln Lys Gln Ile
(SEQ ID NO: 318)
Gln Thr Arg Glu Leu Gln Lys Gln Ile
(SEQ ID NO: 319)
Ala Ile Thr Lys Ile Gln Asn Phe Arg Val Tyr Tyr Arg Asp Ser Arg
Asp Pro Leu Trp Lys Gly Pro Ala Lys Leu Leu Trp Lys Gly Glu Gly Ala Val Val Ile Gln Asp
Asn Ser Asp Ile Lys Val Val Pro Arg Arg Lys Ala Lys Ile Ile Arg Asp Tyr Gly Lys Gln Met Ala
Gly Asp Asp Cys Val Ala Ser Arg Gln Asp Glu Asp
(SEQ ID NO: 320)
Ala Ile Thr Lys Leu Gln Asn Phe Arg Val Tyr Tyr Arg Asp Asn Arg
Asp Pro Leu Trp Lys Gly Pro Ala Arg Leu Leu Trp Lys Gly Glu Gly Ala Val Val Ile Gln Asp
Asn Ser Glu Ile Lys Val Val Pro Arg Arg Lys Val Lys Ile Ile Arg Asp Tyr Gly Lys Arg Met Ala
Gly Asp Asp Cys Val Ala Gly Arg Gln Asp Glu Asp
(SEQ ID NO: 321)
Ile Thr Lys Ile Gln Asn Phe Arg Val Tyr Tyr Arg Asp Ser Arg Asp
Pro Leu Trp Lys Gly Pro Ala Lys Leu Leu Trp Lys Gly Glu Gly Ala Val Val Ile Gln Asp Asn Ser
Asp Ile Lys Val Val Pro Arg Arg Lys Ala Lys Ile Ile Arg Asp Tyr Gly Lys Gln Met Ala Gly Asp
Asp Cys Val Ala Ser Arg Gln Asp Glu Asp
(SEQ ID NO: 322)
Ile Thr Lys Leu Gln Asn Phe Arg Val Tyr Tyr Arg Asp Asn Arg Asp
Pro Leu Trp Lys Gly Pro Ala Arg Leu Leu Trp Lys Gly Glu Gly Ala Val Val Ile Gln Asp Asn Ser
Glu Ile Lys Val Val Pro Arg Arg Lys Val Lys Ile Ile Arg Asp Tyr Gly Lys Arg Met Ala Gly Asp
Asp Cys Val Ala Gly Arg Gln Asp Glu Asp
(SEQ ID NO: 323)
Val Tyr Tyr Arg Asp Ser Arg Asp Pro Leu Trp Lys Gly Pro Ala Lys
Leu Leu Trp Lys Gly Glu Gly Ala Val
(SEQ ID NO: 324)
Val Tyr Tyr Arg Asp Asn Arg Asp Pro Leu Trp Lys Gly Pro Ala Arg
Leu Leu Trp Lys Gly Glu Gly Ala Val
(SEQ ID NO: 325)
Asp Pro Leu Trp Lys Gly Pro Ala Lys Leu Leu Trp Lys Gly Glu Gly
Ala Val Val Ile Gln Asp Asn Ser Asp
(SEQ ID NO: 326)
Asp Pro Leu Trp Lys Gly Pro Ala Arg Leu Leu Trp Lys Gly Glu Gly
Ala Val Val Ile Gln Asp Asn Ser Glu
(SEQ ID NO: 327)
Pro Leu Trp Lys Gly Pro Ala Lys Leu
(SEQ ID NO: 328)
Pro Leu Trp Lys Gly Pro Ala Arg Leu
(SEQ ID NO: 329)
Pro Leu Trp Lys Gly Pro Ala Lys Leu Leu Trp Lys Gly Glu Gly Ala
Val Val Ile Gln Asp Asn Ser Asp Ile
(SEQ ID NO: 330)
Pro Leu Trp Lys Gly Pro Ala Arg Leu Leu Trp Lys Gly Glu Gly Ala
Val Val Ile Gln Asp Asn Ser Glu Ile
(SEQ ID NO: 331)
Lys Leu Leu Trp Lys Gly Glu Gly Ala
(SEQ ID NO: 332)
Arg Leu Leu Trp Lys Gly Glu Gly Ala
(SEQ ID NO: 333)
Leu Leu Trp Lys Gly Glu Gly Ala Val
(SEQ ID NO: 334)
Ala Lys Ile Ile Arg Asp Tyr Gly Lys Gln Met Ala Gly Asp Asp Cys
Val Ala Ser Arg Gln Asp Glu Asp
(SEQ ID NO: 335)
Val Lys Ile Ile Arg Asp Tyr Gly Lys Arg Met Ala Gly Asp Asp Cys
Val Ala Gly Arg Gln Asp Glu Asp
(SEQ ID NO: 336)
Lys Gln Met Ala Gly Asp Asp Cys Val
(SEQ ID NO: 337)
Lys Arg Met Ala Gly Asp Asp Cys Val
(SEQ ID NO: 338)
Tyr Leu Arg Asp Gln Gln Leu Leu Gly Leu Trp Gly Cys Ser Gly Lys
Leu Ile Cys Pro Thr Ala Val Pro Trp
(SEQ ID NO: 339)
Pro Val Gly Glu Ile Tyr Lys Arg Trp Ile Ile Leu Gly Leu Asn Lys Ile
Val Arg Met Tyr Ser Pro Thr Ser Ile
(SEQ ID NO: 340)
Pro Val Gly Asp Ile Tyr Lys Arg Trp Ile Ile Met Gly Leu Asn Lys
Ile Val Arg Met Tyr Ser Pro Val Ser Ile
(SEQ ID NO: 341)
Gly Pro Lys Glu Pro Phe Arg Asp Tyr Val Asp Arg Phe Tyr Lys Thr
Leu Arg Ala Glu Gln Ala Ser Gln Glu Val
(SEQ ID NO: 342)
Gly Pro Lys Glu Pro Phe Arg Asp Tyr Val Asp Arg Phe Tyr Arg Thr
Leu Arg Ala Glu Gln Ala Ser Gln Asp Val
(SEQ ID NO: 343)
Phe Arg Lys Gln Asn Pro Asp Ile Val Ile Tyr Gln Tyr Val Asp Asp
Leu Tyr Val Gly Ser Asp Leu Glu Ile
(SEQ ID NO: 344)
Lys Gln Asn Pro Asp Ile Val Ile Tyr Gln Tyr Val Asp Asp Leu Tyr
Val Gly Ser Asp Leu Glu Ile Glu Gln
(SEQ ID NO: 345)
Leu Lys His Ile Val Trp Ala Ser Arg Glu Leu Glu Arg Phe Ala Val
Asn Pro Gly Leu Leu Glu Thr Ala Ala Ala Val Ser Gln Asn Tyr Pro Ile Val Gln Asn Ala Ala Ala
Ile Ser Pro Arg Thr Leu Asn Ala Trp Val Lys Val Val Glu Glu Lys Ala Phe Ser Pro Glu Val Ile
Pro Met Phe Ser Ala Leu Ser Glu Gly Ala Thr Pro Gln Asp Leu Asn Thr Met Leu Asn Thr Val
Gly Gly His Gln Ala Ala Met Gln Met Leu Lys Glu Thr Ile Asn Glu Glu Ala Ala Glu Trp Asp
Arg Leu His Pro Val His Ala Gly Pro Ile Ala Pro Gly Gln Met Arg Glu Pro Arg Gly Ser Asp Ile
Ala Gly Thr Thr Ser Thr Leu Gln Glu Gln Ile Gly Trp Met Thr Asn Asn Pro Pro Ile Pro Val Gly
Glu Ile Tyr Lys Arg Trp Ile Ile Leu Gly Leu Asn Lys Ile Val Arg Met Tyr Ser Pro Thr Ser Ile Leu
Asp Ile Arg Gln Gly Pro Lys Glu Pro Phe Arg Asp Tyr Val Asp Arg Phe Tyr Lys Thr Leu Arg
Ala Glu Gln Ala Ser Gln Glu Val Lys Asn Trp Met Thr Glu Thr Leu Leu Val Gln Asn Ala Asn
Pro Asp Cys Lys Thr Ile Leu Lys Ala Leu Gly Pro Ala Ala Thr Leu Glu Glu Met Met Thr Ala
Cys Gln Gly Val Gly Gly Pro Gly His Lys Ala Arg Val Leu Ala Glu Ala Met Ser Gln Ala Ala
Ala Leu Pro Gly Arg Trp Lys Pro Lys Met Ile Gly Gly Ile Gly Gly Phe Ile Lys Val Arg Gln Tyr
Asp Gln Ala Ala Ala Gly Thr Val Leu Val Gly Pro Thr Pro Val Asn Ile Ile Gly Arg Asn Leu Leu
Thr Gln Ile Gly Cys Thr Leu Asn Phe Pro Ile Ser Pro Ile Glu Thr Val Pro Val Lys Leu Lys Pro
Gly Met Asp Gly Pro Lys Val Lys Gln Trp Pro Leu Thr Glu Glu Lys Ile Lys Ala Leu Val Glu Ile
Cys Thr Glu Met Glu Lys Glu Gly Lys Ile Ser Lys Ile Gly Pro Glu Asn Pro Tyr Asn Thr Pro Val
Phe Ala Ile Lys Lys Lys Asp Ser Thr Lys Trp Arg Lys Leu Val Asp Phe Arg Glu Leu Asn Lys
Arg Thr Gln Asp Phe Trp Glu Val Gln Leu Gly Ile Pro His Pro Ala Gly Leu Lys Lys Lys Lys Ser
Val Thr Val Leu Asp Val Gly Asp Ala Tyr Phe Ser Val Pro Leu Asp Lys Asp Phe Arg Lys Tyr
Thr Ala Phe Thr Ile Pro Ser Ala Ala Ala Trp Gly Phe Thr Thr Pro Asp Lys Lys His Gln Lys Glu
Pro Pro Phe Leu Trp Met Gly Tyr Glu Leu His Pro Asp Lys Trp Thr Val Gln Pro Ile Ala Ala Ala
Val Ala Lys Glu Ile Val Ala Ser Cys Asp Lys Cys Gln Leu Lys Gly Glu Ala Met His Gly Gln Val
Asp Cys Ser Pro Gly Ile Trp Gln Leu Asp Cys Thr His Leu Glu Gly Lys Ile Ile Leu Val Ala Val
His Val Ala Ser Gly Tyr Ile Glu Ala Glu Val Ile Pro Ala Glu Thr Gly Gln Glu Thr Ala Tyr Phe
Leu Leu Lys Leu Ala Gly Arg Trp Pro Val Lys Thr Ala Ala Ala Thr Val Lys Ala Ala Cys Trp
Trp Ala Gly Ile Lys Gln Glu Phe Gly Ile Pro Tyr Asn Pro Gln Ser Gln Gly Val Val Glu Ser Met
Asn Lys Glu Leu Lys Lys Ile Ile Gly Gln Val Arg Asp Gln Ala Glu His Leu Lys Thr Ala Val Gln
Met Ala Val Phe Ile His Asn Phe Lys Arg Lys Gly Gly Ile Gly Gly Tyr Ser Ala Gly Glu Arg Ile
Val Asp Ile Ile Ala Ala Ala Ala Ile Thr Lys Ile Gln Asn Phe Arg Val Tyr Tyr Arg Asp Ser Arg
Asp Pro Leu Trp Lys Gly Pro Ala Lys Leu Leu Trp Lys Gly Glu Gly Ala Val Val Ile Gln Asp
Asn Ser Asp Ile Lys Val Val Pro Arg Arg Lys Ala Lys Ile Ile Arg Asp Tyr Gly Lys Gln Met Ala
Gly Asp Asp Cys Val Ala Ser Arg Gln Asp Glu Asp Ala Ala Ala Glu Glu Val Gly Phe Pro Val
Lys Pro Gln Val Pro Leu Arg Pro Met Thr Phe Lys Gly Ala Leu Asp Leu Ser His Phe Leu Arg
Glu Lys Gly Gly Leu Glu Gly
(SEQ ID NO: 346)
Leu Lys His Leu Val Trp Ala Ser Arg Glu Leu Glu Arg Phe Ala Leu
Asn Pro Gly Leu Leu Glu Thr Ala Ala Ala Val Ser Gln Asn Phe Pro Ile Val Gln Asn Ala Ala Ala
Leu Ser Pro Arg Thr Leu Asn Ala Trp Val Lys Val Ile Glu Glu Lys Ala Phe Ser Pro Glu Val Ile
Pro Met Phe Thr Ala Leu Ser Glu Gly Ala Thr Pro His Asp Leu Asn Thr Met Leu Asn Thr Ile
Gly Gly His Gln Ala Ala Met Gln Met Leu Lys Asp Thr Ile Asn Glu Glu Ala Ala Glu Trp Asp
Arg Val His Pro Val His Ala Gly Pro Val Ala Pro Gly Gln Met Arg Asp Pro Arg Gly Ser Asp Ile
Ala Gly Ser Thr Ser Thr Leu Gln Glu Gln Ile Ala Trp Met Thr Asn Asn Pro Pro Ile Pro Val Gly
Asp Ile Tyr Lys Arg Trp Ile Ile Met Gly Leu Asn Lys Ile Val Arg Met Tyr Ser Pro Val Ser Ile
Leu Asp Ile Lys Gln Gly Pro Lys Glu Pro Phe Arg Asp Tyr Val Asp Arg Phe Tyr Arg Thr Leu
Arg Ala Glu Gln Ala Ser Gln Asp Val Lys Asn Trp Met Thr Glu Thr Leu Leu Val Gln Asn Ser
Asn Pro Asp Cys Lys Thr Ile Leu Lys Ala Leu Gly Pro Gly Ala Thr Leu Glu Glu Met Met Ser
Ala Cys Gln Gly Val Gly Gly Pro Ser His Lys Ala Arg Val Leu Ala Glu Ala Met Cys Gln Ala
Ala Ala Leu Pro Gly Lys Trp Lys Pro Lys Met Ile Gly Gly Ile Gly Gly Phe Ile Lys Val Lys Gln
Tyr Asp Gln Ala Ala Ala Gly Thr Val Leu Ile Gly Pro Thr Pro Val Asn Ile Ile Gly Arg Asn Leu
Leu Thr Gln Leu Gly Cys Thr Leu Asn Phe Pro Ile Ser Pro Ile Asp Thr Val Pro Val Lys Leu Lys
Pro Gly Met Asp Gly Pro Arg Val Lys Gln Trp Pro Leu Thr Glu Glu Lys Ile Lys Ala Leu Ile Glu
Ile Cys Thr Glu Met Glu Lys Glu Gly Lys Ile Ser Arg Ile Gly Pro Glu Asn Pro Tyr Asn Thr Pro
Ile Phe Ala Ile Lys Lys Lys Asp Gly Thr Lys Trp Arg Lys Leu Val Asp Phe Arg Glu Leu Asn
Lys Lys Thr Gln Asp Phe Trp Glu Val Gln Leu Gly Ile Pro His Pro Ser Gly Leu Lys Lys Lys Lys
Ser Val Thr Val Leu Asp Ile Gly Asp Ala Tyr Phe Ser Val Pro Leu Asp Lys Glu Phe Arg Lys Tyr
Thr Ala Phe Thr Val Pro Ser Ala Ala Ala Trp Gly Leu Thr Thr Pro Asp Lys Lys His Gln Lys Asp
Pro Pro Phe Leu Trp Met Gly Tyr Glu Leu His Pro Asp Arg Trp Thr Val Gln Pro Ile Ala Ala Ala
Val Ala Lys Glu Ile Val Ala Cys Cys Asp Lys Cys Gln Leu Lys Gly Glu Ala Ile His Gly Gln Val
Asp Cys Ser Pro Gly Val Trp Gln Leu Asp Cys Thr His Leu Glu Gly Lys Val Ile Leu Val Ala Val
His Val Ala Ser Gly Tyr Met Glu Ala Glu Val Ile Pro Thr Glu Thr Gly Gln Glu Thr Ala Tyr Phe
Ile Leu Lys Leu Ala Gly Arg Trp Pro Val Thr Thr Ala Ala Ala Ala Val Lys Ala Ala Cys Trp Trp
Ala Gly Val Lys Gln Glu Phe Gly Ile Pro Tyr His Pro Gln Ser Gln Gly Val Val Glu Ser Met Asn
Asn Glu Leu Lys Lys Ile Ile Gly Gln Ile Arg Asp Gln Ala Glu Gln Leu Lys Thr Ala Val Gln Met
Ala Val Leu Ile His Asn Phe Lys Arg Lys Gly Gly Ile Gly Glu Tyr Ser Ala Gly Glu Arg Ile Ile
Asp Ile Ile Ala Ala Ala Ala Ile Thr Lys Leu Gln Asn Phe Arg Val Tyr Tyr Arg Asp Asn Arg Asp
Pro Leu Trp Lys Gly Pro Ala Arg Leu Leu Trp Lys Gly Glu Gly Ala Val Val Ile Gln Asp Asn Ser
Glu Ile Lys Val Val Pro Arg Arg Lys Val Lys Ile Ile Arg Asp Tyr Gly Lys Arg Met Ala Gly Asp
Asp Cys Val Ala Gly Arg Gln Asp Glu Asp Ala Ala Ala Glu Glu Val Gly Phe Pro Val Arg Pro
Gln Val Pro Leu Arg Pro Met Thr Tyr Lys Gly Ala Leu Asp Leu Ser His Phe Leu Lys Glu Lys
Gly Gly Leu Glu Gly
(SEQ ID NO: 347)
Leu Lys His Ile Val Trp Ala Ser Arg Glu Leu Glu Arg Phe Ala Val
Asn Pro Gly Leu Leu Glu Thr Val Ser Gln Asn Tyr Pro Ile Val Gln Asn Ile Ser Pro Arg Thr Leu
Asn Ala Trp Val Lys Val Val Glu Glu Lys Ala Phe Ser Pro Glu Val Ile Pro Met Phe Ser Ala Leu
Ser Glu Gly Ala Thr Pro Gln Asp Leu Asn Thr Met Leu Asn Thr Val Gly Gly His Gln Ala Ala
Met Gln Met Leu Lys Glu Thr Ile Asn Glu Glu Ala Ala Glu Trp Asp Arg Leu His Pro Val His
Ala Gly Pro Ile Ala Pro Gly Gln Met Arg Glu Pro Arg Gly Ser Asp Ile Ala Gly Thr Thr Ser Thr
Leu Gln Glu Gln Ile Gly Trp Met Thr Asn Asn Pro Pro Ile Pro Val Gly Glu Ile Tyr Lys Arg Trp
Ile Ile Leu Gly Leu Asn Lys Ile Val Arg Met Tyr Ser Pro Thr Ser Ile Leu Asp Ile Arg Gln Gly
Pro Lys Glu Pro Phe Arg Asp Tyr Val Asp Arg Phe Tyr Lys Thr Leu Arg Ala Glu Gln Ala Ser
Gln Glu Val Lys Asn Trp Met Thr Glu Thr Leu Leu Val Gln Asn Ala Asn Pro Asp Cys Lys Thr
Ile Leu Lys Ala Leu Gly Pro Ala Ala Thr Leu Glu Glu Met Met Thr Ala Cys Gln Gly Val Gly
Gly Pro Gly His Lys Ala Arg Val Leu Ala Glu Ala Met Ser Gln Arg Ala Lys Arg Ala Pro Val
Lys Gln Thr Leu Asn Phe Asp Leu Leu Lys Leu Ala Gly Asp Val Glu Ser Asn Pro Gly Pro Leu
Pro Gly Arg Trp Lys Pro Lys Met Ile Gly Gly Ile Gly Gly Phe Ile Lys Val Arg Gln Tyr Asp Gln
Gly Thr Val Leu Val Gly Pro Thr Pro Val Asn Ile Ile Gly Arg Asn Leu Leu Thr Gln Ile Gly Cys
Thr Leu Asn Phe Pro Ile Ser Pro Ile Glu Thr Val Pro Val Lys Leu Lys Pro Gly Met Asp Gly Pro
Lys Val Lys Gln Trp Pro Leu Thr Glu Glu Lys Ile Lys Ala Leu Val Glu Ile Cys Thr Glu Met Glu
Lys Glu Gly Lys Ile Ser Lys Ile Gly Pro Glu Asn Pro Tyr Asn Thr Pro Val Phe Ala Ile Lys Lys
Lys Asp Ser Thr Lys Trp Arg Lys Leu Val Asp Phe Arg Glu Leu Asn Lys Arg Thr Gln Asp Phe
Trp Glu Val Gln Leu Gly Ile Pro His Pro Ala Gly Leu Lys Lys Lys Lys Ser Val Thr Val Leu Asp
Val Gly Asp Ala Tyr Phe Ser Val Pro Leu Asp Lys Asp Phe Arg Lys Tyr Thr Ala Phe Thr Ile Pro
Ser Trp Gly Phe Thr Thr Pro Asp Lys Lys His Gln Lys Glu Pro Pro Phe Leu Trp Met Gly Tyr Glu
Leu His Pro Asp Lys Trp Thr Val Gln Pro Ile Val Ala Lys Glu Ile Val Ala Ser Cys Asp Lys Cys
Gln Leu Lys Gly Glu Ala Met His Gly Gln Val Asp Cys Ser Pro Gly Ile Trp Gln Leu Asp Cys
Thr His Leu Glu Gly Lys Ile Ile Leu Val Ala Val His Val Ala Ser Gly Tyr Ile Glu Ala Glu Val Ile
Pro Ala Glu Thr Gly Gln Glu Thr Ala Tyr Phe Leu Leu Lys Leu Ala Gly Arg Trp Pro Val Lys
Thr Thr Val Lys Ala Ala Cys Trp Trp Ala Gly Ile Lys Gln Glu Phe Gly Ile Pro Tyr Asn Pro Gln
Ser Gln Gly Val Val Glu Ser Met Asn Lys Glu Leu Lys Lys Ile Ile Gly Gln Val Arg Asp Gln Ala
Glu His Leu Lys Thr Ala Val Gln Met Ala Val Phe Ile His Asn Phe Lys Arg Lys Gly Gly Ile Gly
Gly Tyr Ser Ala Gly Glu Arg Ile Val Asp Ile Ile Ala Ile Thr Lys Ile Gln Asn Phe Arg Val Tyr Tyr
Arg Asp Ser Arg Asp Pro Leu Trp Lys Gly Pro Ala Lys Leu Leu Trp Lys Gly Glu Gly Ala Val
Val Ile Gln Asp Asn Ser Asp Ile Lys Val Val Pro Arg Arg Lys Ala Lys Ile Ile Arg Asp Tyr Gly
Lys Gln Met Ala Gly Asp Asp Cys Val Ala Ser Arg Gln Asp Glu Asp Arg Ala Lys Arg Ala Pro
Val Lys Gln Thr Leu Asn Phe Asp Leu Leu Lys Leu Ala Gly Asp Val Glu Ser Asn Pro Gly Pro
Glu Glu Val Gly Phe Pro Val Lys Pro Gln Val Pro Leu Arg Pro Met Thr Phe Lys Gly Ala Leu
Asp Leu Ser His Phe Leu Arg Glu Lys Gly Gly Leu Glu Gly
(SEQ ID NO: 348)
Leu Lys His Leu Val Trp Ala Ser Arg Glu Leu Glu Arg Phe Ala Leu
Asn Pro Gly Leu Leu Glu Thr Val Ser Gln Asn Phe Pro Ile Val Gln Asn Leu Ser Pro Arg Thr Leu
Asn Ala Trp Val Lys Val Ile Glu Glu Lys Ala Phe Ser Pro Glu Val Ile Pro Met Phe Thr Ala Leu
Ser Glu Gly Ala Thr Pro His Asp Leu Asn Thr Met Leu Asn Thr Ile Gly Gly His Gln Ala Ala Met
Gln Met Leu Lys Asp Thr Ile Asn Glu Glu Ala Ala Glu Trp Asp Arg Val His Pro Val His Ala Gly
Pro Val Ala Pro Gly Gln Met Arg Asp Pro Arg Gly Ser Asp Ile Ala Gly Ser Thr Ser Thr Leu Gln
Glu Gln Ile Ala Trp Met Thr Asn Asn Pro Pro Ile Pro Val Gly Asp Ile Tyr Lys Arg Trp Ile Ile
Met Gly Leu Asn Lys Ile Val Arg Met Tyr Ser Pro Val Ser Ile Leu Asp Ile Lys Gln Gly Pro Lys
Glu Pro Phe Arg Asp Tyr Val Asp Arg Phe Tyr Arg Thr Leu Arg Ala Glu Gln Ala Ser Gln Asp
Val Lys Asn Trp Met Thr Glu Thr Leu Leu Val Gln Asn Ser Asn Pro Asp Cys Lys Thr Ile Leu
Lys Ala Leu Gly Pro Gly Ala Thr Leu Glu Glu Met Met Ser Ala Cys Gln Gly Val Gly Gly Pro
Ser His Lys Ala Arg Val Leu Ala Glu Ala Met Cys Gln Arg Ala Lys Arg Ala Pro Val Lys Gln
Thr Leu Asn Phe Asp Leu Leu Lys Leu Ala Gly Asp Val Glu Ser Asn Pro Gly Pro Leu Pro Gly
Lys Trp Lys Pro Lys Met Ile Gly Gly Ile Gly Gly Phe Ile Lys Val Lys Gln Tyr Asp Gln Gly Thr
Val Leu Ile Gly Pro Thr Pro Val Asn Ile Ile Gly Arg Asn Leu Leu Thr Gln Leu Gly Cys Thr Leu
Asn Phe Pro Ile Ser Pro Ile Asp Thr Val Pro Val Lys Leu Lys Pro Gly Met Asp Gly Pro Arg Val
Lys Gln Trp Pro Leu Thr Glu Glu Lys Ile Lys Ala Leu Ile Glu Ile Cys Thr Glu Met Glu Lys Glu
Gly Lys Ile Ser Arg Ile Gly Pro Glu Asn Pro Tyr Asn Thr Pro Ile Phe Ala Ile Lys Lys Lys Asp
Gly Thr Lys Trp Arg Lys Leu Val Asp Phe Arg Glu Leu Asn Lys Lys Thr Gln Asp Phe Trp Glu
Val Gln Leu Gly Ile Pro His Pro Ser Gly Leu Lys Lys Lys Lys Ser Val Thr Val Leu Asp Ile Gly
Asp Ala Tyr Phe Ser Val Pro Leu Asp Lys Glu Phe Arg Lys Tyr Thr Ala Phe Thr Val Pro Ser Trp
Gly Leu Thr Thr Pro Asp Lys Lys His Gln Lys Asp Pro Pro Phe Leu Trp Met Gly Tyr Glu Leu
His Pro Asp Arg Trp Thr Val Gln Pro Ile Val Ala Lys Glu Ile Val Ala Cys Cys Asp Lys Cys Gln
Leu Lys Gly Glu Ala Ile His Gly Gln Val Asp Cys Ser Pro Gly Val Trp Gln Leu Asp Cys Thr His
Leu Glu Gly Lys Val Ile Leu Val Ala Val His Val Ala Ser Gly Tyr Met Glu Ala Glu Val Ile Pro
Thr Glu Thr Gly Gln Glu Thr Ala Tyr Phe Ile Leu Lys Leu Ala Gly Arg Trp Pro Val Thr Thr Ala
Val Lys Ala Ala Cys Trp Trp Ala Gly Val Lys Gln Glu Phe Gly Ile Pro Tyr His Pro Gln Ser Gln
Gly Val Val Glu Ser Met Asn Asn Glu Leu Lys Lys Ile Ile Gly Gln Ile Arg Asp Gln Ala Glu Gln
Leu Lys Thr Ala Val Gln Met Ala Val Leu Ile His Asn Phe Lys Arg Lys Gly Gly Ile Gly Glu Tyr
Ser Ala Gly Glu Arg Ile Ile Asp Ile Ile Ala Ile Thr Lys Leu Gln Asn Phe Arg Val Tyr Tyr Arg
Asp Asn Arg Asp Pro Leu Trp Lys Gly Pro Ala Arg Leu Leu Trp Lys Gly Glu Gly Ala Val Val
Ile Gln Asp Asn Ser Glu Ile Lys Val Val Pro Arg Arg Lys Val Lys Ile Ile Arg Asp Tyr Gly Lys
Arg Met Ala Gly Asp Asp Cys Val Ala Gly Arg Gln Asp Glu Asp Arg Ala Lys Arg Ala Pro Val
Lys Gln Thr Leu Asn Phe Asp Leu Leu Lys Leu Ala Gly Asp Val Glu Ser Asn Pro Gly Pro Glu
Glu Val Gly Phe Pro Val Arg Pro Gln Val Pro Leu Arg Pro Met Thr Tyr Lys Gly Ala Leu Asp
Leu Ser His Phe Leu Lys Glu Lys Gly Gly Leu Glu Gly
(SEQ ID NO: 349)
Leu Lys His Ile Val Trp Ala Ser Arg Glu Leu Glu Arg Phe Ala Val
Asn Pro Gly Leu Leu Glu Thr Val Ser Gln Asn Tyr Pro Ile Val Gln Asn Ile Ser Pro Arg Thr Leu
Asn Ala Trp Val Lys Val Val Glu Glu Lys Ala Phe Ser Pro Glu Val Ile Pro Met Phe Ser Ala Leu
Ser Glu Gly Ala Thr Pro Gln Asp Leu Asn Thr Met Leu Asn Thr Val Gly Gly His Gln Ala Ala
Met Gln Met Leu Lys Glu Thr Ile Asn Glu Glu Ala Ala Glu Trp Asp Arg Leu His Pro Val His
Ala Gly Pro Ile Ala Pro Gly Gln Met Arg Glu Pro Arg Gly Ser Asp Ile Ala Gly Thr Thr Ser Thr
Leu Gln Glu Gln Ile Gly Trp Met Thr Asn Asn Pro Pro Ile Pro Val Gly Glu Ile Tyr Lys Arg Trp
Ile Ile Leu Gly Leu Asn Lys Ile Val Arg Met Tyr Ser Pro Thr Ser Ile Leu Asp Ile Arg Gln Gly
Pro Lys Glu Pro Phe Arg Asp Tyr Val Asp Arg Phe Tyr Lys Thr Leu Arg Ala Glu Gln Ala Ser
Gln Glu Val Lys Asn Trp Met Thr Glu Thr Leu Leu Val Gln Asn Ala Asn Pro Asp Cys Lys Thr
Ile Leu Lys Ala Leu Gly Pro Ala Ala Thr Leu Glu Glu Met Met Thr Ala Cys Gln Gly Val Gly
Gly Pro Gly His Lys Ala Arg Val Leu Ala Glu Ala Met Ser Gln Leu Pro Gly Arg Trp Lys Pro
Lys Met Ile Gly Gly Ile Gly Gly Phe Ile Lys Val Arg Gln Tyr Asp Gln Gly Thr Val Leu Val Gly
Pro Thr Pro Val Asn Ile Ile Gly Arg Asn Leu Leu Thr Gln Ile Gly Cys Thr Leu Asn Phe Pro Ile
Ser Pro Ile Glu Thr Val Pro Val Lys Leu Lys Pro Gly Met Asp Gly Pro Lys Val Lys Gln Trp Pro
Leu Thr Glu Glu Lys Ile Lys Ala Leu Val Glu Ile Cys Thr Glu Met Glu Lys Glu Gly Lys Ile Ser
Lys Ile Gly Pro Glu Asn Pro Tyr Asn Thr Pro Val Phe Ala Ile Lys Lys Lys Asp Ser Thr Lys Trp
Arg Lys Leu Val Asp Phe Arg Glu Leu Asn Lys Arg Thr Gln Asp Phe Trp Glu Val Gln Leu Gly
Ile Pro His Pro Ala Gly Leu Lys Lys Lys Lys Ser Val Thr Val Leu Asp Val Gly Asp Ala Tyr Phe
Ser Val Pro Leu Asp Lys Asp Phe Arg Lys Tyr Thr Ala Phe Thr Ile Pro Ser Trp Gly Phe Thr Thr
Pro Asp Lys Lys His Gln Lys Glu Pro Pro Phe Leu Trp Met Gly Tyr Glu Leu His Pro Asp Lys
Trp Thr Val Gln Pro Ile Val Ala Lys Glu Ile Val Ala Ser Cys Asp Lys Cys Gln Leu Lys Gly Glu
Ala Met His Gly Gln Val Asp Cys Ser Pro Gly Ile Trp Gln Leu Asp Cys Thr His Leu Glu Gly Lys
Ile Ile Leu Val Ala Val His Val Ala Ser Gly Tyr Ile Glu Ala Glu Val Ile Pro Ala Glu Thr Gly Gln
Glu Thr Ala Tyr Phe Leu Leu Lys Leu Ala Gly Arg Trp Pro Val Lys Thr Thr Val Lys Ala Ala
Cys Trp Trp Ala Gly Ile Lys Gln Glu Phe Gly Ile Pro Tyr Asn Pro Gln Ser Gln Gly Val Val Glu
Ser Met Asn Lys Glu Leu Lys Lys Ile Ile Gly Gln Val Arg Asp Gln Ala Glu His Leu Lys Thr Ala
Val Gln Met Ala Val Phe Ile His Asn Phe Lys Arg Lys Gly Gly Ile Gly Gly Tyr Ser Ala Gly Glu
Arg Ile Val Asp Ile Ile Ala Ile Thr Lys Ile Gln Asn Phe Arg Val Tyr Tyr Arg Asp Ser Arg Asp
Pro Leu Trp Lys Gly Pro Ala Lys Leu Leu Trp Lys Gly Glu Gly Ala Val Val Ile Gln Asp Asn Ser
Asp Ile Lys Val Val Pro Arg Arg Lys Ala Lys Ile Ile Arg Asp Tyr Gly Lys Gln Met Ala Gly Asp
Asp Cys Val Ala Ser Arg Gln Asp Glu Asp Glu Glu Val Gly Phe Pro Val Lys Pro Gln Val Pro
Leu Arg Pro Met Thr Phe Lys Gly Ala Leu Asp Leu Ser His Phe Leu Arg Glu Lys Gly Gly Leu
Glu Gly
(SEQ ID NO: 350)
Leu Lys His Leu Val Trp Ala Ser Arg Glu Leu Glu Arg Phe Ala Leu
Asn Pro Gly Leu Leu Glu Thr Val Ser Gln Asn Phe Pro Ile Val Gln Asn Leu Ser Pro Arg Thr Leu
Asn Ala Trp Val Lys Val Ile Glu Glu Lys Ala Phe Ser Pro Glu Val Ile Pro Met Phe Thr Ala Leu
Ser Glu Gly Ala Thr Pro His Asp Leu Asn Thr Met Leu Asn Thr Ile Gly Gly His Gln Ala Ala Met
Gln Met Leu Lys Asp Thr Ile Asn Glu Glu Ala Ala Glu Trp Asp Arg Val His Pro Val His Ala Gly
Pro Val Ala Pro Gly Gln Met Arg Asp Pro Arg Gly Ser Asp Ile Ala Gly Ser Thr Ser Thr Leu Gln
Glu Gln Ile Ala Trp Met Thr Asn Asn Pro Pro Ile Pro Val Gly Asp Ile Tyr Lys Arg Trp Ile Ile
Met Gly Leu Asn Lys Ile Val Arg Met Tyr Ser Pro Val Ser Ile Leu Asp Ile Lys Gln Gly Pro Lys
Glu Pro Phe Arg Asp Tyr Val Asp Arg Phe Tyr Arg Thr Leu Arg Ala Glu Gln Ala Ser Gln Asp
Val Lys Asn Trp Met Thr Glu Thr Leu Leu Val Gln Asn Ser Asn Pro Asp Cys Lys Thr Ile Leu
Lys Ala Leu Gly Pro Gly Ala Thr Leu Glu Glu Met Met Ser Ala Cys Gln Gly Val Gly Gly Pro
Ser His Lys Ala Arg Val Leu Ala Glu Ala Met Cys Gln Leu Pro Gly Lys Trp Lys Pro Lys Met Ile
Gly Gly Ile Gly Gly Phe Ile Lys Val Lys Gln Tyr Asp Gln Gly Thr Val Leu Ile Gly Pro Thr Pro
Val Asn Ile Ile Gly Arg Asn Leu Leu Thr Gln Leu Gly Cys Thr Leu Asn Phe Pro Ile Ser Pro Ile
Asp Thr Val Pro Val Lys Leu Lys Pro Gly Met Asp Gly Pro Arg Val Lys Gln Trp Pro Leu Thr
Glu Glu Lys Ile Lys Ala Leu Ile Glu Ile Cys Thr Glu Met Glu Lys Glu Gly Lys Ile Ser Arg Ile
Gly Pro Glu Asn Pro Tyr Asn Thr Pro Ile Phe Ala Ile Lys Lys Lys Asp Gly Thr Lys Trp Arg Lys
Leu Val Asp Phe Arg Glu Leu Asn Lys Lys Thr Gln Asp Phe Trp Glu Val Gln Leu Gly Ile Pro
His Pro Ser Gly Leu Lys Lys Lys Lys Ser Val Thr Val Leu Asp Ile Gly Asp Ala Tyr Phe Ser Val
Pro Leu Asp Lys Glu Phe Arg Lys Tyr Thr Ala Phe Thr Val Pro Ser Trp Gly Leu Thr Thr Pro Asp
Lys Lys His Gln Lys Asp Pro Pro Phe Leu Trp Met Gly Tyr Glu Leu His Pro Asp Arg Trp Thr
Val Gln Pro Ile Val Ala Lys Glu Ile Val Ala Cys Cys Asp Lys Cys Gln Leu Lys Gly Glu Ala Ile
His Gly Gln Val Asp Cys Ser Pro Gly Val Trp Gln Leu Asp Cys Thr His Leu Glu Gly Lys Val Ile
Leu Val Ala Val His Val Ala Ser Gly Tyr Met Glu Ala Glu Val Ile Pro Thr Glu Thr Gly Gln Glu
Thr Ala Tyr Phe Ile Leu Lys Leu Ala Gly Arg Trp Pro Val Thr Thr Ala Val Lys Ala Ala Cys Trp
Trp Ala Gly Val Lys Gln Glu Phe Gly Ile Pro Tyr His Pro Gln Ser Gln Gly Val Val Glu Ser Met
Asn Asn Glu Leu Lys Lys Ile Ile Gly Gln Ile Arg Asp Gln Ala Glu Gln Leu Lys Thr Ala Val Gln
Met Ala Val Leu Ile His Asn Phe Lys Arg Lys Gly Gly Ile Gly Glu Tyr Ser Ala Gly Glu Arg Ile
Ile Asp Ile Ile Ala Ile Thr Lys Leu Gln Asn Phe Arg Val Tyr Tyr Arg Asp Asn Arg Asp Pro Leu
Trp Lys Gly Pro Ala Arg Leu Leu Trp Lys Gly Glu Gly Ala Val Val Ile Gln Asp Asn Ser Glu Ile
Lys Val Val Pro Arg Arg Lys Val Lys Ile Ile Arg Asp Tyr Gly Lys Arg Met Ala Gly Asp Asp
Cys Val Ala Gly Arg Gln Asp Glu Asp Glu Glu Val Gly Phe Pro Val Arg Pro Gln Val Pro Leu
Arg Pro Met Thr Tyr Lys Gly Ala Leu Asp Leu Ser His Phe Leu Lys Glu Lys Gly Gly Leu Glu
Gly
(SEQ ID NO: 351)
Met Gly Ala Arg Ala Ser Val Leu Ser Gly Gly Glu Leu Asp Arg Trp
Glu Lys Ile Arg Leu Arg Pro Gly Gly Lys Lys Lys Tyr Arg Leu Lys His Ile Val Trp Ala Ser Arg
Glu Leu Glu Arg Phe Ala Val Asn Pro Gly Leu Leu Glu Thr Ala Ala Ile Ser Pro Arg Thr Leu Asn
Ala Trp Val Lys Val Val Glu Glu Lys Ala Phe Ser Pro Glu Val Ile Pro Met Phe Ser Ala Leu Ser
Glu Gly Ala Thr Pro Gln Asp Leu Asn Thr Met Leu Asn Thr Val Gly Gly His Gln Ala Ala Met
Gln Met Leu Lys Glu Thr Ile Asn Glu Glu Ala Ala Glu Trp Asp Arg Leu His Pro Val His Ala Gly
Pro Ile Ala Pro Gly Gln Met Arg Glu Pro Arg Gly Ser Asp Ile Ala Gly Thr Thr Ser Thr Leu Gln
Glu Gln Ile Gly Trp Met Thr Asn Asn Pro Pro Ile Pro Val Gly Glu Ile Tyr Lys Arg Trp Ile Ile Leu
Gly Leu Asn Lys Ile Val Arg Met Tyr Ser Pro Thr Ser Ile Leu Asp Ile Arg Gln Gly Pro Lys Glu
Pro Phe Arg Asp Tyr Val Asp Arg Phe Tyr Lys Thr Leu Arg Ala Glu Gln Ala Ser Gln Glu Val
Lys Asn Trp Met Thr Glu Thr Leu Leu Val Gln Asn Ala Asn Pro Asp Cys Lys Thr Ile Leu Lys
Ala Leu Gly Pro Ala Ala Thr Leu Glu Glu Met Met Thr Ala Cys Gln Gly Val Gly Gly Pro Gly
His Lys Ala Arg Val Leu Ala Glu Ala Met Ser Gln Glu Glu Val Gly Phe Pro Val Lys Pro Gln Val
Pro Leu Arg Pro Met Thr Phe Lys Gly Ala Leu Asp Leu Ser His Phe Leu Arg Glu Lys Gly Gly
Leu Glu Gly Thr Gln Gly Phe Phe Pro Asp Trp Gln Asn Tyr Thr Pro Glu Pro Gly Ile Arg Phe Pro
Leu Thr Phe Gly Trp Cys Phe Lys Leu Val Pro Leu
(SEQ ID NO: 352)
Leu Ser Pro Arg Thr Leu Asn Ala Trp Val Lys Val Ile Glu Glu Lys
Ala Phe Ser Pro Glu Val Ile Pro Met Phe Thr Ala Leu Ser Glu Gly Ala Thr Pro His Asp Leu Asn
Thr Met Leu Asn Thr Ile Gly Gly His Gln Ala Ala Met Gln Met Leu Lys Asp Thr Ile Asn Glu
Glu Ala Ala Glu Trp Asp Arg Val His Pro Val His Ala Gly Pro Val Ala Pro Gly Gln Met Arg Asp
Pro Arg Gly Ser Asp Ile Ala Gly Ser Thr Ser Thr Leu Gln Glu Gln Ile Ala Trp Met Thr Asn Asn
Pro Pro Ile Pro Val Gly Asp Ile Tyr Lys Arg Trp Ile Ile Met Gly Leu Asn Lys Ile Val Arg Met
Tyr Ser Pro Val Ser Ile Leu Asp Ile Lys Gln Gly Pro Lys Glu Pro Phe Arg Asp Tyr Val Asp Arg
Phe Tyr Arg Thr Leu Arg Ala Glu Gln Ala Ser Gln Asp Val Lys Asn Trp Met Thr Glu Thr Leu
Leu Val Gln Asn Ser Asn Pro Asp Cys Lys Thr Ile Leu Lys Ala Leu Gly Pro Gly Ala Thr Leu
Glu Glu Met Met Ser Ala Cys Gln Gly Val Gly Gly Pro Ser His Lys Ala Arg Val Leu Ala Glu
Ala Met Cys Gln Met Gly Ala Arg Ala Ser Ile Leu Ser Gly Gly Lys Leu Asp Lys Trp Glu Lys Ile
Arg Leu Arg Pro Gly Gly Arg Lys Lys Tyr Lys Leu Lys His Ile Val Trp Ala Ser Arg Glu Leu Glu
Arg Phe Ala Val Asn Pro Gly Leu Leu Glu Thr Glu Glu Val Gly Phe Pro Val Arg Pro Gln Val
Pro Leu Arg Pro Met Thr Tyr Lys Gly Ala Leu Asp Leu Ser His Phe Leu Lys Glu Lys Gly Gly
Leu Glu Gly Thr Gln Gly Tyr Phe Pro Asp Trp Gln Asn Tyr Thr Pro Gly Pro Gly Thr Arg Tyr
Pro Leu Thr Phe Gly Trp Cys Phe Lys Leu Val Pro Val
(SEQ ID NO: 353)
Met Trp Leu Gln Ser Leu Leu Leu Leu Gly Thr Val Ala Cys Ser Ile
Ser Val Met Gly Ala Arg Ala Ser Val Leu Ser Gly Gly Glu Leu Asp Arg Trp Glu Lys Ile Arg Leu
Arg Pro Gly Gly Lys Lys Lys Tyr Arg Leu Lys His Ile Val Trp Ala Ser Arg Glu Leu Glu Arg Phe
Ala Val Asn Pro Gly Leu Leu Glu Thr Leu Lys His Ile Val Trp Ala Ser Arg Glu Leu Glu Arg Phe
Ala Val Asn Pro Gly Leu Leu Glu Thr Ala Ala Ile Ser Pro Arg Thr Leu Asn Ala Trp Val Lys Val
Val Glu Glu Lys Ala Phe Ser Pro Glu Val Ile Pro Met Phe Ser Ala Leu Ser Glu Gly Ala Thr Pro
Gln Asp Leu Asn Thr Met Leu Asn Thr Val Gly Gly His Gln Ala Ala Met Gln Met Leu Lys Glu
Thr Ile Asn Glu Glu Ala Ala Glu Trp Asp Arg Leu His Pro Val His Ala Gly Pro Ile Ala Pro Gly
Gln Met Arg Glu Pro Arg Gly Ser Asp Ile Ala Gly Thr Thr Ser Thr Leu Gln Glu Gln Ile Gly Trp
Met Thr Asn Asn Pro Pro Ile Pro Val Gly Glu Ile Tyr Lys Arg Trp Ile Ile Leu Gly Leu Asn Lys
Ile Val Arg Met Tyr Ser Pro Thr Ser Ile Leu Asp Ile Arg Gln Gly Pro Lys Glu Pro Phe Arg Asp
Tyr Val Asp Arg Phe Tyr Lys Thr Leu Arg Ala Glu Gln Ala Ser Gln Glu Val Lys Asn Trp Met
Thr Glu Thr Leu Leu Val Gln Asn Ala Asn Pro Asp Cys Lys Thr Ile Leu Lys Ala Leu Gly Pro
Ala Ala Thr Leu Glu Glu Met Met Thr Ala Cys Gln Gly Val Gly Gly Pro Gly His Lys Ala Arg
Val Leu Ala Glu Ala Met Ser Gln Glu Glu Val Gly Phe Pro Val Lys Pro Gln Val Pro Leu Arg Pro
Met Thr Phe Lys Gly Ala Leu Asp Leu Ser His Phe Leu Arg Glu Lys Gly Gly Leu Glu Gly Thr
Gln Gly Phe Phe Pro Asp Gln Asn Tyr Thr Pro Glu Pro Gly Ile Arg Phe Pro Leu Thr Phe Gly Trp
Cys Phe Lys Leu Val Pro Leu
(SEQ ID NO: 354)
Met Asp Ala Met Lys Arg Gly Leu Cys Cys Val Leu Leu Leu Cys
Gly Ala Val Phe Val Ser Ala Arg Met Gly Ala Arg Ala Ser Val Leu Ser Gly Gly Glu Leu Asp
Arg Trp Glu Lys Ile Arg Leu Arg Pro Gly Gly Lys Lys Lys Tyr Arg Leu Lys His Ile Val Trp Ala
Ser Arg Glu Leu Glu Arg Phe Ala Val Asn Pro Gly Leu Leu Glu Thr Leu Lys His Ile Val Trp Ala
Ser Arg Glu Leu Glu Arg Phe Ala Val Asn Pro Gly Leu Leu Glu Thr Ala Ala Ile Ser Pro Arg Thr
Leu Asn Ala Trp Val Lys Val Val Glu Glu Lys Ala Phe Ser Pro Glu Val Ile Pro Met Phe Ser Ala
Leu Ser Glu Gly Ala Thr Pro Gln Asp Leu Asn Thr Met Leu Asn Thr Val Gly Gly His Gln Ala
Ala Met Gln Met Leu Lys Glu Thr Ile Asn Glu Glu Ala Ala Glu Trp Asp Arg Leu His Pro Val
His Ala Gly Pro Ile Ala Pro Gly Gln Met Arg Glu Pro Arg Gly Ser Asp Ile Ala Gly Thr Thr Ser
Thr Leu Gln Glu Gln Ile Gly Trp Met Thr Asn Asn Pro Pro Ile Pro Val Gly Glu Ile Tyr Lys Arg
Trp Ile Ile Leu Gly Leu Asn Lys Ile Val Arg Met Tyr Ser Pro Thr Ser Ile Leu Asp Ile Arg Gln
Gly Pro Lys Glu Pro Phe Arg Asp Tyr Val Asp Arg Phe Tyr Lys Thr Leu Arg Ala Glu Gln Ala
Ser Gln Glu Val Lys Asn Trp Met Thr Glu Thr Leu Leu Val Gln Asn Ala Asn Pro Asp Cys Lys
Thr Ile Leu Lys Ala Leu Gly Pro Ala Ala Thr Leu Glu Glu Met Met Thr Ala Cys Gln Gly Val
Gly Gly Pro Gly His Lys Ala Arg Val Leu Ala Glu Ala Met Ser Gln Glu Glu Val Gly Phe Pro Val
Lys Pro Gln Val Pro Leu Arg Pro Met Thr Phe Lys Gly Ala Leu Asp Leu Ser His Phe Leu Arg
Glu Lys Gly Gly Leu Glu Gly Thr Gln Gly Phe Phe Pro Asp Gln Asn Tyr Thr Pro Glu Pro Gly
Ile Arg Phe Pro Leu Thr Phe Gly Trp Cys Phe Lys Leu Val Pro Leu
(SEQ ID NO: 355)
Met Asn Pro Ser Ala Ala Val Ile Phe Cys Leu Ile Leu Leu Gly Leu
Ser Gly Thr Gln Gly Ile Leu Asp Met Ala Gln Pro Val Gly Ile Asn Thr Ser Thr Thr Cys Cys Tyr
Arg Phe Ile Asn Lys Lys Ile Pro Lys Gln Arg Leu Glu Ser Tyr Arg Arg Thr Thr Ser Ser His Cys
Pro Arg Glu Ala Val Ile Phe Lys Thr Lys Leu Asp Lys Glu Ile Cys Ala Asp Pro Thr Gln Lys Trp
Val Gln Asp Phe Met Lys His Leu Asp Lys Lys Thr Gln Thr Pro Lys Leu Ala Ser Ala Gly Ala
Met Gly Ala Arg Ala Ser Val Leu Ser Gly Gly Glu Leu Asp Arg Trp Glu Lys Ile Arg Leu Arg
Pro Gly Gly Lys Lys Lys Tyr Arg Leu Lys His Ile Val Trp Ala Ser Arg Glu Leu Glu Arg Phe Ala
Val Asn Pro Gly Leu Leu Glu Thr Leu Lys His Ile Val Trp Ala Ser Arg Glu Leu Glu Arg Phe Ala
Val Asn Pro Gly Leu Leu Glu Thr Ala Ala Ile Ser Pro Arg Thr Leu Asn Ala Trp Val Lys Val Val
Glu Glu Lys Ala Phe Ser Pro Glu Val Ile Pro Met Phe Ser Ala Leu Ser Glu Gly Ala Thr Pro Gln
Asp Leu Asn Thr Met Leu Asn Thr Val Gly Gly His Gln Ala Ala Met Gln Met Leu Lys Glu Thr
Ile Asn Glu Glu Ala Ala Glu Trp Asp Arg Leu His Pro Val His Ala Gly Pro Ile Ala Pro Gly Gln
Met Arg Glu Pro Arg Gly Ser Asp Ile Ala Gly Thr Thr Ser Thr Leu Gln Glu Gln Ile Gly Trp Met
Thr Asn Asn Pro Pro Ile Pro Val Gly Glu Ile Tyr Lys Arg Trp Ile Ile Leu Gly Leu Asn Lys Ile
Val Arg Met Tyr Ser Pro Thr Ser Ile Leu Asp Ile Arg Gln Gly Pro Lys Glu Pro Phe Arg Asp Tyr
Val Asp Arg Phe Tyr Lys Thr Leu Arg Ala Glu Gln Ala Ser Gln Glu Val Lys Asn Trp Met Thr
Glu Thr Leu Leu Val Gln Asn Ala Asn Pro Asp Cys Lys Thr Ile Leu Lys Ala Leu Gly Pro Ala
Ala Thr Leu Glu Glu Met Met Thr Ala Cys Gln Gly Val Gly Gly Pro Gly His Lys Ala Arg Val
Leu Ala Glu Ala Met Ser Gln Glu Glu Val Gly Phe Pro Val Lys Pro Gln Val Pro Leu Arg Pro
Met Thr Phe Lys Gly Ala Leu Asp Leu Ser His Phe Leu Arg Glu Lys Gly Gly Leu Glu Gly Thr
Gln Gly Phe Phe Pro Asp Gln Asn Tyr Thr Pro Glu Pro Gly Ile Arg Phe Pro Leu Thr Phe Gly Trp
Cys Phe Lys Leu Val Pro Leu
(SEQ ID NO: 356)
Met Arg Lys Ala Ala Val Ser His Trp Gln Gln Gln Ser Tyr Leu Asp
Ser Gly Ile His Ser Gly Ala Thr Thr Thr Ala Pro Ser Leu Ser Met Gly Ala Arg Ala Ser Val Leu
Ser Gly Gly Glu Leu Asp Arg Trp Glu Lys Ile Arg Leu Arg Pro Gly Gly Lys Lys Lys Tyr Arg
Leu Lys His Ile Val Trp Ala Ser Arg Glu Leu Glu Arg Phe Ala Val Asn Pro Gly Leu Leu Glu Thr
Leu Lys His Ile Val Trp Ala Ser Arg Glu Leu Glu Arg Phe Ala Val Asn Pro Gly Leu Leu Glu Thr
Ala Ala Ile Ser Pro Arg Thr Leu Asn Ala Trp Val Lys Val Val Glu Glu Lys Ala Phe Ser Pro Glu
Val Ile Pro Met Phe Ser Ala Leu Ser Glu Gly Ala Thr Pro Gln Asp Leu Asn Thr Met Leu Asn Thr
Val Gly Gly His Gln Ala Ala Met Gln Met Leu Lys Glu Thr Ile Asn Glu Glu Ala Ala Glu Trp
Asp Arg Leu His Pro Val His Ala Gly Pro Ile Ala Pro Gly Gln Met Arg Glu Pro Arg Gly Ser Asp
Ile Ala Gly Thr Thr Ser Thr Leu Gln Glu Gln Ile Gly Trp Met Thr Asn Asn Pro Pro Ile Pro Val
Gly Glu Ile Tyr Lys Arg Trp Ile Ile Leu Gly Leu Asn Lys Ile Val Arg Met Tyr Ser Pro Thr Ser Ile
Leu Asp Ile Arg Gln Gly Pro Lys Glu Pro Phe Arg Asp Tyr Val Asp Arg Phe Tyr Lys Thr Leu
Arg Ala Glu Gln Ala Ser Gln Glu Val Lys Asn Trp Met Thr Glu Thr Leu Leu Val Gln Asn Ala
Asn Pro Asp Cys Lys Thr Ile Leu Lys Ala Leu Gly Pro Ala Ala Thr Leu Glu Glu Met Met Thr
Ala Cys Gln Gly Val Gly Gly Pro Gly His Lys Ala Arg Val Leu Ala Glu Ala Met Ser Gln Glu
Glu Val Gly Phe Pro Val Lys Pro Gln Val Pro Leu Arg Pro Met Thr Phe Lys Gly Ala Leu Asp
Leu Ser His Phe Leu Arg Glu Lys Gly Gly Leu Glu Gly Thr Gln Gly Phe Phe Pro Asp Gln Asn
Tyr Thr Pro Glu Pro Gly Ile Arg Phe Pro Leu Thr Phe Gly Trp Cys Phe Lys Leu Val Pro Leu
(SEQ ID NO: 357)
Gly Thr Val Leu Val Gly Pro Thr Pro Val Asn Ile Ile Gly Arg Asn
Leu Leu Thr Gln Ile Gly Cys Thr Leu Asn Phe Pro Ile Ser Pro Ile Glu Thr Val Pro Val Lys Leu
Lys Pro Gly Met Asp Gly Pro Lys Val Lys Gln Trp Pro Leu Thr Glu Glu Lys Ile Lys Ala Leu Val
Glu Ile Cys Thr Glu Met Glu Lys Glu Gly Lys Ile Ser Lys Ile Gly Pro Glu Asn Pro Tyr Asn Thr
Pro Val Phe Ala Ile Lys Lys Lys Asp Ser Thr Lys Trp Arg Lys Leu Val Asp Phe Arg Glu Leu
Asn Lys Arg Thr Gln Asp Phe Trp Glu Val Gln Leu Gly Ile Pro His Pro Ala Gly Leu Lys Lys Lys
Lys Ser Val Thr Val Leu Asp Val Gly Asp Ala Tyr Phe Ser Val Pro Leu Asp Lys Asp Phe Arg
Lys Tyr Thr Ala Phe Thr Ile Pro Ser Ile Asn Asn Glu Thr Pro Gly Ile Arg Tyr Gln Tyr Asn Val
Leu Pro Gln Gly Trp Lys Gly Ser Pro Ala Ile Phe Gln Ser Ser Met Thr Thr Val Lys Ala Ala Cys
Trp Trp Ala Gly Ile Lys Gln Glu Phe Gly Ile Pro Tyr Asn Pro Gln Ser Gln Gly Val Val Glu Ser
Met Asn Lys Glu Leu Lys Lys Ile Ile Gly Gln Val Arg Asp Gln Ala Glu His Leu Lys Thr Ala Val
Gln Met Ala Val Phe Ile His Asn Phe Lys Arg Lys Gly Gly Ile Gly Gly Tyr Ser Ala Gly Glu Arg
Ile Val Asp Ile Ile Asn Val Ser Thr Val Gln Cys Thr His Gly Ile Arg Pro Val Val Ser Thr Gln Leu
Leu Leu Asn Gly Ser Leu Ala Glu Glu Lys Arg Arg Val Val Gln Arg Glu Lys Arg Ala Val Gly
Ile Gly Ala Met Phe Leu Gly Phe Leu Gly Ala Ala Gly Ser Thr Met Gly Ala Ala Ser Ile Thr Leu
Thr Val Gln Ala Arg Gln Leu Leu Ser Gly Ile Val Gln Gln Gln Asn Asn Leu Leu Arg Ala Ile Glu
Ala Gln Gln His Leu Leu Gln Leu Thr Val Trp Gly Ile Lys Gln Leu Gln Ala Arg Val Leu Ala Val
Glu Arg Tyr Leu Lys Asp Gln Gln Leu Leu Gly Ile Trp Gly Cys Ser Gly Lys Leu Ile Cys Thr Thr
Val Ala Lys Glu Ile Val Ala Ser Cys Asp Lys Cys Gln Leu Lys Gly Glu Ala Met His Gly Gln Val
Asp Cys Ser Pro Gly Ile Trp Gln Leu Asp Cys Thr His Leu Glu Gly Lys Ile Ile Leu Val Ala Val
His Val Ala Ser Gly Tyr Ile Glu Ala Glu Val Ile Pro Ala Glu Thr Gly Gln Glu Thr Ala Tyr Phe
Leu Leu Lys Leu Ala Gly Arg Trp Pro Val Lys Thr Leu Trp Val Thr Val Tyr Tyr Gly Val Pro Val
Trp Lys Glu Ala Ala Phe Pro Gln Ile Thr Leu Trp Gln Arg Pro Leu Val Thr Ile Lys Ile Gly Gly
Gln Leu Lys Glu Ala Leu Leu Asp Thr Gly Ala Asp Asp Thr Val Leu Glu Glu Met Asn Leu Pro
Gly Arg Trp Lys Pro Lys Met Ile Gly Gly Ile Gly Gly Phe Ile Lys Val Arg Gln Tyr Asp Gln Ala
Ala Ala Ala His Asn Val Trp Ala Thr His Ala Cys Val Pro Thr Asp Pro Asn Pro Gln Glu Ala Ile
Thr Lys Ile Gln Asn Phe Arg Val Tyr Tyr Arg Asp Ser Arg Asp Pro Leu Trp Lys Gly Pro Ala Lys
Leu Leu Trp Lys Gly Glu Gly Ala Val Val Ile Gln Asp Asn Ser Asp Ile Lys Val Val Pro Arg Arg
Lys Ala Lys Ile Ile Arg Asp Tyr Gly Lys Gln Met Ala Gly Asp Asp Cys Val Ala Ser Arg Gln Asp
Glu Asp Pro Lys Phe Lys Leu Pro Ile Gln Lys Glu Thr Trp Glu Thr Trp Trp Thr Glu Tyr Trp Gln
Ala Thr Trp Ile Pro Glu Trp Glu Phe Val Asn Thr Pro Pro Leu Val Lys Leu Trp Tyr Gln Leu Glu
Lys Glu Pro Ile Val Gly Ala Glu Thr Phe Tyr Val Asp Gly Ala Ala Asn Arg Glu Thr Lys Ala Ala
Lys Glu Lys Val Tyr Leu Ala Trp Val Pro Ala His Lys Gly Ile Gly Gly Asn Glu Gln Val Asp Lys
Leu Val Ser Trp Gly Phe Thr Thr Pro Asp Lys Lys His Gln Lys Glu Pro Pro Phe Leu Trp Met
Gly Tyr Glu Leu His Pro Asp Lys Trp Thr Val Gln Pro Ile Val Leu Pro Glu Lys Asp Ser Trp Thr
Val Asn Asp Ile Gln Lys Leu Val Gly Lys Leu Asn Trp Ala Ser Gln Ile Tyr Pro Gly Ile Lys Val
Ile Val Ile Tyr Gln Tyr Met Asp Asp Leu Tyr Val Gly Ser Asp Leu Glu Ile Gly Gln His Arg Met
Arg Asp Asn Trp Arg Ser Glu Leu Tyr Lys Tyr Lys Val Val
(SEQ ID NO: 358)
Ala Val Lys Ala Ala Cys Trp Trp Ala Gly Val Lys Gln Glu Phe Gly
Ile Pro Tyr His Pro Gln Ser Gln Gly Val Val Glu Ser Met Asn Asn Glu Leu Lys Lys Ile Ile Gly
Gln Ile Arg Asp Gln Ala Glu Gln Leu Lys Thr Ala Val Gln Met Ala Val Leu Ile His Asn Phe Lys
Arg Lys Gly Gly Ile Gly Glu Tyr Ser Ala Gly Glu Arg Ile Ile Asp Ile Ile Arg Arg Arg Val Val
Gln Arg Glu Lys Arg Ala Ile Gly Leu Gly Ala Val Phe Leu Gly Phe Leu Gly Thr Ala Gly Ser Thr
Met Gly Ala Ala Ser Met Thr Leu Thr Val Gln Ala Arg Leu Leu Leu Ser Gly Ile Val Gln Gln Gln
Ser Asn Leu Leu Arg Ala Ile Glu Ala Gln Gln His Met Leu Gln Leu Thr Val Trp Gly Ile Lys Gln
Leu Gln Ala Arg Ile Leu Ala Val Glu Arg Tyr Leu Arg Asp Gln Gln Leu Leu Gly Ile Trp Gly
Cys Ser Gly Arg Leu Ile Cys Thr Thr Val Ala Lys Glu Ile Val Ala Cys Cys Asp Lys Cys Gln Leu
Lys Gly Glu Ala Ile His Gly Gln Val Asp Cys Ser Pro Gly Val Trp Gln Leu Asp Cys Thr His Leu
Glu Gly Lys Val Ile Leu Val Ala Val His Val Ala Ser Gly Tyr Met Glu Ala Glu Val Ile Pro Thr
Glu Thr Gly Gln Glu Thr Ala Tyr Phe Ile Leu Lys Leu Ala Gly Arg Trp Pro Val Thr Thr Asn Ile
Ser Thr Val Gln Cys Thr His Gly Ile Lys Pro Val Val Ser Thr Gln Leu Leu Leu Asn Gly Ser Leu
Ala Glu Lys Trp Gly Leu Thr Thr Pro Asp Lys Lys His Gln Lys Asp Pro Pro Phe Leu Trp Met
Gly Tyr Glu Leu His Pro Asp Arg Trp Thr Val Gln Pro Ile Glu Leu Pro Glu Lys Glu Ser Trp Thr
Val Asn Asp Ile Gln Lys Leu Ile Gly Lys Leu Asn Trp Ala Ser Gln Ile Tyr Ala Gly Ile Lys Val
Ile Val Ile Tyr Gln Tyr Val Asp Asp Leu Tyr Val Gly Ser Asp Leu Glu Ile Glu Gln His Arg Pro
Lys Phe Arg Leu Pro Ile Gln Lys Glu Thr Trp Asp Thr Trp Trp Thr Asp Tyr Trp Gln Ala Thr Trp
Ile Pro Glu Trp Glu Phe Thr Asn Thr Pro Pro Leu Val Lys Leu Trp Tyr Gln Leu Glu Thr Glu Pro
Ile Ala Gly Val Glu Thr Phe Tyr Val Asp Gly Ala Ser Asn Arg Glu Thr Lys Leu Pro Gln Ile Thr
Leu Trp Gln Arg Pro Ile Val Thr Ile Lys Ile Gly Gly Gln Ile Lys Glu Ala Leu Leu Asp Thr Gly
Ala Asp Asp Thr Val Leu Glu Asp Met Asn Leu Pro Gly Lys Trp Lys Pro Lys Met Ile Gly Gly
Ile Gly Gly Phe Ile Lys Val Lys Gln Tyr Asp Gln Ala Ala Leu Trp Val Thr Ile Tyr Tyr Gly Val
Pro Val Trp Lys Asp Val His Asn Ile Trp Ala Thr His Ala Cys Val Pro Thr Asp Pro Ser Pro Gln
Glu Ala Ile Thr Lys Leu Gln Asn Phe Arg Val Tyr Tyr Arg Asp Asn Arg Asp Pro Leu Trp Lys
Gly Pro Ala Arg Leu Leu Trp Lys Gly Glu Gly Ala Val Val Ile Gln Asp Asn Ser Glu Ile Lys Val
Val Pro Arg Arg Lys Val Lys Ile Ile Arg Asp Tyr Gly Lys Arg Met Ala Gly Asp Asp Cys Val Ala
Gly Arg Gln Asp Glu Asp Gly Thr Val Leu Ile Gly Pro Thr Pro Val Asn Ile Ile Gly Arg Asn Leu
Leu Thr Gln Leu Gly Cys Thr Leu Asn Phe Pro Ile Ser Pro Ile Asp Thr Val Pro Val Lys Leu Lys
Pro Gly Met Asp Gly Pro Arg Val Lys Gln Trp Pro Leu Thr Glu Glu Lys Ile Lys Ala Leu Ile Glu
Ile Cys Thr Glu Met Glu Lys Glu Gly Lys Ile Ser Arg Ile Gly Pro Glu Asn Pro Tyr Asn Thr Pro
Ile Phe Ala Ile Lys Lys Lys Asp Gly Thr Lys Trp Arg Lys Leu Val Asp Phe Arg Glu Leu Asn
Lys Lys Thr Gln Asp Phe Trp Glu Val Gln Leu Gly Ile Pro His Pro Ser Gly Leu Lys Lys Lys Lys
Ser Val Thr Val Leu Asp Ile Gly Asp Ala Tyr Phe Ser Val Pro Leu Asp Lys Glu Phe Arg Lys Tyr
Thr Ala Phe Thr Val Pro Ser Thr Asn Asn Glu Thr Pro Gly Val Arg Tyr Gln Tyr Asn Val Leu
Pro Met Gly Trp Lys Gly Ser Pro Ala Ile Phe Gln Cys Ser Met Thr Lys Glu Lys Ile Tyr Leu Ala
Trp Val Pro Ala His Lys Gly Ile Gly Gly Asn Glu Gln Ile Asp Lys Leu Val Ser Met Lys Asp Asn
Trp Arg Ser Glu Leu Tyr Arg Tyr Lys Val Val
(SEQ ID NO: 359)
Gly Thr Val Leu Val Gly Pro Thr Pro Val Asn Ile Ile Gly Arg Asn
Leu Leu Thr Gln Ile Gly Cys Thr Leu Asn Phe Pro Ile Ser Pro Ile Glu Thr Val Pro Val Lys Leu
Lys Pro Gly Met Asp Gly Pro Lys Val Lys Gln Trp Pro Leu Thr Glu Glu Lys Ile Lys Ala Leu Val
Glu Ile Cys Thr Glu Met Glu Lys Glu Gly Lys Ile Ser Lys Ile Gly Pro Glu Asn Pro Tyr Asn Thr
Pro Val Phe Ala Ile Lys Lys Lys Asp Ser Thr Lys Trp Arg Lys Leu Val Asp Phe Arg Glu Leu
Asn Lys Arg Thr Gln Asp Phe Trp Glu Val Gln Leu Gly Ile Pro His Pro Ala Gly Leu Lys Lys Lys
Lys Ser Val Thr Val Leu Asp Val Gly Asp Ala Tyr Phe Ser Val Pro Leu Asp Lys Asp Phe Arg
Lys Tyr Thr Ala Phe Thr Ile Pro Ser Ile Asn Asn Glu Thr Pro Gly Ile Arg Tyr Gln Tyr Asn Val
Leu Pro Gln Gly Trp Lys Gly Ser Pro Ala Ile Phe Gln Ser Ser Met Thr Thr Val Lys Ala Ala Cys
Trp Trp Ala Gly Ile Lys Gln Glu Phe Gly Ile Pro Tyr Asn Pro Gln Ser Gln Gly Val Val Glu Ser
Met Asn Lys Glu Leu Lys Lys Ile Ile Gly Gln Val Arg Asp Gln Ala Glu His Leu Lys Thr Ala Val
Gln Met Ala Val Phe Ile His Asn Phe Lys Arg Lys Gly Gly Ile Gly Gly Tyr Ser Ala Gly Glu Arg
Ile Val Asp Ile Ile Asn Val Ser Thr Val Gln Cys Thr His Gly Ile Arg Pro Val Val Ser Thr Gln Leu
Leu Leu Asn Gly Ser Leu Ala Glu Glu Lys Arg Arg Val Val Gln Arg Glu Lys Arg Ala Val Gly
Ile Gly Ala Met Phe Leu Gly Phe Leu Gly Ala Ala Gly Ser Thr Met Gly Ala Ala Ser Ile Thr Leu
Thr Val Gln Ala Arg Gln Leu Leu Ser Gly Ile Val Gln Gln Gln Asn Asn Leu Leu Arg Ala Ile Glu
Ala Gln Gln His Leu Leu Gln Leu Thr Val Trp Gly Ile Lys Gln Leu Gln Ala Arg Val Leu Ala Val
Glu Arg Tyr Leu Lys Asp Gln Gln Leu Leu Gly Ile Trp Gly Cys Ser Gly Lys Leu Ile Cys Thr Thr
Val Ala Lys Glu Ile Val Ala Ser Cys Asp Lys Cys Gln Leu Lys Gly Glu Ala Met His Gly Gln Val
Asp Cys Ser Pro Gly Ile Trp Gln Leu Asp Cys Thr His Leu Glu Gly Lys Ile Ile Leu Val Ala Val
His Val Ala Ser Gly Tyr Ile Glu Ala Glu Val Ile Pro Ala Glu Thr Gly Gln Glu Thr Ala Tyr Phe
Leu Leu Lys Leu Ala Gly Arg Trp Pro Val Lys Thr
(SEQ ID NO: 360)
Leu Trp Val Thr Val Tyr Tyr Gly Val Pro Val Trp Lys Glu Ala Ala
Phe Pro Gln Ile Thr Leu Trp Gln Arg Pro Leu Val Thr Ile Lys Ile Gly Gly Gln Leu Lys Glu Ala
Leu Leu Asp Thr Gly Ala Asp Asp Thr Val Leu Glu Glu Met Asn Leu Pro Gly Arg Trp Lys Pro
Lys Met Ile Gly Gly Ile Gly Gly Phe Ile Lys Val Arg Gln Tyr Asp Gln Ala Ala Ala Ala His Asn
Val Trp Ala Thr His Ala Cys Val Pro Thr Asp Pro Asn Pro Gln Glu Ala Ile Thr Lys Ile Gln Asn
Phe Arg Val Tyr Tyr Arg Asp Ser Arg Asp Pro Leu Trp Lys Gly Pro Ala Lys Leu Leu Trp Lys
Gly Glu Gly Ala Val Val Ile Gln Asp Asn Ser Asp Ile Lys Val Val Pro Arg Arg Lys Ala Lys Ile
Ile Arg Asp Tyr Gly Lys Gln Met Ala Gly Asp Asp Cys Val Ala Ser Arg Gln Asp Glu Asp Pro
Lys Phe Lys Leu Pro Ile Gln Lys Glu Thr Trp Glu Thr Trp Trp Thr Glu Tyr Trp Gln Ala Thr Trp
Ile Pro Glu Trp Glu Phe Val Asn Thr Pro Pro Leu Val Lys Leu Trp Tyr Gln Leu Glu Lys Glu Pro
Ile Val Gly Ala Glu Thr Phe Tyr Val Asp Gly Ala Ala Asn Arg Glu Thr Lys Ala Ala Lys Glu Lys
Val Tyr Leu Ala Trp Val Pro Ala His Lys Gly Ile Gly Gly Asn Glu Gln Val Asp Lys Leu Val Ser
Trp Gly Phe Thr Thr Pro Asp Lys Lys His Gln Lys Glu Pro Pro Phe Leu Trp Met Gly Tyr Glu
Leu His Pro Asp Lys Trp Thr Val Gln Pro Ile Val Leu Pro Glu Lys Asp Ser Trp Thr Val Asn Asp
Ile Gln Lys Leu Val Gly Lys Leu Asn Trp Ala Ser Gln Ile Tyr Pro Gly Ile Lys Val Ile Val Ile Tyr
Gln Tyr Met Asp Asp Leu Tyr Val Gly Ser Asp Leu Glu Ile Gly Gln His Arg Met Arg Asp Asn
Trp Arg Ser Glu Leu Tyr Lys Tyr Lys Val Val
(SEQ ID NO: 361)
Ala Val Lys Ala Ala Cys Trp Trp Ala Gly Val Lys Gln Glu Phe Gly
Ile Pro Tyr His Pro Gln Ser Gln Gly Val Val Glu Ser Met Asn Asn Glu Leu Lys Lys Ile Ile Gly
Gln Ile Arg Asp Gln Ala Glu Gln Leu Lys Thr Ala Val Gln Met Ala Val Leu Ile His Asn Phe Lys
Arg Lys Gly Gly Ile Gly Glu Tyr Ser Ala Gly Glu Arg Ile Ile Asp Ile Ile Arg Arg Arg Val Val
Gln Arg Glu Lys Arg Ala Ile Gly Leu Gly Ala Val Phe Leu Gly Phe Leu Gly Thr Ala Gly Ser Thr
Met Gly Ala Ala Ser Met Thr Leu Thr Val Gln Ala Arg Leu Leu Leu Ser Gly Ile Val Gln Gln Gln
Ser Asn Leu Leu Arg Ala Ile Glu Ala Gln Gln His Met Leu Gln Leu Thr Val Trp Gly Ile Lys Gln
Leu Gln Ala Arg Ile Leu Ala Val Glu Arg Tyr Leu Arg Asp Gln Gln Leu Leu Gly Ile Trp Gly
Cys Ser Gly Arg Leu Ile Cys Thr Thr Val Ala Lys Glu Ile Val Ala Cys Cys Asp Lys Cys Gln Leu
Lys Gly Glu Ala Ile His Gly Gln Val Asp Cys Ser Pro Gly Val Trp Gln Leu Asp Cys Thr His Leu
Glu Gly Lys Val Ile Leu Val Ala Val His Val Ala Ser Gly Tyr Met Glu Ala Glu Val Ile Pro Thr
Glu Thr Gly Gln Glu Thr Ala Tyr Phe Ile Leu Lys Leu Ala Gly Arg Trp Pro Val Thr Thr Asn Ile
Ser Thr Val Gln Cys Thr His Gly Ile Lys Pro Val Val Ser Thr Gln Leu Leu Leu Asn Gly Ser Leu
Ala Glu Lys Trp Gly Leu Thr Thr Pro Asp Lys Lys His Gln Lys Asp Pro Pro Phe Leu Trp Met
Gly Tyr Glu Leu His Pro Asp Arg Trp Thr Val Gln Pro Ile Glu Leu Pro Glu Lys Glu Ser Trp Thr
Val Asn Asp Ile Gln Lys Leu Ile Gly Lys Leu Asn Trp Ala Ser Gln Ile Tyr Ala Gly Ile Lys Val
Ile Val Ile Tyr Gln Tyr Val Asp Asp Leu Tyr Val Gly Ser Asp Leu Glu Ile Glu Gln His Arg Pro
Lys Phe Arg Leu Pro Ile Gln Lys Glu Thr Trp Asp Thr Trp Trp Thr Asp Tyr Trp Gln Ala Thr Trp
Ile Pro Glu Trp Glu Phe Thr Asn Thr Pro Pro Leu Val Lys Leu Trp Tyr Gln Leu Glu Thr Glu Pro
Ile Ala Gly Val Glu Thr Phe Tyr Val Asp Gly Ala Ser Asn Arg Glu Thr Lys Leu Pro Gln Ile Thr
Leu Trp Gln Arg Pro Ile Val Thr Ile Lys Ile Gly Gly Gln Ile Lys Glu Ala Leu Leu Asp Thr Gly
Ala Asp Asp Thr Val Leu Glu Asp Met Asn Leu Pro Gly Lys Trp Lys Pro Lys Met Ile Gly Gly
Ile Gly Gly Phe Ile Lys Val Lys Gln Tyr Asp Gln Ala Ala
(SEQ ID NO: 362)
Leu Trp Val Thr Ile Tyr Tyr Gly Val Pro Val Trp Lys Asp Val His
Asn Ile Trp Ala Thr His Ala Cys Val Pro Thr Asp Pro Ser Pro Gln Glu Ala Ile Thr Lys Leu Gln
Asn Phe Arg Val Tyr Tyr Arg Asp Asn Arg Asp Pro Leu Trp Lys Gly Pro Ala Arg Leu Leu Trp
Lys Gly Glu Gly Ala Val Val Ile Gln Asp Asn Ser Glu Ile Lys Val Val Pro Arg Arg Lys Val Lys
Ile Ile Arg Asp Tyr Gly Lys Arg Met Ala Gly Asp Asp Cys Val Ala Gly Arg Gln Asp Glu Asp
Gly Thr Val Leu Ile Gly Pro Thr Pro Val Asn Ile Ile Gly Arg Asn Leu Leu Thr Gln Leu Gly Cys
Thr Leu Asn Phe Pro Ile Ser Pro Ile Asp Thr Val Pro Val Lys Leu Lys Pro Gly Met Asp Gly Pro
Arg Val Lys Gln Trp Pro Leu Thr Glu Glu Lys Ile Lys Ala Leu Ile Glu Ile Cys Thr Glu Met Glu
Lys Glu Gly Lys Ile Ser Arg Ile Gly Pro Glu Asn Pro Tyr Asn Thr Pro Ile Phe Ala Ile Lys Lys
Lys Asp Gly Thr Lys Trp Arg Lys Leu Val Asp Phe Arg Glu Leu Asn Lys Lys Thr Gln Asp Phe
Trp Glu Val Gln Leu Gly Ile Pro His Pro Ser Gly Leu Lys Lys Lys Lys Ser Val Thr Val Leu Asp
Ile Gly Asp Ala Tyr Phe Ser Val Pro Leu Asp Lys Glu Phe Arg Lys Tyr Thr Ala Phe Thr Val Pro
Ser Thr Asn Asn Glu Thr Pro Gly Val Arg Tyr Gln Tyr Asn Val Leu Pro Met Gly Trp Lys Gly
Ser Pro Ala Ile Phe Gln Cys Ser Met Thr Lys Glu Lys Ile Tyr Leu Ala Trp Val Pro Ala His Lys
Gly Ile Gly Gly Asn Glu Gln Ile Asp Lys Leu Val Ser Met Lys Asp Asn Trp Arg Ser Glu Leu Tyr
Arg Tyr Lys Val Val
(SEQ ID NO: 363)
Met Trp Leu Gln Ser Leu Leu Leu Leu Gly Thr Val Ala Cys Ser Ile
Ser Val Gly Thr Val Leu Val Gly Pro Thr Pro Val Asn Ile Ile Gly Arg Asn Leu Leu Thr Gln Ile
Gly Cys Thr Leu Asn Phe Pro Ile Ser Pro Ile Glu Thr Val Pro Val Lys Leu Lys Pro Gly Met Asp
Gly Pro Lys Val Lys Gln Trp Pro Leu Thr Glu Glu Lys Ile Lys Ala Leu Val Glu Ile Cys Thr Glu
Met Glu Lys Glu Gly Lys Ile Ser Lys Ile Gly Pro Glu Asn Pro Tyr Asn Thr Pro Val Phe Ala Ile
Lys Lys Lys Asp Ser Thr Lys Trp Arg Lys Leu Val Asp Phe Arg Glu Leu Asn Lys Arg Thr Gln
Asp Phe Trp Glu Val Gln Leu Gly Ile Pro His Pro Ala Gly Leu Lys Lys Lys Lys Ser Val Thr Val
Leu Asp Val Gly Asp Ala Tyr Phe Ser Val Pro Leu Asp Lys Asp Phe Arg Lys Tyr Thr Ala Phe
Thr Ile Pro Ser Ile Asn Asn Glu Thr Pro Gly Ile Arg Tyr Gln Tyr Asn Val Leu Pro Gln Gly Trp
Lys Gly Ser Pro Ala Ile Phe Gln Ser Ser Met Thr Thr Val Lys Ala Ala Cys Trp Trp Ala Gly Ile
Lys Gln Glu Phe Gly Ile Pro Tyr Asn Pro Gln Ser Gln Gly Val Val Glu Ser Met Asn Lys Glu Leu
Lys Lys Ile Ile Gly Gln Val Arg Asp Gln Ala Glu His Leu Lys Thr Ala Val Gln Met Ala Val Phe
Ile His Asn Phe Lys Arg Lys Gly Gly Ile Gly Gly Tyr Ser Ala Gly Glu Arg Ile Val Asp Ile Ile
Asn Val Ser Thr Val Gln Cys Thr His Gly Ile Arg Pro Val Val Ser Thr Gln Leu Leu Leu Asn Gly
Ser Leu Ala Glu Glu Lys Arg Arg Val Val Gln Arg Glu Lys Arg Ala Val Gly Ile Gly Ala Met
Phe Leu Gly Phe Leu Gly Ala Ala Gly Ser Thr Met Gly Ala Ala Ser Ile Thr Leu Thr Val Gln Ala
Arg Gln Leu Leu Ser Gly Ile Val Gln Gln Gln Asn Asn Leu Leu Arg Ala Ile Glu Ala Gln Gln His
Leu Leu Gln Leu Thr Val Trp Gly Ile Lys Gln Leu Gln Ala Arg Val Leu Ala Val Glu Arg Tyr
Leu Lys Asp Gln Gln Leu Leu Gly Ile Trp Gly Cys Ser Gly Lys Leu Ile Cys Thr Thr Val Ala Lys
Glu Ile Val Ala Ser Cys Asp Lys Cys Gln Leu Lys Gly Glu Ala Met His Gly Gln Val Asp Cys
Ser Pro Gly Ile Trp Gln Leu Asp Cys Thr His Leu Glu Gly Lys Ile Ile Leu Val Ala Val His Val
Ala Ser Gly Tyr Ile Glu Ala Glu Val Ile Pro Ala Glu Thr Gly Gln Glu Thr Ala Tyr Phe Leu Leu
Lys Leu Ala Gly Arg Trp Pro Val Lys Thr Leu Trp Val Thr Val Tyr Tyr Gly Val Pro Val Trp Lys
Glu Ala Ala Phe Pro Gln Ile Thr Leu Trp Gln Arg Pro Leu Val Thr Ile Lys Ile Gly Gly Gln Leu
Lys Glu Ala Leu Leu Asp Thr Gly Ala Asp Asp Thr Val Leu Glu Glu Met Asn Leu Pro Gly Arg
Trp Lys Pro Lys Met Ile Gly Gly Ile Gly Gly Phe Ile Lys Val Arg Gln Tyr Asp Gln Ala Ala Ala
Ala His Asn Val Trp Ala Thr His Ala Cys Val Pro Thr Asp Pro Asn Pro Gln Glu Ala Ile Thr Lys
Ile Gln Asn Phe Arg Val Tyr Tyr Arg Asp Ser Arg Asp Pro Leu Trp Lys Gly Pro Ala Lys Leu
Leu Trp Lys Gly Glu Gly Ala Val Val Ile Gln Asp Asn Ser Asp Ile Lys Val Val Pro Arg Arg Lys
Ala Lys Ile Ile Arg Asp Tyr Gly Lys Gln Met Ala Gly Asp Asp Cys Val Ala Ser Arg Gln Asp Glu
Asp Pro Lys Phe Lys Leu Pro Ile Gln Lys Glu Thr Trp Glu Thr Trp Trp Thr Glu Tyr Trp Gln Ala
Thr Trp Ile Pro Glu Trp Glu Phe Val Asn Thr Pro Pro Leu Val Lys Leu Trp Tyr Gln Leu Glu Lys
Glu Pro Ile Val Gly Ala Glu Thr Phe Tyr Val Asp Gly Ala Ala Asn Arg Glu Thr Lys Ala Ala Lys
Glu Lys Val Tyr Leu Ala Trp Val Pro Ala His Lys Gly Ile Gly Gly Asn Glu Gln Val Asp Lys Leu
Val Ser Trp Gly Phe Thr Thr Pro Asp Lys Lys His Gln Lys Glu Pro Pro Phe Leu Trp Met Gly Tyr
Glu Leu His Pro Asp Lys Trp Thr Val Gln Pro Ile Val Leu Pro Glu Lys Asp Ser Trp Thr Val Asn
Asp Ile Gln Lys Leu Val Gly Lys Leu Asn Trp Ala Ser Gln Ile Tyr Pro Gly Ile Lys Val Ile Val Ile
Tyr Gln Tyr Met Asp Asp Leu Tyr Val Gly Ser Asp Leu Glu Ile Gly Gln His Arg Met Arg Asp
Asn Trp Arg Ser Glu Leu Tyr Lys Tyr Lys Val Val
(SEQ ID NO: 364)
Met Asp Ala Met Lys Arg Gly Leu Cys Cys Val Leu Leu Leu Cys
Gly Ala Val Phe Val Ser Ala Arg Gly Thr Val Leu Val Gly Pro Thr Pro Val Asn Ile Ile Gly Arg
Asn Leu Leu Thr Gln Ile Gly Cys Thr Leu Asn Phe Pro Ile Ser Pro Ile Glu Thr Val Pro Val Lys
Leu Lys Pro Gly Met Asp Gly Pro Lys Val Lys Gln Trp Pro Leu Thr Glu Glu Lys Ile Lys Ala Leu
Val Glu Ile Cys Thr Glu Met Glu Lys Glu Gly Lys Ile Ser Lys Ile Gly Pro Glu Asn Pro Tyr Asn
Thr Pro Val Phe Ala Ile Lys Lys Lys Asp Ser Thr Lys Trp Arg Lys Leu Val Asp Phe Arg Glu Leu
Asn Lys Arg Thr Gln Asp Phe Trp Glu Val Gln Leu Gly Ile Pro His Pro Ala Gly Leu Lys Lys Lys
Lys Ser Val Thr Val Leu Asp Val Gly Asp Ala Tyr Phe Ser Val Pro Leu Asp Lys Asp Phe Arg
Lys Tyr Thr Ala Phe Thr Ile Pro Ser Ile Asn Asn Glu Thr Pro Gly Ile Arg Tyr Gln Tyr Asn Val
Leu Pro Gln Gly Trp Lys Gly Ser Pro Ala Ile Phe Gln Ser Ser Met Thr Thr Val Lys Ala Ala Cys
Trp Trp Ala Gly Ile Lys Gln Glu Phe Gly Ile Pro Tyr Asn Pro Gln Ser Gln Gly Val Val Glu Ser
Met Asn Lys Glu Leu Lys Lys Ile Ile Gly Gln Val Arg Asp Gln Ala Glu His Leu Lys Thr Ala Val
Gln Met Ala Val Phe Ile His Asn Phe Lys Arg Lys Gly Gly Ile Gly Gly Tyr Ser Ala Gly Glu Arg
Ile Val Asp Ile Ile Asn Val Ser Thr Val Gln Cys Thr His Gly Ile Arg Pro Val Val Ser Thr Gln Leu
Leu Leu Asn Gly Ser Leu Ala Glu Glu Lys Arg Arg Val Val Gln Arg Glu Lys Arg Ala Val Gly
Ile Gly Ala Met Phe Leu Gly Phe Leu Gly Ala Ala Gly Ser Thr Met Gly Ala Ala Ser Ile Thr Leu
Thr Val Gln Ala Arg Gln Leu Leu Ser Gly Ile Val Gln Gln Gln Asn Asn Leu Leu Arg Ala Ile Glu
Ala Gln Gln His Leu Leu Gln Leu Thr Val Trp Gly Ile Lys Gln Leu Gln Ala Arg Val Leu Ala Val
Glu Arg Tyr Leu Lys Asp Gln Gln Leu Leu Gly Ile Trp Gly Cys Ser Gly Lys Leu Ile Cys Thr Thr
Val Ala Lys Glu Ile Val Ala Ser Cys Asp Lys Cys Gln Leu Lys Gly Glu Ala Met His Gly Gln Val
Asp Cys Ser Pro Gly Ile Trp Gln Leu Asp Cys Thr His Leu Glu Gly Lys Ile Ile Leu Val Ala Val
His Val Ala Ser Gly Tyr Ile Glu Ala Glu Val Ile Pro Ala Glu Thr Gly Gln Glu Thr Ala Tyr Phe
Leu Leu Lys Leu Ala Gly Arg Trp Pro Val Lys Thr Leu Trp Val Thr Val Tyr Tyr Gly Val Pro Val
Trp Lys Glu Ala Ala Phe Pro Gln Ile Thr Leu Trp Gln Arg Pro Leu Val Thr Ile Lys Ile Gly Gly
Gln Leu Lys Glu Ala Leu Leu Asp Thr Gly Ala Asp Asp Thr Val Leu Glu Glu Met Asn Leu Pro
Gly Arg Trp Lys Pro Lys Met Ile Gly Gly Ile Gly Gly Phe Ile Lys Val Arg Gln Tyr Asp Gln Ala
Ala Ala Ala His Asn Val Trp Ala Thr His Ala Cys Val Pro Thr Asp Pro Asn Pro Gln Glu Ala Ile
Thr Lys Ile Gln Asn Phe Arg Val Tyr Tyr Arg Asp Ser Arg Asp Pro Leu Trp Lys Gly Pro Ala Lys
Leu Leu Trp Lys Gly Glu Gly Ala Val Val Ile Gln Asp Asn Ser Asp Ile Lys Val Val Pro Arg Arg
Lys Ala Lys Ile Ile Arg Asp Tyr Gly Lys Gln Met Ala Gly Asp Asp Cys Val Ala Ser Arg Gln Asp
Glu Asp Pro Lys Phe Lys Leu Pro Ile Gln Lys Glu Thr Trp Glu Thr Trp Trp Thr Glu Tyr Trp Gln
Ala Thr Trp Ile Pro Glu Trp Glu Phe Val Asn Thr Pro Pro Leu Val Lys Leu Trp Tyr Gln Leu Glu
Lys Glu Pro Ile Val Gly Ala Glu Thr Phe Tyr Val Asp Gly Ala Ala Asn Arg Glu Thr Lys Ala Ala
Lys Glu Lys Val Tyr Leu Ala Trp Val Pro Ala His Lys Gly Ile Gly Gly Asn Glu Gln Val Asp Lys
Leu Val Ser Trp Gly Phe Thr Thr Pro Asp Lys Lys His Gln Lys Glu Pro Pro Phe Leu Trp Met
Gly Tyr Glu Leu His Pro Asp Lys Trp Thr Val Gln Pro Ile Val Leu Pro Glu Lys Asp Ser Trp Thr
Val Asn Asp Ile Gln Lys Leu Val Gly Lys Leu Asn Trp Ala Ser Gln Ile Tyr Pro Gly Ile Lys Val
Ile Val Ile Tyr Gln Tyr Met Asp Asp Leu Tyr Val Gly Ser Asp Leu Glu Ile Gly Gln His Arg Met
Arg Asp Asn Trp Arg Ser Glu Leu Tyr Lys Tyr Lys Val Val
(SEQ ID NO: 365)
Met Asn Pro Ser Ala Ala Val Ile Phe Cys Leu Ile Leu Leu Gly Leu
Ser Gly Thr Gln Gly Ile Leu Asp Met Ala Gln Pro Val Gly Ile Asn Thr Ser Thr Thr Cys Cys Tyr
Arg Phe Ile Asn Lys Lys Ile Pro Lys Gln Arg Leu Glu Ser Tyr Arg Arg Thr Thr Ser Ser His Cys
Pro Arg Glu Ala Val Ile Phe Lys Thr Lys Leu Asp Lys Glu Ile Cys Ala Asp Pro Thr Gln Lys Trp
Val Gln Asp Phe Met Lys His Leu Asp Lys Lys Thr Gln Thr Pro Lys Leu Ala Ser Ala Gly Ala
Gly Thr Val Leu Val Gly Pro Thr Pro Val Asn Ile Ile Gly Arg Asn Leu Leu Thr Gln Ile Gly Cys
Thr Leu Asn Phe Pro Ile Ser Pro Ile Glu Thr Val Pro Val Lys Leu Lys Pro Gly Met Asp Gly Pro
Lys Val Lys Gln Trp Pro Leu Thr Glu Glu Lys Ile Lys Ala Leu Val Glu Ile Cys Thr Glu Met Glu
Lys Glu Gly Lys Ile Ser Lys Ile Gly Pro Glu Asn Pro Tyr Asn Thr Pro Val Phe Ala Ile Lys Lys
Lys Asp Ser Thr Lys Trp Arg Lys Leu Val Asp Phe Arg Glu Leu Asn Lys Arg Thr Gln Asp Phe
Trp Glu Val Gln Leu Gly Ile Pro His Pro Ala Gly Leu Lys Lys Lys Lys Ser Val Thr Val Leu Asp
Val Gly Asp Ala Tyr Phe Ser Val Pro Leu Asp Lys Asp Phe Arg Lys Tyr Thr Ala Phe Thr Ile Pro
Ser Ile Asn Asn Glu Thr Pro Gly Ile Arg Tyr Gln Tyr Asn Val Leu Pro Gln Gly Trp Lys Gly Ser
Pro Ala Ile Phe Gln Ser Ser Met Thr Thr Val Lys Ala Ala Cys Trp Trp Ala Gly Ile Lys Gln Glu
Phe Gly Ile Pro Tyr Asn Pro Gln Ser Gln Gly Val Val Glu Ser Met Asn Lys Glu Leu Lys Lys Ile
Ile Gly Gln Val Arg Asp Gln Ala Glu His Leu Lys Thr Ala Val Gln Met Ala Val Phe Ile His Asn
Phe Lys Arg Lys Gly Gly Ile Gly Gly Tyr Ser Ala Gly Glu Arg Ile Val Asp Ile Ile Asn Val Ser
Thr Val Gln Cys Thr His Gly Ile Arg Pro Val Val Ser Thr Gln Leu Leu Leu Asn Gly Ser Leu Ala
Glu Glu Lys Arg Arg Val Val Gln Arg Glu Lys Arg Ala Val Gly Ile Gly Ala Met Phe Leu Gly
Phe Leu Gly Ala Ala Gly Ser Thr Met Gly Ala Ala Ser Ile Thr Leu Thr Val Gln Ala Arg Gln Leu
Leu Ser Gly Ile Val Gln Gln Gln Asn Asn Leu Leu Arg Ala Ile Glu Ala Gln Gln His Leu Leu Gln
Leu Thr Val Trp Gly Ile Lys Gln Leu Gln Ala Arg Val Leu Ala Val Glu Arg Tyr Leu Lys Asp
Gln Gln Leu Leu Gly Ile Trp Gly Cys Ser Gly Lys Leu Ile Cys Thr Thr Val Ala Lys Glu Ile Val
Ala Ser Cys Asp Lys Cys Gln Leu Lys Gly Glu Ala Met His Gly Gln Val Asp Cys Ser Pro Gly
Ile Trp Gln Leu Asp Cys Thr His Leu Glu Gly Lys Ile Ile Leu Val Ala Val His Val Ala Ser Gly
Tyr Ile Glu Ala Glu Val Ile Pro Ala Glu Thr Gly Gln Glu Thr Ala Tyr Phe Leu Leu Lys Leu Ala
Gly Arg Trp Pro Val Lys Thr Leu Trp Val Thr Val Tyr Tyr Gly Val Pro Val Trp Lys Glu Ala Ala
Phe Pro Gln Ile Thr Leu Trp Gln Arg Pro Leu Val Thr Ile Lys Ile Gly Gly Gln Leu Lys Glu Ala
Leu Leu Asp Thr Gly Ala Asp Asp Thr Val Leu Glu Glu Met Asn Leu Pro Gly Arg Trp Lys Pro
Lys Met Ile Gly Gly Ile Gly Gly Phe Ile Lys Val Arg Gln Tyr Asp Gln Ala Ala Ala Ala His Asn
Val Trp Ala Thr His Ala Cys Val Pro Thr Asp Pro Asn Pro Gln Glu Ala Ile Thr Lys Ile Gln Asn
Phe Arg Val Tyr Tyr Arg Asp Ser Arg Asp Pro Leu Trp Lys Gly Pro Ala Lys Leu Leu Trp Lys
Gly Glu Gly Ala Val Val Ile Gln Asp Asn Ser Asp Ile Lys Val Val Pro Arg Arg Lys Ala Lys Ile
Ile Arg Asp Tyr Gly Lys Gln Met Ala Gly Asp Asp Cys Val Ala Ser Arg Gln Asp Glu Asp Pro
Lys Phe Lys Leu Pro Ile Gln Lys Glu Thr Trp Glu Thr Trp Trp Thr Glu Tyr Trp Gln Ala Thr Trp
Ile Pro Glu Trp Glu Phe Val Asn Thr Pro Pro Leu Val Lys Leu Trp Tyr Gln Leu Glu Lys Glu Pro
Ile Val Gly Ala Glu Thr Phe Tyr Val Asp Gly Ala Ala Asn Arg Glu Thr Lys Ala Ala Lys Glu Lys
Val Tyr Leu Ala Trp Val Pro Ala His Lys Gly Ile Gly Gly Asn Glu Gln Val Asp Lys Leu Val Ser
Trp Gly Phe Thr Thr Pro Asp Lys Lys His Gln Lys Glu Pro Pro Phe Leu Trp Met Gly Tyr Glu
Leu His Pro Asp Lys Trp Thr Val Gln Pro Ile Val Leu Pro Glu Lys Asp Ser Trp Thr Val Asn Asp
Ile Gln Lys Leu Val Gly Lys Leu Asn Trp Ala Ser Gln Ile Tyr Pro Gly Ile Lys Val Ile Val Ile Tyr
Gln Tyr Met Asp Asp Leu Tyr Val Gly Ser Asp Leu Glu Ile Gly Gln His Arg Met Arg Asp Asn
Trp Arg Ser Glu Leu Tyr Lys Tyr Lys Val Val
(SEQ ID NO: 366)
Met Arg Lys Ala Ala Val Ser His Trp Gln Gln Gln Ser Tyr Leu Asp
Ser Gly Ile His Ser Gly Ala Thr Thr Thr Ala Pro Ser Leu Ser Gly Thr Val Leu Val Gly Pro Thr
Pro Val Asn Ile Ile Gly Arg Asn Leu Leu Thr Gln Ile Gly Cys Thr Leu Asn Phe Pro Ile Ser Pro
Ile Glu Thr Val Pro Val Lys Leu Lys Pro Gly Met Asp Gly Pro Lys Val Lys Gln Trp Pro Leu Thr
Glu Glu Lys Ile Lys Ala Leu Val Glu Ile Cys Thr Glu Met Glu Lys Glu Gly Lys Ile Ser Lys Ile
Gly Pro Glu Asn Pro Tyr Asn Thr Pro Val Phe Ala Ile Lys Lys Lys Asp Ser Thr Lys Trp Arg Lys
Leu Val Asp Phe Arg Glu Leu Asn Lys Arg Thr Gln Asp Phe Trp Glu Val Gln Leu Gly Ile Pro
His Pro Ala Gly Leu Lys Lys Lys Lys Ser Val Thr Val Leu Asp Val Gly Asp Ala Tyr Phe Ser Val
Pro Leu Asp Lys Asp Phe Arg Lys Tyr Thr Ala Phe Thr Ile Pro Ser Ile Asn Asn Glu Thr Pro Gly
Ile Arg Tyr Gln Tyr Asn Val Leu Pro Gln Gly Trp Lys Gly Ser Pro Ala Ile Phe Gln Ser Ser Met
Thr Thr Val Lys Ala Ala Cys Trp Trp Ala Gly Ile Lys Gln Glu Phe Gly Ile Pro Tyr Asn Pro Gln
Ser Gln Gly Val Val Glu Ser Met Asn Lys Glu Leu Lys Lys Ile Ile Gly Gln Val Arg Asp Gln Ala
Glu His Leu Lys Thr Ala Val Gln Met Ala Val Phe Ile His Asn Phe Lys Arg Lys Gly Gly Ile Gly
Gly Tyr Ser Ala Gly Glu Arg Ile Val Asp Ile Ile Asn Val Ser Thr Val Gln Cys Thr His Gly Ile
Arg Pro Val Val Ser Thr Gln Leu Leu Leu Asn Gly Ser Leu Ala Glu Glu Lys Arg Arg Val Val
Gln Arg Glu Lys Arg Ala Val Gly Ile Gly Ala Met Phe Leu Gly Phe Leu Gly Ala Ala Gly Ser Thr
Met Gly Ala Ala Ser Ile Thr Leu Thr Val Gln Ala Arg Gln Leu Leu Ser Gly Ile Val Gln Gln Gln
Asn Asn Leu Leu Arg Ala Ile Glu Ala Gln Gln His Leu Leu Gln Leu Thr Val Trp Gly Ile Lys Gln
Leu Gln Ala Arg Val Leu Ala Val Glu Arg Tyr Leu Lys Asp Gln Gln Leu Leu Gly Ile Trp Gly
Cys Ser Gly Lys Leu Ile Cys Thr Thr Val Ala Lys Glu Ile Val Ala Ser Cys Asp Lys Cys Gln Leu
Lys Gly Glu Ala Met His Gly Gln Val Asp Cys Ser Pro Gly Ile Trp Gln Leu Asp Cys Thr His Leu
Glu Gly Lys Ile Ile Leu Val Ala Val His Val Ala Ser Gly Tyr Ile Glu Ala Glu Val Ile Pro Ala Glu
Thr Gly Gln Glu Thr Ala Tyr Phe Leu Leu Lys Leu Ala Gly Arg Trp Pro Val Lys Thr Leu Trp
Val Thr Val Tyr Tyr Gly Val Pro Val Trp Lys Glu Ala Ala Phe Pro Gln Ile Thr Leu Trp Gln Arg
Pro Leu Val Thr Ile Lys Ile Gly Gly Gln Leu Lys Glu Ala Leu Leu Asp Thr Gly Ala Asp Asp Thr
Val Leu Glu Glu Met Asn Leu Pro Gly Arg Trp Lys Pro Lys Met Ile Gly Gly Ile Gly Gly Phe Ile
Lys Val Arg Gln Tyr Asp Gln Ala Ala Ala Ala His Asn Val Trp Ala Thr His Ala Cys Val Pro Thr
Asp Pro Asn Pro Gln Glu Ala Ile Thr Lys Ile Gln Asn Phe Arg Val Tyr Tyr Arg Asp Ser Arg Asp
Pro Leu Trp Lys Gly Pro Ala Lys Leu Leu Trp Lys Gly Glu Gly Ala Val Val Ile Gln Asp Asn Ser
Asp Ile Lys Val Val Pro Arg Arg Lys Ala Lys Ile Ile Arg Asp Tyr Gly Lys Gln Met Ala Gly Asp
Asp Cys Val Ala Ser Arg Gln Asp Glu Asp Pro Lys Phe Lys Leu Pro Ile Gln Lys Glu Thr Trp
Glu Thr Trp Trp Thr Glu Tyr Trp Gln Ala Thr Trp Ile Pro Glu Trp Glu Phe Val Asn Thr Pro Pro
Leu Val Lys Leu Trp Tyr Gln Leu Glu Lys Glu Pro Ile Val Gly Ala Glu Thr Phe Tyr Val Asp Gly
Ala Ala Asn Arg Glu Thr Lys Ala Ala Lys Glu Lys Val Tyr Leu Ala Trp Val Pro Ala His Lys Gly
Ile Gly Gly Asn Glu Gln Val Asp Lys Leu Val Ser Trp Gly Phe Thr Thr Pro Asp Lys Lys His Gln
Lys Glu Pro Pro Phe Leu Trp Met Gly Tyr Glu Leu His Pro Asp Lys Trp Thr Val Gln Pro Ile Val
Leu Pro Glu Lys Asp Ser Trp Thr Val Asn Asp Ile Gln Lys Leu Val Gly Lys Leu Asn Trp Ala
Ser Gln Ile Tyr Pro Gly Ile Lys Val Ile Val Ile Tyr Gln Tyr Met Asp Asp Leu Tyr Val Gly Ser
Asp Leu Glu Ile Gly Gln His Arg Met Arg Asp Asn Trp Arg Ser Glu Leu Tyr Lys Tyr Lys Val
Val
(SEQ ID NO: 367)
Tyr Gln Tyr Asn Val Leu Pro Gln Gly Ala Ser Arg Glu Leu Glu Arg
Phe Ala Val Asn Pro Gly Leu Leu Trp Ile Ile Leu Gly Leu Asn Lys Ile Val Arg Met Tyr Ser Pro
Thr Ser Ile Ala Ala Arg Thr Leu Asn Ala Trp Val Lys Val Phe Leu Trp Met Gly Tyr Glu Leu His
Leu Thr Phe Gly Trp Cys Phe Lys Leu Pro Leu Trp Lys Gly Pro Ala Lys Leu Val Thr Val Tyr
Tyr Gly Val Pro Val Ala Ala Leu Leu Trp Lys Gly Glu Gly Ala Val Ala Ala Ala Lys Leu Val Gly
Lys Leu Asn Trp Ala Lys Leu Leu Trp Lys Gly Glu Gly Ala Thr Leu Asn Phe Pro Ile Ser Pro Ile
Trp Gln Ala Thr Trp Ile Pro Glu Trp Lys Ala Ala Cys Trp Trp Ala Gly Ile Arg Gln Ala Asn Phe
Leu Gly Lys Ile Trp Pro Ser His Lys Gly Arg Asn Val Trp Ala Thr His Ala Cys Val Ala Ala Glu
Met Met Thr Ala Cys Gln Gly Val Ser Thr Val Gln Cys Thr His Gly Ile Ala Ala Lys Gln Met Ala
Gly Asp Asp Cys Val Ala Ala Trp Gln Leu Asp Cys Thr His Leu Glu Tyr Lys Ala Ala Val Asp
Leu Ser His Phe Leu Arg Glu Lys Gly Gly Leu Glu Gly Ala Ala Tyr Tyr Met Asp Asp Leu Tyr
Val Gly Ser Gly Gln Val Asp Cys Ser Pro Gly Ile Ala Thr Leu Glu Glu Met Met Thr Ala Glu Leu
His Pro Asp Lys Trp Thr Val Trp Thr Val Asn Asp Ile Gln Lys Leu Gly Ile Trp Gly Cys Ser Gly
Lys Leu Thr Val Asn Asp Ile Gln Lys Leu Val Ile Val Thr Asp Ser Gln Tyr Ala Leu Tyr Val Asp
Arg Phe Tyr Lys Thr Leu Arg Ala Glu Gln Ala Ser Gln Glu Val Asp Leu Asn Thr Met Leu Asn
Thr Val Lys Leu Thr Pro Leu Cys Val Thr Leu Tyr Gln Tyr Met Asp Asp Leu Tyr Val Val Ile
Tyr Gln Tyr Met Asp Asp Leu
(SEQ ID NO: 368)
Met Trp Leu Gln Ser Leu Leu Leu Leu Gly Thr Val Ala Cys Ser Ile
Ser Val Tyr Gln Tyr Asn Val Leu Pro Gln Gly Ala Ser Arg Glu Leu Glu Arg Phe Ala Val Asn
Pro Gly Leu Leu Trp Ile Ile Leu Gly Leu Asn Lys Ile Val Arg Met Tyr Ser Pro Thr Ser Ile Ala
Ala Arg Thr Leu Asn Ala Trp Val Lys Val Phe Leu Trp Met Gly Tyr Glu Leu His Leu Thr Phe
Gly Trp Cys Phe Lys Leu Pro Leu Trp Lys Gly Pro Ala Lys Leu Val Thr Val Tyr Tyr Gly Val
Pro Val Ala Ala Leu Leu Trp Lys Gly Glu Gly Ala Val Ala Ala Ala Lys Leu Val Gly Lys Leu
Asn Trp Ala Lys Leu Leu Trp Lys Gly Glu Gly Ala Thr Leu Asn Phe Pro Ile Ser Pro Ile Trp Gln
Ala Thr Trp Ile Pro Glu Trp Lys Ala Ala Cys Trp Trp Ala Gly Ile Arg Gln Ala Asn Phe Leu Gly
Lys Ile Trp Pro Ser His Lys Gly Arg Asn Val Trp Ala Thr His Ala Cys Val Ala Ala Glu Met Met
Thr Ala Cys Gln Gly Val Ser Thr Val Gln Cys Thr His Gly Ile Ala Ala Lys Gln Met Ala Gly Asp
Asp Cys Val Ala Ala Trp Gln Leu Asp Cys Thr His Leu Glu Tyr Lys Ala Ala Val Asp Leu Ser
His Phe Leu Arg Glu Lys Gly Gly Leu Glu Gly Ala Ala Tyr Tyr Met Asp Asp Leu Tyr Val Gly
Ser Gly Gln Val Asp Cys Ser Pro Gly Ile Ala Thr Leu Glu Glu Met Met Thr Ala Glu Leu His Pro
Asp Lys Trp Thr Val Trp Thr Val Asn Asp Ile Gln Lys Leu Gly Ile Trp Gly Cys Ser Gly Lys Leu
Thr Val Asn Asp Ile Gln Lys Leu Val Ile Val Thr Asp Ser Gln Tyr Ala Leu Tyr Val Asp Arg Phe
Tyr Lys Thr Leu Arg Ala Glu Gln Ala Ser Gln Glu Val Asp Leu Asn Thr Met Leu Asn Thr Val
Lys Leu Thr Pro Leu Cys Val Thr Leu Tyr Gln Tyr Met Asp Asp Leu Tyr Val Val Ile Tyr Gln
Tyr Met Asp Asp Leu
(SEQ ID NO: 369)
Met Asp Ala Met Lys Arg Gly Leu Cys Cys Val Leu Leu Leu Cys
Gly Ala Val Phe Val Ser Ala Arg Tyr Gln Tyr Asn Val Leu Pro Gln Gly Ala Ser Arg Glu Leu
Glu Arg Phe Ala Val Asn Pro Gly Leu Leu Trp Ile Ile Leu Gly Leu Asn Lys Ile Val Arg Met Tyr
Ser Pro Thr Ser Ile Ala Ala Arg Thr Leu Asn Ala Trp Val Lys Val Phe Leu Trp Met Gly Tyr Glu
Leu His Leu Thr Phe Gly Trp Cys Phe Lys Leu Pro Leu Trp Lys Gly Pro Ala Lys Leu Val Thr
Val Tyr Tyr Gly Val Pro Val Ala Ala Leu Leu Trp Lys Gly Glu Gly Ala Val Ala Ala Ala Lys Leu
Val Gly Lys Leu Asn Trp Ala Lys Leu Leu Trp Lys Gly Glu Gly Ala Thr Leu Asn Phe Pro Ile
Ser Pro Ile Trp Gln Ala Thr Trp Ile Pro Glu Trp Lys Ala Ala Cys Trp Trp Ala Gly Ile Arg Gln
Ala Asn Phe Leu Gly Lys Ile Trp Pro Ser His Lys Gly Arg Asn Val Trp Ala Thr His Ala Cys Val
Ala Ala Glu Met Met Thr Ala Cys Gln Gly Val Ser Thr Val Gln Cys Thr His Gly Ile Ala Ala Lys
Gln Met Ala Gly Asp Asp Cys Val Ala Ala Trp Gln Leu Asp Cys Thr His Leu Glu Tyr Lys Ala
Ala Val Asp Leu Ser His Phe Leu Arg Glu Lys Gly Gly Leu Glu Gly Ala Ala Tyr Tyr Met Asp
Asp Leu Tyr Val Gly Ser Gly Gln Val Asp Cys Ser Pro Gly Ile Ala Thr Leu Glu Glu Met Met Thr
Ala Glu Leu His Pro Asp Lys Trp Thr Val Trp Thr Val Asn Asp Ile Gln Lys Leu Gly Ile Trp Gly
Cys Ser Gly Lys Leu Thr Val Asn Asp Ile Gln Lys Leu Val Ile Val Thr Asp Ser Gln Tyr Ala Leu
Tyr Val Asp Arg Phe Tyr Lys Thr Leu Arg Ala Glu Gln Ala Ser Gln Glu Val Asp Leu Asn Thr
Met Leu Asn Thr Val Lys Leu Thr Pro Leu Cys Val Thr Leu Tyr Gln Tyr Met Asp Asp Leu Tyr
Val Val Ile Tyr Gln Tyr Met Asp Asp Leu
(SEQ ID NO: 370)
Met Asn Pro Ser Ala Ala Val Ile Phe Cys Leu Ile Leu Leu Gly Leu
Ser Gly Thr Gln Gly Ile Leu Asp Met Ala Gln Pro Val Gly Ile Asn Thr Ser Thr Thr Cys Cys Tyr
Arg Phe Ile Asn Lys Lys Ile Pro Lys Gln Arg Leu Glu Ser Tyr Arg Arg Thr Thr Ser Ser His Cys
Pro Arg Glu Ala Val Ile Phe Lys Thr Lys Leu Asp Lys Glu Ile Cys Ala Asp Pro Thr Gln Lys Trp
Val Gln Asp Phe Met Lys His Leu Asp Lys Lys Thr Gln Thr Pro Lys Leu Ala Ser Ala Gly Ala
Tyr Gln Tyr Asn Val Leu Pro Gln Gly Ala Ser Arg Glu Leu Glu Arg Phe Ala Val Asn Pro Gly
Leu Leu Trp Ile Ile Leu Gly Leu Asn Lys Ile Val Arg Met Tyr Ser Pro Thr Ser Ile Ala Ala Arg
Thr Leu Asn Ala Trp Val Lys Val Phe Leu Trp Met Gly Tyr Glu Leu His Leu Thr Phe Gly Trp
Cys Phe Lys Leu Pro Leu Trp Lys Gly Pro Ala Lys Leu Val Thr Val Tyr Tyr Gly Val Pro Val Ala
Ala Leu Leu Trp Lys Gly Glu Gly Ala Val Ala Ala Ala Lys Leu Val Gly Lys Leu Asn Trp Ala
Lys Leu Leu Trp Lys Gly Glu Gly Ala Thr Leu Asn Phe Pro Ile Ser Pro Ile Trp Gln Ala Thr Trp
Ile Pro Glu Trp Lys Ala Ala Cys Trp Trp Ala Gly Ile Arg Gln Ala Asn Phe Leu Gly Lys Ile Trp
Pro Ser His Lys Gly Arg Asn Val Trp Ala Thr His Ala Cys Val Ala Ala Glu Met Met Thr Ala
Cys Gln Gly Val Ser Thr Val Gln Cys Thr His Gly Ile Ala Ala Lys Gln Met Ala Gly Asp Asp Cys
Val Ala Ala Trp Gln Leu Asp Cys Thr His Leu Glu Tyr Lys Ala Ala Val Asp Leu Ser His Phe
Leu Arg Glu Lys Gly Gly Leu Glu Gly Ala Ala Tyr Tyr Met Asp Asp Leu Tyr Val Gly Ser Gly
Gln Val Asp Cys Ser Pro Gly Ile Ala Thr Leu Glu Glu Met Met Thr Ala Glu Leu His Pro Asp Lys
Trp Thr Val Trp Thr Val Asn Asp Ile Gln Lys Leu Gly Ile Trp Gly Cys Ser Gly Lys Leu Thr Val
Asn Asp Ile Gln Lys Leu Val Ile Val Thr Asp Ser Gln Tyr Ala Leu Tyr Val Asp Arg Phe Tyr Lys
Thr Leu Arg Ala Glu Gln Ala Ser Gln Glu Val Asp Leu Asn Thr Met Leu Asn Thr Val Lys Leu
Thr Pro Leu Cys Val Thr Leu Tyr Gln Tyr Met Asp Asp Leu Tyr Val Val Ile Tyr Gln Tyr Met
Asp Asp Leu
(SEQ ID NO: 371)
Met Arg Lys Ala Ala Val Ser His Trp Gln Gln Gln Ser Tyr Leu Asp
Ser Gly Ile His Ser Gly Ala Thr Thr Thr Ala Pro Ser Leu Ser Tyr Gln Tyr Asn Val Leu Pro Gln
Gly Ala Ser Arg Glu Leu Glu Arg Phe Ala Val Asn Pro Gly Leu Leu Trp Ile Ile Leu Gly Leu Asn
Lys Ile Val Arg Met Tyr Ser Pro Thr Ser Ile Ala Ala Arg Thr Leu Asn Ala Trp Val Lys Val Phe
Leu Trp Met Gly Tyr Glu Leu His Leu Thr Phe Gly Trp Cys Phe Lys Leu Pro Leu Trp Lys Gly
Pro Ala Lys Leu Val Thr Val Tyr Tyr Gly Val Pro Val Ala Ala Leu Leu Trp Lys Gly Glu Gly Ala
Val Ala Ala Ala Lys Leu Val Gly Lys Leu Asn Trp Ala Lys Leu Leu Trp Lys Gly Glu Gly Ala
Thr Leu Asn Phe Pro Ile Ser Pro Ile Trp Gln Ala Thr Trp Ile Pro Glu Trp Lys Ala Ala Cys Trp
Trp Ala Gly Ile Arg Gln Ala Asn Phe Leu Gly Lys Ile Trp Pro Ser His Lys Gly Arg Asn Val Trp
Ala Thr His Ala Cys Val Ala Ala Glu Met Met Thr Ala Cys Gln Gly Val Ser Thr Val Gln Cys
Thr His Gly Ile Ala Ala Lys Gln Met Ala Gly Asp Asp Cys Val Ala Ala Trp Gln Leu Asp Cys
Thr His Leu Glu Tyr Lys Ala Ala Val Asp Leu Ser His Phe Leu Arg Glu Lys Gly Gly Leu Glu
Gly Ala Ala Tyr Tyr Met Asp Asp Leu Tyr Val Gly Ser Gly Gln Val Asp Cys Ser Pro Gly Ile Ala
Thr Leu Glu Glu Met Met Thr Ala Glu Leu His Pro Asp Lys Trp Thr Val Trp Thr Val Asn Asp
Ile Gln Lys Leu Gly Ile Trp Gly Cys Ser Gly Lys Leu Thr Val Asn Asp Ile Gln Lys Leu Val Ile
Val Thr Asp Ser Gln Tyr Ala Leu Tyr Val Asp Arg Phe Tyr Lys Thr Leu Arg Ala Glu Gln Ala
Ser Gln Glu Val Asp Leu Asn Thr Met Leu Asn Thr Val Lys Leu Thr Pro Leu Cys Val Thr Leu
Tyr Gln Tyr Met Asp Asp Leu Tyr Val Val Ile Tyr Gln Tyr Met Asp Asp Leu
(SEQ ID NO: 372)
Met Ala Pro Arg Ser Ala Arg Arg Pro Leu Leu Leu Leu Leu Leu Leu
Leu Leu Leu Gly Leu Met His Cys Ala Ser Ala Ala Met Phe Met Val Lys Asn Gly Asn Gly Thr
Ala Cys Ile Met Ala Asn Phe Ser Ala Ala Phe Ser Val Asn Tyr Asp Thr Lys Ser Gly Pro Lys Asn
Met Thr Leu Asp Leu Pro Ser Asp Ala Thr Val Val Leu Asn Arg Ser Ser Cys Gly Lys Glu Asn
Thr Ser Asp Pro Ser Leu Val Ile Ala Phe Gly Arg Gly His Thr Leu Thr Leu Asn Phe Thr Arg Asn
Ala Thr Arg Tyr Ser Val Gln Leu Met Ser Phe Val Tyr Asn Leu Ser Asp Thr His Leu Phe Pro
Asn Ala Ser Ser Lys Glu Ile Lys Thr Val Glu Ser Ile Thr Asp Ile Arg Ala Asp Ile Asp Lys Lys
Tyr Arg Cys Val Ser Gly Thr Gln Val His Met Asn Asn Val Thr Val Thr Leu His Asp Ala Thr Ile
Gln Ala Tyr Leu Ser Asn Ser Ser Phe Ser Arg Gly Glu Thr Arg Cys Glu Gln Asp Arg Pro Ser Pro
Thr Thr Ala Pro Pro Ala Pro Pro Ser Pro Ser Pro Ser Pro Val Pro Lys Ser Pro Ser Val Asp Lys
Tyr Asn Val Ser Gly Thr Asn Gly Thr Cys Leu Leu Ala Ser Met Gly Leu Gln Leu Asn Leu Thr
Tyr Glu Arg Lys Asp Asn Thr Thr Val Thr Arg Leu Leu Asn Ile Asn Pro Asn Lys Thr Ser Ala
Ser Gly Ser Cys Gly Ala His Leu Val Thr Leu Glu Leu His Ser Glu Gly Thr Thr Val Leu Leu Phe
Gln Phe Gly Met Asn Ala Ser Ser Ser Arg Phe Phe Leu Gln Gly Ile Gln Leu Asn Thr Leu Leu
Pro Asp Ala Arg Asp Pro Ala Phe Lys Ala Ala Asn Gly Ser Leu Arg Ala Leu Gln Ala Thr Val
Gly Asn Ser Tyr Lys Cys Asn Ala Glu Glu His Val Arg Val Thr Lys Ala Phe Ser Val Asn Ile Phe
Lys Val Trp Val Gln Ala Phe Lys Val Glu Gly Gly Gln Phe Gly Ser Val Glu Glu Cys Leu Leu
Asp Glu Asn Ser Leu Glu Asp Ile Arg Thr Leu Asn Ala Trp Val Lys Val Arg Glu Lys Arg Asp
Leu Asn Thr Met Leu Asn Thr Val Arg Glu Lys Arg Trp Ile Ile Leu Gly Leu Asn Lys Ile Arg Glu
Lys Arg Tyr Val Asp Arg Phe Tyr Lys Thr Leu Arg Glu Lys Arg Ala Thr Leu Glu Glu Met Met
Thr Ala Arg Glu Lys Arg Glu Met Met Thr Ala Cys Gln Gly Val Arg Glu Lys Arg Thr Leu Asn
Phe Pro Ile Ser Pro Ile Arg Glu Lys Arg Tyr Gln Tyr Asn Val Leu Pro Gln Gly Arg Glu Lys Arg
Val Ile Tyr Gln Tyr Met Asp Asp Leu Arg Glu Lys Arg Tyr Gln Tyr Met Asp Asp Leu Tyr Val
Arg Glu Lys Arg Tyr Met Asp Asp Leu Tyr Val Gly Ser Arg Glu Lys Arg Phe Leu Trp Met Gly
Tyr Glu Leu His Arg Glu Lys Arg Glu Leu His Pro Asp Lys Trp Thr Val Arg Glu Lys Arg Trp
Thr Val Asn Asp Ile Gln Lys Leu Arg Glu Lys Arg Thr Val Asn Asp Ile Gln Lys Leu Val Arg
Glu Lys Arg Lys Leu Val Gly Lys Leu Asn Trp Ala Arg Glu Lys Arg Trp Gln Ala Thr Trp Ile
Pro Glu Trp Arg Glu Lys Arg Ile Val Thr Asp Ser Gln Tyr Ala Leu Arg Glu Lys Arg Gly Gln Val
Asp Cys Ser Pro Gly Ile Arg Glu Lys Arg Trp Gln Leu Asp Cys Thr His Leu Glu Arg Glu Lys
Arg Lys Ala Ala Cys Trp Trp Ala Gly Ile Arg Glu Lys Arg Pro Leu Trp Lys Gly Pro Ala Lys Leu
Arg Glu Lys Arg Lys Leu Leu Trp Lys Gly Glu Gly Ala Arg Glu Lys Arg Leu Leu Trp Lys Gly
Glu Gly Ala Val Arg Glu Lys Arg Lys Gln Met Ala Gly Asp Asp Cys Val Arg Glu Lys Arg Val
Thr Val Tyr Tyr Gly Val Pro Val Arg Glu Lys Arg Asn Val Trp Ala Thr His Ala Cys Val Arg
Glu Lys Arg Lys Leu Thr Pro Leu Cys Val Thr Leu Arg Glu Lys Arg Ser Thr Val Gln Cys Thr
His Gly Ile Arg Glu Lys Arg Gly Ile Trp Gly Cys Ser Gly Lys Leu Arg Glu Lys Arg Leu Thr Phe
Gly Trp Cys Phe Lys Leu Arg Glu Lys Arg Ala Ser Arg Glu Leu Glu Arg Phe Ala Val Asn Pro
Gly Leu Leu Arg Glu Lys Arg Trp Ile Ile Leu Gly Leu Asn Lys Ile Val Arg Met Tyr Ser Pro Thr
Ser Ile Arg Glu Lys Arg Tyr Val Asp Arg Phe Tyr Lys Thr Leu Arg Ala Glu Gln Ala Ser Gln Glu
Val Arg Glu Lys Arg Arg Gln Ala Asn Phe Leu Gly Lys Ile Trp Pro Ser His Lys Gly Arg Arg
Glu Lys Arg Tyr Lys Ala Ala Val Asp Leu Ser His Phe Leu Arg Glu Lys Gly Gly Leu Glu Gly
Gly Ser Glu Phe Thr Leu Ile Pro Ile Ala Val Gly Gly Ala Leu Ala Gly Leu Val Ile Val Leu Ile
Ala Tyr Leu Val Gly Arg Lys Arg Ser His Ala Gly Tyr Gln Thr Ile
(SEQ ID NO: 373)
Ala Ser Arg Glu Leu Glu Arg Phe Ala Val Asn Pro Gly Leu Leu Gln
Leu Lys Gly Glu Ala Met His Gly Gln Val Asp Cys Ser Pro Gly Ile Trp Gln Leu Asp Cys Thr
His Leu Cys Ser Ala Thr Glu Lys Leu Trp Val Thr Val Tyr Tyr Gly Val Pro Val Trp Lys Glu Ala
Thr Thr Thr Leu Pro Val Gly Glu Ile Tyr Lys Arg Trp Ile Ile Leu Gly Leu Asn Lys Ile Val Arg
Met Tyr Ser Pro Thr Ser Ile Asn Asn Glu Thr Pro Gly Ile Arg Tyr Gln Tyr Asn Val Leu Pro Gln
Gly Trp Lys Gly Ser Pro Ala Ile Phe Ala Ala Pro Leu Trp Lys Gly Pro Ala Lys Leu Leu Trp Lys
Gly Glu Gly Ala Val Val Ile Gln Asp Asn Ser Asp Ile Ala Ala Ile Val Leu Pro Glu Lys Asp Ser
Trp Thr Val Asn Asp Ile Gln Lys Leu Val Gly Lys Leu Asn Trp Ala Ser Val Tyr Tyr Arg Asp Ser
Arg Asp Pro Leu Trp Lys Gly Pro Ala Lys Leu Leu Trp Lys Gly Glu Gly Ala Val Ser Asn Phe
Thr Ser Thr Thr Val Lys Ala Ala Cys Trp Trp Ala Gly Ile Lys Gln Glu Phe Gly Ile Pro Tyr Val
Leu Pro Glu Lys Asp Ser Trp Thr Val Asn Asp Ile Gln Lys Leu Val Gly Lys Leu Asn Trp Ala
Ser Gln Met Val His Gln Ala Ile Ser Pro Arg Thr Leu Asn Ala Trp Val Lys Val Val Glu Glu Lys
Ala Phe Ser Pro Ala Ala Trp Glu Thr Trp Trp Thr Glu Tyr Trp Gln Ala Thr Trp Ile Pro Glu Trp
Glu Phe Val Asn Thr Pro Pro Leu Gln Asp Ser Gly Leu Glu Val Asn Ile Val Thr Asp Ser Gln Tyr
Ala Leu Gly Ile Ile Gln Ala Gln Pro Asp Ser Trp Thr Val Asn Asp Ile Gln Lys Leu Val Gly Lys
Leu Asn Trp Ala Ser Gln Ile Tyr Pro Gly Ile Lys Asn Pro Asp Ile Val Ile Tyr Gln Tyr Met Asp
Asp Leu Tyr Val Gly Ser Asp Leu Glu Ile Gly Gln His Arg Ala Ala Asp Pro Leu Trp Lys Gly Pro
Ala Lys Leu Leu Trp Lys Gly Glu Gly Ala Val Val Ile Gln Asp Asn Ser Asp Arg Gln Ala Asn
Phe Leu Gly Lys Ile Trp Pro Ser His Lys Gly Arg Ala Ala Pro Pro Phe Leu Trp Met Gly Tyr Glu
Leu His Pro Asp Lys Trp Thr Val Gln Pro Ile Val Leu Pro Glu Lys Phe Arg Lys Gln Asn Pro Asp
Ile Val Ile Tyr Gln Tyr Met Asp Asp Leu Tyr Val Gly Ser Asp Leu Glu Ile Asn Leu Leu Thr Gln
Ile Gly Cys Thr Leu Asn Phe Pro Ile Ser Pro Ile Glu Thr Val Pro Val Lys Leu Lys Gly Pro Lys
Glu Pro Phe Arg Asp Tyr Val Asp Arg Phe Tyr Lys Thr Leu Arg Ala Glu Gln Ala Ser Gln Glu
Val Tyr Leu Lys Asp Gln Gln Leu Leu Gly Ile Trp Gly Cys Ser Gly Lys Leu Ile Cys Thr Thr Ala
Val Pro Trp Ala Ala Ala Lys Lys His Gln Lys Glu Pro Pro Phe Leu Trp Met Gly Tyr Glu Leu His
Pro Asp Lys Trp Thr Val Gln Pro Gly Thr Gly Pro Cys Thr Asn Val Ser Thr Val Gln Cys Thr His
Gly Ile Arg Pro Val Val Ser Thr Gln Leu Lys Gln Asn Pro Asp Ile Val Ile Tyr Gln Tyr Met Asp
Asp Leu Tyr Val Gly Ser Asp Leu Glu Ile Gly Gln Leu Gly Pro Ala Ala Thr Leu Glu Glu Met
Met Thr Ala Cys Gln Gly Val Gly Gly Pro Gly His Lys Ala Arg Asp Gln Ser Leu Lys Pro Cys
Val Lys Leu Thr Pro Leu Cys Val Thr Leu Asn Cys Thr Asp Leu Arg Asn Thr Gly Pro Gly Ile
Arg Tyr Pro Leu Leu Thr Phe Gly Trp Cys Phe Lys Leu Pro Val Glu Pro Glu Lys Val Glu Ala
Lys Ile Ile Arg Asp Tyr Gly Lys Gln Met Ala Gly Asp Asp Cys Val Ala Ser Arg Gln Asp Glu
Asp Ile Leu Lys Ala Leu Gly Pro Ala Ala Thr Leu Glu Glu Met Met Thr Ala Cys Gln Gly Val
Gly Gly Pro Gly Tyr Lys Ala Ala Val Asp Leu Ser His Phe Leu Arg Glu Lys Gly Gly Leu Glu
Gly Ala Ala Tyr Ala Ala Lys Ala Tyr Asp Thr Glu Val His Asn Val Trp Ala Thr His Ala Cys Val
Pro Thr Asp Pro Asn Pro Gln Glu Ala Ala Ala Leu Ser Glu Gly Ala Thr Pro Gln Asp Leu Asn
Thr Met Leu Asn Thr Val Gly Gly His Gln Ala Ala Met Gln Gln Val Asp Cys Ser Pro Gly Ile Trp
Gln Leu Asp Cys Thr His Leu Glu Gly Lys Ile Ile Leu Val Ala Val
(SEQ ID NO: 374)
Met Trp Leu Gln Ser Leu Leu Leu Leu Gly Thr Val Ala Cys Ser Ile
Ser Val Ala Ser Arg Glu Leu Glu Arg Phe Ala Val Asn Pro Gly Leu Leu Gln Leu Lys Gly Glu
Ala Met His Gly Gln Val Asp Cys Ser Pro Gly Ile Trp Gln Leu Asp Cys Thr His Leu Cys Ser Ala
Thr Glu Lys Leu Trp Val Thr Val Tyr Tyr Gly Val Pro Val Trp Lys Glu Ala Thr Thr Thr Leu Pro
Val Gly Glu Ile Tyr Lys Arg Trp Ile Ile Leu Gly Leu Asn Lys Ile Val Arg Met Tyr Ser Pro Thr
Ser Ile Asn Asn Glu Thr Pro Gly Ile Arg Tyr Gln Tyr Asn Val Leu Pro Gln Gly Trp Lys Gly Ser
Pro Ala Ile Phe Ala Ala Pro Leu Trp Lys Gly Pro Ala Lys Leu Leu Trp Lys Gly Glu Gly Ala Val
Val Ile Gln Asp Asn Ser Asp Ile Ala Ala Ile Val Leu Pro Glu Lys Asp Ser Trp Thr Val Asn Asp
Ile Gln Lys Leu Val Gly Lys Leu Asn Trp Ala Ser Val Tyr Tyr Arg Asp Ser Arg Asp Pro Leu Trp
Lys Gly Pro Ala Lys Leu Leu Trp Lys Gly Glu Gly Ala Val Ser Asn Phe Thr Ser Thr Thr Val Lys
Ala Ala Cys Trp Trp Ala Gly Ile Lys Gln Glu Phe Gly Ile Pro Tyr Val Leu Pro Glu Lys Asp Ser
Trp Thr Val Asn Asp Ile Gln Lys Leu Val Gly Lys Leu Asn Trp Ala Ser Gln Met Val His Gln Ala
Ile Ser Pro Arg Thr Leu Asn Ala Trp Val Lys Val Val Glu Glu Lys Ala Phe Ser Pro Ala Ala Trp
Glu Thr Trp Trp Thr Glu Tyr Trp Gln Ala Thr Trp Ile Pro Glu Trp Glu Phe Val Asn Thr Pro Pro
Leu Gln Asp Ser Gly Leu Glu Val Asn Ile Val Thr Asp Ser Gln Tyr Ala Leu Gly Ile Ile Gln Ala
Gln Pro Asp Ser Trp Thr Val Asn Asp Ile Gln Lys Leu Val Gly Lys Leu Asn Trp Ala Ser Gln Ile
Tyr Pro Gly Ile Lys Asn Pro Asp Ile Val Ile Tyr Gln Tyr Met Asp Asp Leu Tyr Val Gly Ser Asp
Leu Glu Ile Gly Gln His Arg Ala Ala Asp Pro Leu Trp Lys Gly Pro Ala Lys Leu Leu Trp Lys Gly
Glu Gly Ala Val Val Ile Gln Asp Asn Ser Asp Arg Gln Ala Asn Phe Leu Gly Lys Ile Trp Pro Ser
His Lys Gly Arg Ala Ala Pro Pro Phe Leu Trp Met Gly Tyr Glu Leu His Pro Asp Lys Trp Thr
Val Gln Pro Ile Val Leu Pro Glu Lys Phe Arg Lys Gln Asn Pro Asp Ile Val Ile Tyr Gln Tyr Met
Asp Asp Leu Tyr Val Gly Ser Asp Leu Glu Ile Asn Leu Leu Thr Gln Ile Gly Cys Thr Leu Asn
Phe Pro Ile Ser Pro Ile Glu Thr Val Pro Val Lys Leu Lys Gly Pro Lys Glu Pro Phe Arg Asp Tyr
Val Asp Arg Phe Tyr Lys Thr Leu Arg Ala Glu Gln Ala Ser Gln Glu Val Tyr Leu Lys Asp Gln
Gln Leu Leu Gly Ile Trp Gly Cys Ser Gly Lys Leu Ile Cys Thr Thr Ala Val Pro Trp Ala Ala Ala
Lys Lys His Gln Lys Glu Pro Pro Phe Leu Trp Met Gly Tyr Glu Leu His Pro Asp Lys Trp Thr
Val Gln Pro Gly Thr Gly Pro Cys Thr Asn Val Ser Thr Val Gln Cys Thr His Gly Ile Arg Pro Val
Val Ser Thr Gln Leu Lys Gln Asn Pro Asp Ile Val Ile Tyr Gln Tyr Met Asp Asp Leu Tyr Val Gly
Ser Asp Leu Glu Ile Gly Gln Leu Gly Pro Ala Ala Thr Leu Glu Glu Met Met Thr Ala Cys Gln
Gly Val Gly Gly Pro Gly His Lys Ala Arg Asp Gln Ser Leu Lys Pro Cys Val Lys Leu Thr Pro
Leu Cys Val Thr Leu Asn Cys Thr Asp Leu Arg Asn Thr Gly Pro Gly Ile Arg Tyr Pro Leu Leu
Thr Phe Gly Trp Cys Phe Lys Leu Pro Val Glu Pro Glu Lys Val Glu Ala Lys Ile Ile Arg Asp Tyr
Gly Lys Gln Met Ala Gly Asp Asp Cys Val Ala Ser Arg Gln Asp Glu Asp Ile Leu Lys Ala Leu
Gly Pro Ala Ala Thr Leu Glu Glu Met Met Thr Ala Cys Gln Gly Val Gly Gly Pro Gly Tyr Lys
Ala Ala Val Asp Leu Ser His Phe Leu Arg Glu Lys Gly Gly Leu Glu Gly Ala Ala Tyr Ala Ala
Lys Ala Tyr Asp Thr Glu Val His Asn Val Trp Ala Thr His Ala Cys Val Pro Thr Asp Pro Asn Pro
Gln Glu Ala Ala Ala Leu Ser Glu Gly Ala Thr Pro Gln Asp Leu Asn Thr Met Leu Asn Thr Val
Gly Gly His Gln Ala Ala Met Gln Gln Val Asp Cys Ser Pro Gly Ile Trp Gln Leu Asp Cys Thr His
Leu Glu Gly Lys Ile Ile Leu Val Ala Val
(SEQ ID NO: 375)
Met Asp Ala Met Lys Arg Gly Leu Cys Cys Val Leu Leu Leu Cys
Gly Ala Val Phe Val Ser Ala Arg Ala Ser Arg Glu Leu Glu Arg Phe Ala Val Asn Pro Gly Leu
Leu Gln Leu Lys Gly Glu Ala Met His Gly Gln Val Asp Cys Ser Pro Gly Ile Trp Gln Leu Asp
Cys Thr His Leu Cys Ser Ala Thr Glu Lys Leu Trp Val Thr Val Tyr Tyr Gly Val Pro Val Trp Lys
Glu Ala Thr Thr Thr Leu Pro Val Gly Glu Ile Tyr Lys Arg Trp Ile Ile Leu Gly Leu Asn Lys Ile
Val Arg Met Tyr Ser Pro Thr Ser Ile Asn Asn Glu Thr Pro Gly Ile Arg Tyr Gln Tyr Asn Val Leu
Pro Gln Gly Trp Lys Gly Ser Pro Ala Ile Phe Ala Ala Pro Leu Trp Lys Gly Pro Ala Lys Leu Leu
Trp Lys Gly Glu Gly Ala Val Val Ile Gln Asp Asn Ser Asp Ile Ala Ala Ile Val Leu Pro Glu Lys
Asp Ser Trp Thr Val Asn Asp Ile Gln Lys Leu Val Gly Lys Leu Asn Trp Ala Ser Val Tyr Tyr Arg
Asp Ser Arg Asp Pro Leu Trp Lys Gly Pro Ala Lys Leu Leu Trp Lys Gly Glu Gly Ala Val Ser
Asn Phe Thr Ser Thr Thr Val Lys Ala Ala Cys Trp Trp Ala Gly Ile Lys Gln Glu Phe Gly Ile Pro
Tyr Val Leu Pro Glu Lys Asp Ser Trp Thr Val Asn Asp Ile Gln Lys Leu Val Gly Lys Leu Asn
Trp Ala Ser Gln Met Val His Gln Ala Ile Ser Pro Arg Thr Leu Asn Ala Trp Val Lys Val Val Glu
Glu Lys Ala Phe Ser Pro Ala Ala Trp Glu Thr Trp Trp Thr Glu Tyr Trp Gln Ala Thr Trp Ile Pro
Glu Trp Glu Phe Val Asn Thr Pro Pro Leu Gln Asp Ser Gly Leu Glu Val Asn Ile Val Thr Asp Ser
Gln Tyr Ala Leu Gly Ile Ile Gln Ala Gln Pro Asp Ser Trp Thr Val Asn Asp Ile Gln Lys Leu Val
Gly Lys Leu Asn Trp Ala Ser Gln Ile Tyr Pro Gly Ile Lys Asn Pro Asp Ile Val Ile Tyr Gln Tyr
Met Asp Asp Leu Tyr Val Gly Ser Asp Leu Glu Ile Gly Gln His Arg Ala Ala Asp Pro Leu Trp
Lys Gly Pro Ala Lys Leu Leu Trp Lys Gly Glu Gly Ala Val Val Ile Gln Asp Asn Ser Asp Arg
Gln Ala Asn Phe Leu Gly Lys Ile Trp Pro Ser His Lys Gly Arg Ala Ala Pro Pro Phe Leu Trp Met
Gly Tyr Glu Leu His Pro Asp Lys Trp Thr Val Gln Pro Ile Val Leu Pro Glu Lys Phe Arg Lys Gln
Asn Pro Asp Ile Val Ile Tyr Gln Tyr Met Asp Asp Leu Tyr Val Gly Ser Asp Leu Glu Ile Asn Leu
Leu Thr Gln Ile Gly Cys Thr Leu Asn Phe Pro Ile Ser Pro Ile Glu Thr Val Pro Val Lys Leu Lys
Gly Pro Lys Glu Pro Phe Arg Asp Tyr Val Asp Arg Phe Tyr Lys Thr Leu Arg Ala Glu Gln Ala
Ser Gln Glu Val Tyr Leu Lys Asp Gln Gln Leu Leu Gly Ile Trp Gly Cys Ser Gly Lys Leu Ile Cys
Thr Thr Ala Val Pro Trp Ala Ala Ala Lys Lys His Gln Lys Glu Pro Pro Phe Leu Trp Met Gly Tyr
Glu Leu His Pro Asp Lys Trp Thr Val Gln Pro Gly Thr Gly Pro Cys Thr Asn Val Ser Thr Val Gln
Cys Thr His Gly Ile Arg Pro Val Val Ser Thr Gln Leu Lys Gln Asn Pro Asp Ile Val Ile Tyr Gln
Tyr Met Asp Asp Leu Tyr Val Gly Ser Asp Leu Glu Ile Gly Gln Leu Gly Pro Ala Ala Thr Leu
Glu Glu Met Met Thr Ala Cys Gln Gly Val Gly Gly Pro Gly His Lys Ala Arg Asp Gln Ser Leu
Lys Pro Cys Val Lys Leu Thr Pro Leu Cys Val Thr Leu Asn Cys Thr Asp Leu Arg Asn Thr Gly
Pro Gly Ile Arg Tyr Pro Leu Leu Thr Phe Gly Trp Cys Phe Lys Leu Pro Val Glu Pro Glu Lys Val
Glu Ala Lys Ile Ile Arg Asp Tyr Gly Lys Gln Met Ala Gly Asp Asp Cys Val Ala Ser Arg Gln Asp
Glu Asp Ile Leu Lys Ala Leu Gly Pro Ala Ala Thr Leu Glu Glu Met Met Thr Ala Cys Gln Gly
Val Gly Gly Pro Gly Tyr Lys Ala Ala Val Asp Leu Ser His Phe Leu Arg Glu Lys Gly Gly Leu
Glu Gly Ala Ala Tyr Ala Ala Lys Ala Tyr Asp Thr Glu Val His Asn Val Trp Ala Thr His Ala Cys
Val Pro Thr Asp Pro Asn Pro Gln Glu Ala Ala Ala Leu Ser Glu Gly Ala Thr Pro Gln Asp Leu
Asn Thr Met Leu Asn Thr Val Gly Gly His Gln Ala Ala Met Gln Gln Val Asp Cys Ser Pro Gly
Ile Trp Gln Leu Asp Cys Thr His Leu Glu Gly Lys Ile Ile Leu Val Ala Val
(SEQ ID NO: 376)
Met Asn Pro Ser Ala Ala Val Ile Phe Cys Leu Ile Leu Leu Gly Leu
Ser Gly Thr Gln Gly Ile Leu Asp Met Ala Gln Pro Val Gly Ile Asn Thr Ser Thr Thr Cys Cys Tyr
Arg Phe Ile Asn Lys Lys Ile Pro Lys Gln Arg Leu Glu Ser Tyr Arg Arg Thr Thr Ser Ser His Cys
Pro Arg Glu Ala Val Ile Phe Lys Thr Lys Leu Asp Lys Glu Ile Cys Ala Asp Pro Thr Gln Lys Trp
Val Gln Asp Phe Met Lys His Leu Asp Lys Lys Thr Gln Thr Pro Lys Leu Ala Ser Ala Gly Ala
Ala Ser Arg Glu Leu Glu Arg Phe Ala Val Asn Pro Gly Leu Leu Gln Leu Lys Gly Glu Ala Met
His Gly Gln Val Asp Cys Ser Pro Gly Ile Trp Gln Leu Asp Cys Thr His Leu Cys Ser Ala Thr Glu
Lys Leu Trp Val Thr Val Tyr Tyr Gly Val Pro Val Trp Lys Glu Ala Thr Thr Thr Leu Pro Val Gly
Glu Ile Tyr Lys Arg Trp Ile Ile Leu Gly Leu Asn Lys Ile Val Arg Met Tyr Ser Pro Thr Ser Ile Asn
Asn Glu Thr Pro Gly Ile Arg Tyr Gln Tyr Asn Val Leu Pro Gln Gly Trp Lys Gly Ser Pro Ala Ile
Phe Ala Ala Pro Leu Trp Lys Gly Pro Ala Lys Leu Leu Trp Lys Gly Glu Gly Ala Val Val Ile Gln
Asp Asn Ser Asp Ile Ala Ala Ile Val Leu Pro Glu Lys Asp Ser Trp Thr Val Asn Asp Ile Gln Lys
Leu Val Gly Lys Leu Asn Trp Ala Ser Val Tyr Tyr Arg Asp Ser Arg Asp Pro Leu Trp Lys Gly
Pro Ala Lys Leu Leu Trp Lys Gly Glu Gly Ala Val Ser Asn Phe Thr Ser Thr Thr Val Lys Ala Ala
Cys Trp Trp Ala Gly Ile Lys Gln Glu Phe Gly Ile Pro Tyr Val Leu Pro Glu Lys Asp Ser Trp Thr
Val Asn Asp Ile Gln Lys Leu Val Gly Lys Leu Asn Trp Ala Ser Gln Met Val His Gln Ala Ile Ser
Pro Arg Thr Leu Asn Ala Trp Val Lys Val Val Glu Glu Lys Ala Phe Ser Pro Ala Ala Trp Glu Thr
Trp Trp Thr Glu Tyr Trp Gln Ala Thr Trp Ile Pro Glu Trp Glu Phe Val Asn Thr Pro Pro Leu Gln
Asp Ser Gly Leu Glu Val Asn Ile Val Thr Asp Ser Gln Tyr Ala Leu Gly Ile Ile Gln Ala Gln Pro
Asp Ser Trp Thr Val Asn Asp Ile Gln Lys Leu Val Gly Lys Leu Asn Trp Ala Ser Gln Ile Tyr Pro
Gly Ile Lys Asn Pro Asp Ile Val Ile Tyr Gln Tyr Met Asp Asp Leu Tyr Val Gly Ser Asp Leu Glu
Ile Gly Gln His Arg Ala Ala Asp Pro Leu Trp Lys Gly Pro Ala Lys Leu Leu Trp Lys Gly Glu Gly
Ala Val Val Ile Gln Asp Asn Ser Asp Arg Gln Ala Asn Phe Leu Gly Lys Ile Trp Pro Ser His Lys
Gly Arg Ala Ala Pro Pro Phe Leu Trp Met Gly Tyr Glu Leu His Pro Asp Lys Trp Thr Val Gln
Pro Ile Val Leu Pro Glu Lys Phe Arg Lys Gln Asn Pro Asp Ile Val Ile Tyr Gln Tyr Met Asp Asp
Leu Tyr Val Gly Ser Asp Leu Glu Ile Asn Leu Leu Thr Gln Ile Gly Cys Thr Leu Asn Phe Pro Ile
Ser Pro Ile Glu Thr Val Pro Val Lys Leu Lys Gly Pro Lys Glu Pro Phe Arg Asp Tyr Val Asp Arg
Phe Tyr Lys Thr Leu Arg Ala Glu Gln Ala Ser Gln Glu Val Tyr Leu Lys Asp Gln Gln Leu Leu
Gly Ile Trp Gly Cys Ser Gly Lys Leu Ile Cys Thr Thr Ala Val Pro Trp Ala Ala Ala Lys Lys His
Gln Lys Glu Pro Pro Phe Leu Trp Met Gly Tyr Glu Leu His Pro Asp Lys Trp Thr Val Gln Pro
Gly Thr Gly Pro Cys Thr Asn Val Ser Thr Val Gln Cys Thr His Gly Ile Arg Pro Val Val Ser Thr
Gln Leu Lys Gln Asn Pro Asp Ile Val Ile Tyr Gln Tyr Met Asp Asp Leu Tyr Val Gly Ser Asp Leu
Glu Ile Gly Gln Leu Gly Pro Ala Ala Thr Leu Glu Glu Met Met Thr Ala Cys Gln Gly Val Gly
Gly Pro Gly His Lys Ala Arg Asp Gln Ser Leu Lys Pro Cys Val Lys Leu Thr Pro Leu Cys Val
Thr Leu Asn Cys Thr Asp Leu Arg Asn Thr Gly Pro Gly Ile Arg Tyr Pro Leu Leu Thr Phe Gly
Trp Cys Phe Lys Leu Pro Val Glu Pro Glu Lys Val Glu Ala Lys Ile Ile Arg Asp Tyr Gly Lys Gln
Met Ala Gly Asp Asp Cys Val Ala Ser Arg Gln Asp Glu Asp Ile Leu Lys Ala Leu Gly Pro Ala
Ala Thr Leu Glu Glu Met Met Thr Ala Cys Gln Gly Val Gly Gly Pro Gly Tyr Lys Ala Ala Val
Asp Leu Ser His Phe Leu Arg Glu Lys Gly Gly Leu Glu Gly Ala Ala Tyr Ala Ala Lys Ala Tyr
Asp Thr Glu Val His Asn Val Trp Ala Thr His Ala Cys Val Pro Thr Asp Pro Asn Pro Gln Glu Ala
Ala Ala Leu Ser Glu Gly Ala Thr Pro Gln Asp Leu Asn Thr Met Leu Asn Thr Val Gly Gly His
Gln Ala Ala Met Gln Gln Val Asp Cys Ser Pro Gly Ile Trp Gln Leu Asp Cys Thr His Leu Glu
Gly Lys Ile Ile Leu Val Ala Val
(SEQ ID NO: 377)
Met Arg Lys Ala Ala Val Ser His Trp Gln Gln Gln Ser Tyr Leu Asp
Ser Gly Ile His Ser Gly Ala Thr Thr Thr Ala Pro Ser Leu Ser Ala Ser Arg Glu Leu Glu Arg Phe
Ala Val Asn Pro Gly Leu Leu Gln Leu Lys Gly Glu Ala Met His Gly Gln Val Asp Cys Ser Pro
Gly Ile Trp Gln Leu Asp Cys Thr His Leu Cys Ser Ala Thr Glu Lys Leu Trp Val Thr Val Tyr Tyr
Gly Val Pro Val Trp Lys Glu Ala Thr Thr Thr Leu Pro Val Gly Glu Ile Tyr Lys Arg Trp Ile Ile
Leu Gly Leu Asn Lys Ile Val Arg Met Tyr Ser Pro Thr Ser Ile Asn Asn Glu Thr Pro Gly Ile Arg
Tyr Gln Tyr Asn Val Leu Pro Gln Gly Trp Lys Gly Ser Pro Ala Ile Phe Ala Ala Pro Leu Trp Lys
Gly Pro Ala Lys Leu Leu Trp Lys Gly Glu Gly Ala Val Val Ile Gln Asp Asn Ser Asp Ile Ala Ala
Ile Val Leu Pro Glu Lys Asp Ser Trp Thr Val Asn Asp Ile Gln Lys Leu Val Gly Lys Leu Asn Trp
Ala Ser Val Tyr Tyr Arg Asp Ser Arg Asp Pro Leu Trp Lys Gly Pro Ala Lys Leu Leu Trp Lys
Gly Glu Gly Ala Val Ser Asn Phe Thr Ser Thr Thr Val Lys Ala Ala Cys Trp Trp Ala Gly Ile Lys
Gln Glu Phe Gly Ile Pro Tyr Val Leu Pro Glu Lys Asp Ser Trp Thr Val Asn Asp Ile Gln Lys Leu
Val Gly Lys Leu Asn Trp Ala Ser Gln Met Val His Gln Ala Ile Ser Pro Arg Thr Leu Asn Ala Trp
Val Lys Val Val Glu Glu Lys Ala Phe Ser Pro Ala Ala Trp Glu Thr Trp Trp Thr Glu Tyr Trp Gln
Ala Thr Trp Ile Pro Glu Trp Glu Phe Val Asn Thr Pro Pro Leu Gln Asp Ser Gly Leu Glu Val Asn
Ile Val Thr Asp Ser Gln Tyr Ala Leu Gly Ile Ile Gln Ala Gln Pro Asp Ser Trp Thr Val Asn Asp
Ile Gln Lys Leu Val Gly Lys Leu Asn Trp Ala Ser Gln Ile Tyr Pro Gly Ile Lys Asn Pro Asp Ile
Val Ile Tyr Gln Tyr Met Asp Asp Leu Tyr Val Gly Ser Asp Leu Glu Ile Gly Gln His Arg Ala Ala
Asp Pro Leu Trp Lys Gly Pro Ala Lys Leu Leu Trp Lys Gly Glu Gly Ala Val Val Ile Gln Asp
Asn Ser Asp Arg Gln Ala Asn Phe Leu Gly Lys Ile Trp Pro Ser His Lys Gly Arg Ala Ala Pro Pro
Phe Leu Trp Met Gly Tyr Glu Leu His Pro Asp Lys Trp Thr Val Gln Pro Ile Val Leu Pro Glu Lys
Phe Arg Lys Gln Asn Pro Asp Ile Val Ile Tyr Gln Tyr Met Asp Asp Leu Tyr Val Gly Ser Asp Leu
Glu Ile Asn Leu Leu Thr Gln Ile Gly Cys Thr Leu Asn Phe Pro Ile Ser Pro Ile Glu Thr Val Pro
Val Lys Leu Lys Gly Pro Lys Glu Pro Phe Arg Asp Tyr Val Asp Arg Phe Tyr Lys Thr Leu Arg
Ala Glu Gln Ala Ser Gln Glu Val Tyr Leu Lys Asp Gln Gln Leu Leu Gly Ile Trp Gly Cys Ser Gly
Lys Leu Ile Cys Thr Thr Ala Val Pro Trp Ala Ala Ala Lys Lys His Gln Lys Glu Pro Pro Phe Leu
Trp Met Gly Tyr Glu Leu His Pro Asp Lys Trp Thr Val Gln Pro Gly Thr Gly Pro Cys Thr Asn
Val Ser Thr Val Gln Cys Thr His Gly Ile Arg Pro Val Val Ser Thr Gln Leu Lys Gln Asn Pro Asp
Ile Val Ile Tyr Gln Tyr Met Asp Asp Leu Tyr Val Gly Ser Asp Leu Glu Ile Gly Gln Leu Gly Pro
Ala Ala Thr Leu Glu Glu Met Met Thr Ala Cys Gln Gly Val Gly Gly Pro Gly His Lys Ala Arg
Asp Gln Ser Leu Lys Pro Cys Val Lys Leu Thr Pro Leu Cys Val Thr Leu Asn Cys Thr Asp Leu
Arg Asn Thr Gly Pro Gly Ile Arg Tyr Pro Leu Leu Thr Phe Gly Trp Cys Phe Lys Leu Pro Val Glu
Pro Glu Lys Val Glu Ala Lys Ile Ile Arg Asp Tyr Gly Lys Gln Met Ala Gly Asp Asp Cys Val Ala
Ser Arg Gln Asp Glu Asp Ile Leu Lys Ala Leu Gly Pro Ala Ala Thr Leu Glu Glu Met Met Thr
Ala Cys Gln Gly Val Gly Gly Pro Gly Tyr Lys Ala Ala Val Asp Leu Ser His Phe Leu Arg Glu
Lys Gly Gly Leu Glu Gly Ala Ala Tyr Ala Ala Lys Ala Tyr Asp Thr Glu Val His Asn Val Trp
Ala Thr His Ala Cys Val Pro Thr Asp Pro Asn Pro Gln Glu Ala Ala Ala Leu Ser Glu Gly Ala Thr
Pro Gln Asp Leu Asn Thr Met Leu Asn Thr Val Gly Gly His Gln Ala Ala Met Gln Gln Val Asp
Cys Ser Pro Gly Ile Trp Gln Leu Asp Cys Thr His Leu Glu Gly Lys Ile Ile Leu Val Ala Val
Ala Ala Ala
Ala Ala Tyr
Ala Ala Xaa, where Xaa is any amino acid
(SEQ ID NO: 381)
Arg Ala Lys Arg
(SEQ ID NO: 382)
Arg Glu Lys Arg
(SEQ ID NO: 383)
Arg Arg Lys Arg
(SEQ ID NO: 384)
Ala Thr Asn Phe Ser Leu Leu Lys Gln Ala Gly Asp Val Glu Glu Asn
Pro Gly Pro
(SEQ ID NO: 385)
Ala Pro Val Lys Gln Thr Leu Asn Phe Asp Leu Leu Lys Leu Ala Gly
Asp Val Glu Ser Asn Pro Gly Pro
(SEQ ID NO: 386)
Arg Ala Lys Arg Ala Pro Val Lys Gln Thr Leu Asn Phe Asp Leu Leu
Lys Leu Ala Gly Asp Val Glu Ser Asn Pro Gly Pro
(SEQ ID NO: 387)
Gln Cys Thr Asn Tyr Ala Leu Leu Lys Leu Ala Gly Asp Val Glu Ser
Asn Pro Gly Pro
(SEQ ID NO: 388)
Glu Gly Arg Gly Ser Leu Leu Thr Cys Gly Asp Val Glu Glu Asn Pro
Gly Pro
(SEQ ID NO: 389)
Val His Ala Gly Pro Ile Ala
(SEQ ID NO: 390)
Val His Ala Gly Pro Val Ala
(SEQ ID NO: 391)
Gly Ala Leu Asp Ile
(SEQ ID NO: 392)
Gly Ala Leu Asp Leu
(SEQ ID NO: 393)
Met Trp Leu Gln Ser Leu Leu Leu Leu Gly Thr Val Ala Cys Ser Ile
Ser Val
(SEQ ID NO: 394)
Met Asp Ala Met Lys Arg Gly Leu Cys Cys Val Leu Leu Leu Cys
Gly Ala Val Phe Val Ser Ala Arg
(SEQ ID NO: 395)
Met His Arg Arg Arg Ser Arg Ser Cys Arg Glu Asp Gln Lys Pro Val
(SEQ ID NO: 396)
Lys Trp Val Thr Phe Ile Ser Leu Leu Phe Leu Phe Ser Ser Ala Tyr
Ser
(SEQ ID NO: 397)
Met Arg Lys Ala Ala Val Ser His Trp Gln Gln Gln Ser Tyr Leu Asp
Ser Gly Ile His Ser Gly Ala Thr Thr Thr Ala Pro Ser Leu Ser
(SEQ ID NO: 398)
Met Asn Pro Ser Ala Ala Val Ile Phe Cys Leu Ile Leu Leu Gly Leu
Ser Gly Thr Gln Gly Ile Leu Asp Met Ala Gln Pro Val Gly Ile Asn Thr Ser Thr Thr Cys Cys Tyr
Arg Phe Ile Asn Lys Lys Ile Pro Lys Gln Arg Leu Glu Ser Tyr Arg Arg Thr Thr Ser Ser His Cys
Pro Arg Glu Ala Val Ile Phe Lys Thr Lys Leu Asp Lys Glu Ile Cys Ala Asp Pro Thr Gln Lys Trp
Val Gln Asp Phe Met Lys His Leu Asp Lys Lys Thr Gln Thr Pro Lys Leu Ala Ser Ala Gly Ala
(SEQ ID NO: 399)
Met Ala Pro Arg Ser Ala Arg Arg Pro Leu Leu Leu Leu Leu Leu Leu
Leu Leu Leu Gly Leu Met His Cys Ala Ser Ala Ala Met Phe Met Val Lys Asn Gly Asn Gly Thr
Ala Cys Ile Met Ala Asn Phe Ser Ala Ala Phe Ser Val Asn Tyr Asp Thr Lys Ser Gly Pro Lys Asn
Met Thr Leu Asp Leu Pro Ser Asp Ala Thr Val Val Leu Asn Arg Ser Ser Cys Gly Lys Glu Asn
Thr Ser Asp Pro Ser Leu Val Ile Ala Phe Gly Arg Gly His Thr Leu Thr Leu Asn Phe Thr Arg Asn
Ala Thr Arg Tyr Ser Val Gln Leu Met Ser Phe Val Tyr Asn Leu Ser Asp Thr His Leu Phe Pro
Asn Ala Ser Ser Lys Glu Ile Lys Thr Val Glu Ser Ile Thr Asp Ile Arg Ala Asp Ile Asp Lys Lys
Tyr Arg Cys Val Ser Gly Thr Gln Val His Met Asn Asn Val Thr Val Thr Leu His Asp Ala Thr Ile
Gln Ala Tyr Leu Ser Asn Ser Ser Phe Ser Arg Gly Glu Thr Arg Cys Glu Gln Asp Arg Pro Ser Pro
Thr Thr Ala Pro Pro Ala Pro Pro Ser Pro Ser Pro Ser Pro Val Pro Lys Ser Pro Ser Val Asp Lys
Tyr Asn Val Ser Gly Thr Asn Gly Thr Cys Leu Leu Ala Ser Met Gly Leu Gln Leu Asn Leu Thr
Tyr Glu Arg Lys Asp Asn Thr Thr Val Thr Arg Leu Leu Asn Ile Asn Pro Asn Lys Thr Ser Ala
Ser Gly Ser Cys Gly Ala His Leu Val Thr Leu Glu Leu His Ser Glu Gly Thr Thr Val Leu Leu Phe
Gln Phe Gly Met Asn Ala Ser Ser Ser Arg Phe Phe Leu Gln Gly Ile Gln Leu Asn Thr Leu Leu
Pro Asp Ala Arg Asp Pro Ala Phe Lys Ala Ala Asn Gly Ser Leu Arg Ala Leu Gln Ala Thr Val
Gly Asn Ser Tyr Lys Cys Asn Ala Glu Glu His Val Arg Val Thr Lys Ala Phe Ser Val Asn Ile Phe
Lys Val Trp Val Gln Ala Phe Lys Val Glu Gly Gly Gln Phe Gly Ser Val Glu Glu Cys Leu Leu
Asp Glu Asn Ser Leu Glu Asp Ile
(SEQ ID NO: 400)
Met Gln Ile Phe Val Lys Thr Leu Thr Gly Lys Thr Ile Thr Leu Glu
Val Glu Pro Ser Asp Thr Ile Glu Asn Val Lys Ala Lys Ile Gln Asp Lys Glu Gly Ile Pro Pro Asp
Gln Gln Arg Leu Ile Phe Ala Gly Lys Gln Leu Glu Asp Gly Arg Thr Leu Ser Asp Tyr Asn Ile Gln
Lys Glu Ser Thr Leu His Leu Val Leu Arg Leu Arg Gly Gly
(SEQ ID NO: 401)
Met Leu Leu Ser Val Pro Leu Leu Leu Gly Leu Leu Gly Leu Ala Val
Ala
(SEQ ID NO: 402)
Met Lys Cys Leu Leu Tyr Leu Ala Phe Leu Phe Ile Gly Val Asn Cys
(SEQ ID NO: 403)
Met Arg Val Lys Glu Lys Tyr Gln His Leu Trp Arg Trp Gly Trp Arg
Trp Gly Thr Met Leu Leu Gly Met Leu Met Ile Cys Ser Ala Thr Glu Lys Leu Trp Val Thr Val
Tyr Tyr Gly Val Pro Val Trp Lys Glu Ala Thr Thr Thr Leu Phe Cys Ala Ser Asp Ala Lys Ala Tyr
Asp Thr Glu Val His Asn Val Trp Ala Thr His Ala Cys Val Pro Thr Asp Pro Asn Pro Gln Glu Val
Val Leu Val Asn Val Thr Glu Asn Phe Asn Met Trp Lys Asn Asp Met Val Glu Gln Met His Glu
Asp Ile Ile Ser Leu Trp Asp Gln Ser Leu Lys Pro Cys Val Lys Leu Thr Pro Leu Cys Val Ser Leu
Lys Cys Thr Asp Leu Lys Asn Asp Thr Asn Thr Asn Ser Ser Ser Gly Arg Met Ile Met Glu Lys
Gly Glu Ile Lys Asn Cys Ser Phe Asn Ile Ser Thr Ser Ile Arg Gly Lys Val Gln Lys Glu Tyr Ala
Phe Phe Tyr Lys Leu Asp Ile Ile Pro Ile Asp Asn Asp Thr Thr Ser Tyr Lys Leu Thr Ser Cys Asn
Thr Ser Val Ile Thr Gln Ala Cys Pro Lys Val Ser Phe Glu Pro Ile Pro Ile His Tyr Cys Ala Pro Ala
Gly Phe Ala Ile Leu Lys Cys Asn Asn Lys Thr Phe Asn Gly Thr Gly Pro Cys Thr Asn Val Ser
Thr Val Gln Cys Thr His Gly Ile Arg Pro Val Val Ser Thr Gln Leu Leu Leu Asn Gly Ser Leu Ala
Glu Glu Glu Val Val Ile Arg Ser Val Asn Phe Thr Asp Asn Ala Lys Thr Ile Ile Val Gln Leu Asn
Thr Ser Val Glu Ile Asn Cys Thr Arg Pro Asn Asn Asn Thr Arg Lys Arg Ile Arg Ile Gln Arg Gly
Pro Gly Arg Ala Phe Val Thr Ile Gly Lys Ile Gly Asn Met Arg Gln Ala His Cys Asn Ile Ser Arg
Ala Lys Trp Asn Asn Thr Leu Lys Gln Ile Ala Ser Lys Leu Arg Glu Gln Phe Gly Asn Asn Lys
Thr Ile Ile Phe Lys Gln Ser Ser Gly Gly Asp Pro Glu Ile Val Thr His Ser Phe Asn Cys Gly Gly
Glu Phe Phe Tyr Cys Asn Ser Thr Gln Leu Phe Asn Ser Thr Trp Phe Asn Ser Thr Trp Ser Thr Glu
Gly Ser Asn Asn Thr Glu Gly Ser Asp Thr Ile Thr Leu Pro Cys Arg Ile Lys Gln Ile Ile Asn Met
Trp Gln Lys Val Gly Lys Ala Met Tyr Ala Pro Pro Ile Ser Gly Gln Ile Arg Cys Ser Ser Asn Ile
Thr Gly Leu Leu Leu Thr Arg Asp Gly Gly Asn Ser Asn Asn Glu Ser Glu Ile Phe Arg Pro Gly
Gly Gly Asp Met Arg Asp Asn Trp Arg Ser Glu Leu Tyr Lys Tyr Lys Val Val Lys Ile Glu Pro
Leu Gly Val Ala Pro Thr Lys Ala Lys Arg Arg Val Val Gln Arg Glu Lys Arg Ala Val Gly Ile Gly
Ala Leu Phe Leu Gly Phe Leu Gly Ala Ala Gly Ser Thr Met Gly Ala Ala Ser Met Thr Leu Thr
Val Gln Ala Arg Gln Leu Leu Ser Gly Ile Val Gln Gln Gln Asn Asn Leu Leu Arg Ala Ile Glu Ala
Gln Gln His Leu Leu Gln Leu Thr Val Trp Gly Ile Lys Gln Leu Gln Ala Arg Ile Leu Ala Val Glu
Arg Tyr Leu Lys Asp Gln Gln Leu Leu Gly Ile Trp Gly Cys Ser Gly Lys Leu Ile Cys Thr Thr Ala
Val Pro Trp Asn Ala Ser Trp Ser Asn Lys Ser Leu Glu Gln Ile Trp Asn His Thr Thr Trp Met Glu
Trp Asp Arg Glu Ile Asn Asn Tyr Thr Ser Leu Ile His Ser Leu Ile Glu Glu Ser Gln Asn Gln Gln
Glu Lys Asn Glu Gln Glu Leu Leu Glu Leu Asp Lys Trp Ala Ser Leu Trp Asn Trp Phe Asn Ile
Thr Asn Trp Leu Trp Tyr Ile Lys Leu Phe Ile Met Ile Val Gly Gly Leu Val Gly Leu Arg Ile Val
Phe Ala Val Leu Ser Ile Val Asn Arg Val Arg Gln Gly Tyr Ser Pro Leu Ser Phe Gln Thr His Leu
Pro Thr Pro Arg Gly Pro Asp Arg Pro Glu Gly Ile Glu Glu Glu Gly Gly Glu Arg Asp Arg Asp
Arg Ser Ile Arg Leu Val Asn Gly Ser Leu Ala Leu Ile Trp Asp Asp Leu Arg Ser Leu Cys Leu Phe
Ser Tyr His Arg Leu Arg Asp Leu Leu Leu Ile Val Thr Arg Ile Val Glu Leu Leu Gly Arg Arg Gly
Trp Glu Ala Leu Lys Tyr Trp Trp Asn Leu Leu Gln Tyr Trp Ser Gln Glu Leu Lys Asn Ser Ala
Val Ser Leu Leu Asn Ala Thr Ala Ile Ala Val Ala Glu Gly Thr Asp Arg Val Ile Glu Val Val Gln
Gly Ala Cys Arg Ala Ile Arg His Ile Pro Arg Arg Ile Arg Gln Gly Leu Glu Arg Ile Leu Leu
(SEQ ID NO: 404)
Met Gly Ala Arg Ala Ser Val Leu Ser Gly Gly Glu Leu Asp Arg Trp
Glu Lys Ile Arg Leu Arg Pro Gly Gly Lys Lys Lys Tyr Lys Leu Lys His Ile Val Trp Ala Ser Arg
Glu Leu Glu Arg Phe Ala Val Asn Pro Gly Leu Leu Glu Thr Ser Glu Gly Cys Arg Gln Ile Leu
Gly Gln Leu Gln Pro Ser Leu Gln Thr Gly Ser Glu Glu Leu Arg Ser Leu Tyr Asn Thr Val Ala
Thr Leu Tyr Cys Val His Gln Arg Ile Glu Ile Lys Asp Thr Lys Glu Ala Leu Asp Lys Ile Glu Glu
Glu Gln Asn Lys Ser Lys Lys Lys Ala Gln Gln Ala Ala Ala Asp Thr Gly His Ser Asn Gln Val
Ser Gln Asn Tyr Pro Ile Val Gln Asn Ile Gln Gly Gln Met Val His Gln Ala Ile Ser Pro Arg Thr
Leu Asn Ala Trp Val Lys Val Val Glu Glu Lys Ala Phe Ser Pro Glu Val Ile Pro Met Phe Ser Ala
Leu Ser Glu Gly Ala Thr Pro Gln Asp Leu Asn Thr Met Leu Asn Thr Val Gly Gly His Gln Ala
Ala Met Gln Met Leu Lys Glu Thr Ile Asn Glu Glu Ala Ala Glu Trp Asp Arg Val His Pro Val
His Ala Gly Pro Ile Ala Pro Gly Gln Met Arg Glu Pro Arg Gly Ser Asp Ile Ala Gly Thr Thr Ser
Thr Leu Gln Glu Gln Ile Gly Trp Met Thr Asn Asn Pro Pro Ile Pro Val Gly Glu Ile Tyr Lys Arg
Trp Ile Ile Leu Gly Leu Asn Lys Ile Val Arg Met Tyr Ser Pro Thr Ser Ile Leu Asp Ile Arg Gln
Gly Pro Lys Glu Pro Phe Arg Asp Tyr Val Asp Arg Phe Tyr Lys Thr Leu Arg Ala Glu Gln Ala
Ser Gln Glu Val Lys Asn Trp Met Thr Glu Thr Leu Leu Val Gln Asn Ala Asn Pro Asp Cys Lys
Thr Ile Leu Lys Ala Leu Gly Pro Ala Ala Thr Leu Glu Glu Met Met Thr Ala Cys Gln Gly Val
Gly Gly Pro Gly His Lys Ala Arg Val Leu Ala Glu Ala Met Ser Gln Val Thr Asn Ser Ala Thr Ile
Met Met Gln Arg Gly Asn Phe Arg Asn Gln Arg Lys Ile Val Lys Cys Phe Asn Cys Gly Lys Glu
Gly His Thr Ala Arg Asn Cys Arg Ala Pro Arg Lys Lys Gly Cys Trp Lys Cys Gly Lys Glu Gly
His Gln Met Lys Asp Cys Thr Glu Arg Gln Ala Asn Phe Leu Gly Lys Ile Trp Pro Ser Tyr Lys
Gly Arg Pro Gly Asn Phe Leu Gln Ser Arg Pro Glu Pro Thr Ala Pro Pro Glu Glu Ser Phe Arg Ser
Gly Val Glu Thr Thr Thr Pro Pro Gln Lys Gln Glu Pro Ile Asp Lys Glu Leu Tyr Pro Leu Thr Ser
Leu Arg Ser Leu Phe Gly Asn Asp Pro Ser Ser Gln
(SEQ ID NO: 405)
Met Gly Gly Lys Trp Ser Lys Ser Ser Val Ile Gly Trp Pro Thr Val
Arg Glu Arg Met Arg Arg Ala Glu Pro Ala Ala Asp Arg Val Gly Ala Ala Ser Arg Asp Leu Glu
Lys His Gly Ala Ile Thr Ser Ser Asn Thr Ala Ala Thr Asn Ala Ala Cys Ala Trp Leu Glu Ala Gln
Glu Glu Glu Glu Val Gly Phe Pro Val Thr Pro Gln Val Pro Leu Arg Pro Met Thr Tyr Lys Ala Ala
Val Asp Leu Ser His Phe Leu Lys Glu Lys Gly Gly Leu Glu Gly Leu Ile His Ser Gln Arg Arg Gln
Asp Ile Leu Asp Leu Trp Ile Tyr His Thr Gln Gly Tyr Phe Pro Asp Trp Gln Asn Tyr Thr Pro Gly
Pro Gly Val Arg Tyr Pro Leu Thr Phe Gly Trp Cys Tyr Lys Leu Val Pro Val Glu Pro Asp Lys Ile
Glu Glu Ala Asn Lys Gly Glu Asn Thr Ser Leu Leu His Pro Val Ser Leu His Gly Met Asp Asp
Pro Glu Arg Glu Val Leu Glu Trp Arg Phe Asp Ser Arg Leu Ala Phe His His Val Ala Arg Glu
Leu His Pro Glu Tyr Phe Lys Asn Cys
(SEQ ID NO: 406)
Phe Phe Arg Glu Asp Leu Ala Phe Leu Gln Gly Lys Ala Arg Glu
Phe Ser Ser Glu Gln Thr Arg Ala Asn Ser Pro Thr Arg Arg Glu Leu Gln Val Trp Gly Arg Asp
Asn Asn Ser Pro Ser Glu Ala Gly Ala Asp Arg Gln Gly Thr Val Ser Phe Asn Phe Pro Gln Val
Thr Leu Trp Gln Arg Pro Leu Val Thr Ile Lys Ile Gly Gly Gln Leu Lys Glu Ala Leu Leu Asp
Thr Gly Ala Asp Asp Thr Val Leu Glu Glu Met Ser Leu Pro Gly Arg Trp Lys Pro Lys Met Ile
Gly Gly Ile Gly Gly Phe Ile Lys Val Arg Gln Tyr Asp Gln Ile Leu Ile Glu Ile Cys Gly His Lys
Ala Ile Gly Thr Val Leu Val Gly Pro Thr Pro Val Asn Ile Ile Gly Arg Asn Leu Leu Thr Gln Ile
Gly Cys Thr Leu Asn Phe Pro Ile Ser Pro Ile Glu Thr Val Pro Val Lys Leu Lys Pro Gly Met Asp
Gly Pro Lys Val Lys Gln Trp Pro Leu Thr Glu Glu Lys Ile Lys Ala Leu Val Glu Ile Cys Thr Glu
Met Glu Lys Glu Gly Lys Ile Ser Lys Ile Gly Pro Glu Asn Pro Tyr Asn Thr Pro Val Phe Ala Ile
Lys Lys Lys Asp Ser Thr Lys Trp Arg Lys Leu Val Asp Phe Arg Glu Leu Asn Lys Arg Thr Gln
Asp Phe Trp Glu Val Gln Leu Gly Ile Pro His Pro Ala Gly Leu Lys Lys Lys Lys Ser Val Thr
Val Leu Asp Val Gly Asp Ala Tyr Phe Ser Val Pro Leu Asp Glu Asp Phe Arg Lys Tyr Thr Ala
Phe Thr Ile Pro Ser Ile Asn Asn Glu Thr Pro Gly Ile Arg Tyr Gln Tyr Asn Val Leu Pro Gln Gly
Trp Lys Gly Ser Pro Ala Ile Phe Gln Ser Ser Met Thr Lys Ile Leu Glu Pro Phe Arg Lys Gln Asn
Pro Asp Ile Val Ile Tyr Gln Tyr Met Asp Asp Leu Tyr Val Gly Ser Asp Leu Glu Ile Gly Gln His
Arg Thr Lys Ile Glu Glu Leu Arg Gln His Leu Leu Arg Trp Gly Leu Thr Thr Pro Asp Lys Lys
His Gln Lys Glu Pro Pro Phe Leu Trp Met Gly Tyr Glu Leu His Pro Asp Lys Trp Thr Val Gln
Pro Ile Val Leu Pro Glu Lys Asp Ser Trp Thr Val Asn Asp Ile Gln Lys Leu Val Gly Lys Leu
Asn Trp Ala Ser Gln Ile Tyr Pro Gly Ile Lys Val Arg Gln Leu Cys Lys Leu Leu Arg Gly Thr
Lys Ala Leu Thr Glu Val Ile Pro Leu Thr Glu Glu Ala Glu Leu Glu Leu Ala Glu Asn Arg Glu
Ile Leu Lys Glu Pro Val His Gly Val Tyr Tyr Asp Pro Ser Lys Asp Leu Ile Ala Glu Ile Gln Lys
Gln Gly Gln Gly Gln Trp Thr Tyr Gln Ile Tyr Gln Glu Pro Phe Lys Asn Leu Lys Thr Gly Lys
Tyr Ala Arg Met Arg Gly Ala His Thr Asn Asp Val Lys Gln Leu Thr Glu Ala Val Gln Lys Ile
Thr Thr Glu Ser Ile Val Ile Trp Gly Lys Thr Pro Lys Phe Lys Leu Pro Ile Gln Lys Glu Thr Trp
Glu Thr Trp Trp Thr Glu Tyr Trp Gln Ala Thr Trp Ile Pro Glu Trp Glu Phe Val Asn Thr Pro Pro
Leu Val Lys Leu Trp Tyr Gln Leu Glu Lys Glu Pro Ile Val Gly Ala Glu Thr Phe Tyr Val Asp
Gly Ala Ala Asn Arg Glu Thr Lys Leu Gly Lys Ala Gly Tyr Val Thr Asn Arg Gly Arg Gln Lys
Val Val Thr Leu Thr Asp Thr Thr Asn Gln Lys Thr Glu Leu Gln Ala Ile Tyr Leu Ala Leu Gln
Asp Ser Gly Leu Glu Val Asn Ile Val Thr Asp Ser Gln Tyr Ala Leu Gly Ile Ile Gln Ala Gln Pro
Asp Gln Ser Glu Ser Glu Leu Val Asn Gln Ile Ile Glu Gln Leu Ile Lys Lys Glu Lys Val Tyr Leu
Ala Trp Val Pro Ala His Lys Gly Ile Gly Gly Asn Glu Gln Val Asp Lys Leu Val Ser Ala Gly Ile
Arg Lys Val Leu Phe Leu Asp Gly Ile Asp Lys Ala Gln Asp Glu His Glu Lys Tyr His Ser Asn
Trp Arg Ala Met Ala Ser Asp Phe Asn Leu Pro Pro Val Val Ala Lys Glu Ile Val Ala Ser Cys
Asp Lys Cys Gln Leu Lys Gly Glu Ala Met His Gly Gln Val Asp Cys Ser Pro Gly Ile Trp Gln
Leu Asp Cys Thr His Leu Glu Gly Lys Val Ile Leu Val Ala Val His Val Ala Ser Gly Tyr Ile Glu
Ala Glu Val Ile Pro Ala Glu Thr Gly Gln Glu Thr Ala Tyr Phe Leu Leu Lys Leu Ala Gly Arg
Trp Pro Val Lys Thr Ile His Thr Asp Asn Gly Ser Asn Phe Thr Gly Ala Thr Val Arg Ala Ala
Cys Trp Trp Ala Gly Ile Lys Gln Glu Phe Gly Ile Pro Tyr Asn Pro Gln Ser Gln Gly Val Val Glu
Ser Met Asn Lys Glu Leu Lys Lys Ile Ile Gly Gln Val Arg Asp Gln Ala Glu His Leu Lys Thr
Ala Val Gln Met Ala Val Phe Ile His Asn Phe Lys Arg Lys Gly Gly Ile Gly Gly Tyr Ser Ala Gly
Glu Arg Ile Val Asp Ile Ile Ala Thr Asp Ile Gln Thr Lys Glu Leu Gln Lys Gln Ile Thr Lys Ile
Gln Asn Phe Arg Val Tyr Tyr Arg Asp Ser Arg Asn Pro Leu Trp Lys Gly Pro Ala Lys Leu Leu
Trp Lys Gly Glu Gly Ala Val Val Ile Gln Asp Asn Ser Asp Ile Lys Val Val Pro Arg Arg Lys
Ala Lys Ile Ile Arg Asp Tyr Gly Lys Gln Met Ala Gly Asp Asp Cys Val Ala Ser Arg Gln Asp
Glu Asp
(SEQ ID NO: 407)
Thr Val Lys Ala Ala Cys Trp Trp Ala Gly Ile Lys Gln Glu Phe Gly
Ile Pro Tyr Asn Pro Gln Ser Gln Gly Val Val Glu Ser Met Asn Lys Glu Leu Lys Lys Ile Ile Gly
Gln Val Arg Asp Gln Ala Glu His Leu Lys Thr Ala Val Gln Met Ala Val Phe Ile His Asn Phe Lys
Arg Lys Gly Gly Ile Gly Gly Tyr Ser Ala Gly Glu Arg Ile Val Asp Ile Ile Ala Ile Thr Lys Ile Gln
Asn Phe Arg Val Tyr Tyr Arg Asp Ser Arg Asp Pro Leu Trp Lys Gly Pro Ala Lys Leu Leu Trp
Lys Gly Glu Gly Ala Val Val Ile Gln Asp Asn Ser Asp Ile Lys Val Val Pro Arg Arg Lys Ala Lys
Ile Ile Arg Asp Tyr Gly Lys Gln Met Ala Gly Asp Asp Cys Val Ala Ser Arg Gln Asp Glu Asp
Pro Lys Phe Lys Leu Pro Ile Gln Lys Glu Thr Trp Glu Thr Trp Trp Thr Glu Tyr Trp Gln Ala Thr
Trp Ile Pro Glu Trp Glu Phe Val Asn Thr Pro Pro Leu Val Lys Leu Trp Tyr Gln Leu Glu Lys Glu
Pro Ile Val Gly Ala Glu Thr Phe Tyr Val Asp Gly Ala Ala Asn Arg Glu Thr Lys Ala Ala Lys Glu
Lys Val Tyr Leu Ala Trp Val Pro Ala His Lys Gly Ile Gly Gly Asn Glu Gln Val Asp Lys Leu Val
Ser Trp Gly Phe Thr Thr Pro Asp Lys Lys His Gln Lys Glu Pro Pro Phe Leu Trp Met Gly Tyr Glu
Leu His Pro Asp Lys Trp Thr Val Gln Pro Ile Val Leu Pro Glu Lys Asp Ser Trp Thr Val Asn Asp
Ile Gln Lys Leu Val Gly Lys Leu Asn Trp Ala Ser Gln Ile Tyr Pro Gly Ile Lys Val Ile Val Ile Tyr
Gln Tyr Met Asp Asp Leu Tyr Val Gly Ser Asp Leu Glu Ile Gly Gln His Arg Val Ala Lys Glu Ile
Val Ala Ser Cys Asp Lys Cys Gln Leu Lys Gly Glu Ala Met His Gly Gln Val Asp Cys Ser Pro
Gly Ile Trp Gln Leu Asp Cys Thr His Leu Glu Gly Lys Ile Ile Leu Val Ala Val His Val Ala Ser
Gly Tyr Ile Glu Ala Glu Val Ile Pro Ala Glu Thr Gly Gln Glu Thr Ala Tyr Phe Leu Leu Lys Leu
Ala Gly Arg Trp Pro Val Lys Thr Ala Ala Phe Pro Gln Ile Thr Leu Trp Gln Arg Pro Leu Val Thr
Ile Lys Ile Gly Gly Gln Leu Lys Glu Ala Leu Leu Asp Thr Gly Ala Asp Asp Thr Val Leu Glu Glu
Met Asn Leu Pro Gly Arg Trp Lys Pro Lys Met Ile Gly Gly Ile Gly Gly Phe Ile Lys Val Arg Gln
Tyr Asp Gln Gly Thr Val Leu Val Gly Pro Thr Pro Val Asn Ile Ile Gly Arg Asn Leu Leu Thr Gln
Ile Gly Cys Thr Leu Asn Phe Pro Ile Ser Pro Ile Glu Thr Val Pro Val Lys Leu Lys Pro Gly Met
Asp Gly Pro Lys Val Lys Gln Trp Pro Leu Thr Glu Glu Lys Ile Lys Ala Leu Val Glu Ile Cys Thr
Glu Met Glu Lys Glu Gly Lys Ile Ser Lys Ile Gly Pro Glu Asn Pro Tyr Asn Thr Pro Val Phe Ala
Ile Lys Lys Lys Asp Ser Thr Lys Trp Arg Lys Leu Val Asp Phe Arg Glu Leu Asn Lys Arg Thr
Gln Asp Phe Trp Glu Val Gln Leu Gly Ile Pro His Pro Ala Gly Leu Lys Lys Lys Lys Ser Val Thr
Val Leu Asp Val Gly Asp Ala Tyr Phe Ser Val Pro Leu Asp Lys Asp Phe Arg Lys Tyr Thr Ala
Phe Thr Ile Pro Ser Ile Asn Asn Glu Thr Pro Gly Ile Arg Tyr Gln Tyr Asn Val Leu Pro Gln Gly
Trp Lys Gly Ser Pro Ala Ile Phe Gln Ser Ser Met Thr
(SEQ ID NO: 408)
Ala Val Lys Ala Ala Cys Trp Trp Ala Gly Val Lys Gln Glu Phe Gly
Ile Pro Tyr His Pro Gln Ser Gln Gly Val Val Glu Ser Met Asn Asn Glu Leu Lys Lys Ile Ile Gly
Gln Ile Arg Asp Gln Ala Glu Gln Leu Lys Thr Ala Val Gln Met Ala Val Leu Ile His Asn Phe Lys
Arg Lys Gly Gly Ile Gly Glu Tyr Ser Ala Gly Glu Arg Ile Ile Asp Ile Ile Val Ala Lys Glu Ile Val
Ala Cys Cys Asp Lys Cys Gln Leu Lys Gly Glu Ala Ile His Gly Gln Val Asp Cys Ser Pro Gly
Val Trp Gln Leu Asp Cys Thr His Leu Glu Gly Lys Val Ile Leu Val Ala Val His Val Ala Ser Gly
Tyr Met Glu Ala Glu Val Ile Pro Thr Glu Thr Gly Gln Glu Thr Ala Tyr Phe Ile Leu Lys Leu Ala
Gly Arg Trp Pro Val Thr Thr Trp Gly Leu Thr Thr Pro Asp Lys Lys His Gln Lys Asp Pro Pro Phe
Leu Trp Met Gly Tyr Glu Leu His Pro Asp Arg Trp Thr Val Gln Pro Ile Glu Leu Pro Glu Lys Glu
Ser Trp Thr Val Asn Asp Ile Gln Lys Leu Ile Gly Lys Leu Asn Trp Ala Ser Gln Ile Tyr Ala Gly
Ile Lys Val Ile Val Ile Tyr Gln Tyr Val Asp Asp Leu Tyr Val Gly Ser Asp Leu Glu Ile Glu Gln
His Arg Leu Pro Gln Ile Thr Leu Trp Gln Arg Pro Ile Val Thr Ile Lys Ile Gly Gly Gln Ile Lys Glu
Ala Leu Leu Asp Thr Gly Ala Asp Asp Thr Val Leu Glu Asp Met Asn Leu Pro Gly Lys Trp Lys
Pro Lys Met Ile Gly Gly Ile Gly Gly Phe Ile Lys Val Lys Gln Tyr Asp Gln Pro Lys Phe Arg Leu
Pro Ile Gln Lys Glu Thr Trp Asp Thr Trp Trp Thr Asp Tyr Trp Gln Ala Thr Trp Ile Pro Glu Trp
Glu Phe Thr Asn Thr Pro Pro Leu Val Lys Leu Trp Tyr Gln Leu Glu Thr Glu Pro Ile Ala Gly Val
Glu Thr Phe Tyr Val Asp Gly Ala Ser Asn Arg Glu Thr Lys Ala Ala Tyr Ala Ile Thr Lys Leu Gln
Asn Phe Arg Val Tyr Tyr Arg Asp Asn Arg Asp Pro Leu Trp Lys Gly Pro Ala Arg Leu Leu Trp
Lys Gly Glu Gly Ala Val Val Ile Gln Asp Asn Ser Glu Ile Lys Val Val Pro Arg Arg Lys Val Lys
Ile Ile Arg Asp Tyr Gly Lys Arg Met Ala Gly Asp Asp Cys Val Ala Gly Arg Gln Asp Glu Asp
Gly Thr Val Leu Ile Gly Pro Thr Pro Val Asn Ile Ile Gly Arg Asn Leu Leu Thr Gln Leu Gly Cys
Thr Leu Asn Phe Pro Ile Ser Pro Ile Asp Thr Val Pro Val Lys Leu Lys Pro Gly Met Asp Gly Pro
Arg Val Lys Gln Trp Pro Leu Thr Glu Glu Lys Ile Lys Ala Leu Ile Glu Ile Cys Thr Glu Met Glu
Lys Glu Gly Lys Ile Ser Arg Ile Gly Pro Glu Asn Pro Tyr Asn Thr Pro Ile Phe Ala Ile Lys Lys
Lys Asp Gly Thr Lys Trp Arg Lys Leu Val Asp Phe Arg Glu Leu Asn Lys Lys Thr Gln Asp Phe
Trp Glu Val Gln Leu Gly Ile Pro His Pro Ser Gly Leu Lys Lys Lys Lys Ser Val Thr Val Leu Asp
Ile Gly Asp Ala Tyr Phe Ser Val Pro Leu Asp Lys Glu Phe Arg Lys Tyr Thr Ala Phe Thr Val Pro
Ser Thr Asn Asn Glu Thr Pro Gly Val Arg Tyr Gln Tyr Asn Val Leu Pro Met Gly Trp Lys Gly
Ser Pro Ala Ile Phe Gln Cys Ser Met Thr Lys Glu Lys Ile Tyr Leu Ala Trp Val Pro Ala His Lys
Gly Ile Gly Gly Asn Glu Gln Ile Asp Lys Leu Val Ser
(SEQ ID NO: 409)
Thr Val Lys Ala Ala Cys Trp Trp Ala Gly Ile Lys Gln Glu Phe Gly
Ile Pro Tyr Asn Pro Gln Ser Gln Gly Val Val Glu Ser Met Asn Lys Glu Leu Lys Lys Ile Ile Gly
Gln Val Arg Asp Gln Ala Glu His Leu Lys Thr Ala Val Gln Met Ala Val Phe Ile His Asn Phe Lys
Arg Lys Gly Gly Ile Gly Gly Tyr Ser Ala Gly Glu Arg Ile Val Asp Ile Ile Val Ala Lys Glu Ile Val
Ala Ser Cys Asp Lys Cys Gln Leu Lys Gly Glu Ala Met His Gly Gln Val Asp Cys Ser Pro Gly
Ile Trp Gln Leu Asp Cys Thr His Leu Glu Gly Lys Ile Ile Leu Val Ala Val His Val Ala Ser Gly
Tyr Ile Glu Ala Glu Val Ile Pro Ala Glu Thr Gly Gln Glu Thr Ala Tyr Phe Leu Leu Lys Leu Ala
Gly Arg Trp Pro Val Lys Thr Ala Ile Thr Lys Ile Gln Asn Phe Arg Val Tyr Tyr Arg Asp Ser Arg
Asp Pro Leu Trp Lys Gly Pro Ala Lys Leu Leu Trp Lys Gly Glu Gly Ala Val Val Ile Gln Asp
Asn Ser Asp Ile Lys Val Val Pro Arg Arg Lys Ala Lys Ile Ile Arg Asp Tyr Gly Lys Gln Met Ala
Gly Asp Asp Cys Val Ala Ser Arg Gln Asp Glu Asp Pro Lys Phe Lys Leu Pro Ile Gln Lys Glu
Thr Trp Glu Thr Trp Trp Thr Glu Tyr Trp Gln Ala Thr Trp Ile Pro Glu Trp Glu Phe Val Asn Thr
Pro Pro Leu Val Lys Leu Trp Tyr Gln Leu Glu Lys Glu Pro Ile Val Gly Ala Glu Thr Phe Tyr Val
Asp Gly Ala Ala Asn Arg Glu Thr Lys Ala Ala Lys Glu Lys Val Tyr Leu Ala Trp Val Pro Ala
His Lys Gly Ile Gly Gly Asn Glu Gln Val Asp Lys Leu Val Ser Trp Gly Phe Thr Thr Pro Asp Lys
Lys His Gln Lys Glu Pro Pro Phe Leu Trp Met Gly Tyr Glu Leu His Pro Asp Lys Trp Thr Val
Gln Pro Ile Val Leu Pro Glu Lys Asp Ser Trp Thr Val Asn Asp Ile Gln Lys Leu Val Gly Lys Leu
Asn Trp Ala Ser Gln Ile Tyr Pro Gly Ile Lys Val Ala Ala Phe Pro Gln Ile Thr Leu Trp Gln Arg
Pro Leu Val Thr Ile Lys Ile Gly Gly Gln Leu Lys Glu Ala Leu Leu Asp Thr Gly Ala Asp Asp Thr
Val Leu Glu Glu Met Asn Leu Pro Gly Arg Trp Lys Pro Lys Met Ile Gly Gly Ile Gly Gly Phe Ile
Lys Val Arg Gln Tyr Asp Gln Gly Thr Val Leu Val Gly Pro Thr Pro Val Asn Ile Ile Gly Arg Asn
Leu Leu Thr Gln Ile Gly Cys Thr Leu Asn Phe Pro Ile Ser Pro Ile Glu Thr Val Pro Val Lys Leu
Lys Pro Gly Met Asp Gly Pro Lys Val Lys Gln Trp Pro Leu Thr Glu Glu Lys Ile Lys Ala Leu Val
Glu Ile Cys Thr Glu Met Glu Lys Glu Gly Lys Ile Ser Lys Ile Gly Pro Glu Asn Pro Tyr Asn Thr
Pro Val Phe Ala Ile Lys Lys Lys Asp Ser Thr Lys Trp Arg Lys Leu Val Asp Phe Arg Glu Leu
Asn Lys Arg Thr Gln Asp Phe Trp Glu Val Gln Leu Gly Ile Pro His Pro Ala Gly Leu Lys Lys Lys
Lys Ser Val Thr Val Leu Asp Val Gly Asp Ala Tyr Phe Ser Val Pro Leu Asp Lys Asp Phe Arg
Lys Tyr Thr Ala Phe Thr Ile Pro Ser Ile Asn Asn Glu Thr Pro Gly Ile Arg Tyr Gln Tyr Asn Val
Leu Pro Gln Gly Trp Lys Gly Ser Pro Ala Ile Phe Gln Ser Ser Met Thr
(SEQ ID NO: 410)
Ala Val Lys Ala Ala Cys Trp Trp Ala Gly Val Lys Gln Glu Phe Gly
Ile Pro Tyr His Pro Gln Ser Gln Gly Val Val Glu Ser Met Asn Asn Glu Leu Lys Lys Ile Ile Gly
Gln Ile Arg Asp Gln Ala Glu Gln Leu Lys Thr Ala Val Gln Met Ala Val Leu Ile His Asn Phe Lys
Arg Lys Gly Gly Ile Gly Glu Tyr Ser Ala Gly Glu Arg Ile Ile Asp Ile Ile Trp Gly Leu Thr Thr Pro
Asp Lys Lys His Gln Lys Asp Pro Pro Phe Leu Trp Met Gly Tyr Glu Leu His Pro Asp Arg Trp
Thr Val Gln Pro Ile Glu Leu Pro Glu Lys Glu Ser Trp Thr Val Asn Asp Ile Gln Lys Leu Ile Gly
Lys Leu Asn Trp Ala Ser Gln Ile Tyr Ala Gly Ile Lys Val Ala Ala Tyr Val Ala Lys Glu Ile Val
Ala Cys Cys Asp Lys Cys Gln Leu Lys Gly Glu Ala Ile His Gly Gln Val Asp Cys Ser Pro Gly
Val Trp Gln Leu Asp Cys Thr His Leu Glu Gly Lys Val Ile Leu Val Ala Val His Val Ala Ser Gly
Tyr Met Glu Ala Glu Val Ile Pro Thr Glu Thr Gly Gln Glu Thr Ala Tyr Phe Ile Leu Lys Leu Ala
Gly Arg Trp Pro Val Thr Thr Leu Pro Gln Ile Thr Leu Trp Gln Arg Pro Ile Val Thr Ile Lys Ile Gly
Gly Gln Ile Lys Glu Ala Leu Leu Asp Thr Gly Ala Asp Asp Thr Val Leu Glu Asp Met Asn Leu
Pro Gly Lys Trp Lys Pro Lys Met Ile Gly Gly Ile Gly Gly Phe Ile Lys Val Lys Gln Tyr Asp Gln
Pro Lys Phe Arg Leu Pro Ile Gln Lys Glu Thr Trp Asp Thr Trp Trp Thr Asp Tyr Trp Gln Ala Thr
Trp Ile Pro Glu Trp Glu Phe Thr Asn Thr Pro Pro Leu Val Lys Leu Trp Tyr Gln Leu Glu Thr Glu
Pro Ile Ala Gly Val Glu Thr Phe Tyr Val Asp Gly Ala Ser Asn Arg Glu Thr Lys Ala Ala Tyr Ala
Ile Thr Lys Leu Gln Asn Phe Arg Val Tyr Tyr Arg Asp Asn Arg Asp Pro Leu Trp Lys Gly Pro
Ala Arg Leu Leu Trp Lys Gly Glu Gly Ala Val Val Ile Gln Asp Asn Ser Glu Ile Lys Val Val Pro
Arg Arg Lys Val Lys Ile Ile Arg Asp Tyr Gly Lys Arg Met Ala Gly Asp Asp Cys Val Ala Gly
Arg Gln Asp Glu Asp Gly Thr Val Leu Ile Gly Pro Thr Pro Val Asn Ile Ile Gly Arg Asn Leu Leu
Thr Gln Leu Gly Cys Thr Leu Asn Phe Pro Ile Ser Pro Ile Asp Thr Val Pro Val Lys Leu Lys Pro
Gly Met Asp Gly Pro Arg Val Lys Gln Trp Pro Leu Thr Glu Glu Lys Ile Lys Ala Leu Ile Glu Ile
Cys Thr Glu Met Glu Lys Glu Gly Lys Ile Ser Arg Ile Gly Pro Glu Asn Pro Tyr Asn Thr Pro Ile
Phe Ala Ile Lys Lys Lys Asp Gly Thr Lys Trp Arg Lys Leu Val Asp Phe Arg Glu Leu Asn Lys
Lys Thr Gln Asp Phe Trp Glu Val Gln Leu Gly Ile Pro His Pro Ser Gly Leu Lys Lys Lys Lys Ser
Val Thr Val Leu Asp Ile Gly Asp Ala Tyr Phe Ser Val Pro Leu Asp Lys Glu Phe Arg Lys Tyr Thr
Ala Phe Thr Val Pro Ser Thr Asn Asn Glu Thr Pro Gly Val Arg Tyr Gln Tyr Asn Val Leu Pro
Met Gly Trp Lys Gly Ser Pro Ala Ile Phe Gln Cys Ser Met Thr Lys Glu Lys Ile Tyr Leu Ala Trp
Val Pro Ala His Lys Gly Ile Gly Gly Asn Glu Gln Ile Asp Lys Leu Val Ser
(SEQ ID NO: 411)
Gln Leu Lys Gly Glu Ala Met His Gly Gln Val Asp Cys Ser Pro Gly
Ile Trp Gln Leu Asp Cys Thr His Leu Cys Ser Ala Thr Glu Lys Leu Trp Val Thr Val Tyr Tyr Gly
Val Pro Val Trp Lys Glu Ala Thr Thr Thr Leu Ala Ser Arg Glu Leu Glu Arg Phe Ala Val Asn
Pro Gly Leu Leu Asn Asn Glu Thr Pro Gly Ile Arg Tyr Gln Tyr Asn Val Leu Pro Gln Gly Trp
Lys Gly Ser Pro Ala Ile Phe Pro Val Gly Glu Ile Tyr Lys Arg Trp Ile Ile Leu Gly Leu Asn Lys Ile
Val Arg Met Tyr Ser Pro Thr Ser Ile Ala Ala Pro Pro Phe Leu Trp Met Gly Tyr Glu Leu His Pro
Asp Lys Trp Thr Val Gln Pro Ile Val Leu Pro Glu Lys Trp Glu Thr Trp Trp Thr Glu Tyr Trp Gln
Ala Thr Trp Ile Pro Glu Trp Glu Phe Val Asn Thr Pro Pro Leu Gln Asp Ser Gly Leu Glu Val Asn
Ile Val Thr Asp Ser Gln Tyr Ala Leu Gly Ile Ile Gln Ala Gln Pro Asp Ser Trp Thr Val Asn Asp
Ile Gln Lys Leu Val Gly Lys Leu Asn Trp Ala Ser Gln Ile Tyr Pro Gly Ile Lys Ala Ala Asp Pro
Leu Trp Lys Gly Pro Ala Lys Leu Leu Trp Lys Gly Glu Gly Ala Val Val Ile Gln Asp Asn Ser Asp
Arg Gln Ala Asn Phe Leu Gly Lys Ile Trp Pro Ser His Lys Gly Arg Pro Leu Trp Lys Gly Pro Ala
Lys Leu Leu Trp Lys Gly Glu Gly Ala Val Val Ile Gln Asp Asn Ser Asp Ile Ala Ala Ser Asn Phe
Thr Ser Thr Thr Val Lys Ala Ala Cys Trp Trp Ala Gly Ile Lys Gln Glu Phe Gly Ile Pro Tyr Val
Leu Pro Glu Lys Asp Ser Trp Thr Val Asn Asp Ile Gln Lys Leu Val Gly Lys Leu Asn Trp Ala
Ser Gln Met Val His Gln Ala Ile Ser Pro Arg Thr Leu Asn Ala Trp Val Lys Val Val Glu Glu Lys
Ala Phe Ser Pro Ile Val Leu Pro Glu Lys Asp Ser Trp Thr Val Asn Asp Ile Gln Lys Leu Val Gly
Lys Leu Asn Trp Ala Ser Gly Thr Gly Pro Cys Thr Asn Val Ser Thr Val Gln Cys Thr His Gly Ile
Arg Pro Val Val Ser Thr Gln Leu Tyr Leu Lys Asp Gln Gln Leu Leu Gly Ile Trp Gly Cys Ser Gly
Lys Leu Ile Cys Thr Thr Ala Val Pro Trp Ala Ala Ala Lys Lys His Gln Lys Glu Pro Pro Phe Leu
Trp Met Gly Tyr Glu Leu His Pro Asp Lys Trp Thr Val Gln Pro Val Tyr Tyr Arg Asp Ser Arg
Asp Pro Leu Trp Lys Gly Pro Ala Lys Leu Leu Trp Lys Gly Glu Gly Ala Val Asn Leu Leu Thr
Gln Ile Gly Cys Thr Leu Asn Phe Pro Ile Ser Pro Ile Glu Thr Val Pro Val Lys Leu Lys Gly Pro
Lys Glu Pro Phe Arg Asp Tyr Val Asp Arg Phe Tyr Lys Thr Leu Arg Ala Glu Gln Ala Ser Gln
Glu Val Leu Gly Pro Ala Ala Thr Leu Glu Glu Met Met Thr Ala Cys Gln Gly Val Gly Gly Pro
Gly His Lys Ala Arg Asp Gln Ser Leu Lys Pro Cys Val Lys Leu Thr Pro Leu Cys Val Thr Leu
Asn Cys Thr Asp Leu Arg Asn Thr Gly Pro Gly Ile Arg Tyr Pro Leu Leu Thr Phe Gly Trp Cys
Phe Lys Leu Pro Val Glu Pro Glu Lys Val Glu Ala Lys Ile Ile Arg Asp Tyr Gly Lys Gln Met Ala
Gly Asp Asp Cys Val Ala Ser Arg Gln Asp Glu Asp Ile Leu Lys Ala Leu Gly Pro Ala Ala Thr
Leu Glu Glu Met Met Thr Ala Cys Gln Gly Val Gly Gly Pro Gly Tyr Lys Ala Ala Val Asp Leu
Ser His Phe Leu Arg Glu Lys Gly Gly Leu Glu Gly Ala Ala Tyr Ala Ala Lys Ala Tyr Asp Thr
Glu Val His Asn Val Trp Ala Thr His Ala Cys Val Pro Thr Asp Pro Asn Pro Gln Glu Ala Ala Ala
Leu Ser Glu Gly Ala Thr Pro Gln Asp Leu Asn Thr Met Leu Asn Thr Val Gly Gly His Gln Ala
Ala Met Gln Gln Val Asp Cys Ser Pro Gly Ile Trp Gln Leu Asp Cys Thr His Leu Glu Gly Lys Ile
Ile Leu Val Ala Val
(SEQ ID NO: 412)
Gly Ser Glu Phe Thr Leu Ile Pro Ile Ala Val Gly Gly Ala Leu Ala
Gly Leu Val Ile Val Leu Ile Ala Tyr Leu Val Gly Arg Lys Arg Ser His Ala Gly Tyr Gln Thr Ile
(SEQ ID NO: 413)
Met Asn Gln Thr Ala Ile Leu Ile Cys Cys Leu Ile Phe Leu Thr Leu
Ser Gly Ile Gln Gly
(SEQ ID NO: 414)
atgggagcta gagctagcgt gctgagcgga ggagaactcg atcgctggga aaagatcaga
ctgagaccag gaggcaagaa gaagtacaga ctgaagcaca tcgtctgggc ttctagagaa ctggaaagat tcgccgtgaa
tccaggactg ctggaaacac tgaagcacat tgtctgggct agcagagaac tggagagatt tgccgtgaat ccaggactgc
tggaaacagc agctatctct cctagaacac tgaacgcttg ggtgaaagtg gtggaggaaa aggcctttag cccagaagtg
atccctatgt ttagcgccct gtcagaagga gctacacctc aggatctgaa caccatgctg aacacagtgg gaggacatca
ggcagctatg cagatgctga aggagacaat taacgaagaa gccgccgagt gggatagact gcatccagtg cacgcaggac
ctattgctcc aggacagatg agagagccta gaggaagcga tatcgcagga acaacatcta cactgcagga gcagatcggt
tggatgacca ataatcctcc tatcccagtg ggcgaaatct ataagcgctg gatcatcctg ggactgaaca agatcgtgag
gatgtacagc cctaccagca tcctggatat cagacaggga cctaaggagc ctttcagaga ttacgtggac aggttctaca
agacactgag agccgaacag gcttctcagg aggtgaagaa ttggatgacc gagacactgc tggtgcagaa cgctaatcca
gattgcaaga caattctgaa agctctggga ccagccgcta cactggaaga gatgatgacc gcttgtcagg gagtgggagg
accaggacat aaagctagag tgctggcaga agccatgtct caggaagaag tgggattccc agtgaaacct caggtgcctc
tgagacctat gacctttaag ggagctctgg acctgtctca cttcctgaga gaaaagggag gactggaagg aacacaggga
tttttcccag atcagaatta cacaccagag ccaggaatca gattccctct gacattcggt tggtgcttca aactggtgcc tctg
(SEQ ID NO: 415)
ggaacagtgc tggtgggacc tactccagtg aatatcatcg gaaggaacct gctgacacag
attggttgta ccctgaactt ccctatctct cctatcgaga cagtgccagt gaaactgaag ccaggaatgg atggacctaa
agtcaagcag tggcctctga cagaagagaa gatcaaagcc ctggtggaga tttgcaccga gatggagaag gagggaaaga
tcagcaagat cggcccagag aatccttaca acaccccagt gttcgccatc aagaagaagg atagcaccaa gtggagaaag
ctggtggatt tcagggagct gaacaagaga acccaggatt tttgggaggt gcagctgggt attccacatc ctgccggact
gaaaaagaag aaaagcgtga cagtgctgga cgtgggagac gcttatttca gcgtgcctct ggataaggac ttcagaaagt
acaccgcctt caccatccct tctatcaaca acgagacccc aggaatcaga taccagtaca acgtgctgcc tcaaggttgg
aaaggatctc cagccatctt tcagagcagc atgacaacag tgaaggcagc ttgttggtgg gcaggaatta agcaggagtt
cggcatccct tacaatcctc agtctcaggg agtggtggaa tctatgaaca aggagctgaa gaagatcatc ggacaggtga
gagatcaggc cgaacatctg aagacagcag tgcaaatggc cgtgttcatc cacaacttca agagaaaggg cggcattgga
ggctattctg ccggagagag aattgtggac atcatcaacg tgtcaacagt ccagtgtaca cacggaatca gaccagtcgt
gtctacacaa ctgctgctga acggatctct ggccgaagag aagagaagag tggtgcagag agagaaaaga gcagtgggaa
tcggagctat gtttctggga tttctgggcg cagcaggatc tacaatggga gcagcttcta tcacactgac agtgcaggct
agacaactgc tgagcggaat tgtgcagcag cagaataacc tgctgagagc tatcgaagct cagcaacatc tgctgcaact
caccgtctgg ggaattaagc aactgcaagc tagagtgctg gcagtggaaa gatacctgaa ggatcagcaa ctgctgggaa
tttggggttg ctcaggcaag ctgatttgca caaccgtggc caaagagatt gtggcttctt gcgacaagtg tcagctgaaa
ggagaagcta tgcacggaca agtggattgt tctccaggaa tttggcagct ggattgtaca cacctggagg gaaagattat
tctggtggca gtgcacgtgg ccagcggata tattgaagcc gaggtgattc cagcagaaac aggacaggaa acagcctatt
ttctcctgaa actggcaggt aggtggccag tgaaaaccct ctgggtgaca gtgtactacg gagtcccagt ctggaaagaa
gcagctttcc ctcagattac tctctggcag agacctctgg tgacaatcaa gatcggcgga cagctgaaag aagctctgct
ggatacagga gcagacgata cagtgctgga agaaatgaac ctgccaggta gatggaagcc taagatgatc ggaggcatcg
gaggattcat caaggtgaga cagtacgacc aagcagcagc agctcataac gtctgggcta cacacgcttg cgtgcctaca
gatcctaatc ctcaggaagc catcaccaag atccagaatt tcagggtgta ctacagggac agcagagatc ctctctggaa
aggaccagct aaactgctgt ggaaaggaga aggagcagtg gtgatccagg ataacagcga catcaaggtg gtgcctagaa
gaaaggccaa gatcatcagg gactacggaa agcaaatggc aggagacgat tgcgtggctt ctagacagga cgaggatccc
aagttcaagc tgcctattca gaaggagact tgggagactt ggtggacaga gtattggcaa gcaacttgga tccccgagtg
acgtggacgg agcagctaat agagagacaa aagccgccaa ggagaaagtg tatctggctt gggtgccagc tcataaagga
atcggaggaa acgagcaggt ggataaactg gtgtcttggg gctttaccac accagataag aagcaccaga aggagccacc
atttctctgg atgggatacg aactgcaccc agataagtgg acagtccagc ctattgtgct gccagaaaag gactcttgga
cagtgaacga catccagaaa ctggtgggaa agctgaattg ggcctctcag atctacccag gcatcaaggt gatcgtgatc
taccagtaca tggacgatct gtacgtggga tcagatctgg agatcggaca gcacagaatg agggacaatt ggagaagcga
gctgtacaag tacaaggtgg tg
(SEQ ID NO: 416)
taccagtata acgtgctgcc tcagggagct tctagagaac tggagagatt cgcagtgaac ccaggactcc tctggattat
cctgggactg aacaagatcg tgaggatgta ctctcctacc tctattgccg ctagaacact gaacgcttgg gtgaaggtct
tcctctggat gggatacgaa ctgcatctga cctttggttg gtgctttaag ctccctctct ggaaaggacc agctaagctg
gtgacagtgt attacggagt gccagtggca gctctcctct ggaaaggaga aggagcagtg gcagcagcta aactggtggg
aaagctgaat tgggccaaac tcctctggaa gggagaagga gccaccctga attttcctat cagccctatt tggcaggcta
cttggattcc agagtggaaa gcagcttgtt ggtgggcagg aatcagacag gccaacttcc tgggcaagat ttggccttct
cacaaaggaa gaaacgtctg ggctacacac gcttgcgtgg cagcagaaat gatgacagct tgtcagggag tgtctacagt
ccagtgtaca cacggaatcg cagctaaaca gatggcagga gacgattgcg tggcagcttg gcagctggat tgtacacacc
tggagtacaa ggcagcagtg gatctgtctc actttctgag agaaaaagga ggactggaag gagcagctta ctacatggac
gatctgtacg tgggatcagg acaggtggat tgttcaccag gaatcgctac actggaggaa atgatgaccg cagaactgca
tccagataag tggaccgtct ggacagtgaa cgatatccag aagctgggca tttggggttg tagcggaaaa ctgaccgtga
acgatatcca gaagctggtg atcgtgaccg attctcagta cgctctgtac gtggacagat tctacaagac cctgtacgtg
gacaggttct acaagacact gagagccgaa caggcttctc aggaagtgga tctgaacacc atgctgaaca ccgtgaaact
gacacctctc tgcgtgacac tgtatcagta catggacgac ctgtacgtgg tgatctacca gtacatggac gatctctgga
tcatcctggg actgaacaag atcg
(SEQ ID NO: 417)
agaacactga acgcttgggt gaaggtgaga gagaagagag acctgaacac catgctgaac
ccgtgagag aaaagaggtg gatcatcctg ggactgaaca agatcaggga gaagaggtac gtggacaggt tctacaagac
actgagagag aagagagcca cactggaaga gatgatgacc gctagagaga agagagagat gatgaccgct tgtcagggag
tgagagagaa gagaaccctg aacttcccca tctctcctat cagggagaag aggtaccagt acaacgtgct gcctcaggga
agagaaaaga gagtgatcta ccagtacatg gacgacctga gagagaagag gtaccagtac atggacgatc tgtacgtgag
ggagaagaga tacatggacg acctgtacgt gggatcaaga gagaagagat tcctctggat gggctacgag ctgcatagag
agaagagaga gctgcaccca gataagtgga cagtgagaga aaagcgctgg acagtgaacg acatccagaa gctgagagag
aagaggacag tgaacgacat ccagaagctg gtgagagaga agaggaagct ggtgggaaaa ctgaattggg ctagggaaaa
aaggtggcag gctacttgga ttccagagtg gagagagaag aggatcgtga cagatagcca gtacgctctg agagagaaaa
gaggacaggt ggattgctct ccaggaatca gagagaagag atggcagctg gattgtacac acctggagag agagaagagg
aaagcagctt gttggtgggc aggaattcgg gaaaaaagac ctctctggaa aggaccagcc aagctgagag agaagagaaa
actcctctgg aagggcgaag gagctagaga aaagagactc ctctggaaag gagaaggcgc agtgagagag aagagaaaac
agatggccgg agacgattgc gtgagagaaa agagagtgac cgtgtattac ggagtgccag
tgggctacac acgcttgcgt gagagagaag agaaagctga cacctctgtg cgtgacactg agagaaaaga gaagcaccgt
gcagtgtaca cacggaatta gggagaagag aggcatttgg ggttgttcag gaaagctgag agagaagagg ctgacattcg
gttggtgttt caagctgagg gagaagagag cctctagaga actggagaga ttcgcagtga atccaggact gctgagagaa
aagcgctgga ttatcctggg actgaacaag atcgtgagga tgtacagccc tacaagcatc agagagaaga ggtacgtgga
cagattctac aagaccctga gagccgaaca ggcatctcag gaagtgagag agaagagaag gcaggctaac ttcctgggaa
agatttggcc tagccacaag ggaagaagag agaagagata caaggccgca gtggatctgt ctcactttct gagagagaaa
ggaggactgg aaggagga
(SEQ ID NO: 418)
atggctccta gaagcgctag aagacctctg ctgctgctgc tgctgctgct gctgctggga ctgatgcatt gcgcttcagc
agctatgttc atggtgaaga acggcaacgg aacagcttgt atcatggcca atttcagcgc cgcttttagc gtgaattacg
acaccaagag cggacctaag aacatgacac tggatctgcc ttcagacgct acagtggtgc tgaatagaag ctcttgcgga
aaggagaata cctccgatcc ttctctggtg atcgcttttg gcagaggaca cacactgaca ctgaacttca ccagaaacgc
caccagatac tcagtgcagc tgatgagctt cgtgtacaac ctgagcgata cccatctgtt tcctaacgct agcagcaagg
agatcaagac agtggagtct atcaccgaca tcagagccga tatcgacaag aaataccgct gcgtgtcagg aacacaggtg
cacatgaaca acgtgacagt gacactgcac gacgccacaa ttcaggccta tctgagcaat agcagcttta gcagaggcga
aactaggtgt gagcaggata gaccttctcc tacaacagct cctccagctc ctccttctcc ttctccttct ccagtgccta
aatctcctag cgtggataag tacaacgtga gcggaacaaa cggcacttgt ctgctggctt ctatgggact gcagctgaat
ctgacatacg agaggaagga caacaccaca gtgacaagac tgctgaacat caaccccaac aaaacaagcg ctagcggatc
ttgcggagct catctggtga cactggaact gcattcagag ggaacaacag tgctgctgtt tcagttcgga atgaacgcct
ctagcagcag attcttcctg cagggtattc agctgaatac actgctgcca gatgctagag atccagcctt taaagccgct aatggatctc
tgagagctct gcaggctaca gtgggaaata gctacaagtg caacgccgaa gaacacgtga gagtgacaaa agccttcagc
gtgaacatct ttaaggtctg ggtgcaggca tttaaagtgg agggaggcca gtttggaagc gtcgaagagt gtctgctgga
cgaaaatagc ctggaagaca tcagaacact gaacgcttgg gtgaaggtga gagagaagag agacctgaac accatgctga
acaccgtgag agaaaagagg tggatcatcc tgggactgaa caagatcagg gagaagaggt acgtggacag gttctacaag
acactgagag agaagagagc cacactggaa gagatgatga ccgctagaga gaagagagag atgatgaccg cttgtcaggg
agtgagagag aagagaaccc tgaacttccc catctctcct atcagggaga agaggtacca gtacaacgtg ctgcctcagg
gaagagaaaa gagagtgatc taccagtaca tggacgacct gagagagaag aggtaccagt acatggacga tctgtacgtg
agggagaaga gatacatgga cgacctgtac gtgggatcaa gagagaagag attcctctgg atgggctacg agctgcatag
agagaagaga gagctgcacc cagataagtg gacagtgaga gaaaagcgct ggacagtgaa cgacatccag aagctgagag
agaagaggac agtgaacgac atccagaagc tggtgagaga gaagaggaag ctggtgggaa aactgaattg ggctagggaa
aaaaggtggc aggctacttg gattccagag tggagagaga agaggatcgt gacagatagc cagtacgctc tgagagagaa
aagaggacag gtggattgct ctccaggaat cagagagaag agatggcagc tggattgtac acacctggag agagagaaga
ggaaagcagc ttgttggtgg gcaggaattc gggaaaaaag acctctctgg aaaggaccag ccaagctgag agagaagaga
aaactcctct ggaagggcga aggagctaga gaaaagagac tcctctggaa aggagaaggc gcagtgagag agaagagaaa
acagatggcc ggagacgatt gcgtgagaga aaagagagtg accgtgtatt acggagtgcc agtgagagaa aagagaaacg
tctgggctac acacgcttgc gtgagagaga agagaaagct gacacctctg tgcgtgacac tgagagaaaa gagaagcacc
gtgcagtgta cacacggaat tagggagaag agaggcattt ggggttgttc aggaaagctg agagagaaga ggctgacatt
cggttggtgt ttcaagctga gggagaagag agcctctaga gaactggaga gattcgcagt gaatccagga ctgctgagag
aaaagcgctg gattatcctg ggactgaaca agatcgtgag gatgtacagc cctacaagca tcagagagaa gaggtacgtg
gacagattct acaagaccct gagagccgaa caggcatctc aggaagtgag agagaagaga aggcaggcta acttcctggg
aaagatttgg cctagccaca agggaagaag agagaagaga tacaaggccg cagtggatct gtctcacttt ctgagagaga
aaggaggact ggaaggagga agcgagttta ccctgattcc aattgccgtg ggaggagctc tggcaggact ggtgattgtg
ggaaagcagc ttgttggtgg gcaggaattc gggaaaaaag acctctctgg aaaggaccag ccaagctgag agagaagaga
aaactcctct ggaagggcga aggagctaga gaaaagagac tcctctggaa aggagaaggc gcagtgagag agaagagaaa
acagatggcc ggagacgatt gcgtgagaga aaagagagtg accgtgtatt acggagtgcc agtgagagaa aagagaaacg
tctgggctac acacgcttgc gtgagagaga agagaaagct gacacctctg tgcgtgacac tgagagaaaa gagaagcacc
gtgcagtgta cacacggaat tagggagaag agaggcattt ggggttgttc aggaaagctg agagagaaga ggctgacatt
cggttggtgt ttcaagctga gggagaagag agcctctaga gaactggaga gattcgcagt gaatccagga ctgctgagag
aaaagcgctg gattatcctg ggactgaaca agatcgtgag gatgtacagc cctacaagca tcagagagaa gaggtacgtg
gacagattct acaagaccct gagagccgaa caggcatctc aggaagtgag agagaagaga aggcaggcta acttcctggg
aaagatttgg cctagccaca agggaagaag agagaagaga atcaaggccg cagtggatct gtctcacttt atgagagaga
aaggaggact ggaaggagga agcgagttta ccctgattcc aattgccgtg ggaggagctc tggcaggact ggtgattgtg
ctgatcgcat acctggtggg aagaaagaga tctcacgccg gatatcagac catc
Gly Gly Ser
Gly Ser Gly
(SEQ ID NO: 421)
Gly Gly Gly Ser
(SEQ ID NO: 422)
Ile Cys Gly His Lys Ala Ile Gly Thr Val Leu Val Gly Pro Thr Pro
Val Asn Ile Ile Gly Arg Asn Leu Leu Thr Gln Leu Gly Cys Thr Leu Asn Phe Pro Ile Ser Pro Ile
Glu Thr Val Pro Val Lys Leu Lys Pro Gly Met Asp Gly Pro Lys Val Lys Gln Trp Pro Leu Thr
Glu Glu Lys Ile Lys Ala Leu Asp Pro Leu Trp Lys Gly Pro Ala Lys Leu Leu Trp Lys Gly Glu
Gly Ala Val Val Ile Gln Asp Asn Ser Asp Ile Lys Val Val Pro Arg Arg Lys Ala Lys Ile Ile Arg
Asp Tyr Gly Lys Gln Met Ala Ala Ala Tyr Ser Asp Ile Ala Gly Thr Thr Ser Thr Leu Gln Glu Gln
Ile Thr Trp Met Thr Asn Asn Pro Pro Ile Pro Val Gly Glu Ile Tyr Lys Arg Trp Ile Ile Leu Gly Leu
Asn Lys Ile Val Arg Met Tyr Ser Pro Val Ser Ile Leu Asp Ile Arg Gln Gly Pro Lys Glu Pro Phe
Arg Asp Tyr Val Asp Arg Phe Tyr Lys Thr Leu Arg Ala Glu Gln Ala Ser Gln Glu Val Lys Asn
Trp Met Thr Glu Thr Leu Leu Val Gln Asn Ala Asn Pro Asp Cys Lys Thr Ile Leu Lys Ala Leu
Gly Pro Ala Ala Thr Leu Glu Glu Met Met Thr Ala Cys Gln Gly Val Gly Gly Pro Gly His Lys
Ala Arg Val Leu Ala Glu Ala Met Ser Gln Val Thr Asn Ser Ala Thr Leu Asn Lys Arg Thr Gln
Asp Phe Trp Glu Val Gln Leu Gly Ile Pro His Pro Ala Gly Leu Lys Lys Lys Lys Ser Asn Phe Thr
Ser Thr Thr Val Lys Ala Ala Cys Trp Trp Ala Gly Ile Lys Gln Glu Phe Gly Ile Pro Tyr Asn Pro
Gln Ser Ala Tyr Phe Ser Val Pro Leu Asp Lys Glu Phe Arg Lys Tyr Thr Ala Phe Thr Ile Pro Ser
Ile Asn Asn Glu Asp Thr Val Leu Glu Glu Met Asn Leu Pro Gly Lys Trp Lys Pro Lys Met Ile
Gly Gly Ile Gly Gly Phe Ile Lys Val Arg Gln Tyr Asp Gln Ile Ser Lys Ile Gly Pro Glu Asn Pro
Tyr Asn Thr Pro Ile Phe Ala Ile Lys Lys Lys Asp Ser Thr Lys Trp Ala Ala Gly Lys Lys Lys Tyr
Arg Leu Lys His Leu Val Trp Val Ser Arg Glu Leu Glu Arg Phe Ala Val Asn Pro Gly Gly Lys
Lys Lys Tyr Arg Leu Lys His Leu Val Trp Ala Ser Arg Glu Leu Glu Arg Phe Ala Val Asn Pro
Gly Ala Glu His Leu Lys Thr Ala Val Gln Met Ala Val Phe Ile His Asn Phe Lys Arg Lys Gly Gly
Ile Gly Gly Ala Ala Gly Gln Met Val His Gln Ala Ile Ser Pro Arg Thr Leu Asn Ala Trp Val Lys
Val Val Glu Glu Lys Ala Phe Ser Pro Glu Val Ile Pro Met Phe Ser Ala Leu Ala Glu Gly Ala Thr
Pro Gln Asp Leu Asn Thr Met Leu Asn Thr Val Gly Gly His Gln Ala Arg Trp Ile Ile Leu Gly Leu
Asn Lys Thr Val Arg Met Tyr Ser Pro Val Ser Ile Leu Asp Ile Arg Gln Gly Pro Lys Glu Pro Phe
Arg Asp Tyr Val Asp Arg Phe Tyr Lys Thr Leu Arg Ala Glu Gln Ala Ser Gln Glu Val Lys Asn
Trp Met Thr Glu Thr Leu Leu Val Gln Asn Ala Asn Pro Asp Cys Lys Thr Ile Leu Lys Ala Leu
Gly Pro Ala Ala Thr Leu Glu Glu Met Met Thr Ala Cys Gln Gly Val Gly Gly Pro Gly His Lys
Ala Arg Val Leu Ala Glu Ala Met Ser Gln Val Thr Asn Ser Ala Thr Gln Leu Lys Gly Glu Ala
Met His Gly Gln Val Asp Cys Ser Pro Gly Ile Trp Gln Leu Asp Cys Thr His Leu Glu Gly Lys Val
Ile Leu Val Ala Val His Val Ala Ser Gly Tyr Ile Glu Ala Glu Val Ile Pro Ala Glu Thr Gly Gln
Glu Thr Ala Tyr Phe Leu Leu Lys Leu Ala Gly Arg
(SEQ ID NO: 423)
Ser Asn Phe Thr Ser Thr Thr Val Lys Val Ala Cys Trp Trp Ala Gly
Ile Lys Gln Glu Phe Gly Ile Pro Tyr Ala Ala Ser Asn Phe Thr Ser Thr Thr Val Lys Ala Ala Cys
Trp Trp Ala Gly Val Lys Gln Glu Phe Gly Ile Pro Tyr Ala Ala Ser Asn Phe Thr Ser Thr Thr Val
Lys Ala Ala Cys Trp Trp Ala Gly Ile Lys Gln Glu Phe Gly Ile Pro Tyr Pro Leu Arg Pro Met Thr
Tyr Lys Ala Ala Val Asp Leu Ser His Phe Leu Lys Glu Lys Gly Gly Leu Glu Gly Pro Leu Arg
Pro Met Thr Tyr Lys Ala Ala Val Asp Leu Ser Phe Phe Leu Lys Glu Lys Gly Gly Leu Glu Gly
Pro Leu Arg Pro Met Thr Tyr Lys Gly Ala Phe Asp Leu Ser Phe Phe Leu Lys Glu Lys Gly Gly
Leu Glu Gly Pro Leu Arg Pro Met Thr Tyr Lys Ala Ala Phe Asp Leu Ser Phe Phe Leu Lys Glu
Lys Gly Gly Leu Glu Gly Pro Leu Arg Pro Met Thr Tyr Lys Ala Ala Phe Asp Leu Ser His Phe
Leu Lys Glu Lys Gly Gly Leu Glu Gly Pro Leu Arg Pro Met Thr Tyr Lys Gly Ala Leu Asp Leu
Ser His Phe Leu Lys Glu Lys Gly Gly Leu Glu Gly Gln Leu Lys Gly Glu Ala Met His Gly Gln
Val Asp Cys Ser Pro Gly Ile Trp Gln Leu Asp Cys Thr His Leu Glu Glu Lys Ile Ile Leu Val Ala
Val His Val Ala Ser Gly Tyr Ile Glu Ala Glu Val Ile Pro Ala Glu Thr Gly Gln Glu Thr Ala Tyr
Met Val His Gln Ala Ile Ser Pro Arg Thr Leu Asn Ala Trp Val Lys Val Val Glu Glu Lys Ala Phe
Ser Pro Leu Asp Cys Thr His Leu Glu Gly Lys Val Ile Leu Val Ala Val His Val Ala Ser Gly Tyr
Ile Glu Ala Glu Ile Cys Gly His Lys Ala Ile Gly Thr Val Leu Val Gly Pro Thr Pro Val Asn Ile Ile
Gly Arg Asn Leu Leu Thr Gln Ile Gly Cys Thr Leu Asn Phe Pro Ile Ser Pro Ile Glu Thr Val Pro
Val Lys Leu Lys Asp Pro Leu Trp Lys Gly Pro Ala Lys Leu Leu Trp Lys Gly Glu Gly Ala Val
Val Ile Gln Asp Asn Ser Asp Ile Asp Pro Leu Trp Lys Gly Pro Ala Lys Leu Leu Trp Lys Gly Glu
Gly Val Val Val Ile Gln Asp Asn Ser Asp Ile Met Val His Gln Ala Ile Ser Pro Arg Thr Leu Asn
Ala Leu Val Lys Val Val Glu Glu Lys Ala Phe Ser Pro Ile Cys Gly His Lys Ala Ile Gly Thr Val
Leu Val Gly Ser Thr Pro Val Asn Ile Ile Gly Arg Asn Leu Leu
(SEQ ID NO: 424)
Ile Arg Thr Leu Asn Ala Trp Val Lys Val Arg Glu Lys Arg Asp Leu
Asn Thr Met Leu Asn Thr Val Arg Glu Lys Arg Trp Ile Ile Leu Gly Leu Asn Lys Ile Arg Glu Lys
Arg Tyr Val Asp Arg Phe Tyr Lys Thr Leu Arg Glu Lys Arg Ala Thr Leu Glu Glu Met Met Thr
Ala Arg Glu Lys Arg Glu Met Met Thr Ala Cys Gln Gly Val Arg Glu Lys Arg Thr Leu Asn Phe
Pro Ile Ser Pro Ile Arg Glu Lys Arg Tyr Gln Tyr Asn Val Leu Pro Gln Gly Arg Glu Lys Arg Val
Ile Tyr Gln Tyr Met Asp Asp Leu Arg Glu Lys Arg Tyr Gln Tyr Met Asp Asp Leu Tyr Val Arg
Glu Lys Arg Tyr Met Asp Asp Leu Tyr Val Gly Ser Arg Glu Lys Arg Phe Leu Trp Met Gly Tyr
Glu Leu His Arg Glu Lys Arg Glu Leu His Pro Asp Lys Trp Thr Val Arg Glu Lys Arg Trp Thr
Val Asn Asp Ile Gln Lys Leu Arg Glu Lys Arg Thr Val Asn Asp Ile Gln Lys Leu Val Arg Glu
Lys Arg Lys Leu Val Gly Lys Leu Asn Trp Ala Arg Glu Lys Arg Trp Gln Ala Thr Trp Ile Pro
Glu Trp Arg Glu Lys Arg Ile Val Thr Asp Ser Gln Tyr Ala Leu Arg Glu Lys Arg Gly Gln Val
Asp Cys Ser Pro Gly Ile Arg Glu Lys Arg Trp Gln Leu Asp Cys Thr His Leu Glu Arg Glu Lys
Arg Lys Ala Ala Cys Trp Trp Ala Gly Ile Arg Glu Lys Arg Pro Leu Trp Lys Gly Pro Ala Lys Leu
Arg Glu Lys Arg Lys Leu Leu Trp Lys Gly Glu Gly Ala Arg Glu Lys Arg Leu Leu Trp Lys Gly
Glu Gly Ala Val Arg Glu Lys Arg Lys Gln Met Ala Gly Asp Asp Cys Val Arg Glu Lys Arg Val
Thr Val Tyr Tyr Gly Val Pro Val Arg Glu Lys Arg Asn Val Trp Ala Thr His Ala Cys Val Arg
Glu Lys Arg Lys Leu Thr Pro Leu Cys Val Thr Leu Arg Glu Lys Arg Ser Thr Val Gln Cys Thr
His Gly Ile Arg Glu Lys Arg Gly Ile Trp Gly Cys Ser Gly Lys Leu Arg Glu Lys Arg Leu Thr Phe
Gly Trp Cys Phe Lys Leu Arg Glu Lys Arg Ala Ser Arg Glu Leu Glu Arg Phe Ala Val Asn Pro
Gly Leu Leu Arg Glu Lys Arg Trp Ile Ile Leu Gly Leu Asn Lys Ile Val Arg Met Tyr Ser Pro Thr
Ser Ile Arg Glu Lys Arg Tyr Val Asp Arg Phe Tyr Lys Thr Leu Arg Ala Glu Gln Ala Ser Gln Glu
Val Arg Glu Lys Arg Arg Gln Ala Asn Phe Leu Gly Lys Ile Trp Pro Ser His Lys Gly Arg Arg
Glu Lys Arg Tyr Lys Ala Ala Val Asp Leu Ser His Phe Leu Arg Glu Lys Gly Gly Leu Glu Gly
(SEQ ID NO: 425)
Ala Val Lys Ala Ala Cys Trp Trp Ala Gly Val Lys Gln Glu Phe Gly
Ile Pro Tyr Asn Thr Gln Ser Gln Gly Val Val Glu Ser Met Asn Asn Glu Leu Lys Lys Ile Ile Gly
Gln Ile Arg Asp Gln Ala Glu His Leu Lys Thr Ala Val Gln Met Ala Val Leu Ile His Asn Phe Lys
Arg Lys Gly Gly Ile Gly Glu Tyr Ser Ala Gly Glu Arg Ile Ile Asp Ile Ile
(SEQ ID NO: 426)
Ala Val Lys Ala Ala Cys Trp Trp Ala Gly Val Lys Gln Glu Phe Gly
Ile Pro Tyr Asn Thr Gln Ser Gln Gly Val Val Glu Ser Met Asn Asn Glu Leu Lys Lys Ile Ile Gly
Gln Ile Arg Asp Gln Ala Glu His Leu Lys Thr Ala Val Gln Met Ala Val Leu Ile His Asn Phe Lys
Arg Lys Gly Gly Ile Gly Glu Tyr Ser Ala Gly Glu Arg Ile Ile Asp Ile Ile Ala
(SEQ ID NO: 427)
Val Ala Lys Glu Ile Val Ala Cys Cys Asp Lys Cys Gln Leu Lys Gly
Glu Ala Ile His Gly Gln Val Asp Cys Ser Pro Gly Val Trp Gln Leu Asp Cys Thr His Leu Glu Gly
Lys Val Ile Leu Val Ala Val His Val Ala Ser Gly Tyr Ile Glu Ala Glu Ile Ile Pro Thr Glu Thr Gly
Gln Glu Thr Ala Tyr Phe Ile Leu Lys Leu Ala Gly Arg Trp Pro Val Thr Thr
(SEQ ID NO: 428)
Leu Lys His Ile Val Trp Ala Ser Arg Glu Leu Glu Arg Phe Ala Val
Asn Pro Gly Leu Leu Glu Thr Val Ser Gln Asn Tyr Pro Ile Val Gln Asn Ile Ser Pro Arg Thr Leu
Asn Ala Trp Val Lys Val Val Glu Glu Lys Ala Phe Ser Pro Glu Val Ile Pro Met Phe Ser Ala Leu
Ser Glu Gly Ala Thr Pro Gln Asp Leu Asn Thr Met Leu Asn Thr Val Gly Gly His Gln Ala Ala
Met Gln Met Leu Lys Glu Thr Ile Asn Glu Glu Ala Ala Glu Trp Asp Arg Leu His Pro Val His
Ala Gly Pro Ile Ala Pro Gly Gln Met Arg Glu Pro Arg Gly Ser Asp Ile Ala Gly Thr Thr Ser Thr
Leu Gln Glu Gln Ile Gly Trp Met Thr Asn Asn Pro Pro Ile Pro Val Gly Glu Ile Tyr Lys Arg Trp
Ile Ile Leu Gly Leu Asn Lys Ile Val Arg Met Tyr Ser Pro Thr Ser Ile Leu Asp Ile Arg Gln Gly
Pro Lys Glu Pro Phe Arg Asp Tyr Val Asp Arg Phe Tyr Lys Thr Leu Arg Ala Glu Gln Ala Ser
Gln Glu Val Lys Asn Trp Met Thr Glu Thr Leu Leu Val Gln Asn Ala Asn Pro Asp Cys Lys Thr
Ile Leu Lys Ala Leu Gly Pro Ala Ala Thr Leu Glu Glu Met Met Thr Ala Cys Gln Gly Val Gly
Gly Pro Gly His Lys Ala Arg Val Leu Ala Glu Ala Met Ser Gln
(SEQ ID NO: 429)
Leu Pro Gly Arg Trp Lys Pro Lys Met Ile Gly Gly Ile Gly Gly Phe
Ile Lys Val Arg Gln Tyr Asp Gln Gly Thr Val Leu Val Gly Pro Thr Pro Val Asn Ile Ile Gly Arg
Asn Leu Leu Thr Gln Ile Gly Cys Thr Leu Asn Phe Pro Ile Ser Pro Ile Glu Thr Val Pro Val Lys
Leu Lys Pro Gly Met Asp Gly Pro Lys Val Lys Gln Trp Pro Leu Thr Glu Glu Lys Ile Lys Ala Leu
Val Glu Ile Cys Thr Glu Met Glu Lys Glu Gly Lys Ile Ser Lys Ile Gly Pro Glu Asn Pro Tyr Asn
Thr Pro Val Phe Ala Ile Lys Lys Lys Asp Ser Thr Lys Trp Arg Lys Leu Val Asp Phe Arg Glu Leu
Asn Lys Arg Thr Gln Asp Phe Trp Glu Val Gln Leu Gly Ile Pro His Pro Ala Gly Leu Lys Lys Lys
Lys Ser Val Thr Val Leu Asp Val Gly Asp Ala Tyr Phe Ser Val Pro Leu Asp Lys Asp Phe Arg
Lys Tyr Thr Ala Phe Thr Ile Pro Ser Trp Gly Phe Thr Thr Pro Asp Lys Lys His Gln Lys Glu Pro
Pro Phe Leu Trp Met Gly Tyr Glu Leu His Pro Asp Lys Trp Thr Val Gln Pro Ile
(SEQ ID NO: 430)
Met Gly Ala Arg Ala Ser Val Leu Ser Gly Gly Glu Leu Asp Arg Trp
Glu Lys Ile Arg Leu Arg Pro Gly Gly Lys Lys Lys Tyr Arg Leu Lys His Ile Val Trp Ala Ser Arg
Glu Leu Glu Arg Phe Ala Val Asn Pro Gly Leu Leu Glu Thr Leu Lys His Ile Val Trp Ala Ser Arg
Glu Leu Glu Arg Phe Ala Val Asn Pro Gly Leu Leu Glu Thr Ala Ala Ile Ser Pro Arg Thr Leu Asn
Ala Trp Val Lys Val Val Glu Glu Lys Ala Phe Ser Pro Glu Val Ile Pro Met Phe Ser Ala Leu Ser
Glu Gly Ala Thr Pro Gln Asp Leu Asn Thr Met Leu Asn Thr Val Gly Gly His Gln Ala Ala Met
Gln Met Leu Lys Glu Thr Ile Asn Glu Glu Ala Ala Glu Trp Asp Arg Leu His Pro Val His Ala Gly
Pro Ile Ala Pro Gly Gln Met Arg Glu Pro Arg Gly Ser Asp Ile Ala Gly Thr Thr Ser Thr Leu Gln
Glu Gln Ile Gly Trp Met Thr Asn Asn Pro Pro Ile Pro Val Gly Glu Ile Tyr Lys Arg Trp Ile Ile Leu
Gly Leu Asn Lys Ile Val Arg Met Tyr Ser Pro Thr Ser Ile Leu Asp Ile Arg Gln Gly Pro Lys Glu
Pro Phe Arg Asp Tyr Val Asp Arg Phe Tyr Lys Thr Leu Arg Ala Glu Gln Ala Ser Gln Glu Val
Lys Asn Trp Met Thr Glu Thr Leu Leu Val Gln Asn Ala Asn Pro Asp Cys Lys Thr Ile Leu Lys
Ala Leu Gly Pro Ala Ala Thr Leu Glu Glu Met Met Thr Ala Cys Gln Gly Val Gly Gly Pro Gly
His Lys Ala Arg Val Leu Ala Glu Ala Met Ser Gln Glu Glu Val Gly Phe Pro Val Lys Pro Gln Val
Pro Leu Arg Pro Met Thr Phe Lys Gly Ala Leu Asp Leu Ser His Phe Leu Arg Glu Lys Gly Gly
Leu Glu Gly Thr Gln Gly Phe Phe Pro Asp Gln Asn Tyr Thr Pro Glu Pro Gly Ile Arg Phe Pro Leu
Thr Phe Gly Trp Cys Phe Lys Leu Val Pro Leu
(SEQ ID NO: 431)
Tyr Gln Tyr Asn Val Leu Pro Gln Gly Ala Ser Arg Glu Leu Glu Arg
Phe Ala Val Asn Pro Gly Leu Leu Trp Ile Ile Leu Gly Leu Asn Lys Ile Val Arg Met Tyr Ser Pro
Thr Ser Ile Ala Ala Arg Thr Leu Asn Ala Trp Val Lys Val Phe Leu Trp Met Gly Tyr Glu Leu His
Leu Thr Phe Gly Trp Cys Phe Lys Leu Pro Leu Trp Lys Gly Pro Ala Lys Leu Val Thr Val Tyr
Tyr Gly Val Pro Val Ala Ala Leu Leu Trp Lys Gly Glu Gly Ala Val Ala Ala Ala Lys Leu Val Gly
Lys Leu Asn Trp Ala Lys Leu Leu Trp Lys Gly Glu Gly Ala Thr Leu Asn Phe Pro Ile Ser Pro Ile
Trp Gln Ala Thr Trp Ile Pro Glu Trp Lys Ala Ala Cys Trp Trp Ala Gly Ile Arg Gln Ala Asn Phe
Leu Gly Lys Ile Trp Pro Ser His Lys Gly Arg Asn Val Trp Ala Thr His Ala Cys Val Ala Ala Glu
Met Met Thr Ala Cys Gln Gly Val Ser Thr Val Gln Cys Thr His Gly Ile Ala Ala Lys Gln Met Ala
Gly Asp Asp Cys Val Ala Ala Trp Gln Leu Asp Cys Thr His Leu Glu Tyr Lys Ala Ala Val Asp
Leu Ser His Phe Leu Arg Glu Lys Gly Gly Leu Glu Gly Ala Ala Tyr Tyr Met Asp Asp Leu Tyr
Val Gly Ser Gly Gln Val Asp Cys Ser Pro Gly Ile Ala Thr Leu Glu Glu Met Met Thr Ala Glu Leu
His Pro Asp Lys Trp Thr Val Trp Thr Val Asn Asp Ile Gln Lys Leu Gly Ile Trp Gly Cys Ser Gly
Lys Leu Thr Val Asn Asp Ile Gln Lys Leu Val Ile Val Thr Asp Ser Gln Tyr Ala Leu Tyr Val Asp
Arg Phe Tyr Lys Thr Leu Tyr Val Asp Arg Phe Tyr Lys Thr Leu Arg Ala Glu Gln Ala Ser Gln
Glu Val Asp Leu Asn Thr Met Leu Asn Thr Val Lys Leu Thr Pro Leu Cys Val Thr Leu Tyr Gln
Tyr Met Asp Asp Leu Tyr Val Val Ile Tyr Gln Tyr Met Asp Asp Leu Trp Ile Ile Leu Gly Leu Asn
Lys Ile
(SEQ ID NO: 432)
Met Trp Leu Gln Ser Leu Leu Leu Leu Gly Thr Val Ala Cys Ser Ile
Ser Val Tyr Gln Tyr Asn Val Leu Pro Gln Gly Ala Ser Arg Glu Leu Glu Arg Phe Ala Val Asn
Pro Gly Leu Leu Trp Ile Ile Leu Gly Leu Asn Lys Ile Val Arg Met Tyr Ser Pro Thr Ser Ile Ala
Ala Arg Thr Leu Asn Ala Trp Val Lys Val Phe Leu Trp Met Gly Tyr Glu Leu His Leu Thr Phe
Gly Trp Cys Phe Lys Leu Pro Leu Trp Lys Gly Pro Ala Lys Leu Val Thr Val Tyr Tyr Gly Val
Pro Val Ala Ala Leu Leu Trp Lys Gly Glu Gly Ala Val Ala Ala Ala Lys Leu Val Gly Lys Leu
Asn Trp Ala Lys Leu Leu Trp Lys Gly Glu Gly Ala Thr Leu Asn Phe Pro Ile Ser Pro Ile Trp Gln
Ala Thr Trp Ile Pro Glu Trp Lys Ala Ala Cys Trp Trp Ala Gly Ile Arg Gln Ala Asn Phe Leu Gly
Lys Ile Trp Pro Ser His Lys Gly Arg Asn Val Trp Ala Thr His Ala Cys Val Ala Ala Glu Met Met
Thr Ala Cys Gln Gly Val Ser Thr Val Gln Cys Thr His Gly Ile Ala Ala Lys Gln Met Ala Gly Asp
Asp Cys Val Ala Ala Trp Gln Leu Asp Cys Thr His Leu Glu Tyr Lys Ala Ala Val Asp Leu Ser
His Phe Leu Arg Glu Lys Gly Gly Leu Glu Gly Ala Ala Tyr Tyr Met Asp Asp Leu Tyr Val Gly
Ser Gly Gln Val Asp Cys Ser Pro Gly Ile Ala Thr Leu Glu Glu Met Met Thr Ala Glu Leu His Pro
Asp Lys Trp Thr Val Trp Thr Val Asn Asp Ile Gln Lys Leu Gly Ile Trp Gly Cys Ser Gly Lys Leu
Thr Val Asn Asp Ile Gln Lys Leu Val Ile Val Thr Asp Ser Gln Tyr Ala Leu Tyr Val Asp Arg Phe
Tyr Lys Thr Leu Tyr Val Asp Arg Phe Tyr Lys Thr Leu Arg Ala Glu Gln Ala Ser Gln Glu Val
Asp Leu Asn Thr Met Leu Asn Thr Val Lys Leu Thr Pro Leu Cys Val Thr Leu Tyr Gln Tyr Met
Asp Asp Leu Tyr Val Val Ile Tyr Gln Tyr Met Asp Asp Leu Trp Ile Ile Leu Gly Leu Asn Lys Ile
(SEQ ID NO: 433)
Met Asp Ala Met Lys Arg Gly Leu Cys Cys Val Leu Leu Leu Cys
Gly Ala Val Phe Val Ser Ala Arg Tyr Gln Tyr Asn Val Leu Pro Gln Gly Ala Ser Arg Glu Leu
Glu Arg Phe Ala Val Asn Pro Gly Leu Leu Trp Ile Ile Leu Gly Leu Asn Lys Ile Val Arg Met Tyr
Ser Pro Thr Ser Ile Ala Ala Arg Thr Leu Asn Ala Trp Val Lys Val Phe Leu Trp Met Gly Tyr Glu
Leu His Leu Thr Phe Gly Trp Cys Phe Lys Leu Pro Leu Trp Lys Gly Pro Ala Lys Leu Val Thr
Val Tyr Tyr Gly Val Pro Val Ala Ala Leu Leu Trp Lys Gly Glu Gly Ala Val Ala Ala Ala Lys Leu
Val Gly Lys Leu Asn Trp Ala Lys Leu Leu Trp Lys Gly Glu Gly Ala Thr Leu Asn Phe Pro Ile
Ser Pro Ile Trp Gln Ala Thr Trp Ile Pro Glu Trp Lys Ala Ala Cys Trp Trp Ala Gly Ile Arg Gln
Ala Asn Phe Leu Gly Lys Ile Trp Pro Ser His Lys Gly Arg Asn Val Trp Ala Thr His Ala Cys Val
Ala Ala Glu Met Met Thr Ala Cys Gln Gly Val Ser Thr Val Gln Cys Thr His Gly Ile Ala Ala Lys
Gln Met Ala Gly Asp Asp Cys Val Ala Ala Trp Gln Leu Asp Cys Thr His Leu Glu Tyr Lys Ala
Ala Val Asp Leu Ser His Phe Leu Arg Glu Lys Gly Gly Leu Glu Gly Ala Ala Tyr Tyr Met Asp
Asp Leu Tyr Val Gly Ser Gly Gln Val Asp Cys Ser Pro Gly Ile Ala Thr Leu Glu Glu Met Met Thr
Ala Glu Leu His Pro Asp Lys Trp Thr Val Trp Thr Val Asn Asp Ile Gln Lys Leu Gly Ile Trp Gly
Cys Ser Gly Lys Leu Thr Val Asn Asp Ile Gln Lys Leu Val Ile Val Thr Asp Ser Gln Tyr Ala Leu
Tyr Val Asp Arg Phe Tyr Lys Thr Leu Tyr Val Asp Arg Phe Tyr Lys Thr Leu Arg Ala Glu Gln
Ala Ser Gln Glu Val Asp Leu Asn Thr Met Leu Asn Thr Val Lys Leu Thr Pro Leu Cys Val Thr
Leu Tyr Gln Tyr Met Asp Asp Leu Tyr Val Val Ile Tyr Gln Tyr Met Asp Asp Leu Trp Ile Ile Leu
Gly Leu Asn Lys Ile
(SEQ ID NO: 434)
Met Asn Pro Ser Ala Ala Val Ile Phe Cys Leu Ile Leu Leu Gly Leu
Ser Gly Thr Gln Gly Ile Leu Asp Met Ala Gln Pro Val Gly Ile Asn Thr Ser Thr Thr Cys Cys Tyr
Arg Phe Ile Asn Lys Lys Ile Pro Lys Gln Arg Leu Glu Ser Tyr Arg Arg Thr Thr Ser Ser His Cys
Pro Arg Glu Ala Val Ile Phe Lys Thr Lys Leu Asp Lys Glu Ile Cys Ala Asp Pro Thr Gln Lys Trp
Val Gln Asp Phe Met Lys His Leu Asp Lys Lys Thr Gln Thr Pro Lys Leu Ala Ser Ala Gly Ala
Tyr Gln Tyr Asn Val Leu Pro Gln Gly Ala Ser Arg Glu Leu Glu Arg Phe Ala Val Asn Pro Gly
Leu Leu Trp Ile Ile Leu Gly Leu Asn Lys Ile Val Arg Met Tyr Ser Pro Thr Ser Ile Ala Ala Arg
Thr Leu Asn Ala Trp Val Lys Val Phe Leu Trp Met Gly Tyr Glu Leu His Leu Thr Phe Gly Trp
Cys Phe Lys Leu Pro Leu Trp Lys Gly Pro Ala Lys Leu Val Thr Val Tyr Tyr Gly Val Pro Val Ala
Ala Leu Leu Trp Lys Gly Glu Gly Ala Val Ala Ala Ala Lys Leu Val Gly Lys Leu Asn Trp Ala
Lys Leu Leu Trp Lys Gly Glu Gly Ala Thr Leu Asn Phe Pro Ile Ser Pro Ile Trp Gln Ala Thr Trp
Ile Pro Glu Trp Lys Ala Ala Cys Trp Trp Ala Gly Ile Arg Gln Ala Asn Phe Leu Gly Lys Ile Trp
Pro Ser His Lys Gly Arg Asn Val Trp Ala Thr His Ala Cys Val Ala Ala Glu Met Met Thr Ala
Cys Gln Gly Val Ser Thr Val Gln Cys Thr His Gly Ile Ala Ala Lys Gln Met Ala Gly Asp Asp Cys
Val Ala Ala Trp Gln Leu Asp Cys Thr His Leu Glu Tyr Lys Ala Ala Val Asp Leu Ser His Phe
Leu Arg Glu Lys Gly Gly Leu Glu Gly Ala Ala Tyr Tyr Met Asp Asp Leu Tyr Val Gly Ser Gly
Gln Val Asp Cys Ser Pro Gly Ile Ala Thr Leu Glu Glu Met Met Thr Ala Glu Leu His Pro Asp Lys
Trp Thr Val Trp Thr Val Asn Asp Ile Gln Lys Leu Gly Ile Trp Gly Cys Ser Gly Lys Leu Thr Val
Asn Asp Ile Gln Lys Leu Val Ile Val Thr Asp Ser Gln Tyr Ala Leu Tyr Val Asp Arg Phe Tyr Lys
Thr Leu Tyr Val Asp Arg Phe Tyr Lys Thr Leu Arg Ala Glu Gln Ala Ser Gln Glu Val Asp Leu
Asn Thr Met Leu Asn Thr Val Lys Leu Thr Pro Leu Cys Val Thr Leu Tyr Gln Tyr Met Asp Asp
Leu Tyr Val Val Ile Tyr Gln Tyr Met Asp Asp Leu Trp Ile Ile Leu Gly Leu Asn Lys Ile
(SEQ ID NO: 435)
Met Arg Lys Ala Ala Val Ser His Trp Gln Gln Gln Ser Tyr Leu Asp
Ser Gly Ile His Ser Gly Ala Thr Thr Thr Ala Pro Ser Leu Ser Tyr Gln Tyr Asn Val Leu Pro Gln
Gly Ala Ser Arg Glu Leu Glu Arg Phe Ala Val Asn Pro Gly Leu Leu Trp Ile Ile Leu Gly Leu Asn
Lys Ile Val Arg Met Tyr Ser Pro Thr Ser Ile Ala Ala Arg Thr Leu Asn Ala Trp Val Lys Val Phe
Leu Trp Met Gly Tyr Glu Leu His Leu Thr Phe Gly Trp Cys Phe Lys Leu Pro Leu Trp Lys Gly
Pro Ala Lys Leu Val Thr Val Tyr Tyr Gly Val Pro Val Ala Ala Leu Leu Trp Lys Gly Glu Gly Ala
Val Ala Ala Ala Lys Leu Val Gly Lys Leu Asn Trp Ala Lys Leu Leu Trp Lys Gly Glu Gly Ala
Thr Leu Asn Phe Pro Ile Ser Pro Ile Trp Gln Ala Thr Trp Ile Pro Glu Trp Lys Ala Ala Cys Trp
Trp Ala Gly Ile Arg Gln Ala Asn Phe Leu Gly Lys Ile Trp Pro Ser His Lys Gly Arg Asn Val Trp
Ala Thr His Ala Cys Val Ala Ala Glu Met Met Thr Ala Cys Gln Gly Val Ser Thr Val Gln Cys
Thr His Gly Ile Ala Ala Lys Gln Met Ala Gly Asp Asp Cys Val Ala Ala Trp Gln Leu Asp Cys
Thr His Leu Glu Tyr Lys Ala Ala Val Asp Leu Ser His Phe Leu Arg Glu Lys Gly Gly Leu Glu
Gly Ala Ala Tyr Tyr Met Asp Asp Leu Tyr Val Gly Ser Gly Gln Val Asp Cys Ser Pro Gly Ile Ala
Thr Leu Glu Glu Met Met Thr Ala Glu Leu His Pro Asp Lys Trp Thr Val Trp Thr Val Asn Asp
Ile Gln Lys Leu Gly Ile Trp Gly Cys Ser Gly Lys Leu Thr Val Asn Asp Ile Gln Lys Leu Val Ile
Val Thr Asp Ser Gln Tyr Ala Leu Tyr Val Asp Arg Phe Tyr Lys Thr Leu Tyr Val Asp Arg Phe
Tyr Lys Thr Leu Arg Ala Glu Gln Ala Ser Gln Glu Val Asp Leu Asn Thr Met Leu Asn Thr Val
Lys Leu Thr Pro Leu Cys Val Thr Leu Tyr Gln Tyr Met Asp Asp Leu Tyr Val Val Ile Tyr Gln
Tyr Met Asp Asp Leu Trp Ile Ile Leu Gly Leu Asn Lys Ile
(SEQ ID NO: 436)
Asp Tyr Lys Asp Asp Asp Asp Lys Leu
(SEQ ID NO: 437)
Met Arg Val Lys Glu Lys Tyr Gln His Leu Trp Arg Trp Gly Trp Arg
Trp Gly Thr Met Leu Leu Gly Met Leu Met Ile
(SEQ ID NO: 438)
Phe Cys Ala Ser Asp Ala
(SEQ ID NO: 439)
Val Val Leu Val Asn Val Thr Glu Asn Phe Asn Met Trp Lys Asn
Asp Met Val Glu Gln Met His Glu Asp Ile Ile Ser Leu Trp
(SEQ ID NO: 440)
Asn Thr Asn Ser Ser Ser Gly Arg Met Ile Met Glu Lys Gly Glu Ile
Lys Asn Cys Ser Phe Asn Ile Ser Thr Ser Ile Arg Gly Lys Val Gln Lys Glu Tyr Ala Phe Phe Tyr
Lys Leu Asp Ile Ile Pro Ile Asp Asn Asp Thr Thr Ser Tyr Lys Leu Thr Ser Cys Asn Thr Ser Val
Ile Thr Gln Ala Cys Pro Lys Val Ser Phe Glu Pro Ile Pro Ile His Tyr Cys Ala Pro Ala Gly Phe
Ala Ile Leu Lys Cys Asn Asn Lys Thr Phe Asn
(SEQ ID NO: 441)
Glu Val Val Ile Arg Ser Val Asn Phe Thr Asp Asn Ala Lys Thr Ile
Ile Val Gln Leu Asn Thr Ser Val Glu Ile Asn Cys Thr Arg Pro Asn Asn Asn Thr Arg Lys Arg Ile
Arg Ile Gln Arg Gly Pro Gly Arg Ala Phe Val Thr Ile Gly Lys Ile Gly Asn Met Arg Gln Ala His
Cys Asn Ile Ser Arg Ala Lys Trp Asn Asn Thr Leu Lys Gln Ile Ala Ser Lys Leu Arg Glu Gln Phe
Gly Asn Asn Lys Thr Ile Ile Phe Lys Gln Ser Ser Gly Gly Asp Pro Glu Ile Val Thr His Ser Phe
Asn Cys Gly Gly Glu Phe Phe Tyr Cys Asn Ser Thr Gln Leu Phe Asn Ser Thr Trp Phe Asn Ser
Thr Trp Ser Thr Glu Gly Ser Asn Asn Thr Glu Gly Ser Asp Thr Ile Thr Leu Pro Cys Arg Ile Lys
Gln Ile Ile Asn Met Trp Gln Lys Val Gly Lys Ala Met Tyr Ala Pro Pro Ile Ser Gly Gln Ile Arg
Cys Ser Ser Asn Ile Thr Gly Leu Leu Leu Thr Arg Asp Gly Gly Asn Ser Asn Asn Glu Ser Glu Ile
Phe Arg Pro Gly Gly Gly Asp
(SEQ ID NO: 442)
Lys Ile Glu Pro Leu Gly Val Ala Pro Thr Lys Ala
(SEQ ID NO: 443)
Asn Ala Ser Trp Ser Asn Lys Ser Leu Glu Gln Ile Trp Asn His Thr
Thr Trp Met Glu Trp Asp Arg Glu Ile Asn Asn Tyr Thr Ser Leu Ile His Ser Leu Ile Glu Glu Ser
Gln Asn Gln Gln Glu Lys Asn Glu Gln Glu Leu Leu Glu Leu Asp Lys Trp Ala Ser Leu Trp Asn
Trp Phe Asn Ile Thr Asn Trp Leu Trp Tyr Ile Lys Leu Phe Ile Met Ile Val Gly Gly Leu Val Gly
Leu Arg Ile Val Phe Ala Val Leu Ser Ile Val Asn Arg Val Arg Gln Gly Tyr Ser Pro Leu Ser Phe
Gln Thr His Leu Pro Thr Pro Arg Gly Pro Asp Arg Pro Glu Gly Ile Glu Glu Glu Gly Gly Glu Arg
Asp Arg Asp Arg Ser Ile Arg Leu Val Asn Gly Ser Leu Ala Leu Ile Trp Asp Asp Leu Arg Ser
Leu Cys Leu Phe Ser Tyr His Arg Leu Arg Asp Leu Leu Leu Ile Val Thr Arg Ile Val Glu Leu Leu
Gly Arg Arg Gly Trp Glu Ala Leu Lys Tyr Trp Trp Asn Leu Leu Gln Tyr Trp Ser Gln Glu Leu
Lys Asn Ser Ala Val Ser Leu Leu Asn Ala Thr Ala Ile Ala Val Ala Glu Gly Thr Asp Arg Val Ile
Glu Val Val Gln Gly Ala Cys Arg Ala Ile Arg His Ile Pro Arg Arg Ile Arg Gln Gly Leu Glu Arg
Ile Leu Leu
(SEQ ID NO: 444)
Met Gly Ala Arg Ala Ser Val Leu Ser Gly Gly Glu Leu Asp Arg Trp
Glu Lys Ile Arg Leu Arg Pro Gly Gly Lys Lys Lys Tyr Lys
(SEQ ID NO: 445)
Ser Glu Gly Cys Arg Gln Ile Leu Gly Gln Leu Gln Pro Ser Leu Gln
Thr Gly Ser Glu Glu Leu Arg Ser Leu Tyr Asn Thr Val Ala Thr Leu Tyr Cys Val His Gln Arg Ile
Glu Ile Lys Asp Thr Lys Glu Ala Leu Asp Lys Ile Glu Glu Glu Gln Asn Lys Ser Lys Lys Lys Ala
Gln Gln Ala Ala Ala Asp Thr Gly His Ser Asn Gln
(SEQ ID NO: 446)
Ile Gln Gly Gln Met Val His Gln Ala
(SEQ ID NO: 447)
Val Thr Asn Ser Ala Thr Ile Met Met Gln Arg Gly Asn Phe Arg Asn
Gln Arg Lys Ile Val Lys Cys Phe Asn Cys Gly Lys Glu Gly His Thr Ala Arg Asn Cys Arg Ala
Pro Arg Lys Lys Gly Cys Trp Lys Cys Gly Lys Glu Gly His Gln Met Lys Asp Cys Thr Glu
(SEQ ID NO: 448)
Pro Gly Asn Phe Leu Gln Ser Arg Pro Glu Pro Thr Ala Pro Pro Glu
Glu Ser Phe Arg Ser Gly Val Glu Thr Thr Thr Pro Pro Gln Lys Gln Glu Pro Ile Asp Lys Glu Leu
Tyr Pro Leu Thr Ser Leu Arg Ser Leu Phe Gly Asn Asp Pro Ser Ser Gln
(SEQ ID NO: 449)
Met Gly Gly Lys Trp Ser Lys Ser Ser Val Ile Gly Trp Pro Thr Val
Arg Glu Arg Met Arg Arg Ala Glu Pro Ala Ala Asp Arg Val Gly Ala Ala Ser Arg Asp Leu Glu
Lys His Gly Ala Ile Thr Ser Ser Asn Thr Ala Ala Thr Asn Ala Ala Cys Ala Trp Leu Glu Ala Gln
Glu Glu
(SEQ ID NO: 450)
Leu Ile His Ser Gln Arg Arg Gln Asp Ile Leu Asp Leu Trp Ile Tyr
His
(SEQ ID NO: 451)
Pro Gly Val Arg Tyr Pro Leu Thr Phe Gly Trp Cys Tyr Lys Leu Val
Pro Val Glu Pro Asp Lys Ile Glu Glu Ala Asn Lys Gly Glu Asn Thr Ser Leu Leu His Pro Val Ser
Leu His Gly Met Asp Asp Pro Glu Arg Glu Val Leu Glu Trp Arg Phe Asp Ser Arg Leu Ala Phe
His His Val Ala Arg Glu Leu His Pro Glu Tyr Phe Lys Asn Cys
(SEQ ID NO: 452)
Phe Phe Arg Glu Asp Leu Ala Phe Leu Gln Gly Lys Ala Arg Glu Phe
Ser Ser Glu Gln Thr Arg Ala Asn Ser Pro Thr Arg Arg Glu Leu Gln Val Trp Gly Arg Asp Asn
Asn Ser Pro Ser Glu Ala Gly Ala Asp Arg Gln Gly Thr Val Ser Phe Asn
(SEQ ID NO: 453)
Ile Leu Ile Glu Ile Cys Gly His Lys Ala Ile
(SEQ ID NO: 454)
Lys Ile Leu Glu Pro
(SEQ ID NO: 455)
Thr Lys Ile Glu Glu Leu Arg Gln His Leu Leu Arg
(SEQ ID NO: 456)
Gln Leu Cys Lys Leu Leu Arg Gly Thr Lys Ala Leu Thr Glu Val Ile
Pro Leu Thr Glu Glu Ala Glu Leu Glu Leu Ala Glu Asn Arg Glu Ile Leu Lys Glu Pro Val His Gly
Val Tyr Tyr Asp Pro Ser Lys Asp Leu Ile Ala Glu Ile Gln Lys Gln Gly Gln Gly Gln Trp Thr Tyr
Gln Ile Tyr Gln Glu Pro Phe Lys Asn Leu Lys Thr Gly Lys Tyr Ala Arg Met Arg Gly Ala His Thr
Asn Asp Val Lys Gln Leu Thr Glu Ala Val Gln Lys Ile Thr Thr Glu Ser Ile Val Ile Trp Gly Lys
Thr
(SEQ ID NO: 457)
Leu Gly Lys Ala Gly Tyr Val Thr Asn Arg Gly Arg Gln Lys Val Val
Thr Leu Thr Asp Thr Thr Asn Gln Lys Thr Glu Leu Gln Ala Ile Tyr Leu Ala Leu
(SEQ ID NO: 458)
Gln Ser Glu Ser Glu Leu Val Asn Gln Ile Ile Glu Gln Leu Ile Lys
(SEQ ID NO: 459)
Gly Ile Arg Lys Val Leu Phe Leu Asp Gly Ile Asp Lys Ala Gln Asp
Glu His Glu Lys Tyr His Ser Asn Trp Arg Ala Met Ala Ser Asp Phe Asn Leu Pro Pro Val
(SEQ ID NO: 460)
Ile His Thr Asp Asn Gly
(SEQ ID NO: 461)
Thr Asp Ile Gln Thr Lys Glu Leu Gln Lys
(SEQ ID NO: 462)
Tyr Met Asp Asp
(SEQ ID NO: 463)
Tyr Val Asp Asp
(SEQ ID NO: 464)
Ala Ile Ile Ile Ile Ile Ile Ile Ser
(SEQ ID NO: 465)
Met Ile Ile Ile Ile Ile Ile Ile Ile
(SEQ ID NO: 466)
Ile Ile Ile Ile Ile Ile Ile Ser Lys
(SEQ ID NO: 467)
Ile Ile Ile Ile Ile Ile Ile Ile Arg
(SEQ ID NO: 468)
Ala Ile Ile Ile Ile Ile Ile Ile Ser Lys
(SEQ ID NO: 469)
Met Ile Ile Ile Ile Ile Ile Ile Ile Arg
(SEQ ID NO: 470)
Ala Ala Ala Ala, wherein SEQ ID NO: 470 contains a miscellaneous
feature from (1) . . . (4). SEQ ID NO: 470 may encompass 2-4 residues.
(SEQ ID NO: 471)
Gly Ile Ile Ile Ile Ile Ile Ile Ile Lys
(SEQ ID NO: 472)
Ala Ile Ile Ile Ile Ile Ile Ile Ile His
(SEQ ID NO: 473)
Gly Ile Ile Ile Ile Ile Ile Ile Ile His
(SEQ ID NO: 474)
Ala Ile Ile Ile Ile Ile Ile Ile Ile Lys
(SEQ ID NO: 475)
Gln Asn Leu Gln Gly Gln Met Val His Gln Ala Ile Ser Pro Arg Thr
Leu Asn Ala Trp Val Lys Val Val Lys Ala Phe
(SEQ ID NO: 476)
Gln Asn Leu Gln Gly Gln Met Val His Gln Ala Ile Ser Pro Arg Thr
Leu Asn Ala Trp Val Lys Val Glu Lys Ala Phe
(SEQ ID NO: 477)
Gln Asn Leu Gln Gly Gln Met Val His Gln Ala Ile Ser Pro Arg Thr
Leu Asn Ala Trp Val Lys Ile Val Lys Ala Phe
(SEQ ID NO: 478)
Gln Asn Ile Gln Gly Gln Met Val His Gln Ala Ile Ser Pro Arg Thr
Leu Asn Ala Trp Val Lys Ile Glu Lys Ala Phe
(SEQ ID NO: 479)
Gln Asn Ile Gln Gly Gln Met Val His Gln Ala Ile Ser Pro Arg Thr
Leu Asn Ala Trp Val Lys Val Val Lys Ala Phe
(SEQ ID NO: 480)
Pro Asn Ile Gln Gly Gln Met Val His Gln Ala Ile Ser Pro Arg Thr
Leu Asn Ala Trp Val Lys Ile Val Lys Ala Phe
(SEQ ID NO: 481)
Gln Asn Leu Gln Gly Gln Met Val His
(SEQ ID NO: 482)
Gln Asn Ile Gln Gly Gln Met Val His
(SEQ ID NO: 483)
Pro Asn Ile Gln Gly Gln Met Val His
(SEQ ID NO: 484)
Gly Ile Ile Ile Ile Ile Ile Ile Ile Arg
(SEQ ID NO: 485)
Gly Ile Ile Ile Ile Ile Ile Ile Ile
(SEQ ID NO: 486)
Ala Ile Ile Ile Ile Ile Ile Ile Ile
(SEQ ID NO: 487)
Ile Ile Ile Ile Ile Ile Ile Ile Lys
(SEQ ID NO: 488)
Ile Ile Ile Ile Ile Ile Ile Ile His
(SEQ ID NO: 489)
Gln Ala Ile Ser Pro Arg Thr Leu Asn
(SEQ ID NO: 490)
Gln Asn Leu Gln Gly Gln Met Val His Gln Ala Thr Ser Pro Arg Thr
Leu Asn Ala Trp Val Lys Val Val Lys Ala Phe
(SEQ ID NO: 491)
Gln Ala Thr Ser Pro Arg Thr Leu Asn
(SEQ ID NO: 492)
His Pro Pro Gln Ala Gly Pro Val Ala
(SEQ ID NO: 493)
Val Pro Leu Gln Ala Gly Pro Val Gln
(SEQ ID NO: 494)
Met Pro Gly Gln Ala Gly Pro Val Gly
(SEQ ID NO: 495)
Gln Tyr Arg Gln Ala Gly Pro Val Ile
(SEQ ID NO: 496)
Thr Ala Thr Gln Ala Gly Leu Val Phe
(SEQ ID NO: 497)
His Thr Lys Gln Ala Gly Leu Val Val
(SEQ ID NO: 498)
Gly Thr Arg Gln Ala Gly Leu Val Ala
(SEQ ID NO: 499)
Ile Ser Pro Arg Thr Leu Asn Ala Trp
(SEQ ID NO: 500)
Met Phe Ser Ala Leu Ser Glu Gly Ala
(SEQ ID NO: 501)
Ile Leu Asp Ile Arg Gln Gly Pro Lys
(SEQ ID NO: 502)
Leu Ser Pro Arg Thr Leu Asn Ala Trp
(SEQ ID NO: 503)
Met Phe Ser Ala Leu Ser Tyr Gly Ala
(SEQ ID NO: 504)
Ile Leu Asp Ile Arg Arg Gly Pro Lys
(SEQ ID NO: 505)
Ala Thr Leu Glu Glu Met Val Thr Ala
(SEQ ID NO: 506)
Ile Leu Asp Thr Arg Gln Gly Pro Lys
(SEQ ID NO: 507)
Ala Thr Leu Val Glu Met Met Thr Ala
(SEQ ID NO: 508)
Ile Leu Ser Ile Arg Gln Gly Pro Lys
(SEQ ID NO: 509)
Ala Thr Ser Glu Glu Met Met Thr Ala
(SEQ ID NO: 510)
Ile Leu Asp Ile Arg Gln Gly Xaa Lys, wherein Xaa is any amino acid
(SEQ ID NO: 511)
Lys Arg Arg Val Val Gln Arg Glu Lys Arg Ala Val Gly Ile Gly Ala
Met Phe Leu Gly Phe Leu Gly Ala Ala Gly Ser Thr Met Gly Ala Ala Ser Ile Thr Leu Thr Val Gln
Ala Arg Gln Leu Leu Ser Gly Ile Val Gln Gln Gln Asn Asn Leu Leu Arg Ala Ile Glu Ala Gln Gln
His Leu Leu Gln Leu Thr Val Trp Gly Ile Lys Gln Leu Gln Ala Arg Val Leu Ala Val Glu Arg Tyr
Leu Lys Asp Gln Gln Leu Leu Gly Ile Trp Gly Cys Ser Gly Lys Leu Ile Cys Thr Thr Ala Val Pro
Trp
(SEQ ID NO: 512)
Tyr Leu Lys Asp Gln Gln Leu Leu Gly Ile Trp Gly Cys Ser Gly Lys
Leu Ile Cys Thr Thr
(SEQ ID NO: 513)
Glu Glu Val Gly Phe Pro Val Lys Pro Gln Val Pro Leu Arg Pro Met
Thr Phe Lys Gly Ala Leu Asp Leu Ser His Phe Leu Arg Glu Lys Gly Gly Leu Glu
(SEQ ID NO: 514)
Thr Gln Gly Phe Phe Pro Asp Trp Gln Asn Tyr Thr Pro Glu
(SEQ ID NO: 515)
Pro Gly Ile Arg Phe Pro Leu Thr Phe Gly Trp Cys Phe Lys Leu Val
Pro Leu
(SEQ ID NO: 516)
Val Thr Val Leu Asp Val Gly Asp Ala Tyr Phe Ser Val Pro Leu Asp
Lys Asp Phe Arg Lys
(SEQ ID NO: 517)
Tyr Thr Ala Phe Thr Ile Pro Ser
(SEQ ID NO: 518)
Val Ile Tyr Gln Tyr Met Asp Asp
(SEQ ID NO: 519)
Thr Val Lys Ala Ala Cys Trp Trp Ala Gly Ile Lys Gln Glu Phe Gly
Ile Pro Tyr Asn Pro Gln Ser Gln Gly Val Val Glu Ser Met Asn Lys Glu Leu Lys Lys Ile Ile Gly
Gln Val Arg Asp Gln Ala Glu His Leu Lys Thr Ala Val Gln Met Ala Val Phe Ile His Asn Phe Lys
Arg Lys Gly Gly Ile Gly Gly Tyr Ser Ala Gly Glu Arg Ile Val Asp

TABLE 11
ChAd Vector Sequences

| SEQ ID | | |
|---|---|---|
| NO: | Description | Sequence |
| 520 | ChAd-01 | catcatcaataatatacctcaaactttttgtgcgcgttaatatgcaaatgaggcgtttgaatttgggg |
| | (underline, | aggaagggcggtgattggtcgagggatgagcgaccgttaggggggggcgagtgacgtttt |
| | bold, | gatgacgtggttgcgaggaggagccagtttgcaagttctcgtgggaaaagtgacgtcaaacga |
| | uppercase = | ggtgtggtttgaacacggaaatactcaattttcccgcgctctctgacaggaaatgaggtgtttctg |
| | Immunogen | ggcggatgcaagtgaaaacgggccattttcgcgcgaaaactgaatgaggaagtgaaaatctg |
| | (nt 1256 to | agtaatttcgcgtttatggcagggaggagtatttgccgagggccgagtagactttgaccgattac |
| | 4351); | gtgggggtttcgattaccgtgtttttcacctaaatttccgcgtacggtgtcaaagtccggtgttttta |
| | Italics, bold, | cgtaggtgtcagctgatcgccagggtatttaaacctgcgctctccagtcaagaggccactcttga |
| | uppercase = | gtgccagcgagaagagttttctcctccgcgccgcgagtcagatctacactttgaaagtagggat |
| | CMV | aacagggtaatGACATTGATTATTGACTAGTTGTTAATAGTAATC |
| | promoter (nt | AATTACGGGGTCATTAGTTCATAGCCCATATATGGAGTTCC |
| | 594 to 1195); | GCGTTACATAACTTACGGTAAATGGCCCGCCTGGCTGACC |
| | italics, dashed | GCCCAACGACCCCCGCCCATTGACGTCAATAATGACGTAT |
| | underline, | GTTCCCATAGTAACGCCAATAGGGACTTTCCATTGACGTCA |
| | bold = polyA | ATGGGTGGAGTATTTACGGTAAACTGCCCACTTGGCAGTA |
| | (nt 4396 to | CATCAAGTGTATCATATGCCAAGTCCGCCCCCTATTGACGT |
| | 4619) | CAATGACGGTAAATGGCCCGCCTGGCATTATGCCCAGTAC |
| | | ATGACCTTACGGGACTTTCCTACTTGGCAGTACATCTACGT |
| | | ATTAGTCATCGCTATTACCATGGTGATGCGGTTTTGGCAGT |
| | | ACACCAATGGGCGTGGATAGCGGTTTGACTCACGGGGATT |
| | | TCCAAGTCTCCACCCCATTGACGTCAATGGGAGTTTGTTTT |
| | | GGCACCAAAATCAACGGGACTTTCCAAAATGTCGTAATAA |
| | | CCCCGCCCCGTTGACGCAAATGGGCGGTAGGCGTGTACGG |
| | | TGGGAGGTCTATATAAGCAGAGCTCGTTTAGTGAACCGTC |
| | | AGatcgcctggaacgccatccacgctgttttgacctccatagaagacagegatcgcgccacc |
| | | ATGATTTCTCCTCGGACGCTGAATGCATGGGTGAAGG |
| | | TAGTGGAGGAAAAGGCATTTTCACCAGAAGTCATTCC |
| | | GATGTTCTCCGCGCTCTCCGAGGGTGCTACGCCACAG |
| | | GACTTGAATACGATGCTGAATACCGTTGGTGGCCATC |
| | | AGGCGGCGATGCAGATGTTGAAGGAGACAATTAACGA |
| | | AGAAGCCGCCGAATGGGACAGATTGCACCCGGTGCAT |
| | | GCGGGGCCAATAGCTCCTGGCCAGATGCGCGAGCCTA |
| | | GGGGTTCTGACATTGCTGGTACAACAAGTACCCTTCA |
| | | GGAGCAGATTGGTTGGATGACGAATAACCCTCCCATA |
| | | CCTGTTGGCGAGATCTATAAGCGCTGGATTATACTTG |
| | | GGCTGAATAAGATAGTCCGAATGTATTCTCCCACCTC |
| | | TATTCTGGATATTCGGCAAGGACCTAAGGAGCCGTTT |
| | | AGAGACTACGTAGACCGGTTTTACAAAACCCTGCGGG |
| | | CGGAACAAGCTTCTCAGGAAGTCAAAAATTGGATGAC |
| | | TGAGACCTTGCTCGTCCAGAATGCGAACCCGGACTGT |
| | | AAAACAATACTCAAAGCGCTGGGCCCCGCTGCAACCC |
| | | TGGAAGAAATGATGACGGCTTGTCAGGGAGTAGGAGG |
| | | CCCCGGACATAAGGCACGAGTGTTGGCAGAAGCCATG |
| | | AGCCAGCCGCCTGTCGTCGCGAAAGAAATCGTCGCTT |
| | | CTTGCGACAAATGTCAGCTGAAGGGGGAGGCGATGCA |
| | | CGGTCAAGTTGATTGCTCTCCCGGTATTTGGCAATTG |
| | | GACTGTACCCACCTTGAAGGCAAAATTATTCTGGTTG |
| | | CAGTGCACGTAGCATCCGGTTACATCGAAGCTGAAGT |
| | | GATACCCGCAGAGACAGGCCAGGAGACGGCTTATTTC |
| | | CTCCTTAAGCTTGCGGGTCGGTGGCCCGTAAAGACCA |
| | | TGGCTGCTCGGGCATCTGTCCTCTCCGGTGGTGAACT |
| | | CGACCGATGGGAAAAGATTCGATTGCGCCCCGGAGGA |
| | | AAGAAGAAATATAGGCTGAAACATATTGTGTGGGCAT |
| | | CACGGGAACTTGAGCGATTTGCGGTAAACCCAGGCCT |
| | | TTTGGAAACAGCTGCAACTGTGAAAGCGGCTTGCTGG |
| | | TGGGCGGGGATAAAACAGGAGTTTGGTATCCCGTACA |
| | | ATCCCCAATCTCAGGGGGTAGTAGAAAGCATGAACAA |
| | | AGAATTGAAAAAAATAATTGGCCAGGTTCGCGACCAA |
| | | GCCGAGCACCTCAAGACCGCTGTACAGATGGCTGTAT |
| | | TTATTCACAACTTCAAGCGGAAGGGCGGAATAGGAGG |
| | | ATATAGCGCAGGGGAAAGGATTGTTGATATTATTGCA |
| | | ACACAAGGTTACTTTCCTGACTGGCAAAACTACACAC |
| | | CGGGCCCTGGCACGCGCTATCCCCTTACGTTCGGTTG |
| | | GTGCTTCAAGCTGGTGCCGGTAAAAGAAAAAGTTTAT |
| | | TTGGCATGGGTTCCTGCACATAAAGGAATAGGGGGTA |
| | | ACGAACAAGTTGACAAACTCGTCAGCCGCGCTAAAAG |
| | | AGCCCCAGTCAAGCAGACCCTGAATTTTGACCTGCTT |
| | | AAATTGGCTGGGGACGTCGAGAGTAACCCGGGACCCT |
| | | TCCCACAAATTACACTCTGGCAGCGACCACTGGTAAC |
| | | AATCAAAATAGGGGGACAATTGAAAGAAGCACTCCTG |
| | | GATACGGGCGCGGACGATACAGTCCTGGAGGAAATGA |
| | | ATCTCCCCGGCCGCTGGAAACCTAAGATGATAGGGGG |
| | | GATCGGTGGATTTATTAAAGTGCGGCAGTACGATCAA |
| | | TGGGGTTTTACGACACCTGACAAGAAACATCAAAAGG |
| | | AGCCGCCATTTCTTTGGATGGGTTATGAGCTTCATCC |
| | | GGATAAATGGACTGTTCAGCCGATTGTCCTCCCCGAG |
| | | AAGGATAGTTGGACTGTGAACGACATCCAGAAGCTGG |
| | | TCGGGAAACTTAATTGGGCCAGTCAAATATATCCAGG |
| | | TATTAAAGTTGCCGCACAAGAAGAGGAGGAAGTAGGG |
| | | TTCCCCGTGAAACCGCAAGTCCCTCTCCGGCCCATGA |
| | | CCTTTAAGGGCGCTCTCGACCTGTCCCATTTCCTCCG |
| | | CGAAAAGGGGGGTTTGGAAGGCTTGATCATTACCAAG |
| | | ATTCAAAACTTCAGGGTCTATTATCGAGACAGTCGCG |
| | | ATCCCCTTTGGAAAGGACCTGCGAAACTTCTTTGGAA |
| | | AGGAGAAGGAGCCGTGGTAATTCAGGATAATTCTGAC |
| | | ATAAAGGTCGTCCCACGCCGAAAAGCGAAGATTATAA |
| | | GAGATTATGGCAAGCAGATGGCCGGGGATGATTGTGT |
| | | CGCAAGTAGACAGGATGAAGACCCTAAATTCAAGCTT |
| | | CCAATCCAGAAGGAAACGTGGGAGACATGGTGGACCG |
| | | AGTATTGGCAAGCTACTTGGATCCCAGAATGGGAATT |
| | | TGTGAACACTCCCCCACTCGTAAAGCTGTGGTACCAG |
| | | CTTGAAAAAGAACCTATAGTCGGGGCGGAGACGTTCT |
| | | ATGTAGACGGCGCCGCTAATCGAGAGACAAAAGCAGC |
| | | AGGCACGGTACTGGTAGGCCCGACCCCTGTCAACATC |
| | | ATTGGACGAAATCTGTTGACCCAGATTGGGTGTACCC |
| | | TTAACTTTCCCATTTCACCAATAGAGACCGTCCCGGTT |
| | | AAACTGAAACCGGGTATGGATGGTCCCAAAGTAAAAC |
| | | AGTGGCCACTTACCGAGGAGAAGATTAAGGCACTCGT |
| | | TGAAATATGTACAGAAATGGAAAAAGAGGGGAAAATC |
| | | TCTAAAATTGGCCCTGAAAATCCGTACAACACTCCGG |
| | | TATTCGCCATAAAAAAGAAGGACTCTACCAAGTGGCG |
| | | CAAACTCGTGGACTTTAGAGAACTGAATAAAAGGACC |
| | | CAGGACTTTTGGGAAGTCCAGCTGGGTATTCCTCACC |
| | | CCGCTGGTCTCAAGAAAAAGAAAAGTGTCACTGTCTT |
| | | GGATGTTGGAGATGCGTACTTTTCAGTACCTCTTGAT |
| | | AAAGATTTTCGAAAGTATACCGCGTTTACCATTCCCTC |
| | | CATAAATAACGAAACACCGGGGATCAGGTATCAATAT |
| | | AACGTGCTTCCACAAGGCTGGAAGGGTTCACCGGCTA |
| | | TTTTCCAATCTTCTATGACGTGA |
| | | 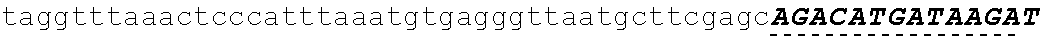 |
| | | 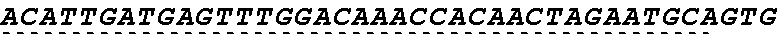 |
| | | 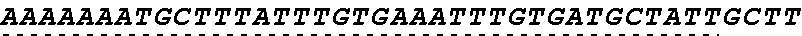 |
| | | 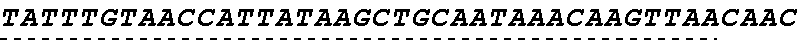 |
| | | 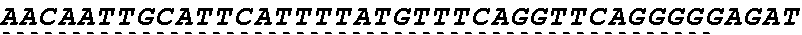 |
| | | 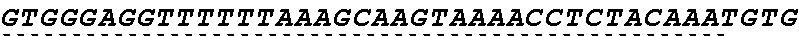 |
| | | 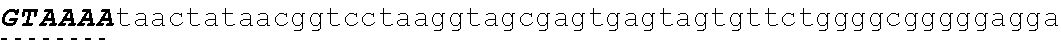 |
| | | cctgcatgagggccagaataactgaaatctgtgcttttctgtgtgttgcagcagcatgagcggaa |
| | | gcggctcctttgagggaggggtattcagcccttatctgacggggcgtctcccctcctgggcgg |
| | | gagtgcgtcagaatgtgatgggatccacggtggacggccggcccgtgcagcccgcgaactct |
| | | tcaaccctgacctatgcaaccctgagctcttcgtcgttggacgcagctgccgccgcagctgctg |
| | | catctgccgccagcgccgtgcgcggaatggccatgggcgccggctactacggcactctggtg |
| | | gccaactcgagttccaccaataatcccgccagcctgaacgaggagaagctgttgctgctgatg |
| | | gcccagctcgaggccttgacccagcgcctgggcgagctgacccagcaggtggctcagctgc |
| | | aggagcagacgcgggccgcggttgccacggtgaaatccaaataaaaaatgaatcaataaata |
| | | aacggagacggttgttgattttaacacagagtctgaatctttatttgatttttcgcgcgcggtaggc |
| | | cctggaccaccggtctcgatcattgagcacccggtggatcttttccaggacccggtagaggtgg |
| | | gcttggatgttgaggtacatgggcatgagcccgtcccgggggtggaggtagctccattgcagg |
| | | gcctcgtgctcgggggtggtgttgtaaatcacccagtcatagcaggggcgcagggcatggtgt |
| | | tgcacaatatctttgaggaggagactgatggccacgggcagccctttggtgtaggtgtttacaaa |
| | | tctgttgagctgggagggatgcatgcggggggagatgaggtgcatcttggcctggatcttgag |
| | | attggcgatgttaccgcccagatcccgcctggggttcatgttgtgcaggaccaccagcacggtg |
| | | tatccggtgcacttggggaatttatcatgcaacttggaagggaaggcgtgaaagaatttggcga |
| | | cgcctttgtgcccgcccaggttttccatgcactcatccatgatgatggcgatgggcccgtgggc |
| | | ggcggcctgggcaaagacgtttcgggggtcggacacatcatagttgtggtcctgggtgaggtc |
| | | atcataggccattttaatgaatttggggcggagggtgccggactgggggacaaaggtaccctc |
| | | gatcccgggggcgtagttcccctcacagatctgcatctcccaggctttgagctcggagggggg |
| | | gatcatgtccacctgcggggcgataaagaacacggtttccggggcgggggagatgagctgg |
| | | gccgaaagcaagttccggagcagctgggacttgccgcagccggtggggccgtagatgaccc |
| | | cgatgaccggctgcaggtggtagttgagggagagacagctgccgtcctcccggaggagggg |
| | | ggccacctcgttcatcatctcgcgcacgtgcatgttctcgcgcaccagttccgccaggaggcgc |
| | | tctccccccagggataggagctcctggagcgaggcgaagtttttcagcggcttgagtccgtcg |
| | | gccatgggcattttggagagggtttgttgcaagagttccaggcggtcccagagctcggtgatgt |
| | | gctctacggcatctcgatccagcagacctcctcgtttcgcgggttgggacggctgcgggagta |
| | | gggcaccagacgatgggcgtccagcgcagccagggtccggtccttccagggtcgcagegtc |
| | | cgcgtcagggtggtctccgtcacggtgaaggggtgcgcgccgggctgggcgcttgcgaggg |
| | | tgcgcttcaggctcatccggctggtcgaaaaccgctcccgatcggcgccctgcgcgtcggcca |
| | | ggtagcaattgaccatgagttcgtagttgagcgcctcggccgcgtggcctttggcgcggagctt |
| | | acctttggaagtctgcccgcaggcgggacagaggagggacttgagggcgtagagcttgggg |
| | | gcgaggaagacggactcgggggcgtaggcgtccgcgccgcagtgggcgcagacggtctcg |
| | | cactccacgagccaggtgaggtcgggctggtcggggtcaaaaaccagtttcccgccgttctttt |
| | | tgatgcgtttcttacctttggtctccatgagctcgtgtccccgctgggtgacaaagaggctgtccg |
| | | tgtccccgtagaccgactttatgggccggtcctcgagcggtgtgccgcggtcctcctcgtagag |
| | | gaaccccgcccactccgagacgaaagcccgggtccaggccagcacgaaggaggccacgtg |
| | | ggacgggtagcggtcgttgtccaccagcgggtccaccttttccagggtatgcaaacacatgtcc |
| | | ccctcgtccacatccaggaaggtgattggcttgtaagtgtaggccacgtgaccgggggtcccg |
| | | gccgggggggtataaaagggtgcgggtccctgctcgtcctcactgtcttccggatcgctgtcca |
| | | ggagcgccagctgttggggtaggtattccctctcgaaggcgggcatgacctcggcactcaggt |
| | | tgtcagtttctagaaacgaggaggatttgatattgacggtgccggcggagatgcctttcaagagc |
| | | ccctcgtccatctggtcagaaaagacgatctttttgttgtcgagcttggtggcgaaggagccgta |
| | | gagggcgttggagaggagcttggcgatggagcgcatggtctggtttttttccttgtcggcgcgct |
| | | ccttggcggcgatgttgagctgcacgtactcgcgcgccacgcacttccattcggggaagacgg |
| | | tggtcagctcgtcgggcacgattctgacctgccagccccgattatgcagggtgatgaggtccac |
| | | actggtggccacctcgccgcgcaggggctcattagtccagcagaggcgtccgcccttgcgcg |
| | | agcagaaggggggcagggggtccagcatgacctcgtcgggggggtcggcatcgatggtga |
| | | agatgccgggcaggaggtcggggtcaaagtagctgatggaagtggccagatcgtccagggc |
| | | agcttgccattcgcgcacggccagcgcgcgctcgtagggactgaggggcgtgccccagggc |
| | | atgggatgggtaagcgcggaggcgtacatgccgcagatgtcgtagacgtagaggggctcctc |
| | | gaggatgccgatgtaggtggggtagcagcgccccccgcggatgctggcgcgcacgtagtca |
| | | tacagctcgtgcgagggggcgaggagccccgggcccaggttggtgcgactgggcttttcggc |
| | | gcggtagacgatctggcggaaaatggcatgcgagttggaggagatggtgggcctttggaaga |
| | | tgttgaagtgggcgtggggcagtccgaccgagtcgcggatgaagtgggcgtaggagtcttgc |
| | | agcttggcgacgagctcggcggtgactaggacgtccagagcgcagtagtcgagggtctcctg |
| | | gatgatgtcatacttgagctgtcccttttgtttccacagctcgcggttgagaaggaactcttcgcg |
| | | gtccttccagtactcttcgagggggaacccgtcctgatctgcacggtaagagcctagcatgtag |
| | | aactggttgacggccttgtaggcgcagcagcccttctccacggggagggcgtaggcctgggc |
| | | ggccttgcgcagggaggtgtgcgtgagggcgaaagtgtccctgaccatgaccttgaggaact |
| | | ggtgcttgaagtcgatatcgtcgcagcccccctgctcccagagctggaagtccgtgcgcttctt |
| | | gtaggcggggttgggcaaagcgaaagtaacatcgttgaagaggatcttgcccgcgcggggc |
| | | ataaagttgcgagtgatgcggaaaggttggggcacctcggcccggttgttgatgacctgggcg |
| | | gcgagcacgatctcgtcgaagccgttgatgttgtggcccacgatgtagagttccacgaatcgcg |
| | | gacggcccttgacgtggggcagtttcttgagctcctcgtaggtgagctcgtcggggtcgctgag |
| | | cccgtgctgctcgagcgcccagtcggcgagatgggggttggcgcggaggaaggaagtcca |
| | | gagatccacggccagggcggtttgcagacggtcccggtactgacggaactgctgcccgacg |
| | | gccattttttcgggggtgacgcagtagaaggtgcgggggtccccgtgccagcgatcccatttga |
| | | gctggagggcgagatcgagggcgagctcgacgagccggtcgtccccggagagtttcatgac |
| | | cagcatgaaggggacgagctgcttgccgaaggaccccatccaggtgtaggtttccacatcgta |
| | | ggtgaggaagagcctttcggtgcgaggatgcgagccgatggggaagaactggatctcctgcc |
| | | accaattggaggaatggctgttgatgtgatggaagtagaaatgccgacggcgcgccgaacact |
| | | cgtgcttgtgtttatacaagcggccacagtgctcgcaacgctgcacgggatgcacgtgctgcac |
| | | gagctgtacctgagttcctttgacgaggaatttcagtgggaagtggagtcgtggcgcctgcatct |
| | | cgtgctgtactacgtcgtggtggtcggcctggccctcttctgcctcgatggtggtcatgctgacg |
| | | agcccgcgcgggaggcaggtccagacctcggcgcgagcgggtcggagagcgaggacga |
| | | gggcgcgcaggccggagctgtccagggtcctgagacgctgcggagtcaggtcagtgggca |
| | | gcggcggcgcgcggttgacttgcaggagtttttccagggcgcgcgggaggtccagatggtac |
| | | ttgatctccaccgcgccattggtggcgacgtcgatggcttgcagggtcccgtgcccctggggtg |
| | | tgaccaccgtcccccgtttcttcttgggcggctggggcgacgggggcggtgcctcttccatggt |
| | | tagaagcggcggcgaggacgcgcgccgggcggcaggggcggctcggggcccggaggca |
| | | ggggggcaggggcacgtcggcgccgcgcgcgggtaggttctggtactgcgcccggagaa |
| | | gactggcgtgagcgacgacgcgacggttgacgtcctggatctgacgcctctgggtgaaggcc |
| | | acgggacccgtgagtttgaacctgaaagagagttcgacagaatcaatctcggtatcgttgacgg |
| | | cggcctgccgcaggatctcttgcacgtcgcccgagttgtcctggtaggcgatctcggtcatgaa |
| | | ctgctcgatctcctcctcttgaaggtctccgcggccggcgcgctccacggtggccgcgaggtc |
| | | gttggagatgcggcccatgagctgcgagaaggcgttcatgcccgcctcgttccagacgcggct |
| | | gtagaccacgacgccctcgggatcgcgggcgcgcatgaccacctgggcgaggttgagctcc |
| | | acgtggcgcgtgaagaccgcgtagttgcagaggcgctggtagaggtagttgagcgtggtggc |
| | | gatgtgctcggtgacgaagaaatacatgatccagcggcggagcggcatctcgctgacgtcgc |
| | | ccagcgcctccaaacgttccatggcctcgtaaaagtccacggcgaagttgaaaaactgggagt |
| | | tgcgcgccgagacggtcaactcctcctccagaagacggatgagctcggcgatggtggcgcg |
| | | cacctegcgctcgaaggcccccgggagttcctccacttcctcttcttcctcctccactaacatctc |
| | | ttctacttcctcctcaggcggcagtggtggcgggggagggggcctgcgtcgccggcggcgca |
| | | cgggcagacggtcgatgaagcgctcgatggtctcgccgcgccggcgtcgcatggtctcggtg |
| | | acggcgcgcccgtcctcgcggggccgcagcgtgaagacgccgccgcgcatctccaggtgg |
| | | ccgggggggtccccgttgggcagggagagggcgctgacgatgcatcttatcaattgccccgt |
| | | agggactccgcgcaaggacctgagcgtctcgagatccacgggatctgaaaaccgctgaacg |
| | | aaggcttcgagccagtcgcagtcgcaaggtaggctgagcacggtttcttctgggggtcatgtt |
| | | ggttgggagcggggcgggcgatgctgctggtgatgaagttgaaataggcggttctgagacgg |
| | | cggatggtggcgaggagcaccaggtctttgggcccggcttgctggatgcgcagacggtcggc |
| | | catgccccaggcgtggtcctgacacctggccaggtccttgtagtagtcctgcatgagccgctcc |
| | | acgggcacctcctcctcgcccgcgcggccgtgcatgcgcgtgagcccgaagccgcgctggg |
| | | gctggacgagcgccaggtcggcgacgacgcgctcggcgaggatggcttgctggatctgggt |
| | | gagggtggtctggaagtcatcaaagtcgacgaagcggtggtaggctccggtgttgatggtgta |
| | | ggagcagttggccatgacggaccagttgacggtctggtggcccggacgcacgagctcgtggt |
| | | acttgaggcgcgagtaggcgcgcgtgtcgaagatgtagtcgttgcaggtgcgcaccaggtact |
| | | ggtagccgatgaggaagtgcggcggcggctggcggtagagcggccatcgctcggtggcgg |
| | | gggcgccgggcgcgaggtcctcgagcatggtgcggtggtagccgtagatgtacctggacatc |
| | | caggtgatgccggcggcggtggtggaggcgcgcgggaactcgcggacgcggttccagatgt |
| | | tgcgcagcggcaggaagtagttcatggtgggcacggtctggcccgtgaggcgcgcgcagtc |
| | | gtggatgctctatacgggcaaaaacgaaagcggtcagcggctcgactccgtggcctggaggc |
| | | taagcgaacgggttgggctgcgcgtgtaccccggttcgaatctcgaatcaggctggagccgca |
| | | gctaacgtggtattggcactcccgtctcgacccaagcctgcaccaaccctccaggatacggag |
| | | gcgggtcgttttgcaacttttttttggaggccggatgagactagtaagcgcggaaagcggccga |
| | | ccgcgatggctcgctgccgtagtctggagaagaatcgccagggttgcgttgcggtgtgccccg |
| | | gttcgaggccggccggattccgcggctaacgagggcgtggctgccccgtcgtttccaagacc |
| | | ccatagccagccgacttctccagttacggagcgagcccctcttttgttttgtttgtttttgccagatg |
| | | catcccgtactgeggcagatgcgcccccaccaccctccaccgcaacaacagccccctccaca |
| | | gccggcgcttctgcccccgccccagcagcaacttccagccacgaccgccgcggccgccgtg |
| | | agcggggctggacagagttatgatcaccagctggccttggaagagggcgaggggctggcgc |
| | | gcctgggggcgtcgtcgccggagcggcacccgcgcgtgcagatgaaaagggacgctcgcg |
| | | aggcctacgtgcccaagcagaacctgttcagagacaggagcggcgaggagcccgaggaga |
| | | tgcgcgcggcccggttccacgcggggcgggagctgcggcgcggcctggaccgaaagagg |
| | | gtgctgagggacgaggatttcgaggcggacgagctgacggggatcagccccgcgcgcgcg |
| | | cacgtggccgcggccaacctggtcacggcgtacgagcagaccgtgaaggaggagagcaac |
| | | ttccaaaaatccttcaacaaccacgtgcgcaccctgatcgcgcgcgaggaggtgaccctgggc |
| | | ctgatgcacctgtgggacctgctggaggccatcgtgcagaaccccaccagcaagccgctgac |
| | | ggcgcagctgttcctggtggtgcagcatagtcgggacaacgaagcgttcagggaggcgctgc |
| | | tgaatatcaccgagcccgagggccgctggctcctggacctggtgaacattctgcagagcatcg |
| | | tggtgcaggagcgcgggctgccgctgtccgagaagctggcggccatcaacttctcggtgctg |
| | | agtttgggcaagtactacgctaggaagatctacaagaccccgtacgtgcccatagacaaggag |
| | | gtgaagatcgacgggttttacatgcgcatgaccctgaaagtgctgaccctgagcgacgatctgg |
| | | gggtgtaccgcaacgacaggatgcaccgtgcggtgagcgccagcaggcggcgcgagctga |
| | | gcgaccaggagctgatgcatagtctgcagcgggccctgaccggggccgggaccgaggggg |
| | | agagctactttgacatgggcgcggacctgcactggcagcccagccgccgggccttggaggc |
| | | ggcggcaggaccctacgtagaagaggtggacgatgaggtggacgaggagggcgagtacct |
| | | ggaagactgatggcgcgaccgtatttttgctagatgcaacaacaacagccacctcctgatcccg |
| | | cgatgcgggcggcgctgcagagccagccgtccggcattaactcctcggacgattggacccag |
| | | gccatgcaacgcatcatggcgctgacgacccgcaaccccgaagcctttagacagcagcccca |
| | | ggccaaccggctctcggccatcctggaggccgtggtgccctegcgctccaaccccacgcacg |
| | | agaaggtcctggccatcgtgaacgcgctggtggagaacaaggccatccgcggcgacgaggc |
| | | cggcctggtgtacaacgcgctgctggagcgcgtggcccgctacaacagcaccaacgtgcag |
| | | accaacctggaccgcatggtgaccgacgtgcgcgaggccgtggcccagcgcgagcggttcc |
| | | accgcgagtccaacctgggatccatggtggcgctgaacgccttcctcagcacccagcccgcc |
| | | aacgtgccccggggccaggaggactacaccaacttcatcagcgccctgcgcctgatggtgac |
| | | cgaggtgccccagagcgaggtgtaccagtccgggccggactacttcttccagaccagtcgcc |
| | | agggcttgcagaccgtgaacctgagccaggctttcaagaacttgcagggcctgtggggcgtg |
| | | caggccccggtcggggaccgcgcgacggtgtcgagcctgctgacgccgaactcgcgcctgc |
| | | tgctgctgctggtggcccccttcacggacagcggcagcatcaaccgcaactcgtacctgggct |
| | | acctgattaacctgtaccgcgaggccatcggccaggcgcacgtggacgagcagacctaccag |
| | | gagatcacccacgtgagccgcgccctgggccaggacgacccgggcaacctggaagccacc |
| | | ctgaactttttgctgaccaaccggtcgcagaagatcccgccccagtacgcgctcagcaccgag |
| | | gaggagcgcatcctgcgttacgtgcagcagagcgtgggcctgttcctgatgcaggagggggc |
| | | cacccccagcgcegcgctcgacatgaccgcgcgcaacatggagcccagcatgtacgccagc |
| | | aaccgcccgttcatcaataaactgatggactacttgcatcgggcggccgccatgaactctgact |
| | | atttcaccaacgccatcctgaatccccactggctcccgccgccggggttctacacgggcgagt |
| | | acgacatgcccgaccccaatgacgggttcctgtgggacgatgtggacagcagcgtgttctccc |
| | | cccgaccgggtgctaacgagcgccccttgtggaagaaggaaggcagcgaccgacgcccgt |
| | | cctcggcgctgtccggccgcgagggtgctgccgcggcggtgcccgaggccgccagtcctttc |
| | | ccgagcttgcccttctcgctgaacagtatccgcagcagcgagctgggcaggatcacgcgccc |
| | | gcgcttgctgggcgaagaggagtacttgaatgactcgctgttgagacccgagcgggagaaga |
| | | acttccccaataacgggatagaaagcctggtggacaagatgagccgctggaagacgtatgcg |
| | | caggagcacagggacgatccccgggcgtcgcagggggccacgagccggggcagcgccg |
| | | cccgtaaacgccggtggcacgacaggcagcggggacagatgtgggacgatgaggactccg |
| | | ccgacgacagcagcgtgttggacttggggggagtggtaacccgttcgctcacctgegccccc |
| | | gtatcgggcgcatgatgtaagagaaaccgaaaataaatgatactcaccaaggccatggcgacc |
| | | agcgtgcgttcgtttcttctctgttgttgttgtatctagtatgatgaggcgtgcgtacccggagggt |
| | | cctcctccctcgtacgagagcgtgatgcagcaggcgatggcggcggcggcgatgcagcccc |
| | | cgctggaggctccttacgtgcccccgcggtacctggcgcctacggaggggggaacagcatt |
| | | cgttactcggagctggcacccttgtacgataccacccggttgtacctggtggacaacaagtcgg |
| | | cggacatcgcctcgctgaactaccagaacgaccacagcaacttcctgaccaccgtggtgcag |
| | | aacaatgacttcacccccacggaggccagcacccagaccatcaactttgacgagcgctcgcg |
| | | gtggggcggccagctgaaaaccatcatgcacaccaacatgcccaacgtgaacgagttcatgta |
| | | cagcaacaagttcaaggcgcgggtgatggtctcccgcaagacccccaatggggtgacagtga |
| | | cagaggattatgatggtagtcaggatgagctgaagtatgaatgggtggaatttgagctgcccga |
| | | aggcaacttctcggtgaccatgaccatcgacctgatgaacaacgccatcatcgacaattacttg |
| | | gcggtggggcggcagaacggggtgctggagagcgacatcggcgtgaagttcgacactagg |
| | | aacttcaggctgggctgggaccccgtgaccgagctggtcatgcccggggtgtacaccaacga |
| | | ggctttccatcccgatattgtcttgctgcccggctgcggggtggacttcaccgagagccgcctc |
| | | agcaacctgctgggcattcgcaagaggcagcccttccaggaaggcttccagatcatgtacgag |
| | | gatctggaggggggcaacatccccgcgctcctggatgtcgacgcctatgagaaaagcaagga |
| | | ggatgcagcagctgaagcaactgcagccgtagctaccgcctctaccgaggtcaggggcgata |
| | | attttgcaagcgccgcagcagtggcagcggccgaggcggctgaaaccgaaagtaagatagtc |
| | | attcagccggtggagaaggatagcaagaacaggagctacaacgtactaccggacaagataaa |
| | | caccgcctaccgcagctggtacctagcctacaactatggcgaccccgagaagggcgtgcgct |
| | | cctggacgctgctcaccacctcggacgtcacctgcggcgtggagcaagtctactggtcgctgc |
| | | ccgacatgatgcaagacccggtcaccttccgctccacgcgtcaagttagcaactacccggtgg |
| | | tgggcgccgagctcctgcccgtctactccaagagcttcttcaacgagcaggccgtctactcgca |
| | | gcagctgcgcgccttcacctcgcttacgcacgtcttcaaccgettccccgagaaccagatcctc |
| | | gtccgcccgcccgcgcccaccattaccaccgtcagtgaaaacgttcctgctctcacagatcac |
| | | gggaccctgccgctgcgcagcagtatccggggagtccagcgcgtgaccgttactgacgcca |
| | | gacgccgcacctgcccctacgtctacaaggccctgggcatagtcgcgccgcgcgtcctctcg |
| | | agccgcaccttctaaatgtccattctcatctcgcccagtaataacaccggttggggcctgcgcgc |
| | | gcccagcaagatgtacggaggcgctcgccaacgctccacgcaacaccccgtgcgcgtgcgc |
| | | gggcacttccgcgctccctggggcgccctcaagggccgcgtgcggtcgcgcaccaccgtcg |
| | | acgacgtgatcgaccaggtggtggccgacgcgcgcaactacacccccgccgccgcgcccgt |
| | | ctccaccgtggacgccgtcatcgacagcgtggtggccgacgcgcgccggtacgcccgcgcc |
| | | aagagccggcggcggcgcatcgcccggcggcaccggagcacccccgccatgcgcgcggc |
| | | gcgagccttgctgcgcagggccaggcgcacgggacgcagggccatgctcagggcggcca |
| | | gacgcgcggcttcaggcgccagcgccggcaggacccggagacgcgcggccacggcggc |
| | | ggcagcggccatcgccagcatgtcccgcccgcggcgagggaacgtgtactgggtgcgcga |
| | | cgccgccaccggtgtgcgcgtgcccgtgcgcacccgcccccctcgcacttgaagatgttcact |
| | | tcgcgatgttgatgtgtcccagcggcgaggaggatgtccaagcgcaaattcaaggaagagatg |
| | | ctccaggtcatcgcgcctgagatctacggccctgcggtggtgaaggaggaaagaaagccccg |
| | | caaaatcaagcgggtcaaaaaggacaaaaaggaagaagaaagtgatgtggacggattggtg |
| | | gagtttgtgcgcgagttcgccccccggcggcgcgtgcagtggcgcgggcggaaggtgcaac |
| | | cggtgctgagacccggcaccaccgtggtcttcacgcccggcgagcgctccggcaccgcttcc |
| | | aagcgctcctacgacgaggtgtacggggatgatgatattctggagcaggcggccgagcgcct |
| | | gggcgagtttgcttacggcaagcgcagccgttccgcaccgaaggaagaggcggtgtccatcc |
| | | cgctggaccacggcaaccccacgccgagcctcaagcccgtgaccttgcagcaggtgctgcc |
| | | gaccgcggcgccgcgccgggggttcaagcgcgagggcgaggatctgtaccccaccatgca |
| | | gctgatggtgcccaagcgccagaagctggaagacgtgctggagaccatgaaggtggacccg |
| | | gacgtgcagcccgaggtcaaggtgcggcccatcaagcaggtggccccgggcctgggcgtg |
| | | cagaccgtggacatcaagattcccacggagcccatggaaacgcagaccgagcccatgatcaa |
| | | gcccagcaccagcaccatggaggtgcagacggatccctggatgccatcggctcctagtcgaa |
| | | gaccccggcgcaagtacggcgcggccagcctgctgatgcccaactacgcgctgcatccttcc |
| | | atcatccccacgccgggctaccgcggcacgcgcttctaccgcggtcataccagcagccgccg |
| | | ccgcaagaccaccactcgccgccgccgtcgccgcaccgccgctgcaaccacccctgccgcc |
| | | ctggtgcggagagtgtaccgccgcggccgcgcacctctgaccctgccgegegegegctacc |
| | | acccgagcatcgccatttaaactttcgcctgctttgcagatcaatggccctcacatgccgccttcg |
| | | cgttcccattacgggctaccgaggaagaaaaccgcgccgtagaaggctggggggaacggg |
| | | atgcgtcgccaccaccaccggcggcggcgcgccatcagcaagcggttggggggaggcttcc |
| | | tgcccgcgctgatccccatcatcgccgcggcgatcggggcgatccccggcattgcttccgtgg |
| | | cggtgcaggcctctcagcgccactgagacacacttggaaacatcttgtaataaaccaatggact |
| | | ctgacgctcctggtcctgtgatgtgttttcgtagacagatggaagacatcaatttttcgtccctggc |
| | | tccgcgacacggcacgcggccgttcatgggcacctggagcgacatcggcaccagccaactg |
| | | aacgggggcgccttcaattggagcagtctctggagcgggcttaagaatttcgggtccacgctta |
| | | aaacctatggcagcaaggcgtggaacagcaccacagggcaggcgctgagggataagctga |
| | | aagagcagaacttccagcagaaggtggtcgatgggctcgcctcgggcatcaacggggggtg |
| | | gacctggccaaccaggccgtgcagcggcagatcaacagccgcctggacccggtgccgccc |
| | | gccggctccgtggagatgccgcaggtggaggaggagctgcctcccctggacaagcggggc |
| | | gagaagcgaccccgccccgatgcggaggagacgctgctgacgcacacggacgagccgcc |
| | | cccgtacgaggaggcggtgaaactgggtctgcccaccacgcggcccategcgcccctggcc |
| | | accggggtgctgaaacccgaaaagcccgcgaccctggacttgcctcctccccagcetteccg |
| | | cccctctacagtggctaagcccctgccgccggtggccgtggcccgcgcgcgacccgggggc |
| | | accgcccgccctcatgcgaactggcagagcactctgaacagcatcgtgggtctgggagtgca |
| | | gagtgtgaagcgccgccgctgctattaaacctaccgtagcgcttaacttgcttgtctgtgtgtgta |
| | | tgtattatgtcgccgccgccgctgtccaccagaaggaggagtgaagaggcgcgtcgccgagtt |
| | | gcaagatggccaccccatcgatgctgccccagtgggcgtacatgcacatcgccggacaggac |
| | | gcttcggagtacctgagtccgggtctggtgcagtttgcccgcgccacagacacctacttcagtct |
| | | ggggaacaagtttaggaaccccacggtggcgcccacgcacgatgtgaccaccgaccgcagc |
| | | cagcggctgacgctgcgcttcgtgcccgtggaccgcgaggacaacacctactcgtacaaagt |
| | | gcgctacacgctggccgtgggcgacaaccgcgtgctggacatggccagcacctactttgacat |
| | | ccgcggcgtgctggatcggggccctagcttcaaaccctactccggcaccgcctacaacagtct |
| | | ggcccccaagggagcacccaacacttgtcagtggacatataaagccgatggtgaaactgcca |
| | | cagaaaaaacctatacatatggaaatgcacccgtgcagggcattaacatcacaaaagatggtat |
| | | tcaacttggaactgacaccgatgatcagccaatctacgcagataaaacctatcagcctgaacct |
| | | caagtgggtgatgctgaatggcatgacatcactggtactgatgaaaagtatggaggcagagct |
| | | cttaagcctgataccaaaatgaagccttgttatggttcttttgccaagcctactaataaagaagga |
| | | ggtcaggcaaatgtgaaaacaggaacaggcactactaaagaatatgacatagacatggctttct |
| | | ttgacaacagaagtgcggctgctgctggcctagctccagaaattgttttgtatactgaaaatgtgg |
| | | atttggaaactccagatacccatattgtatacaaagcaggcacagatgacagcagctcttctatta |
| | | atttgggtcagcaagccatgcccaacagacctaactacattggtttcagagacaactttatcggg |
| | | ctcatgtactacaacagcactggcaatatgggggtgctggccggtcaggcttctcagctgaatg |
| | | ctgtggttgacttgcaagacagaaacaccgagctgtcctaccagctcttgcttgactctctgggt |
| | | gacagaacccggtatttcagtatgtggaatcaggcggtggacagctatgatcctgatgtgcgca |
| | | ttattgaaaatcatggtgtggaggatgaacttcccaactattgtttccctctggatgctgttggcag |
| | | aacagatacttatcagggaattaaggctaatggaactgatcaaaccacatggaccaaagatgac |
| | | agtgtcaatgatgctaatgagataggcaagggtaatccattcgccatggaaatcaacatccaag |
| | | ccaacctgtggaggaacttcctctacgccaacgtggccctgtacctgcccgactcttacaagta |
| | | cacgccggccaatgttaccctgcccaccaacaccaacacctacgattacatgaacggccgggt |
| | | ggtggcgccctcgctggtggactcctacatcaacatcggggcgcgctggtcgctggatcccat |
| | | ggacaacgtgaaccccttcaaccaccaccgcaatgcggggctgcgctaccgctccatgctcct |
| | | gggcaacgggcgctacgtgcccttccacatccaggtgccccagaaatttttcgccatcaagag |
| | | cctcctgctcctgcccgggtcctacacctacgagtggaacttccgcaaggacgtcaacatgatc |
| | | ctgcagagctccctcggcaacgacctgcgcacggacggggcctccatctccttcaccagcatc |
| | | aacctctacgccaccttcttccccatggcgcacaacacggcctccacgctcgaggccatgctg |
| | | cgcaacgacaccaacgaccagtccttcaacgactacctcteggcggccaacatgctctacccc |
| | | atcccggccaacgccaccaacgtgcccatctccatcccctegcgcaactgggccgccttccgc |
| | | ggctggtccttcacgcgtctcaagaccaaggagacgccctcgctgggctccgggttcgacccc |
| | | tacttcgtctactegggctccatcccctacctcgacggcaccttctacctcaaccacaccttcaag |
| | | aaggtctccatcaccttcgactcctccgtcagctggcccggcaacgaccggctcctgacgccc |
| | | aacgagttcgaaatcaagcgcaccgtcgacggcgagggctacaacgtggcccagtgcaacat |
| | | gaccaaggactggttcctggtccagatgctggcccactacaacateggctaccagggcttctac |
| | | gtgcccgagggctacaaggaccgcatgtactccttcttccgcaacttccagcccatgagccgc |
| | | caggtggtggacgaggtcaactacaaggactaccaggccgtcaccctggcctaccagcacaa |
| | | caactcgggcttcgtcggctacctcgcgcccaccatgcgccagggccagccctaccccgcca |
| | | actacccctacccgctcatcggcaagagcgccgtcaccagcgtcacccagaaaaagttcctct |
| | | gcgacagggtcatgtggcgcatccccttctccagcaacttcatgtccatgggcgcgctcaccga |
| | | cctcggccagaacatgctctatgccaactccgcccacgcgctagacatgaatttcgaagtcgac |
| | | cccatggatgagtccacccttctctatgttgtcttcgaagtcttcgacgtcgtccgagtgcaccag |
| | | ccccaccgcggcgtcategaggccgtctacctgcgcacccccttctcggccggtaacgccac |
| | | cacctaagctcttgcttcttgcaagccatggccgcgggctccggcgagcaggagctcagggc |
| | | catcatccgcgacctgggctgcgggccctacttcctgggcaccttcgataagcgcttcccggg |
| | | attcatggccccgcacaagctggcctgcgccatcgtcaacacggccggccgcgagaccggg |
| | | ggcgagcactggctggccttcgcctggaacccgcgctegaacacctgctacctettegacccc |
| | | ttcgggttctcggacgagcgcctcaagcagatctaccagttcgagtacgagggcctgctgcgc |
| | | cgcagcgccctggccaccgaggaccgctgcgtcaccctggaaaagtccacccagaccgtgc |
| | | agggtccgcgctcggccgcctgcgggctcttctgctgcatgttcctgcacgccttcgtgcactg |
| | | gcccgaccgccccatggacaagaaccccaccatgaacttgctgacgggggtgcccaacggc |
| | | atgctccagtcgccccaggtggaacccaccctgcgccgcaaccaggaggcgctctaccgctt |
| | | cctcaactcccactccgcctactttegctcccaccgegcgcgcatcgagaaggccacegccttc |
| | | gaccgcatgaatcaagacatgtaaaccgtgtgtgtatgttaaatgtctttaataaacagcactttca |
| | | tgttacacatgcatctgagatgatttatttagaaatcgaaagggttctgccgggtctcggcatggc |
| | | ccgcgggcagggacacgttgcggaactggtacttggccagccacttgaactcggggatcagc |
| | | agtttgggcagcggggtgtcggggaaggagtcggtccacagcttccgcgtcagttgcagggc |
| | | gcccagcaggtcgggcgcggagatcttgaaatcgcagttgggacccgcgttctgcgcgcgg |
| | | gagttgcggtacacggggttgcagcactggaacaccatcagggccgggtgcttcacgctcgc |
| | | cagcaccgtcgcgtcggtgatgctctccacgtcgaggtcctcggcgttggccatcccgaaggg |
| | | ggtcatcttgcaggtctgccttcccatggtgggcacgcacccgggcttgtggttgcaatcgcagt |
| | | gcagggggatcagcatcatctgggcctggtcggcgttcatccccgggtacatggccttcatga |
| | | aagcctccaattgcctgaacgcctgctgggccttggctccctcggtgaagaagaccccgcagg |
| | | acttgctagagaactggttggtggcgcacccggcgtcgtgcacgcagcagcgcgcgtcgttgt |
| | | tggccagctgcaccacgctgcgcccccagcggttctgggtgatcttggcccggtcggggttct |
| | | ccttcagcgcgcgctgcccgttctcgctcgccacatccatctcgatcatgtgctccttctggatca |
| | | tggtggtcccgtgcaggcaccgcagcttgccctcggcctcggtgcacccgtgcagccacagc |
| | | gcgcacccggtgcactcccagttcttgtgggcgatctgggaatgcgcgtgcacgaagccctgc |
| | | aggaagcggcccatcatggtggtcagggtcttgttgctagtgaaggtcagcggaatgccgcgg |
| | | tgctcctcgttgatgtacaggtggcagatgcggcggtacacctcgccctgctcgggcatcagct |
| | | ggaagttggctttcaggtcggtctccacgcggtagcggtccatcagcatagtcatgatttccata |
| | | cccttctcccaggccgagacgatgggcaggctcatagggttcttcaccatcatcttagcgctag |
| | | cagccgcggccagggggtcgctctcgtccagggtctcaaagctccgcttgccgtccttctcggt |
| | | gatccgcaccggggggtagctgaagcccacggccgccagctcctcctcggcctgtctttcgtc |
| | | ctcgctgtcctggctgacgtcctgcaggaccacatgcttggtcttgcggggtttcttcttgggcgg |
| | | cagcggcggcggagatgttggagatggcgagggggagcgcgagttctcgctcaccactact |
| | | atctcttcctcttcttggtccgaggccacgcggcggtaggtatgtctcttcgggggcagaggcg |
| | | gaggcgacgggctctcgccgccgcgacttggcggatggctggcagagccccttccgcgttcg |
| | | ggggtgcgctcccggcggcgctctgactgacttcctccgcggccggccattgtgttctcctagg |
| | | gaggaacaacaagcatggagactcagccatcgccaacctcgccatctgcccccaccgccga |
| | | cgagaagcagcagcagcagaatgaaagcttaaccgccccgccgcccagccccgccacctcc |
| | | gacgcggccgtcccagacatgcaagagatggaggaatccatcgagattgacctgggctatgt |
| | | gacgcccgcggagcacgaggaggagctggcagtgcgcttttcacaagaagagatacaccaa |
| | | gaacagccagagcaggaagcagagaatgagcagagtcaggctgggctcgagcatgacggc |
| | | gactacctccacctgaggggggggaggacgcgctcatcaagcatctggcccggcaggcca |
| | | ccatcgtcaaggatgcgctgctcgaccgcaccgaggtgcccctcagcgtggaggagctcagc |
| | | cgcgcctacgagttgaacctcttctcgccgcgcgtgccccccaagcgccagcccaatggcac |
| | | ctgcgagcccaacccgcgcctcaacttctacccggtcttegcggtgcccgaggccctggccac |
| | | ctaccacatctttttcaagaaccaaaagatccccgtctcctgccgcgccaaccgcacccgcgcc |
| | | gacgcccttttcaacctgggtcccggcgcccgcctacctgatatcgcctccttggaagaggttc |
| | | ccaagatcttcgagggtctgggcagcgacgagactcgggccgcgaacgctctgcaaggaga |
| | | aggaggagagcatgagcaccacagcgccctggtcgagttggaaggcgacaacgcgcggct |
| | | ggcggtgctcaaacgcacggtcgagctgacccatttcgcctacccggctctgaacctgccccc |
| | | caaagtcatgagcgcggtcatggaccaggtgctcatcaagcgcgcgtcgcccatctccgagg |
| | | acgagggcatgcaagactccgaggagggcaagcccgtggtcagcgacgagcagctggccc |
| | | ggtggctgggtcctaatgctagtccccagagtttggaagagcggcgcaaactcatgatggccg |
| | | tggtcctggtgaccgtggagctggagtgcctgcgccgcttcttcgccgacgcggagaccctgc |
| | | gcaaggtcgaggagaacctgcactacctcttcaggcacgggttcgtgcgccaggcctgcaag |
| | | atctccaacgtggagctgaccaacctggtctcctacatgggcatcttgcacgagaaccgcctgg |
| | | ggcagaacgtgctgcacaccaccctgcgcggggaggcccggcgcgactacatccgcgact |
| | | gcgtctacctctacctctgccacacctggcagacgggcatgggcgtgtggcagcagtgtctgg |
| | | aggagcagaacctgaaagagctctgcaagctcctgcagaagaacctcaagggtctgtggacc |
| | | gggttcgacgagcgcaccaccgcctcggacctggccgacctcattttccccgagcgcctcag |
| | | gctgacgctgcgcaacggcctgcccgactttatgagccaaagcatgttgcaaaactttcgctctt |
| | | tcatcctcgaacgctccggaatcctgcccgccacctgctccgcgctgccctcggacttcgtgcc |
| | | gctgaccttccgcgagtgccccccgccgctgtggagccactgctacctgctgcgcctggccaa |
| | | ctacctggcctaccactcggacgtgatcgaggacgtcagcggcgagggcctgctcgagtgcc |
| | | actgccgctgcaacctctgcacgccgcaccgctccctggcctgcaacccccagctgctgagc |
| | | gagacccagatcatcggcaccttcgagttgcaagggcccagcgaaggcgagggttcagccg |
| | | ccaaggggggtctgaaactcaccccggggctgtggacctcggcctacttgcgcaagttcgtgc |
| | | ccgaggactaccatcccttcgagatcaggttctacgaggaccaatcccatccgcccaaggccg |
| | | agctgtcggcctgcgtcatcacccagggggcgatcctggcccaattgcaagccatccagaaat |
| | | cccgccaagaattcttgctgaaaaagggccgcggggtctacctcgacccccagaccggtgag |
| | | gagctcaaccccggcttcccccaggatgccccgaggaaacaagaagctgaaagtggagctg |
| | | ccgcccgtggaggatttggaggaagactgggagaacagcagtcaggcagaggaggaggag |
| | | atggaggaagactgggacagcactcaggcagaggaggacagcctgcaagacagtctggag |
| | | gaagacgaggaggaggcagaggaggaggtggaagaagcagccgccgccagaccgtcgtc |
| | | ctcggcgggggagaaagcaagcagcacggataccatctccgctccgggtcggggtcccgct |
| | | cgaccacacagtagatgggacgagaccggacgattcccgaaccccaccacccagaccggta |
| | | agaaggagcggcagggatacaagtcctggcgggggcacaaaaacgccatcgtctcctgcttg |
| | | caggcctgcgggggcaacatctccttcacccggcgctacctgctcttccaccgcggggtgaac |
| | | tttccccgcaacatcttgcattactaccgtcacctccacagcccctactacttccaagaagaggc |
| | | agcagcagcagaaaaagaccagcagaaaaccagcagctagaaaatccacagcggcggcag |
| | | caggtggactgaggatcgcggcgaacgagccggcgcaaacccgggagctgaggaaccgg |
| | | atctttcccaccctctatgccatcttccagcagagtcgggggcaggagcaggaactgaaagtca |
| | | agaaccgttctctgcgctcgctcacccgcagttgtctgtatcacaagagcgaagaccaacttca |
| | | gcgcactctcgaggacgccgaggctctcttcaacaagtactgcgcgctcactcttaaagagtag |
| | | cccgcgcccgcccagtcgcagaaaaaggcgggaattacgtcacctgtgcccttcgccctagc |
| | | cgcctccacccatcatcatgagcaaagagattcccacgccttacatgtggagctaccagcccca |
| | | gatgggcctggccgccggtgccgcccaggactactccacccgcatgaattggctcagcgccg |
| | | ggcccgcgatgatctcacgggtgaatgacatccgcgcccaccgaaaccagatactcctagaa |
| | | cagtcagcgctcaccgccacgccccgcaatcacctcaatccgcgtaattggcccgccgccctg |
| | | gtgtaccaggaaattccccagcccacgaccgtactacttccgcgagacgcccaggccgaagt |
| | | ccagctgactaactcaggtgtccagctgggggcggcgccaccctgtgtcgtcaccgccccg |
| | | ctcagggtataaagcggctggtgatccggggcagaggcacacagctcaacgacgaggtggt |
| | | gagctcttcgctgggtctgcgacctgacggagtcttccaactcgccggatcggggagatcttcc |
| | | ttcacgcctcgtcaggccgtcctgactttggagagttcgtcctcgcagccccgctcgggtggca |
| | | tcggcactctccagttcgtggaggagttcactccctcggtctacttcaaccccttctceggctccc |
| | | ccggccactacccggacgagttcatcccgaacttcgacgccatcagcgagtcggtggacggc |
| | | tacgattgaaactaatcacccccttatccagtgaaataaagatcatattgatgatgattttacagaa |
| | | ataaaaaataatcatttgatttgaaataaagatacaatcatattgatgatttgagtttaacaaaaaaat |
| | | aaagaatcacttacttgaaatctgataccaggtctctgtccatgttttctgccaacaccacttcactc |
| | | ccctcttcccagctctggtactgcaggccccggcgggctgcaaacttcctccacacgctgaag |
| | | gggatgtcaaattcctcctgtccctcaatcttcattttatcttctatcagatgtccaaaaagcgcgtc |
| | | cgggtggatgatgacttcgaccccgtctacccctacgatgcagacaacgcaccgaccgtgccc |
| | | ttcatcaacccccccttcgtctcttcagatggattccaagagaagcccctgggggtgttgtccctg |
| | | cgactggccgaccccgtcaccaccaagaacggggaaatcaccctcaagctgggagagggg |
| | | gtggacctcgattcctcgggaaaactcatctccaacacggccaccaaggccgccgcccctctc |
| | | agtttttccaacaacaccatttcccttaacatggatcaccccttttacactaaagatggaaaattatc |
| | | cttacaagtttctccaccattaaatatactgagaacaagcattctaaacacactagctttaggttttg |
| | | gatcaggtttaggactccgtggctctgccttggcagtacagttagtctctccacttacatttgatact |
| | | gatggaaacataaagcttaccttagacagaggtttgcatgttacaacaggagatgcaattgaaa |
| | | gcaacataagctgggctaaaggtttaaaatttgaagatggagccatagcaaccaacattggaaa |
| | | tgggttagagtttggaagcagtagtacagaaacaggtgttgatgatgcttacccaatccaagtta |
| | | aacttggatctggccttagctttgacagtacaggagccataatggctggtaacaaagaagacga |
| | | taaactcactttgtggacaacacctgatccatcaccaaactgtcaaatactcgcagaaaatgatg |
| | | caaaactaacactttgcttgactaaatgtggtagtcaaatactggccactgtgtcagtcttagttgt |
| | | aggaagtggaaacctaaaccccattactggcaccgtaagcagtgctcaggtgtttctacgttttg |
| | | atgcaaacggtgttcttttaacagaacattctacactaaaaaaatactgggggtataggcaggga |
| | | gatagcatagatggcactccatataccaatgctgtaggattcatgcccaatttaaaagcttatcca |
| | | aagtcacaaagttctactactaaaaataatatagtagggcaagtatacatgaatggagatgtttca |
| | | aaacctatgcttctcactataaccctcaatggtactgatgacagcaacagtacatattcaatgtcat |
| | | tttcatacacctggactaatggaagctatgttggagcaacatttggggctaactcttataccttctc |
| | | atacatcgcccaagaatgaacactgtatcccaccctgcatgccaacccttcccaccccactctgt |
| | | ggaacaaactctgaaacacaaaataaaataaagttcaagtgttttattgattcaacagttttacagg |
| | | attcgagcagttatttttcctccaccctcccaggacatggaatacaccaccctetccccccgcac |
| | | agccttgaacatctgaatgccattggtgatggacatgcttttggtctccacgttccacacagtttca |
| | | gagcgagccagtctcgggtcggtcagggagatgaaaccctccgggcactcccgcatctgcac |
| | | ctcacagctcaacagctgaggattgtcctcggtggtcgggatcacggttatctggaagaagcag |
| | | aagagcggcggtgggaatcatagtccgcgaacgggatcggccggtggtgtcgcatcaggcc |
| | | ccgcagcagtcgctgccgccgccgctccgtcaagctgctgctcagggggtccgggtccagg |
| | | gactccctcagcatgatgcccacggccctcagcatcagtcgtctggtgcggcgggcgcagca |
| | | gcgcatgcggatctcgctcaggtcgctgcagtacgtgcaacacagaaccaccaggttgttcaa |
| | | cagtccatagttcaacacgctccagccgaaactcatcgcgggaaggatgctacccacgtggcc |
| | | gtcgtaccagatcctcaggtaaatcaagtggtgccccctccagaacacgctgcccacgtacatg |
| | | atctccttgggcatgtggcggttcaccacctcccggtaccacatcaccctctggttgaacatgca |
| | | gccccggatgatcctgcggaaccacagggccagcaccgccccgcccgccatgcagcgaag |
| | | agaccccgggtcccggcaatggcaatggaggacccaccgctcgtacccgtggatcatctggg |
| | | agctgaacaagtctatgttggcacagcacaggcatatgctcatgcatctcttcagcactctcaact |
| | | cctcgggggtcaaaaccatatcccagggcacggggaactcttgcaggacagcgaaccccgc |
| | | agaacagggcaatcctcgcacagaacttacattgtgcatggacagggtatcgcaatcaggcag |
| | | caccgggtgatcctccaccagagaagcgcgggtctcggtctcctcacagcgtggtaaggggg |
| | | ccggccgatacgggtgatggcgggacgcggctgatcgtgttcgcgaccgtgtcatgatgcagt |
| | | tgctttcggacattttcgtacttgctgtagcagaacctggtccgggcgctgcacaccgatcgccg |
| | | gcggcggtctcggcgcttggaacgctcggtgttgaaattgtaaaacagccactctctcagaccg |
| | | tgcagcagatctagggcctcaggagtgatgaagatcccatcatgcctgatggctctgatcacat |
| | | cgaccaccgtggaatgggccagacccagccagatgatgcaattttgttgggtttcggtgacgg |
| | | cgagcctcgggaacaacgatgaagtaaatgcaagcggtgcgttccagcatggttagttagctg |
| | | atctgtagaaaaaacaaaaatgaacattaaaccatgctagcctggcgaacaggtgggtaaatcg |
| | | ttctctccagcaccaggcaggccacggggtctccggcgcgaccctcgtaaaaattgtcgctatg |
| | | attgaaaaccatcacagagagacgttcccggtggccggcgtgaatgattcgacaagatgaata |
| | | cacccccggaacattggcgtccgcgagtgaaaaaaagcgcccgaggaagcaataaggcact |
| | | acaatgctcagtctcaagtccagcaaagcgatgccatgcggatgaagcacaaaattctcaggt |
| | | gcgtacaaaatgtaattactcccctcctgcacaggcagcaaagcccccgatccctccaggtaca |
| | | catacaaagcctcagcgtccatagcttaccgagcagcagcacacaacaggcgcaagagtcag |
| | | agaaaggctgagctctaacctgtccacccgctctctgctcaatatatagcccagatctacactga |
| | | cgtaaaggccaaagtctaaaaatacccgccaaataatcacacacgcccagcacacgcccaga |
| | | aaccggtgacacactcaaaaaaatacgcgcacttcctcaaacgcccaaaactgccgtcatttcc |
| | | gggttcccacgctacgtcatgaaaacacgactttcaaattccgtcgaccgttaaaaacgtcaccc |
| | | gccccgcccctaacggtcgcccgtctctcagccaatcagegccccgcatccccaaattcaaac |
| | | acctcatttgcatattaacgcgcacaaaaagtttgaggtatattattgatgatg |
| 521 | ChAd-02 | catcatcaataatatacctcaaactttttgtgcgcgttaatatgcaaatgaggcgtttgaatttggggaggaa |
| | (underline, | gggcggtgattggtcgagggatgagcgaccgttaggggcggggcgagtgacgttttgatgacgtggtt |
| | bold, | gcgaggaggagccagtttgcaagttctcgtgggaaaagtgacgtcaaacgaggtgtggtttgaacacg |
| | uppercase | gaaatactcaattttcccgcgctctctgacaggaaatgaggtgtttctgggcggatgcaagtgaaaacgg |
| | Immunogen | gccattttcgcgcgaaaactgaatgaggaagtgaaaatctgagtaatttcgcgtttatggcagggaggag |
| | (nt 1256 to | tatttgccgagggccgagtagactttgaccgattacgtgggggtttcgattaccgtgtttttcacctaaatttc |
| | 4348); italic, | cgcgtacggtgtcaaagtccggtgtttttacgtaggtgtcagctgatcgccagggtatttaaacctgcgctc |
| | bold, | tccagtcaagaggccactcttgagtgccagcgagaagagttttctcctccgcgccgcgagtcagatctac |
| | uppercase = | actttgaaagtagggataacagggtaatGACATTGATTATTGACTAGTTGTTAAT |
| | CMV | AGTAATCAATTACGGGGTCATTAGTTCATAGCCCATATATGGAG |
| | promoter (nt | TTCCGCGTTACATAACTTACGGTAAATGGCCCGCCTGGCTGACC |
| | 594 to 1195); | GCCCAACGACCCCCGCCCATTGACGTCAATAATGACGTATGTTC |
| | dashed | CCATAGTAACGCCAATAGGGACTTTCCATTGACGTCAATGGGTG |
| | underline, | GAGTATTTACGGTAAACTGCCCACTTGGCAGTACATCAAGTGTA |
| | bold, | TCATATGCCAAGTCCGCCCCCTATTGACGTCAATGACGGTAAAT |
| | lowercase = | GGCCCGCCTGGCATTATGCCCAGTACATGACCTTACGGGACTTT |
| | polyA (nt | CCTACTTGGCAGTACATCTACGTATTAGTCATCGCTATTACCATG |
| | 4393 to 4616) | GTGATGCGGTTTTGGCAGTACACCAATGGGCGTGGATAGCGGTT |
| | | TGACTCACGGGGATTTCCAAGTCTCCACCCCATTGACGTCAATG |
| | | GGAGTTTGTTTTGGCACCAAAATCAACGGGACTTTCCAAAATGT |
| | | CGTAATAACCCCGCCCCGTTGACGCAAATGGGCGGTAGGCGTGT |
| | | ACGGTGGGAGGTCTATATAAGCAGAGCTCGTTTAGTGAACCGTC |
| | | AGatcgcctggaacgccatccacgctgttttgacctccatagaagacagcgatcgcgccaccATG |
| | | TTGTCTCCTAGAACTCTGAATGCTTGGGTAAAGGTGATCGA |
| | | AGAAAAGGCATTCTCACCCGAGGTTATCCCTATGTTTACTG |
| | | CGTTGAGCGAAGGCGCAACACCCCATGATCTGAACACAAT |
| | | GCTGAATACAATCGGCGGACATCAAGCTGCTATGCAAATG |
| | | CTTAAGGACACCATCAATGAGGAGGCAGCCGAGTGGGATC |
| | | GCGTTCATCCAGTCCACGCTGGGCCCGTTGCGCCTGGTCA |
| | | GATGAGGGACCCACGAGGATCCGACATCGCAGGGAGCACC |
| | | AGTACACTCCAAGAGCAGATTGCATGGATGACGAACAATC |
| | | CACCAATACCTGTCGGTGACATTTACAAAAGGTGGATTATT |
| | | ATGGGCTTGAACAAAATTGTACGGATGTATAGCCCGGTGA |
| | | GCATACTGGACATTAAACAGGGTCCAAAAGAACCCTTTCGA |
| | | GATTACGTTGACAGATTTTATAGGACACTGAGGGCGGAAC |
| | | AAGCGTCTCAAGACGTTAAAAACTGGATGACAGAAACGCT |
| | | GCTTGTACAGAATTCCAACCCAGACTGCAAAACTATACTGA |
| | | AAGCGCTCGGTCCCGGTGCGACACTTGAGGAAATGATGAG |
| | | CGCATGCCAGGGCGTCGGAGGGCCGTCTCACAAGGCCCGC |
| | | GTGTTGGCGGAAGCTATGTGCCAGGTGGCTAAGGAGATAG |
| | | TAGCATGTTGTGATAAATGTCAGCTTAAAGGTGAAGCTATA |
| | | CATGGGCAGGTGGATTGTAGCCCGGGTGTATGGCAGCTGG |
| | | ACTGTACTCATCTGGAAGGAAAGGTAATACTTGTCGCAGTT |
| | | CATGTCGCGAGCGGATACATTGAAGCTGAAATCATTCCTAC |
| | | GGAGACGGGCCAAGAGACAGCTTACTTCATACTTAAACTTG |
| | | CTGGGCGATGGCCGGTGACAACTATGGCGGCGCGCGCTTC |
| | | AATTTTGAGTGGTGGAAAGTTGGACAAGTGGGAGAAGATT |
| | | AGACTCAGACCCGGAGGGAGAAAGAAGTATAAACTGAAAC |
| | | ATCTGGTTTGGGCTTCACGCGAACTTGAACGGTTTGCTCTC |
| | | AACCCCGGGCTGCTCGAAACTGCTGCTGCTGTAAAGGCCG |
| | | CTTGTTGGTGGGCGGGGGTAAAGCAGGAATTCGGAATTCC |
| | | ATATAACACTCAAAGCCAGGGAGTAGTGGAATCCATGAATA |
| | | ATGAACTTAAGAAGATAATTGGACAGATTCGCGATCAGGCT |
| | | GAACATCTCAAGACGGCCGTACAAATGGCAGTATTGATTCA |
| | | TAACTTTAAACGGAAGGGCGGCATAGGAGAGTATTCTGCG |
| | | GGAGAACGCATAATAGATATAATTGCGACTCAGGGGTTCTT |
| | | TCCGGATTGGCAGAACTATACGCCGGGGCCAGGCATTAGG |
| | | TTCCCCCTCACGTTTGGATGGTGTTTCAAGTTGGTACCGTT |
| | | GCTCATTAAAAAAGAAAAAATCTACCTGGCCTGGGTCCCGG |
| | | CGCACAAGGGTATAGGGGGGAACGAGCAAATTGATAAGCT |
| | | CGTGTCAAGGGCGAAGCGCGCGCCAGTCAAACAGACCTTG |
| | | AATTTCGACCTCCTTAAGCTCGCTGGAGACGTCGAATCCAA |
| | | CCCTGGCCCGATGCTGCCACAAATCACATTGTGGCAACGA |
| | | CCCATTGTAACAATAAAAATCGGGGGCCAGATCAAAGAAG |
| | | CGCTGCTTGACACCGGCGCCGACGATACAGTCCTCGAGGA |
| | | TATGAATTTGCCAGGCAAATGGAAGCCGAAGATGATTGGC |
| | | GGCATTGGCGGCTTTATTAAGGTTAAACAGTATGATCAGTG |
| | | GGGATTGACCACTCCCGATAAGAAGCATCAGAAAGATCCG |
| | | CCTTTTCTGTGGATGGGGTACGAACTGCACCCTGATCGATG |
| | | GACGGTCCAGCCGATAGAGCTGCCGGAAAAAGAATCATGG |
| | | ACCGTGAATGATATTCAAAAACTGATCGGAAAACTCAATTG |
| | | GGCGTCCCAGATATATGCTGGCATCAAAGTTGCAGCACAA |
| | | GAAGAGGAAGAGGTAGGTTTCCCGGTTCGGCCGCAAGTTC |
| | | CCTTGCGACCGATGACATACAAGGGCGCATTGGACCTTTCT |
| | | CACTTCCTCAAGGAAAAGGGCGGTTTGGAGGGCATCACTA |
| | | AACTTCAGAATTTCAGAGTCTACTATAGAGATAACAGGGAC |
| | | CCATTGTGGAAGGGCCCCGCTCGACTTCTCTGGAAAGGGG |
| | | AGGGAGCGGTTGTAATTCAAGACAACAGTGAAATTAAGGT |
| | | CGTCCCACGACGGAAGGTTAAAATAATTCGCGACTATGGC |
| | | AAGCGAATGGCGGGAGACGACTGTGTAGCAGGACGACAAG |
| | | ACGAGGACCCAAAGTTTAGATTGCCGATCCAGAAAGAGAC |
| | | ATGGGATACGTGGTGGACGGACTATTGGCAGGCCACCTGG |
| | | ATACCAGAATGGGAGTTTACAAACACTCCTCCACTCGTGAA |
| | | ATTGTGGTATCAACTTGAGACCGAGCCCATAGCTGGTGTAG |
| | | AGACGTTTTACGTTGACGGTGCTAGCAACAGGGAAACAAA |
| | | GGCCGCTGGAACCGTGCTCATCGGTCCTACTCCTGTGAAC |
| | | ATAATTGGACGAAATTTGTTGACCCAGCTGGGATGCACCCT |
| | | CAATTTCCCCATTAGCCCAATAGATACCGTACCAGTCAAGC |
| | | TTAAGCCTGGTATGGACGGTCCGCGAGTTAAGCAATGGCC |
| | | ACTTACTGAGGAGAAAATCAAGGCACTCATCGAGATCTGCA |
| | | CCGAAATGGAGAAGGAGGGCAAAATAAGCAGGATTGGTCC |
| | | CGAGAATCCATATAATACGCCGATCTTCGCGATAAAAAAGA |
| | | AGGACGGCACCAAATGGCGAAAACTGGTTGACTTCCGGGA |
| | | GCTTAACAAAAAAACTCAGGATTTTTGGGAAGTTCAACTCG |
| | | GGATCCCACACCCGTCTGGTCTTAAAAAAAAAAAAAGCGTA |
| | | ACAGTCCTTGACATCGGCGATGCCTACTTTAGCGTGCCTCT |
| | | CGATAAGGAGTTCAGAAAATACACGGCTTTCACTGTACCAA |
| | | GCACAAACAATGAGACTCCTGGGGTCAGATACCAGTACAA |
| | | TGTCCTTCCCATGGGGTGGAAAGGAAGCCCCGCAATATTC |
| | | CAGTGCTCAATGACGTGAtaggtttaaactcccatttaaatgtgagggttaatgcttcg |
| | | 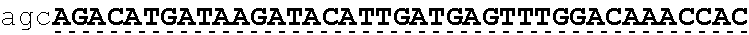 |
| | | 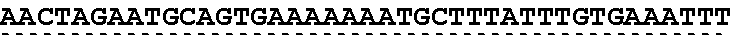 |
| | | 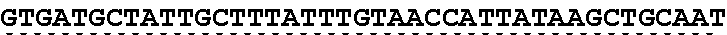 |
| | | 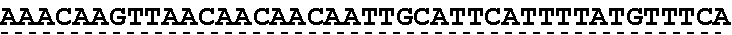 |
| | | 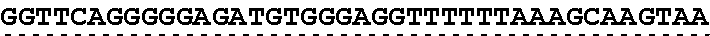 |
| | | 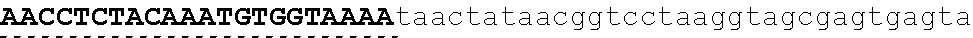 |
| | | gtgttctggggcgggggaggacctgcatgagggccagaataactgaaatctgtgcttttctgtgtgttgca |
| | | gcagcatgagcggaagcggctcctttgagggaggggtattcagcccttatctgacggggcgtctcccct |
| | | cctgggcgggagtgcgtcagaatgtgatgggatccacggtggacggccggcccgtgcagcccgcga |
| | | actcttcaaccctgacctatgcaaccctgagctcttcgtcgttggacgcagctgccgccgcagctgctgc |
| | | atctgccgccagcgccgtgcgcggaatggccatgggcgccggctactacggcactctggtggccaact |
| | | cgagttccaccaataatcccgccagcctgaacgaggagaagctgttgctgctgatggcccagctcgag |
| | | gccttgacccagcgcctgggcgagctgacccagcaggtggctcagctgcaggagcagacgcgggcc |
| | | gcggttgccacggtgaaatccaaataaaaaatgaatcaataaataaacggagacggttgttgattttaaca |
| | | cagagtctgaatctttatttgatttttcgcgcgcggtaggccctggaccaccggtctcgatcattgagcacc |
| | | cggtggatcttttccaggacccggtagaggtgggcttggatgttgaggtacatgggcatgagcccgtcc |
| | | cgggggtggaggtagctccattgcagggcctcgtgctcgggggtggtgttgtaaatcacccagtcatag |
| | | caggggcgcagggcatggtgttgcacaatatctttgaggaggagactgatggccacgggcagcccttt |
| | | ggtgtaggtgtttacaaatctgttgagctgggagggatgcatgcggggggagatgaggtgcatcttggc |
| | | ctggatcttgagattggcgatgttaccgcccagatcccgcctggggttcatgttgtgcaggaccaccagc |
| | | acggtgtatccggtgcacttggggaatttatcatgcaacttggaagggaaggcgtgaaagaatttggcga |
| | | cgcctttgtgcccgcccaggttttccatgcactcatccatgatgatggcgatgggcccgtgggcggcgg |
| | | cctgggcaaagacgtttcgggggtcggacacatcatagttgtggtcctgggtgaggtcatcataggccat |
| | | tttaatgaatttggggcggagggtgccggactgggggacaaaggtaccctcgatcccgggggcgtagt |
| | | tcccctcacagatctgcatctcccaggctttgagctcggagggggggatcatgtccacctgcggggcga |
| | | taaagaacacggtttccggggcgggggagatgagctgggccgaaagcaagttccggagcagctggg |
| | | acttgccgcagccggtggggccgtagatgaccccgatgaccggctgcaggtggtagttgagggagag |
| | | acagctgccgtcctcccggaggaggggggccacctcgttcatcatctcgcgcacgtgcatgttctcgcg |
| | | caccagttccgccaggaggcgctctccccccagggataggagctcctggagcgaggcgaagtttttca |
| | | gcggcttgagtccgtcggccatgggcattttggagagggtttgttgcaagagttccaggcggtcccaga |
| | | gctcggtgatgtgctctacggcatctcgatccagcagacctcctcgtttcgcgggttgggacggctgcgg |
| | | gagtagggcaccagacgatgggcgtccagcgcagccagggtccggtccttccagggtcgcagegtcc |
| | | gcgtcagggtggtctccgtcacggtgaaggggtgcgcgccgggctgggcgcttgcgagggtgcgctt |
| | | caggctcatccggctggtcgaaaaccgctcccgatcggcgccctgcgcgtcggccaggtagcaattga |
| | | ccatgagttcgtagttgagcgcctcggccgcgtggcctttggcgcggagcttacctttggaagtctgccc |
| | | gcaggcgggacagaggagggacttgagggcgtagagcttgggggcgaggaagacggactcgggg |
| | | gcgtaggcgtccgcgccgcagtgggcgcagacggtctcgcactccacgagccaggtgaggtcgggc |
| | | tggtcggggtcaaaaaccagtttcccgccgttctttttgatgcgtttcttacctttggtctccatgagctcgtgt |
| | | ccccgctgggtgacaaagaggctgtccgtgtccccgtagaccgactttatgggccggtcctcgagcggt |
| | | gtgccgcggtcctcctcgtagaggaaccccgcccactccgagacgaaagcccgggtccaggccagca |
| | | cgaaggaggccacgtgggacgggtagcggtcgttgtccaccagcgggtccaccttttccagggtatgc |
| | | aaacacatgtccccctcgtccacatccaggaaggtgattggcttgtaagtgtaggccacgtgaccgggg |
| | | gtcccggccgggggggtataaaagggtgcgggtccctgctcgtcctcactgtcttccggatcgctgtcc |
| | | aggagcgccagctgttggggtaggtattccctctcgaaggcgggcatgacctcggcactcaggttgtca |
| | | gtttctagaaacgaggaggatttgatattgacggtgccggcggagatgcctttcaagagcccctcgtcca |
| | | tctggtcagaaaagacgatctttttgttgtcgagcttggtggcgaaggagccgtagagggcgttggagag |
| | | gagcttggcgatggagcgcatggtctggtttttttccttgtcggcgcgctccttggcggcgatgttgagctg |
| | | cacgtactcgcgcgccacgcacttccattcggggaagacggtggtcagctcgtcgggcacgattctgac |
| | | ctgccagccccgattatgcagggtgatgaggtccacactggtggccacctcgccgcgcaggggctcat |
| | | tagtccagcagaggcgtccgcccttgcgcgagcagaaggggggcagggggtccagcatgacctcgt |
| | | cgggggggtcggcatcgatggtgaagatgccgggcaggaggtcggggtcaaagtagctgatggaag |
| | | tggccagatcgtccagggcagcttgccattcgcgcacggccagcgcgcgctcgtagggactgagggg |
| | | cgtgccccagggcatgggatgggtaagcgcggaggcgtacatgccgcagatgtcgtagacgtagagg |
| | | ggctcctcgaggatgccgatgtaggtggggtagcagcgccccccgcggatgctggcgcgcacgtagt |
| | | catacagctcgtgcgagggggcgaggagccccgggcccaggttggtgcgactgggcttttcggcgcg |
| | | gtagacgatctggcggaaaatggcatgcgagttggaggagatggtgggcctttggaagatgttgaagtg |
| | | ggcgtggggcagtccgaccgagtcgcggatgaagtgggcgtaggagtcttgcagcttggcgacgagc |
| | | tcggcggtgactaggacgtccagagcgcagtagtcgagggtctcctggatgatgtcatacttgagctgtc |
| | | ccttttgtttccacagctcgcggttgagaaggaactcttcgcggtccttccagtactcttcgagggggaac |
| | | ccgtcctgatctgcacggtaagagcctagcatgtagaactggttgacggccttgtaggcgcagcagccc |
| | | ttctccacggggagggcgtaggcctgggcggccttgcgcagggaggtgtgcgtgagggcgaaagtgt |
| | | ccctgaccatgaccttgaggaactggtgcttgaagtcgatatcgtcgcagcccccctgctcccagagctg |
| | | gaagtccgtgcgcttcttgtaggcggggttgggcaaagcgaaagtaacatcgttgaagaggatcttgcc |
| | | cgcgcggggcataaagttgcgagtgatgcggaaaggttggggcacctcggcccggttgttgatgacct |
| | | gggcggcgagcacgatctcgtcgaagccgttgatgttgtggcccacgatgtagagttccacgaatcgcg |
| | | gacggcccttgacgtggggcagtttcttgagctcctcgtaggtgagctcgtcggggtcgctgagcccgt |
| | | gctgctcgagcgcccagtcggcgagatgggggttggcgcggaggaaggaagtccagagatccacgg |
| | | ccagggcggtttgcagacggtcccggtactgacggaactgctgcccgacggccattttttcgggggtga |
| | | cgcagtagaaggtgcgggggtccccgtgccagcgatcccatttgagctggagggcgagatcgagggc |
| | | gagctcgacgagccggtcgtccccggagagtttcatgaccagcatgaaggggacgagctgcttgccg |
| | | aaggaccccatccaggtgtaggtttccacatcgtaggtgaggaagagcctttcggtgcgaggatgcgag |
| | | ccgatggggaagaactggatctcctgccaccaattggaggaatggctgttgatgtgatggaagtagaaa |
| | | tgccgacggcgcgccgaacactcgtgcttgtgtttatacaagcggccacagtgctcgcaacgctgcacg |
| | | ggatgcacgtgctgcacgagctgtacctgagttcctttgacgaggaatttcagtgggaagtggagtcgtg |
| | | gcgcctgcatctcgtgctgtactacgtcgtggtggtcggcctggccctcttctgcctcgatggtggtcatg |
| | | ctgacgagcccgcgcgggaggcaggtccagacctcggcgcgagcgggtcggagagcgaggacga |
| | | gggcgcgcaggccggagctgtccagggtcctgagacgctgcggagtcaggtcagtgggcagcggc |
| | | ggcgcgcggttgacttgcaggagtttttccagggcgcgcgggaggtccagatggtacttgatctccacc |
| | | gcgccattggtggcgacgtcgatggcttgcagggtcccgtgcccctggggtgtgaccaccgtcccccg |
| | | tttcttcttgggcggctggggcgacgggggcggtgcctcttccatggttagaagcggcggcgaggacg |
| | | cgcgccgggcggcaggggcggctcggggcccggaggcaggggcggcaggggcacgtcggcgcc |
| | | gcgcgcgggtaggttctggtactgcgcccggagaagactggcgtgagcgacgacgcgacggttgac |
| | | gtcctggatctgacgcctctgggtgaaggccacgggacccgtgagtttgaacctgaaagagagttcgac |
| | | agaatcaatctcggtatcgttgacggcggcctgccgcaggatctcttgcacgtcgcccgagttgtcctgg |
| | | taggcgatctcggtcatgaactgctcgatctcctcctcttgaaggtctccgcggccggcgcgctccacgg |
| | | tggccgcgaggtcgttggagatgcggcccatgagctgcgagaaggcgttcatgcccgcctcgttccag |
| | | acgcggctgtagaccacgacgccctcgggatcgcgggcgcgcatgaccacctgggcgaggttgagct |
| | | ccacgtggcgcgtgaagaccgcgtagttgcagaggcgctggtagaggtagttgagcgtggtggcgat |
| | | gtgctcggtgacgaagaaatacatgatccagcggcggagcggcatctcgctgacgtcgcccagcgcct |
| | | ccaaacgttccatggcctcgtaaaagtccacggcgaagttgaaaaactgggagttgcgcgccgagacg |
| | | gtcaactcctcctccagaagacggatgagctcggcgatggtggcgcgcacctegcgctcgaaggccc |
| | | ccgggagttcctccacttcctcttcttcctcctccactaacatctcttctacttcctcctcaggcggcagtggt |
| | | ggcgggggagggggcctgcgtcgccggcggcgcacgggcagacggtcgatgaagcgctcgatggt |
| | | ctcgccgcgccggcgtcgcatggtctcggtgacggcgcgcccgtcctcgcggggccgcagcgtgaa |
| | | gacgccgccgcgcatctccaggtggccgggggggtccccgttgggcagggagagggcgctgacgat |
| | | gcatcttatcaattgccccgtagggactccgcgcaaggacctgagcgtctcgagatccacgggatctga |
| | | aaaccgctgaacgaaggcttcgagccagtcgcagtcgcaaggtaggctgagcacggtttcttctggcg |
| | | ggtcatgttggttgggagcggggcgggcgatgctgctggtgatgaagttgaaataggcggttctgagac |
| | | ggcggatggtggcgaggagcaccaggtctttgggcccggcttgctggatgcgcagacggtcggccat |
| | | gccccaggcgtggtcctgacacctggccaggtccttgtagtagtcctgcatgagccgctccacgggcac |
| | | ctcctcctcgcccgcgcggccgtgcatgcgcgtgagcccgaagccgcgctggggctggacgagcgc |
| | | caggtcggcgacgacgcgctcggcgaggatggcttgctggatctgggtgagggtggtctggaagtcat |
| | | caaagtcgacgaagcggtggtaggctccggtgttgatggtgtaggagcagttggccatgacggaccag |
| | | ttgacggtctggtggcccggacgcacgagctcgtggtacttgaggcgcgagtaggcgcgcgtgtcgaa |
| | | gatgtagtcgttgcaggtgcgcaccaggtactggtagccgatgaggaagtgcggcggcggctggcgg |
| | | tagagcggccatcgctcggtggcgggggcgccgggcgcgaggtcctcgagcatggtgcggtggtag |
| | | ccgtagatgtacctggacatccaggtgatgccggcggcggtggtggaggcgcgcgggaactcgcgg |
| | | acgcggttccagatgttgcgcagcggcaggaagtagttcatggtgggcacggtctggcccgtgaggcg |
| | | cgcgcagtcgtggatgctctatacgggcaaaaacgaaagcggtcagcggctcgactccgtggcctgga |
| | | ggctaagcgaacgggttgggctgcgcgtgtaccccggttcgaatctcgaatcaggctggagccgcagc |
| | | taacgtggtattggcactcccgtctcgacccaagcctgcaccaaccctccaggatacggaggcgggtc |
| | | gttttgcaacttttttttggaggccggatgagactagtaagcgcggaaagcggccgaccgcgatggctcg |
| | | ctgccgtagtctggagaagaatcgccagggttgcgttgcggtgtgccccggttcgaggccggccggatt |
| | | ccgcggctaacgagggcgtggctgccccgtcgtttccaagaccccatagccagccgacttctccagtta |
| | | cggagcgagcccctcttttgttttgtttgtttttgccagatgcatcccgtactgcggcagatgegcccccac |
| | | caccctccaccgcaacaacagccccctccacagccggcgcttctgcccccgccccagcagcaacttcc |
| | | agccacgaccgccgcggccgccgtgagcggggctggacagagttatgatcaccagctggccttggaa |
| | | gagggcgaggggctggcgcgcctgggggcgtcgtcgccggagcggcacccgcgcgtgcagatgaa |
| | | aagggacgctcgcgaggcctacgtgcccaagcagaacctgttcagagacaggagcggcgaggagcc |
| | | cgaggagatgcgcgcggcccggttccacgcggggcgggagctgcggcgcggcctggaccgaaag |
| | | agggtgctgagggacgaggatttcgaggcggacgagctgacggggatcagccccgcgcgcgcgca |
| | | cgtggccgcggccaacctggtcacggcgtacgagcagaccgtgaaggaggagagcaacttccaaaa |
| | | atccttcaacaaccacgtgcgcaccctgatcgcgcgcgaggaggtgaccctgggcctgatgcacctgt |
| | | gggacctgctggaggccatcgtgcagaaccccaccagcaagccgctgacggcgcagctgttcctggt |
| | | ggtgcagcatagtcgggacaacgaagcgttcagggaggcgctgctgaatatcaccgagcccgagggc |
| | | cgctggctcctggacctggtgaacattctgcagagcatcgtggtgcaggagcgcgggctgccgctgtc |
| | | cgagaagctggcggccatcaacttctcggtgctgagtttgggcaagtactacgctaggaagatctacaa |
| | | gaccccgtacgtgcccatagacaaggaggtgaagatcgacgggttttacatgcgcatgaccctgaaagt |
| | | gctgaccctgagcgacgatctgggggtgtaccgcaacgacaggatgcaccgtgcggtgagcgccagc |
| | | aggcggcgcgagctgagcgaccaggagctgatgcatagtctgcagcgggccctgaccggggccgg |
| | | gaccgagggggagagctactttgacatgggcgcggacctgcactggcagcccagccgccgggcctt |
| | | ggaggcggcggcaggaccctacgtagaagaggtggacgatgaggtggacgaggagggcgagtacc |
| | | tggaagactgatggcgcgaccgtatttttgctagatgcaacaacaacagccacctcctgatcccgcgatg |
| | | cgggcggcgctgcagagccagccgtccggcattaactcctcggacgattggacccaggccatgcaac |
| | | gcatcatggcgctgacgacccgcaaccccgaagcctttagacagcagccccaggccaaccggetctc |
| | | ggccatcctggaggccgtggtgccctcgcgctccaaccccacgcacgagaaggtcctggccatcgtg |
| | | aacgcgctggtggagaacaaggccatccgcggcgacgaggccggcctggtgtacaacgcgctgctg |
| | | gagcgcgtggcccgctacaacagcaccaacgtgcagaccaacctggaccgcatggtgaccgacgtgc |
| | | gcgaggccgtggcccagcgcgagcggttccaccgcgagtccaacctgggatccatggtggcgctgaa |
| | | cgccttcctcagcacccagcccgccaacgtgccccggggccaggaggactacaccaacttcatcagc |
| | | gccctgcgcctgatggtgaccgaggtgccccagagcgaggtgtaccagtccgggccggactacttctt |
| | | ccagaccagtcgccagggcttgcagaccgtgaacctgagccaggctttcaagaacttgcagggcctgt |
| | | ggggcgtgcaggccccggtcggggaccgcgcgacggtgtcgagcctgctgacgccgaactcgcgc |
| | | ctgctgctgctgctggtggcccccttcacggacageggcagcatcaaccgcaactcgtacctgggctac |
| | | ctgattaacctgtaccgcgaggccatcggccaggcgcacgtggacgagcagacctaccaggagatca |
| | | cccacgtgagccgcgccctgggccaggacgacccgggcaacctggaagccaccctgaactttttgctg |
| | | accaaccggtcgcagaagatcccgccccagtacgcgctcagcaccgaggaggagcgcatcctgcgtt |
| | | acgtgcagcagagcgtgggcctgttcctgatgcaggagggggccacccccagcgccgcgctcgacat |
| | | gaccgcgcgcaacatggagcccagcatgtacgccagcaaccgcccgttcatcaataaactgatggact |
| | | acttgcatcgggcggccgccatgaactctgactatttcaccaacgccatcctgaatccccactggetccc |
| | | gccgccggggttctacacgggcgagtacgacatgcccgaccccaatgacgggttcctgtgggacgat |
| | | gtggacagcagcgtgttctccccccgaccgggtgctaacgagcgccccttgtggaagaaggaaggca |
| | | gcgaccgacgcccgtcctcggcgctgtccggccgcgagggtgctgccgcggcggtgcccgaggcc |
| | | gccagtcctttcccgagcttgcccttctcgctgaacagtatccgcagcagcgagctgggcaggatcacg |
| | | cgcccgcgcttgctgggcgaagaggagtacttgaatgactcgctgttgagacccgagcgggagaaga |
| | | acttccccaataacgggatagaaagcctggtggacaagatgagccgctggaagacgtatgcgcagga |
| | | gcacagggacgatccccgggcgtcgcagggggccacgagccggggcagcgccgcccgtaaacgc |
| | | cggtggcacgacaggcagcggggacagatgtgggacgatgaggactccgccgacgacagcagcgt |
| | | gttggacttggggggagtggtaacccgttcgctcacctgcgcccccgtatcgggcgcatgatgtaaga |
| | | gaaaccgaaaataaatgatactcaccaaggccatggcgaccagcgtgcgttcgtttcttctctgttgttgtt |
| | | gtatctagtatgatgaggcgtgcgtacccggagggtcctcctccctcgtacgagagcgtgatgcagcag |
| | | gcgatggcggcggcggcgatgcagcccccgctggaggctccttacgtgcccccgcggtacctggcg |
| | | cctacggaggggcggaacagcattcgttactcggagctggcacccttgtacgataccacccggttgtac |
| | | ctggtggacaacaagtcggcggacatcgcctcgctgaactaccagaacgaccacagcaacttcctgac |
| | | caccgtggtgcagaacaatgacttcacccccacggaggccagcacccagaccatcaactttgacgagc |
| | | gctcgcggtggggcggccagctgaaaaccatcatgcacaccaacatgcccaacgtgaacgagttcatg |
| | | tacagcaacaagttcaaggcgcgggtgatggtctcccgcaagacccccaatggggtgacagtgacag |
| | | aggattatgatggtagtcaggatgagctgaagtatgaatgggtggaatttgagctgcccgaaggcaactt |
| | | ctcggtgaccatgaccatcgacctgatgaacaacgccatcatcgacaattacttggcggtggggcggca |
| | | gaacggggtgctggagagcgacatcggcgtgaagttcgacactaggaacttcaggctgggctgggac |
| | | cccgtgaccgagctggtcatgcccggggtgtacaccaacgaggctttccatcccgatattgtcttgctgc |
| | | ccggctgcggggtggacttcaccgagagccgcctcagcaacctgctgggcattcgcaagaggcagcc |
| | | cttccaggaaggcttccagatcatgtacgaggatctggaggggggcaacatccccgcgctcctggatgt |
| | | cgacgcctatgagaaaagcaaggaggatgcagcagctgaagcaactgcagccgtagctaccgcctct |
| | | accgaggtcaggggcgataattttgcaagcgccgcagcagtggcagcggccgaggcggctgaaacc |
| | | gaaagtaagatagtcattcagccggtggagaaggatagcaagaacaggagctacaacgtactaccgga |
| | | caagataaacaccgcctaccgcagctggtacctagcctacaactatggcgaccccgagaagggcgtgc |
| | | gctcctggacgctgctcaccacctcggacgtcacctgcggcgtggagcaagtctactggtcgctgcccg |
| | | acatgatgcaagacccggtcaccttccgctccacgcgtcaagttagcaactacccggtggtgggcgcc |
| | | gagctcctgcccgtctactccaagagcttcttcaacgagcaggccgtctactcgcagcagctgcgcgcct |
| | | tcacctcgcttacgcacgtcttcaaccgcttccccgagaaccagatcctcgtccgcccgcccgcgccca |
| | | ccattaccaccgtcagtgaaaacgttcctgctctcacagatcacgggaccctgccgctgcgcagcagtat |
| | | ccggggagtccagcgcgtgaccgttactgacgccagacgccgcacctgcccctacgtctacaaggcc |
| | | ctgggcatagtcgcgccgcgcgtcctctcgagccgcaccttctaaatgtccattctcatctcgcccagtaa |
| | | taacaccggttggggcctgcgcgcgcccagcaagatgtacggaggcgctcgccaacgctccacgcaa |
| | | caccccgtgcgcgtgcgcgggcacttccgcgctccctggggcgccctcaagggccgcgtgcggtcgc |
| | | gcaccaccgtcgacgacgtgatcgaccaggtggtggccgacgcgcgcaactacacccccgccgccg |
| | | cgcccgtctccaccgtggacgccgtcatcgacagcgtggtggccgacgcgcgccggtacgcccgcg |
| | | ccaagagccggcggcggcgcatcgcccggcggcaccggagcacccccgccatgcgcgcggcgcg |
| | | agccttgctgcgcagggccaggcgcacgggacgcagggccatgctcagggcggccagacgcgcgg |
| | | cttcaggcgccagcgccggcaggacccggagacgcgcggccacggcggcggcagcggccatcgc |
| | | cagcatgtcccgcccgcggcgagggaacgtgtactgggtgcgcgacgccgccaccggtgtgcgcgt |
| | | gcccgtgcgcacccgcccccctcgcacttgaagatgttcacttcgcgatgttgatgtgtcccagcggcga |
| | | ggaggatgtccaagcgcaaattcaaggaagagatgctccaggtcatcgcgcctgagatctacggccct |
| | | gcggtggtgaaggaggaaagaaagccccgcaaaatcaagcgggtcaaaaaggacaaaaaggaaga |
| | | agaaagtgatgtggacggattggtggagtttgtgcgcgagttcgccccccggcggcgcgtgcagtggc |
| | | gcgggcggaaggtgcaaccggtgctgagacccggcaccaccgtggtcttcacgcccggcgagcgct |
| | | ccggcaccgcttccaagcgctcctacgacgaggtgtacggggatgatgatattctggagcaggcggcc |
| | | gagcgcctgggcgagtttgcttacggcaagcgcagccgttccgcaccgaaggaagaggcggtgtcca |
| | | tcccgctggaccacggcaaccccacgccgagcctcaagcccgtgaccttgcagcaggtgctgccgac |
| | | cgcggcgccgcgccgggggttcaagcgcgagggcgaggatctgtaccccaccatgcagctgatggt |
| | | gcccaagcgccagaagctggaagacgtgctggagaccatgaaggtggacccggacgtgcagcccga |
| | | ggtcaaggtgcggcccatcaagcaggtggccccgggcctgggcgtgcagaccgtggacatcaagatt |
| | | cccacggagcccatggaaacgcagaccgagcccatgatcaagcccagcaccagcaccatggaggtg |
| | | cagacggatccctggatgccatcggctcctagtcgaagaccccggcgcaagtacggcgcggccagcc |
| | | tgctgatgcccaactacgcgctgcatccttccatcatccccacgccgggctaccgcggcacgcgcttcta |
| | | ccgcggtcataccagcagccgccgccgcaagaccaccactcgccgccgccgtcgccgcaccgccgc |
| | | tgcaaccacccctgccgccctggtgcggagagtgtaccgccgcggccgcgcacctctgaccctgccg |
| | | cgcgcgcgctaccacccgagcatcgccatttaaactttcgcctgctttgcagatcaatggccctcacatgc |
| | | cgccttcgcgttcccattacgggctaccgaggaagaaaaccgcgccgtagaaggctggcggggaacg |
| | | ggatgcgtcgccaccaccaccggcggcggcgcgccatcagcaagcggttggggggaggcttcctgc |
| | | ccgcgctgatccccatcatcgccgcggcgatcggggcgatccccggcattgcttccgtggcggtgcag |
| | | gcctctcagcgccactgagacacacttggaaacatcttgtaataaaccaatggactctgacgctcctggtc |
| | | ctgtgatgtgttttcgtagacagatggaagacatcaatttttcgtccctggctccgcgacacggcacgcgg |
| | | ccgttcatgggcacctggagcgacatcggcaccagccaactgaacgggggcgccttcaattggagca |
| | | gtctctggagcgggcttaagaatttcgggtccacgcttaaaacctatggcagcaaggcgtggaacagca |
| | | ccacagggcaggcgctgagggataagctgaaagagcagaacttccagcagaaggtggtcgatgggct |
| | | cgcctcgggcatcaacggggtggtggacctggccaaccaggccgtgcagcggcagatcaacagccg |
| | | cctggacccggtgccgcccgccggctccgtggagatgccgcaggtggaggaggagctgcctcccct |
| | | ggacaagcggggcgagaagcgaccccgccccgatgcggaggagacgctgctgacgcacacggac |
| | | gagccgcccccgtacgaggaggcggtgaaactgggtctgcccaccacgcggcccategcgcccctg |
| | | gccaccggggtgctgaaacccgaaaagcccgcgaccctggacttgcctcctccccagccttccegccc |
| | | ctctacagtggctaagcccctgccgccggtggccgtggcccgcgcgcgacccgggggcaccgcccg |
| | | ccctcatgcgaactggcagagcactctgaacagcatcgtgggtctgggagtgcagagtgtgaagcgcc |
| | | gccgctgctattaaacctaccgtagcgcttaacttgcttgtctgtgtgtgtatgtattatgtcgccgccgccg |
| | | ctgtccaccagaaggaggagtgaagaggcgcgtcgccgagttgcaagatggccaccccatcgatgct |
| | | gccccagtgggcgtacatgcacatcgccggacaggacgcttcggagtacctgagtccgggtctggtgc |
| | | agtttgcccgcgccacagacacctacttcagtctggggaacaagtttaggaaccccacggtggcgccca |
| | | cgcacgatgtgaccaccgaccgcagccagcggctgacgctgcgcttcgtgcccgtggaccgcgagg |
| | | acaacacctactcgtacaaagtgcgctacacgctggccgtgggcgacaaccgcgtgctggacatggcc |
| | | agcacctactttgacatccgcggcgtgctggatcggggccctagcttcaaaccctactceggcaccgcct |
| | | acaacagtctggcccccaagggagcacccaacacttgtcagtggacatataaagccgatggtgaaact |
| | | gccacagaaaaaacctatacatatggaaatgcacccgtgcagggcattaacatcacaaaagatggtattc |
| | | aacttggaactgacaccgatgatcagccaatctacgcagataaaacctatcagcctgaacctcaagtggg |
| | | tgatgctgaatggcatgacatcactggtactgatgaaaagtatggaggcagagctcttaagcctgatacc |
| | | aaaatgaagccttgttatggttcttttgccaagcctactaataaagaaggaggtcaggcaaatgtgaaaac |
| | | aggaacaggcactactaaagaatatgacatagacatggctttctttgacaacagaagtgcggctgctgct |
| | | ggcctagctccagaaattgttttgtatactgaaaatgtggatttggaaactccagatacccatattgtataca |
| | | aagcaggcacagatgacagcagctcttctattaatttgggtcagcaagccatgcccaacagacctaacta |
| | | cattggtttcagagacaactttatcgggctcatgtactacaacagcactggcaatatgggggtgctggccg |
| | | gtcaggcttctcagctgaatgctgtggttgacttgcaagacagaaacaccgagctgtcctaccagctcttg |
| | | cttgactctctgggtgacagaacccggtatttcagtatgtggaatcaggcggtggacagctatgatcctga |
| | | tgtgcgcattattgaaaatcatggtgtggaggatgaacttcccaactattgtttccctctggatgctgttggc |
| | | agaacagatacttatcagggaattaaggctaatggaactgatcaaaccacatggaccaaagatgacagt |
| | | gtcaatgatgctaatgagataggcaagggtaatccattcgccatggaaatcaacatccaagccaacctgt |
| | | ggaggaacttcctctacgccaacgtggccctgtacctgcccgactcttacaagtacacgccggccaatgt |
| | | taccctgcccaccaacaccaacacctacgattacatgaacggccgggtggtggcgccctcgctggtgg |
| | | actcctacatcaacatcggggcgcgctggtcgctggatcccatggacaacgtgaaccccttcaaccacc |
| | | accgcaatgcggggctgcgctaccgctccatgctcctgggcaacgggcgctacgtgcccttccacatc |
| | | caggtgccccagaaatttttcgccatcaagagcctcctgctcctgcccgggtcctacacctacgagtgga |
| | | acttccgcaaggacgtcaacatgatcctgcagagctccctcggcaacgacctgcgcacggacggggc |
| | | ctccatctccttcaccagcatcaacctctacgccaccttcttccccatggcgcacaacacggcctccacgc |
| | | tcgaggccatgctgcgcaacgacaccaacgaccagtccttcaacgactacctctcggcggccaacatg |
| | | ctctaccccatcccggccaacgccaccaacgtgcccatctccatcccctegcgcaactgggccgccttc |
| | | cgcggctggtccttcacgcgtctcaagaccaaggagacgccctcgctgggctccgggttcgaccccta |
| | | cttcgtctactegggctccatcccctacctcgacggcaccttctacctcaaccacaccttcaagaaggtctc |
| | | catcaccttcgactcctccgtcagctggcccggcaacgaccggctcctgacgcccaacgagttcgaaat |
| | | caagcgcaccgtcgacggcgagggctacaacgtggcccagtgcaacatgaccaaggactggttcctg |
| | | gtccagatgctggcccactacaacatcggctaccagggcttctacgtgcccgagggctacaaggaccg |
| | | catgtactccttcttccgcaacttccagcccatgagccgccaggtggtggacgaggtcaactacaaggac |
| | | taccaggccgtcaccctggcctaccagcacaacaactegggcttcgtcggctacctcgcgcccaccatg |
| | | cgccagggccagccctaccccgccaactacccctacccgctcatcggcaagagcgccgtcaccagcg |
| | | tcacccagaaaaagttcctctgcgacagggtcatgtggcgcatccccttctccagcaacttcatgtccatg |
| | | ggcgcgctcaccgacctcggccagaacatgctctatgccaactccgcccacgcgctagacatgaatttc |
| | | gaagtcgaccccatggatgagtccacccttctctatgttgtcttcgaagtcttcgacgtcgtccgagtgcac |
| | | cagccccaccgcggcgtcatcgaggccgtctacctgcgcacccccttctcggccggtaacgccaccac |
| | | ctaagctcttgcttcttgcaagccatggccgcgggctccggcgagcaggagctcagggccatcatccgc |
| | | gacctgggctgcgggccctacttcctgggcaccttcgataagcgcttcccgggattcatggccccgcac |
| | | aagctggcctgcgccatcgtcaacacggccggccgcgagaccgggggcgagcactggctggccttc |
| | | gcctggaacccgcgctcgaacacctgctacctcttcgaccccttcgggttctcggacgagcgcctcaag |
| | | cagatctaccagttcgagtacgagggcctgctgcgccgcagcgccctggccaccgaggaccgctgcg |
| | | tcaccctggaaaagtccacccagaccgtgcagggtccgcgctcggccgcctgcgggctcttctgctgc |
| | | atgttcctgcacgccttcgtgcactggcccgaccgccccatggacaagaaccccaccatgaacttgctg |
| | | acgggggtgcccaacggcatgctccagtcgccccaggtggaacccaccctgcgccgcaaccaggag |
| | | gcgctctaccgcttcctcaactcccactccgcctactttcgctcccaccgegcgcgcatcgagaaggcca |
| | | ccgccttcgaccgcatgaatcaagacatgtaaaccgtgtgtgtatgttaaatgtctttaataaacagcacttt |
| | | catgttacacatgcatctgagatgatttatttagaaatcgaaagggttctgccgggtctcggcatggcccg |
| | | cgggcagggacacgttgcggaactggtacttggccagccacttgaactcggggatcagcagtttgggc |
| | | agcggggtgtcggggaaggagtcggtccacagcttccgcgtcagttgcagggcgcccagcaggtcg |
| | | ggcgcggagatcttgaaatcgcagttgggacccgcgttctgcgcgcgggagttgcggtacacggggtt |
| | | gcagcactggaacaccatcagggccgggtgcttcacgctcgccagcaccgtcgcgtcggtgatgctct |
| | | ccacgtcgaggtcctcggcgttggccatcccgaagggggtcatcttgcaggtctgccttcccatggtgg |
| | | gcacgcacccgggcttgtggttgcaatcgcagtgcagggggatcagcatcatctgggcctggtcggcg |
| | | ttcatccccgggtacatggccttcatgaaagcctccaattgcctgaacgcctgctgggccttggctccctc |
| | | ggtgaagaagaccccgcaggacttgctagagaactggttggtggcgcacccggcgtcgtgcacgcag |
| | | cagcgcgcgtcgttgttggccagctgcaccacgctgcgcccccagcggttctgggtgatcttggcccgg |
| | | tcggggttctccttcagcgcgcgctgcccgttctcgctcgccacatccatctcgatcatgtgctccttctgg |
| | | atcatggtggtcccgtgcaggcaccgcagcttgccctcggcctcggtgcacccgtgcagccacagcgc |
| | | gcacccggtgcactcccagttcttgtgggcgatctgggaatgcgcgtgcacgaagccctgcaggaagc |
| | | ggcccatcatggtggtcagggtcttgttgctagtgaaggtcagcggaatgccgcggtgctcctcgttgat |
| | | gtacaggtggcagatgcggcggtacacctcgccctgctcgggcatcagctggaagttggctttcaggtc |
| | | ggtctccacgcggtagcggtccatcagcatagtcatgatttccatacccttctcccaggccgagacgatg |
| | | ggcaggctcatagggttcttcaccatcatcttagcgctagcagccgcggccagggggtcgctctcgtcc |
| | | agggtctcaaagctccgcttgccgtccttctcggtgatccgcaccggggggtagctgaagcccacggc |
| | | cgccagctcctcctcggcctgtctttcgtcctcgctgtcctggctgacgtcctgcaggaccacatgcttggt |
| | | cttgcggggtttcttcttgggcggcagcggcggcggagatgttggagatggcgagggggagcgcgag |
| | | ttctcgctcaccactactatctcttcctcttcttggtccgaggccacgcggcggtaggtatgtctcttcgggg |
| | | gcagaggcggaggcgacgggctctcgccgccgcgacttggcggatggctggcagagccccttccgc |
| | | gttcgggggtgcgctcccggcggcgctctgactgacttcctccgcggccggccattgtgttctcctaggg |
| | | aggaacaacaagcatggagactcagccatcgccaacctcgccatctgcccccaccgccgacgagaag |
| | | cagcagcagcagaatgaaagcttaaccgccccgccgcccagccccgccacctccgacgeggccgtc |
| | | ccagacatgcaagagatggaggaatccatcgagattgacctgggctatgtgacgcccgcggagcacg |
| | | aggaggagctggcagtgcgcttttcacaagaagagatacaccaagaacagccagagcaggaagcag |
| | | agaatgagcagagtcaggctgggctcgagcatgacggcgactacctccacctgagcgggggggagg |
| | | acgcgctcatcaagcatctggcccggcaggccaccatcgtcaaggatgcgctgctcgaccgcaccga |
| | | ggtgcccctcagcgtggaggagctcagccgcgcctacgagttgaacctcttctcgcegcgcgtgcccc |
| | | ccaagcgccagcccaatggcacctgcgagcccaacccgcgcctcaacttctacccggtcttcgcggtg |
| | | cccgaggccctggccacctaccacatctttttcaagaaccaaaagatccccgtctcctgcegcgccaacc |
| | | gcacccgcgccgacgcccttttcaacctgggtcccggcgcccgcctacctgatatcgcctccttggaag |
| | | aggttcccaagatcttcgagggtctgggcagcgacgagactcgggccgcgaacgctctgcaaggaga |
| | | aggaggagagcatgagcaccacagcgccctggtcgagttggaaggcgacaacgcgcggctggcggt |
| | | gctcaaacgcacggtcgagctgacccatttcgcctacccggctctgaacctgccccccaaagtcatgag |
| | | cgcggtcatggaccaggtgctcatcaagcgcgcgtcgcccatctccgaggacgagggcatgcaagac |
| | | tccgaggagggcaagcccgtggtcagcgacgagcagctggcccggtggctgggtcctaatgctagtc |
| | | cccagagtttggaagagcggcgcaaactcatgatggccgtggtcctggtgaccgtggagctggagtgc |
| | | ctgcgccgcttcttcgccgacgcggagaccctgcgcaaggtcgaggagaacctgcactacctcttcag |
| | | gcacgggttcgtgcgccaggcctgcaagatctccaacgtggagctgaccaacctggtctcctacatggg |
| | | catcttgcacgagaaccgcctggggcagaacgtgctgcacaccaccctgcgcggggaggcccggcg |
| | | cgactacatccgcgactgcgtctacctctacctctgccacacctggcagacgggcatgggcgtgtggca |
| | | gcagtgtctggaggagcagaacctgaaagagctctgcaagctcctgcagaagaacctcaagggtctgt |
| | | ggaccgggttcgacgagcgcaccaccgcctcggacctggccgacctcattttccccgagcgcctcagg |
| | | ctgacgctgcgcaacggcctgcccgactttatgagccaaagcatgttgcaaaactttcgctctttcatcctc |
| | | gaacgctccggaatcctgcccgccacctgctccgcgctgccctcggacttcgtgccgctgaccttccgc |
| | | gagtgccccccgccgctgtggagccactgctacctgctgcgcctggccaactacctggcctaccactcg |
| | | gacgtgatcgaggacgtcagcggcgagggcctgctcgagtgccactgccgctgcaacctctgcacgc |
| | | cgcaccgctccctggcctgcaacccccagctgctgagcgagacccagatcatcggcaccttcgagttg |
| | | caagggcccagcgaaggcgagggttcagccgccaaggggggtctgaaactcaccccggggctgtgg |
| | | acctcggcctacttgcgcaagttcgtgcccgaggactaccatcccttcgagatcaggttctacgaggacc |
| | | aatcccatccgcccaaggccgagctgtcggcctgcgtcatcacccagggggcgatcctggcccaattg |
| | | caagccatccagaaatcccgccaagaattcttgctgaaaaagggccgcggggtctacctcgaccccca |
| | | gaccggtgaggagctcaaccccggcttcccccaggatgccccgaggaaacaagaagctgaaagtgg |
| | | agctgccgcccgtggaggatttggaggaagactgggagaacagcagtcaggcagaggaggaggag |
| | | atggaggaagactgggacagcactcaggcagaggaggacagcctgcaagacagtctggaggaaga |
| | | cgaggaggaggcagaggaggaggtggaagaagcagccgccgccagaccgtcgtcctcggcgggg |
| | | gagaaagcaagcagcacggataccatctccgctccgggtcggggtcccgctcgaccacacagtagat |
| | | gggacgagaccggacgattcccgaaccccaccacccagaccggtaagaaggagcggcagggatac |
| | | aagtcctggcgggggcacaaaaacgccatcgtctcctgcttgcaggcctgcgggggcaacatctccttc |
| | | acccggcgctacctgctcttccaccgcggggtgaactttccccgcaacatcttgcattactaccgtcacct |
| | | ccacagcccctactacttccaagaagaggcagcagcagcagaaaaagaccagcagaaaaccagcag |
| | | ctagaaaatccacagcggcggcagcaggtggactgaggatcgcggcgaacgagccggcgcaaacc |
| | | cgggagctgaggaaccggatctttcccaccctctatgccatcttccagcagagtcgggggcaggagca |
| | | ggaactgaaagtcaagaaccgttctctgcgctcgctcacccgcagttgtctgtatcacaagagcgaagac |
| | | caacttcagcgcactctcgaggacgccgaggctctcttcaacaagtactgcgcgctcactcttaaagagt |
| | | agcccgcgcccgcccagtcgcagaaaaaggcgggaattacgtcacctgtgcccttcgccctagccgcc |
| | | tccacccatcatcatgagcaaagagattcccacgccttacatgtggagctaccagccccagatgggcct |
| | | ggccgccggtgccgcccaggactactccacccgcatgaattggctcagcgccgggcccgcgatgatc |
| | | tcacgggtgaatgacatccgcgcccaccgaaaccagatactcctagaacagtcagcgctcaccgccac |
| | | gccccgcaatcacctcaatccgcgtaattggcccgccgccctggtgtaccaggaaattccccagcccac |
| | | gaccgtactacttccgcgagacgcccaggccgaagtccagctgactaactcaggtgtccagctggcgg |
| | | gcggcgccaccctgtgtcgtcaccgccccgctcagggtataaagcggctggtgatccggggcagagg |
| | | cacacagctcaacgacgaggtggtgagctcttcgctgggtctgcgacctgacggagtcttccaactcgc |
| | | cggatcggggagatcttccttcacgcctcgtcaggccgtcctgactttggagagttcgtcctcgcagccc |
| | | cgctcgggtggcatcggcactctccagttcgtggaggagttcactccctcggtctacttcaaccccttctc |
| | | cggctcccccggccactacccggacgagttcatcccgaacttcgacgccatcagcgagtcggtggacg |
| | | gctacgattgaaactaatcacccccttatccagtgaaataaagatcatattgatgatgattttacagaaataa |
| | | aaaataatcatttgatttgaaataaagatacaatcatattgatgatttgagtttaacaaaaaaaaaagaatca |
| | | cttacttgaaatctgataccaggtctctgtccatgttttctgccaacaccacttcactcccctcttcccagctct |
| | | ggtactgcaggccccggcgggctgcaaacttcctccacacgctgaaggggatgtcaaattcctcctgtc |
| | | cctcaatcttcattttatcttctatcagatgtccaaaaagcgcgtccgggtggatgatgacttcgaccccgtc |
| | | tacccctacgatgcagacaacgcaccgaccgtgcccttcatcaacccccccttcgtctcttcagatggatt |
| | | ccaagagaagcccctgggggtgttgtccctgcgactggccgaccccgtcaccaccaagaacggggaa |
| | | atcaccctcaagctgggagagggggtggacctcgattcctcgggaaaactcatctccaacacggccac |
| | | caaggccgccgcccctctcagtttttccaacaacaccatttcccttaacatggatcaccccttttacactaaa |
| | | gatggaaaattatccttacaagtttctccaccattaaatatactgagaacaagcattctaaacacactagcttt |
| | | aggttttggatcaggtttaggactccgtggctctgccttggcagtacagttagtctctccacttacatttgata |
| | | ctgatggaaacataaagcttaccttagacagaggtttgcatgttacaacaggagatgcaattgaaagcaa |
| | | cataagctgggctaaaggtttaaaatttgaagatggagccatagcaaccaacattggaaatgggttagag |
| | | tttggaagcagtagtacagaaacaggtgttgatgatgcttacccaatccaagttaaacttggatctggcctt |
| | | agctttgacagtacaggagccataatggctggtaacaaagaagacgataaactcactttgtggacaacac |
| | | ctgatccatcaccaaactgtcaaatactcgcagaaaatgatgcaaaactaacactttgcttgactaaatgtg |
| | | gtagtcaaatactggccactgtgtcagtcttagttgtaggaagtggaaacctaaaccccattactggcacc |
| | | gtaagcagtgctcaggtgtttctacgttttgatgcaaacggtgttcttttaacagaacattctacactaaaaaa |
| | | atactgggggtataggcagggagatagcatagatggcactccatataccaatgctgtaggattcatgccc |
| | | aatttaaaagcttatccaaagtcacaaagttctactactaaaaataatatagtagggcaagtatacatgaatg |
| | | gagatgtttcaaaacctatgcttctcactataaccctcaatggtactgatgacagcaacagtacatattcaat |
| | | gtcattttcatacacctggactaatggaagctatgttggagcaacatttggggctaactcttataccttctcat |
| | | acatcgcccaagaatgaacactgtatcccaccctgcatgccaacccttcccaccccactctgtggaacaa |
| | | actctgaaacacaaaataaaataaagttcaagtgttttattgattcaacagttttacaggattcgagcagttat |
| | | ttttcctccaccctcccaggacatggaatacaccaccctctccccccgcacagccttgaacatctgaatgc |
| | | cattggtgatggacatgcttttggtctccacgttccacacagtttcagagcgagccagtctcgggtcggtc |
| | | agggagatgaaaccctccgggcactcccgcatctgcacctcacagctcaacagctgaggattgtcctcg |
| | | gtggtcgggatcacggttatctggaagaagcagaagagcggcggtgggaatcatagtccgcgaacgg |
| | | gatcggccggtggtgtcgcatcaggccccgcagcagtcgctgccgccgccgctccgtcaagctgctgc |
| | | tcagggggtccgggtccagggactccctcagcatgatgcccacggccctcagcatcagtcgtctggtg |
| | | cggcgggcgcagcagcgcatgcggatctcgctcaggtcgctgcagtacgtgcaacacagaaccacca |
| | | ggttgttcaacagtccatagttcaacacgctccagccgaaactcatcgcgggaaggatgctacccacgtg |
| | | gccgtcgtaccagatcctcaggtaaatcaagtggtgccccctccagaacacgctgcccacgtacatgat |
| | | ctccttgggcatgtggcggttcaccacctcccggtaccacatcaccctctggttgaacatgcagccccgg |
| | | atgatcctgcggaaccacagggccagcaccgccccgcccgccatgcagcgaagagaccccgggtcc |
| | | cggcaatggcaatggaggacccaccgctcgtacccgtggatcatctgggagctgaacaagtctatgttg |
| | | gcacagcacaggcatatgctcatgcatctcttcagcactctcaactcctcgggggtcaaaaccatatccca |
| | | gggcacggggaactcttgcaggacagcgaaccccgcagaacagggcaatcctcgcacagaacttaca |
| | | ttgtgcatggacagggtatcgcaatcaggcagcaccgggtgatcctccaccagagaagcgcgggtctc |
| | | ggtctcctcacagcgtggtaagggggccggccgatacgggtgatggcgggacgcggctgatcgtgttc |
| | | gcgaccgtgtcatgatgcagttgctttcggacattttcgtacttgctgtagcagaacctggtccgggcgct |
| | | gcacaccgatcgccggcggcggtctcggcgcttggaacgctcggtgttgaaattgtaaaacagccactc |
| | | tctcagaccgtgcagcagatctagggcctcaggagtgatgaagatcccatcatgcctgatggctctgatc |
| | | acatcgaccaccgtggaatgggccagacccagccagatgatgcaattttgttgggtttcggtgacggcg |
| | | agcctcgggaacaacgatgaagtaaatgcaagcggtgcgttccagcatggttagttagctgatctgtaga |
| | | aaaaacaaaaatgaacattaaaccatgctagcctggcgaacaggtgggtaaatcgttctctccagcacca |
| | | ggcaggccacggggtctccggcgcgaccctcgtaaaaattgtcgctatgattgaaaaccatcacagag |
| | | agacgttcccggtggccggcgtgaatgattcgacaagatgaatacacccccggaacattggcgtccgc |
| | | gagtgaaaaaaagcgcccgaggaagcaataaggcactacaatgctcagtctcaagtccagcaaagcg |
| | | atgccatgoggatgaagcacaaaattctcaggtgcgtacaaaatgtaattactcccctcctgcacaggca |
| | | gcaaagcccccgatccctccaggtacacatacaaagcctcagcgtccatagcttaccgagcagcagca |
| | | cacaacaggcgcaagagtcagagaaaggctgagctctaacctgtccacccgctctctgctcaatatata |
| | | gcccagatctacactgacgtaaaggccaaagtctaaaaatacccgccaaataatcacacacgcccagca |
| | | cacgcccagaaaccggtgacacactcaaaaaaatacgcgcacttcctcaaacgcccaaaactgccgtc |
| | | atttccgggttcccacgctacgtcatgaaaacacgactttcaaattccgtcgaccgttaaaaacgtcaccc |
| | | gccccgcccctaacggtcgcccgtctctcagccaatcagegccccgcatccccaaattcaaacacctca |
| | | tttgcatattaacgcgcacaaaaagtttgaggtatattattgatgatg |

TABLE 12
SAM Vector Sequences

| SEQ ID | | |
|---|---|---|
| NO: | Description | Sequence |
| 522 | SAM-01 | atgggcggcgcatgagagaagcccagaccaattacctacccaaaatggagaaagttcacgttgacatcg |
| | (underline, | aggaagacagcccattcctcagagctttgcagcggagcttcccgcagtttgaggtagaagccaagcagg |
| | bold, | tcactgataatgaccatgctaatgccagagcgttttcgcatctggcttcaaaactgatcgaaacggaggtg |
| | uppercase = | gacccatccgacacgatccttgacattggaagtgcgcccgcccgcagaatgtattctaagcacaagtatc |
| | Immunogen | attgtatctgtccgatgagatgtgcggaagatccggacagattgtataagtatgcaactaagctgaagaaa |
| | (nt 7571 to | aactgtaaggaaataactgataaggaattggacaagaaaatgaaggagctcgccgccgtcatgagcga |
| | 10666); | ccctgacctggaaactgagactatgtgcctccacgacgacgagtcgtgtcgctacgaagggcaagtcgc |
| | Italic, bold, | tgtttaccaggatgtatacgcggttgacggaccgacaagtctctatcaccaagccaataagggagttaga |
| | uppercase = | gtcgcctactggataggctttgacaccaccccttttatgtttaagaacttggctggagcatatccatcatactc |
| | 26S sub- | taccaactgggccgacgaaaccgtgttaacggctcgtaacataggcctatgcagctctgacgttatggag |
| | genomic | cggtcacgtagagggatgtccattcttagaaagaagtatttgaaaccatccaacaatgttctattctctgttg |
| | promoter (nt | gctcgaccatctaccacgagaagagggacttactgaggagctggcacctgccgtctgtatttcacttacgt |
| | 7513 to 7536); | ggcaagcaaaattacacatgtcggtgtgagactatagttagttgcgacgggtacgtcgttaaaagaatagc |
| | dashed | tatcagtccaggcctgtatgggaagccttcaggctatgctgctacgatgcaccgcgagggattcttgtgct |
| | underline, | gcaaagtgacagacacattgaacggggagagggtctcttttcccgtgtgcacgtatgtgccagctacatt |
| | bold, | gtgtgaccaaatgactggcatactggcaacagatgtcagtgcggacgacgcgcaaaaactgctggttgg |
| | lowercase = | gctcaaccagcgtatagtcgtcaacggtcgcacccagagaaacaccaataccatgaaaaattaccttttg |
| | polyA region | cccgtagtggcccaggcatttgctaggtgggcaaaggaatataaggaagatcaagaagatgaaaggcc |
| | (nt 10953 to | actaggactacgagatagacagttagtcatggggtgttgttgggcttttagaaggcacaagataacatctat |
| | 11072)) | ttataagcgcccggatacccaaaccatcatcaaagtgaacagcgatttccactcattcgtgctgcccagga |
| | | taggcagtaacacattggagatcgggctgagaacaagaatcaggaaaatgttagaggagcacaaggag |
| | | ccgtcacctctcattaccgccgaggacgtacaagaagctaagtgcgcagccgatgaggctaaggaggt |
| | | gcgtgaagccgaggagttgcgcgcagctctaccacctttggcagctgatgttgaggagcccactctgga |
| | | agccgatgtcgacttgatgttacaagaggctggggccggctcagtggagacacctcgtggcttgataaa |
| | | ggttaccagctacgctggcgaggacaagatcggctcttacgctgtgctttctccgcaggctgtactcaaga |
| | | gtgaaaaattatcttgcatccaccctctcgctgaacaagtcatagtgataacacactctggccgaaaaggg |
| | | cgttatgccgtggaaccataccatggtaaagtagtggtgccagagggacatgcaatacccgtccaggac |
| | | tttcaagctctgagtgaaagtgccaccattgtgtacaacgaacgtgagttcgtaaacaggtacctgcacca |
| | | tattgccacacatggaggagcgctgaacactgatgaagaatattacaaaactgtcaagcccagcgagca |
| | | cgacggcgaatacctgtacgacatcgacaggaaacagtgcgtcaagaaagaactagtcactgggctag |
| | | ggctcacaggcgagctggtggatcctcccttccatgaattcgcctacgagagtctgagaacacgaccag |
| | | ccgctccttaccaagtaccaaccataggggtgtatggcgtgccaggatcaggcaagtctggcatcattaa |
| | | aagcgcagtcaccaaaaaagatctagtggtgagcgccaagaaagaaaactgtgcagaaattataaggg |
| | | acgtcaagaaaatgaaagggctggacgtcaatgccagaactgtggactcagtgctcttgaatggatgca |
| | | aacaccccgtagagaccctgtatattgacgaagcttttgcttgtcatgcaggtactctcagagcgctcatag |
| | | ccattataagacctaaaaaggcagtgctctgcggggatcccaaacagtgcggtttttttaacatgatgtgcc |
| | | tgaaagtgcattttaaccacgagatttgcacacaagtcttccacaaaagcatctctcgccgttgcactaaat |
| | | ctgtgacttcggtcgtctcaaccttgttttacgacaaaaaaatgagaacgacgaatccgaaagagactaag |
| | | attgtgattgacactaccggcagtaccaaacctaagcaggacgatctcattctcacttgtttcagagggtgg |
| | | gtgaagcagttgcaaatagattacaaaggcaacgaaataatgacggcagctgcctctcaagggctgacc |
| | | cgtaaaggtgtgtatgccgttcggtacaaggtgaatgaaaatcctctgtacgcacccacctcagaacatgt |
| | | gaacgtcctactgacccgcacggaggaccgcatcgtgtggaaaacactagccggcgacccatggataa |
| | | aaacactgactgccaagtaccctgggaatttcactgccacgatagaggagtggcaagcagagcatgatg |
| | | ccatcatgaggcacatcttggagagaccggaccctaccgacgtcttccagaataaggcaaacgtgtgttg |
| | | ggccaaggctttagtgccggtgctgaagaccgctggcatagacatgaccactgaacaatggaacactgt |
| | | ggattattttgaaacggacaaagctcactcagcagagatagtattgaaccaactatgcgtgaggttctttgg |
| | | actcgatctggactccggtctattttctgcacccactgttccgttatccattaggaataatcactgggataact |
| | | ccccgtcgcctaacatgtacgggctgaataaagaagtggtccgtcagctctctcgcaggtacccacaact |
| | | gcctcgggcagttgccactggaagagtctatgacatgaacactggtacactgcgcaattatgatccgcgc |
| | | ataaacctagtacctgtaaacagaagactgcctcatgctttagtcctccaccataatgaacacccacagag |
| | | tgacttttcttcattcgtcagcaaattgaagggcagaactgtcctggtggtcggggaaaagttgtccgtccc |
| | | aggcaaaatggttgactggttgtcagaccggcctgaggctaccttcagagctcggctggatttaggcatc |
| | | ccaggtgatgtgcccaaatatgacataatatttgttaatgtgaggaccccatataaataccatcactatcagc |
| | | agtgtgaagaccatgccattaagcttagcatgttgaccaagaaagcttgtctgcatctgaatcccggcgga |
| | | acctgtgtcagcataggttatggttacgctgacagggccagcgaaagcatcattggtgctatagcgcggc |
| | | agttcaagttttcccgggtatgcaaaccgaaatcctcacttgaagagacggaagttctgtttgtattcattgg |
| | | gtacgatcgcaaggcccgtacgcacaatccttacaagctttcatcaaccttgaccaacatttatacaggttc |
| | | cagactccacgaagccggatgtgcaccctcatatcatgtggtgcgaggggatattgccacggccaccga |
| | | aggagtgattataaatgctgctaacagcaaaggacaacctggcggaggggtgtgcggagcgctgtataa |
| | | gaaattcccggaaagcttcgatttacagccgatcgaagtaggaaaagcgcgactggtcaaaggtgcagc |
| | | taaacatatcattcatgccgtaggaccaaacttcaacaaagtttcggaggttgaaggtgacaaacagttgg |
| | | cagaggcttatgagtccatcgctaagattgtcaacgataacaattacaagtcagtagcgattccactgttgt |
| | | ccaccggcatcttttccgggaacaaagatcgactaacccaatcattgaaccatttgctgacagctttagaca |
| | | ccactgatgcagatgtagccatatactgcagggacaagaaatgggaaatgactctcaaggaagcagtgg |
| | | ctaggagagaagcagtggaggagatatgcatatccgacgactcttcagtgacagaacctgatgcagagc |
| | | tggtgagggtgcatccgaagagttctttggctggaaggaagggctacagcacaagcgatggcaaaactt |
| | | tctcatatttggaagggaccaagtttcaccaggcggccaaggatatagcagaaattaatgccatgtggcc |
| | | cgttgcaacggaggccaatgagcaggtatgcatgtatatcctcggagaaagcatgagcagtattaggtcg |
| | | aaatgccccgtcgaagagtcggaagcctccacaccacctagcacgctgccttgcttgtgcatccatgcca |
| | | tgactccagaaagagtacagcgcctaaaagcctcacgtccagaacaaattactgtgtgctcatcctttccat |
| | | tgccgaagtatagaatcactggtgtgcagaagatccaatgctcccagcctatattgttctcaccgaaagtg |
| | | cctgcgtatattcatccaaggaagtatctcgtggaaacaccaccggtagacgagactccggagccatcg |
| | | gcagagaaccaatccacagaggggacacctgaacaaccaccacttataaccgaggatgagaccagga |
| | | ctagaacgcctgagccgatcatcatcgaagaggaagaagaggatagcataagtttgctgtcagatggcc |
| | | cgacccaccaggtgctgcaagtcgaggcagacattcacgggccgccctctgtatctagctcatcctggtc |
| | | cattcctcatgcatccgactttgatgtggacagtttatccatacttgacaccctggagggagctagcgtgac |
| | | cagcggggcaacgtcagccgagactaactcttacttcgcaaagagtatggagtttctggcgcgaccggt |
| | | gcctgcgcctcgaacagtattcaggaaccctccacatcccgctccgcgcacaagaacaccgtcacttgc |
| | | acccagcagggcctgctcgagaaccagcctagtttccaccccgccaggcgtgaatagggtgatcacta |
| | | gagaggagctcgaggcgcttaccccgtcacgcactcctagcaggtcggtctcgagaaccagcctggtct |
| | | ccaacccgccaggcgtaaatagggtgattacaagagaggagtttgaggcgttcgtagcacaacaacaat |
| | | gacggtttgatgcgggtgcatacatcttttcctccgacaccggtcaagggcatttacaacaaaaatcagta |
| | | aggcaaacggtgctatccgaagtggtgttggagaggaccgaattggagatttcgtatgccccgcgcctc |
| | | gaccaagaaaaagaagaattactacgcaagaaattacagttaaatcccacacctgctaacagaagcaga |
| | | taccagtccaggaaggtggagaacatgaaagccataacagctagacgtattctgcaaggcctagggcat |
| | | tatttgaaggcagaaggaaaagtggagtgctaccgaaccctgcatcctgttcctttgtattcatctagtgtga |
| | | accgtgccttttcaagccccaaggtcgcagtggaagcctgtaacgccatgttgaaagagaactttccgact |
| | | gtggcttcttactgtattattccagagtacgatgcctatttggacatggttgacggagcttcatgctgcttaga |
| | | cactgccagtttttgccctgcaaagctgcgcagctttccaaagaaacactcctatttggaacccacaatacg |
| | | atcggcagtgccttcagcgatccagaacacgctccagaacgtcctggcagctgccacaaaaagaaattg |
| | | caatgtcacgcaaatgagagaattgcccgtattggattcggcggcctttaatgtggaatgcttcaagaaata |
| | | tgcgtgtaataatgaatattgggaaacgtttaaagaaaaccccatcaggcttactgaagaaaacgtggtaa |
| | | attacattaccaaattaaaaggaccaaaagctgctgctctttttgcgaagacacataatttgaatatgttgca |
| | | ggacataccaatggacaggtttgtaatggacttaaagagagacgtgaaagtgactccaggaacaaaaca |
| | | tactgaagaacggcccaaggtacaggtgatccaggctgccgatccgctagcaacagcgtatctgtgcgg |
| | | aatccaccgagagctggttaggagattaaatgcggtcctgcttccgaacattcatacactgtttgatatgtc |
| | | ggctgaagactttgacgctattatagccgagcacttccagcctggggattgtgttctggaaactgacatcg |
| | | cgtcgtttgataaaagtgaggacgacgccatggctctgaccgcgttaatgattctggaagacttaggtgtg |
| | | gacgcagagctgttgacgctgattgaggcggctttcggcgaaatttcatcaatacatttgcccactaaaact |
| | | aaatttaaattcggagccatgatgaaatctggaatgttcctcacactgtttgtgaacacagtcattaacattgt |
| | | aatcgcaagcagagtgttgagagaacggctaaccggatcaccatgtgcagcattcattggagatgacaat |
| | | atcgtgaaaggagtcaaatcggacaaattaatggcagacaggtgcgccacctggttgaatatggaagtc |
| | | aagattatagatgctgtggtgggcgagaaagcgccttatttctgtggagggtttattttgtgtgactccgtga |
| | | ccggcacagcgtgccgtgtggcagaccccctaaaaaggctgtttaagcttggcaaacctctggcagcag |
| | | acgatgaacatgatgatgacaggagaagggcattgcatgaagagtcaacacgctggaaccgagtgggt |
| | | attctttcagagctgtgcaaggcagtagaatcaaggtatgaaaccgtaggaacttccatcatagttatggcc |
| | | atgactactctagctagcagtgttaaatcattcagctacctgagaggggcccctataaCTCTCTACG |
| | | GCTAACCTGAATGGActacgactctagaatagtctttaatTAAGCCACCATGAT |
| | | TTCTCCTCGGACGCTGAATGCATGGGTGAAGGTAGTGGAG |
| | | GAAAAGGCATTTTCACCAGAAGTCATTCCGATGTTCTCCGC |
| | | GCTCTCCGAGGGTGCTACGCCACAGGACTTGAATACGATGC |
| | | TGAATACCGTTGGTGGCCATCAGGCGGCGATGCAGATGTT |
| | | GAAGGAGACAATTAACGAAGAAGCCGCCGAATGGGACAGA |
| | | TTGCACCCGGTGCATGCGGGGCCAATAGCTCCTGGCCAGA |
| | | TGCGCGAGCCTAGGGGTTCTGACATTGCTGGTACAACAAGT |
| | | ACCCTTCAGGAGCAGATTGGTTGGATGACGAATAACCCTCC |
| | | CATACCTGTTGGCGAGATCTATAAGCGCTGGATTATACTTG |
| | | GGCTGAATAAGATAGTCCGAATGTATTCTCCCACCTCTATT |
| | | CTGGATATTCGGCAAGGACCTAAGGAGCCGTTTAGAGACTA |
| | | CGTAGACCGGTTTTACAAAACCCTGCGGGCGGAACAAGCTT |
| | | CTCAGGAAGTCAAAAATTGGATGACTGAGACCTTGCTCGTC |
| | | CAGAATGCGAACCCGGACTGTAAAACAATACTCAAAGCGCT |
| | | GGGCCCCGCTGCAACCCTGGAAGAAATGATGACGGCTTGT |
| | | CAGGGAGTAGGAGGCCCCGGACATAAGGCACGAGTGTTGG |
| | | CAGAAGCCATGAGCCAGCCGCCTGTCGTCGCGAAAGAAAT |
| | | CGTCGCTTCTTGCGACAAATGTCAGCTGAAGGGGGAGGCG |
| | | ATGCACGGTCAAGTTGATTGCTCTCCCGGTATTTGGCAATT |
| | | GGACTGTACCCACCTTGAAGGCAAAATTATTCTGGTTGCAG |
| | | TGCACGTAGCATCCGGTTACATCGAAGCTGAAGTGATACCC |
| | | GCAGAGACAGGCCAGGAGACGGCTTATTTCCTCCTTAAGCT |
| | | TGCGGGTCGGTGGCCCGTAAAGACCATGGCTGCTCGGGCA |
| | | TCTGTCCTCTCCGGTGGTGAACTCGACCGATGGGAAAAGAT |
| | | TCGATTGCGCCCCGGAGGAAAGAAGAAATATAGGCTGAAA |
| | | CATATTGTGTGGGCATCACGGGAACTTGAGCGATTTGCGGT |
| | | AAACCCAGGCCTTTTGGAAACAGCTGCAACTGTGAAAGCGG |
| | | CTTGCTGGTGGGCGGGGATAAAACAGGAGTTTGGTATCCC |
| | | GTACAATCCCCAATCTCAGGGGGTAGTAGAAAGCATGAACA |
| | | AAGAATTGAAAAAAATAATTGGCCAGGTTCGCGACCAAGCC |
| | | GAGCACCTCAAGACCGCTGTACAGATGGCTGTATTTATTCA |
| | | CAACTTCAAGCGGAAGGGCGGAATAGGAGGATATAGCGCA |
| | | GGGGAAAGGATTGTTGATATTATTGCAACACAAGGTTACTT |
| | | TCCTGACTGGCAAAACTACACACCGGGCCCTGGCACGCGCT |
| | | ATCCCCTTACGTTCGGTTGGTGCTTCAAGCTGGTGCCGGTA |
| | | AAAGAAAAAGTTTATTTGGCATGGGTTCCTGCACATAAAGG |
| | | AATAGGGGGTAACGAACAAGTTGACAAACTCGTCAGCCGC |
| | | GCTAAAAGAGCCCCAGTCAAGCAGACCCTGAATTTTGACCT |
| | | GCTTAAATTGGCTGGGGACGTCGAGAGTAACCCGGGACCC |
| | | TTCCCACAAATTACACTCTGGCAGCGACCACTGGTAACAAT |
| | | CAAAATAGGGGGACAATTGAAAGAAGCACTCCTGGATACG |
| | | GGCGCGGACGATACAGTCCTGGAGGAAATGAATCTCCCCG |
| | | GCCGCTGGAAACCTAAGATGATAGGGGGGATCGGTGGATT |
| | | TATTAAAGTGCGGCAGTACGATCAATGGGGTTTTACGACAC |
| | | CTGACAAGAAACATCAAAAGGAGCCGCCATTTCTTTGGATG |
| | | GGTTATGAGCTTCATCCGGATAAATGGACTGTTCAGCCGAT |
| | | TGTCCTCCCCGAGAAGGATAGTTGGACTGTGAACGACATCC |
| | | AGAAGCTGGTCGGGAAACTTAATTGGGCCAGTCAAATATAT |
| | | CCAGGTATTAAAGTTGCCGCACAAGAAGAGGAGGAAGTAG |
| | | GGTTCCCCGTGAAACCGCAAGTCCCTCTCCGGCCCATGACC |
| | | TTTAAGGGCGCTCTCGACCTGTCCCATTTCCTCCGCGAAAA |
| | | GGGGGGTTTGGAAGGCTTGATCATTACCAAGATTCAAAACT |
| | | TCAGGGTCTATTATCGAGACAGTCGCGATCCCCTTTGGAAA |
| | | GGACCTGCGAAACTTCTTTGGAAAGGAGAAGGAGCCGTGG |
| | | TAATTCAGGATAATTCTGACATAAAGGTCGTCCCACGCCGA |
| | | AAAGCGAAGATTATAAGAGATTATGGCAAGCAGATGGCCG |
| | | GGGATGATTGTGTCGCAAGTAGACAGGATGAAGACCCTAA |
| | | ATTCAAGCTTCCAATCCAGAAGGAAACGTGGGAGACATGGT |
| | | GGACCGAGTATTGGCAAGCTACTTGGATCCCAGAATGGGA |
| | | ATTTGTGAACACTCCCCCACTCGTAAAGCTGTGGTACCAGC |
| | | TTGAAAAAGAACCTATAGTCGGGGCGGAGACGTTCTATGTA |
| | | GACGGCGCCGCTAATCGAGAGACAAAAGCAGCAGGCACGG |
| | | TACTGGTAGGCCCGACCCCTGTCAACATCATTGGACGAAAT |
| | | CTGTTGACCCAGATTGGGTGTACCCTTAACTTTCCCATTTC |
| | | ACCAATAGAGACCGTCCCGGTTAAACTGAAACCGGGTATGG |
| | | ATGGTCCCAAAGTAAAACAGTGGCCACTTACCGAGGAGAA |
| | | GATTAAGGCACTCGTTGAAATATGTACAGAAATGGAAAAAG |
| | | AGGGGAAAATCTCTAAAATTGGCCCTGAAAATCCGTACAAC |
| | | ACTCCGGTATTCGCCATAAAAAAGAAGGACTCTACCAAGTG |
| | | GCGCAAACTCGTGGACTTTAGAGAACTGAATAAAAGGACCC |
| | | AGGACTTTTGGGAAGTCCAGCTGGGTATTCCTCACCCCGCT |
| | | GGTCTCAAGAAAAAGAAAAGTGTCACTGTCTTGGATGTTGG |
| | | AGATGCGTACTTTTCAGTACCTCTTGATAAAGATTTTCGAA |
| | | AGTATACCGCGTTTACCATTCCCTCCATAAATAACGAAACA |
| | | CCGGGGATCAGGTATCAATATAACGTGCTTCCACAAGGCTG |
| | | GAAGGGTTCACCGGCTATTTTCCAATCTTCTATGACGTGAta |
| | | gttcgaacggccgtatcacgcccaaacatttacagccgcggtgtcaaaaaccgcgtggacgtggttaac |
| | | atccctgctgggaggatcagccgtaattattataattggcttggtgctggctactattgtggccatgtacgtg |
| | | ctgaccaaccagaaacataattgaatacagcagcaattggcaagctgcttacatagaactcgcggcgatt |
| | | 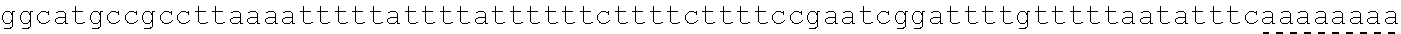 |
| | | 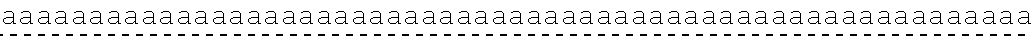 |
| | | 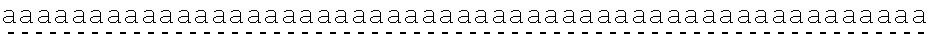 |
| 523 | SAM-02 | atgggcggcgcatgagagaagcccagaccaattacctacccaaaatggagaaagttcacgttgacatcg |
| | (underline, | aggaagacagcccattcctcagagctttgcagcggagcttcccgcagtttgaggtagaagccaagcagg |
| | bold, | tcactgataatgaccatgctaatgccagagcgttttcgcatctggcttcaaaactgatcgaaacggaggtg |
| | uppercase = | gacccatccgacacgatccttgacattggaagtgcgcccgcccgcagaatgtattctaagcacaagtatc |
| | Immunogen | attgtatctgtccgatgagatgtgcggaagatccggacagattgtataagtatgcaactaagctgaagaaa |
| | (nt 7571 to | aactgtaaggaaataactgataaggaattggacaagaaaatgaaggagctcgccgccgtcatgagcga |
| | 10663); | ccctgacctggaaactgagactatgtgcctccacgacgacgagtcgtgtcgctacgaagggcaagtcgc |
| | Italic, bold, | tgtttaccaggatgtatacgcggttgacggaccgacaagtctctatcaccaagccaataagggagttaga |
| | uppercase = | gtcgcctactggataggctttgacaccaccccttttatgtttaagaacttggctggagcatatccatcatactc |
| | 26S sub- | taccaactgggccgacgaaaccgtgttaacggctcgtaacataggcctatgcagctctgacgttatggag |
| | genomic | cggtcacgtagagggatgtccattcttagaaagaagtatttgaaaccatccaacaatgttctattctctgttg |
| | promoter (nt | gctcgaccatctaccacgagaagagggacttactgaggagctggcacctgccgtctgtatttcacttacgt |
| | 7513 to 7536; | ggcaagcaaaattacacatgtcggtgtgagactatagttagttgcgacgggtacgtcgttaaaagaatagc |
| | dashed | tatcagtccaggcctgtatgggaagccttcaggctatgctgctacgatgcaccgcgagggattcttgtgct |
| | underline, | gcaaagtgacagacacattgaacggggagagggtctcttttcccgtgtgcacgtatgtgccagctacatt |
| | bold, | gtgtgaccaaatgactggcatactggcaacagatgtcagtgcggacgacgcgcaaaaactgctggttgg |
| | lowercase = | gctcaaccagcgtatagtcgtcaacggtcgcacccagagaaacaccaataccatgaaaaattaccttttg |
| | polyA region | cccgtagtggcccaggcatttgctaggtgggcaaaggaatataaggaagatcaagaagatgaaaggcc |
| | (nt 10950 to | actaggactacgagatagacagttagtcatggggtgttgttgggcttttagaaggcacaagataacatctat |
| | 11069)) | ttataagcgcccggatacccaaaccatcatcaaagtgaacagcgatttccactcattcgtgctgcccagga |
| | | taggcagtaacacattggagatcgggctgagaacaagaatcaggaaaatgttagaggagcacaaggag |
| | | ccgtcacctctcattaccgccgaggacgtacaagaagctaagtgcgcagccgatgaggctaaggaggt |
| | | gcgtgaagccgaggagttgcgcgcagctctaccacctttggcagctgatgttgaggagcccactctgga |
| | | agccgatgtcgacttgatgttacaagaggctggggccggctcagtggagacacctcgtggcttgataaa |
| | | ggttaccagctacgctggcgaggacaagatcggctcttacgctgtgctttctccgcaggctgtactcaaga |
| | | gtgaaaaattatcttgcatccaccctctcgctgaacaagtcatagtgataacacactctggccgaaaaggg |
| | | cgttatgccgtggaaccataccatggtaaagtagtggtgccagagggacatgcaatacccgtccaggac |
| | | tttcaagctctgagtgaaagtgccaccattgtgtacaacgaacgtgagttcgtaaacaggtacctgcacca |
| | | tattgccacacatggaggagcgctgaacactgatgaagaatattacaaaactgtcaagcccagcgagca |
| | | cgacggcgaatacctgtacgacatcgacaggaaacagtgcgtcaagaaagaactagtcactgggctag |
| | | ggctcacaggcgagctggtggatcctcccttccatgaattcgcctacgagagtctgagaacacgaccag |
| | | ccgctccttaccaagtaccaaccataggggtgtatggcgtgccaggatcaggcaagtctggcatcattaa |
| | | aagcgcagtcaccaaaaaagatctagtggtgagcgccaagaaagaaaactgtgcagaaattataaggg |
| | | acgtcaagaaaatgaaagggctggacgtcaatgccagaactgtggactcagtgctcttgaatggatgca |
| | | aacaccccgtagagaccctgtatattgacgaagcttttgcttgtcatgcaggtactctcagagcgctcatag |
| | | ccattataagacctaaaaaggcagtgctctgcggggatcccaaacagtgcggtttttttaacatgatgtgcc |
| | | tgaaagtgcattttaaccacgagatttgcacacaagtcttccacaaaagcatctctcgccgttgcactaaat |
| | | ctgtgacttcggtcgtctcaaccttgttttacgacaaaaaaatgagaacgacgaatccgaaagagactaag |
| | | attgtgattgacactaccggcagtaccaaacctaagcaggacgatctcattctcacttgtttcagaggggg |
| | | gtgaagcagttgcaaatagattacaaaggcaacgaaataatgacggcagctgcctctcaagggctgacc |
| | | cgtaaaggtgtgtatgccgttcggtacaaggtgaatgaaaatcctctgtacgcacccacctcagaacatgt |
| | | gaacgtcctactgacccgcacggaggaccgcatcgtgtggaaaacactagccggcgacccatggataa |
| | | aaacactgactgccaagtaccctgggaatttcactgccacgatagaggagtggcaagcagagcatgatg |
| | | ccatcatgaggcacatcttggagagaccggaccctaccgacgtcttccagaataaggcaaacgtgtgttg |
| | | ggccaaggctttagtgccggtgctgaagaccgctggcatagacatgaccactgaacaatggaacactgt |
| | | ggattattttgaaacggacaaagctcactcagcagagatagtattgaaccaactatgcgtgaggttctttgg |
| | | actcgatctggactccggtctattttctgcacccactgttccgttatccattaggaataatcactgggataact |
| | | ccccgtcgcctaacatgtacgggctgaataaagaagtggtccgtcagctctctcgcaggtacccacaact |
| | | gcctcgggcagttgccactggaagagtctatgacatgaacactggtacactgcgcaattatgatccgcgc |
| | | ataaacctagtacctgtaaacagaagactgcctcatgctttagtcctccaccataatgaacacccacagag |
| | | tgacttttcttcattcgtcagcaaattgaagggcagaactgtcctggtggtcggggaaaagttgtccgtccc |
| | | aggcaaaatggttgactggttgtcagaccggcctgaggctaccttcagagctcggctggatttaggcatc |
| | | ccaggtgatgtgcccaaatatgacataatatttgttaatgtgaggaccccatataaataccatcactatcagc |
| | | agtgtgaagaccatgccattaagcttagcatgttgaccaagaaagcttgtctgcatctgaatcccggcgga |
| | | acctgtgtcagcataggttatggttacgctgacagggccagcgaaagcatcattggtgctatagcgcggc |
| | | agttcaagttttcccgggtatgcaaaccgaaatcctcacttgaagagacggaagttctgtttgtattcattgg |
| | | gtacgatcgcaaggcccgtacgcacaatccttacaagctttcatcaaccttgaccaacatttatacaggttc |
| | | cagactccacgaagccggatgtgcaccctcatatcatgtggtgcgaggggatattgccacggccaccga |
| | | aggagtgattataaatgctgctaacagcaaaggacaacctggcggaggggtgtgcggagcgctgtataa |
| | | gaaattcccggaaagcttcgatttacagccgatcgaagtaggaaaagcgcgactggtcaaaggtgcagc |
| | | taaacatatcattcatgccgtaggaccaaacttcaacaaagtttcggaggttgaaggtgacaaacagttgg |
| | | cagaggcttatgagtccatcgctaagattgtcaacgataacaattacaagtcagtagcgattccactgttgt |
| | | ccaccggcatcttttccgggaacaaagatcgactaacccaatcattgaaccatttgctgacagctttagaca |
| | | ccactgatgcagatgtagccatatactgcagggacaagaaatgggaaatgactctcaaggaagcagtgg |
| | | ctaggagagaagcagtggaggagatatgcatatccgacgactcttcagtgacagaacctgatgcagagc |
| | | tggtgagggtgcatccgaagagttctttggctggaaggaagggctacagcacaagcgatggcaaaactt |
| | | tctcatatttggaagggaccaagtttcaccaggcggccaaggatatagcagaaattaatgccatgtggcc |
| | | cgttgcaacggaggccaatgagcaggtatgcatgtatatcctcggagaaagcatgagcagtattaggtcg |
| | | aaatgccccgtcgaagagtcggaagcctccacaccacctagcacgctgccttgcttgtgcatccatgcca |
| | | tgactccagaaagagtacagcgcctaaaagcctcacgtccagaacaaattactgtgtgctcatcctttccat |
| | | tgccgaagtatagaatcactggtgtgcagaagatccaatgctcccagcctatattgttctcaccgaaagtg |
| | | cctgcgtatattcatccaaggaagtatctcgtggaaacaccaccggtagacgagactccggagccatcg |
| | | gcagagaaccaatccacagaggggacacctgaacaaccaccacttataaccgaggatgagaccagga |
| | | ctagaacgcctgagccgatcatcatcgaagaggaagaagaggatagcataagtttgctgtcagatggcc |
| | | cgacccaccaggtgctgcaagtcgaggcagacattcacgggccgccctctgtatctagctcatcctggtc |
| | | cattcctcatgcatccgactttgatgtggacagtttatccatacttgacaccctggagggagctagcgtgac |
| | | cagcggggcaacgtcagccgagactaactcttacttcgcaaagagtatggagtttctggcgcgaccggt |
| | | gcctgcgcctcgaacagtattcaggaaccctccacatcccgctccgcgcacaagaacaccgtcacttgc |
| | | acccagcagggcctgctcgagaaccagcctagtttccaccccgccaggcgtgaatagggtgatcacta |
| | | gagaggagctcgaggcgcttaccccgtcacgcactcctagcaggtcggtctcgagaaccagcctggtct |
| | | ccaacccgccaggcgtaaatagggtgattacaagagaggagtttgaggcgttcgtagcacaacaacaat |
| | | gacggtttgatgcgggtgcatacatcttttcctccgacaccggtcaagggcatttacaacaaaaatcagta |
| | | aggcaaacggtgctatccgaagtggtgttggagaggaccgaattggagatttcgtatgccccgcgcctc |
| | | gaccaagaaaaagaagaattactacgcaagaaattacagttaaatcccacacctgctaacagaagcaga |
| | | taccagtccaggaaggtggagaacatgaaagccataacagctagacgtattctgcaaggcctagggcat |
| | | tatttgaaggcagaaggaaaagtggagtgctaccgaaccctgcatcctgttcctttgtattcatctagtgtga |
| | | accgtgccttttcaagccccaaggtcgcagtggaagcctgtaacgccatgttgaaagagaactttccgact |
| | | gtggcttcttactgtattattccagagtacgatgcctatttggacatggttgacggagcttcatgctgcttaga |
| | | cactgccagtttttgccctgcaaagctgcgcagctttccaaagaaacactcctatttggaacccacaatacg |
| | | atcggcagtgccttcagcgatccagaacacgctccagaacgtcctggcagctgccacaaaaagaaattg |
| | | caatgtcacgcaaatgagagaattgcccgtattggattcggcggcctttaatgtggaatgcttcaagaaata |
| | | tgcgtgtaataatgaatattgggaaacgtttaaagaaaaccccatcaggcttactgaagaaaacgtggtaa |
| | | attacattaccaaattaaaaggaccaaaagctgctgctctttttgcgaagacacataatttgaatatgttgca |
| | | ggacataccaatggacaggtttgtaatggacttaaagagagacgtgaaagtgactccaggaacaaaaca |
| | | tactgaagaacggcccaaggtacaggtgatccaggctgccgatccgctagcaacagcgtatctgtgcgg |
| | | aatccaccgagagctggttaggagattaaatgcggtcctgcttccgaacattcatacactgtttgatatgtc |
| | | ggctgaagactttgacgctattatagccgagcacttccagcctggggattgtgttctggaaactgacatcg |
| | | cgtcgtttgataaaagtgaggacgacgccatggctctgaccgcgttaatgattctggaagacttaggtgtg |
| | | gacgcagagctgttgacgctgattgaggcggctttcggcgaaatttcatcaatacatttgcccactaaaact |
| | | aaatttaaattcggagccatgatgaaatctggaatgttcctcacactgtttgtgaacacagtcattaacattgt |
| | | aatcgcaagcagagtgttgagagaacggctaaccggatcaccatgtgcagcattcattggagatgacaat |
| | | atcgtgaaaggagtcaaatcggacaaattaatggcagacaggtgcgccacctggttgaatatggaagtc |
| | | aagattatagatgctgtggtgggcgagaaagcgccttatttctgtggagggtttattttgtgtgactccgtga |
| | | ccggcacagcgtgccgtgtggcagaccccctaaaaaggctgtttaagcttggcaaacctctggcagcag |
| | | acgatgaacatgatgatgacaggagaagggcattgcatgaagagtcaacacgctggaaccgagtgggt |
| | | attctttcagagctgtgcaaggcagtagaatcaaggtatgaaaccgtaggaacttccatcatagttatggcc |
| | | atgactactctagctagcagtgttaaatcattcagctacctgagaggggcccctataaCTCTCTACG |
| | | GCTAACCTGAATGGActacgactctagaatagtctttaattaagccaccATGTTGTC |
| | | TCCTAGAACTCTGAATGCTTGGGTAAAGGTGATCGAAGAAA |
| | | AGGCATTCTCACCCGAGGTTATCCCTATGTTTACTGCGTTG |
| | | AGCGAAGGCGCAACACCCCATGATCTGAACACAATGCTGAA |
| | | TACAATCGGCGGACATCAAGCTGCTATGCAAATGCTTAAGG |
| | | ACACCATCAATGAGGAGGCAGCCGAGTGGGATCGCGTTCA |
| | | TCCAGTCCACGCTGGGCCCGTTGCGCCTGGTCAGATGAGG |
| | | GACCCACGAGGATCCGACATCGCAGGGAGCACCAGTACAC |
| | | TCCAAGAGCAGATTGCATGGATGACGAACAATCCACCAATA |
| | | CCTGTCGGTGACATTTACAAAAGGTGGATTATTATGGGCTT |
| | | GAACAAAATTGTACGGATGTATAGCCCGGTGAGCATACTGG |
| | | ACATTAAACAGGGTCCAAAAGAACCCTTTCGAGATTACGTT |
| | | GACAGATTTTATAGGACACTGAGGGCGGAACAAGCGTCTCA |
| | | AGACGTTAAAAACTGGATGACAGAAACGCTGCTTGTACAGA |
| | | ATTCCAACCCAGACTGCAAAACTATACTGAAAGCGCTCGGT |
| | | CCCGGTGCGACACTTGAGGAAATGATGAGCGCATGCCAGG |
| | | GCGTCGGAGGGCCGTCTCACAAGGCCCGCGTGTTGGCGGA |
| | | AGCTATGTGCCAGGTGGCTAAGGAGATAGTAGCATGTTGTG |
| | | ATAAATGTCAGCTTAAAGGTGAAGCTATACATGGGCAGGTG |
| | | GATTGTAGCCCGGGTGTATGGCAGCTGGACTGTACTCATCT |
| | | GGAAGGAAAGGTAATACTTGTCGCAGTTCATGTCGCGAGC |
| | | GGATACATTGAAGCTGAAATCATTCCTACGGAGACGGGCCA |
| | | AGAGACAGCTTACTTCATACTTAAACTTGCTGGGCGATGGC |
| | | CGGTGACAACTATGGCGGCGCGCGCTTCAATTTTGAGTGGT |
| | | GGAAAGTTGGACAAGTGGGAGAAGATTAGACTCAGACCCG |
| | | GAGGGAGAAAGAAGTATAAACTGAAACATCTGGTTTGGGCT |
| | | TCACGCGAACTTGAACGGTTTGCTCTCAACCCCGGGCTGCT |
| | | CGAAACTGCTGCTGCTGTAAAGGCCGCTTGTTGGTGGGCG |
| | | GGGGTAAAGCAGGAATTCGGAATTCCATATAACACTCAAAG |
| | | CCAGGGAGTAGTGGAATCCATGAATAATGAACTTAAGAAGA |
| | | TAATTGGACAGATTCGCGATCAGGCTGAACATCTCAAGACG |
| | | GCCGTACAAATGGCAGTATTGATTCATAACTTTAAACGGAA |
| | | GGGCGGCATAGGAGAGTATTCTGCGGGAGAACGCATAATA |
| | | GATATAATTGCGACTCAGGGGTTCTTTCCGGATTGGCAGAA |
| | | CTATACGCCGGGGCCAGGCATTAGGTTCCCCCTCACGTTTG |
| | | GATGGTGTTTCAAGTTGGTACCGTTGCTCATTAAAAAAGAA |
| | | AAAATCTACCTGGCCTGGGTCCCGGCGCACAAGGGTATAG |
| | | GGGGGAACGAGCAAATTGATAAGCTCGTGTCAAGGGCGAA |
| | | GCGCGCGCCAGTCAAACAGACCTTGAATTTCGACCTCCTTA |
| | | AGCTCGCTGGAGACGTCGAATCCAACCCTGGCCCGATGCT |
| | | GCCACAAATCACATTGTGGCAACGACCCATTGTAACAATAA |
| | | AAATCGGGGGCCAGATCAAAGAAGCGCTGCTTGACACCGG |
| | | CGCCGACGATACAGTCCTCGAGGATATGAATTTGCCAGGCA |
| | | AATGGAAGCCGAAGATGATTGGCGGCATTGGCGGCTTTATT |
| | | AAGGTTAAACAGTATGATCAGTGGGGATTGACCACTCCCGA |
| | | TAAGAAGCATCAGAAAGATCCGCCTTTTCTGTGGATGGGGT |
| | | ACGAACTGCACCCTGATCGATGGACGGTCCAGCCGATAGA |
| | | GCTGCCGGAAAAAGAATCATGGACCGTGAATGATATTCAAA |
| | | AACTGATCGGAAAACTCAATTGGGCGTCCCAGATATATGCT |
| | | GGCATCAAAGTTGCAGCACAAGAAGAGGAAGAGGTAGGTT |
| | | TCCCGGTTCGGCCGCAAGTTCCCTTGCGACCGATGACATAC |
| | | AAGGGCGCATTGGACCTTTCTCACTTCCTCAAGGAAAAGGG |
| | | CGGTTTGGAGGGCATCACTAAACTTCAGAATTTCAGAGTCT |
| | | ACTATAGAGATAACAGGGACCCATTGTGGAAGGGCCCCGC |
| | | TCGACTTCTCTGGAAAGGGGAGGGAGCGGTTGTAATTCAA |
| | | GACAACAGTGAAATTAAGGTCGTCCCACGACGGAAGGTTAA |
| | | AATAATTCGCGACTATGGCAAGCGAATGGCGGGAGACGAC |
| | | TGTGTAGCAGGACGACAAGACGAGGACCCAAAGTTTAGATT |
| | | GCCGATCCAGAAAGAGACATGGGATACGTGGTGGACGGAC |
| | | TATTGGCAGGCCACCTGGATACCAGAATGGGAGTTTACAAA |
| | | CACTCCTCCACTCGTGAAATTGTGGTATCAACTTGAGACCG |
| | | AGCCCATAGCTGGTGTAGAGACGTTTTACGTTGACGGTGCT |
| | | AGCAACAGGGAAACAAAGGCCGCTGGAACCGTGCTCATCG |
| | | GTCCTACTCCTGTGAACATAATTGGACGAAATTTGTTGACC |
| | | CAGCTGGGATGCACCCTCAATTTCCCCATTAGCCCAATAGA |
| | | TACCGTACCAGTCAAGCTTAAGCCTGGTATGGACGGTCCGC |
| | | GAGTTAAGCAATGGCCACTTACTGAGGAGAAAATCAAGGCA |
| | | CTCATCGAGATCTGCACCGAAATGGAGAAGGAGGGCAAAA |
| | | TAAGCAGGATTGGTCCCGAGAATCCATATAATACGCCGATC |
| | | TTCGCGATAAAAAAGAAGGACGGCACCAAATGGCGAAAAC |
| | | TGGTTGACTTCCGGGAGCTTAACAAAAAAACTCAGGATTTT |
| | | TGGGAAGTTCAACTCGGGATCCCACACCCGTCTGGTCTTAA |
| | | AAAAAAAAAAAGCGTAACAGTCCTTGACATCGGCGATGCCT |
| | | ACTTTAGCGTGCCTCTCGATAAGGAGTTCAGAAAATACACG |
| | | GCTTTCACTGTACCAAGCACAAACAATGAGACTCCTGGGGT |
| | | CAGATACCAGTACAATGTCCTTCCCATGGGGTGGAAAGGAA |
| | | GCCCCGCAATATTCCAGTGCTCAATGACGTGAtagttcgaacggccgt |
| | | atcacgcccaaacatttacagccgcggtgtcaaaaaccgcgtggacgtggttaacatccctgctgggag |
| | | gatcagccgtaattattataattggcttggtgctggctactattgtggccatgtacgtgctgaccaaccagaa |
| | | acataattgaatacagcagcaattggcaagctgcttacatagaactcgcggcgattggcatgccgccttaa |
| | | 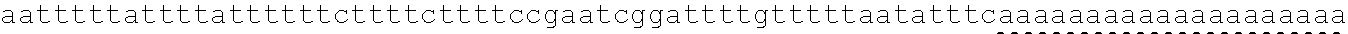 |
| | | 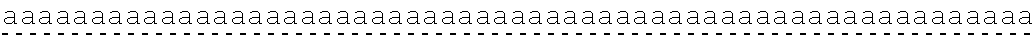 |
| | | 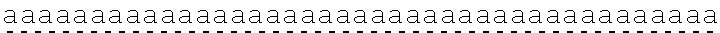 |

TABLE 13
HIV Immunogen Sequences

| SEQ ID | | |
|---|---|---|
| NO: | Description | Sequence |
| 524 | Immunogen | atgatttctcctcggacgctgaatgcatgggtgaaggtagtggag |
| | 1A | gaaaaggcattttcaccagaagtcattccgatgttctccgcgctc |
| | | tccgagggtgctacgccacaggacttgaatacgatgctgaatacc |
| | | gttggtggccatcaggcggcgatgcagatgttgaaggagacaatt |
| | | aacgaagaagccgccgaatgggacagattgcacccggtgcatgcg |
| | | gggccaatagctcctggccagatgcgcgagcctaggggttctgac |
| | | attgctggtacaacaagtacccttcaggagcagattggttggatg |
| | | acgaataaccctcccatacctgttggcgagatctataagcgctgg |
| | | attatacttgggctgaataagatagtccgaatgtattctcccacc |
| | | tctattctggatattcggcaaggacctaaggagccgtttagagac |
| | | tacgtagaccggttttacaaaaccctgcgggcggaacaagcttct |
| | | caggaagtcaaaaattggatgactgagaccttgctcgtccagaat |
| | | gcgaacccggactgtaaaacaatactcaaagcgctgggccccgct |
| | | gcaaccctggaagaaatgatgacggcttgtcagggagtaggaggc |
| | | cccggacataaggcacgagtgttggcagaagccatgagccagccg |
| | | cctgtcgtcgcgaaagaaatcgtcgcttcttgcgacaaatgtcag |
| | | ctgaagggggaggcgatgcacggtcaagttgattgctctcccggt |
| | | atttggcaattggactgtacccaccttgaaggcaaaattattctg |
| | | gttgcagtgcacgtagcatccggttacatcgaagctgaagtgata |
| | | cccgcagagacaggccaggagacggcttatttcctccttaagctt |
| | | gcgggtcggtggcccgtaaagaccatggctgctcgggcatctgtc |
| | | ctctccggtggtgaactcgaccgatgggaaaagattcgattgcgc |
| | | cccggaggaaagaagaaatataggctgaaacatattgtgtgggca |
| | | tcacgggaacttgagcgatttgcggtaaacccaggccttttggaa |
| | | acagctgcaactgtgaaagcggcttgctggtgggcggggataaaa |
| | | caggagtttggtatcccgtacaatccccaatctcagggggtagta |
| | | gaaagcatgaacaaagaattgaaaaaaataattggccaggttcgc |
| | | gaccaagccgagcacctcaagaccgctgtacagatggctgtattt |
| | | attcacaacttcaagcggaagggcggaataggaggatatagcgca |
| | | ggggaaaggattgttgatattattgcaacacaaggttactttcct |
| | | gactggcaaaactacacaccgggccctggcacgcgctatcccctt |
| | | acgttcggttggtgcttcaagctggtgccggtaaaagaaaaagtt |
| | | tatttggcatgggttcctgcacataaaggaatagggggtaacgaa |
| | | caagttgacaaactcgtcagccgcgctaaaagagccccagtcaag |
| | | cagaccctgaattttgacctgcttaaattggctggggacgtcgag |
| | | agtaacccgggacccttcccacaaattacactctggcagcgacca |
| | | ctggtaacaatcaaaatagggggacaattgaaagaagcactcctg |
| | | gatacgggcgcggacgatacagtcctggaggaaatgaatctcccc |
| | | ggccgctggaaacctaagatgataggggggatcggtggatttatt |
| | | aaagtgcggcagtacgatcaatggggttttacgacacctgacaag |
| | | aaacatcaaaaggagccgccatttctttggatgggttatgagctt |
| | | catccggataaatggactgttcagccgattgtcctccccgagaag |
| | | gatagttggactgtgaacgacatccagaagctggtcgggaaactt |
| | | aattgggccagtcaaatatatccaggtattaaagttgccgcacaa |
| | | gaagaggaggaagtagggttccccgtgaaaccgcaagtccctctc |
| | | cggcccatgacctttaagggcgctctcgacctgtcccatttcctc |
| | | cgcgaaaaggggggtttggaaggcttgatcattaccaagattcaa |
| | | aacttcagggtctattatcgagacagtcgcgatcccctttggaaa |
| | | ggacctgcgaaacttctttggaaaggagaaggagccgtggtaatt |
| | | caggataattctgacataaaggtcgtcccacgccgaaaagcgaag |
| | | attataagagattatggcaagcagatggccggggatgattgtgtc |
| | | gcaagtagacaggatgaagaccctaaattcaagcttccaatccag |
| | | aaggaaacgtgggagacatggtggaccgagtattggcaagctact |
| | | tggatcccagaatgggaatttgtgaacactcccccactcgtaaag |
| | | ctgtggtaccagcttgaaaaagaacctatagtcggggcggagacg |
| | | ttctatgtagacggcgccgctaatcgagagacaaaagcagcaggc |
| | | acggtactggtaggcccgacccctgtcaacatcattggacgaaat |
| | | ctgttgacccagattgggtgtacccttaactttcccatttcacca |
| | | atagagaccgtcccggttaaactgaaaccgggtatggatggtccc |
| | | aaagtaaaacagtggccacttaccgaggagaagattaaggcactc |
| | | gttgaaatatgtacagaaatggaaaaagaggggaaaatctctaaa |
| | | attggccctgaaaatccgtacaacactccggtattcgccataaaa |
| | | aagaaggactctaccaagtggcgcaaactcgtggactttagagaa |
| | | ctgaataaaaggacccaggacttttgggaagtccagctgggtatt |
| | | cctcaccccgctggtctcaagaaaaagaaaagtgtcactgtcttg |
| | | gatgttggagatgcgtacttttcagtacctcttgataaagatttt |
| | | cgaaagtataccgcgtttaccattccctccataaataacgaaaca |
| | | ccggggatcaggtatcaatataacgtgcttccacaaggctggaag |
| | | ggttcaccggctattttccaatcttctatgacgtga |
| 525 | Immunogen | atgttgtctcctagaactctgaatgcttgggtaaaggtgatcgaa |
| | 1B (codon- | gaaaaggcattctcacccgaggttatccctatgtttactgcgttg |
| | optimized) | agcgaaggcgcaacaccccatgatctgaacacaatgctgaataca |
| | | atcggcggacatcaagctgctatgcaaatgcttaaggacaccatc |
| | | aatgaggaggcagccgagtgggatcgcgttcatccagtccacgct |
| | | gggcccgttgcgcctggtcagatgagggacccacgaggatccgac |
| | | atcgcagggagcaccagtacactccaagagcagattgcatggatg |
| | | acgaacaatccaccaatacctgtcggtgacatttacaaaaggtgg |
| | | attattatgggcttgaacaaaattgtacggatgtatagcccggtg |
| | | agcatactggacattaaacagggtccaaaagaaccctttcgagat |
| | | tacgttgacagattttataggacactgagggcggaacaagcgtct |
| | | caagacgttaaaaactggatgacagaaacgctgcttgtacagaat |
| | | tccaacccagactgcaaaactatactgaaagcgctcggtcccggt |
| | | gcgacacttgaggaaatgatgagcgcatgccagggcgtcggaggg |
| | | ccgtctcacaaggcccgcgtgttggcggaagctatgtgccaggtg |
| | | gctaaggagatagtagcatgttgtgataaatgtcagcttaaaggt |
| | | gaagctatacatgggcaggtggattgtagcccgggtgtatggcag |
| | | ctggactgtactcatctggaaggaaaggtaatacttgtcgcagtt |
| | | catgtcgcgagcggatacattgaagctgaaatcattcctacggag |
| | | acgggccaagagacagcttacttcatacttaaacttgctgggcga |
| | | tggccggtgacaactatggcggcgcgcgcttcaattttgagtggt |
| | | ggaaagttggacaagtgggagaagattagactcagacccggaggg |
| | | agaaagaagtataaactgaaacatctggtttgggcttcacgcgaa |
| | | cttgaacggtttgctctcaaccccgggctgctcgaaactgctgct |
| | | gctgtaaaggccgcttgttggtggggggggtaaagcaggaattcg |
| | | gaattccatataacactcaaagccagggagtagtggaatccatga |
| | | ataatgaacttaagaagataattggacagattcgcgatcaggctg |
| | | aacatctcaagacggccgtacaaatggcagtattgattcataact |
| | | ttaaacggaagggcggcataggagagtattctgcgggagaacgca |
| | | taatagatataattgcgactcaggggttctttccggattggcaga |
| | | actatacgccggggccaggcattaggttccccctcacgtttggat |
| | | ggtgtttcaagttggtaccgttgctcattaaaaaagaaaaaatct |
| | | acctggcctgggtcccggcgcacaagggtataggggggaacgagc |
| | | aaattgataagctcgtgtcaagggcgaagcgcgcgccagtcaaac |
| | | agaccttgaatttcgacctccttaagctcgctggagacgtcgaat |
| | | ccaaccctggcccgatgctgccacaaatcacattgtggcaacgac |
| | | ccattgtaacaataaaaatcgggggccagatcaaagaagcgctgc |
| | | ttgacaccggcgccgacgatacagtcctcgaggatatgaatttgc |
| | | caggcaaatggaagccgaagatgattggcggcattggcggcttta |
| | | ttaaggttaaacagtatgatcagtggggattgaccactcccgata |
| | | agaagcatcagaaagatccgccttttctgtggatggggtacgaac |
| | | tgcaccctgatcgatggacggtccagccgatagagctgccggaaa |
| | | aagaatcatggaccgtgaatgatattcaaaaactgatcggaaaac |
| | | tcaattgggcgtcccagatatatgctggcatcaaagttgcagcac |
| | | aagaagaggaagaggtaggtttcccggttcggccgcaagttccct |
| | | tgcgaccgatgacatacaagggcgcattggacctttctcacttcc |
| | | tcaaggaaaagggcggtttggagggcatcactaaacttcagaatt |
| | | tcagagtctactatagagataacagggacccattgtggaagggcc |
| | | ccgctcgacttctctggaaaggggagggagcggttgtaattcaag |
| | | acaacagtgaaattaaggtcgtcccacgacggaaggttaaaataa |
| | | ttcgcgactatggcaagcgaatggcgggagacgactgtgtagcag |
| | | gacgacaagacgaggacccaaagtttagattgccgatccagaaag |
| | | agacatgggatacgtggtggacggactattggcaggccacctgga |
| | | taccagaatgggagtttacaaacactcctccactcgtgaaattgt |
| | | ggtatcaacttgagaccgagcccatagctggtgtagagacgtttt |
| | | acgttgacggtgctagcaacagggaaacaaaggccgctggaaccg |
| | | tgctcatcggtcctactcctgtgaacataattggacgaaatttgt |
| | | tgacccagctgggatgcaccctcaatttccccattagcccaatag |
| | | ataccgtaccagtcaagcttaagcctggtatggacggtccgcgag |
| | | ttaagcaatggccacttactgaggagaaaatcaaggcactcatcg |
| | | agatctgcaccgaaatggagaaggagggcaaaataagcaggattg |
| | | gtcccgagaatccatataatacgccgatcttcgcgataaaaaaga |
| | | aggacggcaccaaatggcgaaaactggttgacttccgggagctta |
| | | acaaaaaaactcaggatttttgggaagttcaactcgggatcccac |
| | | acccgtctggtcttaaaaaaaaaaaaagcgtaacagtccttgaca |
| | | tcggcgatgcctactttagcgtgcctctcgataaggagttcagaa |
| | | aatacacggctttcactgtaccaagcacaaacaatgagactcctg |
| | | gggtcagataccagtacaatgtccttcccatggggtggaaaggaa |
| | | gccccgcaatattccagtgctcaatgacgtga |
| 526 | Immunogen | atgctttcaccgagaaccttgaacgcgtgggttaaagtaatcgaa |
| | 1B | gagaaggcatttagcccggaggtaatccccatgtttaccgcactt |
| | | tccgaaggtgctacaccccacgacctgaatacaatgttgaatact |
| | | attggcggccaccaagctgccatgcagatgcttaaggacactatt |
| | | aacgaggaagccgccgaatgggatcgcgttcaccctgttcacgcg |
| | | ggtccagtcgcaccaggacaaatgcgagaccctcgggggtctgac |
| | | atcgcaggatcaactagtacattgcaggaacagattgcttggatg |
| | | accaataaccctccgatcccggtaggtgacatttataaacggtgg |
| | | atcatcatgggattgaacaaaatagtccgaatgtacagtccagtg |
| | | agtattcttgacattaaacagggaccaaaggagccgttccgggac |
| | | tacgttgaccggttttacaggaccctgagagccgaacaggcttcc |
| | | caggatgtgaaaaactggatgacagagaccctgcttgttcagaat |
| | | tcaaatcctgactgtaaaacgattctgaaggcactcggtcccggc |
| | | gccacgctggaggaaatgatgtcagcttgtcagggagtaggagga |
| | | ccttctcataaggcacgcgtccttgcagaggccatgtgccaagtc |
| | | gcgaaagagatcgtggcctgttgtgacaagtgccaacttaaggga |
| | | gaagccatccatgggcaggtcgattgttctccgggcgtttggcaa |
| | | ttggactgcacgcacttggagggtaaggtgattcttgttgccgtt |
| | | catgtcgcaagcggttacattgaagcggagattatcccaactgag |
| | | accggccaagagactgcatatttcatccttaaactggcgggaaga |
| | | tggccggtcacgactatggctgcacgagcatccatactgtctggg |
| | | ggaaagctcgacaagtgggagaagattcgacttaggcctggagga |
| | | agaaaaaagtataagttgaaacatcttgtttgggcatcacgagaa |
| | | cttgaacggtttgctctgaatccgggattgcttgaaaccgccgcg |
| | | gctgtaaaggcagcatgttggtgggctggtgtcaaacaagagttc |
| | | ggtattccctataacacccaaagtcaaggcgtcgtagaatccatg |
| | | aacaatgaacttaagaaaattatcggacaaatccgggaccaagcg |
| | | gaacacctcaaaactgctgtccagatggcagttctgattcataac |
| | | tttaaaagaaagggggggataggggagtattctgcaggtgaacga |
| | | attatagacataattgccacacaagggttttttccagattggcaa |
| | | aattatacaccgggccctgggataagattcccgctcaccttcggc |
| | | tggtgcttcaaactggtacccttgctgatcaagaaagagaagatc |
| | | tatcttgcttgggtgccagcccataaagggatcgggggtaacgaa |
| | | caaatcgataagctggtgtctagagctaaacgggctcccgtaaaa |
| | | cagaccttgaacttcgatctgcttaaattggcaggggacgtggaa |
| | | agcaaccccgggccaatgctgccccagataacactttggcaacgc |
| | | cccatcgtgacaatcaagatcggtggccaaattaaggaagcactc |
| | | ttggacacgggagcagacgacactgtgctggaggatatgaacctg |
| | | ccgggcaagtggaaaccaaagatgatcgggggcattggcgggttc |
| | | ataaaggttaaacagtacgaccaatgggggttgacaacgcctgat |
| | | aagaagcatcaaaaagatcccccatttttgtggatgggttatgaa |
| | | cttcacccggacaggtggaccgttcagccgatagagctcccagaa |
| | | aaggagtcttggacagttaatgacatacagaaacttattggcaaa |
| | | cttaactgggcttcacagatttatgccggcatcaaagtcgccgcc |
| | | caggaagaagaagaggtaggttttcccgtacgacctcaggttcct |
| | | cttcggcctatgacctataagggtgcgcttgatctttctcacttc |
| | | cttaaagaaaaaggaggtctggaaggtatcacgaaacttcagaat |
| | | tttcgggtgtattaccgggacaacagagacccgctttggaagggg |
| | | ccggctaggcttctgtggaaaggcgagggagcggtagttatccag |
| | | gataactctgagataaaggtagtaccccgacggaaggtaaagatc |
| | | atcagagactacggcaagaggatggctggagacgactgtgtggcc |
| | | gggcgacaggatgaagatcctaaattcaggctgccaatccaaaaa |
| | | gagacgtgggacacatggtggaccgattattggcaggctacgtgg |
| | | atccccgaatgggagtttaccaatactccgccactcgtgaagctt |
| | | tggtaccaattggagacagagcctatagccggcgttgagaccttc |
| | | tacgtggatggggccagcaacagagaaaccaaagcggccggaacg |
| | | gtcctgatcggtcccacacctgttaacatcatagggcgcaatctg |
| | | cttacgcaattggggtgcacattgaattttccaatatcccctatt |
| | | gataccgtgccggttaaattgaagccgggtatggacgggcctcgg |
| | | gtcaagcagtggcccctgaccgaagaaaagatcaaagccctgatt |
| | | gagatctgcacggaaatggaaaaagagggcaagattagtcgcatc |
| | | ggcccggagaacccatacaatactcctatttttgcaattaaaaaa |
| | | aaggacggaacaaagtggaggaaacttgtagatttcagagagctt |
| | | aataagaaaactcaggacttctgggaggtccaactcggtattccg |
| | | catccctccggacttaagaagaaaaagtcagtaaccgtcttggat |
| | | ataggggacgcttatttttcagtgcccctcgataaagaatttcgc |
| | | aaatacacggcgtttactgtgccatctactaataacgaaacgcca |
| | | ggcgtgagatatcaatacaacgtccttcctatgggctggaagggt |
| | | tcacccgcaatttttcagtgctccatgacc |

TABLE 14
HIV Immunogen Sequences

| SEQ ID | | |
|---|---|---|
| NO: | Description | Sequence |
| 527 | Immunogen | MISPRTLNAWVKVVEEKAFSPEVIPMFSALSEGATPQDLNTMLNT |
| | 1A | VGGHQAAMQMLKETINEEAAEWDRLHPVHAGPIAPGQMREPRGS |
| | | DIAGTTSTLQEQIGWMTNNPPIPVGEIYKRWIILGLNKIVRMYSPTS |
| | | ILDIRQGPKEPFRDYVDRFYKTLRAEQASQEVKNWMTETLLVQNA |
| | | NPDCKTILKALGPAATLEEMMTACQGVGGPGHKARVLAEAMSQP |
| | | PVVAKEIVASCDKCQLKGEAMHGQVDCSPGIWQLDCTHLEGKIIL |
| | | VAVHVASGYIEAEVIPAETGQETAYFLLKLAGRWPVKTMAARAS |
| | | VLSGGELDRWEKIRLRPGGKKKYRLKHIVWASRELERFAVNPGLL |
| | | ETAATVKAACWWAGIKQEFGIPYNPQSQGVVESMNKELKKIIGQ |
| | | VRDQAEHLKTAVQMAVFIHNFKRKGGIGGYSAGERIVDIIATQGY |
| | | FPDWQNYTPGPGTRYPLTFGWCFKLVPVKEKVYLAWVPAHKGIG |
| | | GNEQVDKLVSRAKRAPVKQTLNFDLLKLAGDVESNPGPFPQITLW |
| | | QRPLVTIKIGGQLKEALLDTGADDTVLEEMNLPGRWKPKMIGGIG |
| | | GFIKVRQYDQWGFTTPDKKHQKEPPFLWMGYELHPDKWTVQPIV |
| | | LPEKDSWTVNDIQKLVGKLNWASQIYPGIKVAAQEEEEVGFPVKP |
| | | QVPLRPMTFKGALDLSHFLREKGGLEGLIITKIQNFRVYYRDSRDP |
| | | LWKGPAKLLWKGEGAVVIQDNSDIKVVPRRKAKIIRDYGKQMAG |
| | | DDCVASRQDEDPKFKLPIQKETWETWWTEYWQATWIPEWEFVN |
| | | TPPLVKLWYQLEKEPIVGAETFYVDGAANRETKAAGTVLVGPTPV |
| | | NIIGRNLLTQIGCTLNFPISPIETVPVKLKPGMDGPKVKQWPLTEEK |
| | | IKALVEICTEMEKEGKISKIGPENPYNTPVFAIKKKDSTKWRKLVD |
| | | FRELNKRTQDFWEVQLGIPHPAGLKKKKSVTVLDVGDAYFSVPL |
| | | DKDFRKYTAFTIPSINNETPGIRYQYNVLPQGWKGSPAIFQSSMT |
| 528 | Immunogen | MLSPRTLNAWVKVIEEKAFSPEVIPMFTALSEGATPHDLNTMLNT |
| | 1B | IGGHQAAMQMLKDTINEEAAEWDRVHPVHAGPVAPGQMRDPRGSD |
| | | IAGSTSTLQEQIAWMTNNPPIPVGDIYKRWIIMGLNKIVRMYSPV |
| | | SILDIKQGPKEPFRDYVDRFYRTLRAEQASQDVKNWMTETLLVQN |
| | | SNPDCKTILKALGPGATLEEMMSACQGVGGPSHKARVLAEAMCQ |
| | | VAKEIVACCDKCQLKGEAIHGQVDCSPGVWQLDCTHLEGKVILVA |
| | | VHVASGYIEAEIIPTETGQETAYFILKLAGRWPVTTMAARASILS |
| | | GGKLDKWEKIRLRPGGRKKYKLKHLVWASRELERFALNPGLLET |
| | | AAAVKAACWWAGVKQEFGIPYNTQSQGVVESMNNELKKIIGQIRD |
| | | QAEHLKTAVQMAVLIHNFKRKGGIGEYSAGERIIDIIATQGFFPD |
| | | WQNYTPGPGIRFPLTFGWCFKLVPLLIKKEKIYLAWVPAHKGIGG |
| | | NEQIDKLVSRAKRAPVKQTLNFDLLKLAGDVESNPGPMLPQITLW |
| | | QRPIVTIKIGGQIKEALLDTGADDTVLEDMNLPGKWKPKMIGGIG |
| | | GFIKVKQYDQWGLTTPDKKHQKDPPFLWMGYELHPDRWTVQPIE |
| | | LPEKESWTVNDIQKLIGKLNWASQIYAGIKVAAQEEEEVGFPVRP |
| | | QVPLRPMTYKGALDLSHFLKEKGGLEGITKLQNFRVYYRDNRDPL |
| | | WKGPARLLWKGEGAVVIQDNSEIKVVPRRKVKIIRDYGKRMAGD |
| | | DCVAGRQDEDPKFRLPIQKETWDTWWTDYWQATWIPEWEFTNTP |
| | | PLVKLWYQLETEPIAGVETFYVDGASNRETKAAGTVLIGPTPVNI |
| | | IGRNLLTQLGCTLNFPISPIDTVPVKLKPGMDGPRVKQWPLTEEK |
| | | IKALIEICTEMEKEGKISRIGPENPYNTPIFAIKKKDGTKWRKLV |
| | | DFRELNKKTQDFWEVQLGIPHPSGLKKKKSVTVLDIGDAYFSVPL |
| | | DKEFRKYTAFTVPSTNNETPGVRYQYNVLPMGWKGSPAIFQCSMT |

TABLE 15
Promoter Sequences

| SEQ ID | | |
|---|---|---|
| NO: | Description | Sequence |
| 529 | 26s | CTCTCTACGGCTAACCTGAATGGA |
| | subgenomic | |
| | promoter | |
| 530 | CMV | GACATTGATTATTGACTAGTTGTTAATAGTAATCAATTACGGGG |
| | promoter | TCATTAGTTCATAGCCCATATATGGAGTTCCGCGTTACATAACT |
| | | TACGGTAAATGGCCCGCCTGGCTGACCGCCCAACGACCCCCGC |
| | | CCATTGACGTCAATAATGACGTATGTTCCCATAGTAACGCCAAT |
| | | AGGGACTTTCCATTGACGTCAATGGGTGGAGTATTTACGGTAA |
| | | ACTGCCCACTTGGCAGTACATCAAGTGTATCATATGCCAAGTCC |
| | | GCCCCCTATTGACGTCAATGACGGTAAATGGCCCGCCTGGCAT |
| | | TATGCCCAGTACATGACCTTACGGGACTTTCCTACTTGGCAGTA |
| | | CATCTACGTATTAGTCATCGCTATTACCATGGTGATGCGGTTTT |
| | | GGCAGTACACCAATGGGCGTGGATAGCGGTTTGACTCACGGGG |
| | | ATTTCCAAGTCTCCACCCCATTGACGTCAATGGGAGTTTGTTTT |
| | | GGCACCAAAATCAACGGGACTTTCCAAAATGTCGTAATAACCC |
| | | CGCCCCGTTGACGCAAATGGGCGGTAGGCGTGTACGGTGGGAG |
| | | GTCTATATAAGCAGAGCTCGTTTAGTGAACCGTCAG |

TABLE 16
Example 5 Sequences

| SEQ | | |
|---|---|---|
| ID | | |
| NO: | Description | Sequence |
| 531 | Anti-PD1 | QVQLVESGGGVVQPGRSLRLDCKASGITFSNSGMHWVRQAPGKG |
| | antibody | LEWVAVIWYDGSKRYYADSVKGRFTISRDNSKNTLFLQMNSLRA |
| | heavy chain | EDTAVYYCATNDDYWGQGTLVTVSSASTKGPSVFPLASCSRSTSE |
| | | STAALGCLVKDYFPEPVTVSWNSGALTSGVHTFPAVLQSSGLYSL |
| | | SSVVTVPSSSLGTQTYVCNVVHEPSNTKVDKRVEFTPPCPPCPAPE |
| | | LLGGPSVFLFPPKPKDTLMISRTPEVTCVVVDVSQEDPEVQFNWY |
| | | VDGAEVHHAQTKPRERQFNSTYRVVSVLTVTHQDWLNGKEYTC |
| | | KVSNKGLPAPIEKTISKAKGQPREPQVYILPPPQEELTKNQVSLTCL |
| | | VTGFYPSDIAVEWESNGQPENTYKTTPPVLDSDGSYFLYSKLTVD |
| | | KSRWQPGNIFTCSVMHEALHNHYTQKSLSVSPGK |
| 532 | Anti-PD1 | EIVLTQSPATLSLSPGERATLSCRASQSVSSYLAWYQQKPGQ |
| | antibody light | APRLLIYDASNRATGIPARFSGSGSGTDFTLTISSLEPEDFAVY |
| | chain | YCQQSSNWPRTFGQGTKVEIKRAVAAPSVFIFPPSEDQVKSG |
| | | TVSVVCLLNNFYPREASVKWKVDGVLKTGNSQESVTEQDS |
| | | KDNTYSLSSTLTLSSTDYQSHNVYACEVTHQGLSSPVTKSFN |
| | | RGEC |
| 533 | FLT3R-a | TQDCSFQHSPISSDFAVKIRELSDYLLQDYPVTVPSNLQDEEL |
| | | CGALWRLVLAQRWMERLKTVAGSKMQGLLERVNTEIHFVT |
| | | KCAFQHPPSCLRFVQTNISRLLQETSEQLVALKPWITRQNFS |
| | | RCLELQCQPDSSTLPPPRSPGALEATAGGSKPPTCPPCPAPEA |
| | | AGGPSVFLFPPKPKDTLMISRTPEVTCVVVDVSQEDPDVKFN |
| | | WYVNGAEVHHAQTKPRETQYNSTYRVVSVLTVTHQDWLN |
| | | GKEYTCKVSNKALPASIQKTISKDKGQPREPQVYTLPPSREE |
| | | LTKNQVSLTCLVKGFYPSDIVVEWESSGQPENTYKTTPPVLD |
| | | SDGSYFLYSKLTVDKSRWQQGNVFSCSVMHEALHNHYTQK |
| | | SLSVSPGK |
| 534 | ChAd68- | catcatcaataatatacctcaaactttttgtgcgcgttaatatgcaaatgaggcgtttgaatttgggg |
| | CMV- | aggaagggcggtgattggtcgagggatgagcgaccgttaggggcggggcgagtgacgttttg |
| | SIVGag-SV40 | atgacgtggttgcgaggaggagccagtttgcaagttctcgtgggaaaagtgacgtcaaacgag |
| | PolyA | gtgtggtttgaacacggaaatactcaattttcccgcgctctctgacaggaaatgaggtgtttctgg |
| | (uppercase, | gcggatgcaagtgaaaacgggccattttcgcgcgaaaactgaatgaggaagtgaaaatctgag |
| | bold, | taatttcgcgtttatggcagggaggagtatttgccgagggccgagtagactttgaccgattacgtg |
| | underline = | ggggtttcgattaccgtgtttttcacctaaatttccgcgtacggtgtcaaagtccggtgtttttacgta |
| | SIV Gag = nt | ggtgtcagctgatcgccagggtatttaaacctgcgctctccagtcaagaggccactcttgagtgc |
| | 1256-2779; | cagcgagaagagttttctcctccgcgccgcgagtcagatctacactttgaaagtagggataaca |
| | uppercase, | gggtaatGACATTGATTATTGACTAGTTGTTAATAGTAATCAAT |
| | italics, bold, | TACGGGGTCATTAGTTCATAGCCCATATATGGAGTTCCGCG |
| | no underline = | TTACATAACTTACGGTAAATGGCCCGCCTGGCTGACCGCCC |
| | CMV | AACGACCCCCGCCCATTGACGTCAATAATGACGTATGTTCC |
| | promoter = nt | CATAGTAACGCCAATAGGGACTTTCCATTGACGTCAATGGG |
| | 594 to 1195; | TGGAGTATTTACGGTAAACTGCCCACTTGGCAGTACATCAA |
| | uppercase, | GTGTATCATATGCCAAGTCCGCCCCCTATTGACGTCAATGA |
| | bold, italics, | CGGTAAATGGCCCGCCTGGCATTATGCCCAGTACATGACC |
| | dashed | TTACGGGACTTTCCTACTTGGCAGTACATCTACGTATTAGT |
| | underline = | CATCGCTATTACCATGGTGATGCGGTTTTGGCAGTACACCA |
| | poly A = nt | ATGGGCGTGGATAGCGGTTTGACTCACGGGGATTTCCAAG |
| | 2824 to 3047) | TCTCCACCCCATTGACGTCAATGGGAGTTTGTTTTGGCACC |
| | | AAAATCAACGGGACTTTCCAAAATGTCGTAATAACCCCGCC |
| | | CCGTTGACGCAAATGGGCGGTAGGCGTGTACGGTGGGAGG |
| | | TCTATATAAGCAGAGCTCGTTTAGTGAACCGTCAGatcgcctgg |
| | | aacgccatccacgctgttttgacctccatagaagacagcgatcgcgccaccATGGGAG |
| | | CCAGGAACAGTGTGCTCTCAGGCAAGAAGGCAGATGA |
| | | GCTGGAGAAGATCAGGCTGAGACCCAATGGCAAGAAG |
| | | AAGTACATGCTGAAGCATGTGGTCTGGGCAGCCAATG |
| | | AGCTGGACAGGTTTGGCCTGGCAGAGTCCCTGCTGGA |
| | | CAACAAGGAGGGCTGCCAGAAGATCCTGTCAGTGCTG |
| | | GCCCCCCTGGTGCCCACTGGCTCAGAGAACCTGAAGA |
| | | GtCTCTACAACACAGTGTGTGTGATTTGGTGCATCCAT |
| | | GCAGAGGAGAAGGTGAAGCACACTGAGGAGGCCAAG |
| | | CAGATTGTGCAGAGGCACCTGGTGGTGGAGACTGGCA |
| | | CAGCTGACAAGATGCCAGCCACCTCCAGGCCCACAGC |
| | | ACCCCCCTCTGGCAGGGGGGGCAACTACCCAGTCCAG |
| | | CAAGTGGGGGGCAACTATGTGCACCTGCCCCTGAGCC |
| | | CCAGAACCCTGAATGCCTGGGTCAAGCTGGTGGAGGA |
| | | GAAGAAGTTTGGAGCAGAGGTGGTGCCTGGCTTCCAG |
| | | GCCCTGTCAGAGGGATGCACTCCCTATGACATCAACC |
| | | AGATGCTGAACTGTGTGGGAGAGCACCAGGCAGCAAT |
| | | GCAGATCATCAGGGAGATCATCAATGAGGAGGCTGCT |
| | | GACTGGGACCTCCAGCACCCCCAGCCTGGACCCCTCC |
| | | CTGCAGGCCAGCTGAGGGAGCCCAGAGGGAGTGACAT |
| | | AGCAGGCACCACCTCCACAGTGGAGGAGCAGATCCAG |
| | | TGGATGTACAGGCAGCAGAACCCCATCCCTGTGGGCA |
| | | ACATCTACAGGAGGTGGATCCAGCTGGGCCTCCAGAA |
| | | GTGTGTCAGGATGTACAACCCAACCAACATCCTGGAT |
| | | GTGAAGCAGGGCCCCAAGGAGCCCTTCCAGTCTTATG |
| | | TGGACAGGTTCTACAAGAGCCTGAGAGCTGAGCAGAC |
| | | AGACCCTGCTGTGAAGAACTGGATGACCCAGACACTG |
| | | CTGATCCAGAATGCCAATCCTGACTGCAAGCTGGTGC |
| | | TGAAGGGGCTGGGGATGAATCCAACCCTGGAGGAGAT |
| | | GCTGACAGCCTGCCAGGGCATTGGGGGACCTGGACAG |
| | | AAGGCCAGGCTCATGGCAGAGGCTCTCAAGGAGGCCC |
| | | TCAGACCAGACCAGCTGCCATTTGCTGCTGTGCAGCA |
| | | GAAGGGCCAGAGGAGGACCATCAAGTGCTGGAACTGT |
| | | GGCAAGGAGGGCCACTCTGCCAGGCAGTGCAGAGCCC |
| | | CCAGGAGGCAGGGCTGCTGGGGCTGTGGAAAGACAG |
| | | GCCATGTGATGGCCAAGTGCCCAGAGAGGCAGGCAGG |
| | | CTTCCTGGGCTTTGGCCCCTGGGGCAAGAAGCCAAGA |
| | | AACTTCCCCATGGCCCAGATGCCCCAGGGCCTGACCC |
| | | CCACAGCCCCCCCAGAGGACCCAGCTGTGGACCTGCT |
| | | GAAGAACTACATGAAGATGGGCAGGAAGCAGAGGGA |
| | | GAACAGGGAGAGACCCTACAAGGAGGTGACTGAGGAC |
| | | CTGCTGCACCTGAACTCCCTGTTTGGGGAGGACCAGT |
| | | 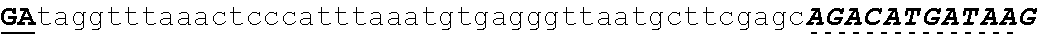 |
| | | 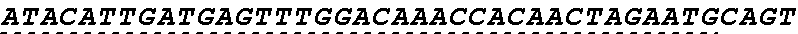 |
| | | 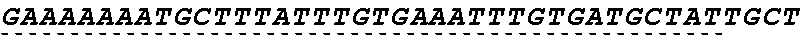 |
| | | 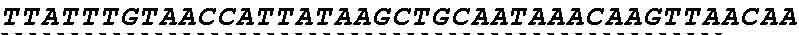 |
| | | 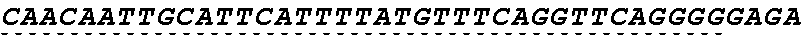 |
| | | 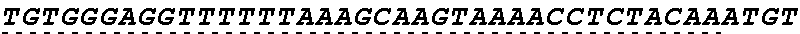 |
| | | 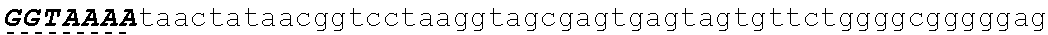 |
| | | gacctgcatgagggccagaataactgaaatctgtgcttttctgtgtgttgcagcagcatgagcgg |
| | | aagcggctcctttgagggaggggtattcagcccttatctgacggggcgtctcccctcctgggcg |
| | | ggagtgcgtcagaatgtgatgggatccacggtggacggccggcccgtgcagcccgcgaactc |
| | | ttcaaccctgacctatgcaaccctgagctcttcgtcgttggacgcagctgccgccgcagctgctg |
| | | catctgccgccagcgccgtgcgcggaatggccatgggcgccggctactacggcactctggtg |
| | | gccaactcgagttccaccaataatcccgccagcctgaacgaggagaagctgttgctgctgatgg |
| | | cccagctcgaggccttgacccagcgcctgggcgagctgacccagcaggtggctcagctgcag |
| | | gagcagacgcgggccgcggttgccacggtgaaatccaaataaaaaatgaatcaataaataaac |
| | | ggagacggttgttgattttaacacagagtctgaatctttatttgatttttcgcgcgcggtaggccctg |
| | | gaccaccggtctcgatcattgagcacccggtggatcttttccaggacccggtagaggtgggctt |
| | | ggatgttgaggtacatgggcatgagcccgtcccgggggtggaggtagctccattgcagggcct |
| | | cgtgctcgggggtggtgttgtaaatcacccagtcatagcaggggcgcagggcatggtgttgca |
| | | caatatctttgaggaggagactgatggccacgggcagccctttggtgtaggtgtttacaaatctgt |
| | | tgagctgggagggatgcatgcggggggagatgaggtgcatcttggcctggatcttgagattgg |
| | | cgatgttaccgcccagatcccgcctggggttcatgttgtgcaggaccaccagcacggtgtatcc |
| | | ggtgcacttggggaatttatcatgcaacttggaagggaaggcgtgaaagaatttggcgacgcct |
| | | ttgtgcccgcccaggttttccatgcactcatccatgatgatggcgatgggcccgtgggcggcgg |
| | | cctgggcaaagacgtttcgggggtcggacacatcatagttgtggtcctgggtgaggtcatcata |
| | | ggccattttaatgaatttggggcggagggtgccggactgggggacaaaggtaccctcgatccc |
| | | gggggcgtagttcccctcacagatctgcatctcccaggctttgagctcggagggggggatcatg |
| | | tccacctgcggggcgataaagaacacggtttccggggcgggggagatgagctgggccgaaa |
| | | gcaagttccggagcagctgggacttgccgcagccggtggggccgtagatgaccccgatgacc |
| | | ggctgcaggtggtagttgagggagagacagctgccgtcctcccggaggaggggggccacct |
| | | cgttcatcatctcgcgcacgtgcatgttctegcgcaccagttccgccaggaggcgctctcccccc |
| | | agggataggagctcctggagcgaggcgaagtttttcagcggcttgagtccgtcggccatgggc |
| | | attttggagagggtttgttgcaagagttccaggcggtcccagagctcggtgatgtgctctacggc |
| | | atctcgatccagcagacctcctcgtttcgcgggttgggacggctgcgggagtagggcaccaga |
| | | cgatgggcgtccagcgcagccagggtccggtccttccagggtcgcagcgtccgcgtcagggt |
| | | ggtctccgtcacggtgaaggggtgcgcgccgggctgggcgcttgcgagggtgcgcttcaggc |
| | | tcatccggctggtcgaaaaccgctcccgatcggcgccctgcgcgtcggccaggtagcaattga |
| | | ccatgagttcgtagttgagcgcctcggccgcgtggcctttggcgcggagcttacctttggaagtc |
| | | tgcccgcaggcgggacagaggagggacttgagggcgtagagcttgggggcgaggaagacg |
| | | gactcgggggcgtaggcgtccgcgccgcagtgggcgcagacggtctcgcactccacgagcc |
| | | aggtgaggtcgggctggtcggggtcaaaaaccagtttcccgccgttctttttgatgcgtttcttacc |
| | | tttggtctccatgagctcgtgtccccgctgggtgacaaagaggctgtccgtgtccccgtagaccg |
| | | actttatgggccggtcctcgagcggtgtgccgcggtcctcctcgtagaggaaccccgcccactc |
| | | cgagacgaaagcccgggtccaggccagcacgaaggaggccacgtgggacgggtagcggtc |
| | | gttgtccaccagcgggtccaccttttccagggtatgcaaacacatgtccccctcgtccacatcca |
| | | ggaaggtgattggcttgtaagtgtaggccacgtgaccgggggtcccggccgggggggtataa |
| | | aagggtgcgggtccctgctcgtcctcactgtcttccggatcgctgtccaggagcgccagctgttg |
| | | gggtaggtattccctctcgaaggcgggcatgacctcggcactcaggttgtcagtttctagaaacg |
| | | aggaggatttgatattgacggtgccggcggagatgcctttcaagagcccctcgtccatctggtca |
| | | gaaaagacgatctttttgttgtcgagcttggtggcgaaggagccgtagagggcgttggagagga |
| | | gcttggcgatggagcgcatggtctggtttttttccttgtcggcgcgctccttggcggcgatgttga |
| | | gctgcacgtactcgcgcgccacgcacttccattcggggaagacggtggtcagctcgtcgggca |
| | | cgattctgacctgccagccccgattatgcagggtgatgaggtccacactggtggccacctegcc |
| | | gcgcaggggctcattagtccagcagaggcgtccgcccttgcgcgagcagaaggggggcagg |
| | | gggtccagcatgacctcgtcgggggggtcggcatcgatggtgaagatgccgggcaggaggt |
| | | cggggtcaaagtagctgatggaagtggccagatcgtccagggcagcttgccattcgcgcacg |
| | | gccagcgcgcgctcgtagggactgaggggcgtgccccagggcatgggatgggtaagcgcg |
| | | gaggcgtacatgccgcagatgtcgtagacgtagaggggctcctcgaggatgccgatgtaggtg |
| | | gggtagcagcgccccccgcggatgctggcgcgcacgtagtcatacagctcgtgcgaggggg |
| | | cgaggagccccgggcccaggttggtgcgactgggcttttcggcgcggtagacgatctggcgg |
| | | aaaatggcatgcgagttggaggagatggtgggcctttggaagatgttgaagtgggcgtggggc |
| | | agtccgaccgagtcgcggatgaagtgggcgtaggagtcttgcagcttggcgacgagctcggc |
| | | ggtgactaggacgtccagagcgcagtagtcgagggtctcctggatgatgtcatacttgagctgt |
| | | cccttttgtttccacagctcgcggttgagaaggaactcttcgcggtccttccagtactcttcgaggg |
| | | ggaacccgtcctgatctgcacggtaagagcctagcatgtagaactggttgacggccttgtaggc |
| | | gcagcagcccttctccacggggagggcgtaggcctgggcggccttgcgcagggaggtgtgc |
| | | gtgagggcgaaagtgtccctgaccatgaccttgaggaactggtgcttgaagtcgatatcgtcgc |
| | | agcccccctgctcccagagctggaagtccgtgcgcttcttgtaggcggggttgggcaaagcga |
| | | aagtaacatcgttgaagaggatcttgcccgcgcggggcataaagttgcgagtgatgcggaaag |
| | | gttggggcacctcggcccggttgttgatgacctgggcggcgagcacgatctcgtcgaagccgt |
| | | tgatgttgtggcccacgatgtagagttccacgaatcgcggacggcccttgacgtggggcagttt |
| | | cttgagctcctcgtaggtgagctcgtcggggtcgctgagcccgtgctgctcgagcgcccagtcg |
| | | gcgagatgggggttggcgcggaggaaggaagtccagagatccacggccagggcggtttgca |
| | | gacggtcccggtactgacggaactgctgcccgacggccattttttcgggggtgacgcagtaga |
| | | aggtgcgggggtccccgtgccagcgatcccatttgagctggagggcgagatcgagggcgag |
| | | ctcgacgagccggtcgtccccggagagtttcatgaccagcatgaaggggacgagctgcttgcc |
| | | gaaggaccccatccaggtgtaggtttccacatcgtaggtgaggaagagcctttcggtgcgagg |
| | | atgcgagccgatggggaagaactggatctcctgccaccaattggaggaatggctgttgatgtga |
| | | tggaagtagaaatgccgacggcgcgccgaacactcgtgcttgtgtttatacaagcggccacagt |
| | | gctcgcaacgctgcacgggatgcacgtgctgcacgagctgtacctgagttcctttgacgaggaa |
| | | tttcagtgggaagtggagtcgtggcgcctgcatctcgtgctgtactacgtcgtggtggtcggcct |
| | | ggccctcttctgcctcgatggtggtcatgctgacgagcccgcgcgggaggcaggtccagacct |
| | | cggcgcgagcgggtcggagagcgaggacgagggcgcgcaggccggagctgtccagggtc |
| | | ctgagacgctgcggagtcaggtcagtgggcagcggcggcgcgcggttgacttgcaggagtttt |
| | | tccagggcgcgcgggaggtccagatggtacttgatctccaccgcgccattggtggcgacgtcg |
| | | atggcttgcagggtcccgtgcccctggggtgtgaccaccgtcccccgtttcttcttgggcggctg |
| | | gggcgacgggggcggtgcctcttccatggttagaagcggcggcgaggacgcgcgccgggc |
| | | ggcaggggcggctcggggcccggaggcaggggcggcaggggcacgtcggcgccgcgcg |
| | | cgggtaggttctggtactgcgcccggagaagactggcgtgagcgacgacgcgacggttgacg |
| | | tcctggatctgacgcctctgggtgaaggccacgggacccgtgagtttgaacctgaaagagagtt |
| | | cgacagaatcaatctcggtatcgttgacggcggcctgccgcaggatctcttgcacgtcgcccga |
| | | gttgtcctggtaggcgatctcggtcatgaactgctcgatctcctcctcttgaaggtctccgcggcc |
| | | ggcgcgctccacggtggccgcgaggtcgttggagatgcggcccatgagctgcgagaaggcg |
| | | ttcatgcccgcctcgttccagacgcggctgtagaccacgacgccctcgggatcgcgggcgcg |
| | | catgaccacctgggcgaggttgagctccacgtggcgcgtgaagaccgcgtagttgcagaggc |
| | | gctggtagaggtagttgagcgtggtggcgatgtgctcggtgacgaagaaatacatgatccagc |
| | | ggcggagcggcatctcgctgacgtcgcccagcgcctccaaacgttccatggcctcgtaaaagt |
| | | ccacggcgaagttgaaaaactgggagttgcgcgccgagacggtcaactcctcctccagaaga |
| | | cggatgagctcggcgatggtggcgcgcacctegcgctcgaaggcccccgggagttcctccac |
| | | ttcctcttcttcctcctccactaacatctcttctacttcctcctcaggcggcagtggtggcggggga |
| | | gggggcctgcgtcgccggcggcgcacgggcagacggtcgatgaagcgctcgatggtctcgc |
| | | cgcgccggcgtcgcatggtctcggtgacggcgcgcccgtcctcgcggggccgcagcgtgaa |
| | | gacgccgccgcgcatctccaggtggccgggggggtccccgttgggcagggagagggcgct |
| | | gacgatgcatcttatcaattgccccgtagggactccgcgcaaggacctgagcgtctcgagatcc |
| | | acgggatctgaaaaccgctgaacgaaggcttcgagccagtcgcagtcgcaaggtaggctgag |
| | | cacggtttcttctggcgggtcatgttggttgggagcggggcgggcgatgctgctggtgatgaag |
| | | ttgaaataggcggttctgagacggcggatggtggcgaggagcaccaggtctttgggcccggct |
| | | tgctggatgcgcagacggtcggccatgccccaggcgtggtcctgacacctggccaggtccttg |
| | | tagtagtcctgcatgagccgctccacgggcacctcctcctcgcccgcgcggccgtgcatgcgc |
| | | gtgagcccgaagccgcgctggggctggacgagcgccaggtcggcgacgacgcgctcggcg |
| | | aggatggcttgctggatctgggtgagggtggtctggaagtcatcaaagtcgacgaagcggtgg |
| | | taggctccggtgttgatggtgtaggagcagttggccatgacggaccagttgacggtctggtggc |
| | | ccggacgcacgagctcgtggtacttgaggcgcgagtaggcgcgcgtgtcgaagatgtagtcgt |
| | | tgcaggtgcgcaccaggtactggtagccgatgaggaagtgcggcggcggctggcggtagag |
| | | cggccatcgctcggtggcgggggcgccgggcgcgaggtcctcgagcatggtgcggtggtag |
| | | ccgtagatgtacctggacatccaggtgatgccggcggcggtggtggaggcgcgcgggaactc |
| | | gcggacgcggttccagatgttgcgcagcggcaggaagtagttcatggtgggcacggtctggc |
| | | ccgtgaggcgcgcgcagtcgtggatgctctatacgggcaaaaacgaaagcggtcagcggctc |
| | | gactccgtggcctggaggctaagcgaacgggttgggctgcgcgtgtaccccggttcgaatctc |
| | | gaatcaggctggagccgcagctaacgtggtattggcactcccgtctcgacccaagcctgcacc |
| | | aaccctccaggatacggaggcgggtcgttttgcaacttttttttggaggccggatgagactagta |
| | | agcgcggaaagcggccgaccgcgatggctcgctgccgtagtctggagaagaatcgccaggg |
| | | ttgcgttgcggtgtgccccggttcgaggccggccggattccgcggctaacgagggcgtggctg |
| | | ccccgtcgtttccaagaccccatagccagccgacttctccagttacggagcgagcccctcttttgt |
| | | tttgtttgtttttgccagatgcatcccgtactgcggcagatgcgcccccaccaccctccaccgcaa |
| | | caacagccccctccacagccggcgcttctgcccccgccccagcagcaacttccagccacgac |
| | | cgccgcggccgccgtgagcggggctggacagagttatgatcaccagctggccttggaagag |
| | | ggcgaggggctggcgcgcctgggggcgtcgtcgccggagcggcacccgcgcgtgcagatg |
| | | aaaagggacgctcgcgaggcctacgtgcccaagcagaacctgttcagagacaggagcggcg |
| | | aggagcccgaggagatgcgcgcggcccggttccacgcggggcgggagctgcggcgcggc |
| | | ctggaccgaaagagggtgctgagggacgaggatttcgaggcggacgagctgacggggatca |
| | | gccccgcgcgcgcgcacgtggccgcggccaacctggtcacggcgtacgagcagaccgtga |
| | | aggaggagagcaacttccaaaaatccttcaacaaccacgtgcgcaccctgatcgcgcgcgag |
| | | gaggtgaccctgggcctgatgcacctgtgggacctgctggaggccatcgtgcagaaccccac |
| | | cagcaagccgctgacggcgcagctgttcctggtggtgcagcatagtcgggacaacgaagcgtt |
| | | cagggaggcgctgctgaatatcaccgagcccgagggccgctggctcctggacctggtgaaca |
| | | ttctgcagagcatcgtggtgcaggagcgcgggctgccgctgtccgagaagctggggccatc |
| | | aacttctcggtgctgagtttgggcaagtactacgctaggaagatctacaagaccccgtacgtgcc |
| | | catagacaaggaggtgaagatcgacgggttttacatgcgcatgaccctgaaagtgctgaccctg |
| | | agcgacgatctgggggtgtaccgcaacgacaggatgcaccgtgcggtgagcgccagcaggc |
| | | ggcgcgagctgagcgaccaggagctgatgcatagtctgcagcgggccctgaccggggccgg |
| | | gaccgagggggagagctactttgacatgggcgcggacctgcactggcagcccagccgccgg |
| | | gccttggaggcggcggcaggaccctacgtagaagaggtggacgatgaggtggacgaggag |
| | | ggcgagtacctggaagactgatggcgcgaccgtatttttgctagatgcaacaacaacagccacc |
| | | tcctgatcccgcgatgcgggcggcgctgcagagccagccgtccggcattaactcctcggacg |
| | | attggacccaggccatgcaacgcatcatggcgctgacgacccgcaaccccgaagcctttagac |
| | | agcagccccaggccaaccggctctcggccatcctggaggccgtggtgccctcgcgctccaac |
| | | cccacgcacgagaaggtcctggccatcgtgaacgcgctggtggagaacaaggccatccgcg |
| | | gcgacgaggccggcctggtgtacaacgcgctgctggagcgcgtggcccgctacaacagcac |
| | | caacgtgcagaccaacctggaccgcatggtgaccgacgtgcgcgaggccgtggcccagcgc |
| | | gagcggttccaccgcgagtccaacctgggatccatggtggcgctgaacgccttcctcagcacc |
| | | cagcccgccaacgtgccccggggccaggaggactacaccaacttcatcagegccctgcgcct |
| | | gatggtgaccgaggtgccccagagcgaggtgtaccagtccgggccggactacttcttccagac |
| | | cagtcgccagggcttgcagaccgtgaacctgagccaggctttcaagaacttgcagggcctgtg |
| | | gggcgtgcaggccccggtcggggaccgcgcgacggtgtcgagcctgctgacgccgaactcg |
| | | cgcctgctgctgctgctggtggcccccttcacggacageggcagcatcaaccgcaactcgtac |
| | | ctgggctacctgattaacctgtaccgcgaggccatcggccaggcgcacgtggacgagcagac |
| | | ctaccaggagatcacccacgtgagccgcgccctgggccaggacgacccgggcaacctggaa |
| | | gccaccctgaactttttgctgaccaaccggtcgcagaagatcccgccccagtacgcgctcagca |
| | | ccgaggaggagcgcatcctgcgttacgtgcagcagagcgtgggcctgttcctgatgcaggag |
| | | ggggccacccccagcgccgcgctcgacatgaccgcgcgcaacatggagcccagcatgtacg |
| | | ccagcaaccgcccgttcatcaataaactgatggactacttgcatcgggcggccgccatgaactc |
| | | tgactatttcaccaacgccatcctgaatccccactggctcccgccgccggggttctacacgggc |
| | | gagtacgacatgcccgaccccaatgacgggttcctgtgggacgatgtggacagcagcgtgttc |
| | | tccccccgaccgggtgctaacgagcgccccttgtggaagaaggaaggcagcgaccgacgcc |
| | | cgtcctcggcgctgtccggccgcgagggtgctgccgcggcggtgcccgaggccgccagtcc |
| | | tttcccgagcttgcccttctcgctgaacagtatccgcagcagcgagctgggcaggatcacgcgc |
| | | ccgcgcttgctgggcgaagaggagtacttgaatgactcgctgttgagacccgagcgggagaa |
| | | gaacttccccaataacgggatagaaagcctggtggacaagatgagccgctggaagacgtatgc |
| | | gcaggagcacagggacgatccccgggcgtcgcagggggccacgagccggggcagcgccg |
| | | cccgtaaacgccggtggcacgacaggcagcggggacagatgtgggacgatgaggactccg |
| | | ccgacgacagcagcgtgttggacttggggggagtggtaacccgttcgctcacctgcgccccc |
| | | gtatcgggcgcatgatgtaagagaaaccgaaaataaatgatactcaccaaggccatggcgacc |
| | | agcgtgcgttcgtttcttctctgttgttgttgtatctagtatgatgaggcgtgcgtacccggagggtc |
| | | ctcctccctcgtacgagagcgtgatgcagcaggcgatggcggcggcggcgatgcagccccc |
| | | gctggaggctccttacgtgcccccgcggtacctggcgcctacggaggggcggaacagcattc |
| | | gttactcggagctggcacccttgtacgataccacccggttgtacctggtggacaacaagtcggc |
| | | ggacatcgcctcgctgaactaccagaacgaccacagcaacttcctgaccaccgtggtgcagaa |
| | | caatgacttcacccccacggaggccagcacccagaccatcaactttgacgagcgctcgcggtg |
| | | gggcggccagctgaaaaccatcatgcacaccaacatgcccaacgtgaacgagttcatgtacag |
| | | caacaagttcaaggcgcgggtgatggtctcccgcaagacccccaatggggtgacagtgacag |
| | | aggattatgatggtagtcaggatgagctgaagtatgaatgggtggaatttgagctgcccgaagg |
| | | caacttctcggtgaccatgaccatcgacctgatgaacaacgccatcatcgacaattacttggcgg |
| | | tggggcggcagaacggggtgctggagagcgacatcggcgtgaagttcgacactaggaacttc |
| | | aggctgggctgggaccccgtgaccgagctggtcatgcccggggtgtacaccaacgaggcttt |
| | | ccatcccgatattgtcttgctgcccggctgcggggtggacttcaccgagagccgcctcagcaac |
| | | ctgctgggcattcgcaagaggcagcccttccaggaaggcttccagatcatgtacgaggatctgg |
| | | aggggggcaacatccccgcgctcctggatgtcgacgcctatgagaaaagcaaggaggatgca |
| | | gcagctgaagcaactgcagccgtagctaccgcctctaccgaggtcaggggcgataattttgca |
| | | agcgccgcagcagtggcagcggccgaggcggctgaaaccgaaagtaagatagtcattcagc |
| | | cggtggagaaggatagcaagaacaggagctacaacgtactaccggacaagataaacaccgc |
| | | ctaccgcagctggtacctagcctacaactatggcgaccccgagaagggcgtgcgctcctggac |
| | | gctgctcaccacctcggacgtcacctgcggcgtggagcaagtctactggtcgctgcccgacat |
| | | gatgcaagacccggtcaccttccgctccacgcgtcaagttagcaactacccggtggtgggcgc |
| | | cgagctcctgcccgtctactccaagagcttcttcaacgagcaggccgtctactcgcagcagctg |
| | | cgcgccttcacctcgcttacgcacgtcttcaaccgcttccccgagaaccagatcctcgtccgccc |
| | | gcccgcgcccaccattaccaccgtcagtgaaaacgttcctgctctcacagatcacgggaccctg |
| | | ccgctgcgcagcagtatccggggagtccagcgcgtgaccgttactgacgccagacgccgcac |
| | | ctgcccctacgtctacaaggccctgggcatagtcgcgccgcgcgtcctctcgagccgcaccttc |
| | | taaatgtccattctcatctcgcccagtaataacaccggttggggcctgcgcgcgcccagcaagat |
| | | gtacggaggcgctcgccaacgctccacgcaacaccccgtgcgcgtgcgcgggcacttccgc |
| | | gctccctggggcgccctcaagggccgcgtgcggtcgcgcaccaccgtcgacgacgtgatcg |
| | | accaggtggtggccgacgcgcgcaactacacccccgccgccgcgcccgtctccaccgtgga |
| | | cgccgtcatcgacagcgtggtggccgacgcgcgccggtacgcccgcgccaagagccggcg |
| | | gcggcgcatcgcccggcggcaccggagcacccccgccatgcgcgcggcgcgagccttgct |
| | | gcgcagggccaggcgcacgggacgcagggccatgctcagggcggccagacgcgcggctt |
| | | caggcgccagcgccggcaggacccggagacgcgcggccacggcggcggcagcggccat |
| | | cgccagcatgtcccgcccgcggcgagggaacgtgtactgggtgcgcgacgccgccaccggt |
| | | gtgcgcgtgcccgtgcgcacccgcccccctcgcacttgaagatgttcacttcgcgatgttgatgt |
| | | gtcccagcggcgaggaggatgtccaagcgcaaattcaaggaagagatgctccaggtcatcgc |
| | | gcctgagatctacggccctgcggtggtgaaggaggaaagaaagccccgcaaaatcaagcgg |
| | | gtcaaaaaggacaaaaaggaagaagaaagtgatgtggacggattggtggagtttgtgcgcga |
| | | gttcgccccccggcggcgcgtgcagtggcgcgggcggaaggtgcaaccggtgctgagaccc |
| | | ggcaccaccgtggtcttcacgcccggcgagcgctccggcaccgcttccaagcgctcctacga |
| | | cgaggtgtacggggatgatgatattctggagcaggcggccgagcgcctgggcgagtttgctta |
| | | cggcaagcgcagccgttccgcaccgaaggaagaggcggtgtccatcccgctggaccacggc |
| | | aaccccacgccgagcctcaagcccgtgaccttgcagcaggtgctgccgaccgcggcgccgc |
| | | gccgggggttcaagcgcgagggcgaggatctgtaccccaccatgcagctgatggtgcccaag |
| | | cgccagaagctggaagacgtgctggagaccatgaaggtggacccggacgtgcagcccgag |
| | | gtcaaggtgcggcccatcaagcaggtggccccgggcctgggcgtgcagaccgtggacatca |
| | | agattcccacggagcccatggaaacgcagaccgagcccatgatcaagcccagcaccagcac |
| | | catggaggtgcagacggatccctggatgccatcggctcctagtcgaagaccccggcgcaagt |
| | | acggcgcggccagcctgctgatgcccaactacgcgctgcatccttccatcatccccacgccgg |
| | | gctaccgcggcacgcgcttctaccgcggtcataccagcagccgccgccgcaagaccaccact |
| | | cgccgccgccgtcgccgcaccgccgctgcaaccacccctgccgccctggtgcggagagtgt |
| | | accgccgcggccgcgcacctctgaccctgccgcgegcgcgctaccacccgagcatcgccatt |
| | | taaactttcgcctgctttgcagatcaatggccctcacatgccgccttcgcgttcccattacgggcta |
| | | ccgaggaagaaaaccgcgccgtagaaggctggcggggaacgggatgcgtcgccaccacca |
| | | ccggcggcggcgcgccatcagcaagcggttggggggaggcttcctgccegcgctgatcccc |
| | | atcatcgccgcggcgatcggggcgatccccggcattgcttccgtggcggtgcaggcctctcag |
| | | cgccactgagacacacttggaaacatcttgtaataaaccaatggactctgacgctcctggtcctgt |
| | | gatgtgttttcgtagacagatggaagacatcaatttttcgtccctggctccgcgacacggcacgc |
| | | ggccgttcatgggcacctggagcgacatcggcaccagccaactgaacgggggcgccttcaat |
| | | tggagcagtctctggagcgggcttaagaatttcgggtccacgcttaaaacctatggcagcaagg |
| | | cgtggaacagcaccacagggcaggcgctgagggataagctgaaagagcagaacttccagca |
| | | gaaggtggtcgatgggctcgcctcgggcatcaacggggtggtggacctggccaaccaggcc |
| | | gtgcagcggcagatcaacagccgcctggacccggtgccgcccgccggctccgtggagatgc |
| | | cgcaggtggaggaggagctgcctcccctggacaagcggggcgagaagcgaccccgccccg |
| | | atgcggaggagacgctgctgacgcacacggacgagccgcccccgtacgaggaggcggtga |
| | | aactgggtctgcccaccacgcggcccategcgcccctggccaccggggtgctgaaacccgaa |
| | | aagcccgcgaccctggacttgcctcctccccagccttcccgcccctctacagtggctaagcccc |
| | | tgccgccggtggccgtggcccgcgcgcgacccgggggcaccgcccgccctcatgcgaactg |
| | | gcagagcactctgaacagcatcgtgggtctgggagtgcagagtgtgaagcgccgccgctgct |
| | | attaaacctaccgtagcgcttaacttgcttgtctgtgtgtgtatgtattatgtcgccgccgccgctgt |
| | | ccaccagaaggaggagtgaagaggcgcgtcgccgagttgcaagatggccaccccatcgatg |
| | | ctgccccagtgggcgtacatgcacatcgccggacaggacgcttcggagtacctgagtccgggt |
| | | ctggtgcagtttgcccgcgccacagacacctacttcagtctggggaacaagtttaggaacccca |
| | | cggtggcgcccacgcacgatgtgaccaccgaccgcagccagcggctgacgctgcgcttcgtg |
| | | cccgtggaccgcgaggacaacacctactcgtacaaagtgcgctacacgctggccgtgggcga |
| | | caaccgcgtgctggacatggccagcacctactttgacatccgcggcgtgctggatcggggccc |
| | | tagcttcaaaccctactccggcaccgcctacaacagtctggcccccaagggagcacccaacac |
| | | ttgtcagtggacatataaagccgatggtgaaactgccacagaaaaaacctatacatatggaaatg |
| | | cacccgtgcagggcattaacatcacaaaagatggtattcaacttggaactgacaccgatgatca |
| | | gccaatctacgcagataaaacctatcagcctgaacctcaagtgggtgatgctgaatggcatgac |
| | | atcactggtactgatgaaaagtatggaggcagagctcttaagcctgataccaaaatgaagccttg |
| | | ttatggttcttttgccaagcctactaataaagaaggaggtcaggcaaatgtgaaaacaggaacag |
| | | gcactactaaagaatatgacatagacatggctttctttgacaacagaagtgcggctgctgctggc |
| | | ctagctccagaaattgttttgtatactgaaaatgtggatttggaaactccagatacccatattgtata |
| | | caaagcaggcacagatgacagcagctcttctattaatttgggtcagcaagccatgcccaacaga |
| | | cctaactacattggtttcagagacaactttatcgggctcatgtactacaacagcactggcaatatg |
| | | ggggtgctggccggtcaggcttctcagctgaatgctgtggttgacttgcaagacagaaacaccg |
| | | agctgtcctaccagctcttgcttgactctctgggtgacagaacccggtatttcagtatgtggaatca |
| | | ggcggtggacagctatgatcctgatgtgcgcattattgaaaatcatggtgtggaggatgaacttc |
| | | ccaactattgtttccctctggatgctgttggcagaacagatacttatcagggaattaaggctaatgg |
| | | aactgatcaaaccacatggaccaaagatgacagtgtcaatgatgctaatgagataggcaagggt |
| | | aatccattcgccatggaaatcaacatccaagccaacctgtggaggaacttcctctacgccaacgt |
| | | ggccctgtacctgcccgactcttacaagtacacgccggccaatgttaccctgcccaccaacacc |
| | | aacacctacgattacatgaacggccgggtggtggcgccctcgctggtggactcctacatcaaca |
| | | tcggggcgcgctggtcgctggatcccatggacaacgtgaaccccttcaaccaccaccgcaatg |
| | | cggggctgcgctaccgctccatgctcctgggcaacgggcgctacgtgcccttccacatccagg |
| | | tgccccagaaatttttcgccatcaagagcctcctgctcctgcccgggtcctacacctacgagtgg |
| | | aacttccgcaaggacgtcaacatgatcctgcagagctccctcggcaacgacctgcgcacggac |
| | | ggggcctccatctccttcaccagcatcaacctctacgccaccttcttccccatggcgcacaacac |
| | | ggcctccacgctcgaggccatgctgcgcaacgacaccaacgaccagtccttcaacgactacct |
| | | ctcggcggccaacatgctctaccccatcceggccaacgccaccaacgtgcccatctccatccc |
| | | ctcgcgcaactgggccgccttccgcggctggtccttcacgcgtctcaagaccaaggagacgcc |
| | | ctcgctgggctccgggttcgacccctacttcgtctactegggctccatcccctacctcgacggca |
| | | ccttctacctcaaccacaccttcaagaaggtctccatcaccttcgactcctccgtcagctggcccg |
| | | gcaacgaccggctcctgacgcccaacgagttcgaaatcaagcgcaccgtcgacggcgaggg |
| | | ctacaacgtggcccagtgcaacatgaccaaggactggttcctggtccagatgctggcccactac |
| | | aacatcggctaccagggcttctacgtgcccgagggctacaaggaccgcatgtactccttcttcc |
| | | gcaacttccagcccatgagccgccaggtggtggacgaggtcaactacaaggactaccaggcc |
| | | gtcaccctggcctaccagcacaacaactegggcttcgtcggctacctcgcgcccaccatgcgc |
| | | cagggccagccctaccccgccaactacccctacccgctcateggcaagagcgccgtcaccag |
| | | cgtcacccagaaaaagttcctctgcgacagggtcatgtggcgcatccccttctccagcaacttca |
| | | tgtccatgggcgcgctcaccgaccteggccagaacatgctctatgccaactccgcccacgcgc |
| | | tagacatgaatttcgaagtcgaccccatggatgagtccacccttctctatgttgtcttcgaagtcttc |
| | | gacgtcgtccgagtgcaccagccccaccgeggcgtcategaggccgtctacctgegcacccc |
| | | cttctcggccggtaacgccaccacctaagctcttgcttcttgcaagccatggccgcgggctccg |
| | | gcgagcaggagctcagggccatcatccgcgacctgggctgcgggccctacttcctgggcacc |
| | | ttcgataagcgcttcccgggattcatggccccgcacaagctggcctgcgccatcgtcaacacgg |
| | | ccggccgcgagaccgggggcgagcactggctggccttcgcctggaacccgcgctcgaacac |
| | | ctgctacctcttcgaccccttcgggttctcggacgagcgcctcaagcagatctaccagttcgagt |
| | | acgagggcctgctgcgccgcagcgccctggccaccgaggaccgctgcgtcaccctggaaaa |
| | | gtccacccagaccgtgcagggtccgcgctcggccgcctgcgggctcttctgctgcatgttcctg |
| | | cacgccttcgtgcactggcccgaccgccccatggacaagaaccccaccatgaacttgctgacg |
| | | ggggtgcccaacggcatgctccagtcgccccaggtggaacccaccctgcgccgcaaccagg |
| | | aggcgctctaccgcttcctcaactcccactccgcctactttegctcccaccgegcgcgcatcgag |
| | | aaggccaccgccttcgaccgcatgaatcaagacatgtaaaccgtgtgtgtatgttaaatgtcttta |
| | | ataaacagcactttcatgttacacatgcatctgagatgatttatttagaaatcgaaagggttctgcc |
| | | gggtctcggcatggcccgcgggcagggacacgttgcggaactggtacttggccagccacttg |
| | | aactcggggatcagcagtttgggcagcggggtgtcggggaaggagtcggtccacagcttccg |
| | | cgtcagttgcagggcgcccagcaggtcgggcgcggagatcttgaaatcgcagttgggacccg |
| | | cgttctgcgcgcgggagttgcggtacacggggttgcagcactggaacaccatcagggccggg |
| | | tgcttcacgctcgccagcaccgtcgcgtcggtgatgctctccacgtcgaggtcctcggcgttgg |
| | | ccatcccgaagggggtcatcttgcaggtctgccttcccatgggggcacgcacccgggcttgtg |
| | | gttgcaatcgcagtgcagggggatcagcatcatctgggcctggtcggcgttcatccccgggtac |
| | | atggccttcatgaaagcctccaattgcctgaacgcctgctgggccttggctccctcggtgaagaa |
| | | gaccccgcaggacttgctagagaactggttggtggcgcacccggcgtcgtgcacgcagcagc |
| | | gcgcgtcgttgttggccagctgcaccacgctgcgcccccagcggttctgggtgatcttggcccg |
| | | gtcggggttctccttcagcgcgcgctgcccgttctcgctcgccacatccatctcgatcatgtgctc |
| | | cttctggatcatggtggtcccgtgcaggcaccgcagcttgccctcggcctcggtgcacccgtgc |
| | | agccacagcgcgcacccggtgcactcccagttcttgtgggcgatctgggaatgcgcgtgcacg |
| | | aagccctgcaggaagcggcccatcatggtggtcagggtcttgttgctagtgaaggtcagcgga |
| | | atgccgcggtgctcctcgttgatgtacaggtggcagatgcggcggtacacctcgccctgctcgg |
| | | gcatcagctggaagttggctttcaggtcggtctccacgcggtagcggtccatcagcatagtcatg |
| | | atttccatacccttctcccaggccgagacgatgggcaggctcatagggttcttcaccatcatctta |
| | | gcgctagcagccgcggccagggggtcgctctcgtccagggtctcaaagctccgcttgccgtcc |
| | | ttctcggtgatccgcaccggggggtagctgaagcccacggccgccagctcctcctcggcctgt |
| | | ctttcgtcctcgctgtcctggctgacgtcctgcaggaccacatgcttggtcttgcggggtttcttctt |
| | | gggcggcagcggcggcggagatgttggagatggcgagggggagcgcgagttctcgctcac |
| | | cactactatctcttcctcttcttggtccgaggccacgcggcggtaggtatgtctcttcgggggcag |
| | | aggcggaggcgacgggctctcgccgccgcgacttggcggatggctggcagagccccttccg |
| | | cgttcgggggtgcgctcccggcggcgctctgactgacttcctccgcggccggccattgtgttct |
| | | cctagggaggaacaacaagcatggagactcagccategccaacctcgccatctgcccccacc |
| | | gccgacgagaagcagcagcagcagaatgaaagcttaaccgccccgccgcccagccccgcc |
| | | acctccgacgcggccgtcccagacatgcaagagatggaggaatccatcgagattgacctggg |
| | | ctatgtgacgcccgcggagcacgaggaggagctggcagtgcgcttttcacaagaagagatac |
| | | accaagaacagccagagcaggaagcagagaatgagcagagtcaggctgggctcgagcatg |
| | | acggcgactacctccacctgagcgggggggaggacgcgctcatcaagcatctggcccggca |
| | | ggccaccatcgtcaaggatgcgctgctcgaccgcaccgaggtgcccctcagcgtggaggagc |
| | | tcagccgcgcctacgagttgaacctcttctcgccgegegtgccccccaagcgccagcccaatg |
| | | gcacctgcgagcccaacccgcgcctcaacttctacccggtcttcgcggtgcccgaggccctgg |
| | | ccacctaccacatctttttcaagaaccaaaagatccccgtctcctgccgcgccaaccgcacccg |
| | | cgccgacgcccttttcaacctgggtcceggcgcccgcctacctgatatcgcctccttggaagag |
| | | gttcccaagatcttcgagggtctgggcagcgacgagactcgggccgcgaacgctctgcaagg |
| | | agaaggaggagagcatgagcaccacagcgccctggtcgagttggaaggcgacaacgcgcg |
| | | gctggcggtgctcaaacgcacggtcgagctgacccatttcgcctacccggctctgaacctgccc |
| | | cccaaagtcatgagcgcggtcatggaccaggtgctcatcaagcgcgcgtcgcccatctccgag |
| | | gacgagggcatgcaagactccgaggagggcaagcccgtggtcagcgacgagcagctggcc |
| | | cggtggctgggtcctaatgctagtccccagagtttggaagagcggcgcaaactcatgatggcc |
| | | gtggtcctggtgaccgtggagctggagtgcctgcgccgcttcttcgccgacgcggagaccctg |
| | | cgcaaggtcgaggagaacctgcactacctcttcaggcacgggttcgtgcgccaggcctgcaa |
| | | gatctccaacgtggagctgaccaacctggtctcctacatgggcatcttgcacgagaaccgcctg |
| | | gggcagaacgtgctgcacaccaccctgcgcggggaggcccggegcgactacatccgcgact |
| | | gcgtctacctctacctctgccacacctggcagacgggcatgggcgtgtggcagcagtgtctgg |
| | | aggagcagaacctgaaagagctctgcaagctcctgcagaagaacctcaagggtctgtggacc |
| | | gggttcgacgagcgcaccaccgcctcggacctggccgacctcattttccccgagcgcctcagg |
| | | ctgacgctgcgcaacggcctgcccgactttatgagccaaagcatgttgcaaaactttcgctctttc |
| | | atcctcgaacgctccggaatcctgcccgccacctgctcegcgctgccctcggacttcgtgccgc |
| | | tgaccttccgcgagtgccccccgccgctgtggagccactgctacctgctgcgcctggccaacta |
| | | cctggcctaccactcggacgtgatcgaggacgtcagcggcgagggcctgctcgagtgccact |
| | | gccgctgcaacctctgcacgccgcaccgctccctggcctgcaacccccagctgctgagcgag |
| | | acccagatcatcggcaccttcgagttgcaagggcccagcgaaggcgagggttcagccgccaa |
| | | ggggggtctgaaactcaccccggggctgtggacctcggcctacttgcgcaagttcgtgcccga |
| | | ggactaccatcccttcgagatcaggttctacgaggaccaatcccatccgcccaaggccgagct |
| | | gtcggcctgcgtcatcacccagggggcgatcctggcccaattgcaagccatccagaaatcccg |
| | | ccaagaattcttgctgaaaaagggccgcggggtctacctcgacccccagaccggtgaggagct |
| | | caaccccggcttcccccaggatgccccgaggaaacaagaagctgaaagtggagctgccgcc |
| | | cgtggaggatttggaggaagactgggagaacagcagtcaggcagaggaggaggagatgga |
| | | ggaagactgggacagcactcaggcagaggaggacagcctgcaagacagtctggaggaaga |
| | | cgaggaggaggcagaggaggaggtggaagaagcagccgccgccagaccgtcgtcctcgg |
| | | cgggggagaaagcaagcagcacggataccatctccgctccgggtcggggtcccgctcgacc |
| | | acacagtagatgggacgagaccggacgattcccgaaccccaccacccagaccggtaagaag |
| | | gagcggcagggatacaagtcctggcgggggcacaaaaacgccatcgtctcctgcttgcaggc |
| | | ctgcgggggcaacatctccttcacccggcgctacctgctcttccaccgcggggtgaactttccc |
| | | cgcaacatcttgcattactaccgtcacctccacagcccctactacttccaagaagaggcagcag |
| | | cagcagaaaaagaccagcagaaaaccagcagctagaaaatccacagcggcggcagcaggt |
| | | ggactgaggatcgcggcgaacgagccggcgcaaacccgggagctgaggaaccggatctttc |
| | | ccaccctctatgccatcttccagcagagtcgggggcaggagcaggaactgaaagtcaagaac |
| | | cgttctctgcgctcgctcacccgcagttgtctgtatcacaagagcgaagaccaacttcagcgcac |
| | | tctcgaggacgccgaggctctcttcaacaagtactgcgcgctcactcttaaagagtagcccgcg |
| | | cccgcccagtcgcagaaaaaggcgggaattacgtcacctgtgcccttcgccctagcegcctcc |
| | | acccatcatcatgagcaaagagattcccacgccttacatgtggagctaccagccccagatgggc |
| | | ctggccgccggtgccgcccaggactactccacccgcatgaattggctcagcgccgggcccgc |
| | | gatgatctcacgggtgaatgacatccgcgcccaccgaaaccagatactcctagaacagtcagc |
| | | gctcaccgccacgccccgcaatcacctcaatccgcgtaattggcccgccgccctggtgtacca |
| | | ggaaattccccagcccacgaccgtactacttccgcgagacgcccaggccgaagtccagctga |
| | | ctaactcaggtgtccagctggcgggcggcgccaccctgtgtcgtcaccgccccgctcagggta |
| | | taaagcggctggtgatccggggcagaggcacacagctcaacgacgaggtggtgagctcttcg |
| | | ctgggtctgcgacctgacggagtcttccaactcgccggatcggggagatcttccttcacgcctc |
| | | gtcaggccgtcctgactttggagagttcgtcctcgcagccccgctcgggtggcatcggcactct |
| | | ccagttcgtggaggagttcactccctcggtctacttcaaccccttctccggctcccccggccacta |
| | | cccggacgagttcatcccgaacttcgacgccatcagcgagtcggtggacggctacgattgaaa |
| | | ctaatcacccccttatccagtgaaataaagatcatattgatgatgattttacagaaataaaaaataat |
| | | catttgatttgaaataaagatacaatcatattgatgatttgagtttaacaaaaaaataaagaatcactt |
| | | acttgaaatctgataccaggtctctgtccatgttttctgccaacaccacttcactcccctcttcccag |
| | | ctctggtactgcaggccccggcgggctgcaaacttcctccacacgctgaaggggatgtcaaatt |
| | | cctcctgtccctcaatcttcattttatcttctatcagatgtccaaaaagcgcgtccgggtggatgatg |
| | | acttcgaccccgtctacccctacgatgcagacaacgcaccgaccgtgcccttcatcaacccccc |
| | | cttcgtctcttcagatggattccaagagaagcccctgggggtgttgtccctgcgactggccgacc |
| | | ccgtcaccaccaagaacggggaaatcaccctcaagctgggagagggggtggacctcgattcc |
| | | tcgggaaaactcatctccaacacggccaccaaggccgccgcccctctcagtttttccaacaaca |
| | | ccatttcccttaacatggatcaccccttttacactaaagatggaaaattatccttacaagtttctccac |
| | | cattaaatatactgagaacaagcattctaaacacactagctttaggttttggatcaggtttaggactc |
| | | cgtggctctgccttggcagtacagttagtctctccacttacatttgatactgatggaaacataaagc |
| | | ttaccttagacagaggtttgcatgttacaacaggagatgcaattgaaagcaacataagctgggct |
| | | aaaggtttaaaatttgaagatggagccatagcaaccaacattggaaatgggttagagtttggaag |
| | | cagtagtacagaaacaggtgttgatgatgcttacccaatccaagttaaacttggatctggccttag |
| | | ctttgacagtacaggagccataatggctggtaacaaagaagacgataaactcactttgtggacaa |
| | | cacctgatccatcaccaaactgtcaaatactcgcagaaaatgatgcaaaactaacactttgcttga |
| | | ctaaatgtggtagtcaaatactggccactgtgtcagtcttagttgtaggaagtggaaacctaaacc |
| | | ccattactggcaccgtaagcagtgctcaggtgtttctacgttttgatgcaaacggtgttcttttaaca |
| | | gaacattctacactaaaaaaatactgggggtataggcagggagatagcatagatggcactccat |
| | | ataccaatgctgtaggattcatgcccaatttaaaagcttatccaaagtcacaaagttctactactaa |
| | | aaataatatagtagggcaagtatacatgaatggagatgtttcaaaacctatgcttctcactataacc |
| | | ctcaatggtactgatgacagcaacagtacatattcaatgtcattttcatacacctggactaatggaa |
| | | gctatgttggagcaacatttggggctaactcttataccttctcatacategcccaagaatgaacact |
| | | gtatcccaccctgcatgccaacccttcccaccccactctgtggaacaaactctgaaacacaaaat |
| | | aaaataaagttcaagtgttttattgattcaacagttttacaggattcgagcagttatttttcctccaccc |
| | | tcccaggacatggaatacaccaccctctccccccgcacagccttgaacatctgaatgccattggt |
| | | gatggacatgcttttggtctccacgttccacacagtttcagagcgagccagtctcgggtcggtca |
| | | gggagatgaaaccctccgggcactcccgcatctgcacctcacagctcaacagctgaggattgt |
| | | cctcggtggtcgggatcacggttatctggaagaagcagaagagcggcggtgggaatcatagtc |
| | | cgcgaacgggatcggccggtggtgtcgcatcaggccccgcagcagtcgctgccgccgccgc |
| | | tccgtcaagctgctgctcagggggtccgggtccagggactccctcagcatgatgcccacggcc |
| | | ctcagcatcagtcgtctggtgcggcgggcgcagcagcgcatgcggatctcgctcaggtcgctg |
| | | cagtacgtgcaacacagaaccaccaggttgttcaacagtccatagttcaacacgctccagccga |
| | | aactcatcgcgggaaggatgctacccacgtggccgtcgtaccagatcctcaggtaaatcaagtg |
| | | gtgccccctccagaacacgctgcccacgtacatgatctccttgggcatgtggcggttcaccacct |
| | | cccggtaccacatcaccctctggttgaacatgcagccccggatgatcctgcggaaccacaggg |
| | | ccagcaccgccccgcccgccatgcagcgaagagaccccgggtcccggcaatggcaatgga |
| | | ggacccaccgctcgtacccgtggatcatctgggagctgaacaagtctatgttggcacagcaca |
| | | ggcatatgctcatgcatctcttcagcactctcaactcctcgggggtcaaaaccatatcccagggc |
| | | acggggaactcttgcaggacagcgaaccccgcagaacagggcaatcctcgcacagaacttac |
| | | attgtgcatggacagggtatcgcaatcaggcagcaccgggtgatcctccaccagagaagcgc |
| | | gggtctcggtctcctcacagcgtggtaagggggccggccgatacgggtgatggcgggacgc |
| | | ggctgatcgtgttcgcgaccgtgtcatgatgcagttgctttcggacattttcgtacttgctgtagca |
| | | gaacctggtccgggcgctgcacaccgatcgccggcggcggtctcggcgcttggaacgctcgg |
| | | tgttgaaattgtaaaacagccactctctcagaccgtgcagcagatctagggcctcaggagtgatg |
| | | aagatcccatcatgcctgatggctctgatcacatcgaccaccgtggaatgggccagacccagc |
| | | cagatgatgcaattttgttgggtttcggtgacggcgggggagggaagaacaggaagaaccatg |
| | | attaacttttaatccaaacggtctcggagtacttcaaaatgaagatcgcggagatggcacctctcg |
| | | cccccgctgtgttggtggaaaataacagccaggtcaaaggtgatacggttctcgagatgttccac |
| | | ggtggcttccagcaaagcctccacgcgcacatccagaaacaagacaatagcgaaagcggga |
| | | gggttctctaattcctcaatcatcatgttacactcctgcaccatccccagataattttcatttttccagc |
| | | cttgaatgattcgaactagttcctgaggtaaatccaagccagccatgataaagagctcgcgcaga |
| | | gcgccctccaccggcattcttaagcacaccctcataattccaagatattctgctcctggttcacctg |
| | | cagcagattgacaagcggaatatcaaaatctctgccgcgatccctgagctcctccctcagcaata |
| | | actgtaagtactctttcatatcctctccgaaatttttagccataggaccaccaggaataagattagg |
| | | gcaagccacagtacagataaaccgaagtcctccccagtgagcattgccaaatgcaagactgct |
| | | ataagcatgctggctagacccggtgatatcttccagataactggacagaaaatcgcccaggcaa |
| | | tttttaagaaaatcaacaaaagaaaaatcctccaggtggacgtttagagcctcgggaacaacgat |
| | | gaagtaaatgcaagcggtgcgttccagcatggttagttagctgatctgtagaaaaaacaaaaatg |
| | | aacattaaaccatgctagcctggcgaacaggtgggtaaatcgttctctccagcaccaggcaggc |
| | | cacggggtctccggcgcgaccctcgtaaaaattgtcgctatgattgaaaaccatcacagagaga |
| | | cgttcccggtggccggcgtgaatgattcgacaagatgaatacacccccggaacattggcgtcc |
| | | gcgagtgaaaaaaagcgcccgaggaagcaataaggcactacaatgctcagtctcaagtccag |
| | | caaagcgatgccatgcggatgaagcacaaaattctcaggtgcgtacaaaatgtaattactcccct |
| | | cctgcacaggcagcaaagcccccgatccctccaggtacacatacaaagcctcagcgtccatag |
| | | cttaccgagcagcagcacacaacaggcgcaagagtcagagaaaggctgagctctaacctgtc |
| | | cacccgctctctgctcaatatatagcccagatctacactgacgtaaaggccaaagtctaaaaata |
| | | cccgccaaataatcacacacgcccagcacacgcccagaaaccggtgacacactcaaaaaaat |
| | | acgcgcacttcctcaaacgcccaaaactgccgtcatttccgggttcccacgctacgtcatcaaaa |
| | | cacgactttcaaattccgtcgaccgttaaaaacgtcacccgccccgcccctaacggtcgcccgt |
| | | ctctcagccaatcagcgccccgcatccccaaattcaaacacctcatttgcatattaacgcgcaca |
| | | aaaagtttgaggtatattattgatgatg |
| 535 | ChAd68- | catcttcaataatatacctcaaactttttgtgcgcgttaatatgcaaatgaggcgtttgaatttgggg |
| | CMV- | aggaagggcggtgattggtcgagggatgagcgaccgttaggggggggcgagtgacgttttg |
| | SIVEnv-SV40 | atgacgtggttgcgaggaggagccagtttgcaagttctcgtgggaaaagtgacgtcaaacgag |
| | PolyA | gtgtggtttgaacacggaaatactcaattttcccgcgctctctgacaggaaatgaggtgtttctgg |
| | (uppercase, | gcggatgcaagtgaaaacgggccattttcgcgcgaaaactgaatgaggaagtgaaaatctgag |
| | bold, | taatttcgcgtttatggcagggaggagtatttgccgagggccgagtagactttgaccgattacgtg |
| | underline = | ggggtttcgattaccgtgtttttcacctaaatttccgcgtacggtgtcaaagtccggtgtttttacgta |
| | SV40 = nt | ggtgtcagctgatcgccagggtatttaaacctgcgctctccagtcaagaggccactcttgagtgc |
| | 1256 to 3358; | cagcgagaagagttttctcctccgcgccgcgagtcagatctacactttgaaagtagggataaca |
| | uppercase, | gggtaatGACATTGATTATTGACTAGTTGTTAATAGTAATCAAT |
| | italics, bold, | TACGGGGTCATTAGTTCATAGCCCATATATGGAGTTCCGCG |
| | no underline = | TTACATAACTTACGGTAAATGGCCCGCCTGGCTGACCGCCC |
| | CMV | AACGACCCCCGCCCATTGACGTCAATAATGACGTATGTTCC |
| | promoter = nt | CATAGTAACGCCAATAGGGACTTTCCATTGACGTCAATGGG |
| | 594 to 1195; | TGGAGTATTTACGGTAAACTGCCCACTTGGCAGTACATCAA |
| | uppercase, | GTGTATCATATGCCAAGTCCGCCCCCTATTGACGTCAATGA |
| | bold, italics, | CGGTAAATGGCCCGCCTGGCATTATGCCCAGTACATGACC |
| | dashed | TTACGGGACTTTCCTACTTGGCAGTACATCTACGTATTAGT |
| | underline = | CATCGCTATTACCATGGTGATGCGGTTTTGGCAGTACACCA |
| | poly A = nt | ATGGGCGTGGATAGCGGTTTGACTCACGGGGATTTCCAAG |
| | 3406 to 3629) | TCTCCACCCCATTGACGTCAATGGGAGTTTGTTTTGGCACC |
| | | AAAATCAACGGGACTTTCCAAAATGTCGTAATAACCCCGCC |
| | | CCGTTGACGCAAATGGGCGGTAGGCGTGTACGGTGGGAGG |
| | | TCTATATAAGCAGAGCTCGTTTAGTGAACCGTCAGatcgcctgg |
| | | aacgccatccacgctgttttgacctccatagaagacagcgatcgcgccaccATGGGCT |
| | | GCCTGGGCAACCAGCTGCTGATCGCCCTGCTGCTGGT |
| | | GTCCGTGCTGGAAATCTGCTGCGTGCAGTACGTGACC |
| | | GTGTTCTACGGCGTGCCTGCCTGGAAGAACGCCACCA |
| | | TCCCCCTGTTCTGCGCCACCCGGAACCGGGACACCTG |
| | | GGGCACCACACAGTGCCTGCCCGACAACGACGACTAC |
| | | AGCGAGCTGGCCGTGAACATCACCGAGGCCTTCGACG |
| | | CCTGGAACAACACCGTGACCGAGCAGGCCATCGAGGA |
| | | CGTGTGGAACCTGTTCGAGACAAGCATCAAGCCCTGC |
| | | GTGAAGCTGACACCCCTGTGTATCGCCATGCGGTGCA |
| | | ACAAGACCGAGACAGACAGATGGGGACTGACCGGCAG |
| | | GGCTGAGACAACCACCACCGCCAAGAGCACCACCTCT |
| | | ACCACCACCACAACCGTGACCCCCAAGGTGATCAACG |
| | | AGGGCGACAGCTGCATCAAGAACAACAGCTGTGCTGG |
| | | CCTCGAACAGGAACCCATGATCGGCTGCAAGTTCAAC |
| | | ATGACAGGCCTGAAGCGGGACAAGAAGATCGAGTACA |
| | | ACGAGACATGGTACTCTCGGGACCTGATCTGCGAGCA |
| | | GCCTGCCAACGGCAGCGAGAGCAAGTGTTACATGCAG |
| | | CATTGCAACACCAGCGTGATCCAGGAAAGCTGCGACA |
| | | AGCACTACTGGGACGCCATCCGGTTCCGGTACTGCGC |
| | | TCCTCCTGGCTACGCCCTGCTGAGGTGCAACGACAGC |
| | | AACTACAGCGGCTTCGCTCCCAAGTGCAGCAAGGTGG |
| | | TGGTGTCCAGCTGCACCCGGATGATGGAAACCCAGAC |
| | | CAGCACCTGGTTCGGCTTCAACGGCACCAGAGCCGAG |
| | | AACCGGACCTACATCTACTGGCACGGCAACAGCAACC |
| | | GGACCATCATCAGCCTGAACAAGTACTACAACCTGAC |
| | | CATGAAGTGCAGAAGGCCTGGCAATAAGACCGTGCTG |
| | | CCCGTGACCATCATGTCTGGCCTGGTGTTCCACAGCC |
| | | AGCCCATCAACGAGAGGCCCAAGCAGGCCTGGTGCCG |
| | | GTTCGGAGGGAACTGGTCCGAGGCCATCCAGGAAGTG |
| | | AAAGAAACCCTGGTGAAGCACCCTCGGTACACCGGCA |
| | | CCAACGACACCCGGAAGATCAACCTGACCGCTCCTGC |
| | | TGGCGGAGATCCCGAGGTGACCTTCATGTGGACCAAC |
| | | TGCAGAGGCGAGTTCCTGTACTGCAAGATGAACTGGT |
| | | TCCTGAACTGGGTGGAGGACCGGGACCAGAACAGCAA |
| | | CAGGTGGAAGCAGCAGAAGAAGCCCGAGCAGCAGAA |
| | | ACGGAACTACGTGCCCTGCCACATCCGGCAGATCATC |
| | | AACACCTGGCACAAAGTGGGCAAGAACGTGTACCTGC |
| | | CTCCCAGAGAGGGCGACCTGACCTGCAACTCCACCGT |
| | | GACCAGCCTGATCGCCGAGATCGACTGGATCAACAAC |
| | | AACGAAACCAACATCACCATGTCTGCCGAGGTGGCCG |
| | | AGCTGTACAGGCTGGAACTGGGCGACTACAAACTGGT |
| | | GGAGATCACCCCCATCGGCCTGGCTCCCACCGACGTC |
| | | AGACGGTACACCACCACAGGAGCCAGCCGGAACAAGA |
| | | GAGGCGTCTTTGTCCTGGGCTTCCTCGGCTTTCTGGC |
| | | CACAGCAGGCTCTGCAATGGGAGCAGCCAGCCTGACC |
| | | CTGTCTGCCCAGAGCCGGACACTGCTGGCAGGCATTG |
| | | TGCAGCAGCAGCAACAGCTCCTGGACGTGGTGAAGCG |
| | | GCAGCATGAACTCCTGCGACTGACCGTGTGGGGCACC |
| | | AAGAACCTGCAGACACGGGTGACCGCCATCGAGAAGT |
| | | ACCTGAAGGACCAGGCTCAGCTCAACTCTTGGGGCTG |
| | | CGCCTTCCGGCAGGTCTGCCACACCACCGTGCCCTGG |
| | | CCCAACGACTCCCTGGTGCCCAACTGGGACAACATGA |
| | | CCTGGCAGGAATGGGAGGGCAAGGTCGACTTCCTGGA |
| | | AGCCAATATCACCCAGCTGCTGGAAGAGGCCCAGATC |
| | | CAGCAGGAAAAGAACATGTACGAGCTGCAGAAGCTGA |
| | | ACTCCTGGGACATCTTCGGCAATTGGTTCGACCTGAC |
| | | CTCCTGGATTAGATACATCCAGTAAtgataggtttaaactcccatttaa |
| | | 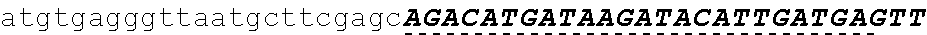 |
| | | 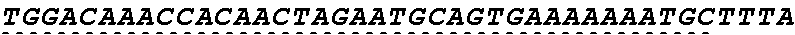 |
| | | 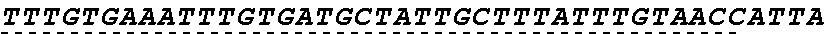 |
| | | 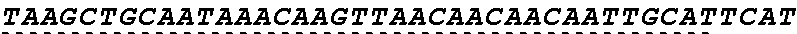 |
| | | 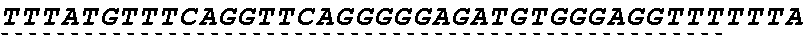 |
| | | 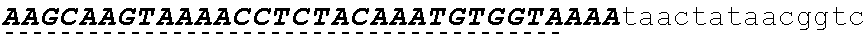 |
| | | ctaaggtagcgagtgagtagtgttctggggcgggggaggacctgcatgagggccagaataac |
| | | tgaaatctgtgcttttctgtgtgttgcagcagcatgagcggaagcggctcctttgagggaggggt |
| | | attcagcccttatctgacggggcgtctcccctcctgggcgggagtgcgtcagaatgtgatggga |
| | | tccacggtggacggccggcccgtgcagcccgcgaactcttcaaccctgacctatgcaaccctg |
| | | agctcttcgtcgttggacgcagctgccgccgcagctgctgcatctgccgccagcgccgtgcgc |
| | | ggaatggccatgggcgccggctactacggcactctggtggccaactcgagttccaccaataat |
| | | cccgccagcctgaacgaggagaagctgttgctgctgatggcccagctcgaggccttgaccca |
| | | gcgcctgggcgagctgacccagcaggtggctcagctgcaggagcagacgcgggccgcggtt |
| | | gccacggtgaaatccaaataaaaaatgaatcaataaataaacggagacggttgttgattttaaca |
| | | cagagtctgaatctttatttgatttttcgcgcgcggtaggccctggaccaccggtctcgatcattga |
| | | gcacccggtggatcttttccaggacccggtagaggtgggcttggatgttgaggtacatgggcat |
| | | gagcccgtcccgggggtggaggtagctccattgcagggcctcgtgctcgggggtggtgttgta |
| | | aatcacccagtcatagcaggggcgcagggcatggtgttgcacaatatctttgaggaggagact |
| | | gatggccacgggcagccctttggtgtaggtgtttacaaatctgttgagctgggagggatgcatgc |
| | | ggggggagatgaggtgcatcttggcctggatcttgagattggcgatgttaccgcccagatcccg |
| | | cctggggttcatgttgtgcaggaccaccagcacggtgtatccggtgcacttggggaatttatcat |
| | | gcaacttggaagggaaggcgtgaaagaatttggcgacgcctttgtgcccgcccaggttttccat |
| | | gcactcatccatgatgatggcgatgggcccgtgggcggcggcctgggcaaagacgtttcggg |
| | | ggtcggacacatcatagttgtggtcctgggtgaggtcatcataggccattttaatgaatttggggc |
| | | ggagggtgccggactgggggacaaaggtaccctcgatcccgggggcgtagttcccctcacag |
| | | atctgcatctcccaggctttgagctcggagggggggatcatgtccacctgcggggcgataaag |
| | | aacacggtttccggggcgggggagatgagctgggccgaaagcaagttccggagcagctggg |
| | | acttgccgcagccggtggggccgtagatgaccccgatgaccggctgcaggtggtagttgagg |
| | | gagagacagctgccgtcctcccggaggaggggggccacctcgttcatcatctcgcgcacgtg |
| | | catgttctcgcgcaccagttccgccaggaggcgctctccccccagggataggagctcctggag |
| | | cgaggcgaagtttttcagcggcttgagtccgtcggccatgggcattttggagagggtttgttgca |
| | | agagttccaggcggtcccagagctcggtgatgtgctctacggcatctcgatccagcagacctcc |
| | | tcgtttcgcgggttgggacggctgcgggagtagggcaccagacgatgggcgtccagcgcagc |
| | | cagggtccggtccttccagggtcgcagcgtccgcgtcagggtggtctccgtcacggtgaaggg |
| | | gtgcgcgccgggctgggcgcttgcgagggtgcgcttcaggctcatccggctggtcgaaaacc |
| | | gctcccgatcggcgccctgcgcgtcggccaggtagcaattgaccatgagttcgtagttgagcg |
| | | cctcggccgcgtggcctttggcgcggagcttacctttggaagtctgcccgcaggcgggacaga |
| | | ggagggacttgagggcgtagagcttgggggcgaggaagacggactcgggggcgtaggcgt |
| | | ccgcgccgcagtgggcgcagacggtctcgcactccacgagccaggtgaggtcgggctggtc |
| | | ggggtcaaaaaccagtttcccgccgttctttttgatgcgtttcttacctttggtctccatgagctcgtg |
| | | tccccgctgggtgacaaagaggctgtccgtgtccccgtagaccgactttatgggccggtcctcg |
| | | agcggtgtgccgcggtcctcctcgtagaggaaccccgcccactccgagacgaaagcccgggt |
| | | ccaggccagcacgaaggaggccacgtgggacgggtagcggtcgttgtccaccagcgggtcc |
| | | accttttccagggtatgcaaacacatgtccccctcgtccacatccaggaaggtgattggcttgtaa |
| | | gtgtaggccacgtgaccgggggtcccggccgggggggtataaaagggtgcgggtccctgct |
| | | cgtcctcactgtcttccggatcgctgtccaggagcgccagctgttggggtaggtattccctctcga |
| | | aggcgggcatgacctcggcactcaggttgtcagtttctagaaacgaggaggatttgatattgacg |
| | | gtgccggcggagatgcctttcaagagcccctcgtccatctggtcagaaaagacgatctttttgttg |
| | | tcgagcttggtggcgaaggagccgtagagggcgttggagaggagcttggcgatggagcgcat |
| | | ggtctggtttttttccttgtcggcgcgctccttggcggcgatgttgagctgcacgtactcgcgcgc |
| | | cacgcacttccattcggggaagacggtggtcagctcgtcgggcacgattctgacctgccagcc |
| | | ccgattatgcagggtgatgaggtccacactggtggccacctcgccgcgcaggggctcattagt |
| | | ccagcagaggcgtccgcccttgcgcgagcagaaggggggcagggggtccagcatgacctc |
| | | gtcgggggggtcggcatcgatggtgaagatgccgggcaggaggtcggggtcaaagtagctg |
| | | atggaagtggccagatcgtccagggcagcttgccattcgcgcacggccagcgcgcgctcgta |
| | | gggactgaggggcgtgccccagggcatgggatgggtaagcgcggaggcgtacatgccgca |
| | | gatgtcgtagacgtagaggggctcctcgaggatgccgatgtaggtggggtagcagegccccc |
| | | cgcggatgctggcgcgcacgtagtcatacagctcgtgcgagggggcgaggagccccgggcc |
| | | caggttggtgcgactgggcttttcggcgcggtagacgatctggcggaaaatggcatgcgagttg |
| | | gaggagatggtgggcctttggaagatgttgaagtgggcgtggggcagtccgaccgagtcgcg |
| | | gatgaagtgggcgtaggagtcttgcagcttggcgacgagctcggcggtgactaggacgtcca |
| | | gagcgcagtagtcgagggtctcctggatgatgtcatacttgagctgtcccttttgtttccacagctc |
| | | gcggttgagaaggaactcttcgcggtccttccagtactcttcgagggggaacccgtcctgatctg |
| | | cacggtaagagcctagcatgtagaactggttgacggccttgtaggcgcagcagcccttctccac |
| | | ggggagggcgtaggcctgggcggccttgcgcagggaggtgtgcgtgagggcgaaagtgtcc |
| | | ctgaccatgaccttgaggaactggtgcttgaagtcgatatcgtcgcagcccccctgctcccaga |
| | | gctggaagtccgtgcgcttcttgtaggcggggttgggcaaagcgaaagtaacatcgttgaaga |
| | | ggatcttgcccgcgcggggcataaagttgcgagtgatgcggaaaggttggggcacctcggcc |
| | | cggttgttgatgacctgggcggcgagcacgatctcgtcgaagccgttgatgttgtggcccacga |
| | | tgtagagttccacgaatcgcggacggcccttgacgtggggcagtttcttgagctcctcgtaggtg |
| | | agctcgtcggggtcgctgagcccgtgctgctcgagcgcccagtcggcgagatgggggttggc |
| | | gcggaggaaggaagtccagagatccacggccagggcggtttgcagacggtcccggtactga |
| | | cggaactgctgcccgacggccattttttcgggggtgacgcagtagaaggtgcgggggtccccg |
| | | tgccagcgatcccatttgagctggagggcgagatcgagggcgagctcgacgagccggtcgtc |
| | | cccggagagtttcatgaccagcatgaaggggacgagctgcttgccgaaggaccccatccagg |
| | | tgtaggtttccacatcgtaggtgaggaagagcctttcggtgcgaggatgcgagccgatgggga |
| | | agaactggatctcctgccaccaattggaggaatggctgttgatgtgatggaagtagaaatgccg |
| | | acggcgcgccgaacactcgtgcttgtgtttatacaagcggccacagtgctcgcaacgctgcac |
| | | gggatgcacgtgctgcacgagctgtacctgagttcctttgacgaggaatttcagtgggaagtgg |
| | | agtcgtggcgcctgcatctcgtgctgtactacgtcgtggtggtcggcctggccctcttctgcctcg |
| | | atggtggtcatgctgacgagcccgcgcgggaggcaggtccagacctcggcgcgagcgggtc |
| | | ggagagcgaggacgagggcgcgcaggccggagctgtccagggtcctgagacgctgcgga |
| | | gtcaggtcagtgggcagcggcggcgcgcggttgacttgcaggagtttttccagggcgcgcgg |
| | | gaggtccagatggtacttgatctccaccgcgccattggtggcgacgtcgatggcttgcagggtc |
| | | ccgtgcccctggggtgtgaccaccgtcccccgtttcttcttgggcggctggggcgacgggggc |
| | | ggtgcctcttccatggttagaagcggcggcgaggacgcgcgccgggcggcaggggggctc |
| | | ggggcccggaggcaggggcggcaggggcacgtcggcgccgcgcgcgggtaggttctggt |
| | | actgcgcccggagaagactggcgtgagcgacgacgcgacggttgacgtcctggatctgacgc |
| | | ctctgggtgaaggccacgggacccgtgagtttgaacctgaaagagagttcgacagaatcaatct |
| | | cggtatcgttgacggcggcctgccgcaggatctcttgcacgtcgcccgagttgtcctggtaggc |
| | | gatctcggtcatgaactgctcgatctcctcctcttgaaggtctccgcggccggcgcgctccacgg |
| | | tggccgcgaggtcgttggagatgcggcccatgagctgcgagaaggcgttcatgcccgcctcgt |
| | | tccagacgcggctgtagaccacgacgccctcgggatcgcgggcgcgcatgaccacctgggc |
| | | gaggttgagctccacgtggcgcgtgaagaccgcgtagttgcagaggcgctggtagaggtagtt |
| | | gagcgtggtggcgatgtgctcggtgacgaagaaatacatgatccagcggcggagcggcatct |
| | | cgctgacgtcgcccagcgcctccaaacgttccatggcctcgtaaaagtccacggcgaagttga |
| | | aaaactgggagttgcgcgccgagacggtcaactcctcctccagaagacggatgagctcggcg |
| | | atggtggcgcgcacctcgcgctcgaaggcccccgggagttcctccacttcctcttcttcctcctc |
| | | cactaacatctcttctacttcctcctcaggcggcagtggtggcgggggagggggcctgcgtcgc |
| | | cggcggcgcacgggcagacggtcgatgaagcgctcgatggtctcgccgcgccggcgtcgca |
| | | tggtctcggtgacggcgcgcccgtcctcgcggggccgcagcgtgaagacgccgccgcgcat |
| | | ctccaggtggccgggggggtccccgttgggcagggagagggcgctgacgatgcatcttatca |
| | | attgccccgtagggactccgcgcaaggacctgagcgtctcgagatccacgggatctgaaaacc |
| | | gctgaacgaaggcttcgagccagtcgcagtcgcaaggtaggctgagcacggtttcttctggcg |
| | | ggtcatgttggttgggagcggggcgggcgatgctgctggtgatgaagttgaaataggcggttct |
| | | gagacggcggatggtggcgaggagcaccaggtctttgggcccggcttgctggatgcgcagac |
| | | ggtcggccatgccccaggcgtggtcctgacacctggccaggtccttgtagtagtcctgcatgag |
| | | ccgctccacgggcacctcctcctcgcccgcgcggccgtgcatgcgcgtgagcccgaagccg |
| | | cgctggggctggacgagcgccaggtcggcgacgacgcgctcggcgaggatggcttgctgga |
| | | tctgggtgagggtggtctggaagtcatcaaagtcgacgaagcggtggtaggctccggtgttgat |
| | | ggtgtaggagcagttggccatgacggaccagttgacggtctggtggcccggacgcacgagct |
| | | cgtggtacttgaggcgcgagtaggcgcgcgtgtcgaagatgtagtcgttgcaggtgcgcacca |
| | | ggtactggtagccgatgaggaagtgcggcggcggctggcggtagagcggccatcgctcggt |
| | | ggcgggggcgccgggcgcgaggtcctcgagcatggtgcggtggtagccgtagatgtacctg |
| | | gacatccaggtgatgccggcggcggtggtggaggcgcgcgggaactcgcggacgcggttcc |
| | | agatgttgcgcagcggcaggaagtagttcatggtgggcacggtctggcccgtgaggcgcgcg |
| | | cagtcgtggatgctctatacgggcaaaaacgaaagcggtcagcggctcgactccgtggcctgg |
| | | aggctaagcgaacgggttgggctgcgcgtgtaccccggttcgaatctcgaatcaggctggagc |
| | | cgcagctaacgtggtattggcactcccgtctcgacccaagcctgcaccaaccctccaggatacg |
| | | gaggcgggtcgttttgcaacttttttttggaggccggatgagactagtaagcgcggaaagcggc |
| | | cgaccgcgatggctcgctgccgtagtctggagaagaatcgccagggttgcgttgcggtgtgcc |
| | | ccggttcgaggccggccggattccgcggctaacgagggcgtggctgccccgtcgtttccaag |
| | | accccatagccagccgacttctccagttacggagcgagcccctcttttgttttgtttgtttttgccag |
| | | atgcatcccgtactgcggcagatgcgcccccaccaccctccaccgcaacaacagccccctcca |
| | | cagccggcgcttctgcccccgccccagcagcaacttccagccacgaccgccgeggccgccg |
| | | tgagcggggctggacagagttatgatcaccagctggccttggaagagggcgaggggctggc |
| | | gcgcctgggggcgtcgtcgccggagcggcacccgcgcgtgcagatgaaaagggacgctcg |
| | | cgaggcctacgtgcccaagcagaacctgttcagagacaggagcggcgaggagcccgagga |
| | | gatgcgcgcggcccggttccacgcgggggggagctgcggcgcggcctggaccgaaagag |
| | | ggtgctgagggacgaggatttcgaggcggacgagctgacggggatcagccccgcgcgcgc |
| | | gcacgtggccgcggccaacctggtcacggcgtacgagcagaccgtgaaggaggagagcaa |
| | | cttccaaaaatccttcaacaaccacgtgcgcaccctgatcgcgcgcgaggaggtgaccctggg |
| | | cctgatgcacctgtgggacctgctggaggccatcgtgcagaaccccaccagcaagccgctga |
| | | cggcgcagctgttcctggtggtgcagcatagtcgggacaacgaagcgttcagggaggcgctg |
| | | ctgaatatcaccgagcccgagggccgctggctcctggacctggtgaacattctgcagagcatc |
| | | gtggtgcaggagcgcgggctgccgctgtccgagaagctggcggccatcaacttctcggtgctg |
| | | agtttgggcaagtactacgctaggaagatctacaagaccccgtacgtgcccatagacaaggag |
| | | gtgaagatcgacgggttttacatgcgcatgaccctgaaagtgctgaccctgagcgacgatctgg |
| | | gggtgtaccgcaacgacaggatgcaccgtgcggtgagcgccagcaggcggcgcgagctga |
| | | gcgaccaggagctgatgcatagtctgcagcgggccctgaccggggccgggaccgaggggg |
| | | agagctactttgacatgggcgcggacctgcactggcagcccagccgccgggccttggaggcg |
| | | gcggcaggaccctacgtagaagaggtggacgatgaggtggacgaggagggcgagtacctg |
| | | gaagactgatggcgcgaccgtatttttgctagatgcaacaacaacagccacctcctgatcccgc |
| | | gatgcgggcggcgctgcagagccagccgtccggcattaactcctcggacgattggacccagg |
| | | ccatgcaacgcatcatggcgctgacgacccgcaaccccgaagcctttagacagcagccccag |
| | | gccaaccggctctcggccatcctggaggccgtggtgccctcgcgctccaaccccacgcacga |
| | | gaaggtcctggccatcgtgaacgcgctggtggagaacaaggccatccgcggcgacgaggcc |
| | | ggcctggtgtacaacgcgctgctggagcgcgtggcccgctacaacagcaccaacgtgcagac |
| | | caacctggaccgcatggtgaccgacgtgcgcgaggccgtggcccagcgcgagcggttccac |
| | | cgcgagtccaacctgggatccatggtggcgctgaacgccttcctcagcacccagcccgccaac |
| | | gtgccccggggccaggaggactacaccaacttcatcagcgccctgcgcctgatggtgaccga |
| | | ggtgccccagagcgaggtgtaccagtccgggccggactacttcttccagaccagtcgccagg |
| | | gcttgcagaccgtgaacctgagccaggctttcaagaacttgcagggcctgtggggcgtgcagg |
| | | ccccggtcggggaccgcgcgacggtgtcgagcctgctgacgccgaactcgcgcctgctgctg |
| | | ctgctggtggcccccttcacggacagcggcagcatcaaccgcaactcgtacctgggctacctg |
| | | attaacctgtaccgcgaggccatcggccaggcgcacgtggacgagcagacctaccaggagat |
| | | cacccacgtgagccgcgccctgggccaggacgacccgggcaacctggaagccaccctgaa |
| | | ctttttgctgaccaaccggtcgcagaagatcccgccccagtacgcgctcagcaccgaggagga |
| | | gcgcatcctgcgttacgtgcagcagagcgtgggcctgttcctgatgcaggagggggccaccc |
| | | ccagcgccgcgctcgacatgaccgcgcgcaacatggagcccagcatgtacgccagcaaccg |
| | | cccgttcatcaataaactgatggactacttgcatcgggcggccgccatgaactctgactatttcac |
| | | caacgccatcctgaatccccactggctcccgccgccggggttctacacgggcgagtacgacat |
| | | gcccgaccccaatgacgggttcctgtgggacgatgtggacagcagcgtgttctccccccgacc |
| | | gggtgctaacgagcgccccttgtggaagaaggaaggcagcgaccgacgcccgtcctcggcg |
| | | ctgtccggccgcgagggtgctgccgcggcggtgcccgaggccgccagtcctttcccgagctt |
| | | gcccttctcgctgaacagtatccgcagcagcgagctgggcaggatcacgcgcccgcgcttgct |
| | | gggcgaagaggagtacttgaatgactcgctgttgagacccgagcgggagaagaacttcccca |
| | | ataacgggatagaaagcctggtggacaagatgagccgctggaagacgtatgcgcaggagca |
| | | cagggacgatccccgggcgtcgcagggggccacgagccggggcagegccgcccgtaaac |
| | | gccggtggcacgacaggcagcggggacagatgtgggacgatgaggactccgccgacgaca |
| | | gcagcgtgttggacttggggggagtggtaacccgttcgctcacctgcgcccccgtatcgggc |
| | | gcatgatgtaagagaaaccgaaaataaatgatactcaccaaggccatggcgaccagcgtgcgt |
| | | tcgtttcttctctgttgttgttgtatctagtatgatgaggcgtgcgtacccggagggtcctcctccctc |
| | | gtacgagagcgtgatgcagcaggcgatggcggcggcggcgatgcagcccccgctggaggc |
| | | tccttacgtgcccccgcggtacctggcgcctacggaggggcggaacagcattcgttactcgga |
| | | gctggcacccttgtacgataccacccggttgtacctggtggacaacaagtcggcggacatcgc |
| | | ctcgctgaactaccagaacgaccacagcaacttcctgaccaccgtggtgcagaacaatgacttc |
| | | acccccacggaggccagcacccagaccatcaactttgacgagcgctcgcggtggggcggcc |
| | | agctgaaaaccatcatgcacaccaacatgcccaacgtgaacgagttcatgtacagcaacaagtt |
| | | caaggcgcgggtgatggtctcccgcaagacccccaatggggtgacagtgacagaggattatg |
| | | atggtagtcaggatgagctgaagtatgaatgggtggaatttgagctgcccgaaggcaacttctc |
| | | ggtgaccatgaccatcgacctgatgaacaacgccatcatcgacaattacttggcggtggggcg |
| | | gcagaacggggtgctggagagcgacatcggcgtgaagttcgacactaggaacttcaggctgg |
| | | gctgggaccccgtgaccgagctggtcatgcccggggtgtacaccaacgaggctttccatcccg |
| | | atattgtcttgctgcccggctgcggggtggacttcaccgagagccgcctcagcaacctgctggg |
| | | cattcgcaagaggcagcccttccaggaaggcttccagatcatgtacgaggatctggagggggg |
| | | caacatccccgcgctcctggatgtcgacgcctatgagaaaagcaaggaggatgcagcagctg |
| | | aagcaactgcagccgtagctaccgcctctaccgaggtcaggggcgataattttgcaagcgccg |
| | | cagcagtggcagcggccgaggcggctgaaaccgaaagtaagatagtcattcagccggtgga |
| | | gaaggatagcaagaacaggagctacaacgtactaccggacaagataaacaccgcctaccgca |
| | | gctggtacctagcctacaactatggcgaccccgagaagggcgtgcgctcctggacgctgctca |
| | | ccacctcggacgtcacctgcggcgtggagcaagtctactggtcgctgcccgacatgatgcaag |
| | | acccggtcaccttccgctccacgcgtcaagttagcaactacccggtggtgggcgccgagctcct |
| | | gcccgtctactccaagagcttcttcaacgagcaggccgtctactcgcagcagctgcgcgccttc |
| | | acctcgcttacgcacgtcttcaaccgettccccgagaaccagatcctcgtccgcccgcccgcgc |
| | | ccaccattaccaccgtcagtgaaaacgttcctgctctcacagatcacgggaccctgccgctgcg |
| | | cagcagtatccggggagtccagcgcgtgaccgttactgacgccagacgccgcacctgcccct |
| | | acgtctacaaggccctgggcatagtcgcgccgcgcgtcctctcgagccgcaccttctaaatgtc |
| | | cattctcatctcgcccagtaataacaccggttggggcctgcgcgcgcccagcaagatgtacgga |
| | | ggcgctcgccaacgctccacgcaacaccccgtgcgcgtgcgcgggcacttccgcgctccctg |
| | | gggcgccctcaagggccgcgtgcggtcgcgcaccaccgtcgacgacgtgatcgaccaggtg |
| | | gtggccgacgcgcgcaactacacccccgccgccgcgcccgtctccaccgtggacgccgtcat |
| | | cgacagcgtggtggccgacgcgcgccggtacgcccgcgccaagagccggcggcggcgcat |
| | | cgcccggcggcaccggagcacccccgccatgcgcgcggcgcgagccttgctgcgcagggc |
| | | caggcgcacgggacgcagggccatgctcagggcggccagacgcgcggcttcaggcgccag |
| | | cgccggcaggacccggagacgcgcggccacggcggcggcagcggccatcgccagcatgt |
| | | cccgcccgcggcgagggaacgtgtactgggtgcgcgacgccgccaccggtgtgcgcgtgcc |
| | | cgtgcgcacccgcccccctcgcacttgaagatgttcacttcgcgatgttgatgtgtcccagcggc |
| | | gaggaggatgtccaagcgcaaattcaaggaagagatgctccaggtcatcgcgcctgagatcta |
| | | cggccctgcggtggtgaaggaggaaagaaagccccgcaaaatcaagcgggtcaaaaagga |
| | | caaaaaggaagaagaaagtgatgtggacggattggtggagtttgtgcgcgagttcgccccccg |
| | | gcggcgcgtgcagtggcgcgggcggaaggtgcaaccggtgctgagacccggcaccaccgt |
| | | ggtcttcacgcccggcgagcgctccggcaccgcttccaagcgctcctacgacgaggtgtacg |
| | | gggatgatgatattctggagcaggcggccgagcgcctgggcgagtttgcttacggcaagcgca |
| | | gccgttccgcaccgaaggaagaggcggtgtccatcccgctggaccacggcaaccccacgcc |
| | | gagcctcaagcccgtgaccttgcagcaggtgctgccgaccgcggcgccgcgccgggggttc |
| | | aagcgcgagggcgaggatctgtaccccaccatgcagctgatggtgcccaagcgccagaagct |
| | | ggaagacgtgctggagaccatgaaggtggacccggacgtgcagcccgaggtcaaggtgcgg |
| | | cccatcaagcaggtggccccgggcctgggcgtgcagaccgtggacatcaagattcccacgga |
| | | gcccatggaaacgcagaccgagcccatgatcaagcccagcaccagcaccatggaggtgcag |
| | | acggatccctggatgccatcggctcctagtcgaagaccccggcgcaagtacggcgcggccag |
| | | cctgctgatgcccaactacgcgctgcatccttccatcatccccacgccgggctaccgcggcacg |
| | | cgcttctaccgcggtcataccagcagccgccgccgcaagaccaccactcgccgccgccgtcg |
| | | ccgcaccgccgctgcaaccacccctgccgccctggtgcggagagtgtaccgccgcggccgc |
| | | gcacctctgaccctgccgegcgcgcgctaccacccgagcategccatttaaactttcgcctgctt |
| | | tgcagatcaatggccctcacatgccgccttcgcgttcccattacgggctaccgaggaagaaaac |
| | | cgcgccgtagaaggctggcggggaacgggatgcgtcgccaccaccaccggcggcggcgc |
| | | gccatcagcaagcggttggggggaggcttcctgcccgcgctgatccccatcatcgccgcggc |
| | | gatcggggcgatccccggcattgcttccgtggcggtgcaggcctctcagcgccactgagacac |
| | | acttggaaacatcttgtaataaaccaatggactctgacgctcctggtcctgtgatgtgttttcgtaga |
| | | cagatggaagacatcaatttttcgtccctggctccgcgacacggcacgcggccgttcatgggca |
| | | cctggagcgacatcggcaccagccaactgaacgggggcgccttcaattggagcagtctctgg |
| | | agcgggcttaagaatttcgggtccacgcttaaaacctatggcagcaaggcgtggaacagcacc |
| | | acagggcaggcgctgagggataagctgaaagagcagaacttccagcagaaggtggtcgatg |
| | | ggctcgcctcgggcatcaacggggtggtggacctggccaaccaggccgtgcagcggcagat |
| | | caacagccgcctggacccggtgccgcccgccggctccgtggagatgccgcaggtggagga |
| | | ggagctgcctcccctggacaagcggggcgagaagcgaccccgccccgatgcggaggagac |
| | | gctgctgacgcacacggacgagccgcccccgtacgaggaggcggtgaaactgggtctgccc |
| | | accacgcggcccategcgcccctggccaccggggtgctgaaacccgaaaagccegcgaccc |
| | | tggacttgcctcctccccagccttcccgcccctctacagtggctaagcccctgccgccggtggc |
| | | cgtggcccgcgcgcgacccgggggcaccgcccgccctcatgcgaactggcagagcactctg |
| | | aacagcatcgtgggtctgggagtgcagagtgtgaagcgccgccgctgctattaaacctaccgta |
| | | gcgcttaacttgcttgtctgtgtgtgtatgtattatgtcgccgccgccgctgtccaccagaaggag |
| | | gagtgaagaggcgcgtcgccgagttgcaagatggccaccccatcgatgctgccccagtgggc |
| | | gtacatgcacatcgccggacaggacgcttcggagtacctgagtccgggtctggtgcagtttgcc |
| | | cgcgccacagacacctacttcagtctggggaacaagtttaggaaccccacggtggcgcccac |
| | | gcacgatgtgaccaccgaccgcagccagcggctgacgctgcgcttcgtgcccgtggaccgcg |
| | | aggacaacacctactcgtacaaagtgcgctacacgctggccgtgggcgacaaccgcgtgctg |
| | | gacatggccagcacctactttgacatccgcggcgtgctggatcggggccctagcttcaaaccct |
| | | actccggcaccgcctacaacagtctggcccccaagggagcacccaacacttgtcagtggacat |
| | | ataaagccgatggtgaaactgccacagaaaaaacctatacatatggaaatgcacccgtgcagg |
| | | gcattaacatcacaaaagatggtattcaacttggaactgacaccgatgatcagccaatctacgca |
| | | gataaaacctatcagcctgaacctcaagtgggtgatgctgaatggcatgacatcactggtactga |
| | | tgaaaagtatggaggcagagctcttaagcctgataccaaaatgaagccttgttatggttcttttgcc |
| | | aagcctactaataaagaaggaggtcaggcaaatgtgaaaacaggaacaggcactactaaaga |
| | | atatgacatagacatggctttctttgacaacagaagtgcggctgctgctggcctagctccagaaat |
| | | tgttttgtatactgaaaatgtggatttggaaactccagatacccatattgtatacaaagcaggcaca |
| | | gatgacagcagctcttctattaatttgggtcagcaagccatgcccaacagacctaactacattggt |
| | | ttcagagacaactttatcgggctcatgtactacaacagcactggcaatatgggggtgctggccg |
| | | gtcaggcttctcagctgaatgctgtggttgacttgcaagacagaaacaccgagctgtcctaccag |
| | | ctcttgcttgactctctgggtgacagaacccggtatttcagtatgtggaatcaggcggtggacag |
| | | ctatgatcctgatgtgcgcattattgaaaatcatggtgtggaggatgaacttcccaactattgtttcc |
| | | ctctggatgctgttggcagaacagatacttatcagggaattaaggctaatggaactgatcaaacc |
| | | acatggaccaaagatgacagtgtcaatgatgctaatgagataggcaagggtaatccattcgcca |
| | | tggaaatcaacatccaagccaacctgtggaggaacttcctctacgccaacgtggccctgtacct |
| | | gcccgactcttacaagtacacgccggccaatgttaccctgcccaccaacaccaacacctacgat |
| | | tacatgaacggccgggtggtggcgccctcgctggtggactcctacatcaacatcggggcgcgc |
| | | tggtcgctggatcccatggacaacgtgaaccccttcaaccaccaccgcaatgcggggctgcgc |
| | | taccgctccatgctcctgggcaacgggcgctacgtgcccttccacatccaggtgccccagaaat |
| | | ttttcgccatcaagagcctcctgctcctgcccgggtcctacacctacgagtggaacttccgcaag |
| | | gacgtcaacatgatcctgcagagctccctcggcaacgacctgcgcacggacggggcctccat |
| | | ctccttcaccagcatcaacctctacgccaccttcttccccatggcgcacaacacggcctccacgc |
| | | tcgaggccatgctgcgcaacgacaccaacgaccagtccttcaacgactacctctcggcggcca |
| | | acatgctctaccccatcccggccaacgccaccaacgtgcccatctccatcccctcgcgcaactg |
| | | ggccgccttccgcggctggtccttcacgcgtctcaagaccaaggagacgccctcgctgggctc |
| | | cgggttcgacccctacttcgtctactegggctccatcccctacctcgacggcaccttctacctcaa |
| | | ccacaccttcaagaaggtctccatcaccttcgactcctccgtcagctggcccggcaacgaccgg |
| | | ctcctgacgcccaacgagttcgaaatcaagcgcaccgtcgacggcgagggctacaacgtggc |
| | | ccagtgcaacatgaccaaggactggttcctggtccagatgctggcccactacaacateggctac |
| | | cagggcttctacgtgcccgagggctacaaggaccgcatgtactccttcttccgcaacttccagcc |
| | | catgagccgccaggtggtggacgaggtcaactacaaggactaccaggccgtcaccctggcct |
| | | accagcacaacaactegggcttcgtcggctacctcgcgcccaccatgcgccagggccagccc |
| | | taccccgccaactacccctacccgctcateggcaagagcgccgtcaccagcgtcacccagaa |
| | | aaagttcctctgcgacagggtcatgtggcgcatccccttctccagcaacttcatgtccatgggcg |
| | | cgctcaccgacctcggccagaacatgctctatgccaactccgcccacgcgctagacatgaattt |
| | | cgaagtcgaccccatggatgagtccacccttctctatgttgtcttcgaagtcttcgacgtcgtccg |
| | | agtgcaccagccccaccgcggcgtcatcgaggccgtctacctgcgcacccccttctcggccg |
| | | gtaacgccaccacctaagctcttgcttcttgcaagccatggccgcgggctccggcgagcagga |
| | | gctcagggccatcatccgcgacctgggctgcgggccctacttcctgggcaccttcgataagcg |
| | | cttcccgggattcatggccccgcacaagctggcctgcgccatcgtcaacacggccggccgcg |
| | | agaccgggggcgagcactggctggccttcgcctggaaccegcgctcgaacacctgctacctct |
| | | tcgaccccttcgggttctcggacgagcgcctcaagcagatctaccagttcgagtacgagggcct |
| | | gctgcgccgcagcgccctggccaccgaggaccgctgcgtcaccctggaaaagtccacccag |
| | | accgtgcagggtccgcgctcggccgcctgcgggctcttctgctgcatgttcctgcacgccttcgt |
| | | gcactggcccgaccgccccatggacaagaaccccaccatgaacttgctgacgggggtgccca |
| | | acggcatgctccagtcgccccaggtggaacccaccctgcgccgcaaccaggaggcgctctac |
| | | cgcttcctcaactcccactccgcctactttcgctcccaccgcgcgcgcatcgagaaggccaccg |
| | | ccttcgaccgcatgaatcaagacatgtaaaccgtgtgtgtatgttaaatgtctttaataaacagcac |
| | | tttcatgttacacatgcatctgagatgatttatttagaaatcgaaagggttctgccgggtctcggcat |
| | | ggcccgcgggcagggacacgttgcggaactggtacttggccagccacttgaactcggggatc |
| | | agcagtttgggcagcggggtgtcggggaaggagtcggtccacagcttccgcgtcagttgcag |
| | | ggcgcccagcaggtcgggcgcggagatcttgaaatcgcagttgggacccgcgttctgcgcgc |
| | | gggagttgcggtacacggggttgcagcactggaacaccatcagggccgggtgcttcacgctc |
| | | gccagcaccgtcgcgtcggtgatgctctccacgtcgaggtcctcggcgttggccatcccgaag |
| | | ggggtcatcttgcaggtctgccttcccatggtgggcacgcacccgggcttgtggttgcaatcgc |
| | | agtgcagggggatcagcatcatctgggcctggtcggcgttcatccccgggtacatggccttcat |
| | | gaaagcctccaattgcctgaacgcctgctgggccttggctccctcggtgaagaagaccccgca |
| | | ggacttgctagagaactggttggtggcgcacccggcgtcgtgcacgcagcagcgcgcgtcgtt |
| | | gttggccagctgcaccacgctgcgcccccagcggttctgggtgatcttggcccggtcggggttc |
| | | tccttcagcgcgcgctgcccgttctcgctcgccacatccatctcgatcatgtgctccttctggatca |
| | | tggtggtcccgtgcaggcaccgcagcttgccctcggcctcggtgcacccgtgcagccacagc |
| | | gcgcacccggtgcactcccagttcttgtgggcgatctgggaatgcgcgtgcacgaagccctgc |
| | | aggaagcggcccatcatggtggtcagggtcttgttgctagtgaaggtcagcggaatgccgcgg |
| | | tgctcctcgttgatgtacaggtggcagatgcggcggtacacctcgccctgctcgggcatcagct |
| | | ggaagttggctttcaggtcggtctccacgcggtagcggtccatcagcatagtcatgatttccatac |
| | | ccttctcccaggccgagacgatgggcaggctcatagggttcttcaccatcatcttagcgctagca |
| | | gccgcggccagggggtcgctctcgtccagggtctcaaagctccgcttgccgtccttctcggtga |
| | | tccgcaccggggggtagctgaagcccacggccgccagctcctccteggcctgtctttegtcctc |
| | | gctgtcctggctgacgtcctgcaggaccacatgcttggtcttgcggggtttcttcttgggcggca |
| | | gcggcggcggagatgttggagatggcgagggggagcgcgagttctcgctcaccactactatct |
| | | cttcctcttcttggtccgaggccacgcggcggtaggtatgtctcttcgggggcagaggcggagg |
| | | cgacgggctctcgccgccgcgacttggcggatggctggcagagccccttccgcgttcggggg |
| | | tgcgctcccggcggcgctctgactgacttcctccgcggccggccattgtgttctcctagggagg |
| | | aacaacaagcatggagactcagccatcgccaacctcgccatctgcccccaccgccgacgaga |
| | | agcagcagcagcagaatgaaagcttaaccgccccgccgcccagccccgccacctccgacgc |
| | | ggccgtcccagacatgcaagagatggaggaatccatcgagattgacctgggctatgtgacgcc |
| | | cgcggagcacgaggaggagctggcagtgcgcttttcacaagaagagatacaccaagaacag |
| | | ccagagcaggaagcagagaatgagcagagtcaggctgggctcgagcatgacggcgactacc |
| | | tccacctgagcgggggggaggacgcgctcatcaagcatctggcccggcaggccaccategtc |
| | | aaggatgcgctgctcgaccgcaccgaggtgcccctcagcgtggaggagctcagccgcgccta |
| | | cgagttgaacctcttctcgccgcgcgtgccccccaagcgccagcccaatggcacctgcgagcc |
| | | caacccgcgcctcaacttctacccggtcttegcggtgcccgaggccctggccacctaccacatc |
| | | tttttcaagaaccaaaagatccccgtctcctgccgcgccaaccgcaccegcgccgacgccctttt |
| | | caacctgggtcccggcgcccgcctacctgatatcgcctccttggaagaggttcccaagatcttc |
| | | gagggtctgggcagcgacgagactcgggccgcgaacgctctgcaaggagaaggaggagag |
| | | catgagcaccacagcgccctggtcgagttggaaggcgacaacgcgcggctggcggtgctca |
| | | aacgcacggtcgagctgacccatttcgcctacccggctctgaacctgccccccaaagtcatgag |
| | | cgcggtcatggaccaggtgctcatcaagcgcgcgtcgcccatctccgaggacgagggcatgc |
| | | aagactccgaggagggcaagcccgtggtcagcgacgagcagctggcccggtggctgggtcc |
| | | taatgctagtccccagagtttggaagagcggcgcaaactcatgatggccgtggtcctggtgacc |
| | | gtggagctggagtgcctgcgccgcttcttcgccgacgcggagaccctgcgcaaggtcgagga |
| | | gaacctgcactacctcttcaggcacgggttcgtgcgccaggcctgcaagatctccaacgtgga |
| | | gctgaccaacctggtctcctacatgggcatcttgcacgagaaccgcctggggcagaacgtgct |
| | | gcacaccaccctgcgcggggaggcccggcgcgactacatccgcgactgcgtctacctctacc |
| | | tctgccacacctggcagacgggcatgggcgtgtggcagcagtgtctggaggagcagaacctg |
| | | aaagagctctgcaagctcctgcagaagaacctcaagggtctgtggaccgggttcgacgagcg |
| | | caccaccgcctcggacctggccgacctcattttccccgagcgcctcaggctgacgctgcgcaa |
| | | cggcctgcccgactttatgagccaaagcatgttgcaaaactttcgctctttcatcctegaacgctc |
| | | cggaatcctgcccgccacctgctccgcgctgccctcggacttcgtgccgctgaccttccgcgag |
| | | tgccccccgccgctgtggagccactgctacctgctgcgcctggccaactacctggcctaccact |
| | | cggacgtgatcgaggacgtcagcggcgagggcctgctcgagtgccactgccgctgcaacctc |
| | | tgcacgccgcaccgctccctggcctgcaacccccagctgctgagcgagacccagatcatcgg |
| | | caccttcgagttgcaagggcccagcgaaggcgagggttcagccgccaaggggggtctgaaa |
| | | ctcaccccggggctgtggacctcggcctacttgcgcaagttcgtgcccgaggactaccatccct |
| | | tcgagatcaggttctacgaggaccaatcccatccgcccaaggccgagctgtcggcctgcgtcat |
| | | cacccagggggcgatcctggcccaattgcaagccatccagaaatcccgccaagaattcttgct |
| | | gaaaaagggccgcggggtctacctcgacccccagaccggtgaggagctcaaccccggcttc |
| | | ccccaggatgccccgaggaaacaagaagctgaaagtggagctgccgcccgtggaggatttg |
| | | gaggaagactgggagaacagcagtcaggcagaggaggaggagatggaggaagactggga |
| | | cagcactcaggcagaggaggacagcctgcaagacagtctggaggaagacgaggaggaggc |
| | | agaggaggaggtggaagaagcagccgccgccagaccgtcgtcctcggcgggggagaaag |
| | | caagcagcacggataccatctccgctccgggtcggggtcccgctcgaccacacagtagatgg |
| | | gacgagaccggacgattcccgaaccccaccacccagaccggtaagaaggagcggcaggga |
| | | tacaagtcctggcgggggcacaaaaacgccatcgtctcctgcttgcaggcctgcgggggcaa |
| | | catctccttcacccggcgctacctgctcttccaccgcggggtgaactttccccgcaacatcttgca |
| | | ttactaccgtcacctccacagcccctactacttccaagaagaggcagcagcagcagaaaaaga |
| | | ccagcagaaaaccagcagctagaaaatccacagcggcggcagcaggtggactgaggatcgc |
| | | ggcgaacgagccggcgcaaacccgggagctgaggaaccggatctttcccaccctctatgcca |
| | | tcttccagcagagtcgggggcaggagcaggaactgaaagtcaagaaccgttctctgcgctcgc |
| | | tcacccgcagttgtctgtatcacaagagcgaagaccaacttcagcgcactctcgaggacgccg |
| | | aggctctcttcaacaagtactgegcgctcactcttaaagagtagcccgcgcccgcccagtcgca |
| | | gaaaaaggcgggaattacgtcacctgtgcccttcgccctagccgcctccacccatcatcatgag |
| | | caaagagattcccacgccttacatgtggagctaccagccccagatgggcctggccgccggtgc |
| | | cgcccaggactactccacccgcatgaattggctcagcgccgggcccgcgatgatctcacgggt |
| | | gaatgacatccgcgcccaccgaaaccagatactcctagaacagtcagcgctcaccgccacgc |
| | | cccgcaatcacctcaatccgcgtaattggcccgccgccctggtgtaccaggaaattccccagcc |
| | | cacgaccgtactacttccgcgagacgcccaggccgaagtccagctgactaactcaggtgtcca |
| | | gctggcgggcggcgccaccctgtgtcgtcaccgccccgctcagggtataaagcggctggtga |
| | | tccggggcagaggcacacagctcaacgacgaggtggtgagctcttcgctgggtctgcgacct |
| | | gacggagtcttccaactcgccggatcggggagatcttccttcacgcctcgtcaggccgtcctga |
| | | ctttggagagttcgtcctcgcagccccgctcgggtggcatcggcactctccagttcgtggagga |
| | | gttcactccctcggtctacttcaaccccttctccggctcccccggccactacccggacgagttcat |
| | | cccgaacttcgacgccatcagcgagtcggtggacggctacgattgaaactaatcacccccttat |
| | | ccagtgaaataaagatcatattgatgatgattttacagaaataaaaaataatcatttgatttgaaata |
| | | aagatacaatcatattgatgatttgagtttaacaaaaaaataaagaatcacttacttgaaatctgata |
| | | ccaggtctctgtccatgttttctgccaacaccacttcactcccctcttcccagctctggtactgcag |
| | | gccccggcgggctgcaaacttcctccacacgctgaaggggatgtcaaattcctcctgtccctca |
| | | atcttcattttatcttctatcagatgtccaaaaagcgcgtccgggtggatgatgacttcgaccccgt |
| | | ctacccctacgatgcagacaacgcaccgaccgtgcccttcatcaacccccccttcgtctcttcag |
| | | atggattccaagagaagcccctgggggtgttgtccctgcgactggccgaccccgtcaccacca |
| | | agaacggggaaatcaccctcaagctgggagagggggtggacctcgattcctcgggaaaactc |
| | | atctccaacacggccaccaaggccgccgcccctctcagtttttccaacaacaccatttcccttaac |
| | | atggatcaccccttttacactaaagatggaaaattatccttacaagtttctccaccattaaatatactg |
| | | agaacaagcattctaaacacactagctttaggttttggatcaggtttaggactccgtggctctgcct |
| | | tggcagtacagttagtctctccacttacatttgatactgatggaaacataaagcttaccttagacag |
| | | aggtttgcatgttacaacaggagatgcaattgaaagcaacataagctgggctaaaggtttaaaatt |
| | | tgaagatggagccatagcaaccaacattggaaatgggttagagtttggaagcagtagtacagaa |
| | | acaggtgttgatgatgcttacccaatccaagttaaacttggatctggccttagctttgacagtacag |
| | | gagccataatggctggtaacaaagaagacgataaactcactttgtggacaacacctgatccatc |
| | | accaaactgtcaaatactcgcagaaaatgatgcaaaactaacactttgcttgactaaatgtggtag |
| | | tcaaatactggccactgtgtcagtcttagttgtaggaagtggaaacctaaaccccattactggcac |
| | | cgtaagcagtgctcaggtgtttctacgttttgatgcaaacggtgttcttttaacagaacattctacac |
| | | taaaaaaatactgggggtataggcagggagatagcatagatggcactccatataccaatgctgt |
| | | aggattcatgcccaatttaaaagcttatccaaagtcacaaagttctactactaaaaataatatagta |
| | | gggcaagtatacatgaatggagatgtttcaaaacctatgcttctcactataaccctcaatggtactg |
| | | atgacagcaacagtacatattcaatgtcattttcatacacctggactaatggaagctatgttggagc |
| | | aacatttggggctaactcttataccttctcatacatcgcccaagaatgaacactgtatcccaccctg |
| | | catgccaacccttcccaccccactctgtggaacaaactctgaaacacaaaataaaataaagttca |
| | | agtgttttattgattcaacagttttacaggattcgagcagttatttttcctccaccctcccaggacatg |
| | | gaatacaccaccctctccccccgcacagccttgaacatctgaatgccattggtgatggacatgct |
| | | tttggtctccacgttccacacagtttcagagcgagccagtctcgggtcggtcagggagatgaaa |
| | | ccctccgggcactcccgcatctgcacctcacagctcaacagctgaggattgtcctcggtggtcg |
| | | ggatcacggttatctggaagaagcagaagagcggcggtgggaatcatagtccgcgaacggga |
| | | tcggccggtggtgtcgcatcaggccccgcagcagtcgctgccgccgccgctccgtcaagctg |
| | | ctgctcagggggtccgggtccagggactccctcagcatgatgcccacggccctcagcatcagt |
| | | cgtctggtgcggcgggcgcagcagcgcatgcggatctcgctcaggtcgctgcagtacgtgca |
| | | acacagaaccaccaggttgttcaacagtccatagttcaacacgctccagccgaaactcatcgcg |
| | | ggaaggatgctacccacgtggccgtcgtaccagatcctcaggtaaatcaagtggtgccccctcc |
| | | agaacacgctgcccacgtacatgatctccttgggcatgtggcggttcaccacctcccggtacca |
| | | catcaccctctggttgaacatgcagccccggatgatcctgcggaaccacagggccagcaccgc |
| | | cccgcccgccatgcagcgaagagaccccgggtcccggcaatggcaatggaggacccaccg |
| | | ctcgtacccgtggatcatctgggagctgaacaagtctatgttggcacagcacaggcatatgctca |
| | | tgcatctcttcagcactctcaactcctcgggggtcaaaaccatatcccagggcacggggaactct |
| | | tgcaggacagcgaaccccgcagaacagggcaatcctcgcacagaacttacattgtgcatggac |
| | | agggtatcgcaatcaggcagcaccgggtgatcctccaccagagaagcgcgggtctcggtctc |
| | | ctcacagcgtggtaagggggccggccgatacgggtgatggcgggacgcggctgatcgtgttc |
| | | gcgaccgtgtcatgatgcagttgctttcggacattttcgtacttgctgtagcagaacctggtccgg |
| | | gcgctgcacaccgatcgccggcggcggtctcggcgcttggaacgctcggtgttgaaattgtaa |
| | | aacagccactctctcagaccgtgcagcagatctagggcctcaggagtgatgaagatcccatcat |
| | | gcctgatggctctgatcacatcgaccaccgtggaatgggccagacccagccagatgatgcaatt |
| | | ttgttgggtttcggtgacggcgggggagggaagaacaggaagaaccatgattaacttttaatcc |
| | | aaacggtctcggagtacttcaaaatgaagatcgcggagatggcacctctcgcccccgctgtgtt |
| | | ggtggaaaataacagccaggtcaaaggtgatacggttctcgagatgttccacggtggcttccag |
| | | caaagcctccacgcgcacatccagaaacaagacaatagcgaaagcgggagggttctctaattc |
| | | ctcaatcatcatgttacactcctgcaccatccccagataattttcatttttccagccttgaatgattcg |
| | | aactagttcctgaggtaaatccaagccagccatgataaagagctcgcgcagagcgccctccac |
| | | cggcattcttaagcacaccctcataattccaagatattctgctcctggttcacctgcagcagattga |
| | | caagcggaatatcaaaatctctgccgcgatccctgagctcctccctcagcaataactgtaagtac |
| | | tctttcatatcctctccgaaatttttagccataggaccaccaggaataagattagggcaagccaca |
| | | gtacagataaaccgaagtcctccccagtgagcattgccaaatgcaagactgctataagcatgct |
| | | ggctagacccggtgatatcttccagataactggacagaaaatcgcccaggcaatttttaagaaaa |
| | | tcaacaaaagaaaaatcctccaggtggacgtttagagcctcgggaacaacgatgaagtaaatg |
| | | caagcggtgcgttccagcatggttagttagctgatctgtagaaaaaacaaaaatgaacattaaac |
| | | catgctagcctggcgaacaggtgggtaaatcgttctctccagcaccaggcaggccacggggtc |
| | | tccggcgcgaccctcgtaaaaattgtcgctatgattgaaaaccatcacagagagacgttcccggt |
| | | ggccggcgtgaatgattcgacaagatgaatacacccccggaacattggcgtccgcgagtgaaa |
| | | aaaagcgcccgaggaagcaataaggcactacaatgctcagtctcaagtccagcaaagcgatg |
| | | ccatgcggatgaagcacaaaattctcaggtgcgtacaaaatgtaattactcccctcctgcacagg |
| | | cagcaaagcccccgatccctccaggtacacatacaaagcctcagcgtccatagcttaccgagc |
| | | agcagcacacaacaggcgcaagagtcagagaaaggctgagctctaacctgtccacccgctct |
| | | ctgctcaatatatagcccagatctacactgacgtaaaggccaaagtctaaaaatacccgccaaat |
| | | aatcacacacgcccagcacacgcccagaaaccggtgacacactcaaaaaaatacgcgcactt |
| | | cctcaaacgcccaaaactgccgtcatttccgggttcccacgctacgtcatcaaaacacgactttc |
| | | aaattccgtcgaccgttaaaaacgtcacccgccccgcccctaacggtcgcccgtctctcagcca |
| | | atcagcgccccgcatccccaaattcaaacacctcatttgcatattaacgcgcacaaaaagtttga |
| | | ggtatattattgatgatg |
| 536 | SIV Pol | atgcgcgtgtggccaatgggaaaagaagcaccacagttcccccatggcccagatgcaagtgg |
| | | agctgataccaactgcagccccagaggctctagctgcggcagcaccgaggaactgcacgag |
| | | gacgggcagaaggccgagggcgagcagcgggaaaccctccagggaggagatagaggcttt |
| | | gctgcccctcagttcagcctgtggcggagacccgtggtgaccgcctacatcgaggaacagccc |
| | | gtggaggtgctgctggccacaggcgctgacgacagcatcgtgaccggcatcgagctgggacc |
| | | caactacacccccaagatcgtgggaggaatcggaggattcatcaacaccggcgaatacaagg |
| | | acgtgaagatcaaggtgctcggcaaggtcatcaagggcaccatcatgacaggcgacaccccc |
| | | atcaacatctttggcagaaacctgctgacagccatgggcatgagcctgaacttccctatcgccaa |
| | | ggtggagcccatcaaggtgaccctgaagcctgggaaagaaggccctaagctgaggcagtgg |
| | | cctctgtccaaagagaagatcattgccctgcgggagatctgcgagaagatggaaaaggacgg |
| | | ccagctggaagaggctcctcccaccaacccctacaacacccccaccttcgccatcaagaagaa |
| | | agacaagaacaagtggcggatgctgatcgacttccgggagctgaataaggtgacccaggactt |
| | | caccgaggtgcagctgggcatccctcaccctgctggcctcgcaaagaggcggaggatcaccg |
| | | tcctggacgtgggcgacgcctactttagcattcccctggacgaagagttccggcagtacaccgc |
| | | cttcaccctgcccagcgtgaacaacgccgagcctggcaagcggtacatctacaaggtgctgcc |
| | | tcagggctggaagggctctcctgccatcttccagtacaccatgcggaacgtgctggaaccctttc |
| | | ggaaggccaaccctgacgtgaccctgatccagtacatggaccacatcctgatcgccagcgacc |
| | | ggaccgacctggaacatgacagagtcgtgctccagctgaaagagctgctgaacggcatcggc |
| | | ttcagcacacccgaagagaagttccagaaggaccctccctttcagtggatgggctatgaactct |
| | | ggcccaccaagtggaagctgcagaagatcgagctgccccagcgggagacatggaccgtgaa |
| | | cgacatccagaaactggtcggcgtgctgaactgggctgcccagatctaccctggcatcaagac |
| | | caagcacctgtgtcggctgatcagaggcaagatgaccctgaccgaggaagtgcagtggaccg |
| | | agatggccgaagccgagtacgaggaaaacaagatcatcctgagccaggaacaggaaggctg |
| | | ctactaccaggaaggcaagcccatcgaggccaccgtgatcaagagccaggacaaccagtgg |
| | | agctacaagatccaccaggaagataaagtcctgaaagtgggcaagttcgccaaagtgaagaac |
| | | acccacaccaacggcgtgcggctgctggcccacgtggtgcagaagattggcaaggaagccct |
| | | ggtgatctggggagaggtgcccaagttccacctgcccgtggagcgcgagatctgggagcagt |
| | | ggtggaccgactactggcaggtgacctggattcccgactgggatttcgtgagcacaccccctct |
| | | ggtccggctcgtgttcaacctggtgaaagagcctatccagggagccgagacattctacgtggac |
| | | ggcagctgcaaccggcagagcagggagggcaaggctggctatgtgaccgacaggggcagg |
| | | gacaaggccaagctgctggaacagaccaccaaccagcaggccgagctggaagccttctacct |
| | | ggccctggccgactctggccctaaggccaacatcatcgtggacagccagtacgtgatgggcat |
| | | cgtggctggacagcccaccgagagcgagtctcggctggtgaaccagatcatcgaggaaatga |
| | | tcaagaaagaggccatctacgtcgcctgggtgccagcccacaagggcattggaggaaaccag |
| | | gaagtggaccacctggtctcccagggcatccggcaggtgctgtttctggaaaaaatcgagcctg |
| | | cccaggaagagcacgagaagtaccacagcaacgtgaaagaactggtgtttaagttcggcatcc |
| | | ccagactggtggccaaacagattgtggacacctgcgaccggtgccaccagaagggcgaggc |
| | | catccacggccaggtcaacgccgagctgggcacctggcagatggcctgcacccacctggaa |
| | | ggcaagatcatcattgtggccgtgcacgtggccagcggcttcatcgaggccgaggtgatcccc |
| | | caggaaacaggacgccagacagccctgttcctgctgaagctggctggcaggtggcccatcac |
| | | ccacctgcacaccgacaatggcgctaacttcaccagccaggaagtgaagatggtcgcctggtg |
| | | ggctggcatcgaacagaccttcggcgtgccctacaacccccagagccagggcgtggtggag |
| | | gccatgaaccaccacctgaaaacccagatcgaccggatcagagagcaggccaacagcgtcg |
| | | agacaatcgtgctgatggccgtgcactgcatgaacttcaagcggagaggagggatcggcgac |
| | | atgacccctgccgagagactggtgaacatgatcaccaccgagcaggaaatccagttccagcag |
| | | agcaagaacagcaagttcaagaacttccgggtgtactaccgggagggcagggaccagctgtg |
| | | gagaggccctggcgagctgctgtggaagggcgaaggcgctgtgatcctgaaagtcgggacc |
| | | gagatcaaggtggtgcccaggcggaaggccaagattatcaaggactatggcggagggaaag |
| | | agctggacagcggctctcacctggaagatacaggcgaggcacgggaggtggcctaa |
| 537 | SAM-SIVEnv | atgggcggcgcatgagagaagcccagaccaattacctacccaaaatggagaaagttcacgttg |
| | (uppercase, | acatcgaggaagacagcccattcctcagagctttgcagcggagcttcccgcagtttgaggtaga |
| | bold, | agccaagcaggtcactgataatgaccatgctaatgccagagcgttttcgcatctggcttcaaaac |
| | underline | tgatcgaaacggaggtggacccatccgacacgatccttgacattggaagtgcgcccgcccgca |
| | ENV = nt 7571 | gaatgtattctaagcacaagtatcattgtatctgtccgatgagatgtgcggaagatccggacagat |
| | to 9673; | tgtataagtatgcaactaagctgaagaaaaactgtaaggaaataactgataaggaattggacaag |
| | uppercase, | aaaatgaaggagctcgccgccgtcatgagcgaccctgacctggaaactgagactatgtgcctc |
| | bold, italic, no | cacgacgacgagtcgtgtcgctacgaagggcaagtcgctgtttaccaggatgtatacgcggttg |
| | underline = | acggaccgacaagtctctatcaccaagccaataagggagttagagtcgcctactggataggctt |
| | 26S | tgacaccaccccttttatgtttaagaacttggctggagcatatccatcatactctaccaactgggcc |
| | subgenomic | gacgaaaccgtgttaacggctcgtaacataggcctatgcagctctgacgttatggagcggtcac |
| | promoter = nt | gtagagggatgtccattcttagaaagaagtatttgaaaccatccaacaatgttctattctctgttggc |
| | 7513 to 7536; | tcgaccatctaccacgagaagagggacttactgaggagctggcacctgccgtctgtatttcactt |
| | uppercase, | acgtggcaagcaaaattacacatgtcggtgtgagactatagttagttgcgacgggtacgtcgtta |
| | bold, dashed | aaagaatagctatcagtccaggcctgtatgggaagccttcaggctatgctgctacgatgcaccg |
| | underline = | cgagggattcttgtgctgcaaagtgacagacacattgaacggggagagggtctcttttcccgtgt |
| | polyA = nt | gcacgtatgtgccagctacattgtgtgaccaaatgactggcatactggcaacagatgtcagtgcg |
| | 9960 to | gacgacgcgcaaaaactgctggttgggctcaaccagcgtatagtcgtcaacggtcgcacccag |
| | 10039) | agaaacaccaataccatgaaaaattaccttttgcccgtagtggcccaggcatttgctaggtgggc |
| | | aaaggaatataaggaagatcaagaagatgaaaggccactaggactacgagatagacagttagt |
| | | catggggtgttgttgggcttttagaaggcacaagataacatctatttataagcgcccggataccca |
| | | aaccatcatcaaagtgaacagcgatttccactcattcgtgctgcccaggataggcagtaacacat |
| | | tggagatcgggctgagaacaagaatcaggaaaatgttagaggagcacaaggagccgtcacct |
| | | ctcattaccgccgaggacgtacaagaagctaagtgcgcagccgatgaggctaaggaggtgcg |
| | | tgaagccgaggagttgcgcgcagctctaccacctttggcagctgatgttgaggagcccactctg |
| | | gaagccgatgtcgacttgatgttacaagaggctggggccggctcagtggagacacctcgtggc |
| | | ttgataaaggttaccagctacgctggcgaggacaagatcggctcttacgctgtgctttctccgca |
| | | ggctgtactcaagagtgaaaaattatcttgcatccaccctctcgctgaacaagtcatagtgataac |
| | | acactctggccgaaaagggcgttatgccgtggaaccataccatggtaaagtagtggtgccaga |
| | | gggacatgcaatacccgtccaggactttcaagctctgagtgaaagtgccaccattgtgtacaac |
| | | gaacgtgagttcgtaaacaggtacctgcaccatattgccacacatggaggagcgctgaacactg |
| | | atgaagaatattacaaaactgtcaagcccagcgagcacgacggcgaatacctgtacgacatcg |
| | | acaggaaacagtgcgtcaagaaagaactagtcactgggctagggctcacaggcgagctggtg |
| | | gatcctcccttccatgaattcgcctacgagagtctgagaacacgaccagccgctccttaccaagt |
| | | accaaccataggggtgtatggcgtgccaggatcaggcaagtctggcatcattaaaagcgcagt |
| | | caccaaaaaagatctagtggtgagcgccaagaaagaaaactgtgcagaaattataagggacgt |
| | | caagaaaatgaaagggctggacgtcaatgccagaactgtggactcagtgctcttgaatggatgc |
| | | aaacaccccgtagagaccctgtatattgacgaagcttttgcttgtcatgcaggtactctcagagcg |
| | | ctcatagccattataagacctaaaaaggcagtgctctgcggggatcccaaacagtgcggttttttt |
| | | aacatgatgtgcctgaaagtgcattttaaccacgagatttgcacacaagtcttccacaaaagcatc |
| | | tctcgccgttgcactaaatctgtgacttcggtcgtctcaaccttgttttacgacaaaaaaatgagaa |
| | | cgacgaatccgaaagagactaagattgtgattgacactaccggcagtaccaaacctaagcagg |
| | | acgatctcattctcacttgtttcagagggtgggtgaagcagttgcaaatagattacaaaggcaac |
| | | gaaataatgacggcagctgcctctcaagggctgacccgtaaaggtgtgtatgccgttcggtaca |
| | | aggtgaatgaaaatcctctgtacgcacccacctcagaacatgtgaacgtcctactgacccgcac |
| | | ggaggaccgcatcgtgtggaaaacactagccggcgacccatggataaaaacactgactgcca |
| | | agtaccctgggaatttcactgccacgatagaggagtggcaagcagagcatgatgccatcatga |
| | | ggcacatcttggagagaccggaccctaccgacgtcttccagaataaggcaaacgtgtgttggg |
| | | ccaaggctttagtgccggtgctgaagaccgctggcatagacatgaccactgaacaatggaaca |
| | | ctgtggattattttgaaacggacaaagctcactcagcagagatagtattgaaccaactatgcgtga |
| | | ggttctttggactcgatctggactccggtctattttctgcacccactgttccgttatccattaggaata |
| | | atcactgggataactccccgtcgcctaacatgtacgggctgaataaagaagtggtccgtcagct |
| | | ctctcgcaggtacccacaactgcctcgggcagttgccactggaagagtctatgacatgaacact |
| | | ggtacactgcgcaattatgatccgcgcataaacctagtacctgtaaacagaagactgcctcatgc |
| | | tttagtcctccaccataatgaacacccacagagtgacttttcttcattcgtcagcaaattgaagggc |
| | | agaactgtcctggtggtcggggaaaagttgtccgtcccaggcaaaatggttgactggttgtcag |
| | | accggcctgaggctaccttcagagctcggctggatttaggcatcccaggtgatgtgcccaaatat |
| | | gacataatatttgttaatgtgaggaccccatataaataccatcactatcagcagtgtgaagaccat |
| | | gccattaagcttagcatgttgaccaagaaagcttgtctgcatctgaatcccggcggaacctgtgtc |
| | | agcataggttatggttacgctgacagggccagcgaaagcatcattggtgctatagcgcggcagt |
| | | tcaagttttcccgggtatgcaaaccgaaatcctcacttgaagagacggaagttctgtttgtattcatt |
| | | gggtacgatcgcaaggcccgtacgcacaatccttacaagctttcatcaaccttgaccaacatttat |
| | | acaggttccagactccacgaagccggatgtgcaccctcatatcatgtggtgcgaggggatattg |
| | | ccacggccaccgaaggagtgattataaatgctgctaacagcaaaggacaacctggcggaggg |
| | | gtgtgcggagcgctgtataagaaattcccggaaagcttcgatttacagccgatcgaagtaggaa |
| | | aagcgcgactggtcaaaggtgcagctaaacatatcattcatgccgtaggaccaaacttcaacaa |
| | | agtttcggaggttgaaggtgacaaacagttggcagaggcttatgagtccatcgctaagattgtca |
| | | acgataacaattacaagtcagtagcgattccactgttgtccaccggcatcttttccgggaacaaag |
| | | atcgactaacccaatcattgaaccatttgctgacagctttagacaccactgatgcagatgtagcca |
| | | tatactgcagggacaagaaatgggaaatgactctcaaggaagcagtggctaggagagaagca |
| | | gtggaggagatatgcatatccgacgactcttcagtgacagaacctgatgcagagctggtgagg |
| | | gtgcatccgaagagttctttggctggaaggaagggctacagcacaagcgatggcaaaactttct |
| | | catatttggaagggaccaagtttcaccaggcggccaaggatatagcagaaattaatgccatgtg |
| | | gcccgttgcaacggaggccaatgagcaggtatgcatgtatatcctcggagaaagcatgagcag |
| | | tattaggtcgaaatgccccgtcgaagagtcggaagcctccacaccacctagcacgctgccttgc |
| | | ttgtgcatccatgccatgactccagaaagagtacagcgcctaaaagcctcacgtccagaacaaa |
| | | ttactgtgtgctcatcctttccattgccgaagtatagaatcactggtgtgcagaagatccaatgctc |
| | | ccagcctatattgttctcaccgaaagtgcctgcgtatattcatccaaggaagtatctcgtggaaac |
| | | accaccggtagacgagactccggagccatcggcagagaaccaatccacagaggggacacct |
| | | gaacaaccaccacttataaccgaggatgagaccaggactagaacgcctgagccgatcatcatc |
| | | gaagaggaagaagaggatagcataagtttgctgtcagatggcccgacccaccaggtgctgca |
| | | agtcgaggcagacattcacgggccgccctctgtatctagctcatcctggtccattcctcatgcatc |
| | | cgactttgatgtggacagtttatccatacttgacaccctggagggagctagcgtgaccagcggg |
| | | gcaacgtcagccgagactaactcttacttcgcaaagagtatggagtttctggcgcgaccggtgc |
| | | ctgcgcctcgaacagtattcaggaaccctccacatcccgctccgcgcacaagaacaccgtcact |
| | | tgcacccagcagggcctgctcgagaaccagcctagtttccaccccgccaggcgtgaatagggt |
| | | gatcactagagaggagctcgaggcgcttaccccgtcacgcactcctagcaggtcggtctcgag |
| | | aaccagcctggtctccaacccgccaggcgtaaatagggtgattacaagagaggagtttgaggc |
| | | gttcgtagcacaacaacaatgacggtttgatgcgggtgcatacatcttttcctccgacaccggtca |
| | | agggcatttacaacaaaaatcagtaaggcaaacggtgctatccgaagtggtgttggagaggac |
| | | cgaattggagatttcgtatgccccgcgcctcgaccaagaaaaagaagaattactacgcaagaaa |
| | | ttacagttaaatcccacacctgctaacagaagcagataccagtccaggaaggtggagaacatga |
| | | aagccataacagctagacgtattctgcaaggcctagggcattatttgaaggcagaaggaaaagt |
| | | ggagtgctaccgaaccctgcatcctgttcctttgtattcatctagtgtgaaccgtgccttttcaagcc |
| | | ccaaggtcgcagtggaagcctgtaacgccatgttgaaagagaactttccgactgtggcttcttac |
| | | tgtattattccagagtacgatgcctatttggacatggttgacggagcttcatgctgcttagacactg |
| | | ccagtttttgccctgcaaagctgcgcagctttccaaagaaacactcctatttggaacccacaatac |
| | | gatcggcagtgccttcagcgatccagaacacgctccagaacgtcctggcagctgccacaaaaa |
| | | gaaattgcaatgtcacgcaaatgagagaattgcccgtattggattcggcggcctttaatgtggaat |
| | | gcttcaagaaatatgcgtgtaataatgaatattgggaaacgtttaaagaaaaccccatcaggctta |
| | | ctgaagaaaacgtggtaaattacattaccaaattaaaaggaccaaaagctgctgctctttttgcga |
| | | agacacataatttgaatatgttgcaggacataccaatggacaggtttgtaatggacttaaagagag |
| | | acgtgaaagtgactccaggaacaaaacatactgaagaacggcccaaggtacaggtgatccag |
| | | gctgccgatccgctagcaacagcgtatctgtgcggaatccaccgagagctggttaggagattaa |
| | | atgcggtcctgcttccgaacattcatacactgtttgatatgtcggctgaagactttgacgctattata |
| | | gccgagcacttccagcctggggattgtgttctggaaactgacatcgcgtcgtttgataaaagtga |
| | | ggacgacgccatggctctgaccgcgttaatgattctggaagacttaggtgtggacgcagagctg |
| | | ttgacgctgattgaggcggctttcggcgaaatttcatcaatacatttgcccactaaaactaaatttaa |
| | | attcggagccatgatgaaatctggaatgttcctcacactgtttgtgaacacagtcattaacattgta |
| | | atcgcaagcagagtgttgagagaacggctaaccggatcaccatgtgcagcattcattggagatg |
| | | acaatatcgtgaaaggagtcaaatcggacaaattaatggcagacaggtgcgccacctggttgaa |
| | | tatggaagtcaagattatagatgctgtggtgggcgagaaagcgccttatttctgtggagggtttatt |
| | | ttgtgtgactccgtgaccggcacagcgtgccgtgtggcagaccccctaaaaaggctgtttaagc |
| | | ttggcaaacctctggcagcagacgatgaacatgatgatgacaggagaagggcattgcatgaag |
| | | agtcaacacgctggaaccgagtgggtattctttcagagctgtgcaaggcagtagaatcaaggta |
| | | tgaaaccgtaggaacttccatcatagttatggccatgactactctagctagcagtgttaaatcattc |
| | | agctacctgagaggggcccctataaCTCTCTACGGCTAACCTGAATGGAc |
| | | tacgactctagaatagtctttaatTAAGCCACCATGGGCTGCCTGGGCA |
| | | ACCAGCTGCTGATCGCCCTGCTGCTGGTGTCCGTGCT |
| | | GGAAATCTGCTGCGTGCAGTACGTGACCGTGTTCTAC |
| | | GGCGTGCCTGCCTGGAAGAACGCCACCATCCCCCTGT |
| | | TCTGCGCCACCCGGAACCGGGACACCTGGGGCACCAC |
| | | ACAGTGCCTGCCCGACAACGACGACTACAGCGAGCTG |
| | | GCCGTGAACATCACCGAGGCCTTCGACGCCTGGAACA |
| | | ACACCGTGACCGAGCAGGCCATCGAGGACGTGTGGAA |
| | | CCTGTTCGAGACAAGCATCAAGCCCTGCGTGAAGCTG |
| | | ACACCCCTGTGTATCGCCATGCGGTGCAACAAGACCG |
| | | AGACAGACAGATGGGGACTGACCGGCAGGGCTGAGA |
| | | CAACCACCACCGCCAAGAGCACCACCTCTACCACCAC |
| | | CACAACCGTGACCCCCAAGGTGATCAACGAGGGCGAC |
| | | AGCTGCATCAAGAACAACAGCTGTGCTGGCCTCGAAC |
| | | AGGAACCCATGATCGGCTGCAAGTTCAACATGACAGG |
| | | CCTGAAGCGGGACAAGAAGATCGAGTACAACGAGACA |
| | | TGGTACTCTCGGGACCTGATCTGCGAGCAGCCTGCCA |
| | | ACGGCAGCGAGAGCAAGTGTTACATGCAGCATTGCAA |
| | | CACCAGCGTGATCCAGGAAAGCTGCGACAAGCACTAC |
| | | TGGGACGCCATCCGGTTCCGGTACTGCGCTCCTCCTG |
| | | GCTACGCCCTGCTGAGGTGCAACGACAGCAACTACAG |
| | | CGGCTTCGCTCCCAAGTGCAGCAAGGTGGTGGTGTCC |
| | | AGCTGCACCCGGATGATGGAAACCCAGACCAGCACCT |
| | | GGTTCGGCTTCAACGGCACCAGAGCCGAGAACCGGAC |
| | | CTACATCTACTGGCACGGCAACAGCAACCGGACCATC |
| | | ATCAGCCTGAACAAGTACTACAACCTGACCATGAAGT |
| | | GCAGAAGGCCTGGCAATAAGACCGTGCTGCCCGTGAC |
| | | CATCATGTCTGGCCTGGTGTTCCACAGCCAGCCCATC |
| | | AACGAGAGGCCCAAGCAGGCCTGGTGCCGGTTCGGAG |
| | | GGAACTGGTCCGAGGCCATCCAGGAAGTGAAAGAAAC |
| | | CCTGGTGAAGCACCCTCGGTACACCGGCACCAACGAC |
| | | ACCCGGAAGATCAACCTGACCGCTCCTGCTGGCGGAG |
| | | ATCCCGAGGTGACCTTCATGTGGACCAACTGCAGAGG |
| | | CGAGTTCCTGTACTGCAAGATGAACTGGTTCCTGAACT |
| | | GGGTGGAGGACCGGGACCAGAACAGCAACAGGTGGA |
| | | AGCAGCAGAAGAAGCCCGAGCAGCAGAAACGGAACTA |
| | | CGTGCCCTGCCACATCCGGCAGATCATCAACACCTGG |
| | | CACAAAGTGGGCAAGAACGTGTACCTGCCTCCCAGAG |
| | | AGGGCGACCTGACCTGCAACTCCACCGTGACCAGCCT |
| | | GATCGCCGAGATCGACTGGATCAACAACAACGAAACC |
| | | AACATCACCATGTCTGCCGAGGTGGCCGAGCTGTACA |
| | | GGCTGGAACTGGGCGACTACAAACTGGTGGAGATCAC |
| | | CCCCATCGGCCTGGCTCCCACCGACGTCAGACGGTAC |
| | | ACCACCACAGGAGCCAGCCGGAACAAGAGAGGCGTCT |
| | | TTGTCCTGGGCTTCCTCGGCTTTCTGGCCACAGCAGG |
| | | CTCTGCAATGGGAGCAGCCAGCCTGACCCTGTCTGCC |
| | | CAGAGCCGGACACTGCTGGCAGGCATTGTGCAGCAGC |
| | | AGCAACAGCTCCTGGACGTGGTGAAGCGGCAGCATGA |
| | | ACTCCTGCGACTGACCGTGTGGGGCACCAAGAACCTG |
| | | CAGACACGGGTGACCGCCATCGAGAAGTACCTGAAGG |
| | | ACCAGGCTCAGCTCAACTCTTGGGGCTGCGCCTTCCG |
| | | GCAGGTCTGCCACACCACCGTGCCCTGGCCCAACGAC |
| | | TCCCTGGTGCCCAACTGGGACAACATGACCTGGCAGG |
| | | AATGGGAGGGCAAGGTCGACTTCCTGGAAGCCAATAT |
| | | CACCCAGCTGCTGGAAGAGGCCCAGATCCAGCAGGAA |
| | | AAGAACATGTACGAGCTGCAGAAGCTGAACTCCTGGG |
| | | ACATCTTCGGCAATTGGTTCGACCTGACCTCCTGGATT |
| | | AGATACATCCAGTAAtgattcgaacggccgtatcacgcccaaacatttacagcc |
| | | gcggtgtcaaaaaccgcgtggacgtggttaacatccctgctgggaggatcagccgtaattattat |
| | | aattggcttggtgctggctactattgtggccatgtacgtgctgaccaaccagaaacataattgaat |
| | | acagcagcaattggcaagctgcttacatagaactcgcggcgattggcatgccgccttaaaattttt |
| | | attttattttttcttttcttttccgaatcggattttgtttttaatatttc |
| | | aaaaaaaaaaaaaaaaaaaaaaaaaaaaaaaaaaaaaaaaaaaaaaaaaaaaaa |
| | | aaaaaaaaaaaaaaaaaaaaaaaaaa |
| 538 | SIV Gag | atgggagccaggaacagtgtgctctcaggcaagaaggcagatgagctggagaagatcaggct |
| | | gagacccaatggcaagaagaagtacatgctgaagcatgtggtctgggcagccaatgagctgg |
| | | acaggtttggcctggcagagtccctgctggacaacaaggagggctgccagaagatcctgtcag |
| | | tgctggcccccctggtgcccactggctcagagaacctgaagagtctctacaacacagtgtgtgt |
| | | gatttggtgcatccatgcagaggagaaggtgaagcacactgaggaggccaagcagattgtgca |
| | | gaggcacctggtggtggagactggcacagctgacaagatgccagccacctccaggcccaca |
| | | gcacccccctctggcagggggggcaactacccagtccagcaagtggggggcaactatgtgca |
| | | cctgcccctgagccccagaaccctgaatgcctgggtcaagctggtggaggagaagaagtttgg |
| | | agcagaggtggtgcctggcttccaggccctgtcagagggatgcactccctatgacatcaacca |
| | | gatgctgaactgtgtgggagagcaccaggcagcaatgcagatcatcagggagatcatcaatga |
| | | ggaggctgctgactgggacctccagcacccccagcctggacccctccctgcaggccagctga |
| | | gggagcccagagggagtgacatagcaggcaccacctccacagtggaggagcagatccagtg |
| | | gatgtacaggcagcagaaccccatccctgtgggcaacatctacaggaggtggatccagctgg |
| | | gcctccagaagtgtgtcaggatgtacaacccaaccaacatcctggatgtgaagcagggcccca |
| | | aggagcccttccagtcttatgtggacaggttctacaagagcctgagagctgagcagacagacc |
| | | ctgctgtgaagaactggatgacccagacactgctgatccagaatgccaatcctgactgcaagct |
| | | ggtgctgaaggggctggggatgaatccaaccctggaggagatgctgacagcctgccagggc |
| | | attgggggacctggacagaaggccaggctcatggcagaggctctcaaggaggccctcagac |
| | | cagaccagctgccatttgctgctgtgcagcagaagggccagaggaggaccatcaagtgctgg |
| | | aactgtggcaaggagggccactctgccaggcagtgcagagcccccaggaggcagggctgct |
| | | ggggctgtggaaagacaggccatgtgatggccaagtgcccagagaggcaggcaggcttcct |
| | | gggctttggcccctggggcaagaagccaagaaacttccccatggcccagatgccccagggcc |
| | | tgacccccacagcccccccagaggacccagctgtggacctgctgaagaactacatgaagatg |
| | | ggcaggaagcagagggagaacagggagagaccctacaaggaggtgactgaggacctgctg |
| | | cacctgaactccctgtttggggaggaccagtga |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A self-amplifying RNA (SAM or saRNA) comprising:
a. a polynucleotide comprising any one of the nucleic acid sequences of SEQ ID NOS: 522-523;
b. a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 522-523, wherein percent identity is across the full length of the nucleic acid sequence of SEQ ID NOs: 522-523;
c. a polynucleotide comprising any one of the nucleic acid sequences of SEQ ID NOs: 524-526;
d. a polynucleotide comprising a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526;
e. a polynucleotide that encodes any one of the amino acid sequences of SEQ ID NOs: 527-528;
f. a polynucleotide that encodes a polypeptide that is at least 80%, 85%, 86%, 87%, 88%, 89%, 90%, 91%, 92%, 93%, 94%, 95%, 96%, 97%, 98%, 99%, or 100% identical to any one of the amino acid sequences of SEQ ID NOs: 527-528, wherein percent identity is across the full length of the amino acid sequence of SEQ ID NOs: 527-528;
g. (i) a first polynucleotide sequence comprising nucleotides 1 to 7512 of SEQ ID NO: 522; (ii) a promoter sequence; (iii) a second polynucleotide sequence comprising nucleotides 7537 to 7570 of SEQ ID NO: 522; (iv) a third polynucleotide sequence that encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (v) a fourth polynucleotide sequence comprising nucleotides 10667 to 10952 of SEQ ID NO: 522; and (vi) a polyA region; or
h. (i) a first polynucleotide sequence comprising nucleotides 1 to 7512 of SEQ ID NO: 522; (ii) a promoter sequence; (iii) a second polynucleotide sequence comprising nucleotides 7537 to 7570 of SEQ ID NO: 522; (iv) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (v) a fourth polynucleotide sequence comprising nucleotides 10667 to 10952 of SEQ ID NO: 522; and (vi) a polyA region.

2. The SAM of claim 1 (h)(iv), wherein the third polynucleotide sequence encodes two or more polypeptide segments.

3. The SAM of claim 1 (h)(iv), wherein the third polynucleotide sequence encodes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344.

4. The SAM of claim 2 or 3, wherein:
a. one or more of the polypeptide segments is abutted or fused to an adjacent polypeptide segment;
b. one or more of the polypeptide segments is joined to an adjacent polypeptide segment by one or more peptide linkers.

5. The SAM of claim 4, wherein the one or more peptide linkers is selected from one or more of a polyalanine linker, a polyglycine linker, a cleavable linker, a flexible linker, a rigid linker, a Nef linking sequence, and combinations thereof.

6. The SAM of claim 5, wherein:
a. the polyalanine linker comprises or consists of 2 or 3 contiguous alanine residues, e.g. AA, AAA, AAY or AAX, wherein X is any amino acid (e.g. A, C, D, E, F, G, H, I, K, L, M, N, P, Q, R, S, T, V, Y);
b. the flexible linker or polyglycine linker comprises or consists of GG, GGS, GSG or GGGS (SEQ ID NO: 421),
c. the cleavable linker is selected from a 2A cleavable peptide (e.g., foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof;
d. the cleavable linker comprises or consists of a furin recognition/cleavage site selected from the group consisting of RAKR (SEQ ID NO: 381), REKR (SEQ ID NO: 382) and RRKR (SEQ ID NO: 383);
e. the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388);
f. the cleavable linker comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388);
g. the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), wherein percent identity is across the full length of SEQ ID NOs: 389-391; and/or
h. the Nef linking sequence comprises or consists of an amino acid sequence selected from VHAGPIA (SEQ ID NO: 389), VHAGPVA (SEQ ID NO: 390) and GALDI (SEQ ID NO: 391).

7. The SAM of any one of claims 1 to 6, wherein the promoter sequence comprises a polynucleotide sequence of SEQ ID NO: 529.

8. An expression cassette, comprising the SAM of any one of claims 1 to 7, operably linked to one or more regulatory sequences.

9. The expression cassette of claim 8, wherein the polynucleotide is operably linked to and under the control of a constitutive promoter.

10. The expression cassette of claim 8, wherein the promoter is selected from a CMV promoter, a CAG promoter, an EF1a promoter, and a 26S sub-genomic promoter.

11. The expression cassette of any one of claims 8 to 10, wherein the promoter comprises any of the promoter sequences of SEQ ID NOs: 529-530.

12. A lipid nanoparticle (LNP) comprising the SAM of any one of claims 1 to 7, or the expression cassette of any one of claims 8 to 11.

13. The LNP of claim 12, wherein the LNP comprises or is synthesized from an ionizable lipid, phospholipid, cholesterol, PEGylated lipid, or any combination thereof.

14. The LNP of claim 12 or 13, wherein the LNP comprises or is synthesized from cationic lipid 1,2-dioleoyl-3-timethylammonium-propane (DOTAP).

15. A polymeric nanoparticle (PNP) comprising the SAM of any one of claims 1 to 7, or the expression cassette of any one of claims 8 to 11.

16. The PNP of claim 15, wherein the PNP comprises or is synthesized from non-degradable polymers, degradable polymers, natural materials, synthetic materials, monomers, dendrimers, or any combination thereof.

17. The PNP of claim 15 or 16, wherein the PNP comprises or is synthesized from poly(ethylene glycol) (PEG), poly(dimethylsiloxane) (PDMS), polyethyleneimine (PEI), poly(amidoamine) (PAMAM), poly(dimethylaminoethyl) acrylate (pDMAEA), orthenine-derived dendrimers, or any combination thereof.

18. The PNP of any one of claims 15 to 17, wherein the PNP comprises or is synthesized from mannosylated-PEI polyplexes.

19. The PNP of any one of claims 15 to 18, wherein the PNP comprises or is synthesized from a bioreducible, linear, cationic polymer (pABOL).

20. A nanoemulsion comprising the SAM of any one of claims 1 to 7, or the expression cassette of any one of claims 8 to 11.

21. The nanoemulsion of claim 20, wherein the nanoemulsion is a water-in-oil emulsion.

22. The nanoemulsion of claim 20, wherein the nanoemulsion comprises squalene, sorbitan trioleate, polysorbate 80, DOTAP, or any combination thereof.

23. An expression vector comprising the SAM of any one of claims 1 to 7, or the expression cassette of any one of claims 8 to 10.

24. The expression vector of claim 23, wherein the expression vector is a plasmid vector, a bacterial vector or a viral expression vector.

25. The expression vector of claim 23 or 24, wherein the expression vector is a viral expression vector.

26. The expression vector of claim 25, wherein the viral expression vector is a DNA virus or an RNA virus.

27. The expression vector of any one of claims 25 to 26, wherein the viral expression vector is replication defective, replication deficient, replication attenuated or replication competent.

28. The expression vector of any one of claims 25 to 27, wherein the viral expression vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus.

29. The expression vector of any one of claims 25 to 28, wherein the viral expression vector is from a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus).

30. The expression vector of any one of claims 25 to 29, wherein the viral expression vector is an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV).

31. The expression vector of claim 30, wherein the viral expression vector is an arenavirus vector selected from Lymphocytic choriomeningitis mammarenavirus (LCMV) or Cali mammarenavirus (a.k.a. Pichinde mammarenavirus or Pichinde arenavirus).

32. The expression vector of claim 30 or 31, wherein the arenavirus vector comprises a bi-segmented genome.

33. The expression vector of claim 30 or 31, wherein the arenavirus vector comprises a tri-segmented genome.

34. The expression vector of any one of claims 25 to 29, wherein the viral expression vector is a human adenovirus or a simian adenovirus.

35. The expression vector of claim 34, wherein the simian adenovirus is selected from a chimpanzee adenovirus, a gorilla adenovirus, and a rhesus adenovirus.

36. The expression vector of any one of claims 25 to 29 and 34, wherein the viral expression vector is an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (ChAd), gorilla adenovirus, and rhesus adenovirus.

37. The expression vector of claim 36, wherein the ChAd is selected from ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), and ChAd147 (AdC147).

38. The expression vector of claim 37, wherein the ChAd is ChAd68.

39. The expression vector of claim 36, wherein the gorilla adenovirus is selected from GC44, GC45, and GC46.

40. The expression vector of claim 36, wherein the rhesus adenovirus (RhAd) is selected from RhAd51, RhAd52, RhAd53, RhAd54, RhAd55, RhAd56, RhAd57, RhAd58, RhAd59, RhAd60, RhAd61, RhAd62, RhAd63, RhAd64, RhAd65 and RhAd66.

41. The expression vector of any one of claims 34 to 40, wherein the human adenovirus or simian adenovirus comprises a modification of one or more adenoviral genes.

42. The expression vector of claim 41, wherein the one or more adenoviral genes are selected from E1, E3, and E4ORF2-4.

43. The expression vector of claim 41 or 42, wherein the modification comprises a deletion, substitution, or insertion of one or more nucleotides into the one or more adenoviral genes.

44. The expression vector of any one of claims 41 to 43, wherein the human adenovirus or simian adenovirus comprises a deletion of at least a portion of the E1 region.

45. The expression vector of claim 44, wherein the polynucleotide is inserted into the deletion of at least a portion of the E1 region.

46. The expression vector of any one of claims 23 to 45, wherein the expression vector comprises any one of the nucleic acid sequences of SEQ ID NOs: 520-521.

47. The expression vector of any one of claims 23 to 45, wherein the expression vector comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521.

48. An expression vector comprising:
a. any one of the nucleic acid sequences of SEQ ID NOs: 524-526;
b. a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 524-526, wherein percent identity is across the full length of the nucleic acid sequence of SEQ ID NOs: 524-526;
c. a polynucleotide sequence that encodes for any one of the amino acid sequences of SEQ ID NOs: 527-528;
d. a polynucleotide sequence that encodes for polynucleotide that is at least 80%, 85%, 86%, 87%, 88%, 89%, 90%, 91%, 92%, 93%, 94%, 95%, 96%, 97%, 98%, 99%, or 100% identical to any one of the amino acid sequences of SEQ ID NOs: 527-528, wherein percent identity is across the full length of the amino acid sequence of SEQ ID NOs: 527-528,
e. (i) a first polynucleotide sequence comprising nucleotides 1 to 593 of SEQ ID NO: 520; (ii) a promoter sequence; (iii) a second polynucleotide sequence comprising nucleotides 1196 to 1255 of SEQ ID NO: 520; (iv) a third polynucleotide sequence encodes a polypeptide comprising an amino acid sequence that is at least 90%, 91%, 92%, 93%, 94%, 95%, 96%, 97%, 98% or at least 99% identical to the amino acid sequence of any one of SEQ ID NOs: 345-371, 373-377, 407-411, 422-423, 430-435, 527, and 528, wherein percent identity is across the full length of SEQ ID NOs: 345-371, 373-377, 407-411, 422-423, 430-435, 527, and 528; (v) a fourth polynucleotide sequence comprising nucleotides 4352 to 4395 of SEQ ID NO: 520; (vi) a polyA sequence; and (vii) a fifth polynucleotide sequence comprising nucleotides 4620 to 32350 of SEQ ID NO: 520; or
f. (i) a first polynucleotide sequence comprising nucleotides 1 to 593 of SEQ ID NO: 520; (ii) a promoter sequence; (iii) a second polynucleotide sequence comprising nucleotides 1196 to 1255 of SEQ ID NO: 520; (iv) a third polynucleotide sequence that encodes one or more polypeptide segments, wherein the one or more polypeptide segments comprise an amino acid sequence that is at least 90%, 91%, 92%, 93%, 94%, 95%, 96%, 97%, 98% or at least 99% identical to the amino acid sequence of any one of SEQ ID NOs: 1-344, wherein percent identity is across the full length of SEQ ID NOs: 1-344; (v) a fourth polynucleotide sequence comprising nucleotides 4352 to 4395 of SEQ ID NO: 520; (vi) a polyA sequence; and (vii) a fifth polynucleotide sequence comprising nucleotides 4620 to 32350 of SEQ ID NO: 520.

49. The expression vector of claim 48, wherein the third polynucleotide sequence encodes two or more polypeptide segments.

50. The expression vector of claim 48, wherein the third polynucleotide sequence encodes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344.

51. The expression vector of claim 49 or 50, wherein one or more of the polypeptide segments is abutted or fused to an adjacent polypeptide segment.

52. The expression vector of claim 49 or 50, wherein one or more of the polypeptide segments is joined to an adjacent polypeptide segment by one or more peptide linkers.

53. The expression vector of claim 52, wherein the one or more peptide linkers is selected from one or more of a polyalanine linker, a polyglycine linker, a cleavable linker, a flexible linker, a rigid linker, a Nef linking sequence, and combinations thereof.

54. The expression vector of claim 53, wherein:
a. the polyalanine linker comprises or consists of 2 or 3 contiguous alanine residues, e.g. AA, AAA, AAY or AAX, wherein X is any amino acid (e.g. A, C, D, E, F, G, H, I, K, L, M, N, P, Q, R, S, T, V, Y);
b. the flexible linker or polyglycine linker comprises or consists of GG, GGS, GSG or GGGS (SEQ ID NO: 421);
c. the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof;
d. the cleavable linker comprises or consists of a furin recognition/cleavage site selected from the group consisting of RAKR (SEQ ID NO: 381), REKR (SEQ ID NO: 382) and RRKR (SEQ ID NO: 383);
e. the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388), wherein percent identity is across the full length of SEQ ID NOs: 384-388;
f. the cleavable linker comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388);
g. the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), wherein percent identity is across the full length of SEQ ID NOs: 389-391; and/or
h. the Nef linking sequence comprises or consists of an amino acid sequence selected from VHAGPIA (SEQ ID NO: 389), VHAGPVA (SEQ ID NO: 390) and GALDI (SEQ ID NO: 391).

55. The expression vector of any one of claims 48 to 54, wherein the promoter sequence comprises a polynucleotide sequence of SEQ ID NO: 529.

56. A viral vector comprising the SAM of any one of claims 1 to 7, the expression cassette of any one of claims 8 to 10, or the expression vector of any one of claims 23 to 55.

57. The viral vector of claim 56, wherein:
a. the viral vector is derived from or based on a DNA virus or an RNA virus;
b. the viral vector is replication defective, replication deficient, replication attenuated or replication competent;
c. the viral vector is from a virus selected from adenovirus, adeno-associated virus, arenavirus, alphavirus, poxvirus, cytomegalovirus, rhabdovirus, vesicular stomatitis virus, flavivirus, maraba virus and vaccinia virus;
d. the viral vector is derived from or based on a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Flaviviridae (e.g. Yellow fever virus), Rhabdoviridae (e.g. Vesiculovirus, e.g. Maraba vesiculovirus), Togaviridae (e.g., Alphavirus, e.g., Venezuelan equine encephalitis virus);
e. the viral vector is derived from or based on an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV); or
f. the viral vector is derived from or based on an arenavirus vector selected from Lymphocytic choriomeningitis mammarenavirus (LCMV) or Cali mammarenavirus (a.k.a. Pichinde mammarenavirus or Pichinde arenavirus).

58. The viral vector of claim 57, wherein the arenavirus vector comprises a bi-segmented genome or a tri-segmented genome.

59. The viral vector of any one of claim 56 or 57, wherein the viral vector is a human adenovirus or a simian adenovirus.

60. The viral vector of claim 59, wherein the simian adenovirus is selected from a chimpanzee adenovirus, a gorilla adenovirus, and a rhesus adenovirus.

61. The viral vector of any one of claims 56, 57, and 59, wherein the viral vector is derived from or based on an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (ChAd), gorilla adenovirus, and rhesus adenovirus.

62. The viral vector of claim 61, wherein:
a. the ChAd is selected from ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), and ChAd147 (AdC147);
b. the gorilla adenovirus is selected from GC44, GC45, and GC46;
c. the rhesus adenovirus (RhAd) is selected from RhAd51, RhAd52, RhAd53, RhAd54, RhAd55, RhAd56, RhAd57, RhAd58, RhAd59, RhAd60, RhAd61, RhAd62, RhAd63, RhAd64, RhAd65 and RhAd66.

63. The viral vector of claim 62, wherein the ChAd is ChAd68.

64. The viral vector of any one of claims 59 to 63, wherein the human adenovirus or simian adenovirus comprises a modification of one or more adenoviral genes.

65. The viral vector of claim 64, wherein the one or more adenoviral genes are selected from E1, E3, and E4ORF2-4.

66. The viral vector of claim 64 or 65, wherein:
a. the modification comprises a deletion, substitution, or insertion of one or more nucleotides into the one or more adenoviral genes; or
b. the human adenovirus or simian adenovirus comprises a deletion of at least a portion of the E1 region.

67. The viral vector of claim 66.a, wherein the polynucleotide is inserted into the deletion of at least a portion of the E1 region.

68. The viral vector of any one of claims 56 to 67, wherein the viral vector comprises any one of the nucleic acid sequences of SEQ ID NOs: 520-521.

69. The viral vector of any one of claims 56 to 67, wherein the vector comprises a nucleic acid sequence that is at least 80%, 85%, 86%, 87%, 88%, 89%, 90%, 91%, 92%, 93%, 94%, 95%, 96%, 97%, 98%, 99%, or 100% identical to any one of the nucleic acid sequences of SEQ ID NOs: 520-521, wherein percent identity is across the full length of the nucleic acid sequence of SEQ ID NOs: 520-521.

70. A host cell comprising one or more SAMs of any one of claims 1 to 7, one or more expression cassettes of any one of claims 8 to 11, one or more LNPs of any one of claims 12 to 14, one or more PNPs of any one of claims 15 to 19, one or more nanoemulsions of any one of claims 20 to 22, one or more expression vectors of any one of claims 23 to 55, or one or more viral vectors of any one of claims 56 to 69.

71. The host cell of claim 70, wherein the one or more polynucleotides are not integrated into the host cell genome, e.g., are episomal.

72. The host cell of claim 70, wherein the one or more polynucleotides are integrated into the host cell genome.

73. The host cell of any one of claims 70 to 72, wherein the host cell is a mammalian cell.

74. The host cell of claim 73, wherein the mammalian cell is a human cell.

75. The host cell of claim 73, wherein the mammalian cell is not a human cell.

76. The host cell of any one of claims 70 to 75, wherein the host cell is in vitro.

77. The host cell of any one of claims 70 to 75, wherein the host cell is in vivo.

78. A polypeptide comprising:
a. any of the HIV immunogen amino acid sequences of SEQ ID NOs: 527-528; or
b. an amino nucleic acid sequence that is at least 80%, 85%, 86%, 87%, 88%, 89%, 90%, 91%, 92%, 93%, 94%, 95%, 96%, 97%, 98%, 99%, or 100% identical any of the HIV immunogen amino acid sequences of SEQ ID NOs: 527-528, wherein percent identity is across the full length of the HIV immunogen amino acid sequence of SEQ ID NOs: 527-528.

79. A composition comprising (a) one or more SAMs of any one of claims 1 to 7, one or more expression cassettes of any one of claims 8 to 11, one or more LNPs of any one of claims 12 to 14, one or more PNPs of any one of claims 15 to 19, one or more nanoemulsions of any one of claims 20 to 22, one or more expression vectors of any one of claims 23 to 55, one or more viral vectors of any one of claims 56 to 69, or one or more polypeptides of claim 78; and (b) a pharmaceutically acceptable diluent, carrier or excipient.

80. The composition of claim 79, comprising two or more SAMs of any one of claims 1 to 7, two or more expression cassettes of any one of claims 8 to 11, two or more LNPs of any one of claims 12 to 14, two or more PNPs of any one of claims 15 to 19, two or more nanoemulsions of any one of claims 20 to 22, or two or more expression vectors of any one of claims 23 to 55, two or more viral vectors of any one of claims 56 to 69, or two or more polypeptides of claim 78.

81. A composition comprising (a) one or more SAMs of any one of claims 1 to 7, one or more expression cassettes of any one of claims 8 to 11, one or more LNPs of any one of claims 12 to 14, one or more PNPs of any one of claims 15 to 19, or one or more nanoemulsions of any one of claims 20 to 22; and (b) one or more expression vectors of any one of claims 23 to 55 or one or more viral vectors of any one of claims 56 to 69.

82. A composition comprising (a) one or more polypeptides of claim 78; and (b) a pharmaceutically acceptable diluent, carrier or excipient.

83. The composition of claim 82, comprising two or more polypeptides of claim 78.

84. The composition of claim 81, further comprising a pharmaceutically acceptable diluent, carrier or excipient.

85. The composition of any one of claims 79 to 84, further comprising one or more of an adjuvant, an immunostimulator, a detergent, a micelle-forming agent, and an oil.

86. The composition of claim 85, wherein the immunostimulator is selected from a toll-like receptor (TLR) agonist, a cytokine, a non-coding immunostimulatory polynucleotide, an inhibitor of an inhibitory immune checkpoint protein or a stimulator of a stimulatory immune checkpoint protein.

87. The composition of claim 86, wherein:
a. the cytokine selected from IL-2, IL-7, IL-12, IL-15, IL-18, IL-21, IFN-a, IFN-γ, GM-CSF, FLT3LG, and combinations and functional variants thereof; or
b. the non-coding immunostimulatory polynucleotide is selected from a pathogen-activated molecular pattern (PAMP), a cytosine-phosphate-guanosine (CpG) oligodeoxynucleotide, and an immunostimulatory RNA (isRNA, e.g., CV8102).

88. The composition of any one of claims 79 to 87, wherein the composition is formulated for administration via a route selected from the group consisting of intravenous, intramuscular, intradermal, subcutaneous and mucosal (e.g., buccal, intranasal, intrarectal, intravaginal).

89. The composition of any one of claims 79 to 87.a, wherein the composition is formulated as a liquid.

90. The composition of any one of claims 79 to 87.a, wherein the composition is lyophilized.

91. A kit comprising one or more components selected from one or more SAMs of any one of claims 1 to 7, one or more expression cassettes of any one of claims 8 to 11, one or more LNPs of any one of claims 12 to 14, one or more PNPs of any one of claims 15 to 19, one or more nanoemulsions of any one of claims 20 to 22, one or more expression vectors of any one of claims 23 to 55, or one or more viral vectors of any one of claims 56 to 69, or one or more polypeptides of claim 78.

92. A kit comprising (a) a first component selected from one or more SAMs of any one of claims 1 to 7, one or more expression cassettes of any one of claims 8 to 11, one or more LNPs of any one of claims 12 to 14, one or more PNPs of any one of claims 15 to 19, or one or more nanoemulsions of any one of claims 20 to 22; and (b) second component comprising one or more expression vectors of any one of claims 23 to 55 or one or more viral vectors of any one of claims 56 to 69.

93. A kit comprising one or more unitary doses of one or more SAMs of any one of claims 1 to 7, one or more expression cassettes of any one of claims 8 to 11, one or more LNPs of any one of claims 12 to 14, one or more PNPs of any one of claims 15 to 19, one or more nanoemulsions of any one of claims 20 to 22, one or more expression vectors of any one of claims 23 to 55, or one or more viral vectors of any one of claims 56 to 69, or one or more polypeptides of claim 78.

94. A kit comprising (a) one or more unitary doses of one or more SAMs of any one of claims 1 to 7, one or more expression cassettes of any one of claims 8 to 11, one or more LNPs of any one of claims 12 to 14, one or more PNPs of any one of claims 15 to 19, or one or more nanoemulsions of any one of claims 20 to 22; and (b) one or more unitary doses of one or more expression vectors of any one of claims 23 to 55 or one or more viral vectors of any one of claims 56 to 69.

95. A kit comprising one or more unitary doses of one or more polypeptides of claim 78.

96. The kit of claim 91, wherein:
a. the one or more components are in a single container;
b. the one or more components are in two or more separate containers;
c. the first and second components are in a single container; or
d. the first and second components are in two or more separate containers.

97. The kit of claim 93 or 95, wherein:
a. the one or more unitary doses are in a single container;
b. the one or more unitary doses are in two or more separate containers.

98. The kit of any one of claims 96 to 97, comprising one or more containers selected from the group consisting of vials, ampules and pre-loaded syringes.

99. The kit of any one of claims 96 to 98, comprising one or more containers comprising the one or more SAMs, one or more LNPs, one or more PNPs, one or more nanoemulsions, one or more vectors, or one or more polypeptides in an aqueous solution.

100. The kit of any one of claims 93, 94, 95, and 97 to 99, wherein: (a) the one or more unitary doses are the same; or (b) the one or more unitary doses are the different.

101. The kit of any one of claims 93, 94, 95, and 97 to 100, comprising one or more unitary doses of one or more viral vectors of any one of claims 23 to 47, wherein the unitary doses are in the range of about 10³ to about 10¹⁵ viral focus forming units (FFU) or plaque forming units (PFU) or infectious units (IU) or viral particles (vp).

102. The kit of claim 101, wherein the unitary doses of the one or more viral vectors is from about 10⁴ to about 10⁷ viral FFU or PFU or IU or vp.

103. The kit of claim 101, wherein the unitary doses of the one or more viral vectors is from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp.

104. The kit of any one of claims 93, 94, and 97 to 100, comprising one or more unitary doses of one or more SAMs of any one of claims 1 to 7, wherein the unitary doses are in the range of about 1 μg to about 1000 μg.

105. The kit of claim 104, wherein the unitary doses of the one or more SAMs is from about 15 μg to about 500 μg.

106. The kit of claim 104, wherein the unitary doses of the one or more SAMs is from about 1 μg to about 100, 200, 300, 400, 500, 600, 700, 800, 900, or 1000 μg.

107. The composition of any one of claims 79 to 90 or the kit of any one of claims 91 to 106, further comprising one or more unitary doses of one or more additional therapeutic agents.

108. The composition or kit of claim 107, wherein the one or more additional therapeutic agents is selected from one or more agents that activate latent HIV, one or more agonists or activators of one or more toll-like receptors (TLRs), one or more interleukin receptor agonists, one or more cytokines, one or more receptor agonists, one or more inhibitors of a T-cell inhibitory immune checkpoint protein or receptor, one or more agonists, activators or stimulator of a T-cell stimulatory immune checkpoint protein or receptor, one or more inhibitors of CD47, one or more immune-based therapies, one or more broadly neutralizing antibodies (bnAbs), and one or more antiviral agents.

109. The composition or kit of claim 108, wherein:
a. the one or more agents that activate latent HIV is one or more latency reversing agents (LRAs);
b. the TLR agonist or activator is selected from the group consisting of a TLR2 agonist, a TLR3 agonist, a TLR4 agonist, a TLR5 agonist, a TLR7 agonist, a TLR8 agonist and a TLR9 agonist;

110. The composition or kit of claim 109, wherein the one or more LRAs is selected from the group consisting of agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics.

111. The composition or kit of claim 109, wherein:
a. the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist); or
b. the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042.

112. The composition or kit of claim 111, further comprising GS 9620 (vesatolimod).

113. The composition or kit of claim 111, further comprising lefitolimod (MGN-1703).

114. The composition or kit of claim 108, wherein:
a. the interleukin receptor agonist is an agonist of an interleukin receptor of an interleukin is selected from IL-2, IL-7, IL-12, IL-15, IL-18, IL-21, IFN-a, IFN-g, GM-CSF, FLT3LG;
b. the one or more cytokines selected from the group consisting of IL-2, IL-7, IL-12, IL-15, IL-18, IL-21, IFN-a, IFN-g, GM-CSF, FLT3LG, and combinations and functional variants thereof;
c. the receptor agonist is an agonist of one or more receptors selected from fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2);
d. the one or more inhibitors of a T-cell inhibitory immune checkpoint protein or receptor inhibits a checkpoint protein or receptor selected from the group consisting of CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1);
e. the one or more agonists, activators or stimulators of a T-cell stimulatory immune checkpoint protein or receptor agonizes, activates or stimulates a T-cell stimulatory immune checkpoint protein or receptor selected from the group consisting of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155);
f. the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors; or
g. the one or more immune-based therapies is selected from the group consisting of interferon alfa, interferon alfa-2b, interferon alfa-n3, pegylated interferon alfa, interferon gamma, fms related tyrosine kinase 3 (FLT3) agonists, gepon, normferon, peginterferon alfa-2a, peginterferon alfa-2b, and RPI-MN

115. The composition or kit of claim 114, wherein the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002.

116. The composition or kit of claim 108, 114, or 115, further comprising ipilimumab.

117. The composition or kit of claim 114, wherein the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 ( ) JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181.

118. The composition or kit of claim 108, 114, or 117, further comprising pembrolizumab.

119. The composition or kit of claim 108, 114, or 117, further comprising nivolumab.

120. The composition or kit of claim 108, 114, or 117, further comprising AB122 (zimberelimab).

121. The composition or kit of claim 114, wherein the FLT3 agonist is selected from GS-3583 and CDX-301.

122. The composition or kit of claim 108, 114, or 121, further comprising GS-3583.

123. The composition or kit of claim 108, 114, or 121, further comprising CDX-301.

124. The composition or kit of claim 108, further comprising one or more bnAbs.

125. The composition or kit of claim 108, wherein the one or more bnAbs is an HIV bnAb.

126. The composition or kit of claim 125, wherein the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses.

127. The composition or kit of any one of claims 108 and 124 to 126, wherein the bnAb is directed to the surface of Env: the CD4 binding site (CD4bs), the N-glycans associated with the V1/V2 and V3 loops, the silent face of gp120, the membrane proximal external region (MPER) of gp41, or a larger site spanning the interface between gp41 and gp120.

128. The composition or kit of any one of claims 108 and 124 to 127, wherein the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03, AND Z13.

129. The composition or kit of any one of claims 108 and 124 to 128, wherein the bnAb is an anti-CD4 bnAb.

130. The composition or kit of claim 129, wherein the anti-CD4 bnAb is selected from 3BNC117 and VRC01.

131. The composition or kit of any one of claims 108 and 124 to 128, wherein the bnAb is an anti-V3 bnAb.

132. The composition or kit of claim 131, wherein the anti-V3 bnAb is 10-1074.

133. A method for eliciting an immune response to human immunodeficiency virus (HIV) in a subject in need thereof, comprising administering to the subject one or more SAMs of any one of claims 1 to 7, one or more expression cassettes of any one of claims 8 to 11, one or more LNPs of any one of claims 12 to 14, one or more PNPs of any one of claims 15 to 19, one or more nanoemulsions of any one of claims 20 to 22, one or more expression vectors of any one of claims 23 to 55, one or more viral vectors of any one of claims 56 to 69, one or more polypeptides of claim 78, or one or more compositions of any one of claims 79 to 90.

134. A method of treating or preventing human immunodeficiency virus (HIV) in a subject in need thereof, comprising administering to the subject one or more SAMs of any one of claims 1 to 7, one or more expression cassettes of any one of claims 8 to 11, one or more LNPs of any one of claims 12 to 14, one or more PNPs of any one of claims 15 to 19, one or more nanoemulsions of any one of claims 20 to 22, one or more expression vectors of any one of claims 23 to 55, one or more viral vectors of any one of claims 56 to 69, one or more polypeptides of claim 78, or one or more compositions of any one of claims 79 to 90.

135. A method for eliciting an immune response to human immunodeficiency virus (HIV) in a subject in need thereof, comprising administering to the subject (a) one or more SAMs of any one of claims 1 to 7, one or more expression cassettes of any one of claims 8 to 11, one or more LNPs of any one of claims 12 to 14, one or more PNPs of any one of claims 15 to 19, one or more nanoemulsions of any one of claims 20 to 22; and (b) one or more expression vectors of any one of claims 23 to 55 or one or more viral vectors of any one of claims 56 to 69.

136. A method of treating or preventing human immunodeficiency virus (HIV) in a subject in need thereof, comprising administering to the subject (a) one or more SAMs of any one of claims 1 to 7, one or more expression cassettes of any one of claims 8 to 11, one or more LNPs of any one of claims 12 to 14, one or more PNPs of any one of claims 15 to 19, one or more nanoemulsions of any one of claims 20 to 22; and (b) one or more expression vectors of any one of claims 23 to 55 or one or more viral vectors of any one of claims 56 to 69.

137. The method of any one of claims 133 to 136, wherein:
a. the subject is infected with HIV-1, is suspected of being infected with HIV-1, or is at risk of being infected with HIV-1;
b. the subject is chronically infected with HIV-1;
c. the subject is acutely infected with HIV-1; or
d. the subject has an HIV-1 infection of Fiebig stage IV or earlier, e.g., Fiebig stage III, Fiebig stage II or Fiebig stage I.

138. The method of any one of claims 133 to 137, wherein administering comprises a route of administration selected from intravenous, intramuscular, intradermal, subcutaneous and mucosal (e.g. buccal, intranasal, intrarectal, intravaginal).

139. The method of any one of claims 133 to 138, wherein the method comprises administering one or more viral vectors of any one of claims 23 to 47 at a dose range from about 103 to about 1015 viral focus forming units (FFU) or plaque forming units (PFU) or infectious units (IU) or viral particles (vp), per administration.

140. The method of claim 139, wherein the one or more viral vectors is administered at a dose range from about 10⁴ to about 10⁷ viral FFU or PFU or IU or vp, e.g., from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration.

141. The method of any one of claims 133 to 140, wherein the method comprises administering one or more SAMs of any one of claims 1 to 7, one or more LNPs of any one of claims 12 to 14, one or more PNPs of any one of claims 15 to 19, one or more nanoemulsions of any one of claims 20 to 22 at a dose range from about 1 μg to about 1000 μg.

142. The method of any one of claims 133 to 141, wherein the one or more SAMs, LNPs, PNPs, or nanoemulsions is administered at a dose range from about 15 μg to about 500 μg.

143. The method of any one of claims 133 to 142, wherein the one or more SAMs, LNPs, PNPs, or nanoemulsions is administered at a dose range from about 1 μg to about 100, 200, 300, 400, 500, 600, 700, 800, 900, or 1000 μg.

144. The method of any one of claims 133 to 143, comprising a prime-boost regimen comprising:
(i) administering a priming composition at a first time point and administering one or more boosting compositions at one or more subsequent time points (e.g., prime-boost-boost-boost, etc.);
(ii) one or more iterations of administering a priming composition at a first time point and administering a boosting composition at a second time point (e.g., prime-boost-prime-boost, etc.); or
(iii) one more iterations of administering a priming composition at a first time point, administering a boosting composition at one or more subsequent time points, administering the priming composition at a subsequent time point after administration of the boosting composition (e.g., prime-boost-boost-prime, etc.).

145. The method of claim 144, wherein the administrations of the priming composition and the one or more boosting compositions are spaced at least 1 week, 2 weeks, 3 weeks or 1 month apart, e.g., at least 2, 3, 4, 5 or 6 months, apart.

146. The method of any one of claims 133 to 145, wherein:
a. the priming composition and the boosting composition comprise a polynucleotide encoding the same immunogenic polypeptide;
b. the priming composition and the boosting composition comprise polynucleotide encoding the different immunogenic polypeptides;
c. the priming composition and the boosting composition comprise the same one or more polypeptides, polynucleotides, SAMs, LNPs, PNPs, nanoemulsions, or viral vectors; or
d. the priming composition and the boosting composition comprise different polypeptides, polynucleotides, SAMs, LNPs, PNPs, nanoemulsions, or viral vectors.

147. The method of claim 146, comprising priming with a first polynucleotide, SAM, LNP, PNP, nanoemulsion, or viral vector, and boosting with a second polynucleotide, SAM, LNP, PNP, nanoemulsion, or viral vector.

148. The method of any one of claims 133 to 147, wherein the prime-boost regimen comprises:
a. Priming with a viral vector and boosting with a polynucleotide, wherein the polynucleotide is DNA, cDNA, mRNA or self-replicating RNA;
b. Priming with a polynucleotide, wherein the polynucleotide is DNA, cDNA, mRNA or self-replicating RNA, and boosting with a viral vector;
c. Priming with a first viral vector and boosting with a second viral vector, wherein the first and second viral vectors are from identical, related or unrelated taxonomical families;
d. Priming with a first replication deficient viral vector and boosting with a second replication deficient viral vector, wherein the first and second replication deficient viral expression vectors are from identical, related or unrelated taxonomical families;
e. Priming with a first attenuated deficient viral vector and boosting with a second replication attenuated viral vector, wherein the first and second replication attenuated viral vectors are from identical, related or unrelated taxonomical families;
f. Priming with a replication deficient viral vector and boosting with a replication attenuated viral vector;
g. Priming with a replication attenuated viral expression vector and boosting with a replication deficient viral vector;
h. Priming with a viral vector and boosting with a composition comprising a SAM;
i. Priming with an adenovirus viral vector and boosting with a polynucleotide, wherein the polynucleotide is DNA, cDNA, mRNA or self-replicating RNA;
j. Priming with an adenoviral vector and boosting with a composition comprising a SAM;
k. Priming with a ChAd vector and boosting with a polynucleotide, wherein the polynucleotide is DNA, cDNA, mRNA or self-replicating RNA; or
l. Priming with a ChAd vector and boosting with a composition comprising a SAM.

149. The method of any one of claims 133 to 148, wherein the subject is not receiving antiretroviral therapy (ART) or ART is discontinued prior to administration of the one or more polynucleotides, polypeptides, SAMs, LNPs, PNPs, nanoemulsions, expression cassettes, or viral vectors.

150. The method of any one of claims 133 to 149, wherein ART is discontinued after one or more administrations of the polynucleotides, polypeptides, SAMs, LNPs, PNPs, nanoemulsions, expression cassettes, or viral vectors.

151. The method of any one of claims 133 to 150, further comprising administering to the subject one or more additional therapeutic agents, e.g. two, three, four, or more additional therapeutic agents.

152. The method of claim 151, wherein the one or more therapeutic agents are selected from one or more agents that activate latent HIV, one or more toll-like receptors (TLRs) agonists or activators, one or more interleukin receptor agonists, one or more cytokines, one or more receptor agonists, one or more inhibitors of a T-cell inhibitory immune checkpoint protein or receptor, one or more agonists, activators or stimulators of a T-cell stimulatory immune checkpoint protein or receptor, one or more CD47 inhibitors, one or more anti-viral agents, one or more immune-based therapies, and one or more broadly neutralizing antibodies (bnAbs).

153. The method of claim 152, wherein:
a. the one or more agents that activate latent HIV is one or more latency reversing agents (LRAs);
b. the one or more TLR agonists or activators is selected from the group consisting of a TLR2 agonist, a TLR3 agonist, a TLR4 agonist, a TLR5 agonist, a TLR7 agonist, a TLR8 agonist and a TLR9 agonist;
c. the one or more cytokines is selected from the group consisting of IL-2, IL-7, IL-12, IL-15, IL-18, IL-21, IFN-a, IFN-g, GM-CSF, FLT3LG, and combinations and functional variants thereof;
d. the one or more interleukin receptor agonists is an agonist of an interleukin selected from IL-2, IL-7, IL-12, IL-15, IL-18, IL-21, IFN-a, IFN-g, GM-CSF and FLT3LG;
e. the one or more receptor agonists is an agonist of a receptor selected from the group consisting of fms related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2); the one or more inhibitors of a T-cell inhibitory immune checkpoint protein or receptor is an inhibitor of a T-cell inhibitory immune checkpoint protein or receptor selected from CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1);
f. the one or more agonists, activators or stimulators of a T-cell stimulatory immune checkpoint protein or receptor agonizes, activates or stimulates a T-cell stimulatory immune checkpoint protein or receptor selected from of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155);
g. the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors; or
h. the one or more immune-based therapies is selected from the group consisting of interferon alfa, interferon alfa-2b, interferon alfa-n3, pegylated interferon alfa, interferon gamma; fms related tyrosine kinase 3 (FLT3) agonists, gepon, normferon, peginterferon alfa-2a, peginterferon alfa-2b, and RPI-MN

154. The method of claim 153, wherein the one or more LRAs are selected from agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics.

155. The method of claim 153, wherein the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist).

156. The method of claim 155, further comprising administering GS 9620 (vesatolimod) to the subject.

157. The method of claim 153, wherein the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042.

158. The method of claim 157, further comprising administering lefitolimod (MGN-1703) to the subject.

159. The method of any one of claims 152 to 158, further comprising administering one or more bnAbs to the subject.

160. The method of claim 152 or 159, wherein the one or more bnAbs is an HIV bnAb.

161. The method of claim 160, wherein the HIV bnAb is highly effective against most circulating strains of HIV, neutralises a wide range of genetically diverse HIV-1 subtypes, and/or potently neutralises a substantial percentage of primary isolates or exhibit some capacity to reach across clades and harder to neutralise tier 2 and 3 viruses.

162. The method of any one of claims 152 and 159 to 161, wherein the bnAb is directed to the surface of Env, the CD4 binding site (CD4bs), the N-glycans associated with the V1/V2 and V3 loops, the silent face of gp120, the membrane proximal external region (MPER) of gp41, or a larger site spanning the interface between gp41 and gp120.

163. The method of any one of claims 152 and 159 to 162, wherein the bnAb is selected from 10-1074, 10E8, 12A12, 12A21, 2F5, 2G12, 3BC176, 3BNC117, 3BNC55, 3BNC60, 3BNC62, 447-52D, 4E10, 5H/11-BMV-D5, 8ANC195, b12, CH01, CH02, CH03, CH04, CH103, HGN194, HJ16, HK20, M66.6, NIH45-46, PG16, PG9, PGT121, PGT122, PGT123, PGT125, PGT126, PGT127, PGT128, PGT130, PGT131, PGT135, PGT136, PGT137, PGT141, PGT142, PGT143, PGT144, PGT145, VRC-CH30, CRC-CH31, CRC-CH32, CRC-CH33, CRC-CH34, VRC-PG04, VRC-PG04b, VRC01, VRC02, VRC03,

164. The method of any one of claims 152 and 159 to 163, wherein the bnAb is an anti-CD4 bnAb.

165. The method of claim 164, wherein the anti-CD4 bnAb is selected from 3BNC117 and VRC01.

166. The method of any one of claims 152 and 159 to 163, wherein the bnAb is an anti-V3 bnAb.

167. The method of claim 166, wherein the anti-V3 bnAb is 10-1074.

168. The method of claim 153, wherein:
a. the inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), AK-104 (CTLA4/PD-1) and BPI-002;
b. the inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFb-EC domain), CA-170 (PD-L1/VISTA), CDX-527 (CD27/PD-L1), LY-3415244 (TIM3/PDL1), INBRX-105 (4-1BB/PDL1), GS-4224, GS-4416, INCB086550 and MAX10181;
c. the FLT3 agonist is selected from GS-3583 and CDX-301.

169. The method of claim 152, 153 or 168, further comprising administering ipilimumab to the subject.

170. The method of claim 152, 153 or 168, further comprising administering pembrolizumab to the subject.

171. The method of claim 152, 153 or 168, further comprising administering nivolumab, to the subject.

172. The method of claim 153 or 168, further comprising administering AB122 (zimberelimab) to the subject.

173. The method of claim 152, 153 or 168, wherein the method further comprises administering GS-3583 to the subject.

174. The method of claim 152, 153 or 168, wherein the method further comprises administering CDX-301 to the subject.

175. The method of any one of claims 133 to 174, wherein after one or more administrations of one or more of polynucleotides, polypeptides, SAMs, LNPs, PNPs, microemulsions, expression cassettes, or viral vectors, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more.

176. The method of any one of claims 133 to 175, wherein, after one or more administrations of one or more of polynucleotides, polypeptides, SAMs, LNPs, PNPs, microemulsions, expression cassettes, or viral vectors, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

177. One or more SAMs of any one of claims 1 to 7, one or more expression cassettes of any one of claims 8 to 11, one or more LNPs of any one of claims 12 to 14, one or more PNPs of any one of claims 15 to 19, one or more nanoemulsions of any one of claims 20 to 22, one or more expression vectors of any one of claims 23 to 55, one or more viral vectors of any one of claims 56 to 69, one or more polypeptides of claim 78 or 78.a, or one or more compositions of any one of claims 79 to 90, for use in eliciting an immune response to human immunodeficiency virus (HIV) in a subject in need thereof.

178. One or more SAMs of any one of claims 1 to 7, one or more expression cassettes of any one of claims 8 to 11, one or more LNPs of any one of claims 12 to 14, one or more PNPs of any one of claims 15 to 19, one or more nanoemulsions of any one of claims 20 to 22, one or more expression vectors of any one of claims 23 to 55, one or more viral vectors of any one of claims 56 to 69, one or more polypeptides of claim 78, or one or more compositions of any one of claims 79 to 90, for use in treating or preventing human immunodeficiency virus (HIV) in a subject in need thereof.

179. One or more SAMs of any one of claims 1 to 7, one or more expression cassettes of any one of claims 8 to 11, one or more LNPs of any one of claims 12 to 14, one or more PNPs of any one of claims 15 to 19, one or more nanoemulsions of any one of claims 20 to 22, one or more expression vectors of any one of claims 23 to 55, one or more viral vectors of any one of claims 56 to 69, one or more polypeptides of claim 78, or one or more compositions of any one of claims 79 to 90, for use as a medicament.


SEQUENCE LISTING (538 sequences)

SEQ ID NO: 1 - protein, 25 aa, synthetic construct
CSATEKLWVTVYYGVPVWKEATTTL

SEQ ID NO: 2 - protein, 15 aa, synthetic construct
LWVTVYYGVPVWKEA

SEQ ID NO: 3 - protein, 15 aa, synthetic construct
LWVTIYYGVPVWKDA

SEQ ID NO: 4 - protein, 14 aa, synthetic construct
LWVTVYYGVPVWKE

SEQ ID NO: 5 - protein, 14 aa, synthetic construct
LWVTIYYGVPVWKD

SEQ ID NO: 6 - protein, 9 aa, synthetic construct
VTVYYGVPV

SEQ ID NO: 7 - protein, 9 aa, synthetic construct
VTIYYGVPV

SEQ ID NO: 8 - protein, 14 aa, synthetic construct
ATTTLFCASDAKAY

SEQ ID NO: 9 - protein, 14 aa, synthetic construct
ANTTLFCASDAKGY

SEQ ID NO: 10 - protein, 25 aa, synthetic construct
KAYDTEVHNVWATHACVPTDPNPQE

SEQ ID NO: 11 - protein, 19 aa, synthetic construct
AHNVWATHACVPTDPNPQE

SEQ ID NO: 12 - protein, 19 aa, synthetic construct
VHNIWATHACVPTDPSPQE

SEQ ID NO: 13 - protein, 18 aa, synthetic construct
HNVWATHACVPTDPNPQE

SEQ ID NO: 14 - protein, 18 aa, synthetic construct
HNIWATHACVPTDPSPQE

SEQ ID NO: 15 - protein, 9 aa, synthetic construct
NVWATHACV

SEQ ID NO: 16 - protein, 9 aa, synthetic construct
NIWATHACV

SEQ ID NO: 17 - protein, 23 aa, synthetic construct
DIISLWDQSLKPCVKLTPLCVTL

SEQ ID NO: 18 - protein, 23 aa, synthetic construct
DIISLWDESLKPCVKLTPICVTL

SEQ ID NO: 19 - protein, 25 aa, synthetic construct
DQSLKPCVKLTPLCVTLNCTDLRNT

SEQ ID NO: 20 - protein, 25 aa, synthetic construct
DESLKPCVKLTPICVTLNCTDLRNT

SEQ ID NO: 21 - protein, 9 aa, synthetic construct
KLTPLCVTL

SEQ ID NO: 22 - protein, 9 aa, synthetic construct
KLTPICVTL

SEQ ID NO: 23 - protein, 18 aa, synthetic construct
SFEPIPIHYCAPAGFAIL

SEQ ID NO: 24 - protein, 18 aa, synthetic construct
TFEPIPIHYCTPAGFAIL

SEQ ID NO: 25 - protein, 9 aa, synthetic construct
PAGFAILKC

SEQ ID NO: 26 - protein, 9 aa, synthetic construct
PAGFALLKC

SEQ ID NO: 27 - protein, 25 aa, synthetic construct
GTGPCTNVSTVQCTHGIRPVVSTQL

SEQ ID NO: 28 - protein, 28 aa, synthetic construct
NVSTVQCTHGIRPVVSTQLLLNGSLAEE

SEQ ID NO: 29 - protein, 28 aa, synthetic construct
NISTVQCTHGIKPVVSTQLLLNGSLAEK

SEQ ID NO: 30 - protein, 9 aa, synthetic construct
STVQCTHGI

SEQ ID NO: 31 - protein, 11 aa, synthetic construct
FNCGGEFFYCN

SEQ ID NO: 32 - protein, 11 aa, synthetic construct
FNCRGEFFYCN

SEQ ID NO: 33 - protein, 10 aa, synthetic construct
VGKAMYAPPI

SEQ ID NO: 34 - protein, 10 aa, synthetic construct
VGRAMYAPPI

SEQ ID NO: 35 - protein, 10 aa, synthetic construct
GGDMRDNWRS

SEQ ID NO: 36 - protein, 10 aa, synthetic construct
GGNMKDNWRS

SEQ ID NO: 37 - protein, 15 aa, synthetic construct
MRDNWRSELYKYKVV

SEQ ID NO: 38 - protein, 15 aa, synthetic construct
MKDNWRSELYRYKVV

SEQ ID NO: 39 - protein, 11 aa, synthetic construct
AKRRVVQREKR

SEQ ID NO: 40 - protein, 11 aa, synthetic construct
ARRRVVQREKR

SEQ ID NO: 41 - protein, 105 aa, synthetic construct
KRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTT

SEQ ID NO: 42 - protein, 105 aa, synthetic construct
RRRVVQREKRAIGLGAVFLGFLGTAGSTMGAASMTLTVQARLLLSGIVQQQSNLLRAIEAQQHMLQLTVWGIKQLQARILAVERYLRDQQLLGIWGCSGRLICTT

SEQ ID NO: 43 - protein, 16 aa, synthetic construct
FLGFLGAAGSTMGAAS

SEQ ID NO: 44 - protein, 16 aa, synthetic construct
FLGFLGTAGSTMGAAA

SEQ ID NO: 45 - protein, 74 aa, synthetic construct
ASITLTVQARQLLSGIVQQQNNLLRAIEAQQHLLQLTVWGIKQLQARVLAVERYLKDQQLLGIWGCSGKLICTT

SEQ ID NO: 46 - protein, 74 aa, synthetic construct
ASMTLTVQARLLLSGIVQQQSNLLRAIEAQQHMLQLTVWGIKQLQARILAVERYLRDQQLLGIWGCSGRLICTT

SEQ ID NO: 47 - protein, 21 aa, synthetic construct
TLTVQARQLLSGIVQQQNNLL

SEQ ID NO: 48 - protein, 21 aa, synthetic construct
TLTVQARLLLSGIVQQQSNLL

SEQ ID NO: 49 - protein, 11 aa, synthetic construct
NLLRAIEAQQH

SEQ ID NO: 50 - protein, 11 aa, synthetic construct
NLLKAIEAQQH

SEQ ID NO: 51 - protein, 27 aa, synthetic construct
AIEAQQHLLQLTVWGIKQLQARVLAVE

SEQ ID NO: 52 - protein, 27 aa, synthetic construct
AIEAQQHMLQLTVWGIKQLQARILAVE

SEQ ID NO: 53 - protein, 9 aa, synthetic construct
ERYLKDQQL

SEQ ID NO: 54 - protein, 9 aa, synthetic construct
ERYLRDQQL

SEQ ID NO: 55 - protein, 9 aa, synthetic construct
YLKDQQLLG

SEQ ID NO: 56 - protein, 9 aa, synthetic construct
YLRDQQLLG

SEQ ID NO: 57 - protein, 25 aa, synthetic construct
YLKDQQLLGIWGCSGKLICTTAVPW

SEQ ID NO: 58 - protein, 18 aa, synthetic construct
DQQLLGIWGCSGKLICTT

SEQ ID NO: 59 - protein, 18 aa, synthetic construct
DQQLLGLWGCSGKLICPT

SEQ ID NO: 60 - protein, 9 aa, synthetic construct
GIWGCSGKL

SEQ ID NO: 61 - protein, 9 aa, synthetic construct
GLWGCSGKL

SEQ ID NO: 62 - protein, 11 aa, synthetic construct
WLWYIKIFIMI

SEQ ID NO: 63 - protein, 11 aa, synthetic construct
WLWYIRIFIMI

SEQ ID NO: 64 - protein, 14 aa, synthetic construct
IFIMIVGGLIGLRI

SEQ ID NO: 65 - protein, 14 aa, synthetic construct
LFIMIVGGLVGLRI

SEQ ID NO: 66 - protein, 15 aa, synthetic construct
VNRVRQGYSPLSFQT

SEQ ID NO: 67 - protein, 15 aa, synthetic construct
VNRVRKGYSPLSFQI

SEQ ID NO: 68 - protein, 11 aa, synthetic construct
MGARASVLSGG

SEQ ID NO: 69 - protein, 11 aa, synthetic construct
MGARASILSGG

SEQ ID NO: 70 - protein, 53 aa, synthetic construct
MGARASVLSGGELDRWEKIRLRPGGKKKYRLKHIVWASRELERFAVNPGLLET

SEQ ID NO: 71 - protein, 53 aa, synthetic construct
MGARASILSGGKLDKWEKIRLRPGGRKKYKLKHIVWASRELERFAVNPGLLET

SEQ ID NO: 72 - protein, 13 aa, synthetic construct
LDRWEKIRLRPGG

SEQ ID NO: 73 - protein, 13 aa, synthetic construct
LDKWEKIRLRPMG

SEQ ID NO: 74 - protein, 9 aa, synthetic construct
IRLRPGGKK

SEQ ID NO: 75 - protein, 9 aa, synthetic construct
IRLRPGGRK

SEQ ID NO: 76 - protein, 23 aa, synthetic construct
LKHIVWASRELERFAVNPGLLET

SEQ ID NO: 77 - protein, 23 aa, synthetic construct
LKHLVWASRELERFALNPGLLET

SEQ ID NO: 78 - protein, 15 aa, synthetic construct
ASRELERFAVNPGLL

SEQ ID NO: 79 - protein, 15 aa, synthetic construct
ASRELERFALNPGLL

SEQ ID NO: 80 - protein, 9 aa, synthetic construct
TGSEELKSL

SEQ ID NO: 81 - protein, 9 aa, synthetic construct
TGSEELRSL

SEQ ID NO: 82 - protein, 9 aa, synthetic construct
DTKEALDKI

SEQ ID NO: 83 - protein, 9 aa, synthetic construct
DTKEALEKI

SEQ ID NO: 84 - protein, 9 aa, synthetic construct
EALDKIEEE

SEQ ID NO: 85 - protein, 9 aa, synthetic construct
EALEKIEEE

SEQ ID NO: 86 - protein, 10 aa, synthetic construct
VSQNYPIVQN

SEQ ID NO: 87 - protein, 10 aa, synthetic construct
VSQNFPIVQN

SEQ ID NO: 88 - protein, 231 aa, synthetic construct
PIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVL

SEQ ID NO: 89 - protein, 231 aa, synthetic construct
PIVQNIQGQMVHQPISPRTLNAWVKVIEEKAFSPEVIPMFTALSEGATPHDLNTMLNTIGGHQAAMQMLKDTINEEAAEWDRVHPVHAGPVAPGQMRDPRGSDIAGTTSNLQEQIGWMTSNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVL

SEQ ID NO: 90 - protein, 25 aa, synthetic construct
MVHQAISPRTLNAWVKVVEEKAFSP

SEQ ID NO: 91 - protein, 25 aa, synthetic construct
MVHQPISPRTLNAWVKVIEEKAFSP

SEQ ID NO: 92 - protein, 71 aa, synthetic construct
ISPRTLNAWVKVVEEKAFSPEVIPMFSALSEGATPQDLNTMLNTVGGHQAAMQMLKETINEEAAEWDRLHP

SEQ ID NO: 93 - protein, 71 aa, synthetic construct
LSPRTLNAWVKVIEEKAFSPEVIPMFTALSEGATPHDLNTMLNTIGGHQAAMQMLKDTINEEAAEWDRVHP

SEQ ID NO: 94 - protein, 223 aa, synthetic construct
ISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQ

SEQ ID NO: 95 - protein, 223 aa, synthetic construct
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQ

SEQ ID NO: 96 - protein, 9 aa, synthetic construct
RTLNAWVKV

SEQ ID NO: 97 - protein, 25 aa, synthetic construct
LSEGATPQDLNTMLNTVGGHQAAMQ

SEQ ID NO: 98 - protein, 25 aa, synthetic construct
LSEGATPHDLNTMLNTIGGHQAAMQ

SEQ ID NO: 99 - protein, 9 aa, synthetic construct
DLNTMLNTV

SEQ ID NO: 100 - protein, 9 aa, synthetic construct
DLNTMLNTI

SEQ ID NO: 101 - protein, 27 aa, synthetic construct
PGQMREPRGSDIAGTTSTLQEQIGWMT

SEQ ID NO: 102 - protein, 27 aa, synthetic construct
PGQMRDPRGSDIAGSTSTLQEQIAWMT

SEQ ID NO: 103 - protein, 33 aa, synthetic construct
NPPIPVGEIYKRWIILGLNKIVRMYSPTSILDI

SEQ ID NO: 104 - protein, 33 aa, synthetic construct
NPPIPVGDIYKRWIIMGLNKIVRMYSPVSILDI

SEQ ID NO: 105 - protein, 34 aa, synthetic construct
PVGEIYKRWIILGLNKIVRMYSPTSILDIRQGPK

SEQ ID NO: 106 - protein, 34 aa, synthetic construct
PVGDIYKRWIIMGLNKIVRMYSPVSILDIKQGPK

SEQ ID NO: 107 - protein, 18 aa, synthetic construct
WIILGLNKIVRMYSPTSI

SEQ ID NO: 108 - protein, 18 aa, synthetic construct
WIIMGLNKIVRMYSPVSI

SEQ ID NO: 109 - protein, 34 aa, synthetic construct
SILDIRQGPKEPFRDYVDRFYKTLRAEQASQEVK

SEQ ID NO: 110 - protein, 34 aa, synthetic construct
SILDIKQGPKEPFRDYVDRFYRTLRAEQASQDVK

SEQ ID NO: 111 - protein, 34 aa, synthetic construct
GPKEPFRDYVDRFYKTLRAEQASQEVKNWMTETL

SEQ ID NO: 112 - protein, 34 aa, synthetic construct
GPKEPFRDYVDRFYRTLRAEQASQDVKNWMTETL

SEQ ID NO: 113 - protein, 18 aa, synthetic construct
YVDRFYKTLRAEQASQEV

SEQ ID NO: 114 - protein, 18 aa, synthetic construct
YVDRFYRTLRAEQASQDV

SEQ ID NO: 115 - protein, 59 aa, synthetic construct
QEVKNWMTETLLVQNANPDCKTILKALGPAATLEEMMTACQGVGGPGHKARVLAEAMSQ

SEQ ID NO: 116 - protein, 59 aa, synthetic construct
QDVKNWMTETLLVQNSNPDCKTILKALGPGATLEEMMSACQGVGGPSHKARVLAEAMCQ

SEQ ID NO: 117 - protein, 25 aa, synthetic construct
ILKALGPAATLEEMMTACQGVGGPG

SEQ ID NO: 118 - protein, 25 aa, synthetic construct
ILKALGPGATLEEMMSACQGVGGPS

SEQ ID NO: 119 - protein, 25 aa, synthetic construct
LGPAATLEEMMTACQGVGGPGHKAR

SEQ ID NO: 120 - protein, 25 aa, synthetic construct
LGPGATLEEMMSACQGVGGPSHKAR

SEQ ID NO: 121 - protein, 9 aa, synthetic construct
ATLEEMMTA

SEQ ID NO: 122 - protein, 9 aa, synthetic construct
ATLEEMMSA

SEQ ID NO: 123 - protein, 9 aa, synthetic construct
EMMTACQGV

SEQ ID NO: 124 - protein, 9 aa, synthetic construct
EMMSACQGV

SEQ ID NO: 125 - protein, 10 aa, synthetic construct
KCFNCGKEGH

SEQ ID NO: 126 - protein, 10 aa, synthetic construct
KCFNCGREGH

SEQ ID NO: 127 - protein, 9 aa, synthetic construct
ARNCRAPRK

SEQ ID NO: 128 - protein, 9 aa, synthetic construct
AKNCRAPRK

SEQ ID NO: 129 - protein, 39 aa, synthetic construct
ARNCRAPRKKGCWKCGKEGHQMKDCTERQANFLGKIWPS

SEQ ID NO: 130 - protein, 39 aa, synthetic construct
AKNCRAPRKRGCWKCGREGHQMKDCNERQANFLGKVWPS

SEQ ID NO: 131 - protein, 14 aa, synthetic construct
NCRAPRKKGCWKCG

SEQ ID NO: 132 - protein, 14 aa, synthetic construct
NCRAPRKRGCWKCG

SEQ ID NO: 133 - protein, 19 aa, synthetic construct
GCWKCGKEGHQMKDCTERQ

SEQ ID NO: 134 - protein, 19 aa, synthetic construct
GCWKCGREGHQMKDCNERQ

SEQ ID NO: 135 - protein, 17 aa, synthetic construct
KDCTERQANFLGKIWPS

SEQ ID NO: 136 - protein, 17 aa, synthetic construct
KDCNERQANFLGKVWPS

SEQ ID NO: 137 - protein, 16 aa, synthetic construct
RQANFLGKIWPSHKGR

SEQ ID NO: 138 - protein, 16 aa, synthetic construct
RQANFLGKVWPSHNGR

SEQ ID NO: 139 - protein, 12 aa, synthetic construct
KGRPGNFLQSRP

SEQ ID NO: 140 - protein, 12 aa, synthetic construct
NGRPGNFLQNRP

SEQ ID NO: 141 - protein, 10 aa, synthetic construct
SLRSLFGNDP

SEQ ID NO: 142 - protein, 10 aa, synthetic construct
SLKSLFGNDP

SEQ ID NO: 143 - protein, 9 aa, synthetic construct
SLFGNDPSS

SEQ ID NO: 144 - protein, 9 aa, synthetic construct
SLFGNDPLS

SEQ ID NO: 145 - protein, 33 aa, synthetic construct
LKHIVWASRELERFAVNPGLLETVSQNYPIVQN

SEQ ID NO: 146 - protein, 33 aa, synthetic construct
LKHLVWASRELERFALNPGLLETVSQNFPIVQN

SEQ ID NO: 147 - protein, 9 aa, synthetic construct
GVGAVSRDL

SEQ ID NO: 148 - protein, 9 aa, synthetic construct
GVGAASRDL

SEQ ID NO: 149 - protein, 19 aa, synthetic construct
EEVGFPVRPQVPLRPMTYK

SEQ ID NO: 150 - protein, 19 aa, synthetic construct
EEVGFPVKPQVPLRPMTFK

SEQ ID NO: 151 - protein, 36 aa, synthetic construct
EEVGFPVKPQVPLRPMTFKGALDLSHFLREKGGLEG

SEQ ID NO: 152 - protein, 36 aa, synthetic construct
EEVGFPVRPQVPLRPMTYKGALDLSHFLKEKGGLEG

SEQ ID NO: 153 - protein, 22 aa, synthetic construct
YKAAVDLSHFLREKGGLEGAAY

SEQ ID NO: 154 - protein, 22 aa, synthetic construct
YKGALDLSHFLKEKGGLEGAAY

SEQ ID NO: 155 - protein, 10 aa, synthetic construct
SHFLKEKGGL

SEQ ID NO: 156 - protein, 10 aa, synthetic construct
SHFLREKGGL

SEQ ID NO: 157 - protein, 9 aa, synthetic construct
LKEKGGLEG

SEQ ID NO: 158 - protein, 9 aa, synthetic construct
LREKGGLEG

SEQ ID NO: 159 - protein, 16 aa, synthetic construct
TQGYFPDWQNYTPGPG

SEQ ID NO: 160 - protein, 16 aa, synthetic construct
TQGFFPDWQNYTPEPG

SEQ ID NO: 161 - protein, 32 aa, synthetic construct
TQGFFPDWQNYTPEPGIRFPLTFGWCFKLVPL

SEQ ID NO: 162 - protein, 32 aa, synthetic construct
TQGYFPDWQNYTPGPGTRYPLTFGWCFKLVPV

SEQ ID NO: 163 - protein, 19 aa, synthetic construct
EPGIRFPLTFGWCFKLVPL

SEQ ID NO: 164 - protein, 19 aa, synthetic construct
GPGTRYPLTFGWCFKLVPV

SEQ ID NO: 165 - protein, 25 aa, synthetic construct
GPGIRYPLLTFGWCFKLPVEPEKVE

SEQ ID NO: 166 - protein, 9 aa, synthetic construct
RYPLTFGWC

SEQ ID NO: 167 - protein, 9 aa, synthetic construct
RFPLTFGWC

SEQ ID NO: 168 - protein, 15 aa, synthetic construct
RYPLTFGWCFKLVPV

SEQ ID NO: 169 - protein, 15 aa, synthetic construct
RFPLTFGWCFKLVPL

SEQ ID NO: 170 - protein, 13 aa, synthetic construct
PLTFGWCFKLVPV

SEQ ID NO: 171 - protein, 13 aa, synthetic construct
PLCFGWCFKLVPL

SEQ ID NO: 172 - protein, 9 aa, synthetic construct
LTFGWCFKL

SEQ ID NO: 173 - protein, 9 aa, synthetic construct
LCFGWCFKL

SEQ ID NO: 174 - protein, 12 aa, synthetic construct
FPQITLWQRPLV

SEQ ID NO: 175 - protein, 12 aa, synthetic construct
LPQITLWQRPIV

SEQ ID NO: 176 - protein, 62 aa, synthetic construct
FPQITLWQRPLVTIKIGGQLKEALLDTGADDTVLEEMNLPGRWKPKMIGGIGGFIKVRQYDQ

SEQ ID NO: 177 - protein, 62 aa, synthetic construct
LPQITLWQRPIVTIKIGGQIKEALLDTGADDTVLEDMNLPGKWKPKMIGGIGGFIKVKQYDQ

SEQ ID NO: 178 - protein, 20 aa, synthetic construct
GGQLKEALLDTGADDTVLEE

SEQ ID NO: 179 - protein, 20 aa, synthetic construct
GGQIKEALLDTGADDTVLED

SEQ ID NO: 180 - protein, 24 aa, synthetic construct
LPGRWKPKMIGGIGGFIKVRQYDQ

SEQ ID NO: 181 - protein, 24 aa, synthetic construct
LPGKWKPKMIGGIGGFIKVKQYDQ

SEQ ID NO: 182 - protein, 132 aa, synthetic construct
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKS

SEQ ID NO: 183 - protein, 132 aa, synthetic construct
GTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKS

SEQ ID NO: 184 - protein, 149 aa, synthetic construct
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDK

SEQ ID NO: 185 - protein, 149 aa, synthetic construct
GTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTILDVGDAYFSIPLDK

SEQ ID NO: 186 - protein, 161 aa, synthetic construct
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPS

SEQ ID NO: 187 - protein, 161 aa, synthetic construct
GTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPS

SEQ ID NO: 188 - protein, 192 aa, synthetic construct
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 189 - protein, 192 aa, synthetic construct
GTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 190 - protein, 25 aa, synthetic construct
NLLTQIGCTLNFPISPIETVPVKLK

SEQ ID NO: 191 - protein, 25 aa, synthetic construct
NLLTQLGCTLNFPISPIDTVPVKLK

SEQ ID NO: 192 - protein, 9 aa, synthetic construct
TLNFPISPI

SEQ ID NO: 193 - protein, 24 aa, synthetic construct
GLKKKKSVTVLDVGDAYFSVPLDK

SEQ ID NO: 194 - protein, 24 aa, synthetic construct
GLKKNKSVTVLDVGDAYFSIPLDK

SEQ ID NO: 195 - protein, 12 aa, synthetic construct
DFRKYTAFTIPS

SEQ ID NO: 196 - protein, 12 aa, synthetic construct
EFRKYTAFTVPS

SEQ ID NO: 197 - protein, 25 aa, synthetic construct
NNETPGIRYQYNVLPQGWKGSPAIF

SEQ ID NO: 198 - protein, 25 aa, synthetic construct
NNETPGVRYQYNVLPMGWKGSPAIF

SEQ ID NO: 199 - protein, 30 aa, synthetic construct
NNETPGIRYQYNVLPQGWKGSPAIFQSSMT

SEQ ID NO: 200 - protein, 30 aa, synthetic construct
NNETPGVRYQYNVLPMGWKGSPAIFQCSMT

SEQ ID NO: 201 - protein, 9 aa, synthetic construct
YQYNVLPQG

SEQ ID NO: 202 - protein, 9 aa, synthetic construct
YQYNVLPMG

SEQ ID NO: 203 - protein, 9 aa, synthetic construct
FQSSMTKIL

SEQ ID NO: 204 - protein, 9 aa, synthetic construct
FQCSMTKIL

SEQ ID NO: 205 - protein, 10 aa, synthetic construct
SMTKILEPFR

SEQ ID NO: 206 - protein, 10 aa, synthetic construct
SMTKILDPFR

SEQ ID NO: 207 - protein, 9 aa, synthetic construct
ILEPFRKQN

SEQ ID NO: 208 - protein, 9 aa, synthetic construct
ILDPFRKQN

SEQ ID NO: 209 - protein, 25 aa, synthetic construct
FRKQNPDIVIYQYMDDLYVGSDLEI

SEQ ID NO: 210 - protein, 25 aa, synthetic construct
KQNPDIVIYQYMDDLYVGSDLEIGQ

SEQ ID NO: 211 - protein, 25 aa, synthetic construct
NPDIVIYQYMDDLYVGSDLEIGQHR

SEQ ID NO: 212 - protein, 25 aa, synthetic construct
NPDIVIYQYVDDLYVGSDLEIEQHR

SEQ ID NO: 213 - protein, 22 aa, synthetic construct
IVIYQYMDDLYVGSDLEIGQHR

SEQ ID NO: 214 - protein, 22 aa, synthetic construct
IVIYQYVDDLYVGSDLEIEQHR

SEQ ID NO: 215 - protein, 9 aa, synthetic construct
VIYQYMDDL

SEQ ID NO: 216 - protein, 9 aa, synthetic construct
VIYQYVDDL

SEQ ID NO: 217 - protein, 9 aa, synthetic construct
YQYMDDLYV

SEQ ID NO: 218 - protein, 9 aa, synthetic construct
YQYVDDLYV

SEQ ID NO: 219 - protein, 9 aa, synthetic construct
YMDDLYVGS

SEQ ID NO: 220 - protein, 9 aa, synthetic construct
YVDDLYVGS

SEQ ID NO: 221 - protein, 33 aa, synthetic construct
WGFTTPDKKHQKEPPFLWMGYELHPDKWTVQPI

SEQ ID NO: 222 - protein, 33 aa, synthetic construct
WGLTTPDKKHQKDPPFLWMGYELHPDRWTVQPI

SEQ ID NO: 223 - protein, 65 aa, synthetic construct
WGFTTPDKKHQKEPPFLWMGYELHPDKWTVQPIVLPEKDSWTVNDIQKLVGKLNWASQIYPGIKV

SEQ ID NO: 224 - protein, 65 aa, synthetic construct
WGLTTPDKKHQKDPPFLWMGYELHPDRWTVQPIELPEKESWTVNDIQKLIGKLNWASQIYAGIKV

SEQ ID NO: 225 - protein, 25 aa, synthetic construct
KKHQKEPPFLWMGYELHPDKWTVQP

SEQ ID NO: 226 - protein, 25 aa, synthetic construct
KKHQKDPPFLWMGYELHPDRWTVQP

SEQ ID NO: 227 - protein, 25 aa, synthetic construct
PPFLWMGYELHPDKWTVQPIVLPEK

SEQ ID NO: 228 - protein, 25 aa, synthetic construct
PPFLWMGYELHPDRWTVQPIELPEK

SEQ ID NO: 229 - protein, 9 aa, synthetic construct
FLWMGYELH

SEQ ID NO: 230 - protein, 9 aa, synthetic construct
ELHPDKWTV

SEQ ID NO: 231 - protein, 9 aa, synthetic construct
ELHPDRWTV

SEQ ID NO: 232 - protein, 25 aa, synthetic construct
IVLPEKDSWTVNDIQKLVGKLNWAS

SEQ ID NO: 233 - protein, 25 aa, synthetic construct
IELPEKESWTVNDIQKLIGKLNWAS

SEQ ID NO: 234 - protein, 25 aa, synthetic construct
VLPEKDSWTVNDIQKLVGKLNWASQ

SEQ ID NO: 235 - protein, 25 aa, synthetic construct
ELPEKESWTVNDIQKLIGKLNWASQ

SEQ ID NO: 236 - protein, 31 aa, synthetic construct
LPEKDSWTVNDIQKLVGKLNWASQIYPGIKV

SEQ ID NO: 237 - protein, 31 aa, synthetic construct
LPEKESWTVNDIQKLIGKLNWASQIYAGIKV

SEQ ID NO: 238 - protein, 25 aa, synthetic construct
SWTVNDIQKLVGKLNWASQIYPGIK

SEQ ID NO: 239 - protein, 25 aa, synthetic construct
SWTVNDIQKLIGKLNWASQIYAGIK

SEQ ID NO: 240 - protein, 9 aa, synthetic construct
WTVNDIQKL

SEQ ID NO: 241 - protein, 9 aa, synthetic construct
TVNDIQKLV

SEQ ID NO: 242 - protein, 9 aa, synthetic construct
TVNDIQKLI

SEQ ID NO: 243 - protein, 9 aa, synthetic construct
KLVGKLNWA

SEQ ID NO: 244 - protein, 9 aa, synthetic construct
KLIGKLNWA

SEQ ID NO: 245 - protein, 9 aa, synthetic construct
LCKLLRGTK

SEQ ID NO: 246 - protein, 9 aa, synthetic construct
LCKLLRGAK

SEQ ID NO: 247 - protein, 19 aa, synthetic construct
EAELELAENREILKEPVHG

SEQ ID NO: 248 - protein, 19 aa, synthetic construct
EAEIELAENREILREPVHG

SEQ ID NO: 249 - protein, 12 aa, synthetic construct
EPVHGVYYDPSK

SEQ ID NO: 250 - protein, 12 aa, synthetic construct
EPVHGAYYDPSK

SEQ ID NO: 251 - protein, 22 aa, synthetic construct
GQWTYQIYQEPFKNLKTGKYAR

SEQ ID NO: 252 - protein, 22 aa, synthetic construct
GQWSYQIYQEPYKNLKTGKYAK

SEQ ID NO: 253 - protein, 16 aa, synthetic construct
AHTNDVKQLTEAVQKI

SEQ ID NO: 254 - protein, 16 aa, synthetic construct
AHTNDVRQLTEAVQKV

SEQ ID NO: 255 - protein, 10 aa, synthetic construct
IVIWGKTPKF

SEQ ID NO: 256 - protein, 10 aa, synthetic construct
IVIWGKIPKF

SEQ ID NO: 257 - protein, 13 aa, synthetic construct
PKFKLPIQKETWE

SEQ ID NO: 258 - protein, 13 aa, synthetic construct
PKFRLPIQKETWD

SEQ ID NO: 259 - protein, 65 aa, synthetic construct
PKFKLPIQKETWETWWTEYWQATWIPEWEFVNTPPLVKLWYQLEKEPIVGAETFYVDGAANRETK

SEQ ID NO: 260 - protein, 65 aa, synthetic construct
PKFRLPIQKETWDTWWTDYWQATWIPEWEFTNTPPLVKLWYQLETEPIAGVETFYVDGASNRETK

SEQ ID NO: 261 - protein, 25 aa, synthetic construct
WETWWTEYWQATWIPEWEFVNTPPL

SEQ ID NO: 262 - protein, 25 aa, synthetic construct
WDTWWTDYWQATWIPEWEFTNTPPL

SEQ ID NO: 263 - protein, 31 aa, synthetic construct
EYWQATWIPEWEFVNTPPLVKLWYQLEKEPI

SEQ ID NO: 264 - protein, 31 aa, synthetic construct
DYWQATWIPEWEFTNTPPLVKLWYQLETEPI

SEQ ID NO: 265 - protein, 9 aa, synthetic construct
WQATWIPEW

SEQ ID NO: 266 - protein, 16 aa, synthetic construct
GAETFYVDGAANRETK

SEQ ID NO: 267 - protein, 16 aa, synthetic construct
GVETFYVDGASNRETK

SEQ ID NO: 268 - protein, 13 aa, synthetic construct
TDTTNQKTELQAI

SEQ ID NO: 269 - protein, 13 aa, synthetic construct
ADTTNQKTELHAI

SEQ ID NO: 270 - protein, 9 aa, synthetic construct
AIHLALQDS

SEQ ID NO: 271 - protein, 9 aa, synthetic construct
AIYLALQDS

SEQ ID NO: 272 - protein, 33 aa, synthetic construct
LALQDSGLEVNIVTDSQYALGIIQAQPDKSESE

SEQ ID NO: 273 - protein, 33 aa, synthetic construct
LALQDSGSEVNIVTDSQYAIGIIQAQPDRSESE

SEQ ID NO: 274 - protein, 25 aa, synthetic construct
QDSGLEVNIVTDSQYALGIIQAQPD

SEQ ID NO: 275 - protein, 25 aa, synthetic construct
QDSGSEVNIVTDSQYAIGIIQAQPD

SEQ ID NO: 276 - protein, 9 aa, synthetic construct
IVTDSQYAL

SEQ ID NO: 277 - protein, 9 aa, synthetic construct
IVTDSQYAI

SEQ ID NO: 278 - protein, 9 aa, synthetic construct
VSQIIEQLI

SEQ ID NO: 279 - protein, 9 aa, synthetic construct
VNQIIEQLI

SEQ ID NO: 280 - protein, 10 aa, synthetic construct
QIIEQLIKKE

SEQ ID NO: 281 - protein, 10 aa, synthetic construct
QIIEQLINKE

SEQ ID NO: 282 - protein, 26 aa, synthetic construct
KEKVYLAWVPAHKGIGGNEQVDKLVS

SEQ ID NO: 283 - protein, 26 aa, synthetic construct
KEKIYLAWVPAHKGIGGNEQIDKLVS

SEQ ID NO: 284 - protein, 16 aa, synthetic construct
GIRKVLFLDGIDKAQE

SEQ ID NO: 285 - protein, 16 aa, synthetic construct
GIRRVLFLDGIEKAQD

SEQ ID NO: 286 - protein, 9 aa, synthetic construct
HEKYHSNWR

SEQ ID NO: 287 - protein, 9 aa, synthetic construct
HEKYHNNWR

SEQ ID NO: 288 - protein, 9 aa, synthetic construct
MASDFNLPP

SEQ ID NO: 289 - protein, 9 aa, synthetic construct
MASDFNIPP

SEQ ID NO: 290 - protein, 13 aa, synthetic construct
FNLPPVVAKEIVA

SEQ ID NO: 291 - protein, 13 aa, synthetic construct
FNLPPIVAKEIVA

SEQ ID NO: 292 - protein, 87 aa, synthetic construct
FNLPPVVAKEIVASCDKCQLKGEAMHGQVDCSPGIWQLDCTHLEGKIILVAVHVASGYIEAEVIPAETGQETAYFLLKLAGRWPVKT

SEQ ID NO: 293 - protein, 87 aa, synthetic construct
FNLPPIVAKEIVACCDKCQLKGEAIHGQVDCSPGVWQLDCTHLEGKVILVAVHVASGYIEAEIIPTETGQETAYFILKLAGRWPVTT

SEQ ID NO: 294 - protein, 81 aa, synthetic construct
VAKEIVASCDKCQLKGEAMHGQVDCSPGIWQLDCTHLEGKIILVAVHVASGYIEAEVIPAETGQETAYFLLKLAGRWPVKT

SEQ ID NO: 295 - protein, 81 aa, synthetic construct
VAKEIVACCDKCQLKGEAIHGQVDCSPGVWQLDCTHLEGKVILVAVHVASGYMEAEVIPTETGQETAYFILKLAGRWPVTT

SEQ ID NO: 296 - protein, 25 aa, synthetic construct
QLKGEAMHGQVDCSPGIWQLDCTHL

SEQ ID NO: 297 - protein, 25 aa, synthetic construct
QLKGEAIHGQVDCSPGVWQLDCTHL

SEQ ID NO: 298 - protein, 9 aa, synthetic construct
GQVDCSPGI

SEQ ID NO: 299 - protein, 9 aa, synthetic construct
GQVDCSPGV

SEQ ID NO: 300 - protein, 25 aa, synthetic construct
QVDCSPGIWQLDCTHLEGKIILVAV

SEQ ID NO: 301 - protein, 25 aa, synthetic construct
QVDCSPGVWQLDCTHLEGKVILVAV

SEQ ID NO: 302 - protein, 9 aa, synthetic construct
WQLDCTHLE

SEQ ID NO: 303 - protein, 25 aa, synthetic construct
SNFTSTTVKAACWWAGIKQEFGIPY

SEQ ID NO: 304 - protein, 25 aa, synthetic construct
SNFTSTAVKAACWWAGVKQEFGIPY

SEQ ID NO: 305 - protein, 80 aa, synthetic construct
TVKAACWWAGIKQEFGIPYNPQSQGVVESMNKELKKIIGQVRDQAEHLKTAVQMAVFIHNFKRKGGIGGYSAGERIVDII

SEQ ID NO: 306 - protein, 80 aa, synthetic construct
AVKAACWWAGVKQEFGIPYHPQSQGVVESMNNELKKIIGQIRDQAEQLKTAVQMAVLIHNFKRKGGIGEYSAGERIIDII

SEQ ID NO: 307 - protein, 81 aa, synthetic construct
TVKAACWWAGIKQEFGIPYNPQSQGVVESMNKELKKIIGQVRDQAEHLKTAVQMAVFIHNFKRKGGIGGYSAGERIVDIIA

SEQ ID NO: 308 - protein, 81 aa, synthetic construct
AVKAACWWAGVKQEFGIPYHPQSQGVVESMNNELKKIIGQIRDQAEQLKTAVQMAVLIHNFKRKGGIGEYSAGERIIDIIA

SEQ ID NO: 309 - protein, 164 aa, synthetic construct
TVKAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRDPLWKGPAKLLWKGEGAVVIQDNSDIKVVPRRKAKIIRDYGKQMAGDDCVASRQDED

SEQ ID NO: 310 - protein, 164 aa, synthetic construct
AVKAACWWAGVKQEFGIPYNTQSQGVVESMNNELKKIIGQIRDQAEHLKTAVQMAVLIHNFKRKGGIGEYSAGERIIDIIATDIQTRELQKQITKLQNFRVYYRDNRDPLWKGPARLLWKGEGAVVIQDNSEIKVVPRRKVKIIRDYGKRMAGDDCVAGRQDED

SEQ ID NO: 311 - protein, 9 aa, synthetic construct
KAACWWAGI

SEQ ID NO: 312 - protein, 9 aa, synthetic construct
KAACWWAGV

SEQ ID NO: 313 - protein, 9 aa, synthetic construct
DIIATDIQT

SEQ ID NO: 314 - protein, 9 aa, synthetic construct
DIIASDIQT

SEQ ID NO: 315 - protein, 9 aa, synthetic construct
DIQTKELQK

SEQ ID NO: 316 - protein, 9 aa, synthetic construct
DIQTRELQK

SEQ ID NO: 317 - protein, 9 aa, synthetic construct
QTKELQKQI

SEQ ID NO: 318 - protein, 9 aa, synthetic construct
QTRELQKQI

SEQ ID NO: 319 - protein, 73 aa, synthetic construct
AITKIQNFRVYYRDSRDPLWKGPAKLLWKGEGAVVIQDNSDIKVVPRRKAKIIRDYGKQMAGDDCVASRQDED

SEQ ID NO: 320 - protein, 73 aa, synthetic construct
AITKLQNFRVYYRDNRDPLWKGPARLLWKGEGAVVIQDNSEIKVVPRRKVKIIRDYGKRMAGDDCVAGRQDED

SEQ ID NO: 321 - protein, 72 aa, synthetic construct
ITKIQNFRVYYRDSRDPLWKGPAKLLWKGEGAVVIQDNSDIKVVPRRKAKIIRDYGKQMAGDDCVASRQDED

SEQ ID NO: 322 - protein, 72 aa, synthetic construct
ITKLQNFRVYYRDNRDPLWKGPARLLWKGEGAVVIQDNSEIKVVPRRKVKIIRDYGKRMAGDDCVAGRQDED

SEQ ID NO: 323 - protein, 25 aa, synthetic construct
VYYRDSRDPLWKGPAKLLWKGEGAV

SEQ ID NO: 324 - protein, 25 aa, synthetic construct
VYYRDNRDPLWKGPARLLWKGEGAV

SEQ ID NO: 325 - protein, 25 aa, synthetic construct
DPLWKGPAKLLWKGEGAVVIQDNSD

SEQ ID NO: 326 - protein, 25 aa, synthetic construct
DPLWKGPARLLWKGEGAVVIQDNSE

SEQ ID NO: 327 - protein, 9 aa, synthetic construct
PLWKGPAKL

SEQ ID NO: 328 - protein, 9 aa, synthetic construct
PLWKGPARL

SEQ ID NO: 329 - protein, 25 aa, synthetic construct
PLWKGPAKLLWKGEGAVVIQDNSDI

SEQ ID NO: 330 - protein, 25 aa, synthetic construct
PLWKGPARLLWKGEGAVVIQDNSEI

SEQ ID NO: 331 - protein, 9 aa, synthetic construct
KLLWKGEGA

SEQ ID NO: 332 - protein, 9 aa, synthetic construct
RLLWKGEGA

SEQ ID NO: 333 - protein, 9 aa, synthetic construct
LLWKGEGAV

SEQ ID NO: 334 - protein, 24 aa, synthetic construct
AKIIRDYGKQMAGDDCVASRQDED

SEQ ID NO: 335 - protein, 24 aa, synthetic construct
VKIIRDYGKRMAGDDCVAGRQDED

SEQ ID NO: 336 - protein, 9 aa, synthetic construct
KQMAGDDCV

SEQ ID NO: 337 - protein, 9 aa, synthetic construct
KRMAGDDCV

SEQ ID NO: 338 - protein, 25 aa, synthetic construct
YLRDQQLLGLWGCSGKLICPTAVPW

SEQ ID NO: 339 - protein, 26 aa, synthetic construct
PVGEIYKRWIILGLNKIVRMYSPTSI

SEQ ID NO: 340 - protein, 26 aa, synthetic construct
PVGDIYKRWIIMGLNKIVRMYSPVSI

SEQ ID NO: 341 - protein, 26 aa, synthetic construct
GPKEPFRDYVDRFYKTLRAEQASQEV

SEQ ID NO: 342 - protein, 26 aa, synthetic construct
GPKEPFRDYVDRFYRTLRAEQASQDV

SEQ ID NO: 343 - protein, 25 aa, synthetic construct
FRKQNPDIVIYQYVDDLYVGSDLEI

SEQ ID NO: 344 - protein, 25 aa, synthetic construct
KQNPDIVIYQYVDDLYVGSDLEIEQ

SEQ ID NO: 345 - protein, 771 aa, synthetic construct
LKHIVWASRELERFAVNPGLLETAAAVSQNYPIVQNAAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQAAALPGRWKPKMIGGIGGFIKVRQYDQAAAGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSAAAWGFTTPDKKHQKEPPFLWMGYELHPDKWTVQPIAAAVAKEIVASCDKCQLKGEAMHGQVDCSPGIWQLDCTHLEGKIILVAVHVASGYIEAEVIPAETGQETAYFLLKLAGRWPVKTAAATVKAACWWAGIKQEFGIPYNPQSQGVVESMNKELKKIIGQVRDQAEHLKTAVQMAVFIHNFKRKGGIGGYSAGERIVDIIAAAAITKIQNFRVYYRDSRDPLWKGPAKLLWKGEGAVVIQDNSDIKVVPRRKAKIIRDYGKQMAGDDCVASRQDEDAAAEEVGFPVKPQVPLRPMTFKGALDLSHFLREKGGLEG

SEQ ID NO: 346 - protein, 771 aa, synthetic construct
LKHLVWASRELERFALNPGLLETAAAVSQNFPIVQNAAALSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQAAALPGKWKPKMIGGIGGFIKVKQYDQAAAGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSAAAWGLTTPDKKHQKDPPFLWMGYELHPDRWTVQPIAAAVAKEIVACCDKCQLKGEAIHGQVDCSPGVWQLDCTHLEGKVILVAVHVASGYMEAEVIPTETGQETAYFILKLAGRWPVTTAAAAVKAACWWAGVKQEFGIPYHPQSQGVVESMNNELKKIIGQIRDQAEQLKTAVQMAVLIHNFKRKGGIGEYSAGERIIDIIAAAAITKLQNFRVYYRDNRDPLWKGPARLLWKGEGAVVIQDNSEIKVVPRRKVKIIRDYGKRMAGDDCVAGRQDEDAAAEEVGFPVRPQVPLRPMTYKGALDLSHFLKEKGGLEG

SEQ ID NO: 347 - protein, 800 aa, synthetic construct
LKHIVWASRELERFAVNPGLLETVSQNYPIVQNISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQRAKRAPVKQTLNFDLLKLAGDVESNPGPLPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSWGFTTPDKKHQKEPPFLWMGYELHPDKWTVQPIVAKEIVASCDKCQLKGEAMHGQVDCSPGIWQLDCTHLEGKIILVAVHVASGYIEAEVIPAETGQETAYFLLKLAGRWPVKTTVKAACWWAGIKQEFGIPYNPQSQGVVESMNKELKKIIGQVRDQAEHLKTAVQMAVFIHNFKRKGGIGGYSAGERIVDIIAITKIQNFRVYYRDSRDPLWKGPAKLLWKGEGAVVIQDNSDIKVVPRRKAKIIRDYGKQMAGDDCVASRQDEDRAKRAPVKQTLNFDLLKLAGDVESNPGPEEVGFPVKPQVPLRPMTFKGALDLSHFLREKGGLEG

SEQ ID NO: 348 - protein, 800 aa, synthetic construct
LKHLVWASRELERFALNPGLLETVSQNFPIVQNLSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQRAKRAPVKQTLNFDLLKLAGDVESNPGPLPGKWKPKMIGGIGGFIKVKQYDQGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSWGLTTPDKKHQKDPPFLWMGYELHPDRWTVQPIVAKEIVACCDKCQLKGEAIHGQVDCSPGVWQLDCTHLEGKVILVAVHVASGYMEAEVIPTETGQETAYFILKLAGRWPVTTAVKAACWWAGVKQEFGIPYHPQSQGVVESMNNELKKIIGQIRDQAEQLKTAVQMAVLIHNFKRKGGIGEYSAGERIIDIIAITKLQNFRVYYRDNRDPLWKGPARLLWKGEGAVVIQDNSEIKVVPRRKVKIIRDYGKRMAGDDCVAGRQDEDRAKRAPVKQTLNFDLLKLAGDVESNPGPEEVGFPVRPQVPLRPMTYKGALDLSHFLKEKGGLEG

SEQ ID NO: 349 - protein, 744 aa, synthetic construct
LKHIVWASRELERFAVNPGLLETVSQNYPIVQNISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQLPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSWGFTTPDKKHQKEPPFLWMGYELHPDKWTVQPIVAKEIVASCDKCQLKGEAMHGQVDCSPGIWQLDCTHLEGKIILVAVHVASGYIEAEVIPAETGQETAYFLLKLAGRWPVKTTVKAACWWAGIKQEFGIPYNPQSQGVVESMNKELKKIIGQVRDQAEHLKTAVQMAVFIHNFKRKGGIGGYSAGERIVDIIAITKIQNFRVYYRDSRDPLWKGPAKLLWKGEGAVVIQDNSDIKVVPRRKAKIIRDYGKQMAGDDCVASRQDEDEEVGFPVKPQVPLRPMTFKGALDLSHFLREKGGLEG

SEQ ID NO: 350 - protein, 744 aa, synthetic construct
LKHLVWASRELERFALNPGLLETVSQNFPIVQNLSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQLPGKWKPKMIGGIGGFIKVKQYDQGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSWGLTTPDKKHQKDPPFLWMGYELHPDRWTVQPIVAKEIVACCDKCQLKGEAIHGQVDCSPGVWQLDCTHLEGKVILVAVHVASGYMEAEVIPTETGQETAYFILKLAGRWPVTTAVKAACWWAGVKQEFGIPYHPQSQGVVESMNNELKKIIGQIRDQAEQLKTAVQMAVLIHNFKRKGGIGEYSAGERIIDIIAITKLQNFRVYYRDNRDPLWKGPARLLWKGEGAVVIQDNSEIKVVPRRKVKIIRDYGKRMAGDDCVAGRQDEDEEVGFPVRPQVPLRPMTYKGALDLSHFLKEKGGLEG

SEQ ID NO: 351 - protein, 346 aa, synthetic construct
MGARASVLSGGELDRWEKIRLRPGGKKKYRLKHIVWASRELERFAVNPGLLETAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQEEVGFPVKPQVPLRPMTFKGALDLSHFLREKGGLEGTQGFFPDWQNYTPEPGIRFPLTFGWCFKLVPL

SEQ ID NO: 352 - protein, 344 aa, synthetic construct
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQMGARASILSGGKLDKWEKIRLRPGGRKKYKLKHIVWASRELERFAVNPGLLETEEVGFPVRPQVPLRPMTYKGALDLSHFLKEKGGLEGTQGYFPDWQNYTPGPGTRYPLTFGWCFKLVPV

SEQ ID NO: 353 - protein, 386 aa, synthetic construct
MWLQSLLLLGTVACSISVMGARASVLSGGELDRWEKIRLRPGGKKKYRLKHIVWASRELERFAVNPGLLETLKHIVWASRELERFAVNPGLLETAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQEEVGFPVKPQVPLRPMTFKGALDLSHFLREKGGLEGTQGFFPDQNYTPEPGIRFPLTFGWCFKLVPL

SEQ ID NO: 354 - protein, 391 aa, synthetic construct
MDAMKRGLCCVLLLCGAVFVSARMGARASVLSGGELDRWEKIRLRPGGKKKYRLKHIVWASRELERFAVNPGLLETLKHIVWASRELERFAVNPGLLETAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQEEVGFPVKPQVPLRPMTFKGALDLSHFLREKGGLEGTQGFFPDQNYTPEPGIRFPLTFGWCFKLVPL

SEQ ID NO: 355 - protein, 475 aa, synthetic construct
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAMGARASVLSGGELDRWEKIRLRPGGKKKYRLKHIVWASRELERFAVNPGLLETLKHIVWASRELERFAVNPGLLETAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQEEVGFPVKPQVPLRPMTFKGALDLSHFLREKGGLEGTQGFFPDQNYTPEPGIRFPLTFGWCFKLVPL

SEQ ID NO: 356 - protein, 399 aa, synthetic construct
MRKAAVSHWQQQSYLDSGIHSGATTTAPSLSMGARASVLSGGELDRWEKIRLRPGGKKKYRLKHIVWASRELERFAVNPGLLETLKHIVWASRELERFAVNPGLLETAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQEEVGFPVKPQVPLRPMTFKGALDLSHFLREKGGLEGTQGFFPDQNYTPEPGIRFPLTFGWCFKLVPL

SEQ ID NO: 357 - protein, 854 aa, synthetic construct
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTTVKAACWWAGIKQEFGIPYNPQSQGVVESMNKELKKIIGQVRDQAEHLKTAVQMAVFIHNFKRKGGIGGYSAGERIVDIINVSTVQCTHGIRPVVSTQLLLNGSLAEEKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTVAKEIVASCDKCQLKGEAMHGQVDCSPGIWQLDCTHLEGKIILVAVHVASGYIEAEVIPAETGQETAYFLLKLAGRWPVKTLWVTVYYGVPVWKEAAFPQITLWQRPLVTIKIGGQLKEALLDTGADDTVLEEMNLPGRWKPKMIGGIGGFIKVRQYDQAAAAHNVWATHACVPTDPNPQEAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRMRDNWRSELYKYKVV

SEQ ID NO: 358 - protein, 849 aa, synthetic construct
AVKAACWWAGVKQEFGIPYHPQSQGVVESMNNELKKIIGQIRDQAEQLKTAVQMAVLIHNFKRKGGIGEYSAGERIIDIIRRRVVQREKRAIGLGAVFLGFLGTAGSTMGAASMTLTVQARLLLSGIVQQQSNLLRAIEAQQHMLQLTVWGIKQLQARILAVERYLRDQQLLGIWGCSGRLICTTVAKEIVACCDKCQLKGEAIHGQVDCSPGVWQLDCTHLEGKVILVAVHVASGYMEAEVIPTETGQETAYFILKLAGRWPVTTNISTVQCTHGIKPVVSTQLLLNGSLAEKWGLTTPDKKHQKDPPFLWMGYELHPDRWTVQPIELPEKESWTVNDIQKLIGKLNWASQIYAGIKVIVIYQYVDDLYVGSDLEIEQHRPKFRLPIQKETWDTWWTDYWQATWIPEWEFTNTPPLVKLWYQLETEPIAGVETFYVDGASNRETKLPQITLWQRPIVTIKIGGQIKEALLDTGADDTVLEDMNLPGKWKPKMIGGIGGFIKVKQYDQAALWVTIYYGVPVWKDVHNIWATHACVPTDPSPQEAITKLQNFRVYYRDNRDPLWKGPARLLWKGEGAVVIQDNSEIKVVPRRKVKIIRDYGKRMAGDDCVAGRQDEDGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTKEKIYLAWVPAHKGIGGNEQIDKLVSMKDNWRSELYRYKVV

SEQ ID NO: 359 - protein, 486 aa, synthetic construct
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTTVKAACWWAGIKQEFGIPYNPQSQGVVESMNKELKKIIGQVRDQAEHLKTAVQMAVFIHNFKRKGGIGGYSAGERIVDIINVSTVQCTHGIRPVVSTQLLLNGSLAEEKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTVAKEIVASCDKCQLKGEAMHGQVDCSPGIWQLDCTHLEGKIILVAVHVASGYIEAEVIPAETGQETAYFLLKLAGRWPVKT

SEQ ID NO: 360 - protein, 368 aa, synthetic construct
LWVTVYYGVPVWKEAAFPQITLWQRPLVTIKIGGQLKEALLDTGADDTVLEEMNLPGRWKPKMIGGIGGFIKVRQYDQAAAAHNVWATHACVPTDPNPQEAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRMRDNWRSELYKYKVV

SEQ ID NO: 361 - protein, 510 aa, synthetic construct
AVKAACWWAGVKQEFGIPYHPQSQGVVESMNNELKKIIGQIRDQAEQLKTAVQMAVLIHNFKRKGGIGEYSAGERIIDIIRRRVVQREKRAIGLGAVFLGFLGTAGSTMGAASMTLTVQARLLLSGIVQQQSNLLRAIEAQQHMLQLTVWGIKQLQARILAVERYLRDQQLLGIWGCSGRLICTTVAKEIVACCDKCQLKGEAIHGQVDCSPGVWQLDCTHLEGKVILVAVHVASGYMEAEVIPTETGQETAYFILKLAGRWPVTTNISTVQCTHGIKPVVSTQLLLNGSLAEKWGLTTPDKKHQKDPPFLWMGYELHPDRWTVQPIELPEKESWTVNDIQKLIGKLNWASQIYAGIKVIVIYQYVDDLYVGSDLEIEQHRPKFRLPIQKETWDTWWTDYWQATWIPEWEFTNTPPLVKLWYQLETEPIAGVETFYVDGASNRETKLPQITLWQRPIVTIKIGGQIKEALLDTGADDTVLEDMNLPGKWKPKMIGGIGGFIKVKQYDQAA

SEQ ID NO: 362 - protein, 339 aa, synthetic construct
LWVTIYYGVPVWKDVHNIWATHACVPTDPSPQEAITKLQNFRVYYRDNRDPLWKGPARLLWKGEGAVVIQDNSEIKVVPRRKVKIIRDYGKRMAGDDCVAGRQDEDGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTKEKIYLAWVPAHKGIGGNEQIDKLVSMKDNWRSELYRYKVV

SEQ ID NO: 363 - protein, 872 aa, synthetic construct
MWLQSLLLLGTVACSISVGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTTVKAACWWAGIKQEFGIPYNPQSQGVVESMNKELKKIIGQVRDQAEHLKTAVQMAVFIHNFKRKGGIGGYSAGERIVDIINVSTVQCTHGIRPVVSTQLLLNGSLAEEKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTVAKEIVASCDKCQLKGEAMHGQVDCSPGIWQLDCTHLEGKIILVAVHVASGYIEAEVIPAETGQETAYFLLKLAGRWPVKTLWVTVYYGVPVWKEAAFPQITLWQRPLVTIKIGGQLKEALLDTGADDTVLEEMNLPGRWKPKMIGGIGGFIKVRQYDQAAAAHNVWATHACVPTDPNPQEAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRMRDNWRSELYKYKVV

SEQ ID NO: 364 - protein, 877 aa, synthetic construct
MDAMKRGLCCVLLLCGAVFVSARGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTTVKAACWWAGIKQEFGIPYNPQSQGVVESMNKELKKIIGQVRDQAEHLKTAVQMAVFIHNFKRKGGIGGYSAGERIVDIINVSTVQCTHGIRPVVSTQLLLNGSLAEEKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTVAKEIVASCDKCQLKGEAMHGQVDCSPGIWQLDCTHLEGKIILVAVHVASGYIEAEVIPAETGQETAYFLLKLAGRWPVKTLWVTVYYGVPVWKEAAFPQITLWQRPLVTIKIGGQLKEALLDTGADDTVLEEMNLPGRWKPKMIGGIGGFIKVRQYDQAAAAHNVWATHACVPTDPNPQEAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRMRDNWRSELYKYKVV

SEQ ID NO: 365 - protein, 961 aa, synthetic construct
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTTVKAACWWAGIKQEFGIPYNPQSQGVVESMNKELKKIIGQVRDQAEHLKTAVQMAVFIHNFKRKGGIGGYSAGERIVDIINVSTVQCTHGIRPVVSTQLLLNGSLAEEKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTVAKEIVASCDKCQLKGEAMHGQVDCSPGIWQLDCTHLEGKIILVAVHVASGYIEAEVIPAETGQETAYFLLKLAGRWPVKTLWVTVYYGVPVWKEAAFPQITLWQRPLVTIKIGGQLKEALLDTGADDTVLEEMNLPGRWKPKMIGGIGGFIKVRQYDQAAAAHNVWATHACVPTDPNPQEAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRMRDNWRSELYKYKVV

SEQ ID NO: 366 - protein, 885 aa, synthetic construct
MRKAAVSHWQQQSYLDSGIHSGATTTAPSLSGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTTVKAACWWAGIKQEFGIPYNPQSQGVVESMNKELKKIIGQVRDQAEHLKTAVQMAVFIHNFKRKGGIGGYSAGERIVDIINVSTVQCTHGIRPVVSTQLLLNGSLAEEKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTVAKEIVASCDKCQLKGEAMHGQVDCSPGIWQLDCTHLEGKIILVAVHVASGYIEAEVIPAETGQETAYFLLKLAGRWPVKTLWVTVYYGVPVWKEAAFPQITLWQRPLVTIKIGGQLKEALLDTGADDTVLEEMNLPGRWKPKMIGGIGGFIKVRQYDQAAAAHNVWATHACVPTDPNPQEAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRMRDNWRSELYKYKVV

SEQ ID NO: 367 - protein, 363 aa, synthetic construct
YQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLRAEQASQEVDLNTMLNTVKLTPLCVTLYQYMDDLYVVIYQYMDDL

SEQ ID NO: 368 - protein, 381 aa, synthetic construct
MWLQSLLLLGTVACSISVYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLRAEQASQEVDLNTMLNTVKLTPLCVTLYQYMDDLYVVIYQYMDDL

SEQ ID NO: 369 - protein, 386 aa, synthetic construct
MDAMKRGLCCVLLLCGAVFVSARYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLRAEQASQEVDLNTMLNTVKLTPLCVTLYQYMDDLYVVIYQYMDDL

SEQ ID NO: 370 - protein, 470 aa, synthetic construct
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLRAEQASQEVDLNTMLNTVKLTPLCVTLYQYMDDLYVVIYQYMDDL

SEQ ID NO: 371 - protein, 394 aa, synthetic construct
MRKAAVSHWQQQSYLDSGIHSGATTTAPSLSYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLRAEQASQEVDLNTMLNTVKLTPLCVTLYQYMDDLYVVIYQYMDDL

SEQ ID NO: 372 - protein, 928 aa, synthetic construct
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTLLPDARDPAFKAANGSLRALQATVGNSYKCNAEEHVRVTKAFSVNIFKVWVQAFKVEGGQFGSVEECLLDENSLEDIRTLNAWVKVREKRDLNTMLNTVREKRWIILGLNKIREKRYVDRFYKTLREKRATLEEMMTAREKREMMTACQGVREKRTLNFPISPIREKRYQYNVLPQGREKRVIYQYMDDLREKRYQYMDDLYVREKRYMDDLYVGSREKRFLWMGYELHREKRELHPDKWTVREKRWTVNDIQKLREKRTVNDIQKLVREKRKLVGKLNWAREKRWQATWIPEWREKRIVTDSQYALREKRGQVDCSPGIREKRWQLDCTHLEREKRKAACWWAGIREKRPLWKGPAKLREKRKLLWKGEGAREKRLLWKGEGAVREKRKQMAGDDCVREKRVTVYYGVPVREKRNVWATHACVREKRKLTPLCVTLREKRSTVQCTHGIREKRGIWGCSGKLREKRLTFGWCFKLREKRASRELERFAVNPGLLREKRWIILGLNKIVRMYSPTSIREKRYVDRFYKTLRAEQASQEVREKRRQANFLGKIWPSHKGRREKRYKAAVDLSHFLREKGGLEGGSEFTLIPIAVGGALAGLVIVLIAYLVGRKRSHAGYQTI

SEQ ID NO: 373 - protein, 847 aa, synthetic construct
ASRELERFAVNPGLLQLKGEAMHGQVDCSPGIWQLDCTHLCSATEKLWVTVYYGVPVWKEATTTLPVGEIYKRWIILGLNKIVRMYSPTSINNETPGIRYQYNVLPQGWKGSPAIFAAPLWKGPAKLLWKGEGAVVIQDNSDIAAIVLPEKDSWTVNDIQKLVGKLNWASVYYRDSRDPLWKGPAKLLWKGEGAVSNFTSTTVKAACWWAGIKQEFGIPYVLPEKDSWTVNDIQKLVGKLNWASQMVHQAISPRTLNAWVKVVEEKAFSPAAWETWWTEYWQATWIPEWEFVNTPPLQDSGLEVNIVTDSQYALGIIQAQPDSWTVNDIQKLVGKLNWASQIYPGIKNPDIVIYQYMDDLYVGSDLEIGQHRAADPLWKGPAKLLWKGEGAVVIQDNSDRQANFLGKIWPSHKGRAAPPFLWMGYELHPDKWTVQPIVLPEKFRKQNPDIVIYQYMDDLYVGSDLEINLLTQIGCTLNFPISPIETVPVKLKGPKEPFRDYVDRFYKTLRAEQASQEVYLKDQQLLGIWGCSGKLICTTAVPWAAAKKHQKEPPFLWMGYELHPDKWTVQPGTGPCTNVSTVQCTHGIRPVVSTQLKQNPDIVIYQYMDDLYVGSDLEIGQLGPAATLEEMMTACQGVGGPGHKARDQSLKPCVKLTPLCVTLNCTDLRNTGPGIRYPLLTFGWCFKLPVEPEKVEAKIIRDYGKQMAGDDCVASRQDEDILKALGPAATLEEMMTACQGVGGPGYKAAVDLSHFLREKGGLEGAAYAAKAYDTEVHNVWATHACVPTDPNPQEAAALSEGATPQDLNTMLNTVGGHQAAMQQVDCSPGIWQLDCTHLEGKIILVAV

SEQ ID NO: 374 - protein, 865 aa, synthetic construct
MWLQSLLLLGTVACSISVASRELERFAVNPGLLQLKGEAMHGQVDCSPGIWQLDCTHLCSATEKLWVTVYYGVPVWKEATTTLPVGEIYKRWIILGLNKIVRMYSPTSINNETPGIRYQYNVLPQGWKGSPAIFAAPLWKGPAKLLWKGEGAVVIQDNSDIAAIVLPEKDSWTVNDIQKLVGKLNWASVYYRDSRDPLWKGPAKLLWKGEGAVSNFTSTTVKAACWWAGIKQEFGIPYVLPEKDSWTVNDIQKLVGKLNWASQMVHQAISPRTLNAWVKVVEEKAFSPAAWETWWTEYWQATWIPEWEFVNTPPLQDSGLEVNIVTDSQYALGIIQAQPDSWTVNDIQKLVGKLNWASQIYPGIKNPDIVIYQYMDDLYVGSDLEIGQHRAADPLWKGPAKLLWKGEGAVVIQDNSDRQANFLGKIWPSHKGRAAPPFLWMGYELHPDKWTVQPIVLPEKFRKQNPDIVIYQYMDDLYVGSDLEINLLTQIGCTLNFPISPIETVPVKLKGPKEPFRDYVDRFYKTLRAEQASQEVYLKDQQLLGIWGCSGKLICTTAVPWAAAKKHQKEPPFLWMGYELHPDKWTVQPGTGPCTNVSTVQCTHGIRPVVSTQLKQNPDIVIYQYMDDLYVGSDLEIGQLGPAATLEEMMTACQGVGGPGHKARDQSLKPCVKLTPLCVTLNCTDLRNTGPGIRYPLLTFGWCFKLPVEPEKVEAKIIRDYGKQMAGDDCVASRQDEDILKALGPAATLEEMMTACQGVGGPGYKAAVDLSHFLREKGGLEGAAYAAKAYDTEVHNVWATHACVPTDPNPQEAAALSEGATPQDLNTMLNTVGGHQAAMQQVDCSPGIWQLDCTHLEGKIILVAV

SEQ ID NO: 375 - protein, 870 aa, synthetic construct
MDAMKRGLCCVLLLCGAVFVSARASRELERFAVNPGLLQLKGEAMHGQVDCSPGIWQLDCTHLCSATEKLWVTVYYGVPVWKEATTTLPVGEIYKRWIILGLNKIVRMYSPTSINNETPGIRYQYNVLPQGWKGSPAIFAAPLWKGPAKLLWKGEGAVVIQDNSDIAAIVLPEKDSWTVNDIQKLVGKLNWASVYYRDSRDPLWKGPAKLLWKGEGAVSNFTSTTVKAACWWAGIKQEFGIPYVLPEKDSWTVNDIQKLVGKLNWASQMVHQAISPRTLNAWVKVVEEKAFSPAAWETWWTEYWQATWIPEWEFVNTPPLQDSGLEVNIVTDSQYALGIIQAQPDSWTVNDIQKLVGKLNWASQIYPGIKNPDIVIYQYMDDLYVGSDLEIGQHRAADPLWKGPAKLLWKGEGAVVIQDNSDRQANFLGKIWPSHKGRAAPPFLWMGYELHPDKWTVQPIVLPEKFRKQNPDIVIYQYMDDLYVGSDLEINLLTQIGCTLNFPISPIETVPVKLKGPKEPFRDYVDRFYKTLRAEQASQEVYLKDQQLLGIWGCSGKLICTTAVPWAAAKKHQKEPPFLWMGYELHPDKWTVQPGTGPCTNVSTVQCTHGIRPVVSTQLKQNPDIVIYQYMDDLYVGSDLEIGQLGPAATLEEMMTACQGVGGPGHKARDQSLKPCVKLTPLCVTLNCTDLRNTGPGIRYPLLTFGWCFKLPVEPEKVEAKIIRDYGKQMAGDDCVASRQDEDILKALGPAATLEEMMTACQGVGGPGYKAAVDLSHFLREKGGLEGAAYAAKAYDTEVHNVWATHACVPTDPNPQEAAALSEGATPQDLNTMLNTVGGHQAAMQQVDCSPGIWQLDCTHLEGKIILVAV

SEQ ID NO: 376 - protein, 954 aa, synthetic construct
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAASRELERFAVNPGLLQLKGEAMHGQVDCSPGIWQLDCTHLCSATEKLWVTVYYGVPVWKEATTTLPVGEIYKRWIILGLNKIVRMYSPTSINNETPGIRYQYNVLPQGWKGSPAIFAAPLWKGPAKLLWKGEGAVVIQDNSDIAAIVLPEKDSWTVNDIQKLVGKLNWASVYYRDSRDPLWKGPAKLLWKGEGAVSNFTSTTVKAACWWAGIKQEFGIPYVLPEKDSWTVNDIQKLVGKLNWASQMVHQAISPRTLNAWVKVVEEKAFSPAAWETWWTEYWQATWIPEWEFVNTPPLQDSGLEVNIVTDSQYALGIIQAQPDSWTVNDIQKLVGKLNWASQIYPGIKNPDIVIYQYMDDLYVGSDLEIGQHRAADPLWKGPAKLLWKGEGAVVIQDNSDRQANFLGKIWPSHKGRAAPPFLWMGYELHPDKWTVQPIVLPEKFRKQNPDIVIYQYMDDLYVGSDLEINLLTQIGCTLNFPISPIETVPVKLKGPKEPFRDYVDRFYKTLRAEQASQEVYLKDQQLLGIWGCSGKLICTTAVPWAAAKKHQKEPPFLWMGYELHPDKWTVQPGTGPCTNVSTVQCTHGIRPVVSTQLKQNPDIVIYQYMDDLYVGSDLEIGQLGPAATLEEMMTACQGVGGPGHKARDQSLKPCVKLTPLCVTLNCTDLRNTGPGIRYPLLTFGWCFKLPVEPEKVEAKIIRDYGKQMAGDDCVASRQDEDILKALGPAATLEEMMTACQGVGGPGYKAAVDLSHFLREKGGLEGAAYAAKAYDTEVHNVWATHACVPTDPNPQEAAALSEGATPQDLNTMLNTVGGHQAAMQQVDCSPGIWQLDCTHLEGKIILVAV

SEQ ID NO: 377 - protein, 878 aa, synthetic construct
MRKAAVSHWQQQSYLDSGIHSGATTTAPSLSASRELERFAVNPGLLQLKGEAMHGQVDCSPGIWQLDCTHLCSATEKLWVTVYYGVPVWKEATTTLPVGEIYKRWIILGLNKIVRMYSPTSINNETPGIRYQYNVLPQGWKGSPAIFAAPLWKGPAKLLWKGEGAVVIQDNSDIAAIVLPEKDSWTVNDIQKLVGKLNWASVYYRDSRDPLWKGPAKLLWKGEGAVSNFTSTTVKAACWWAGIKQEFGIPYVLPEKDSWTVNDIQKLVGKLNWASQMVHQAISPRTLNAWVKVVEEKAFSPAAWETWWTEYWQATWIPEWEFVNTPPLQDSGLEVNIVTDSQYALGIIQAQPDSWTVNDIQKLVGKLNWASQIYPGIKNPDIVIYQYMDDLYVGSDLEIGQHRAADPLWKGPAKLLWKGEGAVVIQDNSDRQANFLGKIWPSHKGRAAPPFLWMGYELHPDKWTVQPIVLPEKFRKQNPDIVIYQYMDDLYVGSDLEINLLTQIGCTLNFPISPIETVPVKLKGPKEPFRDYVDRFYKTLRAEQASQEVYLKDQQLLGIWGCSGKLICTTAVPWAAAKKHQKEPPFLWMGYELHPDKWTVQPGTGPCTNVSTVQCTHGIRPVVSTQLKQNPDIVIYQYMDDLYVGSDLEIGQLGPAATLEEMMTACQGVGGPGHKARDQSLKPCVKLTPLCVTLNCTDLRNTGPGIRYPLLTFGWCFKLPVEPEKVEAKIIRDYGKQMAGDDCVASRQDEDILKALGPAATLEEMMTACQGVGGPGYKAAVDLSHFLREKGGLEGAAYAAKAYDTEVHNVWATHACVPTDPNPQEAAALSEGATPQDLNTMLNTVGGHQAAMQQVDCSPGIWQLDCTHLEGKIILVAV

SEQ ID NO: 378-380 - skipped (3 entries)

SEQ ID NO: 381 - protein, 4 aa, synthetic construct
RAKR

SEQ ID NO: 382 - protein, 4 aa, synthetic construct
REKR

SEQ ID NO: 383 - protein, 4 aa, synthetic construct
RRKR

SEQ ID NO: 384 - protein, 19 aa, synthetic construct
ATNFSLLKQAGDVEENPGP

SEQ ID NO: 385 - protein, 24 aa, synthetic construct
APVKQTLNFDLLKLAGDVESNPGP

SEQ ID NO: 386 - protein, 28 aa, synthetic construct
RAKRAPVKQTLNFDLLKLAGDVESNPGP

SEQ ID NO: 387 - protein, 20 aa, synthetic construct
QCTNYALLKLAGDVESNPGP

SEQ ID NO: 388 - protein, 18 aa, synthetic construct
EGRGSLLTCGDVEENPGP

SEQ ID NO: 389 - protein, 7 aa, synthetic construct
VHAGPIA

SEQ ID NO: 390 - protein, 7 aa, synthetic construct
VHAGPVA

SEQ ID NO: 391 - protein, 5 aa, synthetic construct
GALDI

SEQ ID NO: 392 - protein, 5 aa, synthetic construct
GALDL

SEQ ID NO: 393 - protein, 18 aa, synthetic construct
MWLQSLLLLGTVACSISV

SEQ ID NO: 394 - protein, 23 aa, synthetic construct
MDAMKRGLCCVLLLCGAVFVSAR

SEQ ID NO: 395 - protein, 16 aa, synthetic construct
MHRRRSRSCREDQKPV

SEQ ID NO: 396 - protein, 17 aa, synthetic construct
KWVTFISLLFLFSSAYS

SEQ ID NO: 397 - protein, 31 aa, synthetic construct
MRKAAVSHWQQQSYLDSGIHSGATTTAPSLS

SEQ ID NO: 398 - protein, 107 aa, synthetic construct
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGA

SEQ ID NO: 399 - protein, 384 aa, synthetic construct
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTLLPDARDPAFKAANGSLRALQATVGNSYKCNAEEHVRVTKAFSVNIFKVWVQAFKVEGGQFGSVEECLLDENSLEDI

SEQ ID NO: 400 - protein, 76 aa, synthetic construct [sequence omitted]

SEQ ID NO: 401 - protein, 17 aa, synthetic construct
MLLSVPLLLGLLGLAVA

SEQ ID NO: 402 - protein, 16 aa, synthetic construct
MKCLLYLAFLFIGVNC

SEQ ID NO: 403 - protein, 856 aa, Human immunodeficiency virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 404 - protein, 500 aa, Human immunodeficiency virus 1
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 405 - protein, 206 aa, Human immunodeficiency virus 1
MGGKWSKSSVIGWPTVRERMRRAEPAADRVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKIEEANKGENTSLLHPVSLHGMDDPEREVLEWRFDSRLAFHHVARELHPEYFKNC

SEQ ID NO: 406 - protein, 1003 aa, Human immunodeficiency virus 1
FFREDLAFLQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 407 - protein, 670 aa, synthetic construct
TVKAACWWAGIKQEFGIPYNPQSQGVVESMNKELKKIIGQVRDQAEHLKTAVQMAVFIHNFKRKGGIGGYSAGERIVDIIAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRVAKEIVASCDKCQLKGEAMHGQVDCSPGIWQLDCTHLEGKIILVAVHVASGYIEAEVIPAETGQETAYFLLKLAGRWPVKTAAFPQITLWQRPLVTIKIGGQLKEALLDTGADDTVLEEMNLPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 408 - protein, 669 aa, synthetic construct
AVKAACWWAGVKQEFGIPYHPQSQGVVESMNNELKKIIGQIRDQAEQLKTAVQMAVLIHNFKRKGGIGEYSAGERIIDIIVAKEIVACCDKCQLKGEAIHGQVDCSPGVWQLDCTHLEGKVILVAVHVASGYMEAEVIPTETGQETAYFILKLAGRWPVTTWGLTTPDKKHQKDPPFLWMGYELHPDRWTVQPIELPEKESWTVNDIQKLIGKLNWASQIYAGIKVIVIYQYVDDLYVGSDLEIEQHRLPQITLWQRPIVTIKIGGQIKEALLDTGADDTVLEDMNLPGKWKPKMIGGIGGFIKVKQYDQPKFRLPIQKETWDTWWTDYWQATWIPEWEFTNTPPLVKLWYQLETEPIAGVETFYVDGASNRETKAAYAITKLQNFRVYYRDNRDPLWKGPARLLWKGEGAVVIQDNSEIKVVPRRKVKIIRDYGKRMAGDDCVAGRQDEDGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTKEKIYLAWVPAHKGIGGNEQIDKLVS

SEQ ID NO: 409 - protein, 648 aa, synthetic construct
TVKAACWWAGIKQEFGIPYNPQSQGVVESMNKELKKIIGQVRDQAEHLKTAVQMAVFIHNFKRKGGIGGYSAGERIVDIIVAKEIVASCDKCQLKGEAMHGQVDCSPGIWQLDCTHLEGKIILVAVHVASGYIEAEVIPAETGQETAYFLLKLAGRWPVKTAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVAAFPQITLWQRPLVTIKIGGQLKEALLDTGADDTVLEEMNLPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 410 - protein, 650 aa, synthetic construct
AVKAACWWAGVKQEFGIPYHPQSQGVVESMNNELKKIIGQIRDQAEQLKTAVQMAVLIHNFKRKGGIGEYSAGERIIDIIWGLTTPDKKHQKDPPFLWMGYELHPDRWTVQPIELPEKESWTVNDIQKLIGKLNWASQIYAGIKVAAYVAKEIVACCDKCQLKGEAIHGQVDCSPGVWQLDCTHLEGKVILVAVHVASGYMEAEVIPTETGQETAYFILKLAGRWPVTTLPQITLWQRPIVTIKIGGQIKEALLDTGADDTVLEDMNLPGKWKPKMIGGIGGFIKVKQYDQPKFRLPIQKETWDTWWTDYWQATWIPEWEFTNTPPLVKLWYQLETEPIAGVETFYVDGASNRETKAAYAITKLQNFRVYYRDNRDPLWKGPARLLWKGEGAVVIQDNSEIKVVPRRKVKIIRDYGKRMAGDDCVAGRQDEDGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTKEKIYLAWVPAHKGIGGNEQIDKLVS

SEQ ID NO: 411 - protein, 768 aa, synthetic construct
QLKGEAMHGQVDCSPGIWQLDCTHLCSATEKLWVTVYYGVPVWKEATTTLASRELERFAVNPGLLNNETPGIRYQYNVLPQGWKGSPAIFPVGEIYKRWIILGLNKIVRMYSPTSIAAPPFLWMGYELHPDKWTVQPIVLPEKWETWWTEYWQATWIPEWEFVNTPPLQDSGLEVNIVTDSQYALGIIQAQPDSWTVNDIQKLVGKLNWASQIYPGIKAADPLWKGPAKLLWKGEGAVVIQDNSDRQANFLGKIWPSHKGRPLWKGPAKLLWKGEGAVVIQDNSDIAASNFTSTTVKAACWWAGIKQEFGIPYVLPEKDSWTVNDIQKLVGKLNWASQMVHQAISPRTLNAWVKVVEEKAFSPIVLPEKDSWTVNDIQKLVGKLNWASGTGPCTNVSTVQCTHGIRPVVSTQLYLKDQQLLGIWGCSGKLICTTAVPWAAAKKHQKEPPFLWMGYELHPDKWTVQPVYYRDSRDPLWKGPAKLLWKGEGAVNLLTQIGCTLNFPISPIETVPVKLKGPKEPFRDYVDRFYKTLRAEQASQEVLGPAATLEEMMTACQGVGGPGHKARDQSLKPCVKLTPLCVTLNCTDLRNTGPGIRYPLLTFGWCFKLPVEPEKVEAKIIRDYGKQMAGDDCVASRQDEDILKALGPAATLEEMMTACQGVGGPGYKAAVDLSHFLREKGGLEGAAYAAKAYDTEVHNVWATHACVPTDPNPQEAAALSEGATPQDLNTMLNTVGGHQAAMQQVDCSPGIWQLDCTHLEGKIILVAV

SEQ ID NO: 412 - protein, 39 aa, synthetic construct
GSEFTLIPIAVGGALAGLVIVLIAYLVGRKRSHAGYQTI

SEQ ID NO: 413 - protein, 21 aa, synthetic construct
MNQTAILICCLIFLTLSGIQG

SEQ ID NO: 414 - DNA, 1104 bp, synthetic construct
  source 1..1104: mol_type = other DNA
atgggagctagagctagcgtgctgagcggaggagaactcgatcgctgggaaaagatcagactgagaccaggaggcaagaagaagtacagactgaagcacatcgtctgggcttctagagaactggaaagattcgccgtgaatccaggactgctggaaacactgaagcacattgtctgggctagcagagaactggagagatttgccgtgaatccaggactgctggaaacagcagctatctctcctagaacactgaacgcttgggtgaaagtggtggaggaaaaggcctttagcccagaagtgatccctatgtttagcgccctgtcagaaggagctacacctcaggatctgaacaccatgctgaacacagtgggaggacatcaggcagctatgcagatgctgaaggagacaattaacgaagaagccgccgagtgggatagactgcatccagtgcacgcaggacctattgctccaggacagatgagagagcctagaggaagcgatatcgcaggaacaacatctacactgcaggagcagatcggttggatgaccaataatcctcctatcccagtgggcgaaatctataagcgctggatcatcctgggactgaacaagatcgtgaggatgtacagccctaccagcatcctggatatcagacagggacctaaggagcctttcagagattacgtggacaggttctacaagacactgagagccgaacaggcttctcaggaggtgaagaattggatgaccgagacactgctggtgcagaacgctaatccagattgcaagacaattctgaaagctctgggaccagccgctacactggaagagatgatgaccgcttgtcagggagtgggaggaccaggacataaagctagagtgctggcagaagccatgtctcaggaagaagtgggattcccagtgaaacctcaggtgcctctgagacctatgacctttaagggagctctggacctgtctcacttcctgagagaaaagggaggactggaaggaacacagggatttttcccagatcagaattacacaccagagccaggaatcagattccctctgacattcggttggtgcttcaaactggtgcctctg

SEQ ID NO: 415 - DNA, 2562 bp, synthetic construct
  source 1..2562: mol_type = other DNA
ggaacagtgctggtgggacctactccagtgaatatcatcggaaggaacctgctgacacagattggttgtaccctgaacttccctatctctcctatcgagacagtgccagtgaaactgaagccaggaatggatggacctaaagtcaagcagtggcctctgacagaagagaagatcaaagccctggtggagatttgcaccgagatggagaaggagggaaagatcagcaagatcggcccagagaatccttacaacaccccagtgttcgccatcaagaagaaggatagcaccaagtggagaaagctggtggatttcagggagctgaacaagagaacccaggatttttgggaggtgcagctgggtattccacatcctgccggactgaaaaagaagaaaagcgtgacagtgctggacgtgggagacgcttatttcagcgtgcctctggataaggacttcagaaagtacaccgccttcaccatcccttctatcaacaacgagaccccaggaatcagataccagtacaacgtgctgcctcaaggttggaaaggatctccagccatctttcagagcagcatgacaacagtgaaggcagcttgttggtgggcaggaattaagcaggagttcggcatcccttacaatcctcagtctcagggagtggtggaatctatgaacaaggagctgaagaagatcatcggacaggtgagagatcaggccgaacatctgaagacagcagtgcaaatggccgtgttcatccacaacttcaagagaaagggcggcattggaggctattctgccggagagagaattgtggacatcatcaacgtgtcaacagtccagtgtacacacggaatcagaccagtcgtgtctacacaactgctgctgaacggatctctggccgaagagaagagaagagtggtgcagagagagaaaagagcagtgggaatcggagctatgtttctgggatttctgggcgcagcaggatctacaatgggagcagcttctatcacactgacagtgcaggctagacaactgctgagcggaattgtgcagcagcagaataacctgctgagagctatcgaagctcagcaacatctgctgcaactcaccgtctggggaattaagcaactgcaagctagagtgctggcagtggaaagatacctgaaggatcagcaactgctgggaatttggggttgctcaggcaagctgatttgcacaaccgtggccaaagagattgtggcttcttgcgacaagtgtcagctgaaaggagaagctatgcacggacaagtggattgttctccaggaatttggcagctggattgtacacacctggagggaaagattattctggtggcagtgcacgtggccagcggatatattgaagccgaggtgattccagcagaaacaggacaggaaacagcctattttctcctgaaactggcaggtaggtggccagtgaaaaccctctgggtgacagtgtactacggagtcccagtctggaaagaagcagctttccctcagattactctctggcagagacctctggtgacaatcaagatcggcggacagctgaaagaagctctgctggatacaggagcagacgatacagtgctggaagaaatgaacctgccaggtagatggaagcctaagatgatcggaggcatcggaggattcatcaaggtgagacagtacgaccaagcagcagcagctcataacgtctgggctacacacgcttgcgtgcctacagatcctaatcctcaggaagccatcaccaagatccagaatttcagggtgtactacagggacagcagagatcctctctggaaaggaccagctaaactgctgtggaaaggagaaggagcagtggtgatccaggataacagcgacatcaaggtggtgcctagaagaaaggccaagatcatcagggactacggaaagcaaatggcaggagacgattgcgtggcttctagacaggacgaggatcccaagttcaagctgcctattcagaaggagacttgggagacttggtggacagagtattggcaagcaacttggatccccgagtgggaatttgtgaatacccctcctctggtcaagctctggtatcagctggaaaaggagcctatcgtgggagccgaaacattttacgtggacggagcagctaatagagagacaaaagccgccaaggagaaagtgtatctggcttgggtgccagctcataaaggaatcggaggaaacgagcaggtggataaactggtgtcttggggctttaccacaccagataagaagcaccagaaggagccaccatttctctggatgggatacgaactgcacccagataagtggacagtccagcctattgtgctgccagaaaaggactcttggacagtgaacgacatccagaaactggtgggaaagctgaattgggcctctcagatctacccaggcatcaaggtgatcgtgatctaccagtacatggacgatctgtacgtgggatcagatctggagatcggacagcacagaatgagggacaattggagaagcgagctgtacaagtacaaggtggtg

SEQ ID NO: 416 - DNA, 1144 bp, synthetic construct
  source 1..1144: mol_type = other DNA
taccagtataacgtgctgcctcagggagcttctagagaactggagagattcgcagtgaacccaggactcctctggattatcctgggactgaacaagatcgtgaggatgtactctcctacctctattgccgctagaacactgaacgcttgggtgaaggtcttcctctggatgggatacgaactgcatctgacctttggttggtgctttaagctccctctctggaaaggaccagctaagctggtgacagtgtattacggagtgccagtggcagctctcctctggaaaggagaaggagcagtggcagcagctaaactggtgggaaagctgaattgggccaaactcctctggaagggagaaggagccaccctgaattttcctatcagccctatttggcaggctacttggattccagagtggaaagcagcttgttggtgggcaggaatcagacaggccaacttcctgggcaagatttggccttctcacaaaggaagaaacgtctgggctacacacgcttgcgtggcagcagaaatgatgacagcttgtcagggagtgtctacagtccagtgtacacacggaatcgcagctaaacagatggcaggagacgattgcgtggcagcttggcagctggattgtacacacctggagtacaaggcagcagtggatctgtctcactttctgagagaaaaaggaggactggaaggagcagcttactacatggacgatctgtacgtgggatcaggacaggtggattgttcaccaggaatcgctacactggaggaaatgatgaccgcagaactgcatccagataagtggaccgtctggacagtgaacgatatccagaagctgggcatttggggttgtagcggaaaactgaccgtgaacgatatccagaagctggtgatcgtgaccgattctcagtacgctctgtacgtggacagattctacaagaccctgtacgtggacaggttctacaagacactgagagccgaacaggcttctcaggaagtggatctgaacaccatgctgaacaccgtgaaactgacacctctctgcgtgacactgtatcagtacatggacgacctgtacgtggtgatctaccagtacatggacgatctctggatcatcctgggactgaacaagatcg

SEQ ID NO: 417 - DNA, 1517 bp, synthetic construct
  source 1..1517: mol_type = other DNA
agaacactgaacgcttgggtgaaggtgagagagaagagagacctgaacaccatgctgaacccgtgagagaaaagaggtggatcatcctgggactgaacaagatcagggagaagaggtacgtggacaggttctacaagacactgagagagaagagagccacactggaagagatgatgaccgctagagagaagagagagatgatgaccgcttgtcagggagtgagagagaagagaaccctgaacttccccatctctcctatcagggagaagaggtaccagtacaacgtgctgcctcagggaagagaaaagagagtgatctaccagtacatggacgacctgagagagaagaggtaccagtacatggacgatctgtacgtgagggagaagagatacatggacgacctgtacgtgggatcaagagagaagagattcctctggatgggctacgagctgcatagagagaagagagagctgcacccagataagtggacagtgagagaaaagcgctggacagtgaacgacatccagaagctgagagagaagaggacagtgaacgacatccagaagctggtgagagagaagaggaagctggtgggaaaactgaattgggctagggaaaaaaggtggcaggctacttggattccagagtggagagagaagaggatcgtgacagatagccagtacgctctgagagagaaaagaggacaggtggattgctctccaggaatcagagagaagagatggcagctggattgtacacacctggagagagagaagaggaaagcagcttgttggtgggcaggaattcgggaaaaaagacctctctggaaaggaccagccaagctgagagagaagagaaaactcctctggaagggcgaaggagctagagaaaagagactcctctggaaaggagaaggcgcagtgagagagaagagaaaacagatggccggagacgattgcgtgagagaaaagagagtgaccgtgtattacggagtgccagtgagagaaaagagaaacgtctgggctacacacgcttgcgtgagagagaagagaaagctgacacctctgtgcgtgacactgagagaaaagagaagcaccgtgcagtgtacacacggaattagggagaagagaggcatttggggttgttcaggaaagctgagagagaagaggctgacattcggttggtgtttcaagctgagggagaagagagcctctagagaactggagagattcgcagtgaatccaggactgctgagagaaaagcgctggattatcctgggactgaacaagatcgtgaggatgtacagccctacaagcatcagagagaagaggtacgtggacagattctacaagaccctgagagccgaacaggcatctcaggaagtgagagagaagagaaggcaggctaacttcctgggaaagatttggcctagccacaagggaagaagagagaagagatacaaggccgcagtggatctgtctcactttctgagagagaaaggaggactggaaggagga

SEQ ID NO: 418 - DNA, 2784 bp, synthetic construct
  source 1..2784: mol_type = other DNA
atggctcctagaagcgctagaagacctctgctgctgctgctgctgctgctgctgctgggactgatgcattgcgcttcagcagctatgttcatggtgaagaacggcaacggaacagcttgtatcatggccaatttcagcgccgcttttagcgtgaattacgacaccaagagcggacctaagaacatgacactggatctgccttcagacgctacagtggtgctgaatagaagctcttgcggaaaggagaatacctccgatccttctctggtgatcgcttttggcagaggacacacactgacactgaacttcaccagaaacgccaccagatactcagtgcagctgatgagcttcgtgtacaacctgagcgatacccatctgtttcctaacgctagcagcaaggagatcaagacagtggagtctatcaccgacatcagagccgatatcgacaagaaataccgctgcgtgtcaggaacacaggtgcacatgaacaacgtgacagtgacactgcacgacgccacaattcaggcctatctgagcaatagcagctttagcagaggcgaaactaggtgtgagcaggatagaccttctcctacaacagctcctccagctcctccttctccttctccttctccagtgcctaaatctcctagcgtggataagtacaacgtgagcggaacaaacggcacttgtctgctggcttctatgggactgcagctgaatctgacatacgagaggaaggacaacaccacagtgacaagactgctgaacatcaaccccaacaaaacaagcgctagcggatcttgcggagctcatctggtgacactggaactgcattcagagggaacaacagtgctgctgtttcagttcggaatgaacgcctctagcagcagattcttcctgcagggtattcagctgaatacactgctgccagatgctagagatccagcctttaaagccgctaatggatctctgagagctctgcaggctacagtgggaaatagctacaagtgcaacgccgaagaacacgtgagagtgacaaaagccttcagcgtgaacatctttaaggtctgggtgcaggcatttaaagtggagggaggccagtttggaagcgtcgaagagtgtctgctggacgaaaatagcctggaagacatcagaacactgaacgcttgggtgaaggtgagagagaagagagacctgaacaccatgctgaacaccgtgagagaaaagaggtggatcatcctgggactgaacaagatcagggagaagaggtacgtggacaggttctacaagacactgagagagaagagagccacactggaagagatgatgaccgctagagagaagagagagatgatgaccgcttgtcagggagtgagagagaagagaaccctgaacttccccatctctcctatcagggagaagaggtaccagtacaacgtgctgcctcagggaagagaaaagagagtgatctaccagtacatggacgacctgagagagaagaggtaccagtacatggacgatctgtacgtgagggagaagagatacatggacgacctgtacgtgggatcaagagagaagagattcctctggatgggctacgagctgcatagagagaagagagagctgcacccagataagtggacagtgagagaaaagcgctggacagtgaacgacatccagaagctgagagagaagaggacagtgaacgacatccagaagctggtgagagagaagaggaagctggtgggaaaactgaattgggctagggaaaaaaggtggcaggctacttggattccagagtggagagagaagaggatcgtgacagatagccagtacgctctgagagagaaaagaggacaggtggattgctctccaggaatcagagagaagagatggcagctggattgtacacacctggagagagagaagaggaaagcagcttgttggtgggcaggaattcgggaaaaaagacctctctggaaaggaccagccaagctgagagagaagagaaaactcctctggaagggcgaaggagctagagaaaagagactcctctggaaaggagaaggcgcagtgagagagaagagaaaacagatggccggagacgattgcgtgagagaaaagagagtgaccgtgtattacggagtgccagtgagagaaaagagaaacgtctgggctacacacgcttgcgtgagagagaagagaaagctgacacctctgtgcgtgacactgagagaaaagagaagcaccgtgcagtgtacacacggaattagggagaagagaggcatttggggttgttcaggaaagctgagagagaagaggctgacattcggttggtgtttcaagctgagggagaagagagcctctagagaactggagagattcgcagtgaatccaggactgctgagagaaaagcgctggattatcctgggactgaacaagatcgtgaggatgtacagccctacaagcatcagagagaagaggtacgtggacagattctacaagaccctgagagccgaacaggcatctcaggaagtgagagagaagagaaggcaggctaacttcctgggaaagatttggcctagccacaagggaagaagagagaagagatacaaggccgcagtggatctgtctcactttctgagagagaaaggaggactggaaggaggaagcgagtttaccctgattccaattgccgtgggaggagctctggcaggactggtgattgtgctgatcgcatacctggtgggaagaaagagatctcacgccggatatcagaccatc

SEQ ID NO: 419-420 - skipped (2 entries)

SEQ ID NO: 421 - protein, 4 aa, synthetic construct
GGGS

SEQ ID NO: 422 - protein, 706 aa, synthetic construct
ICGHKAIGTVLVGPTPVNIIGRNLLTQLGCTLNFPISPIETVPVKLKPGMDGPKVKQWPLTEEKIKALDPLWKGPAKLLWKGEGAVVIQDNSDIKVVPRRKAKIIRDYGKQMAAAYSDIAGTTSTLQEQITWMTNNPPIPVGEIYKRWIILGLNKIVRMYSPVSILDIRQGPKEPFRDYVDRFYKTLRAEQASQEVKNWMTETLLVQNANPDCKTILKALGPAATLEEMMTACQGVGGPGHKARVLAEAMSQVTNSATLNKRTQDFWEVQLGIPHPAGLKKKKSNFTSTTVKAACWWAGIKQEFGIPYNPQSAYFSVPLDKEFRKYTAFTIPSINNEDTVLEEMNLPGKWKPKMIGGIGGFIKVRQYDQISKIGPENPYNTPIFAIKKKDSTKWAAGKKKYRLKHLVWVSRELERFAVNPGGKKKYRLKHLVWASRELERFAVNPGAEHLKTAVQMAVFIHNFKRKGGIGGAAGQMVHQAISPRTLNAWVKVVEEKAFSPEVIPMFSALAEGATPQDLNTMLNTVGGHQARWIILGLNKTVRMYSPVSILDIRQGPKEPFRDYVDRFYKTLRAEQASQEVKNWMTETLLVQNANPDCKTILKALGPAATLEEMMTACQGVGGPGHKARVLAEAMSQVTNSATQLKGEAMHGQVDCSPGIWQLDCTHLEGKVILVAVHVASGYIEAEVIPAETGQETAYFLLKLAGR

SEQ ID NO: 423 - protein, 484 aa, synthetic construct
SNFTSTTVKVACWWAGIKQEFGIPYAASNFTSTTVKAACWWAGVKQEFGIPYAASNFTSTTVKAACWWAGIKQEFGIPYPLRPMTYKAAVDLSHFLKEKGGLEGPLRPMTYKAAVDLSFFLKEKGGLEGPLRPMTYKGAFDLSFFLKEKGGLEGPLRPMTYKAAFDLSFFLKEKGGLEGPLRPMTYKAAFDLSHFLKEKGGLEGPLRPMTYKGALDLSHFLKEKGGLEGQLKGEAMHGQVDCSPGIWQLDCTHLEEKIILVAVHVASGYIEAEVIPAETGQETAYMVHQAISPRTLNAWVKVVEEKAFSPLDCTHLEGKVILVAVHVASGYIEAEICGHKAIGTVLVGPTPVNIIGRNLLTQIGCTLNFPISPIETVPVKLKDPLWKGPAKLLWKGEGAVVIQDNSDIDPLWKGPAKLLWKGEGVVVIQDNSDIMVHQAISPRTLNALVKVVEEKAFSPICGHKAIGTVLVGSTPVNIIGRNLL

SEQ ID NO: 424 - protein, 506 aa, synthetic construct
IRTLNAWVKVREKRDLNTMLNTVREKRWIILGLNKIREKRYVDRFYKTLREKRATLEEMMTAREKREMMTACQGVREKRTLNFPISPIREKRYQYNVLPQGREKRVIYQYMDDLREKRYQYMDDLYVREKRYMDDLYVGSREKRFLWMGYELHREKRELHPDKWTVREKRWTVNDIQKLREKRTVNDIQKLVREKRKLVGKLNWAREKRWQATWIPEWREKRIVTDSQYALREKRGQVDCSPGIREKRWQLDCTHLEREKRKAACWWAGIREKRPLWKGPAKLREKRKLLWKGEGAREKRLLWKGEGAVREKRKQMAGDDCVREKRVTVYYGVPVREKRNVWATHACVREKRKLTPLCVTLREKRSTVQCTHGIREKRGIWGCSGKLREKRLTFGWCFKLREKRASRELERFAVNPGLLREKRWIILGLNKIVRMYSPTSIREKRYVDRFYKTLRAEQASQEVREKRRQANFLGKIWPSHKGRREKRYKAAVDLSHFLREKGGLEG

SEQ ID NO: 425 - protein, 80 aa, unidentified
AVKAACWWAGVKQEFGIPYNTQSQGVVESMNNELKKIIGQIRDQAEHLKTAVQMAVLIHNFKRKGGIGEYSAGERIIDII

SEQ ID NO: 426 - protein, 81 aa, unidentified
AVKAACWWAGVKQEFGIPYNTQSQGVVESMNNELKKIIGQIRDQAEHLKTAVQMAVLIHNFKRKGGIGEYSAGERIIDIIA

SEQ ID NO: 427 - protein, 81 aa, unidentified
VAKEIVACCDKCQLKGEAIHGQVDCSPGVWQLDCTHLEGKVILVAVHVASGYIEAEIIPTETGQETAYFILKLAGRWPVTT

SEQ ID NO: 428 - protein, 256 aa, unidentified
LKHIVWASRELERFAVNPGLLETVSQNYPIVQNISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQ

SEQ ID NO: 429 - protein, 218 aa, synthetic construct
LPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSWGFTTPDKKHQKEPPFLWMGYELHPDKWTVQPI

SEQ ID NO: 430 - protein, 368 aa, synthetic construct
MGARASVLSGGELDRWEKIRLRPGGKKKYRLKHIVWASRELERFAVNPGLLETLKHIVWASRELERFAVNPGLLETAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQEEVGFPVKPQVPLRPMTFKGALDLSHFLREKGGLEGTQGFFPDQNYTPEPGIRFPLTFGWCFKLVPL

SEQ ID NO: 431 - protein, 381 aa, synthetic construct
YQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLYVDRFYKTLRAEQASQEVDLNTMLNTVKLTPLCVTLYQYMDDLYVVIYQYMDDLWIILGLNKI

SEQ ID NO: 432 - protein, 399 aa, synthetic construct
MWLQSLLLLGTVACSISVYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLYVDRFYKTLRAEQASQEVDLNTMLNTVKLTPLCVTLYQYMDDLYVVIYQYMDDLWIILGLNKI

SEQ ID NO: 433 - protein, 404 aa, synthetic construct
MDAMKRGLCCVLLLCGAVFVSARYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLYVDRFYKTLRAEQASQEVDLNTMLNTVKLTPLCVTLYQYMDDLYVVIYQYMDDLWIILGLNKI

SEQ ID NO: 434 - protein, 488 aa, synthetic construct
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLYVDRFYKTLRAEQASQEVDLNTMLNTVKLTPLCVTLYQYMDDLYVVIYQYMDDLWIILGLNKI

SEQ ID NO: 435 - protein, 412 aa, synthetic construct
MRKAAVSHWQQQSYLDSGIHSGATTTAPSLSYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLYVDRFYKTLRAEQASQEVDLNTMLNTVKLTPLCVTLYQYMDDLYVVIYQYMDDLWIILGLNKI

SEQ ID NO: 436 - protein, 9 aa, synthetic construct
DYKDDDDKL

SEQ ID NO: 437 - protein, 27 aa, Human immunodeficiency virus 1
MRVKEKYQHLWRWGWRWGTMLLGMLMI

SEQ ID NO: 438 - protein, 6 aa, Human immunodeficiency virus 1
FCASDA

SEQ ID NO: 439 - protein, 29 aa, Human immunodeficiency virus 1
VVLVNVTENFNMWKNDMVEQMHEDIISLW

SEQ ID NO: 440 - protein, 96 aa, Human immunodeficiency virus 1
NTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFN

SEQ ID NO: 441 - protein, 206 aa, Human immunodeficiency virus 1
EVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGD

SEQ ID NO: 442 - protein, 12 aa, Human immunodeficiency virus 1
KIEPLGVAPTKA

SEQ ID NO: 443 - protein, 246 aa, Human immunodeficiency virus 1
NASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 444 - protein, 30 aa, Human immunodeficiency virus 1
MGARASVLSGGELDRWEKIRLRPGGKKKYK

SEQ ID NO: 445 - protein, 74 aa, Human immunodeficiency virus 1
SEGCRQILGQLQPSLQTGSEELRSLYNTVATLYCVHQRIEIKDTKEALDKIEEEQNKSKKKAQQAAADTGHSNQ

SEQ ID NO: 446 - protein, 9 aa, Human immunodeficiency virus 1
IQGQMVHQA

SEQ ID NO: 447 - protein, 59 aa, Human immunodeficiency virus 1
VTNSATIMMQRGNFRNQRKIVKCFNCGKEGHTARNCRAPRKKGCWKCGKEGHQMKDCTE

SEQ ID NO: 448 - protein, 56 aa, Human immunodeficiency virus 1
PGNFLQSRPEPTAPPEESFRSGVETTTPPQKQEPIDKELYPLTSLRSLFGNDPSSQ

SEQ ID NO: 449 - protein, 63 aa, Human immunodeficiency virus 1
MGGKWSKSSVIGWPTVRERMRRAEPAADRVGAASRDLEKHGAITSSNTAATNAACAWLEAQEE

SEQ ID NO: 450 - protein, 17 aa, Human immunodeficiency virus 1
LIHSQRRQDILDLWIYH

SEQ ID NO: 451 - protein, 76 aa, Human immunodeficiency virus 1
PGVRYPLTFGWCYKLVPVEPDKIEEANKGENTSLLHPVSLHGMDDPEREVLEWRFDSRLAFHHVARELHPEYFKNC

SEQ ID NO: 452 - protein, 55 aa, Human immunodeficiency virus 1
FFREDLAFLQGKAREFSSEQTRANSPTRRELQVWGRDNNSPSEAGADRQGTVSFN

SEQ ID NO: 453 - protein, 11 aa, Human immunodeficiency virus 1
ILIEICGHKAI

SEQ ID NO: 454 - protein, 5 aa, Human immunodeficiency virus 1
KILEP

SEQ ID NO: 455 - protein, 12 aa, Human immunodeficiency virus 1
TKIEELRQHLLR

SEQ ID NO: 456 - protein, 109 aa, Human immunodeficiency virus 1
QLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKT

SEQ ID NO: 457 - protein, 35 aa, Human immunodeficiency virus 1
LGKAGYVTNRGRQKVVTLTDTTNQKTELQAIYLAL

SEQ ID NO: 458 - protein, 16 aa, Human immunodeficiency virus 1
QSESELVNQIIEQLIK

SEQ ID NO: 459 - protein, 37 aa, Human immunodeficiency virus 1
GIRKVLFLDGIDKAQDEHEKYHSNWRAMASDFNLPPV

SEQ ID NO: 460 - protein, 6 aa, Human immunodeficiency virus 1
IHTDNG

SEQ ID NO: 461 - protein, 10 aa, Human immunodeficiency virus 1
TDIQTKELQK

SEQ ID NO: 462 - protein, 4 aa, synthetic construct
YMDD

SEQ ID NO: 463 - protein, 4 aa, synthetic construct
YVDD

SEQ ID NO: 464 - protein, 9 aa, synthetic construct
AIIIIIIIS

SEQ ID NO: 465 - protein, 9 aa, synthetic construct
MIIIIIIII

SEQ ID NO: 466 - protein, 9 aa, synthetic construct
IIIIIIISK

SEQ ID NO: 467 - protein, 9 aa, synthetic construct
IIIIIIIIR

SEQ ID NO: 468 - protein, 10 aa, synthetic construct
AIIIIIIISK

SEQ ID NO: 469 - protein, 10 aa, synthetic construct
MIIIIIIIIR

SEQ ID NO: 470 - protein, 4 aa, synthetic construct
  VARIANT 1..4: note = This sequence may encompass 2-4 residues
AAAA

SEQ ID NO: 471 - protein, 10 aa, synthetic construct
GIIIIIIIIK

SEQ ID NO: 472 - protein, 10 aa, synthetic construct
AIIIIIIIIH

SEQ ID NO: 473 - protein, 10 aa, synthetic construct
GIIIIIIIIH

SEQ ID NO: 474 - protein, 10 aa, synthetic construct
AIIIIIIIIK

SEQ ID NO: 475 - protein, 27 aa, synthetic construct
QNLQGQMVHQAISPRTLNAWVKVVKAF

SEQ ID NO: 476 - protein, 27 aa, synthetic construct
QNLQGQMVHQAISPRTLNAWVKVEKAF

SEQ ID NO: 477 - protein, 27 aa, synthetic construct
QNLQGQMVHQAISPRTLNAWVKIVKAF

SEQ ID NO: 478 - protein, 27 aa, synthetic construct
QNIQGQMVHQAISPRTLNAWVKIEKAF

SEQ ID NO: 479 - protein, 27 aa, synthetic construct
QNIQGQMVHQAISPRTLNAWVKVVKAF

SEQ ID NO: 480 - protein, 27 aa, synthetic construct
PNIQGQMVHQAISPRTLNAWVKIVKAF

SEQ ID NO: 481 - protein, 9 aa, synthetic construct
QNLQGQMVH

SEQ ID NO: 482 - protein, 9 aa, synthetic construct
QNIQGQMVH

SEQ ID NO: 483 - protein, 9 aa, synthetic construct
PNIQGQMVH

SEQ ID NO: 484 - protein, 10 aa, synthetic construct
GIIIIIIIIR

SEQ ID NO: 485 - protein, 9 aa, synthetic construct
GIIIIIIII

SEQ ID NO: 486 - protein, 9 aa, synthetic construct
AIIIIIIII

SEQ ID NO: 487 - protein, 9 aa, synthetic construct
IIIIIIIIK

SEQ ID NO: 488 - protein, 9 aa, synthetic construct
IIIIIIIIH

SEQ ID NO: 489 - protein, 9 aa, unidentified
QAISPRTLN

SEQ ID NO: 490 - protein, 27 aa, unidentified
QNLQGQMVHQATSPRTLNAWVKVVKAF

SEQ ID NO: 491 - protein, 9 aa, unidentified
QATSPRTLN

SEQ ID NO: 492 - protein, 9 aa, Human immunodeficiency virus 1
HPPQAGPVA

SEQ ID NO: 493 - protein, 9 aa, Homo sapiens
VPLQAGPVQ

SEQ ID NO: 494 - protein, 9 aa, Homo sapiens
MPGQAGPVG

SEQ ID NO: 495 - protein, 9 aa, Homo sapiens
QYRQAGPVI

SEQ ID NO: 496 - protein, 9 aa, Homo sapiens
TATQAGLVF

SEQ ID NO: 497 - protein, 9 aa, Homo sapiens
HTKQAGLVV

SEQ ID NO: 498 - protein, 9 aa, Homo sapiens
GTRQAGLVA

SEQ ID NO: 499 - protein, 9 aa, unidentified
ISPRTLNAW

SEQ ID NO: 500 - protein, 9 aa, unidentified
MFSALSEGA

SEQ ID NO: 501 - protein, 9 aa, unidentified
ILDIRQGPK

SEQ ID NO: 502 - protein, 9 aa, unidentified
LSPRTLNAW

SEQ ID NO: 503 - protein, 9 aa, unidentified
MFSALSYGA

SEQ ID NO: 504 - protein, 9 aa, unidentified
ILDIRRGPK

SEQ ID NO: 505 - protein, 9 aa, unidentified
ATLEEMVTA

SEQ ID NO: 506 - protein, 9 aa, unidentified
ILDTRQGPK

SEQ ID NO: 507 - protein, 9 aa, unidentified
ATLVEMMTA

SEQ ID NO: 508 - protein, 9 aa, unidentified
ILSIRQGPK

SEQ ID NO: 509 - protein, 9 aa, unidentified
ATSEEMMTA

SEQ ID NO: 510 - protein, 9 aa, unidentified
  VARIANT 8: note = Any amino acid
ILDIRQGXK

SEQ ID NO: 511 - protein, 109 aa, unidentified
KRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTAVPW

SEQ ID NO: 512 - protein, 21 aa, unidentified
YLKDQQLLGIWGCSGKLICTT

SEQ ID NO: 513 - protein, 35 aa, unidentified
EEVGFPVKPQVPLRPMTFKGALDLSHFLREKGGLE

SEQ ID NO: 514 - protein, 14 aa, unidentified
TQGFFPDWQNYTPE

SEQ ID NO: 515 - protein, 18 aa, unidentified
PGIRFPLTFGWCFKLVPL

SEQ ID NO: 516 - protein, 21 aa, unidentified
VTVLDVGDAYFSVPLDKDFRK

SEQ ID NO: 517 - protein, 8 aa, unidentified
YTAFTIPS

SEQ ID NO: 518 - protein, 8 aa, unidentified
VIYQYMDD

SEQ ID NO: 519 - protein, 78 aa, unidentified
TVKAACWWAGIKQEFGIPYNPQSQGVVESMNKELKKIIGQVRDQAEHLKTAVQMAVFIHNFKRKGGIGGYSAGERIVD

SEQ ID NO: 520 - DNA, 32331 bp, synthetic construct
  source 1..32331: mol_type = other DNA
catcatcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagtagggataacagggtaatgacattgattattgactagttgttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggaacgccatccacgctgttttgacctccatagaagacagcgatcgcgccaccatgatttctcctcggacgctgaatgcatgggtgaaggtagtggaggaaaaggcattttcaccagaagtcattccgatgttctccgcgctctccgagggtgctacgccacaggacttgaatacgatgctgaataccgttggtggccatcaggcggcgatgcagatgttgaaggagacaattaacgaagaagccgccgaatgggacagattgcacccggtgcatgcggggccaatagctcctggccagatgcgcgagcctaggggttctgacattgctggtacaacaagtacccttcaggagcagattggttggatgacgaataaccctcccatacctgttggcgagatctataagcgctggattatacttgggctgaataagatagtccgaatgtattctcccacctctattctggatattcggcaaggacctaaggagccgtttagagactacgtagaccggttttacaaaaccctgcgggcggaacaagcttctcaggaagtcaaaaattggatgactgagaccttgctcgtccagaatgcgaacccggactgtaaaacaatactcaaagcgctgggccccgctgcaaccctggaagaaatgatgacggcttgtcagggagtaggaggccccggacataaggcacgagtgttggcagaagccatgagccagccgcctgtcgtcgcgaaagaaatcgtcgcttcttgcgacaaatgtcagctgaagggggaggcgatgcacggtcaagttgattgctctcccggtatttggcaattggactgtacccaccttgaaggcaaaattattctggttgcagtgcacgtagcatccggttacatcgaagctgaagtgatacccgcagagacaggccaggagacggcttatttcctccttaagcttgcgggtcggtggcccgtaaagaccatggctgctcgggcatctgtcctctccggtggtgaactcgaccgatgggaaaagattcgattgcgccccggaggaaagaagaaatataggctgaaacatattgtgtgggcatcacgggaacttgagcgatttgcggtaaacccaggccttttggaaacagctgcaactgtgaaagcggcttgctggtgggcggggataaaacaggagtttggtatcccgtacaatccccaatctcagggggtagtagaaagcatgaacaaagaattgaaaaaaataattggccaggttcgcgaccaagccgagcacctcaagaccgctgtacagatggctgtatttattcacaacttcaagcggaagggcggaataggaggatatagcgcaggggaaaggattgttgatattattgcaacacaaggttactttcctgactggcaaaactacacaccgggccctggcacgcgctatccccttacgttcggttggtgcttcaagctggtgccggtaaaagaaaaagtttatttggcatgggttcctgcacataaaggaatagggggtaacgaacaagttgacaaactcgtcagccgcgctaaaagagccccagtcaagcagaccctgaattttgacctgcttaaattggctggggacgtcgagagtaacccgggacccttcccacaaattacactctggcagcgaccactggtaacaatcaaaatagggggacaattgaaagaagcactcctggatacgggcgcggacgatacagtcctggaggaaatgaatctccccggccgctggaaacctaagatgataggggggatcggtggatttattaaagtgcggcagtacgatcaatggggttttacgacacctgacaagaaacatcaaaaggagccgccatttctttggatgggttatgagcttcatccggataaatggactgttcagccgattgtcctccccgagaaggatagttggactgtgaacgacatccagaagctggtcgggaaacttaattgggccagtcaaatatatccaggtattaaagttgccgcacaagaagaggaggaagtagggttccccgtgaaaccgcaagtccctctccggcccatgacctttaagggcgctctcgacctgtcccatttcctccgcgaaaaggggggtttggaaggcttgatcattaccaagattcaaaacttcagggtctattatcgagacagtcgcgatcccctttggaaaggacctgcgaaacttctttggaaaggagaaggagccgtggtaattcaggataattctgacataaaggtcgtcccacgccgaaaagcgaagattataagagattatggcaagcagatggccggggatgattgtgtcgcaagtagacaggatgaagaccctaaattcaagcttccaatccagaaggaaacgtgggagacatggtggaccgagtattggcaagctacttggatcccagaatgggaatttgtgaacactcccccactcgtaaagctgtggtaccagcttgaaaaagaacctatagtcggggcggagacgttctatgtagacggcgccgctaatcgagagacaaaagcagcaggcacggtactggtaggcccgacccctgtcaacatcattggacgaaatctgttgacccagattgggtgtacccttaactttcccatttcaccaatagagaccgtcccggttaaactgaaaccgggtatggatggtcccaaagtaaaacagtggccacttaccgaggagaagattaaggcactcgttgaaatatgtacagaaatggaaaaagaggggaaaatctctaaaattggccctgaaaatccgtacaacactccggtattcgccataaaaaagaaggactctaccaagtggcgcaaactcgtggactttagagaactgaataaaaggacccaggacttttgggaagtccagctgggtattcctcaccccgctggtctcaagaaaaagaaaagtgtcactgtcttggatgttggagatgcgtacttttcagtacctcttgataaagattttcgaaagtataccgcgtttaccattccctccataaataacgaaacaccggggatcaggtatcaatataacgtgcttccacaaggctggaagggttcaccggctattttccaatcttctatgacgtgataggtttaaactcccatttaaatgtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataactataacggtcctaaggtagcgagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatgaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatg

SEQ ID NO: 521 - DNA, 32328 bp, synthetic construct
  source 1..32328: mol_type = other DNA
catcatcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagtagggataacagggtaatgacattgattattgactagttgttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggaacgccatccacgctgttttgacctccatagaagacagcgatcgcgccaccatgttgtctcctagaactctgaatgcttgggtaaaggtgatcgaagaaaaggcattctcacccgaggttatccctatgtttactgcgttgagcgaaggcgcaacaccccatgatctgaacacaatgctgaatacaatcggcggacatcaagctgctatgcaaatgcttaaggacaccatcaatgaggaggcagccgagtgggatcgcgttcatccagtccacgctgggcccgttgcgcctggtcagatgagggacccacgaggatccgacatcgcagggagcaccagtacactccaagagcagattgcatggatgacgaacaatccaccaatacctgtcggtgacatttacaaaaggtggattattatgggcttgaacaaaattgtacggatgtatagcccggtgagcatactggacattaaacagggtccaaaagaaccctttcgagattacgttgacagattttataggacactgagggcggaacaagcgtctcaagacgttaaaaactggatgacagaaacgctgcttgtacagaattccaacccagactgcaaaactatactgaaagcgctcggtcccggtgcgacacttgaggaaatgatgagcgcatgccagggcgtcggagggccgtctcacaaggcccgcgtgttggcggaagctatgtgccaggtggctaaggagatagtagcatgttgtgataaatgtcagcttaaaggtgaagctatacatgggcaggtggattgtagcccgggtgtatggcagctggactgtactcatctggaaggaaaggtaatacttgtcgcagttcatgtcgcgagcggatacattgaagctgaaatcattcctacggagacgggccaagagacagcttacttcatacttaaacttgctgggcgatggccggtgacaactatggcggcgcgcgcttcaattttgagtggtggaaagttggacaagtgggagaagattagactcagacccggagggagaaagaagtataaactgaaacatctggtttgggcttcacgcgaacttgaacggtttgctctcaaccccgggctgctcgaaactgctgctgctgtaaaggccgcttgttggtgggcgggggtaaagcaggaattcggaattccatataacactcaaagccagggagtagtggaatccatgaataatgaacttaagaagataattggacagattcgcgatcaggctgaacatctcaagacggccgtacaaatggcagtattgattcataactttaaacggaagggcggcataggagagtattctgcgggagaacgcataatagatataattgcgactcaggggttctttccggattggcagaactatacgccggggccaggcattaggttccccctcacgtttggatggtgtttcaagttggtaccgttgctcattaaaaaagaaaaaatctacctggcctgggtcccggcgcacaagggtataggggggaacgagcaaattgataagctcgtgtcaagggcgaagcgcgcgccagtcaaacagaccttgaatttcgacctccttaagctcgctggagacgtcgaatccaaccctggcccgatgctgccacaaatcacattgtggcaacgacccattgtaacaataaaaatcgggggccagatcaaagaagcgctgcttgacaccggcgccgacgatacagtcctcgaggatatgaatttgccaggcaaatggaagccgaagatgattggcggcattggcggctttattaaggttaaacagtatgatcagtggggattgaccactcccgataagaagcatcagaaagatccgccttttctgtggatggggtacgaactgcaccctgatcgatggacggtccagccgatagagctgccggaaaaagaatcatggaccgtgaatgatattcaaaaactgatcggaaaactcaattgggcgtcccagatatatgctggcatcaaagttgcagcacaagaagaggaagaggtaggtttcccggttcggccgcaagttcccttgcgaccgatgacatacaagggcgcattggacctttctcacttcctcaaggaaaagggcggtttggagggcatcactaaacttcagaatttcagagtctactatagagataacagggacccattgtggaagggccccgctcgacttctctggaaaggggagggagcggttgtaattcaagacaacagtgaaattaaggtcgtcccacgacggaaggttaaaataattcgcgactatggcaagcgaatggcgggagacgactgtgtagcaggacgacaagacgaggacccaaagtttagattgccgatccagaaagagacatgggatacgtggtggacggactattggcaggccacctggataccagaatgggagtttacaaacactcctccactcgtgaaattgtggtatcaacttgagaccgagcccatagctggtgtagagacgttttacgttgacggtgctagcaacagggaaacaaaggccgctggaaccgtgctcatcggtcctactcctgtgaacataattggacgaaatttgttgacccagctgggatgcaccctcaatttccccattagcccaatagataccgtaccagtcaagcttaagcctggtatggacggtccgcgagttaagcaatggccacttactgaggagaaaatcaaggcactcatcgagatctgcaccgaaatggagaaggagggcaaaataagcaggattggtcccgagaatccatataatacgccgatcttcgcgataaaaaagaaggacggcaccaaatggcgaaaactggttgacttccgggagcttaacaaaaaaactcaggatttttgggaagttcaactcgggatcccacacccgtctggtcttaaaaaaaaaaaaagcgtaacagtccttgacatcggcgatgcctactttagcgtgcctctcgataaggagttcagaaaatacacggctttcactgtaccaagcacaaacaatgagactcctggggtcagataccagtacaatgtccttcccatggggtggaaaggaagccccgcaatattccagtgctcaatgacgtgataggtttaaactcccatttaaatgtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataactataacggtcctaaggtagcgagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatgaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatg

SEQ ID NO: 522 - DNA, 11072 bp, synthetic construct
  source 1..11072: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactctagaatagtctttaattaagccaccatgatttctcctcggacgctgaatgcatgggtgaaggtagtggaggaaaaggcattttcaccagaagtcattccgatgttctccgcgctctccgagggtgctacgccacaggacttgaatacgatgctgaataccgttggtggccatcaggcggcgatgcagatgttgaaggagacaattaacgaagaagccgccgaatgggacagattgcacccggtgcatgcggggccaatagctcctggccagatgcgcgagcctaggggttctgacattgctggtacaacaagtacccttcaggagcagattggttggatgacgaataaccctcccatacctgttggcgagatctataagcgctggattatacttgggctgaataagatagtccgaatgtattctcccacctctattctggatattcggcaaggacctaaggagccgtttagagactacgtagaccggttttacaaaaccctgcgggcggaacaagcttctcaggaagtcaaaaattggatgactgagaccttgctcgtccagaatgcgaacccggactgtaaaacaatactcaaagcgctgggccccgctgcaaccctggaagaaatgatgacggcttgtcagggagtaggaggccccggacataaggcacgagtgttggcagaagccatgagccagccgcctgtcgtcgcgaaagaaatcgtcgcttcttgcgacaaatgtcagctgaagggggaggcgatgcacggtcaagttgattgctctcccggtatttggcaattggactgtacccaccttgaaggcaaaattattctggttgcagtgcacgtagcatccggttacatcgaagctgaagtgatacccgcagagacaggccaggagacggcttatttcctccttaagcttgcgggtcggtggcccgtaaagaccatggctgctcgggcatctgtcctctccggtggtgaactcgaccgatgggaaaagattcgattgcgccccggaggaaagaagaaatataggctgaaacatattgtgtgggcatcacgggaacttgagcgatttgcggtaaacccaggccttttggaaacagctgcaactgtgaaagcggcttgctggtgggcggggataaaacaggagtttggtatcccgtacaatccccaatctcagggggtagtagaaagcatgaacaaagaattgaaaaaaataattggccaggttcgcgaccaagccgagcacctcaagaccgctgtacagatggctgtatttattcacaacttcaagcggaagggcggaataggaggatatagcgcaggggaaaggattgttgatattattgcaacacaaggttactttcctgactggcaaaactacacaccgggccctggcacgcgctatccccttacgttcggttggtgcttcaagctggtgccggtaaaagaaaaagtttatttggcatgggttcctgcacataaaggaatagggggtaacgaacaagttgacaaactcgtcagccgcgctaaaagagccccagtcaagcagaccctgaattttgacctgcttaaattggctggggacgtcgagagtaacccgggacccttcccacaaattacactctggcagcgaccactggtaacaatcaaaatagggggacaattgaaagaagcactcctggatacgggcgcggacgatacagtcctggaggaaatgaatctccccggccgctggaaacctaagatgataggggggatcggtggatttattaaagtgcggcagtacgatcaatggggttttacgacacctgacaagaaacatcaaaaggagccgccatttctttggatgggttatgagcttcatccggataaatggactgttcagccgattgtcctccccgagaaggatagttggactgtgaacgacatccagaagctggtcgggaaacttaattgggccagtcaaatatatccaggtattaaagttgccgcacaagaagaggaggaagtagggttccccgtgaaaccgcaagtccctctccggcccatgacctttaagggcgctctcgacctgtcccatttcctccgcgaaaaggggggtttggaaggcttgatcattaccaagattcaaaacttcagggtctattatcgagacagtcgcgatcccctttggaaaggacctgcgaaacttctttggaaaggagaaggagccgtggtaattcaggataattctgacataaaggtcgtcccacgccgaaaagcgaagattataagagattatggcaagcagatggccggggatgattgtgtcgcaagtagacaggatgaagaccctaaattcaagcttccaatccagaaggaaacgtgggagacatggtggaccgagtattggcaagctacttggatcccagaatgggaatttgtgaacactcccccactcgtaaagctgtggtaccagcttgaaaaagaacctatagtcggggcggagacgttctatgtagacggcgccgctaatcgagagacaaaagcagcaggcacggtactggtaggcccgacccctgtcaacatcattggacgaaatctgttgacccagattgggtgtacccttaactttcccatttcaccaatagagaccgtcccggttaaactgaaaccgggtatggatggtcccaaagtaaaacagtggccacttaccgaggagaagattaaggcactcgttgaaatatgtacagaaatggaaaaagaggggaaaatctctaaaattggccctgaaaatccgtacaacactccggtattcgccataaaaaagaaggactctaccaagtggcgcaaactcgtggactttagagaactgaataaaaggacccaggacttttgggaagtccagctgggtattcctcaccccgctggtctcaagaaaaagaaaagtgtcactgtcttggatgttggagatgcgtacttttcagtacctcttgataaagattttcgaaagtataccgcgtttaccattccctccataaataacgaaacaccggggatcaggtatcaatataacgtgcttccacaaggctggaagggttcaccggctattttccaatcttctatgacgtgatagttcgaacggccgtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 523 - DNA, 11069 bp, synthetic construct
  source 1..11069: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactctagaatagtctttaattaagccaccatgttgtctcctagaactctgaatgcttgggtaaaggtgatcgaagaaaaggcattctcacccgaggttatccctatgtttactgcgttgagcgaaggcgcaacaccccatgatctgaacacaatgctgaatacaatcggcggacatcaagctgctatgcaaatgcttaaggacaccatcaatgaggaggcagccgagtgggatcgcgttcatccagtccacgctgggcccgttgcgcctggtcagatgagggacccacgaggatccgacatcgcagggagcaccagtacactccaagagcagattgcatggatgacgaacaatccaccaatacctgtcggtgacatttacaaaaggtggattattatgggcttgaacaaaattgtacggatgtatagcccggtgagcatactggacattaaacagggtccaaaagaaccctttcgagattacgttgacagattttataggacactgagggcggaacaagcgtctcaagacgttaaaaactggatgacagaaacgctgcttgtacagaattccaacccagactgcaaaactatactgaaagcgctcggtcccggtgcgacacttgaggaaatgatgagcgcatgccagggcgtcggagggccgtctcacaaggcccgcgtgttggcggaagctatgtgccaggtggctaaggagatagtagcatgttgtgataaatgtcagcttaaaggtgaagctatacatgggcaggtggattgtagcccgggtgtatggcagctggactgtactcatctggaaggaaaggtaatacttgtcgcagttcatgtcgcgagcggatacattgaagctgaaatcattcctacggagacgggccaagagacagcttacttcatacttaaacttgctgggcgatggccggtgacaactatggcggcgcgcgcttcaattttgagtggtggaaagttggacaagtgggagaagattagactcagacccggagggagaaagaagtataaactgaaacatctggtttgggcttcacgcgaacttgaacggtttgctctcaaccccgggctgctcgaaactgctgctgctgtaaaggccgcttgttggtgggcgggggtaaagcaggaattcggaattccatataacactcaaagccagggagtagtggaatccatgaataatgaacttaagaagataattggacagattcgcgatcaggctgaacatctcaagacggccgtacaaatggcagtattgattcataactttaaacggaagggcggcataggagagtattctgcgggagaacgcataatagatataattgcgactcaggggttctttccggattggcagaactatacgccggggccaggcattaggttccccctcacgtttggatggtgtttcaagttggtaccgttgctcattaaaaaagaaaaaatctacctggcctgggtcccggcgcacaagggtataggggggaacgagcaaattgataagctcgtgtcaagggcgaagcgcgcgccagtcaaacagaccttgaatttcgacctccttaagctcgctggagacgtcgaatccaaccctggcccgatgctgccacaaatcacattgtggcaacgacccattgtaacaataaaaatcgggggccagatcaaagaagcgctgcttgacaccggcgccgacgatacagtcctcgaggatatgaatttgccaggcaaatggaagccgaagatgattggcggcattggcggctttattaaggttaaacagtatgatcagtggggattgaccactcccgataagaagcatcagaaagatccgccttttctgtggatggggtacgaactgcaccctgatcgatggacggtccagccgatagagctgccggaaaaagaatcatggaccgtgaatgatattcaaaaactgatcggaaaactcaattgggcgtcccagatatatgctggcatcaaagttgcagcacaagaagaggaagaggtaggtttcccggttcggccgcaagttcccttgcgaccgatgacatacaagggcgcattggacctttctcacttcctcaaggaaaagggcggtttggagggcatcactaaacttcagaatttcagagtctactatagagataacagggacccattgtggaagggccccgctcgacttctctggaaaggggagggagcggttgtaattcaagacaacagtgaaattaaggtcgtcccacgacggaaggttaaaataattcgcgactatggcaagcgaatggcgggagacgactgtgtagcaggacgacaagacgaggacccaaagtttagattgccgatccagaaagagacatgggatacgtggtggacggactattggcaggccacctggataccagaatgggagtttacaaacactcctccactcgtgaaattgtggtatcaacttgagaccgagcccatagctggtgtagagacgttttacgttgacggtgctagcaacagggaaacaaaggccgctggaaccgtgctcatcggtcctactcctgtgaacataattggacgaaatttgttgacccagctgggatgcaccctcaatttccccattagcccaatagataccgtaccagtcaagcttaagcctggtatggacggtccgcgagttaagcaatggccacttactgaggagaaaatcaaggcactcatcgagatctgcaccgaaatggagaaggagggcaaaataagcaggattggtcccgagaatccatataatacgccgatcttcgcgataaaaaagaaggacggcaccaaatggcgaaaactggttgacttccgggagcttaacaaaaaaactcaggatttttgggaagttcaactcgggatcccacacccgtctggtcttaaaaaaaaaaaaagcgtaacagtccttgacatcggcgatgcctactttagcgtgcctctcgataaggagttcagaaaatacacggctttcactgtaccaagcacaaacaatgagactcctggggtcagataccagtacaatgtccttcccatggggtggaaaggaagccccgcaatattccagtgctcaatgacgtgatagttcgaacggccgtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 524 - DNA, 3096 bp, synthetic construct
  source 1..3096: mol_type = other DNA
atgatttctcctcggacgctgaatgcatgggtgaaggtagtggaggaaaaggcattttcaccagaagtcattccgatgttctccgcgctctccgagggtgctacgccacaggacttgaatacgatgctgaataccgttggtggccatcaggcggcgatgcagatgttgaaggagacaattaacgaagaagccgccgaatgggacagattgcacccggtgcatgcggggccaatagctcctggccagatgcgcgagcctaggggttctgacattgctggtacaacaagtacccttcaggagcagattggttggatgacgaataaccctcccatacctgttggcgagatctataagcgctggattatacttgggctgaataagatagtccgaatgtattctcccacctctattctggatattcggcaaggacctaaggagccgtttagagactacgtagaccggttttacaaaaccctgcgggcggaacaagcttctcaggaagtcaaaaattggatgactgagaccttgctcgtccagaatgcgaacccggactgtaaaacaatactcaaagcgctgggccccgctgcaaccctggaagaaatgatgacggcttgtcagggagtaggaggccccggacataaggcacgagtgttggcagaagccatgagccagccgcctgtcgtcgcgaaagaaatcgtcgcttcttgcgacaaatgtcagctgaagggggaggcgatgcacggtcaagttgattgctctcccggtatttggcaattggactgtacccaccttgaaggcaaaattattctggttgcagtgcacgtagcatccggttacatcgaagctgaagtgatacccgcagagacaggccaggagacggcttatttcctccttaagcttgcgggtcggtggcccgtaaagaccatggctgctcgggcatctgtcctctccggtggtgaactcgaccgatgggaaaagattcgattgcgccccggaggaaagaagaaatataggctgaaacatattgtgtgggcatcacgggaacttgagcgatttgcggtaaacccaggccttttggaaacagctgcaactgtgaaagcggcttgctggtgggcggggataaaacaggagtttggtatcccgtacaatccccaatctcagggggtagtagaaagcatgaacaaagaattgaaaaaaataattggccaggttcgcgaccaagccgagcacctcaagaccgctgtacagatggctgtatttattcacaacttcaagcggaagggcggaataggaggatatagcgcaggggaaaggattgttgatattattgcaacacaaggttactttcctgactggcaaaactacacaccgggccctggcacgcgctatccccttacgttcggttggtgcttcaagctggtgccggtaaaagaaaaagtttatttggcatgggttcctgcacataaaggaatagggggtaacgaacaagttgacaaactcgtcagccgcgctaaaagagccccagtcaagcagaccctgaattttgacctgcttaaattggctggggacgtcgagagtaacccgggacccttcccacaaattacactctggcagcgaccactggtaacaatcaaaatagggggacaattgaaagaagcactcctggatacgggcgcggacgatacagtcctggaggaaatgaatctccccggccgctggaaacctaagatgataggggggatcggtggatttattaaagtgcggcagtacgatcaatggggttttacgacacctgacaagaaacatcaaaaggagccgccatttctttggatgggttatgagcttcatccggataaatggactgttcagccgattgtcctccccgagaaggatagttggactgtgaacgacatccagaagctggtcgggaaacttaattgggccagtcaaatatatccaggtattaaagttgccgcacaagaagaggaggaagtagggttccccgtgaaaccgcaagtccctctccggcccatgacctttaagggcgctctcgacctgtcccatttcctccgcgaaaaggggggtttggaaggcttgatcattaccaagattcaaaacttcagggtctattatcgagacagtcgcgatcccctttggaaaggacctgcgaaacttctttggaaaggagaaggagccgtggtaattcaggataattctgacataaaggtcgtcccacgccgaaaagcgaagattataagagattatggcaagcagatggccggggatgattgtgtcgcaagtagacaggatgaagaccctaaattcaagcttccaatccagaaggaaacgtgggagacatggtggaccgagtattggcaagctacttggatcccagaatgggaatttgtgaacactcccccactcgtaaagctgtggtaccagcttgaaaaagaacctatagtcggggcggagacgttctatgtagacggcgccgctaatcgagagacaaaagcagcaggcacggtactggtaggcccgacccctgtcaacatcattggacgaaatctgttgacccagattgggtgtacccttaactttcccatttcaccaatagagaccgtcccggttaaactgaaaccgggtatggatggtcccaaagtaaaacagtggccacttaccgaggagaagattaaggcactcgttgaaatatgtacagaaatggaaaaagaggggaaaatctctaaaattggccctgaaaatccgtacaacactccggtattcgccataaaaaagaaggactctaccaagtggcgcaaactcgtggactttagagaactgaataaaaggacccaggacttttgggaagtccagctgggtattcctcaccccgctggtctcaagaaaaagaaaagtgtcactgtcttggatgttggagatgcgtacttttcagtacctcttgataaagattttcgaaagtataccgcgtttaccattccctccataaataacgaaacaccggggatcaggtatcaatataacgtgcttccacaaggctggaagggttcaccggctattttccaatcttctatgacgtga

SEQ ID NO: 525 - DNA, 3093 bp, synthetic construct
  source 1..3093: mol_type = other DNA
atgttgtctcctagaactctgaatgcttgggtaaaggtgatcgaagaaaaggcattctcacccgaggttatccctatgtttactgcgttgagcgaaggcgcaacaccccatgatctgaacacaatgctgaatacaatcggcggacatcaagctgctatgcaaatgcttaaggacaccatcaatgaggaggcagccgagtgggatcgcgttcatccagtccacgctgggcccgttgcgcctggtcagatgagggacccacgaggatccgacatcgcagggagcaccagtacactccaagagcagattgcatggatgacgaacaatccaccaatacctgtcggtgacatttacaaaaggtggattattatgggcttgaacaaaattgtacggatgtatagcccggtgagcatactggacattaaacagggtccaaaagaaccctttcgagattacgttgacagattttataggacactgagggcggaacaagcgtctcaagacgttaaaaactggatgacagaaacgctgcttgtacagaattccaacccagactgcaaaactatactgaaagcgctcggtcccggtgcgacacttgaggaaatgatgagcgcatgccagggcgtcggagggccgtctcacaaggcccgcgtgttggcggaagctatgtgccaggtggctaaggagatagtagcatgttgtgataaatgtcagcttaaaggtgaagctatacatgggcaggtggattgtagcccgggtgtatggcagctggactgtactcatctggaaggaaaggtaatacttgtcgcagttcatgtcgcgagcggatacattgaagctgaaatcattcctacggagacgggccaagagacagcttacttcatacttaaacttgctgggcgatggccggtgacaactatggcggcgcgcgcttcaattttgagtggtggaaagttggacaagtgggagaagattagactcagacccggagggagaaagaagtataaactgaaacatctggtttgggcttcacgcgaacttgaacggtttgctctcaaccccgggctgctcgaaactgctgctgctgtaaaggccgcttgttggtgggcgggggtaaagcaggaattcggaattccatataacactcaaagccagggagtagtggaatccatgaataatgaacttaagaagataattggacagattcgcgatcaggctgaacatctcaagacggccgtacaaatggcagtattgattcataactttaaacggaagggcggcataggagagtattctgcgggagaacgcataatagatataattgcgactcaggggttctttccggattggcagaactatacgccggggccaggcattaggttccccctcacgtttggatggtgtttcaagttggtaccgttgctcattaaaaaagaaaaaatctacctggcctgggtcccggcgcacaagggtataggggggaacgagcaaattgataagctcgtgtcaagggcgaagcgcgcgccagtcaaacagaccttgaatttcgacctccttaagctcgctggagacgtcgaatccaaccctggcccgatgctgccacaaatcacattgtggcaacgacccattgtaacaataaaaatcgggggccagatcaaagaagcgctgcttgacaccggcgccgacgatacagtcctcgaggatatgaatttgccaggcaaatggaagccgaagatgattggcggcattggcggctttattaaggttaaacagtatgatcagtggggattgaccactcccgataagaagcatcagaaagatccgccttttctgtggatggggtacgaactgcaccctgatcgatggacggtccagccgatagagctgccggaaaaagaatcatggaccgtgaatgatattcaaaaactgatcggaaaactcaattgggcgtcccagatatatgctggcatcaaagttgcagcacaagaagaggaagaggtaggtttcccggttcggccgcaagttcccttgcgaccgatgacatacaagggcgcattggacctttctcacttcctcaaggaaaagggcggtttggagggcatcactaaacttcagaatttcagagtctactatagagataacagggacccattgtggaagggccccgctcgacttctctggaaaggggagggagcggttgtaattcaagacaacagtgaaattaaggtcgtcccacgacggaaggttaaaataattcgcgactatggcaagcgaatggcgggagacgactgtgtagcaggacgacaagacgaggacccaaagtttagattgccgatccagaaagagacatgggatacgtggtggacggactattggcaggccacctggataccagaatgggagtttacaaacactcctccactcgtgaaattgtggtatcaacttgagaccgagcccatagctggtgtagagacgttttacgttgacggtgctagcaacagggaaacaaaggccgctggaaccgtgctcatcggtcctactcctgtgaacataattggacgaaatttgttgacccagctgggatgcaccctcaatttccccattagcccaatagataccgtaccagtcaagcttaagcctggtatggacggtccgcgagttaagcaatggccacttactgaggagaaaatcaaggcactcatcgagatctgcaccgaaatggagaaggagggcaaaataagcaggattggtcccgagaatccatataatacgccgatcttcgcgataaaaaagaaggacggcaccaaatggcgaaaactggttgacttccgggagcttaacaaaaaaactcaggatttttgggaagttcaactcgggatcccacacccgtctggtcttaaaaaaaaaaaaagcgtaacagtccttgacatcggcgatgcctactttagcgtgcctctcgataaggagttcagaaaatacacggctttcactgtaccaagcacaaacaatgagactcctggggtcagataccagtacaatgtccttcccatggggtggaaaggaagccccgcaatattccagtgctcaatgacgtga

SEQ ID NO: 526 - DNA, 3090 bp, synthetic construct
  source 1..3090: mol_type = other DNA
atgctttcaccgagaaccttgaacgcgtgggttaaagtaatcgaagagaaggcatttagcccggaggtaatccccatgtttaccgcactttccgaaggtgctacaccccacgacctgaatacaatgttgaatactattggcggccaccaagctgccatgcagatgcttaaggacactattaacgaggaagccgccgaatgggatcgcgttcaccctgttcacgcgggtccagtcgcaccaggacaaatgcgagaccctcgggggtctgacatcgcaggatcaactagtacattgcaggaacagattgcttggatgaccaataaccctccgatcccggtaggtgacatttataaacggtggatcatcatgggattgaacaaaatagtccgaatgtacagtccagtgagtattcttgacattaaacagggaccaaaggagccgttccgggactacgttgaccggttttacaggaccctgagagccgaacaggcttcccaggatgtgaaaaactggatgacagagaccctgcttgttcagaattcaaatcctgactgtaaaacgattctgaaggcactcggtcccggcgccacgctggaggaaatgatgtcagcttgtcagggagtaggaggaccttctcataaggcacgcgtccttgcagaggccatgtgccaagtcgcgaaagagatcgtggcctgttgtgacaagtgccaacttaagggagaagccatccatgggcaggtcgattgttctccgggcgtttggcaattggactgcacgcacttggagggtaaggtgattcttgttgccgttcatgtcgcaagcggttacattgaagcggagattatcccaactgagaccggccaagagactgcatatttcatccttaaactggcgggaagatggccggtcacgactatggctgcacgagcatccatactgtctgggggaaagctcgacaagtgggagaagattcgacttaggcctggaggaagaaaaaagtataagttgaaacatcttgtttgggcatcacgagaacttgaacggtttgctctgaatccgggattgcttgaaaccgccgcggctgtaaaggcagcatgttggtgggctggtgtcaaacaagagttcggtattccctataacacccaaagtcaaggcgtcgtagaatccatgaacaatgaacttaagaaaattatcggacaaatccgggaccaagcggaacacctcaaaactgctgtccagatggcagttctgattcataactttaaaagaaagggggggataggggagtattctgcaggtgaacgaattatagacataattgccacacaagggttttttccagattggcaaaattatacaccgggccctgggataagattcccgctcaccttcggctggtgcttcaaactggtacccttgctgatcaagaaagagaagatctatcttgcttgggtgccagcccataaagggatcgggggtaacgaacaaatcgataagctggtgtctagagctaaacgggctcccgtaaaacagaccttgaacttcgatctgcttaaattggcaggggacgtggaaagcaaccccgggccaatgctgccccagataacactttggcaacgccccatcgtgacaatcaagatcggtggccaaattaaggaagcactcttggacacgggagcagacgacactgtgctggaggatatgaacctgccgggcaagtggaaaccaaagatgatcgggggcattggcgggttcataaaggttaaacagtacgaccaatgggggttgacaacgcctgataagaagcatcaaaaagatcccccatttttgtggatgggttatgaacttcacccggacaggtggaccgttcagccgatagagctcccagaaaaggagtcttggacagttaatgacatacagaaacttattggcaaacttaactgggcttcacagatttatgccggcatcaaagtcgccgcccaggaagaagaagaggtaggttttcccgtacgacctcaggttcctcttcggcctatgacctataagggtgcgcttgatctttctcacttccttaaagaaaaaggaggtctggaaggtatcacgaaacttcagaattttcgggtgtattaccgggacaacagagacccgctttggaaggggccggctaggcttctgtggaaaggcgagggagcggtagttatccaggataactctgagataaaggtagtaccccgacggaaggtaaagatcatcagagactacggcaagaggatggctggagacgactgtgtggccgggcgacaggatgaagatcctaaattcaggctgccaatccaaaaagagacgtgggacacatggtggaccgattattggcaggctacgtggatccccgaatgggagtttaccaatactccgccactcgtgaagctttggtaccaattggagacagagcctatagccggcgttgagaccttctacgtggatggggccagcaacagagaaaccaaagcggccggaacggtcctgatcggtcccacacctgttaacatcatagggcgcaatctgcttacgcaattggggtgcacattgaattttccaatatcccctattgataccgtgccggttaaattgaagccgggtatggacgggcctcgggtcaagcagtggcccctgaccgaagaaaagatcaaagccctgattgagatctgcacggaaatggaaaaagagggcaagattagtcgcatcggcccggagaacccatacaatactcctatttttgcaattaaaaaaaaggacggaacaaagtggaggaaacttgtagatttcagagagcttaataagaaaactcaggacttctgggaggtccaactcggtattccgcatccctccggacttaagaagaaaaagtcagtaaccgtcttggatataggggacgcttatttttcagtgcccctcgataaagaatttcgcaaatacacggcgtttactgtgccatctactaataacgaaacgccaggcgtgagatatcaatacaacgtccttcctatgggctggaagggttcacccgcaatttttcagtgctccatgacc

SEQ ID NO: 527 - protein, 1031 aa, Human immunodeficiency virus 1
MISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQPPVVAKEIVASCDKCQLKGEAMHGQVDCSPGIWQLDCTHLEGKIILVAVHVASGYIEAEVIPAETGQETAYFLLKLAGRWPVKTMAARASVLSGGELDRWEKIRLRPGGKKKYRLKHIVWASRELERFAVNPGLLETAATVKAACWWAGIKQEFGIPYNPQSQGVVESMNKELKKIIGQVRDQAEHLKTAVQMAVFIHNFKRKGGIGGYSAGERIVDIIATQGYFPDWQNYTPGPGTRYPLTFGWCFKLVPVKEKVYLAWVPAHKGIGGNEQVDKLVSRAKRAPVKQTLNFDLLKLAGDVESNPGPFPQITLWQRPLVTIKIGGQLKEALLDTGADDTVLEEMNLPGRWKPKMIGGIGGFIKVRQYDQWGFTTPDKKHQKEPPFLWMGYELHPDKWTVQPIVLPEKDSWTVNDIQKLVGKLNWASQIYPGIKVAAQEEEEVGFPVKPQVPLRPMTFKGALDLSHFLREKGGLEGLIITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 528 - protein, 1030 aa, Human immunodeficiency virus 1
MLSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQVAKEIVACCDKCQLKGEAIHGQVDCSPGVWQLDCTHLEGKVILVAVHVASGYIEAEIIPTETGQETAYFILKLAGRWPVTTMAARASILSGGKLDKWEKIRLRPGGRKKYKLKHLVWASRELERFALNPGLLETAAAVKAACWWAGVKQEFGIPYNTQSQGVVESMNNELKKIIGQIRDQAEHLKTAVQMAVLIHNFKRKGGIGEYSAGERIIDIIATQGFFPDWQNYTPGPGIRFPLTFGWCFKLVPLLIKKEKIYLAWVPAHKGIGGNEQIDKLVSRAKRAPVKQTLNFDLLKLAGDVESNPGPMLPQITLWQRPIVTIKIGGQIKEALLDTGADDTVLEDMNLPGKWKPKMIGGIGGFIKVKQYDQWGLTTPDKKHQKDPPFLWMGYELHPDRWTVQPIELPEKESWTVNDIQKLIGKLNWASQIYAGIKVAAQEEEEVGFPVRPQVPLRPMTYKGALDLSHFLKEKGGLEGITKLQNFRVYYRDNRDPLWKGPARLLWKGEGAVVIQDNSEIKVVPRRKVKIIRDYGKRMAGDDCVAGRQDEDPKFRLPIQKETWDTWWTDYWQATWIPEWEFTNTPPLVKLWYQLETEPIAGVETFYVDGASNRETKAAGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 529 - DNA, 24 bp, unidentified
  source 1..24: mol_type = other DNA
ctctctacggctaacctgaatgga

SEQ ID NO: 530 - DNA, 602 bp, unidentified
  source 1..602: mol_type = other DNA
gacattgattattgactagttgttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcag

SEQ ID NO: 531 - protein, 438 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLASCSRSTSESTAALGCLVKDYFPEPVTVSWNSGALTSGVHTFPAVLQSSGLYSLSSVVTVPSSSLGTQTYVCNVVHEPSNTKVDKRVEFTPPCPPCPAPELLGGPSVFLFPPKPKDTLMISRTPEVTCVVVDVSQEDPEVQFNWYVDGAEVHHAQTKPRERQFNSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYFLYSKLTVDKSRWQPGNIFTCSVMHEALHNHYTQKSLSVSPGK

SEQ ID NO: 532 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRAVAAPSVFIFPPSEDQVKSGTVSVVCLLNNFYPREASVKWKVDGVLKTGNSQESVTEQDSKDNTYSLSSTLTLSSTDYQSHNVYACEVTHQGLSSPVTKSFNRGEC

SEQ ID NO: 533 - protein, 381 aa, synthetic construct
TQDCSFQHSPISSDFAVKIRELSDYLLQDYPVTVPSNLQDEELCGALWRLVLAQRWMERLKTVAGSKMQGLLERVNTEIHFVTKCAFQHPPSCLRFVQTNISRLLQETSEQLVALKPWITRQNFSRCLELQCQPDSSTLPPPRSPGALEATAGGSKPPTCPPCPAPEAAGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPASIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSVSPGK

SEQ ID NO: 534 - DNA, 31486 bp, synthetic construct
  source 1..31486: mol_type = other DNA
catcatcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagtagggataacagggtaatgacattgattattgactagttgttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggaacgccatccacgctgttttgacctccatagaagacagcgatcgcgccaccatgggagccaggaacagtgtgctctcaggcaagaaggcagatgagctggagaagatcaggctgagacccaatggcaagaagaagtacatgctgaagcatgtggtctgggcagccaatgagctggacaggtttggcctggcagagtccctgctggacaacaaggagggctgccagaagatcctgtcagtgctggcccccctggtgcccactggctcagagaacctgaagagtctctacaacacagtgtgtgtgatttggtgcatccatgcagaggagaaggtgaagcacactgaggaggccaagcagattgtgcagaggcacctggtggtggagactggcacagctgacaagatgccagccacctccaggcccacagcacccccctctggcagggggggcaactacccagtccagcaagtggggggcaactatgtgcacctgcccctgagccccagaaccctgaatgcctgggtcaagctggtggaggagaagaagtttggagcagaggtggtgcctggcttccaggccctgtcagagggatgcactccctatgacatcaaccagatgctgaactgtgtgggagagcaccaggcagcaatgcagatcatcagggagatcatcaatgaggaggctgctgactgggacctccagcacccccagcctggacccctccctgcaggccagctgagggagcccagagggagtgacatagcaggcaccacctccacagtggaggagcagatccagtggatgtacaggcagcagaaccccatccctgtgggcaacatctacaggaggtggatccagctgggcctccagaagtgtgtcaggatgtacaacccaaccaacatcctggatgtgaagcagggccccaaggagcccttccagtcttatgtggacaggttctacaagagcctgagagctgagcagacagaccctgctgtgaagaactggatgacccagacactgctgatccagaatgccaatcctgactgcaagctggtgctgaaggggctggggatgaatccaaccctggaggagatgctgacagcctgccagggcattgggggacctggacagaaggccaggctcatggcagaggctctcaaggaggccctcagaccagaccagctgccatttgctgctgtgcagcagaagggccagaggaggaccatcaagtgctggaactgtggcaaggagggccactctgccaggcagtgcagagcccccaggaggcagggctgctggggctgtggaaagacaggccatgtgatggccaagtgcccagagaggcaggcaggcttcctgggctttggcccctggggcaagaagccaagaaacttccccatggcccagatgccccagggcctgacccccacagcccccccagaggacccagctgtggacctgctgaagaactacatgaagatgggcaggaagcagagggagaacagggagagaccctacaaggaggtgactgaggacctgctgcacctgaactccctgtttggggaggaccagtgataggtttaaactcccatttaaatgtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataactataacggtcctaaggtagcgagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagtacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtggacgtttagagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatg

SEQ ID NO: 535 - DNA, 32068 bp, synthetic construct
  source 1..32068: mol_type = other DNA
catcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagtagggataacagggtaatgacattgattattgactagttgttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggaacgccatccacgctgttttgacctccatagaagacagcgatcgcgccaccatgggctgcctgggcaaccagctgctgatcgccctgctgctggtgtccgtgctggaaatctgctgcgtgcagtacgtgaccgtgttctacggcgtgcctgcctggaagaacgccaccatccccctgttctgcgccacccggaaccgggacacctggggcaccacacagtgcctgcccgacaacgacgactacagcgagctggccgtgaacatcaccgaggccttcgacgcctggaacaacaccgtgaccgagcaggccatcgaggacgtgtggaacctgttcgagacaagcatcaagccctgcgtgaagctgacacccctgtgtatcgccatgcggtgcaacaagaccgagacagacagatggggactgaccggcagggctgagacaaccaccaccgccaagagcaccacctctaccaccaccacaaccgtgacccccaaggtgatcaacgagggcgacagctgcatcaagaacaacagctgtgctggcctcgaacaggaacccatgatcggctgcaagttcaacatgacaggcctgaagcgggacaagaagatcgagtacaacgagacatggtactctcgggacctgatctgcgagcagcctgccaacggcagcgagagcaagtgttacatgcagcattgcaacaccagcgtgatccaggaaagctgcgacaagcactactgggacgccatccggttccggtactgcgctcctcctggctacgccctgctgaggtgcaacgacagcaactacagcggcttcgctcccaagtgcagcaaggtggtggtgtccagctgcacccggatgatggaaacccagaccagcacctggttcggcttcaacggcaccagagccgagaaccggacctacatctactggcacggcaacagcaaccggaccatcatcagcctgaacaagtactacaacctgaccatgaagtgcagaaggcctggcaataagaccgtgctgcccgtgaccatcatgtctggcctggtgttccacagccagcccatcaacgagaggcccaagcaggcctggtgccggttcggagggaactggtccgaggccatccaggaagtgaaagaaaccctggtgaagcaccctcggtacaccggcaccaacgacacccggaagatcaacctgaccgctcctgctggcggagatcccgaggtgaccttcatgtggaccaactgcagaggcgagttcctgtactgcaagatgaactggttcctgaactgggtggaggaccgggaccagaacagcaacaggtggaagcagcagaagaagcccgagcagcagaaacggaactacgtgccctgccacatccggcagatcatcaacacctggcacaaagtgggcaagaacgtgtacctgcctcccagagagggcgacctgacctgcaactccaccgtgaccagcctgatcgccgagatcgactggatcaacaacaacgaaaccaacatcaccatgtctgccgaggtggccgagctgtacaggctggaactgggcgactacaaactggtggagatcacccccatcggcctggctcccaccgacgtcagacggtacaccaccacaggagccagccggaacaagagaggcgtctttgtcctgggcttcctcggctttctggccacagcaggctctgcaatgggagcagccagcctgaccctgtctgcccagagccggacactgctggcaggcattgtgcagcagcagcaacagctcctggacgtggtgaagcggcagcatgaactcctgcgactgaccgtgtggggcaccaagaacctgcagacacgggtgaccgccatcgagaagtacctgaaggaccaggctcagctcaactcttggggctgcgccttccggcaggtctgccacaccaccgtgccctggcccaacgactccctggtgcccaactgggacaacatgacctggcaggaatgggagggcaaggtcgacttcctggaagccaatatcacccagctgctggaagaggcccagatccagcaggaaaagaacatgtacgagctgcagaagctgaactcctgggacatcttcggcaattggttcgacctgacctcctggattagatacatccagtaatgataggtttaaactcccatttaaatgtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataactataacggtcctaaggtagcgagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagtacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtggacgtttagagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatg

SEQ ID NO: 536 - DNA, 3045 bp, synthetic construct
  source 1..3045: mol_type = other DNA
atgcgcgtgtggccaatgggaaaagaagcaccacagttcccccatggcccagatgcaagtggagctgataccaactgcagccccagaggctctagctgcggcagcaccgaggaactgcacgaggacgggcagaaggccgagggcgagcagcgggaaaccctccagggaggagatagaggctttgctgcccctcagttcagcctgtggcggagacccgtggtgaccgcctacatcgaggaacagcccgtggaggtgctgctggccacaggcgctgacgacagcatcgtgaccggcatcgagctgggacccaactacacccccaagatcgtgggaggaatcggaggattcatcaacaccggcgaatacaaggacgtgaagatcaaggtgctcggcaaggtcatcaagggcaccatcatgacaggcgacacccccatcaacatctttggcagaaacctgctgacagccatgggcatgagcctgaacttccctatcgccaaggtggagcccatcaaggtgaccctgaagcctgggaaagaaggccctaagctgaggcagtggcctctgtccaaagagaagatcattgccctgcgggagatctgcgagaagatggaaaaggacggccagctggaagaggctcctcccaccaacccctacaacacccccaccttcgccatcaagaagaaagacaagaacaagtggcggatgctgatcgacttccgggagctgaataaggtgacccaggacttcaccgaggtgcagctgggcatccctcaccctgctggcctcgcaaagaggcggaggatcaccgtcctggacgtgggcgacgcctactttagcattcccctggacgaagagttccggcagtacaccgccttcaccctgcccagcgtgaacaacgccgagcctggcaagcggtacatctacaaggtgctgcctcagggctggaagggctctcctgccatcttccagtacaccatgcggaacgtgctggaaccctttcggaaggccaaccctgacgtgaccctgatccagtacatggaccacatcctgatcgccagcgaccggaccgacctggaacatgacagagtcgtgctccagctgaaagagctgctgaacggcatcggcttcagcacacccgaagagaagttccagaaggaccctccctttcagtggatgggctatgaactctggcccaccaagtggaagctgcagaagatcgagctgccccagcgggagacatggaccgtgaacgacatccagaaactggtcggcgtgctgaactgggctgcccagatctaccctggcatcaagaccaagcacctgtgtcggctgatcagaggcaagatgaccctgaccgaggaagtgcagtggaccgagatggccgaagccgagtacgaggaaaacaagatcatcctgagccaggaacaggaaggctgctactaccaggaaggcaagcccatcgaggccaccgtgatcaagagccaggacaaccagtggagctacaagatccaccaggaagataaagtcctgaaagtgggcaagttcgccaaagtgaagaacacccacaccaacggcgtgcggctgctggcccacgtggtgcagaagattggcaaggaagccctggtgatctggggagaggtgcccaagttccacctgcccgtggagcgcgagatctgggagcagtggtggaccgactactggcaggtgacctggattcccgactgggatttcgtgagcacaccccctctggtccggctcgtgttcaacctggtgaaagagcctatccagggagccgagacattctacgtggacggcagctgcaaccggcagagcagggagggcaaggctggctatgtgaccgacaggggcagggacaaggccaagctgctggaacagaccaccaaccagcaggccgagctggaagccttctacctggccctggccgactctggccctaaggccaacatcatcgtggacagccagtacgtgatgggcatcgtggctggacagcccaccgagagcgagtctcggctggtgaaccagatcatcgaggaaatgatcaagaaagaggccatctacgtcgcctgggtgccagcccacaagggcattggaggaaaccaggaagtggaccacctggtctcccagggcatccggcaggtgctgtttctggaaaaaatcgagcctgcccaggaagagcacgagaagtaccacagcaacgtgaaagaactggtgtttaagttcggcatccccagactggtggccaaacagattgtggacacctgcgaccggtgccaccagaagggcgaggccatccacggccaggtcaacgccgagctgggcacctggcagatggcctgcacccacctggaaggcaagatcatcattgtggccgtgcacgtggccagcggcttcatcgaggccgaggtgatcccccaggaaacaggacgccagacagccctgttcctgctgaagctggctggcaggtggcccatcacccacctgcacaccgacaatggcgctaacttcaccagccaggaagtgaagatggtcgcctggtgggctggcatcgaacagaccttcggcgtgccctacaacccccagagccagggcgtggtggaggccatgaaccaccacctgaaaacccagatcgaccggatcagagagcaggccaacagcgtcgagacaatcgtgctgatggccgtgcactgcatgaacttcaagcggagaggagggatcggcgacatgacccctgccgagagactggtgaacatgatcaccaccgagcaggaaatccagttccagcagagcaagaacagcaagttcaagaacttccgggtgtactaccgggagggcagggaccagctgtggagaggccctggcgagctgctgtggaagggcgaaggcgctgtgatcctgaaagtcgggaccgagatcaaggtggtgcccaggcggaaggccaagattatcaaggactatggcggagggaaagagctggacagcggctctcacctggaagatacaggcgaggcacgggaggtggcctaa

SEQ ID NO: 537 - DNA, 10039 bp, synthetic construct
  source 1..10039: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactctagaatagtctttaattaagccaccatgggctgcctgggcaaccagctgctgatcgccctgctgctggtgtccgtgctggaaatctgctgcgtgcagtacgtgaccgtgttctacggcgtgcctgcctggaagaacgccaccatccccctgttctgcgccacccggaaccgggacacctggggcaccacacagtgcctgcccgacaacgacgactacagcgagctggccgtgaacatcaccgaggccttcgacgcctggaacaacaccgtgaccgagcaggccatcgaggacgtgtggaacctgttcgagacaagcatcaagccctgcgtgaagctgacacccctgtgtatcgccatgcggtgcaacaagaccgagacagacagatggggactgaccggcagggctgagacaaccaccaccgccaagagcaccacctctaccaccaccacaaccgtgacccccaaggtgatcaacgagggcgacagctgcatcaagaacaacagctgtgctggcctcgaacaggaacccatgatcggctgcaagttcaacatgacaggcctgaagcgggacaagaagatcgagtacaacgagacatggtactctcgggacctgatctgcgagcagcctgccaacggcagcgagagcaagtgttacatgcagcattgcaacaccagcgtgatccaggaaagctgcgacaagcactactgggacgccatccggttccggtactgcgctcctcctggctacgccctgctgaggtgcaacgacagcaactacagcggcttcgctcccaagtgcagcaaggtggtggtgtccagctgcacccggatgatggaaacccagaccagcacctggttcggcttcaacggcaccagagccgagaaccggacctacatctactggcacggcaacagcaaccggaccatcatcagcctgaacaagtactacaacctgaccatgaagtgcagaaggcctggcaataagaccgtgctgcccgtgaccatcatgtctggcctggtgttccacagccagcccatcaacgagaggcccaagcaggcctggtgccggttcggagggaactggtccgaggccatccaggaagtgaaagaaaccctggtgaagcaccctcggtacaccggcaccaacgacacccggaagatcaacctgaccgctcctgctggcggagatcccgaggtgaccttcatgtggaccaactgcagaggcgagttcctgtactgcaagatgaactggttcctgaactgggtggaggaccgggaccagaacagcaacaggtggaagcagcagaagaagcccgagcagcagaaacggaactacgtgccctgccacatccggcagatcatcaacacctggcacaaagtgggcaagaacgtgtacctgcctcccagagagggcgacctgacctgcaactccaccgtgaccagcctgatcgccgagatcgactggatcaacaacaacgaaaccaacatcaccatgtctgccgaggtggccgagctgtacaggctggaactgggcgactacaaactggtggagatcacccccatcggcctggctcccaccgacgtcagacggtacaccaccacaggagccagccggaacaagagaggcgtctttgtcctgggcttcctcggctttctggccacagcaggctctgcaatgggagcagccagcctgaccctgtctgcccagagccggacactgctggcaggcattgtgcagcagcagcaacagctcctggacgtggtgaagcggcagcatgaactcctgcgactgaccgtgtggggcaccaagaacctgcagacacgggtgaccgccatcgagaagtacctgaaggaccaggctcagctcaactcttggggctgcgccttccggcaggtctgccacaccaccgtgccctggcccaacgactccctggtgcccaactgggacaacatgacctggcaggaatgggagggcaaggtcgacttcctggaagccaatatcacccagctgctggaagaggcccagatccagcaggaaaagaacatgtacgagctgcagaagctgaactcctgggacatcttcggcaattggttcgacctgacctcctggattagatacatccagtaatgattcgaacggccgtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 538 - DNA, 1524 bp, synthetic construct
  source 1..1524: mol_type = other DNA
atgggagccaggaacagtgtgctctcaggcaagaaggcagatgagctggagaagatcaggctgagacccaatggcaagaagaagtacatgctgaagcatgtggtctgggcagccaatgagctggacaggtttggcctggcagagtccctgctggacaacaaggagggctgccagaagatcctgtcagtgctggcccccctggtgcccactggctcagagaacctgaagagtctctacaacacagtgtgtgtgatttggtgcatccatgcagaggagaaggtgaagcacactgaggaggccaagcagattgtgcagaggcacctggtggtggagactggcacagctgacaagatgccagccacctccaggcccacagcacccccctctggcagggggggcaactacccagtccagcaagtggggggcaactatgtgcacctgcccctgagccccagaaccctgaatgcctgggtcaagctggtggaggagaagaagtttggagcagaggtggtgcctggcttccaggccctgtcagagggatgcactccctatgacatcaaccagatgctgaactgtgtgggagagcaccaggcagcaatgcagatcatcagggagatcatcaatgaggaggctgctgactgggacctccagcacccccagcctggacccctccctgcaggccagctgagggagcccagagggagtgacatagcaggcaccacctccacagtggaggagcagatccagtggatgtacaggcagcagaaccccatccctgtgggcaacatctacaggaggtggatccagctgggcctccagaagtgtgtcaggatgtacaacccaaccaacatcctggatgtgaagcagggccccaaggagcccttccagtcttatgtggacaggttctacaagagcctgagagctgagcagacagaccctgctgtgaagaactggatgacccagacactgctgatccagaatgccaatcctgactgcaagctggtgctgaaggggctggggatgaatccaaccctggaggagatgctgacagcctgccagggcattgggggacctggacagaaggccaggctcatggcagaggctctcaaggaggccctcagaccagaccagctgccatttgctgctgtgcagcagaagggccagaggaggaccatcaagtgctggaactgtggcaaggagggccactctgccaggcagtgcagagcccccaggaggcagggctgctggggctgtggaaagacaggccatgtgatggccaagtgcccagagaggcaggcaggcttcctgggctttggcccctggggcaagaagccaagaaacttccccatggcccagatgccccagggcctgacccccacagcccccccagaggacccagctgtggacctgctgaagaactacatgaagatgggcaggaagcagagggagaacagggagagaccctacaaggaggtgactgaggacctgctgcacctgaactccctgtttggggaggaccagtga